(19) United States
(12) Patent Application Publication
(10) Pub. No.: US 2026/0034175 A1
(43) Pub. Date: Feb. 5, 2026

(54) COMPOSITIONS AND METHODS FOR HEMATOPOIETIC STEM CELL TRANSPLANTS

(71) Applicant: Orca Biosystems, Inc., Menlo Park, CA (US)

(72) Inventors: Nathaniel Fernhoff, Menlo Park, CA (US); Scott McClellan, Menlo Park, CA (US); Amy Putnam, Menlo Park, CA (US)

(21) Appl. No.: 19/197,902

(22) Filed: May 2, 2025

Related U.S. Application Data
(63) Continuation of application No. PCT/US2023/078721, filed on Nov. 3, 2023.
(60) Provisional application No. 63/422,375, filed on Nov. 3, 2022.
(60) Provisional application No. 63/520,234, filed on Aug. 17, 2023.

(51) Int. Cl. A61K 35/17; A61K 31/436; A61K 31/7076; A61K 35/28; A61P 35/02

(52) U.S. Cl. CPC A61K 35/17; A61K 31/436; A61K 31/7076; A61K 35/28; A61P 35/02

(57) ABSTRACT

The present disclosure provides distinct therapeutic populations of cells that form a pharmaceutical composition useful in hematopoietic stem/progenitor cell transplant. For example, the present disclosure provides a therapeutic population of cells, comprising an enriched population of hematopoietic stem/progenitor cells, memory T cells, regulatory T cells, and wherein the population of cells is depleted of naïve conventional αβ-T cells. The present disclosure further provides methods of treatment using the therapeutic population of cells. In other embodiments, the present disclosure provides methods of producing a therapeutic population of cells.

### CROSS-REFERENCE TO RELATED APPLICATIONS

This application is a continuation of International Application No. PCT/US2023/078721 filed Nov. 3, 2023, which claims the benefit of priority of U.S. Provisional Application Nos. 63/422,375 filed Nov. 3, 2022, and 63/520,234 filed Aug. 17, 2023, each of which is hereby incorporated by reference in their entirety for all purposes.

### BACKGROUND

Myeloablative (MA) allogeneic hematopoietic cell transplantation (alloHCT) is the standard treatment for a variety of hematological malignancies. However, MA-alloHCT is curative in only some patients and better strategies for alloHCT remain critically needed to improve patient outcomes.

### SUMMARY

An aspect of the present disclosure is a pharmaceutical composition comprising one or more unit doses of a cellular graft, wherein each unit dose of the cellular graft comprises populations of therapeutic cells for each kilogram (kg) of body weight of a subject receiving the cellular graft. The populations of therapeutic cells of each unit dose comprise: more than 3×10⁵ hematopoietic stem/progenitor cells (HSPC) per kilogram of body weight of the subject receiving the cellular graft, more than 3×10⁵ memory T cells (Tmem) per kilogram of body weight of the subject receiving the cellular graft, more than 5×10⁵ regulatory T cells (Treg) per kilogram of body weight of the subject receiving the cellular graft, and less than 3×10⁵ naïve conventional αβ-T cells per kilogram of body weight of the subject receiving the cellular graft. In this aspect, the subject receiving the cellular graft has received a myeloablative conditioning regimen comprising thiotepa and/or the subject receiving the cellular graft is receiving or will receive a GVHD prophylactic regimen comprising tacrolimus.

In embodiments, the unit dose further comprises 0.5×10¹ to 2000×10¹ invariant natural killer T (iNKT) cells per kilogram of body weight of the subject receiving the cellular graft.

In various embodiments, the HSPC are CD34+, the Tmem are CD3⁺CD45RA-CD45RO+, the Treg are CD4⁺CD25⁺CD127−/lo, CD45RA+, or a combination thereof, and the naïve conventional αβ-T cells are CD3⁺CD45RA+CD25−TCR Va24Ja18−.

In some embodiments, the iNKT are CD3+Vα24Jα18+.

In embodiments, the populations of therapeutic cells of each unit dose comprise: 1.0×10⁶ to 50×10⁶ HSPC per kilogram of body weight of the subject receiving the cellular graft, 0.3×10⁶ to 1000×10⁶ Tmem per kilogram of body weight of the subject receiving the cellular graft, 0.5×10⁶ to 1000×10⁶ Treg per kilogram of body weight of the subject receiving the cellular graft, and less than 3×10⁵ naïve conventional αβ-T cells per kilogram of body weight of the subject receiving the cellular graft.

In various embodiments, the unit dose further comprises iNKT cells, wherein the populations of therapeutic cells of each unit dose comprise: 1.0×10⁶ to 50×10⁶ HSPC per kilogram of body weight of the subject receiving the cellular graft, 0.3×10⁶ to 1000×10⁶ Tmem per kilogram of body weight of the subject receiving the cellular graft, 0.5×10⁶ to 1000×10⁶ Treg per kilogram of body weight of the subject receiving the cellular graft, 0.5×10³ to 2000×10³ iNKT per kilogram of body weight of the subject receiving the cellular graft, and less than 3×10⁵ naïve conventional αβ-T cells per kilogram of body weight of the subject receiving the cellular graft.

In some embodiments, the populations of therapeutic cells of each unit dose comprises 0.2×10⁶ to 500×10⁶ naive Treg cells per kilogram of body weight of the subject receiving the cellular graft.

In embodiments, the populations of therapeutic cells of each unit dose comprises 0.2×10⁶ to 500×10⁶ naive Treg cells per kilogram of body weight of the subject receiving the cellular graft.

In various embodiments, the populations of therapeutic cells of each unit dose comprises 1.0×10⁶ to 50×10⁶ HSPC per kilogram of body weight of the subject receiving the cellular graft.

In some embodiments, the populations of therapeutic cells of each unit dose comprises 1.0×10⁶ to 250×10⁶ Tmem per kilogram of body weight of the subject receiving the cellular graft.

In embodiments, the populations of therapeutic cells of each unit dose comprises less than 1×10⁵ naïve conventional αβ-T cells per kilogram of body weight of the subject receiving the cellular graft.

In various embodiments, the populations of therapeutic cells of each unit dose comprises 1×10⁶ to 2.5×10⁶ Treg cells per kilogram of body weight of the subject receiving the cellular graft.

In some embodiments, the unit dose further comprises 1×10⁴ to 200×10⁴ iNKT per kilogram of body weight of the subject receiving the cellular graft.

In embodiments, the pharmaceutical composition is suitable for administration to the subject receiving the cellular graft.

In various embodiments, the pharmaceutical composition further comprises an excipient. In some cases, the excipient is Normosol-R. In some cases, the excipient is human serum.

In some embodiments, the HSPC are haploidentical to the subject receiving the cellular graft.

In embodiments, the Treg, Tmem, or a combination thereof are HLA matched with the subject receiving the cellular graft.

In various embodiments, at least a portion of the populations of therapeutic cells are autologous to the subject receiving the cellular graft.

In some embodiments, the subject receiving the pharmaceutical composition or the cellular graft has received a myeloablative conditioning regimen comprising thiotepa.

In embodiments, the subject receiving the pharmaceutical composition or the cellular graft is receiving a GVHD prophylactic regimen comprising tacrolimus.

In various embodiments, the subject receiving the pharmaceutical composition or the cellular graft will receive a GVHD prophylactic regimen comprising tacrolimus.

Another aspect of the present disclosure is a pharmaceutical composition comprising a population of therapeutic cells that is enriched for hematopoietic stem/progenitor cells (HSPC), memory T cells (Tmem), and regulatory T cells (Treg), and wherein the population of cells is depleted of naïve conventional αβ-T cells, wherein the population of therapeutic cells comprises a ratio of naïve conventional αβ-T cells to HSPC that is less than 1:400, a ratio of naïve conventional αβ-T cells to Tmem less than 1:800, and a ratio of naïve conventional αβ-T cells to Treg less than 1:5. In this aspect, a subject receiving the pharmaceutical composition has received a myeloablative conditioning regimen comprising thiotepa and/or the subject receiving the cellular graft is receiving or will receive a GVHD prophylactic regimen comprising tacrolimus.

In some embodiments, the population of therapeutic cells further comprises invariant Natural Killer T cells (iNKT) and the population of therapeutic cells comprises a ratio of naïve conventional αβ-T cells to iNKT less than 100:1.

In embodiments, the ratio of naïve conventional αβ-T cells to Tmem is less than 1:3.

In various embodiments, the population of therapeutic cells comprises a ratio of naïve conventional αβ-T cells to naïve Treg less than 1:2.

In some embodiments, the subject receiving the pharmaceutical composition or the cellular graft has received a myeloablative conditioning regimen comprising thiotepa.

In embodiments, the subject receiving the pharmaceutical composition or the cellular graft is receiving a GVHD prophylactic regimen comprising tacrolimus.

In various embodiments, the subject receiving the pharmaceutical composition or the cellular graft will receive a GVHD prophylactic regimen comprising tacrolimus.

It shall be understood that different aspects and/or embodiments of the present disclosure can be appreciated individually, collectively, or in combination with each other. Any description herein concerning a specific composition and/or method apply to and may be used for any other specific composition and/or method as disclosed herein. Additionally, any composition disclosed herein is applicable to any herein-disclosed method. In other words, any aspect or embodiment described herein can be combined with any other aspect or embodiment as disclosed herein.

Certain aspects of the present disclosure relate to a cellular therapy product comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the cellular therapy product has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH.

In some embodiments, the dose of HSPC comprises a dose of approximately 1.0×10⁵ or more HSPC per kilogram of body weight of the human subject receiving the product, approximately 2.0×10⁵ or more HSPC per kilogram of body weight of the human subject receiving the product, approximately 3.0×10⁵ or more HSPC per kilogram of body weight of the human subject receiving the product, approximately 4.0×10⁵ or more HSPC per kilogram of body weight of the human subject receiving the product, approximately 5.0×10⁵ or more HSPC per kilogram of body weight of the human subject receiving the product, approximately 6.0×10⁵ or more HSPC per kilogram of body weight of the human subject receiving the product, approximately 7.0×10⁵ or more HSPC per kilogram of body weight of the human subject receiving the product, approximately 8.0×10⁵ or more HSPC per kilogram of body weight of the human subject receiving the product, approximately 9.0×10⁵ or more HSPC per kilogram of body weight of the human subject receiving the product, approximately 1.0×10⁶ or more HSPC per kilogram of body weight of the human subject receiving the product, approximately 1.5×10⁶ or more HSPC per kilogram of body weight of the human subject receiving the product, approximately 2.0×10⁶ or more HSPC per kilogram of body weight of the human subject receiving the product, approximately 2.5×10⁶ or more HSPC per kilogram of body weight of the human subject receiving the product, approximately 3.0×10⁶ or more HSPC per kilogram of body weight of the human subject receiving the product, approximately 3.5×10⁶ or more HSPC per kilogram of body weight of the human subject receiving the product, approximately 4.0×10⁶ or more HSPC per kilogram of body weight of the human subject receiving the product, approximately 4.5×10⁶ or more HSPC per kilogram of body weight of the human subject receiving the product, approximately 5.0×10⁶ or more HSPC per kilogram of body weight of the human subject receiving the product, approximately 5.5×10⁶ or more HSPC per kilogram of body weight of the human subject receiving the product, approximately 6.0×10⁶ or more HSPC per kilogram of body weight of the human subject receiving the product, approximately 6.5×10⁶ or more HSPC per kilogram of body weight of the human subject receiving the product, approximately 7.0×10⁶ or more HSPC per kilogram of body weight of the human subject receiving the product, approximately 7.5×10⁶ or more HSPC per kilogram of body weight of the human subject receiving the product, approximately 8.0×10⁶ or more HSPC per kilogram of body weight of the human subject receiving the product, approximately 8.5×10⁶ or more HSPC per kilogram of body weight of the human subject receiving the product, approximately 9.0×10⁶ or more HSPC per kilogram of body weight of the human subject receiving the product, approximately 9.5×10⁶ or more HSPC per kilogram of body weight of the human subject receiving the product, approximately 1.0×10⁷ or more HSPC per kilogram of body weight of the human subject receiving the product, approximately 1.5×10⁷ or more HSPC per kilogram of body weight of the human subject receiving the product, approximately 2.0×10⁷ or more HSPC per kilogram of body weight of the human subject receiving the product, approximately 2.5×10⁷ or more HSPC per kilogram of body weight of the human subject receiving the product, approximately 3.0×10⁷ or more HSPC per kilogram of body weight of the human subject receiving the product, approximately 3.5×10⁷ or more HSPC per kilogram of body weight of the human subject receiving the product, approximately 4.0×10⁷ or more HSPC per kilogram of body weight of the human subject receiving the product, approximately 4.5×10⁷ or more HSPC per kilogram of body weight of the human subject receiving the product, approximately 5.0×10⁷ or more HSPC per kilogram of body weight of the human subject receiving the product, approximately 5.5×10⁷ or more HSPC per kilogram of body weight of the human subject receiving the product, approximately 6.0×10⁷ or more HSPC per kilogram of body weight of the human subject receiving the product, approximately 6.5×10⁷ or more HSPC per kilogram of body weight of the human subject receiving the product, approximately 7.0×10⁷ or more HSPC per kilogram of body weight of the human subject receiving the product, approximately 7.5×10⁷ or more HSPC per kilogram of body weight of the human subject receiving the product, approximately 8.0×10⁷ or more HSPC per kilogram of body weight of the human subject receiving the product, approximately 8.5×10⁷ or more HSPC per kilogram of body weight of the human subject receiving the product, approximately 9.0×10⁷ or more HSPC per kilogram of body weight of the human subject receiving the product, approximately 9.5×10⁷ or more HSPC per kilogram of body weight of the human subject receiving the product, or approximately 1.0×10⁸ or more HSPC per kilogram of body weight of the human subject receiving the product. In some embodiments that may be combined with any of the preceding embodiments, the dose of HSPC comprises a total dose of approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC. In some embodiments that may be combined with any of the preceding embodiments, the dose of Tmem comprises a dose of approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product. In some embodiments that may be combined with any of the preceding embodiments, the dose of Tmem comprises a total dose of approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem. In some embodiments that may be combined with any of the preceding embodiments, the dose of fresh Treg comprises a dose of approximately 1.0×10⁵ or more fresh Treg per kilogram of body weight of the human subject receiving the product, approximately 2.0×10⁵ or more fresh Treg per kilogram of body weight of the human subject receiving the product, approximately 3.0×10⁵ or more fresh Treg per kilogram of body weight of the human subject receiving the product, approximately 4.0×10⁵ or more fresh Treg per kilogram of body weight of the human subject receiving the product, approximately 5.0×10⁵ or more fresh Treg per kilogram of body weight of the human subject receiving the product, approximately 6.0×10⁵ or more fresh Treg per kilogram of body weight of the human subject receiving the product, approximately 7.0×10⁵ or more fresh Treg per kilogram of body weight of the human subject receiving the product, approximately 8.0×10⁵ or more fresh Treg per kilogram of body weight of the human subject receiving the product, approximately 9.0×10⁵ or more fresh Treg per kilogram of body weight of the human subject receiving the product, approximately 1.0×10⁶ or more fresh Treg per kilogram of body weight of the human subject receiving the product, approximately 1.5×10⁶ or more fresh Treg per kilogram of body weight of the human subject receiving the product, approximately 2.0×10⁶ or more fresh Treg per kilogram of body weight of the human subject receiving the product, approximately 2.5×10⁶ or more fresh Treg per kilogram of body weight of the human subject receiving the product, approximately 3.0×10⁶ or more fresh Treg per kilogram of body weight of the human subject receiving the product, approximately 3.5×10⁶ or more fresh Treg per kilogram of body weight of the human subject receiving the product, approximately 4.0×10⁶ or more fresh Treg per kilogram of body weight of the human subject receiving the product, approximately 4.5×10⁶ or more fresh Treg per kilogram of body weight of the human subject receiving the product, approximately 5.0×10⁶ or more fresh Treg per kilogram of body weight of the human subject receiving the product, approximately 5.5×10⁶ or more fresh Treg per kilogram of body weight of the human subject receiving the product, approximately 6.0×10⁶ or more fresh Treg per kilogram of body weight of the human subject receiving the product, approximately 6.5×10⁶ or more fresh Treg per kilogram of body weight of the human subject receiving the product, approximately 7.0×10⁶ or more fresh Treg per kilogram of body weight of the human subject receiving the product, approximately 7.5×10⁶ or more fresh Treg per kilogram of body weight of the human subject receiving the product, approximately 8.0×10⁶ or more fresh Treg per kilogram of body weight of the human subject receiving the product, approximately 8.5×10⁶ or more fresh Treg per kilogram of body weight of the human subject receiving the product, approximately 9.0×10⁶ or more fresh Treg per kilogram of body weight of the human subject receiving the product, approximately 9.5×10⁶ or more fresh Treg per kilogram of body weight of the human subject receiving the product, approximately 1.0×10⁷ or more fresh Treg per kilogram of body weight of the human subject receiving the product, approximately 1.5×10⁷ or more fresh Treg per kilogram of body weight of the human subject receiving the product, or approximately 2.0×10⁷ or more fresh Treg per kilogram of body weight of the human subject receiving the product. In some embodiments that may be combined with any of the preceding embodiments, the dose of fresh Treg comprises a total dose of approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg. In some embodiments that may be combined with any of the preceding embodiments, the cellular therapy product comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product. In some embodiments that may be combined with any of the preceding embodiments, the cellular therapy product comprises less than approximately 6.0×10⁷ total Tcon, less than approximately 5.5×10⁷ total Tcon, less than approximately 5.0×10⁷ total Tcon, less than approximately 4.5×10⁷ total Tcon, less than approximately 4.0×10⁷ total Tcon, less than approximately 3.5×10⁷ total Tcon, less than approximately 3.0×10⁷ total Tcon, less than approximately 2.5×10⁷ total Tcon, less than approximately 2.0×10⁷ total Tcon, less than approximately 1.5×10⁷ total Tcon, less than approximately 1.0×10⁷ total Tcon, less than approximately 9.5×10⁶ total Tcon, less than approximately 9.0×10⁶ total Tcon, less than approximately 8.5×10⁶ total Tcon, less than approximately 8.0×10⁶ total Tcon, less than approximately 7.5×10⁶ total Tcon, less than approximately 7.0×10⁶ total Tcon, less than approximately 6.5×10⁶ total Tcon, less than approximately 6.0×10⁶ total Tcon, less than approximately 5.5×10⁶ total Tcon, less than approximately 5.0×10⁶ total Tcon, less than approximately 4.5×10⁶ total Tcon, less than approximately 4.0×10⁶ total Tcon, less than approximately 3.5×10⁶ total Tcon, less than approximately 3.0×10⁶ total Tcon, less than approximately 2.5×10⁶ total Tcon, less than approximately 2.0×10⁶ total Tcon, less than approximately 1.5×10⁶ total Tcon, or less than approximately 1.0×10⁶ total Tcon. In some embodiments that may be combined with any of the preceding embodiments, the population of isolated CD45⁺ cells is formulated at a volume that ranges from approximately 5 mL to approximately 1 L. In some embodiments that may be combined with any of the preceding embodiments, the population of isolated CD45⁺ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L. In some embodiments that may be combined with any of the preceding embodiments, the neutral pH ranges from approximately 6.8 to approximately 7.6. In some embodiments that may be combined with any of the preceding embodiments, the excipient comprises a transport buffer. In some embodiments that may be combined with any of the preceding embodiments, the transport buffer comprises approximately 120 to approximately 160 mEq sodium. In some embodiments that may be combined with any of the preceding embodiments, the transport buffer comprises approximately 270 to approximately 320 mOsmol/L total. In some embodiments that may be combined with any of the preceding embodiments, the transport buffer is selected from: phosphate-buffered saline (PBS), human serum, PlasmaLyte, Normosol-R, and any combination thereof. In some embodiments that may be combined with any of the preceding embodiments, the transport buffer further comprises approximately 0.1% volume by volume to approximately 10% volume by volume of a human carrier protein. In some embodiments that may be combined with any of the preceding embodiments, the human carrier protein is selected from: human serum albumin (HSA), intravenous immune globulin (IVIG), AB serum, and any combination thereof. In some embodiments that may be combined with any of the preceding embodiments, the population of isolated CD45⁺ cells is formulated in a single dose transfer bag. In some embodiments that may be combined with any of the preceding embodiments, the single dose transfer bag is a polyvinyl chloride (PVC) transfer bag or an ethylene vinyl acetate (EVA) transfer bag. In some embodiments that may be combined with any of the preceding embodiments, the population of isolated CD45⁺ cells is enriched for HSPC, Tmem, and Treg. In some embodiments that may be combined with any of the preceding embodiments, the population of isolated CD45⁺ cells comprises a ratio of Tcon to HSPC that is less than 1:3, less than 1:5, less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, less than 1:50,000, less than 1:55,000, less than 1:60,000, less than 1:65,000, less than 1:70,000, less than 1:75,000, less than 1:80,000, less than 1:85,000, less than 1:90,000, less than 1:95,000, less than 1:100,000, less than 1:200,000, less than 1:300,000, less than 1:400,000, less than 1:500,000, less than 1:600,000, less than 1:700,000, less than 1:800,000, less than 1:900,000, or less than 1:1,000,000. In some embodiments that may be combined with any of the preceding embodiments, the population of isolated CD45⁺ cells comprises a ratio of Tcon to Tmem this is less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000. In some embodiments that may be combined with any of the preceding embodiments, the population of isolated CD45⁺ cells comprises a ratio of Tcon to Treg that is less than 1:1, less than 1:2, less than 1:3, less than 1:4, less than 1:5, less than 1:6, less than 1:7, less than 1:8, less than 1:9, less than 1:10, less than 1:11, less than 1:12, less than 1:13, less than 1:14, less than 1:15, less than 1:16, less than 1:17, less than 1:18, less than 1:19, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000. In some embodiments that may be combined with any of the preceding embodiments, the population of isolated CD45⁺ cells comprises a ratio of HSPC to Tmem that is approximately 500:1 to approximately 1:1,000. In some embodiments that may be combined with any of the preceding embodiments, the population of isolated CD45⁺ cells comprises a ratio of HSPC to Treg that is from approximately 100:1 to approximately 1:30. In some embodiments that may be combined with any of the preceding embodiments, the population of isolated CD45⁺ cells comprises a ratio of Tmem to Treg that is from approximately 2000:1 to approximately 1:10. In some embodiments that may be combined with any of the preceding embodiments, the population of isolated CD45⁺ cells further comprises an enriched population of TCR Vα24Jα18⁺CD127⁺ invariant Natural Killer T cells (iNKT). In some embodiments that may be combined with any of the preceding embodiments, the population of isolated CD45⁺ cells comprises a dose of approximately 5.0×10² to approximately 2.0×10⁶ iNKT per kilogram of body weight of the human subject receiving the product or a total dose of approximately 2.0×10³ to approximately 4.0×10⁸ iNKT. In some embodiments that may be combined with any of the preceding embodiments, the dose of iNKT comprises a dose of approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product. In some embodiments that may be combined with any of the preceding embodiments, the dose of iNKT comprises a total dose of approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT. In some embodiments that may be combined with any of the preceding embodiments, the population of isolated CD45⁺ cells comprises a ratio of HSPC to iNKT that is from approximately 1:2 to approximately 500,000:1. In some embodiments that may be combined with any of the preceding embodiments, the population of isolated CD45⁺ cells comprises a ratio of iNKT to Tcon is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100. In some embodiments that may be combined with any of the preceding embodiments, the population of isolated CD45⁺ cells comprises a ratio of iNKT to Tmem that is from approximately 5:1 to approximately 1:1,000,000. In some embodiments that may be combined with any of the preceding embodiments, the HSPC are cKIT⁺, CD133⁺, CD90⁺, CD38⁻, CD45RA⁻, Lin⁻, CD19⁻, TCRα⁻, or any combination thereof. In some embodiments that may be combined with any of the preceding embodiments, the Tmem are CD3⁺CD45RO⁺. In some embodiments that may be combined with any of the preceding embodiments, the Tmem comprise a population of T central memory cells (T_{CM}), a population of T effector memory cells (T_{EM}), a population of T stem central memory cells (T_{SCM}), or any combination thereof. In some embodiments that may be combined with any of the preceding embodiments, the T_{CM} are CD4⁺CD45RO⁺ or CD8⁺CD45RO⁺. In some embodiments that may be combined with any of the preceding embodiments, the T_{CM} are CD45RA⁻, CD62L⁺, CCR7⁺, or any combination thereof. In some embodiments that may be combined with any of the preceding embodiments, the T_{EM} are CD4⁺, CD45RO⁺, CD45RA⁻, CD62L⁻, CCR7⁻, or any combination thereof. In some embodiments that may be combined with any of the preceding embodiments, the T_{SCM} are CD4⁺CD45RA⁺ or CD8⁺CD45RA⁺. In some embodiments that may be combined with any of the preceding embodiments, the T_{SCM} are CD95⁺, CD122⁺, CXCR3⁺, LFA-1⁺, or any combination thereof. In some embodiments that may be combined with any of the preceding embodiments, the Treg are FoxP3⁺. In some embodiments that may be combined with any of the preceding embodiments, the Treg comprise a population of naïve Treg cells, a population of memory Treg cells, or a population of naïve Treg cells and memory Treg cells. In some embodiments that may be combined with any of the preceding embodiments, the naïve Treg cells are CD4⁺CD25⁺CD127^{dim}FoxP3⁺CD45RA⁺CD45RO⁻. In some embodiments that may be combined with any of the preceding embodiments, the dose of Treg comprises a dose of approximately 2.0×10⁵ to approximately 5.0×10⁸ naïve Treg cells per kilogram of body weight of the human subject receiving the product, or a total dose of approximately 8.0×10⁵ to approximately 1.0×10¹¹ naïve Treg cells. In some embodiments that may be combined with any of the preceding embodiments, the population of isolated CD45⁺ cells comprises a ratio of HSPC to naïve Treg that is from approximately 1:500 to approximately 100:1. In some embodiments that may be combined with any of the preceding embodiments, the population of isolated CD45⁺ cells comprises a ratio of Tmem to naïve Treg that is from approximately 3:1 to approximately 1:10. In some embodiments that may be combined with any of the preceding embodiments, the population of isolated CD45⁺ cells comprises a ratio of Tcon to naïve Treg that is less than 1:1, less than 1:2, less than 1:3, less than 1:4, less than 1:5, less than 1:6, less than 1:7, less than 1:8, less than 1:9, or less than 1:10. In some embodiments that may be combined with any of the preceding embodiments, the memory Treg cells are CD4⁺CD25⁺CD127^{dim}FoxP3⁺CD45RA⁻CD45RO⁺. In some embodiments that may be combined with any of the preceding embodiments, the dose of Treg comprises a dose of approximately 5.0×10⁴ to approximately 5.0×10⁸ memory Treg cells per kilogram of body weight of the human subject receiving the product, or a total dose of approximately 2.0×10⁵ to approximately 1.0×10¹¹ memory Treg cells. In some embodiments that may be combined with any of the preceding embodiments, the population of isolated CD45⁺ cells comprises a ratio of HSPC to memory Treg that is from approximately 1:500 to approximately 10,000:1. In some embodiments that may be combined with any of the preceding embodiments, the population of isolated CD45⁺ cells comprises a ratio of Tmem to memory Treg that is from approximately 27:1 to approximately 9:10. In some embodiments that may be combined with any of the preceding embodiments, the population of isolated CD45⁺ cells comprises a ratio of Tcon to memory Treg is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100. In some embodiments that may be combined with any of the preceding embodiments, the Tcon are TCRα⁺, TCRβ⁺, or TCRα⁺TCRβ⁺. In some embodiments that may be combined with any of the preceding embodiments, the Tcon are CD25⁻, CD127⁺, or CD25⁻CD127⁺. In some embodiments that may be combined with any of the preceding embodiments, the Tcon are TCRα⁺TCRβ⁺CD45RA⁺CD45RO⁻CD25⁻CD95-IL-2Rβ⁻CD127⁺. In some embodiments that may be combined with any of the preceding embodiments, the iNKT are CD1d-tet⁺, 6B11⁺, or CD1d-tet⁺B11⁺. In some embodiments that may be combined with any of the preceding embodiments, the product further comprises a pharmaceutical composition comprising one or more doses of a graft vs host disease (GVHD) prophylactic agent. In some embodiments that may be combined with any of the preceding embodiments, the GVHD prophylactic agent is tacrolimus. In some embodiments that may be combined with any of the preceding embodiments, one or more doses of tacrolimus are provided to maintain a trough blood level of approximately 5 ng/mL to approximately 10 ng/mL in the human subject receiving the product. In some embodiments that may be combined with any of the preceding embodiments, the pharmaceutical composition comprises tacrolimus at a dose that ranges from approximately 0.01 mg per kilogram of body weight of the human subject receiving the product to approximately 0.50 mg per kilogram of body weight of the human subject receiving the product twice per day. In some embodiments that may be combined with any of the preceding embodiments, the tacrolimus is dosed in an amount to maintain or that maintains a target blood level of approximately 1 ng/mL to approximately 10 ng/mL in the human subject receiving the product for approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the population of isolated CD45⁺ cells. In some embodiments that may be combined with any of the preceding embodiments, the population of isolated CD45⁺ cells comprises from less than approximately 5 EU to less than approximately 0.5 EU of endotoxins per mL of formulation.

In some embodiments that may be combined with any of the preceding embodiments, the HSPC, the Tmem, the Treg, or any combination thereof are obtained from an allogeneic HLA-matched donor, relative to the human subject receiving the product. In some embodiments that may be combined with any of the preceding embodiments, the population of isolated CD45⁺ cells is from an allogeneic HLA-matched donor, relative to the human subject receiving the product. In some embodiments that may be combined with any of the preceding embodiments, the HSPC, the Tmem, the Treg, or any combination thereof are obtained from an allogeneic donor having at least one HLA mismatch, relative to the human subject receiving the product. In some embodiments that may be combined with any of the preceding embodiments, the population of isolated CD45⁺ cells is from an allogeneic donor having at least one HLA mismatch, relative to the human subject receiving the product. In some embodiments that may be combined with any of the preceding embodiments, the at least one HLA mismatch is at an allele selected from: HLA-A, HLA-B, HLA-C, HLA-DRB1, and any combination thereof. In some embodiments that may be combined with any of the preceding embodiments, the cells having at least one HLA mismatch are from a donor that is 6/8 HLA-mismatched, relative to the human subject receiving the product, or is 7/8 HLA-mismatched, relative to the human subject receiving the product.

In some embodiments that may be combined with any of the preceding embodiments, the cells having at least one HLA mismatch are from a donor that is 7/8 HLA-mismatched, relative to the human subject receiving the product. In some embodiments that may be combined with any of the preceding embodiments, the donor that is 7/8 HLA-mismatched, relative to the human subject receiving the product, has a mismatch in HLA-A. In some embodiments that may be combined with any of the preceding embodiments, the donor that is 7/8 HLA-mismatched, relative to the human subject receiving the product, has a mismatch in HLA-B. In some embodiments that may be combined with any of the preceding embodiments, the donor that is 7/8 HLA-mismatched, relative to the human subject receiving the product, has a mismatch in HLA-C. In some embodiments that may be combined with any of the preceding embodiments, the donor that is 7/8 HLA-mismatched, relative to the human subject receiving the product, has a mismatch in HLA-DRB1. In some embodiments that may be combined with any of the preceding embodiments, the donor that has the at least one HLA mismatch, relative to the human subject receiving the product, has a mismatched HLA allele as a result of the donor being homozygous for the HLA allele while the human subject receiving the product is heterogeneous for the HLA allele. In some embodiments that may be combined with any of the preceding embodiments, the donor that has at least one HLA mismatch, relative to the human subject receiving the product, has a mismatched HLA allele as a result of the donor being heterozygous for the HLA allele while the human subject receiving the product is homozygous for the HLA allele. In some embodiments that may be combined with any of the preceding embodiments, the donor that has at least one HLA mismatch, relative to the human subject receiving the product, has a mismatched HLA allele as a result of both the donor and the human subject receiving the product being heterozygous for the HLA allele. In some embodiments that may be combined with any of the preceding embodiments, the HSPC, the Tmem, the Treg, or any combination thereof are obtained from an allogeneic haploidentical donor, relative to the human subject receiving the product. In some embodiments that may be combined with any of the preceding embodiments, the population of isolated CD45⁺ cells is from an allogeneic haploidentical donor, relative to the human subject receiving the product. In some embodiments that may be combined with any of the preceding embodiments, the allogeneic donor is related to the human subject receiving the product. In some embodiments that may be combined with any of the preceding embodiments, the allogeneic donor is unrelated to the human subject receiving the product. In some embodiments that may be combined with any of the preceding embodiments, the body weight of the human subject receiving the product is actual body weight. In some embodiments that may be combined with any of the preceding embodiments, the body weight of the human subject receiving the product is ideal body weight. In some embodiments, the cellular therapy product of any one of the preceding embodiments, or any combination thereof, is for use in a method of treating a human subject having or suspected of having a hematologic malignancy. In some embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN). In some embodiments, the cellular therapy product of any one of the preceding embodiments, or any combination thereof, is for use in a method of treating a human subject having or suspected of having an autoimmune disorder. In some embodiments, the autoimmune disorder is multiple sclerosis.

Other aspects of the present disclosure relate to a method of treating a human subject having or suspected of having acute leukemia in complete remission, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having acute leukemia in complete remission, the method comprising administering to the human subject a cellular therapy product of the present disclosure. In some embodiments that may be combined with any of the preceding embodiments, the human subject has approximately 5% or less blast burden in their bone marrow. In some embodiments that may be combined with any of the preceding embodiments, there is an absence of circulating blasts and/or blasts with Auer rods in the human subject. In some embodiments that may be combined with any of the preceding embodiments, there is an absence of extramedullary disease in the human subject. In some embodiments that may be combined with any of the preceding embodiments, the human subject has an absolute neutrophil count (ANC) that is approximately 1.0×10⁹/L or higher. In some embodiments that may be combined with any of the preceding embodiments, the human subject has a platelet count that is approximately 1.0×10¹¹/L or higher. In some embodiments that may be combined with any of the preceding embodiments, the human subject is red blood cell-transfusion-independent. In some embodiments that may be combined with any of the preceding embodiments, the acute leukemia is in complete remission with incomplete hematologic recovery. In some embodiments that may be combined with any of the preceding embodiments, the human subject has residual neutropenia. In some embodiments that may be combined with any of the preceding embodiments, the human subject has an absolute neutrophil count (ANC) that is approximately 1.0×10⁹/L or lower. In some embodiments that may be combined with any of the preceding embodiments, the human subject has residual thrombocytopenia. In some embodiments that may be combined with any of the preceding embodiments, the human subject has a platelet count that is approximately 1.0×10¹¹/L or lower.

Other aspects of the present disclosure relate to a method of treating a human subject having or suspected of having active acute leukemia, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁶ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having active acute leukemia, the method comprising administering to the human subject a cellular therapy product of the present disclosure. In some embodiments that may be combined with any of the preceding embodiments, the human subject has approximately 10% or less leukemic blast infiltration of bone marrow. In some embodiments that may be combined with any of the preceding embodiments, the acute leukemia is acute myeloid leukemia (AML), acute lymphoid leukemia (ALL), and/or mixed phenotype acute leukemia (MPAL).

Other aspects of the present disclosure relate to a method of treating a human subject having or suspected of having chronic myelogenous leukemia (CML), the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having chronic myelogenous leukemia (CML), the method comprising administering to the human subject a cellular therapy product of the present disclosure. In some embodiments that may be combined with any of the preceding embodiments, the human subject is in myeloid blast crises and/or lymphoid blast crisis. In some embodiments that may be combined with any of the preceding embodiments, the blast crisis is in complete remission. In some embodiments that may be combined with any of the preceding embodiments, the blast crisis is in complete remission with incomplete hematologic recovery. In some embodiments that may be combined with any of the preceding embodiments, the CML is in accelerated phase. In some embodiments that may be combined with any of the preceding embodiments, the CML is in chronic phase. In some embodiments that may be combined with any of the preceding embodiments, the chronic phase CML is resistant to or intolerant of one or more first-generation or second-generation tyrosine kinase inhibitors (TKIs).

Other aspects of the present disclosure relate to a method of treating a human subject having or suspected of having high-risk or very high-risk myelodysplastic syndrome (MDS), the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having high-risk or very high-risk myelodysplastic syndrome (MDS), the method comprising administering to the human subject a cellular therapy product of the present disclosure. In some embodiments, the human subject has approximately 10% or less blast burden in their bone marrow.

Other aspects of the present disclosure relate to a method of treating a human subject having or suspected of having myelofibrosis, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having myelofibrosis, the method comprising administering to the human subject a cellular therapy product of the present disclosure. In some embodiments that may be combined with any of the preceding embodiments, the myelofibrosis is eligible for standard myeloablative allogeneic hematopoietic stem cell transplant (alloHSCT). In some embodiments that may be combined with any of the preceding embodiments, the myelofibrosis is intermediate-2-risk myelofibrosis or high-risk myelofibrosis. In some embodiments that may be combined with any of the preceding embodiments, the myelofibrosis is intermediate-1-risk myelofibrosis associated with high symptom burden, low platelet counts, and/or complex cytogenetics.

Other aspects of the present disclosure relate to a method of treating a human subject having or suspected of having a hematologic malignancy, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon).

In some embodiments that may be combined with any of the preceding embodiments, the cellular therapy product comprises a cellular therapy product of the present disclosure. In some embodiments that may be combined with any of the preceding embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN). In some embodiments that may be combined with any of the preceding embodiments, the method further comprises administering one or more doses of a graft vs host disease (GVHD) prophylactic agent. In some embodiments that may be combined with any of the preceding embodiments, the GVHS prophylactic agent is tacrolimus. In some embodiments that may be combined with any of the preceding embodiments, the tacrolimus is provided in an amount sufficient to maintain a trough blood level of approximately 5 ng/mL to approximately 10 ng/mL in the human subject. In some embodiments that may be combined with any of the preceding embodiments, the tacrolimus is provided at a dose that ranges from approximately 0.01 mg per kilogram of body weight of the human subject to 0.50 mg per kilogram of body weight of the human subject twice per day. In some embodiments that may be combined with any of the preceding embodiments, the tacrolimus is dosed in an amount to maintain or that maintains a target blood level of approximately 1 ng/mL to approximately 10 ng/mL in the human subject for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45⁺ cells. In some embodiments that may be combined with any of the preceding embodiments, the tacrolimus is initially administered from approximately 12 hours to approximately 24 hours after administration of the cellular therapy product. In some embodiments that may be combined with any of the preceding embodiments, administration of the tacrolimus is tapered starting at approximately 90 days after initial administration of the tacrolimus. In some embodiments that may be combined with any of the preceding embodiments, the administering comprises infusing into the human subject the population of isolated CD45⁺ cells. In some embodiments that may be combined with any of the preceding embodiments, the population of isolated CD45⁺ cells comprises from approximately 1.0×10⁵ to approximately 1.0×10⁸ HSPC per kilogram of body weight of the human subject, or from approximately 5.0×10⁵ to approximately 1.5×10¹⁰ HSPC. In some embodiments that may be combined with any of the preceding embodiments, the population of isolated CD45⁺ cells comprises approximately 1.0×10⁵ or more HSPC per kilogram of body weight of the human subject, approximately 2.0×10⁵ or more HSPC per kilogram of body weight of the human subject, approximately 3.0×10⁵ or more HSPC per kilogram of body weight of the human subject, approximately 4.0×10⁵ or more HSPC per kilogram of body weight of the human subject, approximately 5.0×10⁵ or more HSPC per kilogram of body weight of the human subject, approximately 6.0×10⁵ or more HSPC per kilogram of body weight of the human subject, approximately 7.0×10⁵ or more HSPC per kilogram of body weight of the human subject, approximately 8.0×10⁵ or more HSPC per kilogram of body weight of the human subject, approximately 9.0×10⁵ or more HSPC per kilogram of body weight of the human subject, approximately 1.0×10⁶ or more HSPC per kilogram of body weight of the human subject, approximately 1.5×10⁶ or more HSPC per kilogram of body weight of the human subject, approximately 2.0×10⁶ or more HSPC per kilogram of body weight of the human subject, approximately 2.5×10⁶ or more HSPC per kilogram of body weight of the human subject, approximately 3.0×10⁶ or more HSPC per kilogram of body weight of the human subject, approximately 3.5×10⁶ or more HSPC per kilogram of body weight of the human subject, approximately 4.0×10⁶ or more HSPC per kilogram of body weight of the human subject, approximately 4.5×10⁶ or more HSPC per kilogram of body weight of the human subject, approximately 5.0×10⁶ or more HSPC per kilogram of body weight of the human subject, approximately 5.5×10⁶ or more HSPC per kilogram of body weight of the human subject, approximately 6.0×10⁶ or more HSPC per kilogram of body weight of the human subject, approximately 6.5×10⁶ or more HSPC per kilogram of body weight of the human subject, approximately 7.0×10⁶ or more HSPC per kilogram of body weight of the human subject, approximately 7.5×10⁶ or more HSPC per kilogram of body weight of the human subject, approximately 8.0×10⁶ or more HSPC per kilogram of body weight of the human subject, approximately 8.5×10⁶ or more HSPC per kilogram of body weight of the human subject, approximately 9.0×10⁶ or more HSPC per kilogram of body weight of the human subject, approximately 9.5×10⁶ or more HSPC per kilogram of body weight of the human subject, approximately 1.0×10⁷ or more HSPC per kilogram of body weight of the human subject, approximately 1.5×10⁷ or more HSPC per kilogram of body weight of the human subject, approximately 2.0×10⁷ or more HSPC per kilogram of body weight of the human subject, approximately 2.5×10⁷ or more HSPC per kilogram of body weight of the human subject, approximately 3.0×10⁷ or more HSPC per kilogram of body weight of the human subject, approximately 3.5×10⁷ or more HSPC per kilogram of body weight of the human subject, approximately 4.0×10⁷ or more HSPC per kilogram of body weight of the human subject, approximately 4.5×10⁷ or more HSPC per kilogram of body weight of the human subject, approximately 5.0×10⁷ or more HSPC per kilogram of body weight of the human subject, approximately 5.5×10⁷ or more HSPC per kilogram of body weight of the human subject, approximately 6.0×10⁷ or more HSPC per kilogram of body weight of the human subject, approximately 6.5×10⁷ or more HSPC per kilogram of body weight of the human subject, approximately 7.0×10⁷ or more HSPC per kilogram of body weight of the human subject, approximately 7.5×10⁷ or more HSPC per kilogram of body weight of the human subject, approximately 8.0×10⁷ or more HSPC per kilogram of body weight of the human subject, approximately 8.5×10⁷ or more HSPC per kilogram of body weight of the human subject, approximately 9.0×10⁷ or more HSPC per kilogram of body weight of the human subject, approximately 9.5×10⁷ or more HSPC per kilogram of body weight of the human subject, or approximately 1.0×10⁸ or more HSPC per kilogram of body weight of the human subject. In some embodiments that may be combined with any of the preceding embodiments, the population of isolated CD45⁺ cells comprises approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC. In some embodiments that may be combined with any of the preceding embodiments, the population of isolated CD45⁺ cells comprises from approximately 1.0×10⁵ to approximately 1.0×10⁸ Tmem per kilogram of body weight of the human subject receiving the product from approximately 5.0×10⁵ to approximately 1.5×10¹⁰ Tmem. In some embodiments that may be combined with any of the preceding embodiments, the population of isolated CD45⁺ cells comprises approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product. In some embodiments that may be combined with any of the preceding embodiments, the population of isolated CD45⁺ cells comprises approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem. In some embodiments that may be combined with any of the preceding embodiments, the population of isolated CD45⁺ cells comprises from approximately 1.0×10⁵ to approximately 2.0×10⁷ fresh Treg per kilogram of body weight of the human subject or from approximately 5.0×10⁵ to approximately 3.0×10⁹ fresh Treg. In some embodiments that may be combined with any of the preceding embodiments, the population of isolated CD45⁺ cells comprises approximately 1.0×10⁵ or more fresh Treg per kilogram of body weight of the human subject, approximately 2.0×10⁵ or more fresh Treg per kilogram of body weight of the human subject, approximately 3.0×10⁵ or more fresh Treg per kilogram of body weight of the human subject, approximately 4.0×10⁵ or more fresh Treg per kilogram of body weight of the human subject, approximately 5.0×10⁵ or more fresh Treg per kilogram of body weight of the human subject, approximately 6.0×10⁵ or more fresh Treg per kilogram of body weight of the human subject, approximately 7.0×10⁵ or more fresh Treg per kilogram of body weight of the human subject, approximately 8.0×10⁵ or more fresh Treg per kilogram of body weight of the human subject, approximately 9.0×10⁵ or more fresh Treg per kilogram of body weight of the human subject, approximately 1.0×10⁶ or more fresh Treg per kilogram of body weight of the human subject, approximately 1.5×10⁶ or more fresh Treg per kilogram of body weight of the human subject, approximately 2.0×10⁶ or more fresh Treg per kilogram of body weight of the human subject, approximately 2.5×10⁶ or more fresh Treg per kilogram of body weight of the human subject, approximately 3.0×10⁶ or more fresh Treg per kilogram of body weight of the human subject, approximately 3.5×10⁶ or more fresh Treg per kilogram of body weight of the human subject, approximately 4.0×10⁶ or more fresh Treg per kilogram of body weight of the human subject, approximately 4.5×10⁶ or more fresh Treg per kilogram of body weight of the human subject, approximately 5.0×10⁶ or more fresh Treg per kilogram of body weight of the human subject, approximately 5.5×10⁶ or more fresh Treg per kilogram of body weight of the human subject, approximately 6.0×10⁶ or more fresh Treg per kilogram of body weight of the human subject, approximately 6.5×10⁶ or more fresh Treg per kilogram of body weight of the human subject, approximately 7.0×10⁶ or more fresh Treg per kilogram of body weight of the human subject, approximately 7.5×10⁶ or more fresh Treg per kilogram of body weight of the human subject, approximately 8.0×10⁶ or more fresh Treg per kilogram of body weight of the human subject, approximately 8.5×10⁶ or more fresh Treg per kilogram of body weight of the human subject, approximately 9.0×10⁶ or more fresh Treg per kilogram of body weight of the human subject, approximately 9.5×10⁶ or more fresh Treg per kilogram of body weight of the human subject, approximately 1.0×10⁷ or more fresh Treg per kilogram of body weight of the human subject, approximately 1.5×10⁷ or more fresh Treg per kilogram of body weight of the human subject, or approximately 2.0×10⁷ or more fresh Treg per kilogram of body weight of the human subject. In some embodiments that may be combined with any of the preceding embodiments, the population of isolated CD45⁺ cells comprises approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg. In some embodiments that may be combined with any of the preceding embodiments, the population of isolated CD45⁺ cells comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product. In some embodiments that may be combined with any of the preceding embodiments, the population of isolated CD45⁺ cells comprises less than approximately 6.0×10⁷ Tcon, less than approximately 5.5×10⁷ Tcon, less than approximately 5.0×10⁷ Tcon, less than approximately 4.5×10⁷ Tcon, less than approximately 4.0×10⁷ Tcon, less than approximately 3.5×10⁷ Tcon, less than approximately 3.0×10⁷ Tcon, less than approximately 2.5×10⁷ Tcon, less than approximately 2.0×10⁷ Tcon, less than approximately 1.5×10⁷ Tcon, less than approximately 1.0×10⁷ Tcon, less than approximately 9.5×10⁶ Tcon, less than approximately 9.0×10⁶ Tcon, less than approximately 8.5×10⁶ Tcon, less than approximately 8.0×10⁶ Tcon, less than approximately 7.5×10⁶ Tcon, less than approximately 7.0×10⁶ Tcon, less than approximately 6.5×10⁶ Tcon, less than approximately 6.0×10⁶ Tcon, less than approximately 5.5×10⁶ Tcon, less than approximately 5.0×10⁶ Tcon, less than approximately 4.5×10⁶ Tcon, less than approximately 4.0×10⁶ Tcon, less than approximately 3.5×10⁶ Tcon, less than approximately 3.0×10⁶ Tcon, less than approximately 2.5×10⁶ Tcon, less than approximately 2.0×10⁶ Tcon, less than approximately 1.5×10⁶ Tcon, or less than approximately 1.0×10⁶ Tcon. In some embodiments that may be combined with any of the preceding embodiments, the population of isolated CD45⁺ cells further comprises an enriched population of TCR Vα24Jα18⁺CD127⁺ invariant Natural Killer T cells (iNKT). In some embodiments that may be combined with any of the preceding embodiments, the population of isolated CD45⁺ cells comprises from approximately 5.0×10² to approximately 2.0×10⁶ iNKT per kilogram of body weight of the human subject receiving the product or from approximately 2.0×10³ to approximately 4.0×10⁸ iNKT. In some embodiments that may be combined with any of the preceding embodiments, the population of isolated CD45⁺ cells comprises approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product. In some embodiments that may be combined with any of the preceding embodiments, the population of isolated CD45⁺ cells comprises approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT. In some embodiments that may be combined with any of the preceding embodiments, the population of isolated CD45⁺ cells is from an allogeneic donor having at least one HLA mismatch relative to the human subject. In some embodiments that may be combined with any of the preceding embodiments, the HSPC are cKIT⁺, CD133⁺, CD90⁺, CD38⁻, CD45RA⁻, Lin⁻, CD19⁻, TCRα⁻, or any combination thereof. In some embodiments that may be combined with any of the preceding embodiments, the Tmem are CD3⁺CD45RO⁺. In some embodiments that may be combined with any of the preceding embodiments, the Tmem comprise a population of T central memory cells (T_{CM}), a population of T effector memory cells (T_{EM}), a population of T stem central memory cells (T_{SCM}), or any combination thereof. In some embodiments that may be combined with any of the preceding embodiments, the T_{CM} are CD4⁺CD45RO⁺ or CD8⁺CD45RO⁺. In some embodiments that may be combined with any of the preceding embodiments, the T_{CM} are CD45RA⁻, CD62L⁺, CCR7⁺, or any combination thereof. In some embodiments that may be combined with any of the preceding embodiments, the T_{EM} are CD4⁺, CD45RO⁺, CD45RA⁻, CD62L⁻, CCR7⁻, or any combination thereof. In some embodiments that may be combined with any of the preceding embodiments, the T_{SCM} are CD4⁺CD45RA⁺ or CD8⁺CD45RA⁺. In some embodiments that may be combined with any of the preceding embodiments, the T_{SCM} are CD95⁺, CD122⁺, CXCR3⁺, LFA-1+, or any combination thereof. In some embodiments that may be combined with any of the preceding embodiments, the Treg are FOXP3⁺. In some embodiments that may be combined with any of the preceding embodiments, the Treg comprise a population of naïve Treg cells, a population of memory Treg cells, or a population of naïve Treg cells and memory Treg cells. In some embodiments that may be combined with any of the preceding embodiments, the naïve Treg cells are CD4⁺CD25⁺CD127^{dim}FoxP3⁺CD45RA⁺CD45RO⁻. In some embodiments that may be combined with any of the preceding embodiments, the Treg comprise from approximately 2.0×10⁵ to approximately 5.0×10⁸ naïve Treg cells per kilogram of body weight of the human subject receiving the product, or from approximately 8.0×10⁵ to approximately 1.0×10¹¹ naïve Treg cells. In some embodiments that may be combined with any of the preceding embodiments, the memory Treg cells are CD4⁺CD25⁺CD127^{dim}FoxP3⁺CD45RA-CD45RO⁺. In some embodiments that may be combined with any of the preceding embodiments, the Treg comprise from approximately 5.0×10⁴ to approximately 5.0×10⁸ memory Treg cells per kilogram of body weight of the human subject receiving the product, or from approximately 2.0×10⁵ to approximately 1.0×10¹¹ memory Treg cells. In some embodiments that may be combined with any of the preceding embodiments, the Tcon are TCRα⁺, TCRβ⁺, or TCRα⁺TCRβ⁺. In some embodiments that may be combined with any of the preceding embodiments, the Tcon are CD25⁻, CD127+, or CD25⁻CD127⁺. In some embodiments that may be combined with any of the preceding embodiments, the Tcon are TCRα⁺TCRβ⁺CD45RA⁺CD45RO⁻CD25⁻CD95⁻IL-2RO⁻CD127+. In some embodiments that may be combined with any of the preceding embodiments, the iNKT are CD1d-tet⁺, 6B11, or CD1d-tet⁺6B11⁺. In some embodiments that may be combined with any of the preceding embodiments, the population of isolated CD45⁺ cells comprises from less than approximately 5 EU to less than approximately 0.5 EU of endotoxins per mL of formulation.

In some embodiments that may be combined with any of the preceding embodiments, the HSPC, the Tmem, the Treg, or any combination thereof are obtained from an allogeneic HLA-matched donor relative to the human subject receiving the product. In some embodiments that may be combined with any of the preceding embodiments, the population of isolated CD45⁺ cells is from an allogeneic HLA-matched donor relative to the human subject receiving the product. In some embodiments that may be combined with any of the preceding embodiments, the HSPC, the Tmem, the Treg, or any combination thereof are obtained from an allogeneic donor having at least one HLA mismatch relative to the human subject receiving the product. In some embodiments that may be combined with any of the preceding embodiments, the population of isolated CD45⁺ cells is from an allogeneic donor having at least one HLA mismatch relative to the human subject receiving the product. In some embodiments that may be combined with any of the preceding embodiments, the at least one HLA mismatch is at an allele selected from: HLA-A, HLA-B, HLA-C, HLA-DRB1, and any combination thereof. In some embodiments that may be combined with any of the preceding embodiments, the cells having at least one HLA mismatch are from a donor that is 6/8 HLA-mismatched relative to the human subject receiving the product or is 7/8 HLA-mismatched relative to the human subject receiving the product. In some embodiments that may be combined with any of the preceding embodiments, the cells having at least one HLA mismatch are from a donor that is 7/8 HLA-mismatched relative to the human subject receiving the product. In some embodiments that may be combined with any of the preceding embodiments, the donor that is 7/8 HLA-mismatched relative to the human subject receiving the product has a mismatch in HLA-A. In some embodiments that may be combined with any of the preceding embodiments, the donor that is 7/8 HLA-mismatched relative to the human subject receiving the product has a mismatch in HLA-B. In some embodiments that may be combined with any of the preceding embodiments, the donor that is 7/8 HLA-mismatched relative to the human subject receiving the product has a mismatch in HLA-C. In some embodiments that may be combined with any of the preceding embodiments, the donor that is 7/8 HLA-mismatched relative to the human subject receiving the product has a mismatch in HLA-DRB1. In some embodiments that may be combined with any of the preceding embodiments, the donor that has the at least one HLA mismatch relative to the human subject receiving the product has a mismatched HLA allele as a result of the donor being homozygous for the HLA allele while the human subject receiving the product is heterogeneous for the HLA allele. In some embodiments that may be combined with any of the preceding embodiments, the donor that has at least one HLA mismatch relative to the human subject receiving the product has a mismatched HLA allele as a result of the donor being heterozygous for the HLA allele while the human subject receiving the product is homozygous for the HLA allele. In some embodiments that may be combined with any of the preceding embodiments, the donor that has at least one HLA mismatch relative to the human subject receiving the product has a mismatched HLA allele as a result of both the donor and the human subject receiving the product being heterozygous for the HLA allele. In some embodiments that may be combined with any of the preceding embodiments, the HSPC, the Tmem, the Treg, or any combination thereof are obtained from an allogeneic haploidentical donor relative to the human subject receiving the product. In some embodiments that may be combined with any of the preceding embodiments, the population of isolated CD45⁺ cells is from an allogeneic haploidentical donor relative to the human subject receiving the product. In some embodiments that may be combined with any of the preceding embodiments, the allogeneic donor is related to the human subject receiving the product. In some embodiments that may be combined with any of the preceding embodiments, the allogeneic donor is unrelated to the human subject receiving the product.

In some embodiments that may be combined with any of the preceding embodiments, the method further comprises collecting one or more mobilized peripheral blood donations from the donor. In some embodiments that may be combined with any of the preceding embodiments, the peripheral blood donations are mobilized by granulocyte colony-stimulating factor (G-CSF), granulocyte macrophage colony-stimulating factor (GM-CSF), plerixafor, or any combination thereof. In some embodiments that may be combined with any of the preceding embodiments, at least one of the mobilized peripheral blood donations is processed and sorted using one or more immune-separation particles (ISPs) to enrich HSPC, Tmem, and Treg. In some embodiments that may be combined with any of the preceding embodiments, the one or more ISPs comprise affinity reagents. In some embodiments that may be combined with any of the preceding embodiments, the affinity reagents are immuno-magnetic separation particles. In some embodiments that may be combined with any of the preceding embodiments, the immuno-magnetic separation particles are antibodies each conjugated to an iron-containing particle. In some embodiments that may be combined with any of the preceding embodiments, the affinity reagents comprise a plurality of CD34-reagents that binds to CD34 on an HSPC. In some embodiments that may be combined with any of the preceding embodiments, an average number of ISP's per HSPC in the population of isolated CD45⁺ cells is equal to or less than approximately 20,000. In some embodiments that may be combined with any of the preceding embodiments, an average number of ISP's per HSPC in the population of isolated CD45⁺ cells is from approximately 1000 to approximately 20,000. In some embodiments that may be combined with any of the preceding embodiments, the affinity reagents comprise a plurality of CD45RA-reagents that binds to one or more CD45RA receptors on a Tmem. In some embodiments that may be combined with any of the preceding embodiments, the affinity reagents comprise a plurality of CD25-reagents that binds to one or more CD25 receptors on a Treg. In some embodiments that may be combined with any of the preceding embodiments, an average number of ISPs per Treg in the population of isolated CD45⁺ cells is equal or less than approximately 4000. In some embodiments that may be combined with any of the preceding embodiments, an average number of ISPs per Treg in the population of isolated CD45⁺ cells is from approximately 1500 to approximately 2500. In some embodiments that may be combined with any of the preceding embodiments, the method further comprises administering a conditioning regimen prior to administration of the cellular therapy product. In some embodiments that may be combined with any of the preceding embodiments, the conditioning regimen is administered from approximately two days to approximately ten days before administration of the multi-component cellular therapy. In some embodiments that may be combined with any of the preceding embodiments, the conditioning regimen is a total body irradiation-based (TBI-based) regimen or a total lymphatic irradiation-based (TLI-based) regimen. In some embodiments that may be combined with any of the preceding embodiments, the TBI-based regimen comprises fractionated total body irradiation. In some embodiments that may be combined with any of the preceding embodiments, the fractionated total body irradiation comprises 2 or more, 3 or more, 4 or more, 5 or more, 6 or more, 7 or more, 8 or more, 9 or more, 10 or more, 11 or more, 12 or more, 13 or more, 14 or more, or 15 or more doses. In some embodiments that may be combined with any of the preceding embodiments, the fractionated total body irradiation comprises a total dose that ranges from approximately 500 to approximately 1600 cGy. In some embodiments that may be combined with any of the preceding embodiments, the TBI-based regimen further comprises one or more conditioning reagents. In some embodiments that may be combined with any of the preceding embodiments, the one or more conditioning reagents are selected from: cyclophosphamide, etoposide, fludarabine, thiotepa, anti-thymocyte globulin (ATG), and any combination thereof. In some embodiments that may be combined with any of the preceding embodiments, the conditioning regimen is a myeloablative conditioning regimen. In some embodiments that may be combined with any of the preceding embodiments, the myeloablative conditioning regimen comprises one or more conditioning reagents. In some embodiments that may be combined with any of the preceding embodiments, the one or more conditioning reagents are selected from: thiotepa, busulfan, melphalan, fludarabine, cyclophosphamide, anti-thymocyte globulin (ATG), and any combination thereof. In some embodiments that may be combined with any of the preceding embodiments, the myeloablative conditioning regimen comprises three or more conditioning reagents, wherein at least one conditioning reagent comprises thiotepa. In some embodiments that may be combined with any of the preceding embodiments, the myeloablative conditioning regimen comprises one or more doses of busulfan, fludarabine, and thiotepa. In some embodiments that may be combined with any of the preceding embodiments, the one or more doses of busulfan, fludarabine, and thiotepa comprise from approximately 5 to approximately 12 mg of thiotepa per kilogram of body weight of the human subject, from approximately 7 to approximately 11 mg of busulfan per kilogram of body weight of the human subject, and from approximately 100 to approximately 200 mg of fludarabine per meter² body surface area respectively.

In some embodiments that may be combined with any of the preceding embodiments, overall survival rate 12 months or more after administration of the cellular therapy product ranges from approximately 75% to approximately 100%. In some embodiments that may be combined with any of the preceding embodiments, overall survival of the human subject increases after administration of the cellular therapy product, relative to a corresponding human subject that has been administered a standard myeloablative allogeneic hematopoietic stem cell transplant (alloHSCT). In some embodiments that may be combined with any of the preceding embodiments, GVHD and relapse-free survival (GFRS) rate12 months or more after administration of the cellular therapy product ranges from approximately 65% to approximately 100%. In some embodiments that may be combined with any of the preceding embodiments, GVHD and relapse-free survival (GFRS) of the human subject increases after administration of the cellular therapy product, relative to a corresponding human subject that has been administered a standard myeloablative allogeneic hematopoietic stem cell transplant (alloHSCT). In some embodiments that may be combined with any of the preceding embodiments, relapse-free survival rate12 months or more after administration of the cellular therapy product ranges from approximately 70% to approximately 100%. In some embodiments that may be combined with any of the preceding embodiments, relapse-free survival of the human subject increases after administration of the cellular therapy product, relative to a corresponding human subject that has been administered a standard myeloablative allogeneic hematopoietic stem cell transplant (alloHSCT). In some embodiments that may be combined with any of the preceding embodiments, incidence of acute GVHD 100 or more days after administration of the cellular therapy product ranges from approximately 9% to approximately 31%. In some embodiments that may be combined with any of the preceding embodiments, incidence of chronic GVHD 100 or more days after administration of the cellular therapy product ranges from approximately 1% to approximately 10%. In some embodiments that may be combined with any of the preceding embodiments, incidence of post-transplant lymphoproliferative disorder (PTLD) 100 or more days after administration of the cellular therapy product ranges from approximately 1% to approximately 10%. In some embodiments that may be combined with any of the preceding embodiments, incidence of Grade 2 or higher infections 100 or more days after administration of the cellular therapy product ranges from approximately 23% to approximately 31%.

Other aspects of the present disclosure relate to a method of treating a human subject having or suspected of having multiple sclerosis, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). Other aspects of the present disclosure relate to a method of treating a human subject having or suspected of having multiple sclerosis, the method comprising administering to the human subject a cellular therapy product of the present disclosure. In some embodiments, the multiple sclerosis is primary progressive multiple sclerosis. In some embodiments, the multiple sclerosis is chronic progressive multiple sclerosis. In some embodiments, the multiple sclerosis is secondary progressive multiple sclerosis. In some embodiments, the multiple sclerosis is relapsing-remitting multiple sclerosis. In some embodiments, the multiple sclerosis is acute relapsing multiple sclerosis.

In some embodiments that may be combined with any of the preceding embodiments, the human subject is approximately 3 months of age or older. In some embodiments that may be combined with any of the preceding embodiments, the human subject is from between approximately 3 months to approximately 18 years of age. In some embodiments that may be combined with any of the preceding embodiments, the human subject is approximately 12 years of age or older. In some embodiments that may be combined with any of the preceding embodiments, the human subject is between approximately 12 years to approximately 65 years of age. In some embodiments that may be combined with any of the preceding embodiments, the human subject is between approximately 12 years to approximately 75 years of age. In some embodiments that may be combined with any of the preceding embodiments, the human subject is from approximately 65 years of age to approximately 75 years of age. In some embodiments that may be combined with any of the preceding embodiments, the human subject is from between approximately 3 months to approximately 75 years of age. In some embodiments that may be combined with any of the preceding embodiments, the human subject has received from one to five previous lines of therapy.

Other aspects of the present disclosure relate to a cellular therapy kit comprising a cellular therapy product of the present disclosure. Other aspects of the present disclosure relate to a cellular therapy kit comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. Other aspects of the present disclosure relate to a cellular therapy kit comprising a container comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. In some embodiments that may be combined with any of the preceding embodiments, the kit further comprises written instructions for using the cellular therapy for treating a hematologic malignancy in a human subject. In some embodiments that may be combined with any of the preceding embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN). In some embodiments that may be combined with any of the preceding embodiments, the kit further comprises written instructions for using the cellular therapy for treating multiple sclerosis in a human subject.

Other aspects of the present disclosure relate to a unit dose comprising a cellular therapy product of the present disclosure. Other aspects of the present disclosure relate to a unit dose of comprising a population of isolated CD45⁺ cells, wherein the population of isolated CD45⁺ cells comprises: a) approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and/or c) approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). In some embodiments, the population of isolated CD45⁺ cells further comprises one or more excipients. Other aspects of the present disclosure relate to a unit dose comprising: a) approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC), and one or more excipients; b) approximately 1.0×10⁵ to approximately 1.0×10⁸ CD45RA-memory T cells (Tmem) per kilogram of body weight of the human subject receiving the product or a total dose of approximately 5.0×10⁵ to approximately 1.5×10¹⁰ CD45RA⁻ memory T cells (Tmem), and one or more excipients; and c) approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), and one or more excipients, wherein the unit dose has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon).

Other aspects of the present disclosure relate to an article of manufacture comprising a cellular therapy product of the present disclosure. Other aspects of the present disclosure relate to an article of manufacture comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. Other aspects of the present disclosure relate to an article of manufacture comprising a container comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. In some embodiments that may be combined with any of the preceding embodiments, the article of manufacture further comprises instructions for administering the article of manufacture to a human subject treat a hematologic malignancy. In some embodiments that may be combined with any of the preceding embodiments, the hematologic malignancy is selected from: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN). In some embodiments that may be combined with any of the preceding embodiments, the article of manufacture further comprises instructions for administering the article of manufacture to a human subject treat multiple sclerosis.

Other aspects of the present disclosure relate to a container comprising a cellular therapy product of the present disclosure. Other aspects of the present disclosure relate to a container comprising a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. Other aspects of the present disclosure relate to a container comprising: a) approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC), and one or more excipients; b) approximately 1.0×10⁵ to approximately 1.0×10⁸ CD45RA-memory T cells (Tmem) per kilogram of body weight of the human subject receiving the product or a total dose of approximately 5.0×10⁵ to approximately 1.5×10¹⁰ CD45RA⁻ memory T cells (Tmem), and one or more excipients; and c) approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), and one or more excipients, wherein the HSPC, Tmem, and/or Treg have been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon).

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a schematic of graft production and administration.

FIGS. 2A-C illustrate neutrophil and platelet engraftment in the study cohorts.

FIGS. 3A-C depict the incidence of grade 2-4 aGVHD in study cohorts (e.g., MRD/MUD, and MMUD in ArmA; Haplo in ArmB; and no-GVHD prophylaxis in ArmC).

FIGS. 4A-C depicts the incidence of grade 2-4 aGVHD in study cohorts that have received over 1×10⁶ Tmem per kg of the recipient subject's ABW or (adjusted) IBW.

FIGS. 5A-C depicts the incidence of grade 3-4 aGVHD in different cohorts of the study.

FIGS. 6A-C depicts the incidence of chronic GVHD of all grades in cohorts of the study.

FIGS. 7A-C depicts the incidence of disease relapse in different cohorts of the study.

FIGS. 8A-C depicts the incidence of NRM in different cohorts of the study.

FIGS. 9A-C depicts the OS in different cohorts of the study.

FIGS. 10A-C depicts the RFS in different cohorts of the study.

FIGS. 11A-C depicts the GRFS in different cohorts of the study.

FIGS. 12A-D depicts the incidence of all infections in various arms of the study at about a year after the transplant.

FIGS. 13A-C depicts the incidence of all infections in various cohorts of the study at about a year after the transplant.

FIGS. 14A-B depicts the incidence of infections in various arms of the study at about 1000 days after the transplant.

FIG. 15 compares the incidence of relapse and study design of the present study to that of Gooptu (Blood 2021) and the Wagner (Blood Advances 2021).

FIG. 16 depicts GVHD relapse free survival in haploidentical transplants.

FIG. 17 depicts overall survival (OS) by combined Tmem dose.

FIG. 18 depicts overall survival (OS) by study arm with a combined Tmem dose of ≥10×10⁶ cells/kg.

FIG. 19 depicts GVHD-free and relapse-free survival (GRFS) by combined Tmem dose.

FIG. 20 depicts GVHD-free and relapse-free survival (GRFS) by study arm with a combined Tmem dose of ≥10×10⁶ cells/kg.

FIG. 21 depicts relapse-free survival by combined Tmem dose.

FIG. 22 depicts relapse-free survival by study arm with a combined Tmem dose of 10×10⁶ cells/kg.

FIG. 23 depicts GVHD relapse free survival (GRFS) in haploidentical transplants.

### DETAILED DESCRIPTION

### Introduction

Various embodiments of the present disclosure provide compositions, pharmaceutical treatments, cell populations, solutions, formulations, kits, and/or methods relating to improved allogeneic hematopoietic stem cell transplantation (alloHCT).

Standard alloHSCT is the transplantation of multipotent hematopoietic stem and progenitor cells (HSPC), usually derived from donor bone marrow, peripheral blood, or umbilical cord blood, into a recipient. The recipient can be subjected to myeloablative conditioning, which kills hematopoietic cells including tumor cells and host immune cells. The HSPC transplanted into the recipient then reconstitutes the hematopoietic compartment. HCT can be useful as a treatment for cancer due to the ability of donor T cells to exert anti-tumor effects, referred to as graft versus tumor (GVT). In patients with hematologic malignancies that are refractory to chemotherapy, HCT is associated with improved survival.

Surprisingly, alloHCT recipients who received a single agent graft versus host disease (GVHD) prophylactic, such as tacrolimus, had significantly better clinical outcomes than existing alloHCT regimens and standards of care. These recipients experience improved clinical outcomes including, for example, increased overall survival, increased relapse-free survival, increased GVHD- and relapse-free survival (GRFS), more rapid and/or complete engraftment of various hematopoietic components (e.g., neutrophils, platelets, T cells, B cells), improved donor chimerism (e.g., T cell chimerism), decreased relapse, decreased primary graft failure, decreased secondary graft failure, decreased treatment-associated mortality, reduced acute and/or chronic GVHD, and shorter time to discharge from hospital following the single agent GVHD prophylactic consisting of tacrolimus

Although alloHCT is associated with improved survival in patients with hematologic malignancies that are refractory to chemotherapy, some subjects treated with existing alloHCT regimens exhibit cancer relapse, and a number of complications can limit the efficacy of standard alloHCT. The effectiveness of standard alloHCT can be limited by, for example, primary graft failure, secondary graft failure, limited or slow engraftment of various hematopoietic components (e.g., neutrophils, platelets, T cells, or B cells), and limited donor chimerism (e.g., T cell chimerism). Additionally, standard alloHCT can cause treatment-associated morality or toxicity, for example, However, donor T cells can also attack non-tumor host cells, resulting in graft versus host disease (GVHD). GVHD is a major source of post-HCT complications and can be fatal. Management of GVHD can require immunosuppressive therapy or cytotoxic mediations, which can cause toxicity, increase susceptibility to infection, and/or blunt anti-tumor immunity. The early morbidity and mortality associated with acute graft versus host disease (aGVHD; which occurs within the first 100 days post-transplant) is a major factor limiting the success of HCT, as is the long-term morbidity associated with chronic GVHD (cGVHD). GVHD is a risk for both HLA-matched and HLA-mismatched transplantations. GVHD can occur even if the donor and recipient are HLA-matched, because the immune system can still recognize other differences between in the donor tissues.

Both GVT and GVHD are largely mediated by conventional T cells, e.g., naïve conventional CD3⁺CD25⁻CD45RA⁺ T cells, (Tcon), which mount immune responses upon recognition of cognate antigen by T cell receptors. Depleting T cells from hematopoietic stem cell transplantation (HCT) grafts can reduce GVHD, but can also result in reduced GVT and increased likelihood of cancer relapse. Besides Tcon, Treg are an additional subset of T cells that negatively regulate inflammation and that promote immune tolerance. Treg can prevent or reduce GVHD through their negative regulation of inflammation, including, for example, inflammation elicited by donor Tcon when they recognize recipient antigens.

Provided herein are compositions and methods for improved alloHCT, comprising, for example, administering to a subject a cell population that comprise CD34+ cells (e.g., HSPC), Treg, and Tmem, and wherein the population of cells is depleted of naïve conventional αβ-T cells or naïve conventional CD3⁺CD25⁻CD45RA⁺ T cells (Tcon). Without wishing to be bound by theory, administering the cell population enriched for Treg and wherein the population of cells is depleted of naïve conventional αβ-T cells or naïve conventional CD3⁺CD25⁻CD45RA⁺ T cells (Tcon) reduces the incidence and/or severity of GVHD and/or enhances GVT. Thus, embodiments of the present disclosure provide compositions, pharmaceutical treatments, cellular therapy products, cell populations, solutions, formulations, kits, and/or methods for administering a population of immune cells, to enhance GVT while minimizing GVHD. Accordingly, the compositions, pharmaceutical treatments, cellular therapy products, cell populations, solutions, formulations, kits, and/or methods disclosed herein can retain the graft-versus-tumor (GVT) effects of alloHCT administered to a subject having a cancer (e.g., a hematologic cancer), while preventing or reducing graft versus host disease (GVHD) in the subject. In some embodiments, one or more doses of the cell populations are administered at the same time, or at different times.

### Therapeutic Agents for Hematopoietic Cell Transplantation

### Cell Populations

Embodiments of the present disclosure provide a pharmaceutical treatment or cellular therapy product to be administered to a human subject in need thereof. In some embodiments, the treatment comprises an enriched population of hematopoietic stem/progenitor cells (HSPC), memory T cells (Tmem), regulatory T cells (Treg), and invariant natural killer T cells (iNKT), wherein the population of cells is depleted of certain conventional T cells (e.g., naïve conventional αβ-T cells or naïve conventional CD3⁺CD25⁻CD45RA⁺ T cells). In some embodiments, the treatment or therapy may further include a solution comprising none or one dose of a graft vs host disease (GVHD) prophylactic agent (e.g., tacrolimus). In various embodiments, the HSPC are CD34+. In some embodiments, the cellular therapy product comprises a population of isolated CD45⁺ cells, wherein the population of isolated CD45⁺ cells comprises: a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the cellular therapy product has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH.

In certain embodiments, the present disclosure provides distinct therapeutic populations of cells that form a pharmaceutical composition or cellular therapy product useful in hematopoietic stem/progenitor cell transplant. The therapeutic population of cells can comprise an enriched population of hematopoietic stem/progenitor cells (HSPC), memory T cells (Tmem), regulatory T cells (Treg), and iNKT, and wherein the population of cells is depleted of naïve conventional αβ-T cells and/or depleted of naïve conventional CD3⁺CD25⁻CD45RA⁺ T cells (Tcon).

In some embodiments, a subject receiving the pharmaceutical treatment has received a myeloablative conditioning regimen comprising thiotepa.

In some embodiments, the distinct therapeutic population of cells comprise HSPC, Tmem and Treg is obtained from a single donor. In some embodiments, the population of cells comprise HSPC, Tmem and Treg is allogeneic relative to the human subject. In some embodiments, the population of cells comprise HSPC, Tmem and Treg is obtained from a donor that is HLA-matched relative to the human subject. In some embodiments, the population of cells comprise HSPC, Tmem and Treg is obtained from a donor that is HLA-mismatched relative to the human subject. In some embodiments, the population of cells comprise HSPC, Tmem and Treg is obtained from a donor that is haploidentical relative to the human subject. Provided herein are compositions, pharmaceutical treatments, cell populations, solutions, formulations, kits, and/or methods for improved hematopoietic stem cell transplantation (HCT), for example, allogeneic hematopoietic stem cell transplantation (alloHCT). Compositions, pharmaceutical treatments, cell populations, solutions, formulations, kits, and/or methods disclosed herein can comprise a cell population that can be administered in combination with a GVHD prophylactic agent to achieve positive clinical outcomes. Compositions, pharmaceutical treatments, cell populations, solutions, formulations, kits, and/or methods disclosed herein can comprise a cell population that can be administered with our without a GVHD prophylactic agent to achieve positive clinical outcomes. A cell population can comprise one or more types of cells, for example, hematopoietic stem and progenitor cells (HSPC), regulatory T cells (Treg), invariant natural killer T cells (iNKT), memory T cells (Tmem), and combinations thereof. A cell population can be depleted of naïve conventional αβ-T cells and/or depleted of naïve conventional CD3⁺CD25⁻CD45RA⁺ T cells (Tcon).

The disclosure provides parameters for cell populations and methods of administering cell populations that can contribute to successful clinical outcomes in HCT recipient subjects. Without wishing to be bound by theory, parameters that can contribute to successful clinical outcomes in HCT recipient subjects include, for example, conditioning regimens (e.g., myeloablative conditioning regimens), administration of a GVHD prophylactic agent as described herein (e.g., tacrolimus), doses of cells administered, timing for the administration of cells, purity standards for cells, methods for obtaining cells, methods of handling or storing cells, and combinations thereof.

In some embodiments, CD34⁺ hematopoietic stem and progenitor cells (HSPC) can have extensive self-renewal capacity, and an ability to differentiate into specialized cell types, for example, an ability to reconstitute all hematopoietic cell lineages. HSPC can undergo asynchronous replication, where two daughter cells are produced with different phenotypes. HSPC can exist in a mitotically quiescent form. HSPC can be derived from bone marrow, peripheral blood, and/or umbilical cord blood. In some embodiments, HSPC are CD34+. The HSPC can be further or alternatively described as CD133⁺, CD90+, CD38⁻, CD45RA⁻, Lin⁻, or any combination thereof. In some embodiments, the HSPC are CD19⁻, TCRα/β⁻, or a combination thereof.

In some embodiments, the HSPC are present in a composition, cellular therapy product, and/or administered to a subject in an amount or dose that may comprise from approximately 1.0×10⁵ to approximately 5.0×10⁸ HSPC per kilogram of body weight of the human subject, from approximately 1.0×10⁵ to approximately 1.0×10⁸ HSPC per kilogram of body weight of the human subject, or from approximately 5.0×10⁵ to approximately 2.0×10⁷ HSPC per kilogram of body weight of the human subject. In some embodiments, the HSPC are present in a composition, cellular therapy product, and/or administered to a subject in an amount or dose that comprises approximately 1.0×10⁵ or more HSPC per kilogram of body weight of the human subject, approximately 1.5×10⁵ or more HSPC per kilogram of body weight of the human subject, approximately 2.0×10⁵ or more HSPC per kilogram of body weight of the human subject, approximately 2.5×10⁵ or more HSPC per kilogram of body weight of the human subject, approximately 3.0×10⁵ or more HSPC per kilogram of body weight of the human subject, approximately 3.5×10⁵ or more HSPC per kilogram of body weight of the human subject, approximately 4.0×10⁵ or more HSPC per kilogram of body weight of the human subject, approximately 4.5×10⁵ or more HSPC per kilogram of body weight of the human subject, approximately 5.0×10⁵ or more HSPC per kilogram of body weight of the human subject, approximately 5.5×10⁵ or more HSPC per kilogram of body weight of the human subject, approximately 6.0×10⁵ or more HSPC per kilogram of body weight of the human subject, approximately 6.5×10⁵ or more HSPC per kilogram of body weight of the human subject, approximately 7.0×10⁵ or more HSPC per kilogram of body weight of the human subject, approximately 7.5×10⁵ or more HSPC per kilogram of body weight of the human subject, approximately 8.0×10⁵ or more HSPC per kilogram of body weight of the human subject, approximately 8.5×10⁵ or more HSPC per kilogram of body weight of the human subject, approximately 9.0×10⁵ or more HSPC per kilogram of body weight of the human subject, approximately 9.5×10⁵ or more HSPC per kilogram of body weight of the human subject, approximately 1.0×10⁶ or more HSPC per kilogram of body weight of the human subject, approximately 1.25×10⁶ or more HSPC per kilogram of body weight of the human subject, approximately 1.5×10⁶ or more HSPC per kilogram of body weight of the human subject, approximately 1.75×10⁶ or more HSPC per kilogram of body weight of the human subject, approximately 2.0×10⁶ or more HSPC per kilogram of body weight of the human subject, approximately 2.25×10⁶ or more HSPC per kilogram of body weight of the human subject, approximately 2.5×10⁶ or more HSPC per kilogram of body weight of the human subject, approximately 2.75×10⁶ or more HSPC per kilogram of body weight of the human subject, approximately 3.0×10⁶ or more HSPC per kilogram of body weight of the human subject, approximately 3.25×10⁶ or more HSPC per kilogram of body weight of the human subject, approximately 3.5×10⁶ or more HSPC per kilogram of body weight of the human subject, approximately 3.75×10⁶ or more HSPC per kilogram of body weight of the human subject, approximately 4.0×10⁶ or more HSPC per kilogram of body weight of the human subject, approximately 4.25×10⁶ or more HSPC per kilogram of body weight of the human subject, approximately 4.5×10⁶ or more HSPC per kilogram of body weight of the human subject, approximately 4.75×10⁶ or more HSPC per kilogram of body weight of the human subject, approximately 5.0×10⁶ or more HSPC per kilogram of body weight of the human subject, approximately 5.25×10⁶ or more HSPC per kilogram of body weight of the human subject, approximately 5.5×10⁶ or more HSPC per kilogram of body weight of the human subject, approximately 5.75×10⁶ or more HSPC per kilogram of body weight of the human subject, approximately 6.0×10⁶ or more HSPC per kilogram of body weight of the human subject, approximately 6.25×10⁶ or more HSPC per kilogram of body weight of the human subject, approximately 6.5×10⁶ or more HSPC per kilogram of body weight of the human subject, approximately 6.75×10⁶ or more HSPC per kilogram of body weight of the human subject, approximately 7.0×10⁶ or more HSPC per kilogram of body weight of the human subject, approximately 7.25×10⁶ or more HSPC per kilogram of body weight of the human subject, approximately 7.5×10⁶ or more HSPC per kilogram of body weight of the human subject, approximately 7.75×10⁶ or more HSPC per kilogram of body weight of the human subject, approximately 8.0×10⁶ or more HSPC per kilogram of body weight of the human subject, approximately 8.25×10⁶ or more HSPC per kilogram of body weight of the human subject, approximately 8.5×10⁶ or more HSPC per kilogram of body weight of the human subject, approximately 8.75×10⁶ or more HSPC per kilogram of body weight of the human subject, approximately 9.0×10⁶ or more HSPC per kilogram of body weight of the human subject, approximately 9.25×10⁶ or more HSPC per kilogram of body weight of the human subject, approximately 9.5×10⁶ or more HSPC per kilogram of body weight of the human subject, approximately 9.75×10⁶ or more HSPC per kilogram of body weight of the human subject, approximately 1.0×10⁷ or more HSPC per kilogram of body weight of the human subject, approximately 1.25×10⁷ or more HSPC per kilogram of body weight of the human subject, approximately 1.5×10⁷ or more HSPC per kilogram of body weight of the human subject, approximately 1.75×10⁷ or more HSPC per kilogram of body weight of the human subject, approximately 2.0×10⁷ or more HSPC per kilogram of body weight of the human subject, approximately 2.25×10⁷ or more HSPC per kilogram of body weight of the human subject, approximately 2.5×10⁷ or more HSPC per kilogram of body weight of the human subject, approximately 2.75×10⁷ or more HSPC per kilogram of body weight of the human subject, approximately 3.0×10⁷ or more HSPC per kilogram of body weight of the human subject, approximately 3.25×10⁷ or more HSPC per kilogram of body weight of the human subject, approximately 3.5×10⁷ or more HSPC per kilogram of body weight of the human subject, approximately 3.75×10⁷ or more HSPC per kilogram of body weight of the human subject, approximately 4.0×10⁷ or more HSPC per kilogram of body weight of the human subject, approximately 4.25×10⁷ or more HSPC per kilogram of body weight of the human subject, approximately 4.5×10⁷ or more HSPC per kilogram of body weight of the human subject, approximately 4.75×10⁷ or more HSPC per kilogram of body weight of the human subject, approximately 5.0×10⁷ or more HSPC per kilogram of body weight of the human subject, approximately 5.25×10⁷ or more HSPC per kilogram of body weight of the human subject, approximately 5.5×10⁷ or more HSPC per kilogram of body weight of the human subject, approximately 5.75×10⁷ or more HSPC per kilogram of body weight of the human subject, approximately 6.0×10⁷ or more HSPC per kilogram of body weight of the human subject, approximately 6.25×10⁷ or more HSPC per kilogram of body weight of the human subject, approximately 6.5×10⁷ or more HSPC per kilogram of body weight of the human subject, approximately 6.75×10⁷ or more HSPC per kilogram of body weight of the human subject, approximately 7.0×10⁷ or more HSPC per kilogram of body weight of the human subject, approximately 7.25×10⁷ or more HSPC per kilogram of body weight of the human subject, approximately 7.5×10⁷ or more HSPC per kilogram of body weight of the human subject, approximately 7.75×10⁷ or more HSPC per kilogram of body weight of the human subject, approximately 8.0×10⁷ or more HSPC per kilogram of body weight of the human subject, approximately 8.25×10⁷ or more HSPC per kilogram of body weight of the human subject, approximately 8.5×10⁷ or more HSPC per kilogram of body weight of the human subject, approximately 8.75×10⁷ or more HSPC per kilogram of body weight of the human subject, approximately 9.0×10⁷ or more HSPC per kilogram of body weight of the human subject, approximately 9.25×10⁷ or more HSPC per kilogram of body weight of the human subject, approximately 9.5×10⁷ or more HSPC per kilogram of body weight of the human subject, approximately 9.75×10⁷ or more HSPC per kilogram of body weight of the human subject, approximately 1.0×10⁸ or more HSPC per kilogram of body weight of the human subject, approximately 1.25×10⁸ or more HSPC per kilogram of body weight of the human subject, approximately 1.5×10⁸ or more HSPC per kilogram of body weight of the human subject, approximately 1.75×10⁸ or more HSPC per kilogram of body weight of the human subject, approximately 2.0×10⁸ or more HSPC per kilogram of body weight of the human subject, approximately 2.25×10⁸ or more HSPC per kilogram of body weight of the human subject, approximately 2.5×10⁸ or more HSPC per kilogram of body weight of the human subject, approximately 2.75×10⁸ or more HSPC per kilogram of body weight of the human subject, approximately 3.0×10⁸ or more HSPC per kilogram of body weight of the human subject, approximately 3.25×10⁸ or more HSPC per kilogram of body weight of the human subject, approximately 3.5×10⁸ or more HSPC per kilogram of body weight of the human subject, approximately 3.75×10⁸ or more HSPC per kilogram of body weight of the human subject, approximately 4.0×10⁸ or more HSPC per kilogram of body weight of the human subject, approximately 4.25×10⁸ or more HSPC per kilogram of body weight of the human subject, approximately 4.5×10⁸ or more HSPC per kilogram of body weight of the human subject, approximately 4.75×10⁸ or more HSPC per kilogram of body weight of the human subject, or approximately 5.0×10⁸ or more HSPC per kilogram of body weight of the human subject. In some embodiments, the body weight of the human subject is actual body weight of the human subject. In other embodiments, the body weight of the human subject is ideal actual body weight of the human subject.

Subsets of immune cells, such as naïve conventional αβ-T cells or naïve conventional CD3⁺CD25⁻CD45RA⁺ T cells (Tcon), regulatory T cells (Treg), invariant natural killer T cells (iNKT), and memory T cells (Tmem) can contribute to aspects of GVHD following HCT, and can also contribute to, for example, GVT immune responses, immune reconstitution, infection susceptibility, and patient survival.

Regulatory T cells (“Treg”) are a specialized subpopulation of T cells that negatively regulate (e.g., suppress) activation of the immune system and thereby promote immune tolerance. There are a number of subsets of Treg, for example, TCRαβ+CD4+ regulatory T cells, which include natural regulatory T cells (nTreg) and induced regulatory T cells (iTreg). nTreg and iTreg are CD4⁺CD25+; both can inhibit proliferation of CD4⁺CD25− T cells in a dose-dependent manner. In some embodiments, Treg are anergic and do not proliferate upon TCR stimulation. In addition to being positive for CD4 and CD25, Treg can be positive for the transcription factor FOXP3, an intracellular marker. Treg can be identified or selected based on various marker expression profiles. Non-limiting examples of marker expression profiles that can be used to select Treg include (1) CD4+CD25+CD127dim, (2) CD4+FOXP3+, (3) CD3+CD4+CD25+, (5) CD3+CD4+CD25+CD127dim, (6) CD3+CD4+CD25+CD127dim FOXP3+, (7) CD3+FOXP3+, (8) CD3+CD4+FOXP3+, (9) CD3+CD4+CD25+FOXP3+, (10) CD3+CD25+FOXP3+, (11) CD3+CD25+CD127dim, (12) CD4+CD25+, (13) CD4+CD25+CD127dimFOXP3+, (14) FOXP3+, CD4+FOXP3+, (15) CD4+CD25+FOXP3+, (16) CD25+FOXP3+, and (17) CD25+CD127dim. Selection based on certain expression profiles can be achieved based on extracellular markers and without requiring cell permeabilization, for example, selection based on CD4⁺CD25⁺CD127dim.

A cell population that comprises Treg can, for example, reduce the incidence of graft rejection, reduce the incidence and/or severity of GVHD, promote hematopoietic reconstitution, promote immune reconstitution, promote mixed chimerism, or a combination thereof.

In some embodiments, the Treg are CD4⁺CD25⁺CD127dim or CD4+FOXP3+. In some embodiments, the Treg are CD4⁺CD25⁺CD127^{dim} and are also FOXP3+. In some embodiments Treg of the present disclosure are fresh Treg. In some embodiments, Treg of the present disclosure are fresh CD4⁺CD25⁺CD127^{dim} regulatory T cells (Treg).

In some embodiments, Treg are present in a composition, cellular therapy product, and/or administered to a subject in an amount or dose that may comprise from approximately 1.0×10⁵ to approximately 1.0×10⁸ Treg per kilogram of body weight of the human subject, from approximately 1.0×10⁵ to approximately 2.0×10⁷ Treg per kilogram of body weight of the human subject, from approximately 1.0×10⁵ to approximately 1.0×10⁷ Treg per kilogram of body weight of the human subject, or from approximately 5.0×10⁵ to approximately 4.0×10⁶ Treg per kilogram of body weight of the human subject. In some embodiments, the Treg are present in a composition, cellular therapy product, and/or administered to a subject in an amount or dose that comprises approximately 1.0×10⁵ or more Treg per kilogram of body weight of the human subject, approximately 1.5×10⁵ or more Treg per kilogram of body weight of the human subject, approximately 2.0×10⁵ or more Treg per kilogram of body weight of the human subject, approximately 2.5×10⁵ or more Treg per kilogram of body weight of the human subject, approximately 3.0×10⁵ or more Treg per kilogram of body weight of the human subject, approximately 3.5×10⁵ or more Treg per kilogram of body weight of the human subject, approximately 4.0×10⁵ or more Treg per kilogram of body weight of the human subject, approximately 4.5×10⁵ or more Treg per kilogram of body weight of the human subject, approximately 5.0×10⁵ or more Treg per kilogram of body weight of the human subject, approximately 5.5×10⁵ or more Treg per kilogram of body weight of the human subject, approximately 6.0×10⁵ or more Treg per kilogram of body weight of the human subject, approximately 6.5×10⁵ or more Treg per kilogram of body weight of the human subject, approximately 7.0×10⁵ or more Treg per kilogram of body weight of the human subject, approximately 7.5×10⁵ or more Treg per kilogram of body weight of the human subject, approximately 8.0×10⁵ or more Treg per kilogram of body weight of the human subject, approximately 8.5×10⁵ or more Treg per kilogram of body weight of the human subject, approximately 9.0×10⁵ or more Treg per kilogram of body weight of the human subject, approximately 9.5×10⁵ or more Treg per kilogram of body weight of the human subject, approximately 1.0×10⁶ or more Treg per kilogram of body weight of the human subject, approximately 1.25×10⁶ or more Treg per kilogram of body weight of the human subject, approximately 1.5×10⁶ or more Treg per kilogram of body weight of the human subject, approximately 1.75×10⁶ or more Treg per kilogram of body weight of the human subject, approximately 2.0×10⁶ or more Treg per kilogram of body weight of the human subject, approximately 2.25×10⁶ or more Treg per kilogram of body weight of the human subject, approximately 2.5×10⁶ or more Treg per kilogram of body weight of the human subject, approximately 2.75×10⁶ or more Treg per kilogram of body weight of the human subject, approximately 3.0×10⁶ or more Treg per kilogram of body weight of the human subject, approximately 3.25×10⁶ or more Treg per kilogram of body weight of the human subject, approximately 3.5×10⁶ or more Treg per kilogram of body weight of the human subject, approximately 3.75×10⁶ or more Treg per kilogram of body weight of the human subject, approximately 4.0×10⁶ or more Treg per kilogram of body weight of the human subject, approximately 4.25×10⁶ or more Treg per kilogram of body weight of the human subject, approximately 4.5×10⁶ or more Treg per kilogram of body weight of the human subject, approximately 4.75×10⁶ or more Treg per kilogram of body weight of the human subject, approximately 5.0×10⁶ or more Treg per kilogram of body weight of the human subject, approximately 5.25×10⁶ or more Treg per kilogram of body weight of the human subject, approximately 5.5×10⁶ or more Treg per kilogram of body weight of the human subject, approximately 5.75×10⁶ or more Treg per kilogram of body weight of the human subject, approximately 6.0×10⁶ or more Treg per kilogram of body weight of the human subject, approximately 6.25×10⁶ or more Treg per kilogram of body weight of the human subject, approximately 6.5×10⁶ or more Treg per kilogram of body weight of the human subject, approximately 6.75×10⁶ or more Treg per kilogram of body weight of the human subject, approximately 7.0×10⁶ or more Treg per kilogram of body weight of the human subject, approximately 7.25×10⁶ or more Treg per kilogram of body weight of the human subject, approximately 7.5×10⁶ or more Treg per kilogram of body weight of the human subject, approximately 7.75×10⁶ or more Treg per kilogram of body weight of the human subject, approximately 8.0×10⁶ or more Treg per kilogram of body weight of the human subject, approximately 8.25×10⁶ or more Treg per kilogram of body weight of the human subject, approximately 8.5×10⁶ or more Treg per kilogram of body weight of the human subject, approximately 8.75×10⁶ or more Treg per kilogram of body weight of the human subject, approximately 9.0×10⁶ or more Treg per kilogram of body weight of the human subject, approximately 9.25×10⁶ or more Treg per kilogram of body weight of the human subject, approximately 9.5×10⁶ or more Treg per kilogram of body weight of the human subject, approximately 9.75×10⁶ or more Treg per kilogram of body weight of the human subject, approximately 1.0×10⁷ or more Treg per kilogram of body weight of the human subject, approximately 1.25×10⁷ or more Treg per kilogram of body weight of the human subject, approximately 1.5×10⁷ or more Treg per kilogram of body weight of the human subject, approximately 1.75×10⁷ or more Treg per kilogram of body weight of the human subject, approximately 2.0×10⁷ or more Treg per kilogram of body weight of the human subject, approximately 2.25×10⁷ or more Treg per kilogram of body weight of the human subject, approximately 2.5×10⁷ or more Treg per kilogram of body weight of the human subject, approximately 2.75×10⁷ or more Treg per kilogram of body weight of the human subject, approximately 3.0×10⁷ or more Treg per kilogram of body weight of the human subject, approximately 3.25×10⁷ or more Treg per kilogram of body weight of the human subject, approximately 3.5×10⁷ or more Treg per kilogram of body weight of the human subject, approximately 3.75×10⁷ or more Treg per kilogram of body weight of the human subject, approximately 4.0×10⁷ or more Treg per kilogram of body weight of the human subject, approximately 4.25×10⁷ or more Treg per kilogram of body weight of the human subject, approximately 4.5×10⁷ or more Treg per kilogram of body weight of the human subject, approximately 4.75×10⁷ or more Treg per kilogram of body weight of the human subject, approximately 5.0×10⁷ or more Treg per kilogram of body weight of the human subject, approximately 5.25×10⁷ or more Treg per kilogram of body weight of the human subject, approximately 5.5×10⁷ or more Treg per kilogram of body weight of the human subject, approximately 5.75×10⁷ or more Treg per kilogram of body weight of the human subject, approximately 6.0×10⁷ or more Treg per kilogram of body weight of the human subject, approximately 6.25×10⁷ or more Treg per kilogram of body weight of the human subject, approximately 6.5×10⁷ or more Treg per kilogram of body weight of the human subject, approximately 6.75×10⁷ or more Treg per kilogram of body weight of the human subject, approximately 7.0×10⁷ or more Treg per kilogram of body weight of the human subject, approximately 7.25×10⁷ or more Treg per kilogram of body weight of the human subject, approximately 7.5×10⁷ or more Treg per kilogram of body weight of the human subject, approximately 7.75×10⁷ or more Treg per kilogram of body weight of the human subject, approximately 8.0×10⁷ or more Treg per kilogram of body weight of the human subject, approximately 8.25×10⁷ or more Treg per kilogram of body weight of the human subject, approximately 8.5×10⁷ or more Treg per kilogram of body weight of the human subject, approximately 8.75×10⁷ or more Treg per kilogram of body weight of the human subject, approximately 9.0×10⁷ or more Treg per kilogram of body weight of the human subject, approximately 9.25×10⁷ or more Treg per kilogram of body weight of the human subject, approximately 9.5×10⁷ or more Treg per kilogram of body weight of the human subject, approximately 9.75×10⁷ or more Treg per kilogram of body weight of the human subject, approximately 1.0×10⁸ or more Treg per kilogram of body weight of the human subject. In some embodiments, the body weight of the human subject is actual body weight of the human subject. In other embodiments, the body weight of the human subject is ideal actual body weight of the human subject.

A cell population of the disclosure can comprise memory T cells (Tmem). Tmem can refer to antigen-experienced T cells that express, for example, any of the phenotypic markers CD45RO, TCRα, TCRβ, CD3, CD4, CD95, and IL-2Rβ or the phenotypic markers CD45RO, TCRα, TCRβ, CD3, CD8, CD95, and IL-2Rβ. Tmem provide immunity and are capable of persisting for a long period of time in an inactive state. Tmem are able to rapidly acquire effector functions upon re-challenge with antigen. A population of Tmem can include any combination of the subclasses T central memory cells and T effector memory cells. In some embodiments, Tmem of the present disclosure are CD45RA−. In some embodiments, Tmem of the present disclosure are CD3⁺CD45RA-CD45RO⁺. In various methods, Tmem administered to a subject receiving HCT can, for example, promote GVT, reduce GVHD, decrease susceptibility to cancer relapse, decrease susceptibility to infection, or a combination thereof.

In some embodiments, the Tmem are present in a composition, cellular therapy product, and/or administered to a subject in an amount or dose that may comprise from approximately 1.0×10⁵ to approximately 5.0×10⁸ Tmem per kilogram of body weight of the human subject, from approximately 1.0×10⁵ to approximately 1.0×10⁸ Tmem per kilogram of body weight of the human subject, or from approximately 5.0×10⁵ to approximately 2.0×10⁷ Tmem per kilogram of body weight of the human subject. In some embodiments, the Tmem are present in a composition, cellular therapy product, and/or administered to a subject in an amount or dose that comprises approximately 1.0×10⁵ or more Tmem per kilogram of body weight of the human subject, approximately 1.5×10⁵ or more Tmem per kilogram of body weight of the human subject, approximately 2.0×10⁵ or more Tmem per kilogram of body weight of the human subject, approximately 2.5×10⁵ or more Tmem per kilogram of body weight of the human subject, approximately 3.0×10⁵ or more Tmem per kilogram of body weight of the human subject, approximately 3.5×10⁵ or more Tmem per kilogram of body weight of the human subject, approximately 4.0×10⁵ or more Tmem per kilogram of body weight of the human subject, approximately 4.5×10⁵ or more Tmem per kilogram of body weight of the human subject, approximately 5.0×10⁵ or more Tmem per kilogram of body weight of the human subject, approximately 5.5×10⁵ or more Tmem per kilogram of body weight of the human subject, approximately 6.0×10⁵ or more Tmem per kilogram of body weight of the human subject, approximately 6.5×10⁵ or more Tmem per kilogram of body weight of the human subject, approximately 7.0×10⁵ or more Tmem per kilogram of body weight of the human subject, approximately 7.5×10⁵ or more Tmem per kilogram of body weight of the human subject, approximately 8.0×10⁵ or more Tmem per kilogram of body weight of the human subject, approximately 8.5×10⁵ or more Tmem per kilogram of body weight of the human subject, approximately 9.0×10⁵ or more Tmem per kilogram of body weight of the human subject, approximately 9.5×10⁵ or more Tmem per kilogram of body weight of the human subject, approximately 1.0×10⁶ or more Tmem per kilogram of body weight of the human subject, approximately 1.25×10⁶ or more Tmem per kilogram of body weight of the human subject, approximately 1.5×10⁶ or more Tmem per kilogram of body weight of the human subject, approximately 1.75×10⁶ or more Tmem per kilogram of body weight of the human subject, approximately 2.0×10⁶ or more Tmem per kilogram of body weight of the human subject, approximately 2.25×10⁶ or more Tmem per kilogram of body weight of the human subject, approximately 2.5×10⁶ or more Tmem per kilogram of body weight of the human subject, approximately 2.75×10⁶ or more Tmem per kilogram of body weight of the human subject, approximately 3.0×10⁶ or more Tmem per kilogram of body weight of the human subject, approximately 3.25×10⁶ or more Tmem per kilogram of body weight of the human subject, approximately 3.5×10⁶ or more Tmem per kilogram of body weight of the human subject, approximately 3.75×10⁶ or more Tmem per kilogram of body weight of the human subject, approximately 4.0×10⁶ or more Tmem per kilogram of body weight of the human subject, approximately 4.25×10⁶ or more Tmem per kilogram of body weight of the human subject, approximately 4.5×10⁶ or more Tmem per kilogram of body weight of the human subject, approximately 4.75×10⁶ or more Tmem per kilogram of body weight of the human subject, approximately 5.0×10⁶ or more Tmem per kilogram of body weight of the human subject, approximately 5.25×10⁶ or more Tmem per kilogram of body weight of the human subject, approximately 5.5×10⁶ or more Tmem per kilogram of body weight of the human subject, approximately 5.75×10⁶ or more Tmem per kilogram of body weight of the human subject, approximately 6.0×10⁶ or more Tmem per kilogram of body weight of the human subject, approximately 6.25×10⁶ or more Tmem per kilogram of body weight of the human subject, approximately 6.5×10⁶ or more Tmem per kilogram of body weight of the human subject, approximately 6.75×10⁶ or more Tmem per kilogram of body weight of the human subject, approximately 7.0×10⁶ or more Tmem per kilogram of body weight of the human subject, approximately 7.25×10⁶ or more Tmem per kilogram of body weight of the human subject, approximately 7.5×10⁶ or more Tmem per kilogram of body weight of the human subject, approximately 7.75×10⁶ or more Tmem per kilogram of body weight of the human subject, approximately 8.0×10⁶ or more Tmem per kilogram of body weight of the human subject, approximately 8.25×10⁶ or more Tmem per kilogram of body weight of the human subject, approximately 8.5×10⁶ or more Tmem per kilogram of body weight of the human subject, approximately 8.75×10⁶ or more Tmem per kilogram of body weight of the human subject, approximately 9.0×10⁶ or more Tmem per kilogram of body weight of the human subject, approximately 9.25×10⁶ or more Tmem per kilogram of body weight of the human subject, approximately 9.5×10⁶ or more Tmem per kilogram of body weight of the human subject, approximately 9.75×10⁶ or more Tmem per kilogram of body weight of the human subject, approximately 1.0×10⁷ or more Tmem per kilogram of body weight of the human subject, approximately 1.25×10⁷ or more Tmem per kilogram of body weight of the human subject, approximately 1.5×10⁷ or more Tmem per kilogram of body weight of the human subject, approximately 1.75×10⁷ or more Tmem per kilogram of body weight of the human subject, approximately 2.0×10⁷ or more Tmem per kilogram of body weight of the human subject, approximately 2.25×10⁷ or more Tmem per kilogram of body weight of the human subject, approximately 2.5×10⁷ or more Tmem per kilogram of body weight of the human subject, approximately 2.75×10⁷ or more Tmem per kilogram of body weight of the human subject, approximately 3.0×10⁷ or more Tmem per kilogram of body weight of the human subject, approximately 3.25×10⁷ or more Tmem per kilogram of body weight of the human subject, approximately 3.5×10⁷ or more Tmem per kilogram of body weight of the human subject, approximately 3.75×10⁷ or more Tmem per kilogram of body weight of the human subject, approximately 4.0×10⁷ or more Tmem per kilogram of body weight of the human subject, approximately 4.25×10⁷ or more Tmem per kilogram of body weight of the human subject, approximately 4.5×10⁷ or more Tmem per kilogram of body weight of the human subject, approximately 4.75×10⁷ or more Tmem per kilogram of body weight of the human subject, approximately 5.0×10⁷ or more Tmem per kilogram of body weight of the human subject, approximately 5.25×10⁷ or more Tmem per kilogram of body weight of the human subject, approximately 5.5×10⁷ or more Tmem per kilogram of body weight of the human subject, approximately 5.75×10⁷ or more Tmem per kilogram of body weight of the human subject, approximately 6.0×10⁷ or more Tmem per kilogram of body weight of the human subject, approximately 6.25×10⁷ or more Tmem per kilogram of body weight of the human subject, approximately 6.5×10⁷ or more Tmem per kilogram of body weight of the human subject, approximately 6.75×10⁷ or more Tmem per kilogram of body weight of the human subject, approximately 7.0×10⁷ or more Tmem per kilogram of body weight of the human subject, approximately 7.25×10⁷ or more Tmem per kilogram of body weight of the human subject, approximately 7.5×10⁷ or more Tmem per kilogram of body weight of the human subject, approximately 7.75×10⁷ or more Tmem per kilogram of body weight of the human subject, approximately 8.0×10⁷ or more Tmem per kilogram of body weight of the human subject, approximately 8.25×10⁷ or more Tmem per kilogram of body weight of the human subject, approximately 8.5×10⁷ or more Tmem per kilogram of body weight of the human subject, approximately 8.75×10⁷ or more Tmem per kilogram of body weight of the human subject, approximately 9.0×10⁷ or more Tmem per kilogram of body weight of the human subject, approximately 9.25×10⁷ or more Tmem per kilogram of body weight of the human subject, approximately 9.5×10⁷ or more Tmem per kilogram of body weight of the human subject, approximately 9.75×10⁷ or more Tmem per kilogram of body weight of the human subject, approximately 1.0×10⁸ or more Tmem per kilogram of body weight of the human subject, approximately 1.25×10⁸ or more Tmem per kilogram of body weight of the human subject, approximately 1.5×10⁸ or more Tmem per kilogram of body weight of the human subject, approximately 1.75×10⁸ or more Tmem per kilogram of body weight of the human subject, approximately 2.0×10⁸ or more Tmem per kilogram of body weight of the human subject, approximately 2.25×10⁸ or more Tmem per kilogram of body weight of the human subject, approximately 2.5×10⁸ or more Tmem per kilogram of body weight of the human subject, approximately 2.75×10⁸ or more Tmem per kilogram of body weight of the human subject, approximately 3.0×10⁸ or more Tmem per kilogram of body weight of the human subject, approximately 3.25×10⁸ or more Tmem per kilogram of body weight of the human subject, approximately 3.5×10⁸ or more Tmem per kilogram of body weight of the human subject, approximately 3.75×10⁸ or more Tmem per kilogram of body weight of the human subject, approximately 4.0×10⁸ or more Tmem per kilogram of body weight of the human subject, approximately 4.25×10⁸ or more Tmem per kilogram of body weight of the human subject, approximately 4.5×10⁸ or more Tmem per kilogram of body weight of the human subject, approximately 4.75×10⁸ or more Tmem per kilogram of body weight of the human subject, or approximately 5.0×10⁸ or more Tmem per kilogram of body weight of the human subject. In some embodiments, the body weight of the human subject is actual body weight of the human subject. In other embodiments, the body weight of the human subject is ideal actual body weight of the human subject.

A cell population of the disclosure can comprise invariant natural killer T cells (iNKT). iNKT are subclass of CD1d-restricted Natural Killer T (NKT) cells that express a highly conserved αβ-T cell receptor that comprises of Vα24Jα18 TCRα chain in humans (referred to herein as “Vα24Jα18+”). iNKT cells can be identified by binding with CD1d-multimers like that are loaded with α-galactosylceramide (GalCer), PBS-57, PBS-44 or other natural or synthetic glycolipids. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells. iNKT can be CD3+Vα24Jα18+. In some embodiments, iNKT of the present disclosure are TCR Vα24Jα18⁺CD127⁺.

In some embodiments, iNKT can promote engraftment, promote GVT, reduce incidence and/or severity of GVHD, decrease susceptibility to cancer relapse, decrease susceptibility to infection, or a combination thereof. In some embodiments, iNKT promote the activity of Treg. In some embodiments, iNKT promote the activity of HSPC.

In some embodiments, the iNKT are present in a composition, cellular therapy product, and/or administered to a subject in an amount or dose that may comprise from about 1×10⁴ to about 1×10⁹ iNKT per kilogram of actual body weight or ideal body weight of the human subject. In some embodiments, the iNKT are present in a composition, cellular therapy product, and/or administered to a subject in an amount or dose that may comprise from approximately 5.0×10² to approximately 2.0×10⁶ iNKT per kilogram of body weight of the human subject. In some embodiments, the iNKT are present in a composition, cellular therapy product, and/or administered to a subject in an amount or dose that comprises approximately 5.0×10² or more iNKT per kilogram of body weight of the human subject, approximately 6.0×10² or more iNKT per kilogram of body weight of the human subject, approximately 7.0×10² or more iNKT per kilogram of body weight of the human subject, approximately 8.0×10² or more iNKT per kilogram of body weight of the human subject, approximately 9.0×10² or more iNKT per kilogram of body weight of the human subject, approximately 1.0×10³ or more iNKT per kilogram of body weight of the human subject, approximately 2.0×10³ or more iNKT per kilogram of body weight of the human subject, approximately 3.0×10³ or more iNKT per kilogram of body weight of the human subject, approximately 4.0×10³ or more iNKT per kilogram of body weight of the human subject, approximately 5.0×10³ or more iNKT per kilogram of body weight of the human subject, approximately 6.0×10³ or more iNKT per kilogram of body weight of the human subject, approximately 7.0×10³ or more iNKT per kilogram of body weight of the human subject, approximately 8.0×10³ or more iNKT per kilogram of body weight of the human subject, approximately 9.0×10³ or more iNKT per kilogram of body weight of the human subject, approximately 1.0×10⁴ or more iNKT per kilogram of body weight of the human subject, approximately 2.0×10⁴ or more iNKT per kilogram of body weight of the human subject, approximately 3.0×10⁴ or more iNKT per kilogram of body weight of the human subject, approximately 4.0×10⁴ or more iNKT per kilogram of body weight of the human subject, approximately 5.0×10⁴ or more iNKT per kilogram of body weight of the human subject, approximately 6.0×10⁴ or more iNKT per kilogram of body weight of the human subject, approximately 7.0×10⁴ or more iNKT per kilogram of body weight of the human subject, approximately 8.0×10⁴ or more iNKT per kilogram of body weight of the human subject, approximately 9.0×10⁴ or more iNKT per kilogram of body weight of the human subject, approximately 1.0×10⁵ or more iNKT per kilogram of body weight of the human subject, approximately 2.0×10⁵ or more iNKT per kilogram of body weight of the human subject, approximately 3.0×10⁵ or more iNKT per kilogram of body weight of the human subject, approximately 4.0×10⁵ or more iNKT per kilogram of body weight of the human subject, approximately 5.0×10⁵ or more iNKT per kilogram of body weight of the human subject, approximately 6.0×10⁵ or more iNKT per kilogram of body weight of the human subject, approximately 7.0×10⁵ or more iNKT per kilogram of body weight of the human subject, approximately 8.0×10⁵ or more iNKT per kilogram of body weight of the human subject, approximately 9.0×10⁵ or more iNKT per kilogram of body weight of the human subject, approximately 1.0×10⁶ or more iNKT per kilogram of body weight of the human subject, approximately 1.5×10⁶ or more iNKT per kilogram of body weight of the human subject, or approximately 2.0×10⁶ or more iNKT per kilogram of body weight of the human subject.

As used herein, the term “naïve conventional αβ-T cells” or “naïve conventional CD3⁺CD25⁻CD45RA⁺ T cells” refers to one ore more non-antigen experienced T cell that express the phenotypic markers TCRα/(3, CD45RA, and express medium to high levels of CD127 (CD127+), and do not express or have low expression of CD45RO and CD25. In some embodiments, naïve conventional αβ-T cells or naïve conventional CD3⁺CD25⁻CD45RA⁺ T cells (Tcon) are characterized by the expression of phenotypic markers of naïve conventional αβ-T cells including TCRα, TCRβ, CD3, CD4 or CD8, CD62L, CCR7, CD28, CD127, and CD45RA. In some embodiments naïve conventional αβ-T cells or naïve conventional CD3⁺CD25⁻CD45RA⁺ T cells (Tcon) are CD3⁺CD25⁻ CD45RA⁺ and comprise a polymorphic TCRα, and/or a polymorphic TCRβ. In some embodiments, naïve conventional αβ-T cells or naïve conventional CD3⁺CD25⁻CD45RA⁺ T cells (Tcon) do not express the Vα24Jα18 TCR found on iNKT cells. In some embodiments, the naïve conventional αβ-T cells or naïve conventional CD3⁺CD25⁻CD45RA⁺ T cells (Tcon) are CD45RA⁺ and are sorted from a cell sample which has already been sorted for HSPC, Treg, and Tmem cells.

In some embodiments, the naïve conventional αβ-T cells or naïve conventional CD3⁺CD25⁻CD45RA⁺ T cells (Tcon) are CD25⁻, CD45RA⁺ or any combination thereof. In some embodiments, the naïve conventional αβ-T cells or naïve conventional CD3⁺CD25⁻CD45RA⁺ T cells (Tcon) can be TCRα/β⁺ CD45RA⁺ and CD25⁻, CD127+, or both. The naïve conventional αβ-T cells or naïve conventional CD3⁺CD25⁻CD45RA⁺ T cells (Tcon) can be further described as TCRα+TCRβ⁺ CD45RA⁺ CD45RO⁻ CD25⁻CD95⁻ IL-2β⁻ CD127⁺ Vα24Jα18⁻. The CD45RA⁺ naïve conventional αβ-T cells or Tcon may be discarded during or after cell sorting, for the depletion of naïve conventional αβ-T cells or depletion of naïve conventional CD3⁺CD25⁻CD45RA⁺ T cells (Tcon).

In some embodiments, the naïve conventional αβ-T cells or naïve conventional CD3⁺CD25⁻CD45RA⁺ T cells (Tcon) are present in a composition, cellular therapy product, and/or administered to a subject in an amount that is less than approximately 5.0×10⁵ naïve conventional CD3⁺CD25⁻CD45RA⁺ T cells (Tcon) per kilogram of body weight of the human subject. In some embodiments, the naïve conventional αβ-T cells or naïve conventional CD3⁺CD25⁻CD45RA⁺ T cells (Tcon) or are present in a composition, cellular therapy product, and/or administered to a subject in an amount that is less than approximately 1.0×10⁵ naïve conventional CD3⁺CD25⁻CD45RA⁺ T cells (Tcon) per kilogram of body weight of the human subject. In some embodiments, the Tcon are present in a composition, cellular therapy product, and/or administered to a subject in an amount that is less than approximately 5.0×10⁵ Tcon per kilogram of body weight of the human subject, less than approximately 4.9×10⁵ Tcon per kilogram of body weight of the human subject, less than approximately 4.8×10⁵ Tcon per kilogram of body weight of the human subject, less than approximately 4.7×10⁵ Tcon per kilogram of body weight of the human subject, less than approximately 4.6×10⁵ Tcon per kilogram of body weight of the human subject, less than approximately 4.5×10⁵ Tcon per kilogram of body weight of the human subject, less than approximately 4.4×10⁵ Tcon per kilogram of body weight of the human subject, less than approximately 4.3×10⁵ Tcon per kilogram of body weight of the human subject, less than approximately 4.2×10⁵ Tcon per kilogram of body weight of the human subject, less than approximately 4.1×10⁵ Tcon per kilogram of body weight of the human subject, less than approximately 4.0×10⁵ Tcon per kilogram of body weight of the human subject, less than approximately 3.9×10⁵ Tcon per kilogram of body weight of the human subject, less than approximately 3.8×10⁵ Tcon per kilogram of body weight of the human subject, less than approximately 3.7×10⁵ Tcon per kilogram of body weight of the human subject, less than approximately 3.6×10⁵ Tcon per kilogram of body weight of the human subject, less than approximately 3.5×10⁵ Tcon per kilogram of body weight of the human subject, less than approximately 3.4×10⁵ Tcon per kilogram of body weight of the human subject, less than approximately 3.3×10⁵ Tcon per kilogram of body weight of the human subject, less than approximately 3.2×10⁵ Tcon per kilogram of body weight of the human subject, less than approximately 3.1×10⁵ Tcon per kilogram of body weight of the human subject, less than approximately 3.0×10⁵ Tcon per kilogram of body weight of the human subject, less than approximately 2.9×10⁵ Tcon per kilogram of body weight of the human subject, less than approximately 2.8×10⁵ Tcon per kilogram of body weight of the human subject, less than approximately 2.7×10⁵ Tcon per kilogram of body weight of the human subject, less than approximately 2.6×10⁵ Tcon per kilogram of body weight of the human subject, less than approximately 2.5×10⁵ Tcon per kilogram of body weight of the human subject, less than approximately 2.4×10⁵ Tcon per kilogram of body weight of the human subject, less than approximately 2.3×10⁵ Tcon per kilogram of body weight of the human subject, less than approximately 2.2×10⁵ Tcon per kilogram of body weight of the human subject, less than approximately 2.1×10⁵ Tcon per kilogram of body weight of the human subject, less than approximately 2.0×10⁵ Tcon per kilogram of body weight of the human subject, less than approximately 1.9×10⁵ Tcon per kilogram of body weight of the human subject, less than approximately 1.8×10⁵ Tcon per kilogram of body weight of the human subject, less than approximately 1.7×10⁵ Tcon per kilogram of body weight of the human subject, less than approximately 1.6×10⁵ Tcon per kilogram of body weight of the human subject, less than approximately 1.5×10⁵ Tcon per kilogram of body weight of the human subject, less than approximately 1.4×10⁵ Tcon per kilogram of body weight of the human subject, less than approximately 1.3×10⁵ Tcon per kilogram of body weight of the human subject, less than approximately 1.2×10⁵ Tcon per kilogram of body weight of the human subject, less than approximately 1.1×10⁵ Tcon per kilogram of body weight of the human subject, less than approximately 1.0×10⁵ Tcon per kilogram of body weight of the human subject, less than approximately 9.9×10⁴ Tcon per kilogram of body weight of the human subject, less than approximately 9.8×10⁴ Tcon per kilogram of body weight of the human subject, less than approximately 9.7×10⁴ Tcon per kilogram of body weight of the human subject, less than approximately 9.6×10⁴ Tcon per kilogram of body weight of the human subject, less than approximately 9.5×10⁴ Tcon per kilogram of body weight of the human subject, less than approximately 9.4×10⁴ Tcon per kilogram of body weight of the human subject, less than approximately 9.3×10⁴ Tcon per kilogram of body weight of the human subject, less than approximately 9.2×10⁴ Tcon per kilogram of body weight of the human subject, less than approximately 9.1×10⁴ Tcon per kilogram of body weight of the human subject, less than approximately 9.0×10⁴ Tcon per kilogram of body weight of the human subject, less than approximately 8.9×10⁴ Tcon per kilogram of body weight of the human subject, less than approximately 8.8×10⁴ Tcon per kilogram of body weight of the human subject, less than approximately 8.7×10⁴ Tcon per kilogram of body weight of the human subject, less than approximately 8.6×10⁴ Tcon per kilogram of body weight of the human subject, less than approximately 8.5×10⁴ Tcon per kilogram of body weight of the human subject, less than approximately 8.4×10⁴ Tcon per kilogram of body weight of the human subject, less than approximately 8.3×10⁴ Tcon per kilogram of body weight of the human subject, less than approximately 8.2×10⁴ Tcon per kilogram of body weight of the human subject, less than approximately 8.1×10⁴ Tcon per kilogram of body weight of the human subject, less than approximately 8.0×10⁴ Tcon per kilogram of body weight of the human subject, less than approximately 7.9×10⁴ Tcon per kilogram of body weight of the human subject, less than approximately 7.8×10⁴ Tcon per kilogram of body weight of the human subject, less than approximately 7.7×10⁴ Tcon per kilogram of body weight of the human subject, less than approximately 7.6×10⁴ Tcon per kilogram of body weight of the human subject, less than approximately 7.5×10⁴ Tcon per kilogram of body weight of the human subject, less than approximately 7.4×10⁴ Tcon per kilogram of body weight of the human subject, less than approximately 7.3×10⁴ Tcon per kilogram of body weight of the human subject, less than approximately 7.2×10⁴ Tcon per kilogram of body weight of the human subject, less than approximately 7.1×10⁴ Tcon per kilogram of body weight of the human subject, less than approximately 7.0×10⁴ Tcon per kilogram of body weight of the human subject, less than approximately 6.9×10⁴ Tcon per kilogram of body weight of the human subject, less than approximately 6.8×10⁴ Tcon per kilogram of body weight of the human subject, less than approximately 6.7×10⁴ Tcon per kilogram of body weight of the human subject, less than approximately 6.6×10⁴ Tcon per kilogram of body weight of the human subject, less than approximately 6.5×10⁴ Tcon per kilogram of body weight of the human subject, less than approximately 6.4×10⁴ Tcon per kilogram of body weight of the human subject, less than approximately 6.3×10⁴ Tcon per kilogram of body weight of the human subject, less than approximately 6.2×10⁴ Tcon per kilogram of body weight of the human subject, less than approximately 6.1×10⁴ Tcon per kilogram of body weight of the human subject, less than approximately 6.0×10⁴ Tcon per kilogram of body weight of the human subject, less than approximately 5.9×10⁴ Tcon per kilogram of body weight of the human subject, less than approximately 5.8×10⁴ Tcon per kilogram of body weight of the human subject, less than approximately 5.7×10⁴ Tcon per kilogram of body weight of the human subject, less than approximately 5.6×10⁴ Tcon per kilogram of body weight of the human subject, less than approximately 5.5×10⁴ Tcon per kilogram of body weight of the human subject, less than approximately 5.4×10⁴ Tcon per kilogram of body weight of the human subject, less than approximately 5.3×10⁴ Tcon per kilogram of body weight of the human subject, less than approximately 5.2×10⁴ Tcon per kilogram of body weight of the human subject, less than approximately 5.1×10⁴ Tcon per kilogram of body weight of the human subject, less than approximately 5.0×10⁴ Tcon per kilogram of body weight of the human subject, less than approximately 4.9×10⁴ Tcon per kilogram of body weight of the human subject, less than approximately 4.8×10⁴ Tcon per kilogram of body weight of the human subject, less than approximately 4.7×10⁴ Tcon per kilogram of body weight of the human subject, less than approximately 4.6×10⁴ Tcon per kilogram of body weight of the human subject, less than approximately 4.5×10⁴ Tcon per kilogram of body weight of the human subject, less than approximately 4.4×10⁴ Tcon per kilogram of body weight of the human subject, less than approximately 4.3×10⁴ Tcon per kilogram of body weight of the human subject, less than approximately 4.2×10⁴ Tcon per kilogram of body weight of the human subject, less than approximately 4.1×10⁴ Tcon per kilogram of body weight of the human subject, less than approximately 4.0×10⁴ Tcon per kilogram of body weight of the human subject, less than approximately 3.9×10⁴ Tcon per kilogram of body weight of the human subject, less than approximately 3.8×10⁴ Tcon per kilogram of body weight of the human subject, less than approximately 3.7×10⁴ Tcon per kilogram of body weight of the human subject, less than approximately 3.6×10⁴ Tcon per kilogram of body weight of the human subject, less than approximately 3.5×10⁴ Tcon per kilogram of body weight of the human subject, less than approximately 3.4×10⁴ Tcon per kilogram of body weight of the human subject, less than approximately 3.3×10⁴ Tcon per kilogram of body weight of the human subject, less than approximately 3.2×10⁴ Tcon per kilogram of body weight of the human subject, less than approximately 3.1×10⁴ Tcon per kilogram of body weight of the human subject, less than approximately 3.0×10⁴ Tcon per kilogram of body weight of the human subject, less than approximately 2.9×10⁴ Tcon per kilogram of body weight of the human subject, less than approximately 2.8×10⁴ Tcon per kilogram of body weight of the human subject, less than approximately 2.7×10⁴ Tcon per kilogram of body weight of the human subject, less than approximately 2.6×10⁴ Tcon per kilogram of body weight of the human subject, less than approximately 2.5×10⁴ Tcon per kilogram of body weight of the human subject, less than approximately 2.4×10⁴ Tcon per kilogram of body weight of the human subject, less than approximately 2.3×10⁴ Tcon per kilogram of body weight of the human subject, less than approximately 2.2×10⁴ Tcon per kilogram of body weight of the human subject, less than approximately 2.1×10⁴ Tcon per kilogram of body weight of the human subject, less than approximately 2.0×10⁴ Tcon per kilogram of body weight of the human subject, less than approximately 1.9×10⁴ Tcon per kilogram of body weight of the human subject, less than approximately 1.8×10⁴ Tcon per kilogram of body weight of the human subject, less than approximately 1.7×10⁴ Tcon per kilogram of body weight of the human subject, less than approximately 1.6×10⁴ Tcon per kilogram of body weight of the human subject, less than approximately 1.5×10⁴ Tcon per kilogram of body weight of the human subject, less than approximately 1.4×10⁴ Tcon per kilogram of body weight of the human subject, less than approximately 1.3×10⁴ Tcon per kilogram of body weight of the human subject, less than approximately 1.2×10⁴ Tcon per kilogram of body weight of the human subject, less than approximately 1.1×10⁴ Tcon per kilogram of body weight of the human subject, less than approximately 1.0×10⁴ Tcon per kilogram of body weight of the human subject, less than approximately 9.9×10³ Tcon per kilogram of body weight of the human subject, less than approximately 9.8×10³ Tcon per kilogram of body weight of the human subject, less than approximately 9.7×10³ Tcon per kilogram of body weight of the human subject, less than approximately 9.6×10³ Tcon per kilogram of body weight of the human subject, less than approximately 9.5×10³ Tcon per kilogram of body weight of the human subject, less than approximately 9.4×10³ Tcon per kilogram of body weight of the human subject, less than approximately 9.3×10³ Tcon per kilogram of body weight of the human subject, less than approximately 9.2×10³ Tcon per kilogram of body weight of the human subject, less than approximately 9.1×10³ Tcon per kilogram of body weight of the human subject, less than approximately 9.0×10³ Tcon per kilogram of body weight of the human subject, less than approximately 8.9×10³ Tcon per kilogram of body weight of the human subject, less than approximately 8.8×10³ Tcon per kilogram of body weight of the human subject, less than approximately 8.7×10³ Tcon per kilogram of body weight of the human subject, less than approximately 8.6×10³ Tcon per kilogram of body weight of the human subject, less than approximately 8.5×10³ Tcon per kilogram of body weight of the human subject, less than approximately 8.4×10³ Tcon per kilogram of body weight of the human subject, less than approximately 8.3×10³ Tcon per kilogram of body weight of the human subject, less than approximately 8.2×10³ Tcon per kilogram of body weight of the human subject, less than approximately 8.1×10³ Tcon per kilogram of body weight of the human subject, less than approximately 8.0×10³ Tcon per kilogram of body weight of the human subject, less than approximately 7.9×10³ Tcon per kilogram of body weight of the human subject, less than approximately 7.8×10³ Tcon per kilogram of body weight of the human subject, less than approximately 7.7×10³ Tcon per kilogram of body weight of the human subject, less than approximately 7.6×10³ Tcon per kilogram of body weight of the human subject, less than approximately 7.5×10³ Tcon per kilogram of body weight of the human subject, less than approximately 7.4×10³ Tcon per kilogram of body weight of the human subject, less than approximately 7.3×10³ Tcon per kilogram of body weight of the human subject, less than approximately 7.2×10³ Tcon per kilogram of body weight of the human subject, less than approximately 7.1×10³ Tcon per kilogram of body weight of the human subject, less than approximately 7.0×10³ Tcon per kilogram of body weight of the human subject, less than approximately 6.9×10³ Tcon per kilogram of body weight of the human subject, less than approximately 6.8×10³ Tcon per kilogram of body weight of the human subject, less than approximately 6.7×10³ Tcon per kilogram of body weight of the human subject, less than approximately 6.6×10³ Tcon per kilogram of body weight of the human subject, less than approximately 6.5×10³ Tcon per kilogram of body weight of the human subject, less than approximately 6.4×10³ Tcon per kilogram of body weight of the human subject, less than approximately 6.3×10³ Tcon per kilogram of body weight of the human subject, less than approximately 6.2×10³ Tcon per kilogram of body weight of the human subject, less than approximately 6.1×10³ Tcon per kilogram of body weight of the human subject, less than approximately 6.0×10³ Tcon per kilogram of body weight of the human subject, less than approximately 5.9×10³ Tcon per kilogram of body weight of the human subject, less than approximately 5.8×10³ Tcon per kilogram of body weight of the human subject, less than approximately 5.7×10³ Tcon per kilogram of body weight of the human subject, less than approximately 5.6×10³ Tcon per kilogram of body weight of the human subject, less than approximately 5.5×10³ Tcon per kilogram of body weight of the human subject, less than approximately 5.4×10³ Tcon per kilogram of body weight of the human subject, less than approximately 5.3×10³ Tcon per kilogram of body weight of the human subject, less than approximately 5.2×10³ Tcon per kilogram of body weight of the human subject, less than approximately 5.1×10³ Tcon per kilogram of body weight of the human subject, less than approximately 5.0×10³ Tcon per kilogram of body weight of the human subject, less than approximately 4.9×10³ Tcon per kilogram of body weight of the human subject, less than approximately 4.8×10³ Tcon per kilogram of body weight of the human subject, less than approximately 4.7×10³ Tcon per kilogram of body weight of the human subject, less than approximately 4.6×10³ Tcon per kilogram of body weight of the human subject, less than approximately 4.5×10³ Tcon per kilogram of body weight of the human subject, less than approximately 4.4×10³ Tcon per kilogram of body weight of the human subject, less than approximately 4.3×10³ Tcon per kilogram of body weight of the human subject, less than approximately 4.2×10³ Tcon per kilogram of body weight of the human subject, less than approximately 4.1×10³ Tcon per kilogram of body weight of the human subject, less than approximately 4.0×10³ Tcon per kilogram of body weight of the human subject, less than approximately 3.9×10³ Tcon per kilogram of body weight of the human subject, less than approximately 3.8×10³ Tcon per kilogram of body weight of the human subject, less than approximately 3.7×10³ Tcon per kilogram of body weight of the human subject, less than approximately 3.6×10³ Tcon per kilogram of body weight of the human subject, less than approximately 3.5×10³ Tcon per kilogram of body weight of the human subject, less than approximately 3.4×10³ Tcon per kilogram of body weight of the human subject, less than approximately 3.3×10³ Tcon per kilogram of body weight of the human subject, less than approximately 3.2×10³ Tcon per kilogram of body weight of the human subject, less than approximately 3.1×10³ Tcon per kilogram of body weight of the human subject, less than approximately 3.0×10³ Tcon per kilogram of body weight of the human subject, less than approximately 2.9×10³ Tcon per kilogram of body weight of the human subject, less than approximately 2.8×10³ Tcon per kilogram of body weight of the human subject, less than approximately 2.7×10³ Tcon per kilogram of body weight of the human subject, less than approximately 2.6×10³ Tcon per kilogram of body weight of the human subject, less than approximately 2.5×10³ Tcon per kilogram of body weight of the human subject, less than approximately 2.4×10³ Tcon per kilogram of body weight of the human subject, less than approximately 2.3×10³ Tcon per kilogram of body weight of the human subject, less than approximately 2.2×10³ Tcon per kilogram of body weight of the human subject, less than approximately 2.1×10³ Tcon per kilogram of body weight of the human subject, less than approximately 2.0×10³ Tcon per kilogram of body weight of the human subject, less than approximately 1.9×10³ Tcon per kilogram of body weight of the human subject, less than approximately 1.8×10³ Tcon per kilogram of body weight of the human subject, less than approximately 1.7×10³ Tcon per kilogram of body weight of the human subject, less than approximately 1.6×10³ Tcon per kilogram of body weight of the human subject, less than approximately 1.5×10³ Tcon per kilogram of body weight of the human subject, less than approximately 1.4×10³ Tcon per kilogram of body weight of the human subject, less than approximately 1.3×10³ Tcon per kilogram of body weight of the human subject, less than approximately 1.2×10³ Tcon per kilogram of body weight of the human subject, less than approximately 1.1×10³ Tcon per kilogram of body weight of the human subject, or less than approximately 1.0×10³ Tcon per kilogram of body weight of the human subject.

In some embodiments, a cell population of the present disclosure, for example a population of isolated CD45⁺ cells, may comprise one or more cells comprising one or more chimeric receptors. In some embodiments, the one or more cells may be T cells, NK cells, CTLs, Treg, Tmem, and/or NKT cells. In some embodiments, the one or more chimeric receptors comprise one or more extracellular antigen-binding domains that bind to one or more cancer-associated antigens. Any suitable cancer-associated antigen known in the art may be targeted. In some embodiments, the chimeric receptor is a chimeric antigen receptor or a T cell receptor.

Without wishing to be bound by theory, the cell populations of the disclosure comprise CD34⁺ cells (i.e., HSPC) and are enriched for Treg and Tmem, which contribute to positive clinical outcomes by, for example, reducing the incidence and/or severity of GVHD in a transplant recipient subject, and/or improving immune reconstitution in a transplant recipient, and/or reducing the rate of infection. Administering cell populations that comprise HSPC enriched for Treg and Tmem can facilitate retention of graft versus tumor (GVT), reduce incidence and/or severity of GVHD, and/or reduce the incidence and/or severity of infection. Without wishing to be bound by theory, administering population of cells enriched for Treg and/or Tmem, and/or depleted of naïve conventional αβ-T cells or naïve conventional CD3⁺CD25⁻CD45RA⁺ T cells (Tcon) can prevent GVHD, and/or can promote GVT effects, for example, relative to alternate hematopoietic stem cell transplantation (HCT) methods, such as standard myeloablative allogeneic hematopoietic stem cell transplant (alloHSCT), i.e., methods that are distinct from the compositions, pharmaceutical treatments, cell populations, solutions, formulations, kits, and/or methods disclosed herein. In some embodiments, administering population of cells enriched for Treg and/or Tmem, and/or depleted of naïve conventional αβ-T cells or naïve conventional CD3⁺CD25⁻CD45RA⁺ T cells (Tcon) promotes GVT effects relative to alternate HCT methods, such as standard myeloablative allogeneic hematopoietic stem cell transplant (alloHSCT), i.e., methods that are distinct from the compositions, pharmaceutical treatments, cell populations, solutions, formulations, kits, and/or methods disclosed herein.

As used herein, an alternate composition lacks one or more cell populations and/or prophylactic agent that are disclosed herein. In some embodiments, an alternate composition lacks one or more of a cell population comprising HSPC (e.g., a cell population comprising Treg), and a prophylactic agent. In some embodiments, an alternate composition or treatment regimen comprises an additional cell population or agent compared to a composition or treatment regimen of the disclosure, e.g., a an additional or different GVHD prophylactic agent.

### Acquisition, Processing, and Preparation of Cells

Certain aspects of the present disclosure relate to methods for preparing the pharmaceutical compositions, cellular therapy products, cell populations, solutions, formulations, and/or kits of the present disclosure.

In some embodiments, a biological sample (e.g., a peripheral blood donation, an umbilical cord blood donation, or a bone marrow donation) may be obtained from a donor (e.g., an HLA-matched or HLA-unmatched allogeneic donor with respect to a recipient of the present disclosure) and sorted to isolate one or more cell populations of interest such as one or more populations of HSPC, one or more populations of Treg, one or more populations of Tmem, one or more populations of iNKT, or any combination thereof. In some cases, a biological sample of the present disclosure may be contacted with a molecule that specifically binds CD34 for isolation of HSPC. The sample may be enriched for CD34⁺ cells to yield a CD34⁺ cell population and a CD34⁻ cell population. In some cases, the CD34⁻ cell population may be contacted with a molecule that specifically binds CD25 and/or a molecule that specifically binds 6B11. The sample may then be enriched for CD25⁺ and/or 6B11⁺ cells by sorting the CD34⁻ cell population for a CD25⁺ cell population, thereby yielding a CD34⁻CD25⁻ cell population or a CD34⁻CD25⁻6B11⁻ cell population, and a CD34⁻CD25⁺ cell population or a CD34⁻CD25⁺6B11⁺ cell population. The CD34⁻CD25⁺6B11⁺ cell population may be further sorted to yield a Treg and iNKT cell population. The CD34⁻CD25⁺ cells or the CD34⁻CD25⁺6B11⁺ cells may be contacted with a molecule that specifically binds CD4 and with a molecule that specifically binds CD127. The cells may then be sorted to yield a CD34⁻CD25⁺CD4⁺CD127^{dim/-} Treg cell population or a CD34⁻CD25⁺6B11⁺CD4⁺CD127^{dim/-} Treg cell population. In some cases, the CD34⁻CD25⁻ cell population or the CD34⁻CD25⁻6B11⁻ cell population may be further sorted to enrich Tmem cells. The cells may be contacted with a molecule that specifically binds CD45RA. The cell sample may be sorted to yield a CD34⁻CD25⁻CD45RA⁻ Tmem cell population or a CD34⁻CD25⁻6B11⁻CD45RA⁻ Tmem cell population. The CD45RA+naïve conventional αβ-T cells or naïve conventional CD3⁺CD25⁻CD45RA⁺ T cells (Tcon) may be discarded for the depletion of naïve conventional αβ-T cells or naïve conventional CD3⁺CD25⁻CD45RA⁺ T cells (Tcon), i.e., depletion of Tcon.

In some embodiments, at least one mobilized peripheral blood donation, at least two mobilized peripheral blood donations, or more mobilized peripheral blood donations are collected from a donor (e.g., an HLA-matched or HLA-unmatched allogeneic donor). In some embodiments, at most two mobilized peripheral blood donations are collected from the donor. In some embodiments, the mobilized peripheral blood donation is an HSPC-mobilized peripheral blood apheresis donation. In some embodiments, prior to peripheral blood donation, the donor may be vaccinated with a tumor antigen and/or a pathogen to enhance graft versus infection.

In some embodiments, at least one bone marrow donation is collected from a donor (e.g., an HLA-matched or HLA-unmatched allogeneic donor). In some embodiments, a cell population of the present disclosure is obtained from the bone marrow donation. In some embodiments at least one umbilical cord blood donation is collected from a donor (e.g., an HLA-matched or HLA-unmatched allogeneic donor). In some embodiments, a cell population of the present disclosure is obtained from the umbilical cord blood donation.

In embodiments, at least one of the mobilized peripheral blood donations, at least one of the bone marrow donations, or at least one of the umbilical cord blood donations is processed and sorted to enrich CD34+ cells, Treg, and/or Tmem. The processing and sorting for the CD34⁺ cells, Treg, and Tmem maybe done in any odder. For example, at least one of the mobilized peripheral blood donations, at least one of the bone marrow donations, or at least one of the umbilical cord blood donations may be processed and sorted to first enrich CD34⁺ cells, then Treg, and then Tmem. Alternatively, at least one of the mobilized peripheral blood donations, at least one of the bone marrow donations, or at least one of the umbilical cord blood donations may be processed and sorted to first enrich CD34⁺ cells, then Tmem, and then Treg. Alternatively, at least one of the mobilized peripheral blood donations, at least one of the bone marrow donations, or at least one of the umbilical cord blood donations may be processed and sorted to first enrich Treg, then CD34⁺ cells, and then Tmem. Alternatively, at least one of the mobilized peripheral blood donations, at least one of the bone marrow donations, or at least one of the umbilical cord blood donations may be processed and sorted to first enrich Treg, then Tmem, and then CD34⁺ cells. Alternatively, at least one of the mobilized peripheral blood donations, at least one of the bone marrow donations, or at least one of the umbilical cord blood donations may be processed and sorted to first enrich Tmem, then CD34⁺ cells, and then Treg. Alternatively, at least one of the mobilized peripheral blood donations, at least one of the bone marrow donations, or at least one of the umbilical cord blood donations may be processed and sorted to first enrich Tmem, then Treg, and then CD34⁺ cells.

In some embodiments, the processing and sorting time of the at least one of the mobilized peripheral blood donations, at least one of the bone marrow donations, or at least one of the umbilical cord blood donations is less than about 40 hours, the processing and sorting time of the at least one of the mobilized peripheral blood donations, at least one of the bone marrow donations, or at least one of the umbilical cord blood donations is less than about 35 hours, the processing and sorting time of the at least one of the mobilized peripheral blood donations, at least one of the bone marrow donations, or at least one of the umbilical cord blood donations is less than about 30 hours, the processing and sorting time of the at least one of the mobilized peripheral blood donations, at least one of the bone marrow donations, or at least one of the umbilical cord blood donations is less than about 25 hours, the processing and sorting time of the at least one of the mobilized peripheral blood donations, at least one of the bone marrow donations, or at least one of the umbilical cord blood donations is less than about 20 hours, the processing and sorting time of the at least one of the mobilized peripheral blood donations, at least one of the bone marrow donations, or at least one of the umbilical cord blood donations is less than about 15 hours.

In some embodiments, the processing and sorting time of the at least one of the mobilized peripheral blood donations, at least one of the bone marrow donations, or at least one of the umbilical cord blood donations is at most about 35 hours, the processing and sorting time of the at least one of the mobilized peripheral blood donations, at least one of the bone marrow donations, or at least one of the umbilical cord blood donations is at most about 30 hours, the processing and sorting time of the at least one of the mobilized peripheral blood donations, at least one of the bone marrow donations, or at least one of the umbilical cord blood donations is at most about 25 hours, the processing and sorting time of the at least one of the mobilized peripheral blood donations, at least one of the bone marrow donations, or at least one of the umbilical cord blood donations is at most about 20 hours, or the processing and sorting time of the at least one of the mobilized peripheral blood donations, at least one of the bone marrow donations, or at least one of the umbilical cord blood donations is at most about 15 hours.

In some embodiments, a cell population of the present disclosure (e.g., a population of isolated CD45⁺ cells) is obtained from whole blood. A cell population of the present disclosure can be obtained from a peripheral blood apheresis product, for example, a mobilized peripheral blood apheresis product, e.g., mobilized by administration of one or more mobilization agents to a donor. Non-limiting examples of mobilization agents include granulocyte colony-stimulating factor (G-CSF), granulocyte macrophage colony-stimulating factor (GM-CSF), stem cell factor (SCF), a SDF-1 antagonist, a CXCR4 antagonist (e.g., POL6326, BKT-140, TG-0054, NOX-A12), MOZOBIL® (plerixafor), a CXCR2 ligand (e.g., GROP), a sphingosine-1-phosphatase (SIP) agonist. (e.g., SEW2871), a VCAM/VLA-4 inhibitor (e.g., BIO5192), a proteosome inhibitor (e.g., Bortezomib), parathyroid hormone, a hypoxia inducible factor (HIF) stabilizer (e.g., FG-4497), and combinations thereof. Techniques to mobilize stem cells into peripheral blood can comprise administering to a donor, for example, 10 to 40 g/kg/day of a mobilization agent. A mobilization agent can be administered to the donor in, for example, 1, 2, 3, 4, 5, 6, 7, 8, 9, or 10 doses. An apheresis product can be isolated from a donor about, for example, 1, 2, 3, 4, 5, 6, 7, 8, 9, 10, 11, 12, 13, 14, 15, 16, 17, 18, 19, 20, 21, 22, 23, 24, 26, 28, or 30 hour(s) after a dose of mobilization agent.

A cell population of the present disclosure can be obtained from at least one apheresis product, two apheresis products, three apheresis products, four apheresis products, five apheresis products, six apheresis products, or more. In some embodiments, a cell population of the present disclosure is obtained from one apheresis product. In some embodiments, a cell population of the present disclosure is obtained from two apheresis products. In some embodiments, a cell population of the present disclosure is obtained from an apheresis product from one donor and an apheresis product from an at least second donor. In some embodiments, a cell population of the present disclosure can be obtained from more than one apheresis products obtained from the donor on different days. In some embodiments, a bone marrow stimulant (e.g., plerixafor 0.24 mg/kg subcutaneously) can be administered to the donor after the first apheresis and prior to subsequent apheresis.

A cell population of the present disclosure can be refined by selection from a population of cells, for example, peripheral blood or a peripheral blood apheresis product, bone marrow, or umbilical cord blood. Selection methods for cell populations can comprise methods involving positive or negative selection of a cell population of interest. Selection methods for cell populations can comprise affinity reagents, including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a polyaminoacid. In some embodiments, the affinity reagent is directly conjugated to a detection reagent and/or purification reagent. In some cases, the detection reagent and purification reagent are the same. In some cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system (CliniMACs) of columns, antibodies, buffers, preparation materials and reagents.

Affinity reagents can comprise immunoaffinity reagents, utilizing the binding specificity of antibodies or fragments or derivatives thereof to positively or negatively select for a cell population of interest. Selection methods for cell populations can comprise an affinity agent and a column, such as magnetic activated cell sorting (MACS) with specific antibodies and microbeads. Selection methods for cell populations can comprise fluorescent activated cell sorting (FACS), with cell populations sorted based on staining profiles with one or more fluorescently-conjugated antibodies. Selection methods for cell populations can comprise physical adsorption, for example, physical adsorption of T cells to protein ligands such as lectins.

The number of cells in the various cell populations provided herein can be determined, for example, by quantifying the cell determinants via flow cytometry. In some embodiments, dose calculations are adjusted based on measures of cell viability measurements, e.g., viability determined via flow cytometry with propidium iodide or 7-AAD, or via trypan blue exclusion.

In various embodiments, the at least one mobilized peripheral blood donation, at least one bone marrow donation, and/or at least one umbilical cord blood donation is processed and sorted using one or more immune-separation particles (ISPs), e.g., ISPs comprise affinity reagents such as immuno-magnetic separation particles which may be antibodies each conjugated to an iron-containing particle. In some embodiments, the affinity reagents comprise a plurality of CD34-reagents (e.g., an anti-CD34 antibody) that binds to one or more CD34 receptors on a HSPC.

In some cases, at least a portion of the plurality of ISPs are attached to CD34+ receptors on the HSPC's of the HSPC cell population; optionally, an average number of ISP's per HSPC in the HSPC cell population is less than about 20,000, an average number of ISP's per HSPC in the HSPC cell population is equal to or less than about 10,000, and/or an average number of ISP's per HSPC in the HSPC cell population is from about 1000 to about 20,000.

In some embodiments, an average number of ISP's per HSPC in the HSPC cell population may be about 1,500 to about 20,000. In some embodiments, an average number of ISP's per HSPC in the HSPC cell population may be at least about 1,500. In some embodiments, an average number of ISP's per HSPC in the HSPC cell population may be at most about 20,000. In some embodiments, an average number of ISP's per HSPC in the HSPC cell population may be about 1,500 to about 2,000, about 1,500 to about 5,000, about 1,500 to about 6,000, about 1,500 to about 10,000, about 1,500 to about 12,000, about 1,500 to about 15,000, about 1,500 to about 20,000, about 2,000 to about 5,000, about 2,000 to about 6,000, about 2,000 to about 10,000, about 2,000 to about 12,000, about 2,000 to about 15,000, about 2,000 to about 20,000, about 5,000 to about 6,000, about 5,000 to about 10,000, about 5,000 to about 12,000, about 5,000 to about 15,000, about 5,000 to about 20,000, about 6,000 to about 10,000, about 6,000 to about 12,000, about 6,000 to about 15,000, about 6,000 to about 20,000, about 10,000 to about 12,000, about 10,000 to about 15,000, about 10,000 to about 20,000, about 12,000 to about 15,000, about 12,000 to about 20,000, or about 15,000 to about 20,000. In some embodiments, an average number of ISP's per HSPC in the HSPC cell population may be about 1,500, about 2,000, about 5,000, about 6,000, about 10,000, about 12,000, about 15,000, or about 20,000. In some embodiments, an average number of ISP's per HSPC in the HSPC cell population may be at least 1,500, 2,000, 5,000, 6,000, 10,000, 12,000, 15,000, or 20,000. In some embodiments, an average number of ISP's per HSPC in the HSPC cell population may be at most 1,500, 2,000, 5,000, 6,000, 10,000, 12,000, 15,000, or 20,000.

In some cases, at least a portion of the plurality of ISPs are attached to CD25+ receptors on the cells of the Treg cell population; optionally, an average number of ISP's per T-reg cell in the Treg population is equal or less than about 4000 or an average number of ISPs per T-reg cell in the Treg population is from about 1500 to about 2500. In some cases, at least a portion of the plurality of ISPs are attached to CD3+ receptors on the cells of the heterogenous cell population; optionally, an average number of ISPs per cell in population of T heterogenous is less than about 4,000.

In some embodiments, an average number of ISP's per Treg cells in the Treg cell population may be about 500 to about 4,000. In some embodiments, an average number of ISP's per Treg cells in the Treg cell population may be at least about 500. In some embodiments, an average number of ISP's per Treg cells in the Treg cell population may be at most about 4,000. In some embodiments, an average number of ISP's per Treg cells in the Treg cell population may be about 500 to about 1,000, about 500 to about 1,500, about 500 to about 2,000, about 500 to about 2,500, about 500 to about 3,000, about 500 to about 4,000, about 1,000 to about 1,500, about 1,000 to about 2,000, about 1,000 to about 2,500, about 1,000 to about 3,000, about 1,000 to about 4,000, about 1,500 to about 2,000, about 1,500 to about 2,500, about 1,500 to about 3,000, about 1,500 to about 4,000, about 2,000 to about 2,500, about 2,000 to about 3,000, about 2,000 to about 4,000, about 2,500 to about 3,000, about 2,500 to about 4,000, or about 3,000 to about 4,000. In some embodiments, an average number of ISP's per Treg cells in the Treg cell population may be about 500, about 1,000, about 1,500, about 2,000, about 2,500, about 3,000, or about 4,000. In some embodiments, an average number of ISP's per Treg cells in the Treg cell population may be at least 500, 1,000, 1,500, 2,000, 2,500, 3,000, or 4,000. In some embodiments, an average number of ISP's per Treg cells in the Treg cell population may be at most 500, 1,000, 1,500, 2,000, 2,500, 3,000, or 4,000.

A cell population of the present disclosure can be enriched for Treg. Treg can be selected based on expression of markers including CD3, CD4, CD25, CD127, FOXP3, and combinations thereof. Treg can be isolated from whole blood. Treg can be isolated from a peripheral blood apheresis product, from bone marrow, and/or from umbilical cord blood. Treg can be isolated from a population of cells previously enriched and/or depleted for one or more other cell types, e.g., isolated from a population of cells depleted of CD34+ cells. In some embodiments, Treg are isolated from the flow-through fraction of a CD34+ MACS selection. The number of Treg in a population of cells can be determined, for example, by flow cytometry, where Treg can be identified as, for example, CD4+CD25+CD127dim or CD4+FOXP3+. Dose calculations can be adjusted based on measures of cell viability measurements, e.g., viability determined via flow cytometry with propidium iodide or 7-AAD, or via trypan blue exclusion.

A cell population of the present disclosure can comprise a population of Tmem. A population of Tmem can be sourced from peripheral blood, bone marrow, and/or umbilical cord blood. A population of Tmem can be sourced from a peripheral blood apheresis product. A population of cells can be enriched for Tmem, for example, by sorting based on the expression of various markers using MACS, FACS, or a combination thereof. A population of Tmem can be enriched, for example, by sorting for CD3+CD45RA-CD45RO+ cells. The number of Tmem present in a population can be quantified, for example, by quantifying CD3+CD45RA-CD45RO+ cells via flow cytometry. The number of CD3+CD45RA-CD45RO+ cells in an aliquot can be determined and a volume comprising an appropriate dose of Tmem administered to the recipient. Dose calculations can be adjusted based on measures of cell viability measurements, e.g., viability determined via flow cytometry with propidium iodide or 7-AAD, or via trypan blue exclusion.

A cell population of the disclosure can comprise a population of iNKT. A population of iNKT can be sourced from peripheral blood, bone marrow, and/or umbilical cord blood. A population of iNKT can be sourced from a peripheral blood apheresis product. A population of cells can be enriched for iNKT, for example, by sorting based on the expression of various markers using MACS, FACS, or a combination thereof. A population of iNKT can be enriched, for example, by sorting for CD3+Vα24Jα18+ cells. The number of iNKT present in a population can be quantified, for example, by quantifying CD3+Vα24Jα18⁺ cells via flow cytometry. The number of CD3+Vα24Jα18+ cells in an aliquot can be determined and a volume comprising an appropriate dose of iNKT administered to the recipient. In some embodiments, dose calculations are adjusted based on measures of cell viability measurements, e.g., viability determined via flow cytometry with propidium iodide or 7-AAD, or via trypan blue exclusion.

A cell population of the present disclosure can lack or have reduced amounts of naïve conventional αβ-T cells or naïve conventional CD3⁺CD25⁻CD45RA⁺ T cells (Tcon). In some embodiments, the Tcon are CD45RA⁺. These cells may be discarded to result in a depletion of naïve conventional αβ-T cells or naïve conventional CD3⁺CD25⁻CD45RA⁺ T cells (Tcon).

A cell population of the present disclosure can be administered freshly after isolation. Cells freshly isolated from a donor (“fresh cells”) can be administered to a recipient subject. Fresh cells can be stored in a buffer, for example, CliniMACs PBS-EDTA Buffer with 0.5% human serum albumin, or Plasma-Lyte-A, pH 7.4 supplemented with 2% human serum albumin. Fresh cells can be stored at a reduced temperature (e.g., 2-8° C.), and without being cryopreserved/frozen.

After acquiring a fresh population of cells from a donor, the fresh cells can be stored for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 44 hours, at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to administration to a subject.

After acquiring a fresh population of cells from a donor, the fresh cells can be stored for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 16 hours, at most about 18 hours, at most about 20, at most 22 hours, at most about 24 hours, at most about 30 hours, at most about 36 hours, at most about 40 hours, at most about 48 hours, at most about 60 hours, at most about 70 hours, at most about 72 hours, at most about 80 hours, at most about 90 hours, at most about 96 hours, at most about 120 hours, at most about 150 hours, at most about 200 hours, or at most about 300 hours prior to administration to a subject.

In some embodiments, after processing, one or more cell populations of the present disclosure (e.g., a population of isolated CD45⁺ cells) may be formulated with one or more excipients. In some embodiments, the one or more excipients comprise buffers, such as transport or infusion buffers. In some embodiments, the cell populations are formulated at a neutral pH. In some embodiments, the population of HSPC, or the at least one dose of HSPC, of the present disclosure is formulated with one or more excipients at a neutral pH. In some embodiments, the population of Treg, or the at least one dose of Treg, of the present disclosure is formulated with one or more excipients at a neutral pH. In some embodiments, the population of Tmem, or the at least one dose of Tmem, of the present disclosure are formulated with one or more excipients at a neutral pH. In some embodiments, the population of iNKT, or the at least one dose of iNKT, of the present disclosure are formulated with one or more excipients at a neutral pH. In some embodiments, the population or dose of HSPC and the population or dose of Treg of the present disclosure are formulated together with one or more excipients at a neutral pH. In some embodiments, the population or dose of HSPC and the population or dose of Tmem of the present disclosure are formulated together with one or more excipients at a neutral pH. In some embodiments, the population or dose of HSPC and the population or dose of iNKT of the present disclosure are formulated together with one or more excipients at a neutral pH. In some embodiments, the population or dose of Treg and the population or dose of Tmem of the present disclosure are formulated together with one or more excipients at a neutral pH. In some embodiments, the population or dose of Treg and the population or dose of iNKT of the present disclosure are formulated together with one or more excipients at a neutral pH. In some embodiments, the population or dose of Tmem and the population or dose of iNKT of the present disclosure are formulated together with one or more excipients at a neutral pH. In some embodiments, the population or dose of HSPC, the population or dose of Treg, and the population or dose of Tmem of the present disclosure are formulated together with one or more excipients at a neutral pH. In some embodiments, the population or dose of HSPC, the population or dose of Treg, and the population or dose of iNKT of the present disclosure are formulated together with one or more excipients at a neutral pH. In some embodiments, the population or dose of HSPC, the population or dose of Tmem, and the population or dose of iNKT of the present disclosure are formulated together with one or more excipients at a neutral pH. In some embodiments, the population or dose of Treg, the population or dose of Tmem, and the population or dose of iNKT of the present disclosure are formulated together with one or more excipients at a neutral pH. In some embodiments, the population or dose of HSPC, the population or dose of Treg, the population or dose of Tmem, and the population or dose of iNKT of the present disclosure are formulated together with one or more excipients at a neutral pH. In some embodiments, the one or more excipients comprise one or more transport buffers.

In some embodiments, a neutral pH ranges from approximately 6.8 to approximately 7.6. In some embodiments, a neutral pH is approximately 6.8, approximately 6.85, approximately 6.9, approximately 6.95, approximately 7, approximately 7.05, approximately 7.1, approximately 7.15, approximately 7.2, approximately 7.25, approximately 7.3, approximately 7.35, approximately 7.4, approximately 7.45, approximately 7.5, approximately 7.55, or approximately 7.6.

In some embodiments, one or more cell populations of the present disclosure (e.g., a population of isolated CD45⁺ cells) may be formulated in one or more excipients. In some embodiments, the one or more excipients may comprise one or more buffers. The buffers may be transport buffers or infusion buffers. Transport buffers suitable for use with the cell populations of the present disclosure include buffers that have a milliequivalent (mEq) of sodium that ranges from approximately 120 mEq sodium to approximately 160 mEq sodium. In some embodiments, a transport buffer of the present disclosure comprises approximately 120 mEq sodium, approximately 125 mEq sodium, approximately 130 mEq sodium, approximately 135 mEq sodium, approximately 140 mEq sodium, approximately 145 mEq sodium, approximately 120 mEq sodium, approximately 150 mEq sodium, approximately 155 mEq sodium, or approximately 160 mEq sodium.

Transport buffers suitable for use with the cell populations of the present disclosure include buffers that have an osmotic concentration that ranges from approximately 270 mOsmol/L to approximately 320 mOsmol/L. In some embodiments, a transport buffer of the present disclosure have an osmotic concentration that is approximately 270 mOsmol/L, approximately 275 mOsmol/L, approximately 280 mOsmol/L, approximately 285 mOsmol/L, approximately 290 mOsmol/L, approximately 295 mOsmol/L, approximately 300 mOsmol/L, approximately 305 mOsmol/L, approximately 310 mOsmol/L, approximately 315 mOsmol/L, or approximately 320 mOsmol/L.

In some embodiments, transport buffers suitable for use with one or more cell populations of the present disclosure (e.g., a population of isolated CD45⁺ cells) include, without limitation, phosphate-buffered saline (PBS), human serum, PlasmaLyte, and any combination thereof. In some embodiments, one or more cell populations of the present disclosure (e.g., a population of isolated CD45⁺ cells) are formulated with a transport buffer. In some embodiments, one or more cell populations of the present disclosure (e.g., a population of isolated CD45⁺ cells) are formulated with a sufficient amount of PBS. In some embodiments, one or more cell populations of the present disclosure (e.g., a population of isolated CD45⁺ cells) are formulated with a sufficient amount of human serum. In some embodiments, one or more cell populations of the present disclosure (e.g., a population of isolated CD45⁺ cells) are formulated with a sufficient amount of PlasmaLyte (e.g., PlasmaLyte A).

In some embodiments, a transport buffer of the present disclosure may further include a human carrier protein, including without limitation, human serum albumin (HSA), intravenous immune globulin (IVIG), AB serum, or any combination thereof. In some embodiments, the human carrier protein has a concentration that ranges from approximately 0.10% weight by volume to approximately 10% weight by volume of the human carrier protein. In some embodiments, the human carrier protein has a concentration that is approximately 0.1% weight by volume, approximately 0.2% weight by volume, approximately 0.3% weight by volume, approximately 0.4% weight by volume, approximately 0.5% weight by volume, approximately 0.6% weight by volume, approximately 0.7% weight by volume, approximately 0.8% weight by volume, approximately 0.9 weight by volume, approximately 1.0% weight by volume, approximately 1.10% weight by volume, approximately 1.2% weight by volume, approximately 1.3% weight by volume, approximately 1.4% weight by volume, approximately 1.5% weight by volume, approximately 1.6% weight by volume, approximately 1.7% weight by volume, approximately 1.8% weight by volume, approximately 1.9% weight by volume, approximately 2.0% weight by volume, approximately 2.1% weight by volume, approximately 2.2% weight by volume, approximately 2.3% weight by volume, approximately 2.4% weight by volume, approximately 2.5% weight by volume, approximately 2.6% weight by volume, approximately 2.7% weight by volume, approximately 2.8% weight by volume, approximately 2.9% weight by volume, approximately 3.0% weight by volume, approximately 3.1% weight by volume, approximately 3.2% weight by volume, approximately 3.3% weight by volume, approximately 3.4% weight by volume, approximately 3.5% weight by volume, approximately 3.6% weight by volume, approximately 3.7% weight by volume, approximately 3.8% weight by volume, approximately 3.9% weight by volume, approximately 4.0% weight by volume, approximately 4.1% weight by volume, approximately 4.2% weight by volume, approximately 4.3% weight by volume, approximately 4.4% weight by volume, approximately 4.5% weight by volume, approximately 4.6% weight by volume, approximately 4.7% weight by volume, approximately 4.8% weight by volume, approximately 4.9% weight by volume, approximately 5.0% weight by volume, approximately 5.1% weight by volume, approximately 5.2% weight by volume, approximately 5.3% weight by volume, approximately 5.4% weight by volume, approximately 5.5% weight by volume, approximately 5.6% weight by volume, approximately 5.7% weight by volume, approximately 5.8% weight by volume, approximately 5.9% weight by volume, approximately 6.0% weight by volume, approximately 6.1% weight by volume, approximately 6.2% weight by volume, approximately 6.3% weight by volume, approximately 6.4% weight by volume, approximately 6.5% weight by volume, approximately 6.6% weight by volume, approximately 6.7% weight by volume, approximately 6.8% weight by volume, approximately 6.9% weight by volume, approximately 7.0% weight by volume, approximately 7.1% weight by volume, approximately 7.2% weight by volume, approximately 7.3% weight by volume, approximately 7.4% weight by volume, approximately 7.5% weight by volume, approximately 7.6% weight by volume, approximately 7.7% weight by volume, approximately 7.8% weight by volume, approximately 7.9% weight by volume, approximately 8.0% weight by volume, approximately 8.1% weight by volume, approximately 8.2% weight by volume, approximately 8.3% weight by volume, approximately 8.4% weight by volume, approximately 8.5% weight by volume, approximately 8.6% weight by volume, approximately 8.7% weight by volume, approximately 8.8% weight by volume, approximately 8.9% weight by volume, approximately 9.0% weight by volume, approximately 9.1% weight by volume, approximately 9.2% weight by volume, approximately 9.3% weight by volume, approximately 9.4% weight by volume, approximately 9.5% weight by volume, approximately 9.6% weight by volume, approximately 9.7% weight by volume, approximately 9.8% weight by volume, approximately 9.9% weight by volume, or approximately 10% weight by volume of the human carrier protein.

In some embodiments, one or more cell populations of the present disclosure (e.g., a population of isolated CD45⁺ cells) are formulated with an excipient, such as a transport buffer of the present disclosure, in an amount sufficient to yield a volume that ranges from approximately 5 mL to 1 L. In some embodiments, the population of cells is formulated with an excipient, such as a transport buffer of the present disclosure, in an amount sufficient to yield a volume that ranges from approximately 5 mL to 1 L. In some embodiments, the population of cells is formulated (e.g., with one or more excipients, such as a transport buffer) at volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 105 mL, approximately 110 mL, approximately 115 mL, approximately 120 mL, approximately 125 mL, approximately 130 mL, approximately 135 mL, approximately 140 mL, approximately 145 mL, approximately 150 mL, approximately 155 mL, approximately 160 mL, approximately 165 mL, approximately 170 mL, approximately 175 mL, approximately 180 mL, approximately 185 mL, approximately 190 mL, approximately 195 mL, approximately 200 mL, approximately 205 mL, approximately 210 mL, approximately 215 mL, approximately 220 mL, approximately 225 mL, approximately 230 mL, approximately 235 mL, approximately 240 mL, approximately 245 mL, approximately 250 mL, approximately 255 mL, approximately 260 mL, approximately 265 mL, approximately 270 mL, approximately 275 mL, approximately 280 mL, approximately 285 mL, approximately 290 mL, approximately 295 mL, approximately 300 mL, approximately 305 mL, approximately 310 mL, approximately 315 mL, approximately 320 mL, approximately 325 mL, approximately 330 mL, approximately 335 mL, approximately 340 mL, approximately 345 mL, approximately 350 mL, approximately 355 mL, approximately 360 mL, approximately 365 mL, approximately 370 mL, approximately 375 mL, approximately 380 mL, approximately 385 mL, approximately 390 mL, approximately 395 mL, approximately 400 mL, approximately 405 mL, approximately 410 mL, approximately 415 mL, approximately 420 mL, approximately 425 mL, approximately 430 mL, approximately 435 mL, approximately 440 mL, approximately 445 mL, approximately 450 mL, approximately 455 mL, approximately 460 mL, approximately 465 mL, approximately 470 mL, approximately 475 mL, approximately 480 mL, approximately 485 mL, approximately 490 mL, approximately 495 mL, approximately 500 mL, approximately 505 mL, approximately 510 mL, approximately 515 mL, approximately 520 mL, approximately 525 mL, approximately 530 mL, approximately 535 mL, approximately 540 mL, approximately 545 mL, approximately 550 mL, approximately 555 mL, approximately 560 mL, approximately 565 mL, approximately 570 mL, approximately 575 mL, approximately 580 mL, approximately 585 mL, approximately 590 mL, approximately 595 mL, approximately 600 mL, approximately 605 mL, approximately 610 mL, approximately 615 mL, approximately 620 mL, approximately 625 mL, approximately 630 mL, approximately 635 mL, approximately 640 mL, approximately 645 mL, approximately 650 mL, approximately 655 mL, approximately 660 mL, approximately 665 mL, approximately 670 mL, approximately 675 mL, approximately 680 mL, approximately 685 mL, approximately 690 mL, approximately 695 mL, approximately 700 mL, approximately 705 mL, approximately 710 mL, approximately 715 mL, approximately 720 mL, approximately 725 mL, approximately 730 mL, approximately 735 mL, approximately 740 mL, approximately 745 mL, approximately 750 mL, approximately 755 mL, approximately 760 mL, approximately 765 mL, approximately 770 mL, approximately 775 mL, approximately 780 mL, approximately 785 mL, approximately 790 mL, approximately 795 mL, approximately 800 mL, approximately 805 mL, approximately 810 mL, approximately 815 mL, approximately 820 mL, approximately 825 mL, approximately 830 mL, approximately 835 mL, approximately 840 mL, approximately 845 mL, approximately 850 mL, approximately 855 mL, approximately 860 mL, approximately 865 mL, approximately 870 mL, approximately 875 mL, approximately 880 mL, approximately 885 mL, approximately 890 mL, approximately 895 mL, approximately 900 mL, approximately 905 mL, approximately 910 mL, approximately 915 mL, approximately 920 mL, approximately 925 mL, approximately 930 mL, approximately 935 mL, approximately 940 mL, approximately 945 mL, approximately 950 mL, approximately 955 mL, approximately 960 mL, approximately 965 mL, approximately 970 mL, approximately 975 mL, approximately 980 mL, approximately 985 mL, approximately 990 mL, approximately 995 mL, or approximately 1 L. In some embodiments, a formulation of the present disclosure further comprises a human carrier protein of the present disclosure. In some embodiments, a formulation of the present disclosure further comprises one or more cryoprotectants.

Cells populations of the present disclosure (e.g., a population of isolated CD45⁺ cells) can be cryopreserved. In some embodiments, cryopreservation can be beneficial to the methods disclosed herein. Cryopreservation can comprise addition of a preservative agent (e.g., DMSO) or one or more cryoprotectants, and gradual cooling of cells in a controlled-rate freezer to prevent osmotic cellular injury resulting from ice crystal formation. Cryopreservation can comprise commercial cryopreservation reagents and materials (e.g., cryoprotectants), for example, Cryobags sorbitol, dimethyl sulfoxide (DMSO), propylene glycol, glycerol, polyvinylpyrrolidone (PVP), and polyethylene glycol (PEG), serum, HSA, hetastarch, CRYOSTOR CS2, CRYOSTOR CS5, and CRYOSTOR CS10, or any combination thereof. In certain embodiments, a cryoprotectant of the present disclosure comprises DMSO. For example, in some embodiments the population of cells may be formulated for cryopreservation with a cryoprotectant that comprises DMSO in an amount that ranges from approximately 1% volume by volume to approximately 15% volume by volume. Cryopreserved cells can be stored for periods of time ranging from hours to years at low temperatures. Cryopreserved cells can be stored at ultralow temperatures, for example, −50° C., −60° C., −70° C., −80° C., −90° C., −100° C., −110° C., −120° C., −130° C., −140° C., −150° C., −160° C., −170° C., −180° C., −190° C., −196° C., or less. Cryopreserved cells can be stored in storage devices comprising liquid nitrogen. Cells can be cryopreserved before or after certain steps in the methods of the disclosure, for example, before or after sorting steps, before or after characterization steps, such as determining cell viability or the concentration of cells of a particular type.

In some embodiments, once formulated, one or more cell populations of the present disclosure (e.g., a population of isolated CD45⁺ cells) may be placed into one or more containers. In certain aspects, the present disclosure related to a system comprising one or more containers containing one or more cell populations of the present disclosure (e.g., a population of isolated CD45⁺ cells). In some embodiments, the one or more containers are filled to an appropriate volume (e.g., 5 mL to 1 L) with one or more excipients of the present disclosure, such as one or more transport buffers, one or more human carrier proteins, and/or one or more cryoprotectants, at a suitable pH, e.g., a neutral pH. In some embodiments, a single container contains the population of cells (e.g., a population of isolated CD45⁺ cells). In some embodiments, a container of the present disclosure includes, without limitation, a vial, a syringe, a bag (e.g., transfer bag), a cryoprotectant container, and any combinations thereof. In some embodiments, the container is a bag, such as a transfer bag, that can be used, for example, for infusion. In some embodiments, a transfer bag of the present disclosure is a single dose transfer bag. In some embodiments, a transfer bag of the present disclosure is a polyvinyl chloride (PVC) transfer bag or an ethylene vinyl acetate (EVA) transfer bag.

Certain aspects of the present disclosure relate to systems, e.g., a cellular therapy or treatment system that comprises one or more of the containers of the present disclosure. Other aspects of the present disclosure relate to articles of manufacture comprising one or more of the containers of the present disclosure. Other aspects of the present disclosure relate to kits comprising one or more of the containers of the present disclosure.

### Donors

### 1. General Information

Human leukocyte antigens (HLA), also broadly referred to as Major histocompatibility complex (MHC) antigens, can be protein molecules expressed on the surface of a cell that can confer an antigenic identity to that cell. HLA/MHC antigens are target molecules that can be recognized by T cells and natural killer (NK) cells as being derived from the same source of hematopoietic stem cells as the immune effector cells (“self”), or as being derived from another source of hematopoietic cells (“non-self”). HLA class I antigens (A, B, and C in humans) can be expressed by the vast majority of cells, while HLA class II antigens (DR, DP, and DQ in humans) can be expressed primarily on professional antigen presenting cells. Both HLA classes can be implicated in GVHD.

HLA antigens are encoded by highly polymorphic genes; a range of alleles exist for each HLA class I and II gene. Allelic gene products can differ in one or more amino acids in the α and/or β domain(s). Panels of specific antibodies or nucleic acid reagents can be used to determine HLA haplotypes of individuals, for example, using leukocytes that express class I and class II molecules. HLA alleles can be described at various levels of detail. Most designations begin with HLA- and the locus name, then * and some (even) number of digits specifying the allele. The first two digits can specify a group of alleles. The third through fourth digits, when present, can specify a synonymous allele. Digits five through six, when present, can denote any synonymous mutations within the coding frame of the gene. The seventh and eighth digits, when present, can distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

The set of HLA alleles inherited from one parent forms a haplotype. HLA haploidentical can refer to a donor-recipient pair where one chromosome is matched at least at HLA-A; HLA-B, and HLA-DR between the donor and recipient. The haploidentical pair may or may not be matched at other alleles, e.g., other HLA genes on the other chromosome, or additional histocompatibility loci on either chromosome. Such donors can frequently occur in families, e.g., a parent can be haploidentical to a child; and siblings may be haploidentical.

A cell population can be from a donor that has been HLA-typed at any number of HLA alleles. A donor and a subject can be HLA matched, e.g., matched at all typed HLA alleles. A donor and a subject can be HLA mismatched, e.g., at least one HLA antigen can be mismatched between the donor and recipient.

In some embodiments, a related or unrelated donor and a subject can be HLA-typed at six alleles consisting of HLA-A, HLA-B, and HLA-DR alleles. The donor and subject can be matched at, for example 3/6 4/6, 5/6, or 6/6 of the alleles. In some embodiments, the donor and subject are matched at least at 5/6 alleles. In some embodiments, the donor and subject are matched at 6/6 alleles.

In some embodiments, a related or unrelated donor and a subject can be HLA-typed at eight alleles consisting of HLA-A, HLA-B, HLA-C, and HLA-DR alleles (e.g., HLA-DRB1 alleles). The donor and subject can be matched at, for example 4/8, 5/8, 6/8, 7/8, or 8/8 of the alleles. In some embodiments, the donor and subject are matched at least at 6/8 alleles. In some embodiments, the donor and subject are matched at least at 7/8 alleles. In some embodiments, the donor and subject are matched at 8/8 alleles.

In some embodiments, a related or unrelated donor and a subject can be HLA-typed at ten alleles consisting of HLA-A, HLA-B, HLA-C, and HLA-DR alleles (e.g., HLA-DRB1 alleles). The donor and subject can be matched at, for example 5/10, 6/10, 7/10, 8/10, 9/10, or 10/10 of the alleles. In some embodiments, the donor and subject are matched at least at 7/10 alleles. In some embodiments, the donor and subject are matched at least at 8/10 alleles. In some embodiments, the donor and subject are matched at least at 9/10 alleles. In some embodiments, the donor and subject are matched at 10/10 alleles.

In some embodiments, a related or unrelated donor and a subject can be HLA-typed at twelve alleles, for example, HLA-A, HLA-B, HLA-C, HLA-DR alleles (e.g., HLA-DRB1 alleles), and HLA-DP alleles (e.g., HLA-DPB1 alleles). The donor and subject can be matched at, for example 6/12, 7/12, 8/12, 9/12, 10/12, 11/12, or 12/12 of the alleles. In some embodiments, the donor and subject are matched at least at 9/12 alleles. In some embodiments, the donor and subject are matched at least at 10/12 alleles. In some embodiments, the donor and subject are matched at least at 11/12 alleles. In some embodiments, the donor and subject are matched at 12/12 alleles.

A cell population can be generated from a matched unrelated donor that is an 8/8 match for HLA-A, -B, -C, and -DRB1, all typed using DNA-based high-resolution methods. A cell population can be generated from a matched unrelated donor that is an 7/8 match for HLA-A, -B, -C, and -DRB1, all typed using DNA-based high-resolution methods. A cell population can be generated from a matched unrelated donor that is an 6/8 match for HLA-A, -B, -C, and -DRB1, all typed using DNA-based high-resolution methods. A cell population can be generated from a matched unrelated donor that is an 10/10 match for HLA-A, -B, -C, -DQB1 and -DRB1, all typed using DNA-based high-resolution methods. A cell population can be generated from a matched unrelated donor that is an 9/10 match for HLA-A, -B, -C, -DQB1 and -DRB1, all typed using DNA-based high-resolution methods. A cell population can be generated from a matched unrelated donor that is an 8/10 match for HLA-A, -B, -C, -DQB1 and -DRB1, all typed using DNA-based high-resolution methods. A cell population can be generated from a matched unrelated donor that is an 7/10 match for HLA-A, -B, -C, -DQB1 and -DRB1, all typed using DNA-based high-resolution methods. A cell population can be generated from a matched unrelated donor that is an 12/12 match for HLA-A, -B, -C, -DQB1, -DRB1, and -DPB1, all typed using DNA-based high-resolution methods. A cell population can be generated from a matched unrelated donor that is an 11/12 match for HLA-A, -B, -C, -DQB1, -DRB1, and -DPB1, all typed using DNA-based high-resolution methods. A cell population can be generated from a matched unrelated donor that is an 10/12 match for HLA-A, -B, -C, -DQB1, -DRB1, and -DPB1, all typed using DNA-based high-resolution methods. A cell population can be generated from a matched unrelated donor that is an 9/12 match for HLA-A, -B, -C, -DQB1, -DRB1, and -DPB1, all typed using DNA-based high-resolution methods.

A cell population can be generated from a matched related donor that is an 8/8 match for HLA-A, -B, -C, and -DRB1, all typed using DNA-based high-resolution methods. A cell population can be generated from a matched related donor that is an 7/8 match for HLA-A, -B, -C, and -DRB1, all typed using DNA-based high-resolution methods. A cell population can be generated from a matched related donor that is an 6/8 match for HLA-A, -B, -C, and -DRB1, all typed using DNA-based high-resolution methods. A cell population can be generated from a matched related donor that is an 10/10 match for HLA-A, -B, -C, -DQB1, and -DRB1, all typed using DNA-based high-resolution methods. A cell population can be generated from a matched related donor that is an 9/10 match for HLA-A, -B, -C, -DQB1, and -DRB1, all typed using DNA-based high-resolution methods. A cell population can be generated from a matched related donor that is an 8/10 match for HLA-A, -B, -C, -DQB1, and -DRB1, all typed using DNA-based high-resolution methods. A cell population can be generated from a matched related donor that is an 7/10 match for HLA-A, -B, -C, -DQB1, and -DRB1, all typed using DNA-based high-resolution methods. A cell population can be generated from a matched related donor that is an 12/12 match for HLA-A, -B, -C, -DQB1, -DRB1, and -DPB1, all typed using DNA-based high-resolution methods. A cell population can be generated from a matched related donor that is an 11/12 match for HLA-A, -B, -C, -DQB1, -DRB1, and -DPB1, all typed using DNA-based high-resolution methods. A cell population can be generated from a matched related donor that is an 10/12 match for HLA-A, -B, -C, -DQB1, -DRB1, and -DPB1, all typed using DNA-based high-resolution methods. A cell population can be generated from a matched related donor that is an 9/12 match for HLA-A, -B, -C, -DQB1, -DRB1, and -DPB1, all typed using DNA-based high-resolution methods.

A cell population can be generated from a matched parent donor that is an 8/8 match for HLA-A, -B, -C, and -DRB1, all typed using DNA-based high-resolution methods. A cell population can be generated from a matched parent donor that is an 7/8 match for HLA-A, -B, -C, and -DRB1, all typed using DNA-based high-resolution methods. A cell population can be generated from a matched parent donor that is an 6/8 match for HLA-A, -B, -C, and -DRB1, all typed using DNA-based high-resolution methods. A cell population can be generated from a matched parent donor that is an 10/10 match for HLA-A, -B, -C, -DQB1, and -DRB1, all typed using DNA-based high-resolution methods. A cell population can be generated from a matched parent donor that is an 9/10 match for HLA-A, -B, -C, -DQB1, and -DRB1, all typed using DNA-based high-resolution methods. A cell population can be generated from a matched parent donor that is an 8/10 match for HLA-A, -B, -C, -DQB1, and -DRB1, all typed using DNA-based high-resolution methods. A cell population can be generated from a matched parent donor that is an 7/10 match for HLA-A, -B, -C, -DQB1, and -DRB1, all typed using DNA-based high-resolution methods. A cell population can be generated from a matched parent donor that is an 12/12 match for HLA-A, -B, -C, -DQB1, -DRB1, and -DPB1, all typed using DNA-based high-resolution methods. A cell population can be generated from a matched parent donor that is an 11/12 match for HLA-A, -B, -C, -DQB1, -DRB1, and -DPB1, all typed using DNA-based high-resolution methods. A cell population can be generated from a matched parent donor that is an 10/12 match for HLA-A, -B, -C, -DQB1, -DRB1, and -DPB1, all typed using DNA-based high-resolution methods. A cell population can be generated from a matched parent donor that is an 9/12 match for HLA-A, -B, -C, -DQB1, -DRB1, and -DPB1, all typed using DNA-based high-resolution methods.

A cell population can be generated from a matched child donor that is an 8/8 match for HLA-A, -B, -C, and -DRB1, all typed using DNA-based high-resolution methods. A cell population can be generated from a matched child donor that is an 7/8 match for HLA-A, -B, -C, and -DRB1, all typed using DNA-based high-resolution methods. A cell population can be generated from a matched child donor that is an 6/8 match for HLA-A, -B, -C, and -DRB1, all typed using DNA-based high-resolution methods. A cell population can be generated from a matched child donor that is an 10/10 match for HLA-A, -B, -C, -DQB1, and -DRB1, all typed using DNA-based high-resolution methods. A cell population can be generated from a matched child donor that is an 9/10 match for HLA-A, -B, -C, -DQB1, and -DRB1, all typed using DNA-based high-resolution methods. A cell population can be generated from a matched child donor that is an 8/10 match for HLA-A, -B, -C, -DQB1, and -DRB1, all typed using DNA-based high-resolution methods. A cell population can be generated from a matched child donor that is an 7/10 match for HLA-A, -B, -C, -DQB1, and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population can be generated from a matched sibling donor that is an 8/8 match for HLA-A, -B, -C, and -DRB1, all typed using DNA-based high-resolution methods. A cell population can be generated from a matched sibling donor that is an 7/8 match for HLA-A, -B, -C, and -DRB1, all typed using DNA-based high-resolution methods. A cell population can be generated from a matched sibling donor that is an 6/8 match for HLA-A, -B, -C, and -DRB1, all typed using DNA-based high-resolution methods. A cell population can be generated from a matched sibling donor that is an 10/10 match for HLA-A, -B, -C, -DQB1 and -DRB1, all typed using DNA-based high-resolution methods. A cell population can be generated from a matched sibling donor that is an 9/10 match for HLA-A, -B, -C, -DQB1 and -DRB1, all typed using DNA-based high-resolution methods. A cell population can be generated from a matched sibling donor that is an 8/10 match for HLA-A, -B, -C, -DQB1 and -DRB1, all typed using DNA-based high-resolution methods. A cell population can be generated from a matched sibling donor that is an 7/10 match for HLA-A, -B, -C, -DQB1 and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population can be generated from a matched grandparent donor that is an 8/8 match for HLA-A, -B, -C, and -DRB1, all typed using DNA-based high-resolution methods. A cell population can be generated from a matched grandparent donor that is an 7/8 match for HLA-A, -B, -C, and -DRB1, all typed using DNA-based high-resolution methods. A cell population can be generated from a matched grandparent donor that is an 6/8 match for HLA-A, -B, -C, and -DRB1, all typed using DNA-based high-resolution methods. A cell population can be generated from a matched grandparent donor that is an 10/10 match for HLA-A, -B, -C, -DQB1, and -DRB1, all typed using DNA-based high-resolution methods. A cell population can be generated from a matched grandparent donor that is an 9/10 match for HLA-A, -B, -C, -DQB1, and -DRB1, all typed using DNA-based high-resolution methods. A cell population can be generated from a matched grandparent donor that is an 8/10 match for HLA-A, -B, -C, -DQB1, and -DRB1, all typed using DNA-based high-resolution methods. A cell population can be generated from a matched grandparent donor that is an 7/10 match for HLA-A, -B, -C, -DQB1, and -DRB1, all typed using DNA-based high-resolution methods. A cell population can be generated from a matched grandparent donor that is an 12/12 match for HLA-A, -B, -C, -DQB1, -DRB1, and -DPB1, all typed using DNA-based high-resolution methods. A cell population can be generated from a matched grandparent donor that is an 11/12 match for HLA-A, -B, -C, -DQB1, -DRB1, and -DPB1, all typed using DNA-based high-resolution methods. A cell population can be generated from a matched grandparent donor that is an 10/12 match for HLA-A, -B, -C, -DQB1, -DRB1, and -DPB1, all typed using DNA-based high-resolution methods. A cell population can be generated from a matched grandparent donor that is an 9/12 match for HLA-A, -B, -C, -DQB1, -DRB1, and -DPB1, all typed using DNA-based high-resolution methods.

A cell population can be generated from a matched grandchild donor that is an 8/8 match for HLA-A, -B, -C, and -DRB1, all typed using DNA-based high-resolution methods. A cell population can be generated from a matched grandchild donor that is an 7/8 match for HLA-A, -B, -C, and -DRB1, all typed using DNA-based high-resolution methods. A cell population can be generated from a matched grandchild donor that is an 6/8 match for HLA-A, -B, -C, and -DRB1, all typed using DNA-based high-resolution methods. A cell population can be generated from a matched grandchild donor that is an 10/10 match for HLA-A, -B, -C, -DQB1, and -DRB1, all typed using DNA-based high-resolution methods. A cell population can be generated from a matched grandchild donor that is an 9/10 match for HLA-A, -B, -C, -DQB1, and -DRB1, all typed using DNA-based high-resolution methods. A cell population can be generated from a matched grandchild donor that is an 8/10 match for HLA-A, -B, -C, -DQB1, and -DRB1, all typed using DNA-based high-resolution methods. A cell population can be generated from a matched grandchild donor that is an 7/10 match for HLA-A, -B, -C, -DQB1, and -DRB1, all typed using DNA-based high-resolution methods. A cell population can be generated from a matched grandchild donor that is an 12/12 match for HLA-A, -B, -C, -DQB1, -DRB1, and -DPB1, all typed using DNA-based high-resolution methods. A cell population can be generated from a matched grandchild donor that is an 11/12 match for HLA-A, -B, -C, -DQB1, -DRB1, and -DPB1, all typed using DNA-based high-resolution methods. A cell population can be generated from a matched grandchild donor that is an 10/12 match for HLA-A, -B, -C, -DQB1, -DRB1, and -DPB1, all typed using DNA-based high-resolution methods. A cell population can be generated from a matched grandchild donor that is an 9/12 match for HLA-A, -B, -C, -DQB1, -DRB1, and -DPB1, all typed using DNA-based high-resolution methods.

A cell population can be generated from a matched aunt donor that is an 8/8 match for HLA-A, -B, -C, and -DRB1, all typed using DNA-based high-resolution methods. A cell population can be generated from a matched aunt donor that is an 7/8 match for HLA-A, -B, -C, and -DRB1, all typed using DNA-based high-resolution methods. A cell population can be generated from a matched aunt donor that is an 6/8 match for HLA-A, -B, -C, and -DRB1, all typed using DNA-based high-resolution methods. A cell population can be generated from a matched aunt donor that is an 10/10 match for HLA-A, -B, -C, -DQB1, and -DRB1, all typed using DNA-based high-resolution methods. A cell population can be generated from a matched aunt donor that is an 9/10 match for HLA-A, -B, -C, -DQB1, and -DRB1, all typed using DNA-based high-resolution methods. A cell population can be generated from a matched aunt donor that is an 8/10 match for HLA-A, -B, -C, -DQB1, and -DRB1, all typed using DNA-based high-resolution methods. A cell population can be generated from a matched aunt donor that is an 7/10 match for HLA-A, -B, -C, -DQB1, and -DRB1, all typed using DNA-based high-resolution methods. A cell population can be generated from a matched aunt donor that is an 12/12 match for HLA-A, -B, -C, -DQB1, -DRB1, and -DPB1, all typed using DNA-based high-resolution methods. A cell population can be generated from a matched aunt donor that is an 11/12 match for HLA-A, -B, -C, -DQB1, -DRB1, and -DPB1, all typed using DNA-based high-resolution methods. A cell population can be generated from a matched aunt donor that is an 10/12 match for HLA-A, -B, -C, -DQB1, -DRB1, and -DPB1, all typed using DNA-based high-resolution methods. A cell population can be generated from a matched aunt donor that is an 9/12 match for HLA-A, -B, -C, -DQB1, -DRB1, and -DPB1, all typed using DNA-based high-resolution methods.

A cell population can be generated from a matched uncle donor that is an 8/8 match for HLA-A, -B, -C, and -DRB1, all typed using DNA-based high-resolution methods. A cell population can be generated from a matched uncle donor that is an 7/8 match for HLA-A, -B, -C, and -DRB1, all typed using DNA-based high-resolution methods. A cell population can be generated from a matched uncle donor that is an 6/8 match for HLA-A, -B, -C, and -DRB1, all typed using DNA-based high-resolution methods. A cell population can be generated from a matched uncle donor that is an 10/10 match for HLA-A, -B, -C, -DQB1, and -DRB1, all typed using DNA-based high-resolution methods. A cell population can be generated from a matched uncle donor that is an 9/10 match for HLA-A, -B, -C, -DQB1, and -DRB1, all typed using DNA-based high-resolution methods. A cell population can be generated from a matched uncle donor that is an 8/10 match for HLA-A, -B, -C, -DQB1, and -DRB1, all typed using DNA-based high-resolution methods. A cell population can be generated from a matched uncle donor that is an 7/10 match for HLA-A, -B, -C, -DQB1, and -DRB1, all typed using DNA-based high-resolution methods. A cell population can be generated from a matched uncle donor that is an 12/12 match for HLA-A, -B, -C, -DQB1, -DRB1, and -DPB1, all typed using DNA-based high-resolution methods. A cell population can be generated from a matched uncle donor that is an 11/12 match for HLA-A, -B, -C, -DQB1, -DRB1, and -DPB1, all typed using DNA-based high-resolution methods. A cell population can be generated from a matched uncle donor that is an 10/12 match for HLA-A, -B, -C, -DQB1, -DRB1, and -DPB1, all typed using DNA-based high-resolution methods. A cell population can be generated from a matched uncle donor that is an 9/12 match for HLA-A, -B, -C, -DQB1, -DRB1, and -DPB1, all typed using DNA-based high-resolution methods.

A cell population can be generated from a matched cousin donor that is an 8/8 match for HLA-A, -B, -C, and -DRB1, all typed using DNA-based high-resolution methods. A cell population can be generated from a matched cousin donor that is an 7/8 match for HLA-A, -B, -C, and -DRB1, all typed using DNA-based high-resolution methods. A cell population can be generated from a matched cousin donor that is an 6/8 match for HLA-A, -B, -C, and -DRB1, all typed using DNA-based high-resolution methods. A cell population can be generated from a matched cousin donor that is an 10/10 match for HLA-A, -B, -C, -DQB1, and -DRB1, all typed using DNA-based high-resolution methods. A cell population can be generated from a matched cousin donor that is an 9/10 match for HLA-A, -B, -C, -DQB1, and -DRB1, all typed using DNA-based high-resolution methods. A cell population can be generated from a matched cousin donor that is an 8/10 match for HLA-A, -B, -C, -DQB1, and -DRB1, all typed using DNA-based high-resolution methods. A cell population can be generated from a matched cousin donor that is an 7/10 match for HLA-A, -B, -C, -DQB1, and -DRB1, all typed using DNA-based high-resolution methods. A cell population can be generated from a matched cousin donor that is an 12/12 match for HLA-A, -B, -C, -DQB1, -DRB1, and -DPB1, all typed using DNA-based high-resolution methods. A cell population can be generated from a matched cousin donor that is an 11/12 match for HLA-A, -B, -C, -DQB1, -DRB1, and -DPB1, all typed using DNA-based high-resolution methods. A cell population can be generated from a matched cousin donor that is an 10/12 match for HLA-A, -B, -C, -DQB1, -DRB1, and -DPB1, all typed using DNA-based high-resolution methods. A cell population can be generated from a matched cousin donor that is an 9/12 match for HLA-A, -B, -C, -DQB1, -DRB1, and -DPB1, all typed using DNA-based high-resolution methods.

### 2. Specific Embodiments

In some embodiments, the HLA mismatch occurs as the result of the allogeneic related or unrelated donor being homozygous for the HLA allele while the recipient subject is heterogeneous for the HLA allele. In some embodiments, the HLA mismatch occurs as the result of the allogeneic related or unrelated donor being heterogeneous for the HLA allele while the recipient subject is homozygous for the HLA allele. In some embodiments, the HLA mismatch occurs as the result of both the allogeneic related or unrelated donor and the recipient subject being heterozygous for the HLA allele.

In various embodiments, a cell population of the present disclosure is allogeneic relative to the human subject. In some embodiments, a cell population of the present disclosure is obtained from a donor that is HLA-matched relative to the human subject. In embodiments, a cell population of the present disclosure is obtained from a donor that is HLA-mismatched relative to the human subject. In various embodiments, a cell population of the present disclosure is obtained from a donor that is haploidentical relative to the human subject.

One or more cell populations of the present disclosure can be obtained from a single donor, for example, obtained from mobilized peripheral blood apheresis of a single donor. HSPC, Treg, Tmem, iNKT, or any combination thereof can be obtained from a single donor.

One or more cell populations of the present disclosure can be obtained from one donor, and one or more additional cell populations of the present disclosure can be obtained from a second donor. One cell population of the present disclosure can be obtained from a single donor, and a second cell population of the present disclosure can be obtained from multiple donors. Cell populations of the present disclosure can be obtained from multiple donors, for example, obtained from mobilized peripheral blood apheresis of multiple donors. HSPC can be obtained from multiple donors. Treg can be obtained from multiple donors. Tmem can be obtained from multiple donors. iNKT can be obtained from multiple donors.

In embodiments, the respective cell populations are provided as separate cell populations and are derived from a single human blood donor.

A cell population can comprise cells that are from one or more donors that have each been HLA typed, for example, to determine a degree of HLA matching to a subject that will receive the cell population.

In some embodiments, the population of cells, said population of cells enriched for Treg and/or Tmem is allogeneic relative to the human subject. In some embodiments, the population of cells depleted of naïve conventional αβ-T cells or naïve conventional CD3⁺CD25⁻CD45RA⁺ T cells (Tcon) is allogeneic relative to the human subject.

In some embodiments, the population of cells enriched for Treg and/or Tmem is obtained from a donor that is HLA-matched relative to the human subject. In some embodiments, the population of cells depleted of naïve conventional αβ-T cells or naïve conventional CD3⁺CD25⁻CD45RA⁺ T cells (Tcon) is obtained from a donor that is HLA-matched relative to the human subject.

In some embodiments, the population of cells enriched for Treg and/or Tmem is obtained from a donor that is HLA-mismatched relative to the human subject. In some embodiments, the population of cells depleted of naïve conventional αβ-T cells or naïve conventional CD3⁺CD25⁻CD45RA⁺ T cells (Tcon) is obtained from a donor that is HLA-mismatched relative to the human subject.

In some embodiments, the population of cells enriched for Treg and/or Tmem is obtained from a donor that is haploidentical relative to the human subject. In some embodiments, the population of cells depleted of naïve conventional αβ-T cells or naïve conventional CD3⁺CD25⁻CD45RA⁺ T cells (Tcon) is obtained from a donor that is haploidentical relative to the human subject.

A cell population can be derived from an allogeneic donor. A cell population can be generated from a donor that is a first-degree blood relative of the subject. A cell population can be generated from a donor that is a second-degree blood relative of the subject. A cell population can be generated from a donor that is not related to the subject. A cell population can be generated from a donor that is HLA matched to a recipient subject. A cell population can be generated from a donor that is HLA mismatched to a recipient subject. A cell population can be generated from a donor that is haploidentical to a recipient subject. A cell population can be generated from a donor that is related to a recipient subject, for example, a parent, child, sibling, grandparent, grandchild, aunt, uncle, or cousin. A cell population can be generated from a donor that is at least 16 years old. A cell population can be generated from a donor that is at least 18 years old.

A cell population can be generated from a donor that meets eligibility criteria for donors of viable, leukocyte-rich cells or tissues as defined by 21 CFR § 1271 2018 and relevant FDA Guidance for Industry. For example, a cell population can be generated from a donor that meets eligibility criteria outlined in any one or more of the following: Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; and Donor Screening Recommendations to Reduce the Risk of Transmission of Zika Virus by Human Cells, Tissues, and Cellular and Tissue-Based Products, 2018). A cell population can be generated from a donor that meets any criteria for donation as specified by standard NMDP guidelines (NMDP donors).

A cell population can be generated from a donor that does not exhibit evidence of active infection. A cell population can be generated from a donor that is not seropositive for HIV-1 or -2, HTLV-1 or -2. A cell population can be generated from a donor that is not positive for anti-hepatitis C (HCV) antibody or HCV NAT. A cell population can be generated from a donor that tests negative for chronic HBV infection. A cell population can be generated from a donor that does not have high potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). A cell population can be generated from a donor that does not have signs or symptoms consistent with active Zika virus infection.

One or more cell populations of the present disclosure can be obtained from a single donor, for example, obtained from mobilized peripheral blood apheresis of a single donor. HSPC, Treg, naïve conventional αβ-T cells or naïve conventional CD3⁺CD25⁻CD45RA⁺ T cells (Tcon), iNKT, Tmem, or any combination thereof can be obtained from a single donor. Populations of cells provided in the present disclosure can be obtained from multiple donors, for example, obtained from mobilized peripheral blood apheresis of multiple donors. HSPC can be obtained from multiple donors. Treg can be obtained from multiple donors. Naïve conventional αβ-T cells or naïve conventional CD3⁺CD25⁻CD45RA⁺ T cells (Tcon) can be obtained from multiple donors. iNKT can be obtained from multiple donors. Tmem can be obtained from multiple donors. Naïve conventional αβ-T cells or naïve conventional CD3⁺CD25⁻CD45RA⁺ T cells (Tcon) can be depleted from one or more cell populations of the disclosure.

### Doses of Cells

Doses of cell populations administered to a subject (e.g., human subject) may be based on the subject's body weight. In some embodiments, a subject's body weight may be used to determine a dose of one or more cell populations to be administered to the subject. In some embodiments, a cell dose may be based on a calculated body weight for dose calculation (e.g., ideal body weight or IBW) of the subject instead of their actual weight (e.g., actual body weight). In some embodiments, IBW is a preferable method of dose calculation to avoid erroneous cell doses due to excess body fat and/or muscle mass. In some embodiments, a subject's ideal body weight can be calculated using their height and sex. In some embodiments, a subject's ideal body weight can be calculated using the method of Devine (DeVine 1974). In some embodiments, other methods that calculate a subject's IBW can be used. For instance, other methods which determine a subject's body fat percentage. In some embodiments, a dose of one or more cell populations of the present disclosure (e.g., the population of isolated CD45⁺ cells) can be based on the subject's adjusted ideal body weight (AIBW), if the subject's actual body weight is greater than 120% of the IBW.

In some embodiments a human subject of the present disclosure has a body weight that ranges from approximately 4 kilograms (kg) to approximately 200 kilograms (kg). In some embodiments, the human subject has a body weight that is approximately 4 kilograms, approximately 5 kilograms, approximately 6 kilograms, approximately 7 kilograms, approximately 8 kilograms, approximately 9 kilograms, approximately 10 kilograms, approximately 11 kilograms, approximately 12 kilograms, approximately 13 kilograms, approximately 14 kilograms, approximately 15 kilograms, approximately 16 kilograms, approximately 17 kilograms, approximately 18 kilograms, approximately 19 kilograms, approximately 20 kilograms, approximately 21 kilograms, approximately 22 kilograms, approximately 23 kilograms, approximately 24 kilograms, approximately 25 kilograms, approximately 26 kilograms, approximately 27 kilograms, approximately 28 kilograms, approximately 29 kilograms, approximately 30 kilograms, approximately 31 kilograms, approximately 32 kilograms, approximately 33 kilograms, approximately 34 kilograms, approximately 35 kilograms, approximately 36 kilograms, approximately 37 kilograms, approximately 38 kilograms, approximately 39 kilograms, approximately 40 kilograms, approximately 41 kilograms, approximately 42 kilograms, approximately 43 kilograms, approximately 44 kilograms, approximately 45 kilograms, approximately 46 kilograms, approximately 47 kilograms, approximately 48 kilograms, approximately 49 kilograms, approximately 50 kilograms, approximately 51 kilograms, approximately 52 kilograms, approximately 53 kilograms, approximately 54 kilograms, approximately 55 kilograms, approximately 56 kilograms, approximately 57 kilograms, approximately 58 kilograms, approximately 59 kilograms, approximately 60 kilograms, approximately 61 kilograms, approximately 62 kilograms, approximately 63 kilograms, approximately 64 kilograms, approximately 65 kilograms, approximately 66 kilograms, approximately 67 kilograms, approximately 68 kilograms, approximately 69 kilograms, approximately 70 kilograms, approximately 71 kilograms, approximately 72 kilograms, approximately 73 kilograms, approximately 74 kilograms, approximately 75 kilograms, approximately 76 kilograms, approximately 77 kilograms, approximately 78 kilograms, approximately 79 kilograms, approximately 80 kilograms, approximately 81 kilograms, approximately 82 kilograms, approximately 83 kilograms, approximately 84 kilograms, approximately 85 kilograms, approximately 86 kilograms, approximately 87 kilograms, approximately 88 kilograms, approximately 89 kilograms, approximately 90 kilograms, approximately 91 kilograms, approximately 92 kilograms, approximately 93 kilograms, approximately 94 kilograms, approximately 95 kilograms, approximately 96 kilograms, approximately 97 kilograms, approximately 98 kilograms, approximately 99 kilograms, approximately 100 kilograms, approximately 105 kilograms, approximately 110 kilograms, approximately 115 kilograms, approximately 120 kilograms, approximately 125 kilograms, approximately 130 kilograms, approximately 135 kilograms, approximately 140 kilograms, approximately 145 kilograms, approximately 150 kilograms, approximately 155 kilograms, approximately 160 kilograms, approximately 165 kilograms, approximately 170 kilograms, approximately 175 kilograms, approximately 180 kilograms, approximately 185 kilograms, approximately 190 kilograms, approximately 195 kilograms, or approximately 200 kilograms.

### HSPC

In some embodiments, a population of cells of the present disclosure comprising HSPC, or comprising at least one dose of HSPC, comprises from about 1×10⁴ to about 1×10⁹ HSPC per kilogram of actual body weight or ideal body weight of a human subject receiving a treatment or cellular therapy product of the present disclosure. In some embodiments, the population of cells comprising HSPC, or comprising at least one dose of HSPC, comprises from about 1×10⁴ to about 1×10⁹ HSPC per kilogram of IBW of the human subject.

A population of HSPC, or a dose of HSPC, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶ at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more HSPC per kilogram (kg) of the human subject's actual body weight or (adjusted) ideal body weight.

A population of HSPC, or a dose of HSPC, can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ HSPC per kg of the human subject's actual body weight or (adjusted) ideal body weight.

In some embodiments, a population of HSPC, or a dose of HSPC, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ HSPC per kg of the human subject's actual body weight or (adjusted) ideal body weight.

In some embodiments, a population of HSPC, or a dose of HSPC, can comprise from approximately 1.0×10⁵ to approximately 5.0×10¹⁰, from approximately 5.0×10⁵ to approximately 1.5×10¹⁰, from approximately 5.0×10⁵ to approximately 5.0×10⁸, or from approximately 1.5×10⁷ to approximately 1.5×10¹⁰ HSPC. In some embodiments, a population of HSPC, or a dose of HSPC, can comprise approximately 1.0×10⁵ or more HSPC, approximately 2.0×10⁵ or more HSPC, approximately 3.0×10⁵ or more HSPC, approximately 4.0×10⁵ or more HSPC, approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.0×10⁶ or more HSPC, approximately 1.1×10⁶ or more HSPC, approximately 1.2×10⁶ or more HSPC, approximately 1.3×10⁶ or more HSPC, approximately 1.4×10⁶ or more HSPC, approximately 1.5×10⁶ or more HSPC, approximately 1.6×10⁶ or more HSPC, approximately 1.7×10⁶ or more HSPC, approximately 1.8×10⁶ or more HSPC, approximately 1.9×10⁶ or more HSPC, approximately 2.0×10⁶ or more HSPC, approximately 2.1×10⁶ or more HSPC, approximately 2.2×10⁶ or more HSPC, approximately 2.3×10⁶ or more HSPC, approximately 2.4×10⁶ or more HSPC, approximately 2.5×10⁶ or more HSPC, approximately 2.6×10⁶ or more HSPC, approximately 2.7×10⁶ or more HSPC, approximately 2.8×10⁶ or more HSPC, approximately 2.9×10⁶ or more HSPC, approximately 3.0×10⁶ or more HSPC, approximately 3.1×10⁶ or more HSPC, approximately 3.2×10⁶ or more HSPC, approximately 3.3×10⁶ or more HSPC, approximately 3.4×10⁶ or more HSPC, approximately 3.5×10⁶ or more HSPC, approximately 3.6×10⁶ or more HSPC, approximately 3.7×10⁶ or more HSPC, approximately 3.8×10⁶ or more HSPC, approximately 3.9×10⁶ or more HSPC, approximately 4.0×10⁶ or more HSPC, approximately 4.1×10⁶ or more HSPC, approximately 4.2×10⁶ or more HSPC, approximately 4.3×10⁶ or more HSPC, approximately 4.4×10⁶ or more HSPC, approximately 4.5×10⁶ or more HSPC, approximately 4.6×10⁶ or more HSPC, approximately 4.7×10⁶ or more HSPC, approximately 4.8×10⁶ or more HSPC, approximately 4.9×10⁶ or more HSPC, approximately 5.0×10⁶ or more HSPC, approximately 5.1×10⁶ or more HSPC, approximately 5.2×10⁶ or more HSPC, approximately 5.3×10⁶ or more HSPC, approximately 5.4×10⁶ or more HSPC, approximately 5.5×10⁶ or more HSPC, approximately 5.6×10⁶ or more HSPC, approximately 5.7×10⁶ or more HSPC, approximately 5.8×10⁶ or more HSPC, approximately 5.9×10⁶ or more HSPC, approximately 6.0×10⁶ or more HSPC, approximately 6.1×10⁶ or more HSPC, approximately 6.2×10⁶ or more HSPC, approximately 6.3×10⁶ or more HSPC, approximately 6.4×10⁶ or more HSPC, approximately 6.5×10⁶ or more HSPC, approximately 6.6×10⁶ or more HSPC, approximately 6.7×10⁶ or more HSPC, approximately 6.8×10⁶ or more HSPC, approximately 6.9×10⁶ or more HSPC, approximately 7.0×10⁶ or more HSPC, approximately 7.1×10⁶ or more HSPC, approximately 7.2×10⁶ or more HSPC, approximately 7.3×10⁶ or more HSPC, approximately 7.4×10⁶ or more HSPC, approximately 7.5×10⁶ or more HSPC, approximately 7.6×10⁶ or more HSPC, approximately 7.7×10⁶ or more HSPC, approximately 7.8×10⁶ or more HSPC, approximately 7.9×10⁶ or more HSPC, approximately 8.0×10⁶ or more HSPC, approximately 8.1×10⁶ or more HSPC, approximately 8.2×10⁶ or more HSPC, approximately 8.3×10⁶ or more HSPC, approximately 8.4×10⁶ or more HSPC, approximately 8.5×10⁶ or more HSPC, approximately 8.6×10⁶ or more HSPC, approximately 8.7×10⁶ or more HSPC, approximately 8.8×10⁶ or more HSPC, approximately 8.9×10⁶ or more HSPC, approximately 9.0×10⁶ or more HSPC, approximately 9.1×10⁶ or more HSPC, approximately 9.2×10⁶ or more HSPC, approximately 9.3×10⁶ or more HSPC, approximately 9.4×10⁶ or more HSPC, approximately 9.5×10⁶ or more HSPC, approximately 9.6×10⁶ or more HSPC, approximately 9.7×10⁶ or more HSPC, approximately 9.8×10⁶ or more HSPC, approximately 9.9×10⁶ or more HSPC, approximately 1.0×10⁷ or more HSPC, approximately 1.1×10⁷ or more HSPC, approximately 1.2×10⁷ or more HSPC, approximately 1.3×10⁷ or more HSPC, approximately 1.4×10⁷ or more HSPC, approximately 1.5×10⁷ or more HSPC, approximately 1.6×10⁷ or more HSPC, approximately 1.7×10⁷ or more HSPC, approximately 1.8×10⁷ or more HSPC, approximately 1.9×10⁷ or more HSPC, approximately 2.0×10⁷ or more HSPC, approximately 2.1×10⁷ or more HSPC, approximately 2.2×10⁷ or more HSPC, approximately 2.3×10⁷ or more HSPC, approximately 2.4×10⁷ or more HSPC, approximately 2.5×10⁷ or more HSPC, approximately 2.6×10⁷ or more HSPC, approximately 2.7×10⁷ or more HSPC, approximately 2.8×10⁷ or more HSPC, approximately 2.9×10⁷ or more HSPC, approximately 3.0×10⁷ or more HSPC, approximately 3.1×10⁷ or more HSPC, approximately 3.2×10⁷ or more HSPC, approximately 3.3×10⁷ or more HSPC, approximately 3.4×10⁷ or more HSPC, approximately 3.5×10⁷ or more HSPC, approximately 3.6×10⁷ or more HSPC, approximately 3.7×10⁷ or more HSPC, approximately 3.8×10⁷ or more HSPC, approximately 3.9×10⁷ or more HSPC, approximately 4.0×10⁷ or more HSPC, approximately 4.1×10⁷ or more HSPC, approximately 4.2×10⁷ or more HSPC, approximately 4.3×10⁷ or more HSPC, approximately 4.4×10⁷ or more HSPC, approximately 4.5×10⁷ or more HSPC, approximately 4.6×10⁷ or more HSPC, approximately 4.7×10⁷ or more HSPC, approximately 4.8×10⁷ or more HSPC, approximately 4.9×10⁷ or more HSPC, approximately 5.0×10⁷ or more HSPC, approximately 5.1×10⁷ or more HSPC, approximately 5.2×10⁷ or more HSPC, approximately 5.3×10⁷ or more HSPC, approximately 5.4×10⁷ or more HSPC, approximately 5.5×10⁷ or more HSPC, approximately 5.6×10⁷ or more HSPC, approximately 5.7×10⁷ or more HSPC, approximately 5.8×10⁷ or more HSPC, approximately 5.9×10⁷ or more HSPC, approximately 6.0×10⁷ or more HSPC, approximately 6.1×10⁷ or more HSPC, approximately 6.2×10⁷ or more HSPC, approximately 6.3×10⁷ or more HSPC, approximately 6.4×10⁷ or more HSPC, approximately 6.5×10⁷ or more HSPC, approximately 6.6×10⁷ or more HSPC, approximately 6.7×10⁷ or more HSPC, approximately 6.8×10⁷ or more HSPC, approximately 6.9×10⁷ or more HSPC, approximately 7.0×10⁷ or more HSPC, approximately 7.1×10⁷ or more HSPC, approximately 7.2×10⁷ or more HSPC, approximately 7.3×10⁷ or more HSPC, approximately 7.4×10⁷ or more HSPC, approximately 7.5×10⁷ or more HSPC, approximately 7.6×10⁷ or more HSPC, approximately 7.7×10⁷ or more HSPC, approximately 7.8×10⁷ or more HSPC, approximately 7.9×10⁷ or more HSPC, approximately 8.0×10⁷ or more HSPC, approximately 8.1×10⁷ or more HSPC, approximately 8.2×10⁷ or more HSPC, approximately 8.3×10⁷ or more HSPC, approximately 8.4×10⁷ or more HSPC, approximately 8.5×10⁷ or more HSPC, approximately 8.6×10⁷ or more HSPC, approximately 8.7×10⁷ or more HSPC, approximately 8.8×10⁷ or more HSPC, approximately 8.9×10⁷ or more HSPC, approximately 9.0×10⁷ or more HSPC, approximately 9.1×10⁷ or more HSPC, approximately 9.2×10⁷ or more HSPC, approximately 9.3×10⁷ or more HSPC, approximately 9.4×10⁷ or more HSPC, approximately 9.5×10⁷ or more HSPC, approximately 9.6×10⁷ or more HSPC, approximately 9.7×10⁷ or more HSPC, approximately 9.8×10⁷ or more HSPC, approximately 9.9×10⁷ or more HSPC, approximately 1.0×10⁸ or more HSPC, approximately 1.1×10⁸ or more HSPC, approximately 1.2×10⁸ or more HSPC, approximately 1.3×10⁸ or more HSPC, approximately 1.4×10⁸ or more HSPC, approximately 1.5×10⁸ or more HSPC, approximately 1.6×10⁸ or more HSPC, approximately 1.7×10⁸ or more HSPC, approximately 1.8×10⁸ or more HSPC, approximately 1.9×10⁸ or more HSPC, approximately 2.0×10⁸ or more HSPC, approximately 2.1×10⁸ or more HSPC, approximately 2.2×10⁸ or more HSPC, approximately 2.3×10⁸ or more HSPC, approximately 2.4×10⁸ or more HSPC, approximately 2.5×10⁸ or more HSPC, approximately 2.6×10⁸ or more HSPC, approximately 2.7×10⁸ or more HSPC, approximately 2.8×10⁸ or more HSPC, approximately 2.9×10⁸ or more HSPC, approximately 3.0×10⁸ or more HSPC, approximately 3.1×10⁸ or more HSPC, approximately 3.2×10⁸ or more HSPC, approximately 3.3×10⁸ or more HSPC, approximately 3.4×10⁸ or more HSPC, approximately 3.5×10⁸ or more HSPC, approximately 3.6×10⁸ or more HSPC, approximately 3.7×10⁸ or more HSPC, approximately 3.8×10⁸ or more HSPC, approximately 3.9×10⁸ or more HSPC, approximately 4.0×10⁸ or more HSPC, approximately 4.1×10⁸ or more HSPC, approximately 4.2×10⁸ or more HSPC, approximately 4.3×10⁸ or more HSPC, approximately 4.4×10⁸ or more HSPC, approximately 4.5×10⁸ or more HSPC, approximately 4.6×10⁸ or more HSPC, approximately 4.7×10⁸ or more HSPC, approximately 4.8×10⁸ or more HSPC, approximately 4.9×10⁸ or more HSPC, approximately 5.0×10⁸ or more HSPC, approximately 5.1×10⁸ or more HSPC, approximately 5.2×10⁸ or more HSPC, approximately 5.3×10⁸ or more HSPC, approximately 5.4×10⁸ or more HSPC, approximately 5.5×10⁸ or more HSPC, approximately 5.6×10⁸ or more HSPC, approximately 5.7×10⁸ or more HSPC, approximately 5.8×10⁸ or more HSPC, approximately 5.9×10⁸ or more HSPC, approximately 6.0×10⁸ or more HSPC, approximately 6.1×10⁸ or more HSPC, approximately 6.2×10⁸ or more HSPC, approximately 6.3×10⁸ or more HSPC, approximately 6.4×10⁸ or more HSPC, approximately 6.5×10⁸ or more HSPC, approximately 6.6×10⁸ or more HSPC, approximately 6.7×10⁸ or more HSPC, approximately 6.8×10⁸ or more HSPC, approximately 6.9×10⁸ or more HSPC, approximately 7.0×10⁸ or more HSPC, approximately 7.1×10⁸ or more HSPC, approximately 7.2×10⁸ or more HSPC, approximately 7.3×10⁸ or more HSPC, approximately 7.4×10⁸ or more HSPC, approximately 7.5×10⁸ or more HSPC, approximately 7.6×10⁸ or more HSPC, approximately 7.7×10⁸ or more HSPC, approximately 7.8×10⁸ or more HSPC, approximately 7.9×10⁸ or more HSPC, approximately 8.0×10⁸ or more HSPC, approximately 8.1×10⁸ or more HSPC, approximately 8.2×10⁸ or more HSPC, approximately 8.3×10⁸ or more HSPC, approximately 8.4×10⁸ or more HSPC, approximately 8.5×10⁸ or more HSPC, approximately 8.6×10⁸ or more HSPC, approximately 8.7×10⁸ or more HSPC, approximately 8.8×10⁸ or more HSPC, approximately 8.9×10⁸ or more HSPC, approximately 9.0×10⁸ or more HSPC, approximately 9.1×10⁸ or more HSPC, approximately 9.2×10⁸ or more HSPC, approximately 9.3×10⁸ or more HSPC, approximately 9.4×10⁸ or more HSPC, approximately 9.5×10⁸ or more HSPC, approximately 9.6×10⁸ or more HSPC, approximately 9.7×10⁸ or more HSPC, approximately 9.8×10⁸ or more HSPC, approximately 9.9×10⁸ or more HSPC, approximately 1.0×10⁹ or more HSPC, approximately 1.1×10⁹ or more HSPC, approximately 1.2×10⁹ or more HSPC, approximately 1.3×10⁹ or more HSPC, approximately 1.4×10⁹ or more HSPC, approximately 1.5×10⁹ or more HSPC, approximately 1.6×10⁹ or more HSPC, approximately 1.7×10⁹ or more HSPC, approximately 1.8×10⁹ or more HSPC, approximately 1.9×10⁹ or more HSPC, approximately 2.0×10⁹ or more HSPC, approximately 2.1×10⁹ or more HSPC, approximately 2.2×10⁹ or more HSPC, approximately 2.3×10⁹ or more HSPC, approximately 2.4×10⁹ or more HSPC, approximately 2.5×10⁹ or more HSPC, approximately 2.6×10⁹ or more HSPC, approximately 2.7×10⁹ or more HSPC, approximately 2.8×10⁹ or more HSPC, approximately 2.9×10⁹ or more HSPC, approximately 3.0×10⁹ or more HSPC, approximately 3.1×10⁹ or more HSPC, approximately 3.2×10⁹ or more HSPC, approximately 3.3×10⁹ or more HSPC, approximately 3.4×10⁹ or more HSPC, approximately 3.5×10⁹ or more HSPC, approximately 3.6×10⁹ or more HSPC, approximately 3.7×10⁹ or more HSPC, approximately 3.8×10⁹ or more HSPC, approximately 3.9×10⁹ or more HSPC, approximately 4.0×10⁹ or more HSPC, approximately 4.1×10⁹ or more HSPC, approximately 4.2×10⁹ or more HSPC, approximately 4.3×10⁹ or more HSPC, approximately 4.4×10⁹ or more HSPC, approximately 4.5×10⁹ or more HSPC, approximately 4.6×10⁹ or more HSPC, approximately 4.7×10⁹ or more HSPC, approximately 4.8×10⁹ or more HSPC, approximately 4.9×10⁹ or more HSPC, approximately 5.0×10⁹ or more HSPC, approximately 5.1×10⁹ or more HSPC, approximately 5.2×10⁹ or more HSPC, approximately 5.3×10⁹ or more HSPC, approximately 5.4×10⁹ or more HSPC, approximately 5.5×10⁹ or more HSPC, approximately 5.6×10⁹ or more HSPC, approximately 5.7×10⁹ or more HSPC, approximately 5.8×10⁹ or more HSPC, approximately 5.9×10⁹ or more HSPC, approximately 6.0×10⁹ or more HSPC, approximately 6.1×10⁹ or more HSPC, approximately 6.2×10⁹ or more HSPC, approximately 6.3×10⁹ or more HSPC, approximately 6.4×10⁹ or more HSPC, approximately 6.5×10⁹ or more HSPC, approximately 6.6×10⁹ or more HSPC, approximately 6.7×10⁹ or more HSPC, approximately 6.8×10⁹ or more HSPC, approximately 6.9×10⁹ or more HSPC, approximately 7.0×10⁹ or more HSPC, approximately 7.1×10⁹ or more HSPC, approximately 7.2×10⁹ or more HSPC, approximately 7.3×10⁹ or more HSPC, approximately 7.4×10⁹ or more HSPC, approximately 7.5×10⁹ or more HSPC, approximately 7.6×10⁹ or more HSPC, approximately 7.7×10⁹ or more HSPC, approximately 7.8×10⁹ or more HSPC, approximately 7.9×10⁹ or more HSPC, approximately 8.0×10⁹ or more HSPC, approximately 8.1×10⁹ or more HSPC, approximately 8.2×10⁹ or more HSPC, approximately 8.3×10⁹ or more HSPC, approximately 8.4×10⁹ or more HSPC, approximately 8.5×10⁹ or more HSPC, approximately 8.6×10⁹ or more HSPC, approximately 8.7×10⁹ or more HSPC, approximately 8.8×10⁹ or more HSPC, approximately 8.9×10⁹ or more HSPC, approximately 9.0×10⁹ or more HSPC, approximately 9.1×10⁹ or more HSPC, approximately 9.2×10⁹ or more HSPC, approximately 9.3×10⁹ or more HSPC, approximately 9.4×10⁹ or more HSPC, approximately 9.5×10⁹ or more HSPC, approximately 9.6×10⁹ or more HSPC, approximately 9.7×10⁹ or more HSPC, approximately 9.8×10⁹ or more HSPC, approximately 9.9×10⁹ or more HSPC, approximately 1.0×10¹⁰ or more HSPC, approximately 1.1×10¹⁰ or more HSPC, approximately 1.2×10¹⁰ or more HSPC, approximately 1.3×10¹⁰ or more HSPC, approximately 1.4×10¹⁰ or more HSPC, approximately 1.5×10¹⁰ or more HSPC, approximately 1.6×10¹⁰ or more HSPC, approximately 1.7×10¹⁰ or more HSPC, approximately 1.8×10¹⁰ or more HSPC, approximately 1.9×10¹⁰ or more HSPC, approximately 2.0×10¹⁰ or more HSPC, approximately 2.1×10¹⁰ or more HSPC, approximately 2.2×10¹⁰ or more HSPC, approximately 2.3×10¹⁰ or more HSPC, approximately 2.4×10¹⁰ or more HSPC, approximately 2.5×10¹⁰ or more HSPC, approximately 2.6×10¹⁰ or more HSPC, approximately 2.7×10¹⁰ or more HSPC, approximately 2.8×10¹⁰ or more HSPC, approximately 2.9×10¹⁰ or more HSPC, approximately 3.0×10¹⁰ or more HSPC, approximately 3.1×10¹⁰ or more HSPC, approximately 3.2×10¹⁰ or more HSPC, approximately 3.3×10¹⁰ or more HSPC, approximately 3.4×10¹⁰ or more HSPC, approximately 3.5×10¹⁰ or more HSPC, approximately 3.6×10¹⁰ or more HSPC, approximately 3.7×10¹⁰ or more HSPC, approximately 3.8×10¹⁰ or more HSPC, approximately 3.9×10¹⁰ or more HSPC, approximately 4.0×10¹⁰ or more HSPC, approximately 4.1×10¹⁰ or more HSPC, approximately 4.2×10¹⁰ or more HSPC, approximately 4.3×10¹⁰ or more HSPC, approximately 4.4×10¹⁰ or more HSPC, approximately 4.5×10¹⁰ or more HSPC, approximately 4.6×10¹⁰ or more HSPC, approximately 4.7×10¹⁰ or more HSPC, approximately 4.8×110¹⁰ or more HSPC, approximately 4.9×10¹⁰ or more HSPC, or approximately 5.0×10¹⁰ or more HSPC.

A population of HSPC, or a dose of HSPC, can have a defined level of purity for CD34⁺ cells. For example, a population of isolated CD45⁺ cells comprising HSPC, or a dose of HSPC, can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more CD34⁺ cells as a percentage of total cells, as a percentage of nucleated cells, or as a percentage of CD45⁺ cells in a population of isolate CD45⁺ cells.

In embodiments, at least one of the cell populations of the present disclosure comprises less than about 5 EU of endotoxins/ml of respective suspension liquid.

A population of HSPC, or a dose of HSPC, can comprise 0.5 EU/ml endotoxins to 10 EU/ml endotoxins. A population of HSPC, or a dose of HSPC, can comprise at least 0.5 EU/ml endotoxins. A population of CD34+ cells (e.g., HSPC) can comprise at most 10 EU/ml endotoxins. A population of HSPC, or a dose of HSPC, can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A population of CD34+ cells (e.g., HSPC) can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of HSPC, or a dose of HSPC, can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of CD34+ cells (e.g., HSPC) can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A population of HSPC, or a dose of HSPC, can comprise 0.5% w/w to 10% w/w unbound reagents. These unbound reagents may include any affinity reagents used for the sorting of HSPC, for instance, antibodies, or purification particles or magnetic particles. A population of HSPC, or a dose of HSPC, can comprise at least 0.5% w/w unbound reagents. A population of HSPC, or a dose of HSPC, can comprise at most 10% w/w unbound reagents. A population of HSPC, or a dose of HSPC, can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A population of HSPC, or a dose of HSPC, can comprise 10% w/w, 8% w/w, 6% w/w, 5% w/w, 4% w/w, 2% w/w, 1% w/w, or 0.5% w/w unbound reagents. A population of HSPC, or a dose of HSPC, can comprise at least 8% w/w, 6% w/w, 5% w/w, 4% w/w, 2% w/w, 1% w/w, or 0.5% w/w unbound reagents. A population of HSPC, or a dose of HSPC, can comprise at most 10% w/w, 8% w/w, 6% w/w, 5% w/w, 4% w/w, 2% w/w or 1% w/w unbound reagents.

A population of HSPC, or a dose of HSPC, can comprise 5×10³ to 90×10³ microbeads per cell. These microbeads may comprise microbeads used to purify the HSPC population, for instance, an anti-CD34 antibody comprising microbead used to sort the HSPC population. A population of HSPC, or a dose of HSPC, can comprise at least 5×10³ microbeads per cell. A population of HSPC, or a dose of HSPC, can comprise at most 90×10³ microbeads per cell. A population of HSPC, or a dose of HSPC, can comprise 90×10³ to 70×10³, 90×10³ to 50×10³, 90×10³ to 40×10³, 90×10³ to 30×10³, 90×10³ to 20×10³, 90×10³ to 10×10³, 90×10³ to 5×10³, 70×10³ to 50×10³, 70×10³ to 40×10³, 70×10³ to 30×10³, 70×10³ to 20×10³, 70×10³ to 10×10³, 70×10³ to 5×10³, 50×10³ to 40×10³, 50×10³ to 30×10³, 50×10³ to 20×10³, 50×10³ to 10×10³, 50×10³ to 5×10³, 40×10³ to 30×10³, 40×10³ to 20×10³, 40×10³ to 10×10³, 40×10³ to 5×10³, 30×10³ to 20×10³, 30×10³ to 10×10³, 30×10³ to 5×10³, 20×10³ to 10×10³, 20×10³ to 5×10³, or 10×10³ to 5×10³ microbeads per cell. A population of HSPC, or a dose of HSPC, can comprise 90×10³, 70×10³, 50×10³, 40×10³, 30×10³, 20×10³, 10×10³, or 5×10³ microbeads per cell. A population of HSPC, or a dose of HSPC, can comprise at least 70×10³, 50×10³, 40×10³, 30×10³, 20×10³, 10×10³, or 5×10³ microbeads per cell. A population of HSPC, or a dose of HSPC, can comprise at most 90×10³, 70×10³, 50×10³, 40×10³, 30×10³, 20×10³, or 10×10³ microbeads per cell.

### Treg

In some embodiments, the population of cells of the present disclosure enriched for Treg, or comprising at least one dose of Treg, comprises from about 1×10⁴ to about 1×10⁹ Treg per kilogram of actual body weight or ideal body weight of a human subject receiving a treatment or cellular therapy product of the present disclosure. In some embodiments, the population of cells enriched for Treg, or comprising at least one dose of Treg, comprises from about 1×10⁴ to about 1×10⁹ Treg per kilogram of IBW of the human subject.

A population of cells enriched for Treg, or comprising at least one dose of Treg, can comprise at least about 1×10⁴, at least about 1×10⁵, at least about 2×10⁴, at least about 3×10⁴, at least about 4×10⁴, at least about 5×10⁴, at least about 6×10⁴, at least about 7×10⁴, at least about 8×10⁴, least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the cell population enriched for Treg and/or Treg per kg of the human subject's actual body weight or (adjusted) ideal body weight (e.g., where Treg are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells enriched for Treg, or comprising at least one dose of Treg, can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the cell population enriched for Treg and/or Treg per kg of the human subject's actual body weight or (adjusted) ideal body weight (e.g., where Treg are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

For example, a population of cells enriched for Treg, or comprising at least one dose of Treg, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the cell population enriched for Treg and/or Treg per kg of the human subject's actual body weight or (adjusted) ideal body weight (e.g., where Treg are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

In some embodiments, the population of cells enriched for Treg, or comprising at least one dose of Treg, comprises from approximately 1.0×10⁵ to approximately 5.0×10⁹, from approximately 5.0×10⁵ to approximately 3.0×10⁹, from approximately 1.5×10⁷ to approximately 3.0×10⁹, or from approximately 5.0×10⁵ to approximately 1.0×10⁸ Treg, e.g., fresh isolated Treg in the population of cells enriched for Treg. In some embodiments, the population of cells enriched for Treg or comprising at least one dose of Treg, comprises approximately 1.0×10⁵ or more Treg, approximately 2.0×10⁵ or more Treg, approximately 3.0×10⁵ or more Treg, approximately 4.0×10⁵ or more Treg, approximately 5.0×10⁵ or more Treg, approximately 6.0×10⁵ or more Treg, approximately 7.0×10⁵ or more Treg, approximately 8.0×10⁵ or more Treg, approximately 9.0×10⁵ or more Treg, approximately 1.0×10⁶ or more Treg, approximately 1.0×10⁶ or more Treg, approximately 1.1×10⁶ or more Treg, approximately 1.2×10⁶ or more Treg, approximately 1.3×10⁶ or more Treg, approximately 1.4×10⁶ or more Treg, approximately 1.5×10⁶ or more Treg, approximately 1.6×10⁶ or more Treg, approximately 1.7×10⁶ or more Treg, approximately 1.8×10⁶ or more Treg, approximately 1.9×10⁶ or more Treg, approximately 2.0×10⁶ or more Treg, approximately 2.1×10⁶ or more Treg, approximately 2.2×10⁶ or more Treg, approximately 2.3×10⁶ or more Treg, approximately 2.4×10⁶ or more Treg, approximately 2.5×10⁶ or more Treg, approximately 2.6×10⁶ or more Treg, approximately 2.7×10⁶ or more Treg, approximately 2.8×10⁶ or more Treg, approximately 2.9×10⁶ or more Treg, approximately 3.0×10⁶ or more Treg, approximately 3.1×10⁶ or more Treg, approximately 3.2×10⁶ or more Treg, approximately 3.3×10⁶ or more Treg, approximately 3.4×10⁶ or more Treg, approximately 3.5×10⁶ or more Treg, approximately 3.6×10⁶ or more Treg, approximately 3.7×10⁶ or more Treg, approximately 3.8×10⁶ or more Treg, approximately 3.9×10⁶ or more Treg, approximately 4.0×10⁶ or more Treg, approximately 4.1×10⁶ or more Treg, approximately 4.2×10⁶ or more Treg, approximately 4.3×10⁶ or more Treg, approximately 4.4×10⁶ or more Treg, approximately 4.5×10⁶ or more Treg, approximately 4.6×10⁶ or more Treg, approximately 4.7×10⁶ or more Treg, approximately 4.8×10⁶ or more Treg, approximately 4.9×10⁶ or more Treg, approximately 5.0×10⁶ or more Treg, approximately 5.1×10⁶ or more Treg, approximately 5.2×10⁶ or more Treg, approximately 5.3×10⁶ or more Treg, approximately 5.4×10⁶ or more Treg, approximately 5.5×10⁶ or more Treg, approximately 5.6×10⁶ or more Treg, approximately 5.7×10⁶ or more Treg, approximately 5.8×10⁶ or more Treg, approximately 5.9×10⁶ or more Treg, approximately 6.0×10⁶ or more Treg, approximately 6.1×10⁶ or more Treg, approximately 6.2×10⁶ or more Treg, approximately 6.3×10⁶ or more Treg, approximately 6.4×10⁶ or more Treg, approximately 6.5×10⁶ or more Treg, approximately 6.6×10⁶ or more Treg, approximately 6.7×10⁶ or more Treg, approximately 6.8×10⁶ or more Treg, approximately 6.9×10⁶ or more Treg, approximately 7.0×10⁶ or more Treg, approximately 7.1×10⁶ or more Treg, approximately 7.2×10⁶ or more Treg, approximately 7.3×10⁶ or more Treg, approximately 7.4×10⁶ or more Treg, approximately 7.5×10⁶ or more Treg, approximately 7.6×10⁶ or more Treg, approximately 7.7×10⁶ or more Treg, approximately 7.8×10⁶ or more Treg, approximately 7.9×10⁶ or more Treg, approximately 8.0×10⁶ or more Treg, approximately 8.1×10⁶ or more Treg, approximately 8.2×10⁶ or more Treg, approximately 8.3×10⁶ or more Treg, approximately 8.4×10⁶ or more Treg, approximately 8.5×10⁶ or more Treg, approximately 8.6×10⁶ or more Treg, approximately 8.7×10⁶ or more Treg, approximately 8.8×10⁶ or more Treg, approximately 8.9×10⁶ or more Treg, approximately 9.0×10⁶ or more Treg, approximately 9.1×10⁶ or more Treg, approximately 9.2×10⁶ or more Treg, approximately 9.3×10⁶ or more Treg, approximately 9.4×10⁶ or more Treg, approximately 9.5×10⁶ or more Treg, approximately 9.6×10⁶ or more Treg, approximately 9.7×10⁶ or more Treg, approximately 9.8×10⁶ or more Treg, approximately 9.9×10⁶ or more Treg, approximately 1.0×10⁷ or more Treg, approximately 1.1×10⁷ or more Treg, approximately 1.2×10⁷ or more Treg, approximately 1.3×10⁷ or more Treg, approximately 1.4×10⁷ or more Treg, approximately 1.5×10⁷ or more Treg, approximately 1.6×10⁷ or more Treg, approximately 1.7×10⁷ or more Treg, approximately 1.8×10⁷ or more Treg, approximately 1.9×10⁷ or more Treg, approximately 2.0×10⁷ or more Treg, approximately 2.1×10⁷ or more Treg, approximately 2.2×10⁷ or more Treg, approximately 2.3×10⁷ or more Treg, approximately 2.4×10⁷ or more Treg, approximately 2.5×10⁷ or more Treg, approximately 2.6×10⁷ or more Treg, approximately 2.7×10⁷ or more Treg, approximately 2.8×10⁷ or more Treg, approximately 2.9×10⁷ or more Treg, approximately 3.0×10⁷ or more Treg, approximately 3.1×10⁷ or more Treg, approximately 3.2×10⁷ or more Treg, approximately 3.3×10⁷ or more Treg, approximately 3.4×10⁷ or more Treg, approximately 3.5×10⁷ or more Treg, approximately 3.6×10⁷ or more Treg, approximately 3.7×10⁷ or more Treg, approximately 3.8×10⁷ or more Treg, approximately 3.9×10⁷ or more Treg, approximately 4.0×10⁷ or more Treg, approximately 4.1×10⁷ or more Treg, approximately 4.2×10⁷ or more Treg, approximately 4.3×10⁷ or more Treg, approximately 4.4×10⁷ or more Treg, approximately 4.5×10⁷ or more Treg, approximately 4.6×10⁷ or more Treg, approximately 4.7×10⁷ or more Treg, approximately 4.8×10⁷ or more Treg, approximately 4.9×10⁷ or more Treg, approximately 5.0×10⁷ or more Treg, approximately 5.1×10⁷ or more Treg, approximately 5.2×10⁷ or more Treg, approximately 5.3×10⁷ or more Treg, approximately 5.4×10⁷ or more Treg, approximately 5.5×10⁷ or more Treg, approximately 5.6×10⁷ or more Treg, approximately 5.7×10⁷ or more Treg, approximately 5.8×10⁷ or more Treg, approximately 5.9×10⁷ or more Treg, approximately 6.0×10⁷ or more Treg, approximately 6.1×10⁷ or more Treg, approximately 6.2×10⁷ or more Treg, approximately 6.3×10⁷ or more Treg, approximately 6.4×10⁷ or more Treg, approximately 6.5×10⁷ or more Treg, approximately 6.6×10⁷ or more Treg, approximately 6.7×10⁷ or more Treg, approximately 6.8×10⁷ or more Treg, approximately 6.9×10⁷ or more Treg, approximately 7.0×10⁷ or more Treg, approximately 7.1×10⁷ or more Treg, approximately 7.2×10⁷ or more Treg, approximately 7.3×10⁷ or more Treg, approximately 7.4×10⁷ or more Treg, approximately 7.5×10⁷ or more Treg, approximately 7.6×10⁷ or more Treg, approximately 7.7×10⁷ or more Treg, approximately 7.8×10⁷ or more Treg, approximately 7.9×10⁷ or more Treg, approximately 8.0×10⁷ or more Treg, approximately 8.1×10⁷ or more Treg, approximately 8.2×10⁷ or more Treg, approximately 8.3×10⁷ or more Treg, approximately 8.4×10⁷ or more Treg, approximately 8.5×10⁷ or more Treg, approximately 8.6×10⁷ or more Treg, approximately 8.7×10⁷ or more Treg, approximately 8.8×10⁷ or more Treg, approximately 8.9×10⁷ or more Treg, approximately 9.0×10⁷ or more Treg, approximately 9.1×10⁷ or more Treg, approximately 9.2×10⁷ or more Treg, approximately 9.3×10⁷ or more Treg, approximately 9.4×10⁷ or more Treg, approximately 9.5×10⁷ or more Treg, approximately 9.6×10⁷ or more Treg, approximately 9.7×10⁷ or more Treg, approximately 9.8×10⁷ or more Treg, approximately 9.9×10⁷ or more Treg, approximately 1.0×10⁸ or more Treg, approximately 1.1×10⁸ or more Treg, approximately 1.2×10⁸ or more Treg, approximately 1.3×10⁸ or more Treg, approximately 1.4×10⁸ or more Treg, approximately 1.5×10⁸ or more Treg, approximately 1.6×10⁸ or more Treg, approximately 1.7×10⁸ or more Treg, approximately 1.8×10⁸ or more Treg, approximately 1.9×10⁸ or more Treg, approximately 2.0×10⁸ or more Treg, approximately 2.1×10⁸ or more Treg, approximately 2.2×10⁸ or more Treg, approximately 2.3×10⁸ or more Treg, approximately 2.4×10⁸ or more Treg, approximately 2.5×10⁸ or more Treg, approximately 2.6×10⁸ or more Treg, approximately 2.7×10⁸ or more Treg, approximately 2.8×10⁸ or more Treg, approximately 2.9×10⁸ or more Treg, approximately 3.0×10⁸ or more Treg, approximately 3.1×10⁸ or more Treg, approximately 3.2×10⁸ or more Treg, approximately 3.3×10⁸ or more Treg, approximately 3.4×10⁸ or more Treg, approximately 3.5×10⁸ or more Treg, approximately 3.6×10⁸ or more Treg, approximately 3.7×10⁸ or more Treg, approximately 3.8×10⁸ or more Treg, approximately 3.9×10⁸ or more Treg, approximately 4.0×10⁸ or more Treg, approximately 4.1×10⁸ or more Treg, approximately 4.2×10⁸ or more Treg, approximately 4.3×10⁸ or more Treg, approximately 4.4×10⁸ or more Treg, approximately 4.5×10⁸ or more Treg, approximately 4.6×10⁸ or more Treg, approximately 4.7×10⁸ or more Treg, approximately 4.8×10⁸ or more Treg, approximately 4.9×10⁸ or more Treg, approximately 5.0×10⁸ or more Treg, approximately 5.1×10⁸ or more Treg, approximately 5.2×10⁸ or more Treg, approximately 5.3×10⁸ or more Treg, approximately 5.4×10⁸ or more Treg, approximately 5.5×10⁸ or more Treg, approximately 5.6×10⁸ or more Treg, approximately 5.7×10⁸ or more Treg, approximately 5.8×10⁸ or more Treg, approximately 5.9×10⁸ or more Treg, approximately 6.0×10⁸ or more Treg, approximately 6.1×10⁸ or more Treg, approximately 6.2×10⁸ or more Treg, approximately 6.3×10⁸ or more Treg, approximately 6.4×10⁸ or more Treg, approximately 6.5×10⁸ or more Treg, approximately 6.6×10⁸ or more Treg, approximately 6.7×10⁸ or more Treg, approximately 6.8×10⁸ or more Treg, approximately 6.9×10⁸ or more Treg, approximately 7.0×10⁸ or more Treg, approximately 7.1×10⁸ or more Treg, approximately 7.2×10⁸ or more Treg, approximately 7.3×10⁸ or more Treg, approximately 7.4×10⁸ or more Treg, approximately 7.5×10⁸ or more Treg, approximately 7.6×10⁸ or more Treg, approximately 7.7×10⁸ or more Treg, approximately 7.8×10⁸ or more Treg, approximately 7.9×10⁸ or more Treg, approximately 8.0×10⁸ or more Treg, approximately 8.1×10⁸ or more Treg, approximately 8.2×10⁸ or more Treg, approximately 8.3×10⁸ or more Treg, approximately 8.4×10⁸ or more Treg, approximately 8.5×10⁸ or more Treg, approximately 8.6×10⁸ or more Treg, approximately 8.7×10⁸ or more Treg, approximately 8.8×10⁸ or more Treg, approximately 8.9×10⁸ or more Treg, approximately 9.0×10⁸ or more Treg, approximately 9.1×10⁸ or more Treg, approximately 9.2×10⁸ or more Treg, approximately 9.3×10⁸ or more Treg, approximately 9.4×10⁸ or more Treg, approximately 9.5×10⁸ or more Treg, approximately 9.6×10⁸ or more Treg, approximately 9.7×10⁸ or more Treg, approximately 9.8×10⁸ or more Treg, approximately 9.9×10⁸ or more Treg, approximately 1.0×10⁹ or more Treg, approximately 1.1×10⁹ or more Treg, approximately 1.2×10⁹ or more Treg, approximately 1.3×10⁹ or more Treg, approximately 1.4×10⁹ or more Treg, approximately 1.5×10⁹ or more Treg, approximately 1.6×10⁹ or more Treg, approximately 1.7×10⁹ or more Treg, approximately 1.8×10⁹ or more Treg, approximately 1.9×10⁹ or more Treg, approximately 2.0×10⁹ or more Treg, approximately 2.1×10⁹ or more Treg, approximately 2.2×10⁹ or more Treg, approximately 2.3×10⁹ or more Treg, approximately 2.4×10⁹ or more Treg, approximately 2.5×10⁹ or more Treg, approximately 2.6×10⁹ or more Treg, approximately 2.7×10⁹ or more Treg, approximately 2.8×10⁹ or more Treg, approximately 2.9×10⁹ or more Treg, approximately 3.0×10⁹ or more Treg, approximately 3.1×10⁹ or more Treg, approximately 3.2×10⁹ or more Treg, approximately 3.3×10⁹ or more Treg, approximately 3.4×10⁹ or more Treg, approximately 3.5×10⁹ or more Treg, approximately 3.6×10⁹ or more Treg, approximately 3.7×10⁹ or more Treg, approximately 3.8×10⁹ or more Treg, approximately 3.9×10⁹ or more Treg, approximately 4.0×10⁹ or more Treg, approximately 4.1×10⁹ or more Treg, approximately 4.2×10⁹ or more Treg, approximately 4.3×10⁹ or more Treg, approximately 4.4×10⁹ or more Treg, approximately 4.5×10⁹ or more Treg, approximately 4.6×10⁹ or more Treg, approximately 4.7×10⁹ or more Treg, approximately 4.8×10⁹ or more Treg, approximately 4.9×10⁹ or more Treg, or approximately 5.0×10⁹ or more Treg. In some embodiments, the Treg are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim}FOXP3⁺, CD3+FOXP3⁺, CD3⁺CD4+FOXP3⁺, CD3⁺CD4⁺CD25+FOXP3+, CD3⁺CD25+FOXP3⁺, CD3⁺CD25⁺CD127^{dim}, CD4⁺CD25⁺, CD4⁺CD25⁺CD127^{dim}FOXP3+, FOXP3+, CD4+FOXP3⁺, CD4⁺CD25+FOXP3⁺, CD25+FOXP3+, or CD25⁺CD127^{dim}.

A population of cells enriched for Treg, or comprising at least one dose of Treg, can have a defined level of purity for Treg cells. For example, a population of cells enriched for Treg of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Treg cells as a percentage of total cells (e.g., where Treg are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells of the present disclosure enriched for Treg, or comprising at least one dose of Treg, can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95% Treg as a percentage of total cells, nucleated cells, or CD45⁺ cells (e.g., where the Treg are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells of the present disclosure enriched for Treg, or comprising at least one dose of Treg, can have a defined level of contaminating non-Treg cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Treg cells per kg of the human subject's actual body weight or (adjusted) ideal body weight are present in a population of cells enriched for Treg of the disclosure, where non-Treg cells are FOXP3− or CD127+/bright.

In some embodiments, a population of cells of the present disclosure enriched for Treg, or comprising at least one does of Treg, comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Treg cells, where non-Treg cells are FOXP3− or CD127+/bright.

In various embodiments, the population of cells enriched for Treg, or comprising at least one dose of Treg, comprises less than about 5 EU of endotoxins per ml of the solution, less than about 1 EU of endotoxins per ml of the solution, and/or less than about 0.5 EU of endotoxins per ml of the solution.

In some embodiments, at least one of the cell populations comprises less than about 5 EU of endotoxins/ml of respective suspension liquid.

A population of cells enriched for Treg, or comprising at least one dose of Treg, can comprise 0.5 EU/ml endotoxins to 10 EU/ml endotoxins. A population of cells enriched for Treg, or comprising at least one dose of Treg, can comprise at least 0.5 EU/ml endotoxins. A population of cells enriched for Treg, or comprising at least one dose of Treg, can comprise at most 10 EU/ml endotoxins. A population of cells enriched for Treg, or comprising at least one dose of Treg, can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A population of cells enriched for Treg, or comprising at least one dose of Treg, can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Treg can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Treg can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A population of cells enriched for Treg, or comprising at least one dose of Treg, can comprise 0.5% w/w to 10% w/w unbound reagents. These unbound reagents may include any affinity reagents used for the sorting of Treg, for instance, antibodies, or purification particles or magnetic particles. A population of cells enriched for Treg, or comprising at least one dose of Treg, can comprise at least 0.5% w/w unbound reagents. A population of cells enriched for Treg can comprise at most 10% w/w unbound reagents. A population of cells enriched for Treg, or comprising at least one dose of Treg, can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A population of cells enriched for Treg, or comprising at least one dose of Treg, can comprise 10% w/w, 8% w/w, 6% w/w, 5% w/w, 4% w/w, 2% w/w, 1% w/w, or 0.5% w/w unbound reagents. A population of cells enriched for Treg, or comprising at least one dose of Treg, can comprise at least 8% w/w, 6% w/w, 5% w/w, 4% w/w, 2% w/w, 1% w/w, or 0.5% w/w unbound reagents. A population of cells enriched for Treg, or comprising at least one dose of Treg, can comprise at most 10% w/w, 8% w/w, 6% w/w, 5% w/w, 4% w/w, 2% w/w or 1% w/w unbound reagents.

A population of cells enriched for Treg, or comprising at least one dose of Treg, can comprise 1×10³ to 50×10³ microbeads per cell. These microbeads may comprise microbeads used to purify the Treg population, for instance, an anti-CD25 antibody comprising microbead used to sort the Treg population, or an anti-CD4 antibody comprising microbead used to sort the Treg population, and/or an anti-CD127 antibody comprising microbead used to sort the Treg population. A population of cells enriched for Treg, or comprising at least one dose of Treg, can comprise at least 1×10³ microbeads per cell. A population of cells enriched for Treg, or comprising at least one dose of Treg, can comprise at most 50×10³ microbeads per cell. A population of cells enriched for Treg, or comprising at least one dose of Treg, can comprise 50×10³ to 40×10³, 50×10³ to 30×10³, 50×10³ to 20×10³, 50×10³ to 10×10³, 50×10³ to 5×10³, 50×10³ to 4×10³, 50×10³ to 2×10³, 50×10³ to 1×10³, 40×10³ to 30×10³, 40×10³ to 20×10³, 40×10³ to 10×10³, 40×10³ to 5×10³, 40×10³ to 4×10³, 40×10³ to 2×10³, 40×10³ to 1×10³, 30×10³ to 20×10³, 30×10³ to 10×10³, 30×10³ to 5×10³, 30×10³ to 4×10³, 30×10³ to 2×10³, 30×10³ to 1×10³, 20×10³ to 10×10³, 20×10³ to 5×10³, 20×10³ to 4×10³, 20×10³ to 2×10³, 20×10³ to 1×10³, 10×10³ to 5×10³, 10×10³ to 4×10³, 10×10³ to 2×10³, 10×10³ to 1×10³, 5×10³ to 4×10³, 5×10³ to 2×10³, 5×10³ to 1×10³, 4×10³ to 2×10³, 4×10³ to 1×10³, or 2×10³ to 1×10³ microbeads per cell. A population of cells enriched for Treg, or comprising at least one dose of Treg, can comprise 50×10³, 40×10³, 30×10³, 20×10³, 10×10³, 5×10³, 4×10³, 2×10³, or 1×10³ microbeads per cell.

In some embodiments, a population of cells of the present disclosure enriched for Treg, or comprising at least one dose of Treg, can comprise a population of naïve Treg cells and/or a population of memory Treg cells.

As used herein, the term “naïve Treg” refers to one or more non-antigen experienced regulatory T cells that express the phenotypic markers CD4, CD25, and CD45RA, and do not express or have low expression of CD45RO and CD127. Without wishing to be bound by theory, it is believed that naïve Treg may be advantageous because the cells have higher plasticity for responding to antigens than antigen experienced Treg. In addition, naïve Treg have increased longevity compared to antigen experienced Treg.

In some embodiments, the population of cells of the present disclosure enriched for Treg, or comprising at least one dose of Treg, can comprise a population of naïve Treg, or at least one dose of naïve Treg, comprising from approximately 2.0×10⁵ to approximately 5.0×10⁸ naïve Treg cells per kilogram of actual body weight or ideal body weight of a human subject receiving a treatment or cellular therapy product of the present disclosure. In some embodiments, the population of cells of the present disclosure enriched for Treg, or comprising at least one dose of Treg, can comprise a population of naïve Treg, or at least one dose of naïve Treg, comprising a total dose of approximately 8.0×10⁵ to approximately 1.0×10¹¹ naïve Treg cells.

A population of cells enriched for Treg, or comprising at least one dose of Treg, can comprise a population of naïve Treg, or at least one dose of naïve Treg, comprising at least about 2×10⁵, at least about 2.5×10⁵, at least about 3×10⁵, at least about 3.5×10⁵, at least about 4×10⁵, at least about 4.5×10⁵, at least about 5×10⁵, at least about 5.5×10⁵, at least about 6×10⁵, at least about 6.5×10⁵, at least about 7×10⁵, at least about 7.5×10⁵, at least about 8×10⁵, at least about 8.5×10⁵, at least about 9×10⁵, at least about 9.5×10⁵, at least about 1×10⁶, at least about 1.5×10⁶, at least about 2×10⁶, at least about 2.5×10⁶, at least about 3×10⁶, at least about 3.5×10⁶, at least about 4×10⁶, at least about 4.5×10⁶, at least about 5×10⁶, at least about 5.5×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, or more naïve Treg cells per kg of the human subject's actual body weight or (adjusted) ideal body weight. In some embodiments, the naïve Treg are CD4⁺CD25⁺CD127^{dim}FoxP3⁺CD45RA⁺CD45RO⁻.

In some embodiments, the population of cells enriched for Treg, or comprising at least one dose of Treg, can comprise a population of naïve Treg, or at least one dose of naïve Treg, comprising approximately 8.0×10⁵ or more naïve Treg, approximately 8.5×10⁵ or more naïve Treg, approximately 9.0×10⁵ or more naïve Treg, approximately 9.5×10⁵ or more naïve Treg, approximately 1.0×10⁶ or more naïve Treg, approximately 1.5×10⁶ or more naïve Treg, approximately 2.0×10⁶ or more naïve Treg, approximately 2.5×10⁶ or more naïve Treg, approximately 3.0×10⁶ or more naïve Treg, approximately 3.5×10⁶ or more naïve Treg, approximately 4.0×10⁶ or more naïve Treg, approximately 4.5×10⁶ or more naïve Treg, approximately 5.0×10⁶ or more naïve Treg, approximately 5.5×10⁶ or more naïve Treg, approximately 6.0×10⁶ or more naïve Treg, approximately 6.5×10⁶ or more naïve Treg, approximately 7.0×10⁶ or more naïve Treg, approximately 7.5×10⁶ or more naïve Treg, approximately 8.0×10⁶ or more naïve Treg, approximately 8.5×10⁶ or more naïve Treg, approximately 9.0×10⁶ or more naïve Treg, approximately 9.5×10⁶ or more naïve Treg, approximately 1.0×10⁷ or more naïve Treg, approximately 1.5×10⁷ or more naïve Treg, approximately 2.0×10⁷ or more naïve Treg, approximately 2.5×10⁷ or more naïve Treg, approximately 3.0×10⁷ or more naïve Treg, approximately 3.5×10⁷ or more naïve Treg, approximately 4.0×10⁷ or more naïve Treg, approximately 4.5×10⁷ or more naïve Treg, approximately 5.0×10⁷ or more naïve Treg, approximately 5.5×10⁷ or more naïve Treg, approximately 6.0×10⁷ or more naïve Treg, approximately 6.5×10⁷ or more naïve Treg, approximately 7.0×10⁷ or more naïve Treg, approximately 7.5×10⁷ or more naïve Treg, approximately 8.0×10⁷ or more naïve Treg, approximately 8.5×10⁷ or more naïve Treg, approximately 9.0×10⁷ or more naïve Treg, approximately 9.5×10⁷ or more naïve Treg, approximately 1.0×10⁸ or more naïve Treg, approximately 1.5×10⁸ or more naïve Treg, approximately 2.0×10⁸ or more naïve Treg, approximately 2.5×10⁸ or more naïve Treg, approximately 3.0×10⁸ or more naïve Treg, approximately 3.5×10⁸ or more naïve Treg, approximately 4.0×10⁸ or more naïve Treg, approximately 4.5×10⁸ or more naïve Treg, approximately 5.0×10⁸ or more naïve Treg, approximately 5.5×10⁸ or more naïve Treg, approximately 6.0×10⁸ or more naïve Treg, approximately 6.5×10⁸ or more naïve Treg, approximately 7.0×10⁸ or more naïve Treg, approximately 7.5×10⁸ or more naïve Treg, approximately 8.0×10⁸ or more naïve Treg, approximately 8.5×10⁸ or more naïve Treg, approximately 9.0×10⁸ or more naïve Treg, approximately 9.5×10⁸ or more naïve Treg, approximately 1.0×10⁹ or more naïve Treg, approximately 1.5×10⁹ or more naïve Treg, approximately 2.0×10⁹ or more naïve Treg, approximately 2.5×10⁹ or more naïve Treg, approximately 3.0×10⁹ or more naïve Treg, approximately 3.5×10⁹ or more naïve Treg, approximately 4.0×10⁹ or more naïve Treg, approximately 4.5×10⁹ or more naïve Treg, approximately 5.0×10⁹ or more naïve Treg, approximately 5.5×10⁹ or more naïve Treg, approximately 6.0×10⁹ or more naïve Treg, approximately 6.5×10⁹ or more naïve Treg, approximately 7.0×10⁹ or more naïve Treg, approximately 7.5×10⁹ or more naïve Treg, approximately 8.0×10⁹ or more naïve Treg, approximately 8.5×10⁹ or more naïve Treg, approximately 9.0×10⁹ or more naïve Treg, approximately 9.5×10⁹ or more naïve Treg, approximately 1.0×10¹⁰ or more naïve Treg, approximately 1.5×10¹⁰ or more naïve Treg, approximately 2.0×10¹⁰ or more naïve Treg, approximately 2.5×10¹⁰ or more naïve Treg, approximately 3.0×10¹⁰ or more naïve Treg, approximately 3.5×10¹⁰ or more naïve Treg, approximately 4.0×10¹⁰ or more naïve Treg, approximately 4.5×10¹⁰ or more naïve Treg, approximately 5.0×10¹⁰ or more naïve Treg, approximately 5.5×10¹⁰ or more naïve Treg, approximately 6.0×10¹⁰ or more naïve Treg, approximately 6.5×10¹⁰ or more naïve Treg, approximately 7.0×10¹⁰ or more naïve Treg, approximately 7.5×10¹⁰ or more naïve Treg, approximately 8.0×10¹⁰ or more naïve Treg, approximately 8.5×10¹⁰ or more naïve Treg, approximately 9.0×10¹⁰ or more naïve Treg, approximately 9.5×10¹⁰ or more naïve Treg, or approximately 1.0×10¹¹ or more naïve Treg. In some embodiments, the naïve Treg are CD4⁺CD25⁺CD127^{dim}FoxP3⁺CD45RA⁺CD45RO⁻.

As used herein, the term “memory Treg” refers to one or more antigen experienced regulatory T cells that are capable of providing suppressive effects on autoimmunity and express the phenotypic markers CD4, CD25, and CD45RO and do not express or have low expression of CD127 and CD45RA.

In some embodiments, the population of cells of the present disclosure enriched for Treg, or comprising at least one dose of Treg, can comprise a population of memory Treg, or at least one dose of memory Treg, comprising from approximately 5.0×10⁴ to approximately 5.0×10⁸ memory Treg cells per kilogram of actual body weight or ideal body weight of a human subject receiving a treatment or cellular therapy product of the present disclosure. In some embodiments, the population of cells of the present disclosure enriched for Treg, or comprising at least one dose of Treg, can comprise a population of memory Treg, or at least one dose of memory Treg, comprising a total dose of approximately 2.0×10⁵ to approximately 1.0×10¹¹ memory Treg cells.

A population of cells enriched for Treg, or comprising at least one dose of Treg, can comprise a population of memory Treg, or at least one dose of memory Treg, comprising at least about 5×10⁴, at least about 5.5×10⁴, at least about 6×10⁴, at least about 6.5×10⁴, at least about 7×10⁴, at least about 7.5×10⁴, at least about 8×10⁴, at least about 8.5×10⁴, at least about 9×10⁴, at least about 9.5×10⁴, at least about 1×10⁵, at least about 1.5×10⁵, at least about 2×10⁵, at least about 2.5×10⁵, at least about 3×10⁵, at least about 3.5×10⁵, at least about 4×10⁵, at least about 4.5×10⁵, at least about 5×10⁵, at least about 5.5×10⁵, at least about 6×10⁵, at least about 6.5×10⁵, at least about 7×10⁵, at least about 7.5×10⁵, at least about 8×10⁵, at least about 8.5×10⁵, at least about 9×10⁵, at least about 9.5×10⁵, at least about 1×10⁶, at least about 1.5×10⁶, at least about 2×10⁶, at least about 2.5×10⁶, at least about 3×10⁶, at least about 3.5×10⁶, at least about 4×10⁶, at least about 4.5×10⁶, at least about 5×10⁶, at least about 5.5×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, or more memory Treg cells per kg of the human subject's actual body weight or (adjusted) ideal body weight. In some embodiments, the memory Treg are CD4⁺CD25⁺CD127^{dim}FoxP3TCD45RA⁻CD45RO⁺.

In some embodiments, the population of cells enriched for Treg, or comprising at least one dose of Treg, can comprise a population of memory Treg, or at least one dose of memory Treg, comprising approximately 2.0×10⁵ or more memory Treg, approximately 2.5×10⁵ or more memory Treg, approximately 3.0×10⁵ or more memory Treg, approximately 3.5×10⁵ or more memory Treg, approximately 4.0×10⁵ or more memory Treg, approximately 4.5×10⁵ or more memory Treg, approximately 5.0×10⁵ or more memory Treg, approximately 5.5×10⁵ or more memory Treg, approximately 6.0×10⁵ or more memory Treg, approximately 6.5×10⁵ or more memory Treg, approximately 7.0×10⁵ or more memory Treg, approximately 7.5×10⁵ or more memory Treg, approximately 8.0×10⁵ or more memory Treg, approximately 8.5×10⁵ or more memory Treg, approximately 9.0×10⁵ or more memory Treg, approximately 9.5×10⁵ or more memory Treg, approximately 1.0×10⁶ or more memory Treg, approximately 1.5×10⁶ or more memory Treg, approximately 2.0×10⁶ or more memory Treg, approximately 2.5×10⁶ or more memory Treg, approximately 3.0×10⁶ or more memory Treg, approximately 3.5×10⁶ or more memory Treg, approximately 4.0×10⁶ or more memory Treg, approximately 4.5×10⁶ or more memory Treg, approximately 5.0×10⁶ or more memory Treg, approximately 5.5×10⁶ or more memory Treg, approximately 6.0×10⁶ or more memory Treg, approximately 6.5×10⁶ or more memory Treg, approximately 7.0×10⁶ or more memory Treg, approximately 7.5×10⁶ or more memory Treg, approximately 8.0×10⁶ or more memory Treg, approximately 8.5×10⁶ or more memory Treg, approximately 9.0×10⁶ or more memory Treg, approximately 9.5×10⁶ or more memory Treg, approximately 1.0×10⁷ or more memory Treg, approximately 1.5×10⁷ or more memory Treg, approximately 2.0×10⁷ or more memory Treg, approximately 2.5×10⁷ or more memory Treg, approximately 3.0×10⁷ or more memory Treg, approximately 3.5×10⁷ or more memory Treg, approximately 4.0×10⁷ or more memory Treg, approximately 4.5×10⁷ or more memory Treg, approximately 5.0×10⁷ or more memory Treg, approximately 5.5×10⁷ or more memory Treg, approximately 6.0×10⁷ or more memory Treg, approximately 6.5×10⁷ or more memory Treg, approximately 7.0×10⁷ or more memory Treg, approximately 7.5×10⁷ or more memory Treg, approximately 8.0×10⁷ or more memory Treg, approximately 8.5×10⁷ or more memory Treg, approximately 9.0×10⁷ or more memory Treg, approximately 9.5×10⁷ or more memory Treg, approximately 1.0×10⁸ or more memory Treg, approximately 1.5×10⁸ or more memory Treg, approximately 2.0×10⁸ or more memory Treg, approximately 2.5×10⁸ or more memory Treg, approximately 3.0×10⁸ or more memory Treg, approximately 3.5×10⁸ or more memory Treg, approximately 4.0×10⁸ or more memory Treg, approximately 4.5×10⁸ or more memory Treg, approximately 5.0×10⁸ or more memory Treg, approximately 5.5×10⁸ or more memory Treg, approximately 6.0×10⁸ or more memory Treg, approximately 6.5×10⁸ or more memory Treg, approximately 7.0×10⁸ or more memory Treg, approximately 7.5×10⁸ or more memory Treg, approximately 8.0×10⁸ or more memory Treg, approximately 8.5×10⁸ or more memory Treg, approximately 9.0×10⁸ or more memory Treg, approximately 9.5×10⁸ or more memory Treg, approximately 1.0×10⁹ or more memory Treg, approximately 1.5×10⁹ or more memory Treg, approximately 2.0×10⁹ or more memory Treg, approximately 2.5×10⁹ or more memory Treg, approximately 3.0×10⁹ or more memory Treg, approximately 3.5×10⁹ or more memory Treg, approximately 4.0×10⁹ or more memory Treg, approximately 4.5×10⁹ or more memory Treg, approximately 5.0×10⁹ or more memory Treg, approximately 5.5×10⁹ or more memory Treg, approximately 6.0×10⁹ or more memory Treg, approximately 6.5×10⁹ or more memory Treg, approximately 7.0×10⁹ or more memory Treg, approximately 7.5×10⁹ or more memory Treg, approximately 8.0×10⁹ or more memory Treg, approximately 8.5×10⁹ or more memory Treg, approximately 9.0×10⁹ or more memory Treg, approximately 9.5×10⁹ or more memory Treg, approximately 1.0×10¹⁰ or more memory Treg, approximately 1.5×10¹⁰ or more memory Treg, approximately 2.0×10¹⁰ or more memory Treg, approximately 2.5×10¹⁰ or more memory Treg, approximately 3.0×10¹⁰ or more memory Treg, approximately 3.5×10¹⁰ or more memory Treg, approximately 4.0×10¹⁰ or more memory Treg, approximately 4.5×10¹⁰ or more memory Treg, approximately 5.0×10¹⁰ or more memory Treg, approximately 5.5×10¹⁰ or more memory Treg, approximately 6.0×10¹⁰ or more memory Treg, approximately 6.5×10¹⁰ or more memory Treg, approximately 7.0×10¹⁰ or more memory Treg, approximately 7.5×10¹⁰ or more memory Treg, approximately 8.0×10¹⁰ or more memory Treg, approximately 8.5×10¹⁰ or more memory Treg, approximately 9.0×10¹⁰ or more memory Treg, approximately 9.5×10¹⁰ or more memory Treg, or approximately 1.0×10¹¹ or more memory Treg. In some embodiments, the memory Treg are CD4⁺CD25⁺CD127^{dim}FoxP3⁺CD45RA-CD45RO⁺. iNKT

In some embodiments, a population of cells of the present disclosure comprising iNKT, e.g., at least one dose of iNKT or an enriched population of iNKT, can comprise from about 1×10² to about 1×10⁹ iNKT per kilogram of actual body weight or ideal body weight of a human subject receiving a treatment or cellular therapy product of the present disclosure. In some embodiments, the population of cells comprising iNKT, e.g., at least one dose of iNKT or an enriched population of iNKT, can comprise from about 1×10² to about 1×10⁹ iNKT per kilogram of IBW of the human subject.

A population of cells comprising iNKT, at least one dose of iNKT, or an enriched population of iNKT, can comprise at least about 1×10², at least about 2×10², at least about 3×10², at least about 4×10², at least about 5×10², at least about 6×10², at least about 7×10², at least about 8×10², at least about 9×10², at least about 1×10², at least about 2×10², at least about 3×10², at least about 4×10², at least about 5×10², at least about 6×10², at least about 7×10², at least about 8×10³, at least about 9×10³, at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more iNKT per kg of the human subject's actual body weight or (adjusted) ideal body weight (e.g., where iNKT are CD3+Vα24Jα18+).

A population of cells comprising iNKT, at least one dose of iNKT, or an enriched population of iNKT, can comprise at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, at most about 1×10⁹, or more iNKT per kg of the human subject's actual body weight or (adjusted) ideal body weight (e.g., where iNKT are CD3+Vα24Jα18+).

For example, a population of cells comprising iNKT, at least one dose of iNKT, or an enriched population of iNKT, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ iNKT per kg of the human subject's actual body weight or (adjusted) ideal body weight (e.g., where iNKT are CD3+Vα24Jα18+).

A population of cells comprising iNKT, at least one dose of iNKT, or an enriched population of iNKT, can comprise approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT (e.g., where iNKT are CD3+Vα24Jα18+).

A population of cells of the present disclosure comprising iNKT, at least one dose of iNKT, or an enriched population of iNKT, can have a defined level of purity for iNKT cells. For example, a population of iNKT of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more iNKT cells as a percentage of total cells. (e.g., where iNKT are CD3+Vα24Jα18+).

A population of cells of the present disclosure comprising iNKT, at least one dose of iNKT, or an enriched population of iNKT, can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, iNKT as a percentage of total cells. (e.g., where iNKT are CD3+Vα24Jα18+).

A population of cells of the present disclosure comprising iNKT, at least one dose of iNKT, or an enriched population of iNKT, can have a defined level of contaminating non-iNKT cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-iNKT cells per kg of recipient subject's actual body weight or ideal body weight are present in a population of iNKT of the disclosure, (e.g., where non-iNKT cells are Vα24Jα18−).

In some embodiments, a population of cells of the present disclosure comprising iNKT, at least one dose of iNKT, or an enriched population of iNKT, comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-iNKT cells, (e.g., where non-iNKT cells are Vα24Jα18−).

### Tmem

In some embodiments, a population of cells of the present disclosure comprising Tmem, or comprising at least one dose of Tmem, comprises from about 1×10⁴ to about 1×10⁹ Tmem per kilogram of actual body weight or ideal body weight of a human subject receiving a treatment or cellular therapy product of the present disclosure. In some embodiments, the population of cells comprising Tmem, or comprising at least one dose of Tmem, comprises from about 1×10⁴ to about 1×10⁹ Tmem per kilogram of IBW of the human subject (e.g., where Tmem are CD3+CD45RA− CD45RO+).

A population of cells of the present disclosure comprising Tmem, or comprising at least one dose of Tmem, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tmem per kilogram (kg) of the human subject's actual body weight or (adjusted) ideal body weight (e.g., where Tmem are CD3⁺CD45RA-CD45RO+).

A population of cells of the present disclosure comprising Tmem, or comprising at least one dose of Tmem, can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tmem per kg of the human subject's actual body weight or (adjusted) ideal body weight (e.g., where Tmem are CD3+CD45RA−CD45RO+).

For example, a population of cells of the present disclosure comprising Tmem, or comprising at least one dose of Tmem, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁶, 5×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tmem per kg of the human subject's actual body weight or (adjusted) ideal body weight (e.g., where Tmem are CD3⁺CD45RA−CD45RO⁺).

In some embodiments, a population of cells of the present disclosure comprising Tmem, or comprising at least one dose of Tmem, can comprise from approximately 1.0×10⁵ to approximately 5.0×10¹⁰, from approximately 5.0×10⁵ to approximately 1.5×10¹⁰, from approximately 5.0×10⁵ to approximately 5.0×10⁸, or from approximately 1.5×10⁷ to approximately 1.5×10¹⁰ Tmem. In some embodiments, a population of Tmem, or a dose of Tmem, can comprise approximately 1.0×10⁵ or more Tmem, approximately 2.0×10⁵ or more Tmem, approximately 3.0×10⁵ or more Tmem, approximately 4.0×10⁵ or more Tmem, approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.0×10⁶ or more Tmem, approximately 1.1×10⁶ or more Tmem, approximately 1.2×10⁶ or more Tmem, approximately 1.3×10⁶ or more Tmem, approximately 1.4×10⁶ or more Tmem, approximately 1.5×10⁶ or more Tmem, approximately 1.6×10⁶ or more Tmem, approximately 1.7×10⁶ or more Tmem, approximately 1.8×10⁶ or more Tmem, approximately 1.9×10⁶ or more Tmem, approximately 2.0×10⁶ or more Tmem, approximately 2.1×10⁶ or more Tmem, approximately 2.2×10⁶ or more Tmem, approximately 2.3×10⁶ or more Tmem, approximately 2.4×10⁶ or more Tmem, approximately 2.5×10⁶ or more Tmem, approximately 2.6×10⁶ or more Tmem, approximately 2.7×10⁶ or more Tmem, approximately 2.8×10⁶ or more Tmem, approximately 2.9×10⁶ or more Tmem, approximately 3.0×10⁶ or more Tmem, approximately 3.1×10⁶ or more Tmem, approximately 3.2×10⁶ or more Tmem, approximately 3.3×10⁶ or more Tmem, approximately 3.4×10⁶ or more Tmem, approximately 3.5×10⁶ or more Tmem, approximately 3.6×10⁶ or more Tmem, approximately 3.7×10⁶ or more Tmem, approximately 3.8×10⁶ or more Tmem, approximately 3.9×10⁶ or more Tmem, approximately 4.0×10⁶ or more Tmem, approximately 4.1×10⁶ or more Tmem, approximately 4.2×10⁶ or more Tmem, approximately 4.3×10⁶ or more Tmem, approximately 4.4×10⁶ or more Tmem, approximately 4.5×10⁶ or more Tmem, approximately 4.6×10⁶ or more Tmem, approximately 4.7×10⁶ or more Tmem, approximately 4.8×10⁶ or more Tmem, approximately 4.9×10⁶ or more Tmem, approximately 5.0×10⁶ or more Tmem, approximately 5.1×10⁶ or more Tmem, approximately 5.2×10⁶ or more Tmem, approximately 5.3×10⁶ or more Tmem, approximately 5.4×10⁶ or more Tmem, approximately 5.5×10⁶ or more Tmem, approximately 5.6×10⁶ or more Tmem, approximately 5.7×10⁶ or more Tmem, approximately 5.8×10⁶ or more Tmem, approximately 5.9×10⁶ or more Tmem, approximately 6.0×10⁶ or more Tmem, approximately 6.1×10⁶ or more Tmem, approximately 6.2×10⁶ or more Tmem, approximately 6.3×10⁶ or more Tmem, approximately 6.4×10⁶ or more Tmem, approximately 6.5×10⁶ or more Tmem, approximately 6.6×10⁶ or more Tmem, approximately 6.7×10⁶ or more Tmem, approximately 6.8×10⁶ or more Tmem, approximately 6.9×10⁶ or more Tmem, approximately 7.0×10⁶ or more Tmem, approximately 7.1×10⁶ or more Tmem, approximately 7.2×10⁶ or more Tmem, approximately 7.3×10⁶ or more Tmem, approximately 7.4×10⁶ or more Tmem, approximately 7.5×10⁶ or more Tmem, approximately 7.6×10⁶ or more Tmem, approximately 7.7×10⁶ or more Tmem, approximately 7.8×10⁶ or more Tmem, approximately 7.9×10⁶ or more Tmem, approximately 8.0×10⁶ or more Tmem, approximately 8.1×10⁶ or more Tmem, approximately 8.2×10⁶ or more Tmem, approximately 8.3×10⁶ or more Tmem, approximately 8.4×10⁶ or more Tmem, approximately 8.5×10⁶ or more Tmem, approximately 8.6×10⁶ or more Tmem, approximately 8.7×10⁶ or more Tmem, approximately 8.8×10⁶ or more Tmem, approximately 8.9×10⁶ or more Tmem, approximately 9.0×10⁶ or more Tmem, approximately 9.1×10⁶ or more Tmem, approximately 9.2×10⁶ or more Tmem, approximately 9.3×10⁶ or more Tmem, approximately 9.4×10⁶ or more Tmem, approximately 9.5×10⁶ or more Tmem, approximately 9.6×10⁶ or more Tmem, approximately 9.7×10⁶ or more Tmem, approximately 9.8×10⁶ or more Tmem, approximately 9.9×10⁶ or more Tmem, approximately 1.0×10⁷ or more Tmem, approximately 1.1×10⁷ or more Tmem, approximately 1.2×10⁷ or more Tmem, approximately 1.3×10⁷ or more Tmem, approximately 1.4×10⁷ or more Tmem, approximately 1.5×10⁷ or more Tmem, approximately 1.6×10⁷ or more Tmem, approximately 1.7×10⁷ or more Tmem, approximately 1.8×10⁷ or more Tmem, approximately 1.9×10⁷ or more Tmem, approximately 2.0×10⁷ or more Tmem, approximately 2.1×10⁷ or more Tmem, approximately 2.2×10⁷ or more Tmem, approximately 2.3×10⁷ or more Tmem, approximately 2.4×10⁷ or more Tmem, approximately 2.5×10⁷ or more Tmem, approximately 2.6×10⁷ or more Tmem, approximately 2.7×10⁷ or more Tmem, approximately 2.8×10⁷ or more Tmem, approximately 2.9×10⁷ or more Tmem, approximately 3.0×10⁷ or more Tmem, approximately 3.1×10⁷ or more Tmem, approximately 3.2×10⁷ or more Tmem, approximately 3.3×10⁷ or more Tmem, approximately 3.4×10⁷ or more Tmem, approximately 3.5×10⁷ or more Tmem, approximately 3.6×10⁷ or more Tmem, approximately 3.7×10⁷ or more Tmem, approximately 3.8×10⁷ or more Tmem, approximately 3.9×10⁷ or more Tmem, approximately 4.0×10⁷ or more Tmem, approximately 4.1×10⁷ or more Tmem, approximately 4.2×10⁷ or more Tmem, approximately 4.3×10⁷ or more Tmem, approximately 4.4×10⁷ or more Tmem, approximately 4.5×10⁷ or more Tmem, approximately 4.6×10⁷ or more Tmem, approximately 4.7×10⁷ or more Tmem, approximately 4.8×10⁷ or more Tmem, approximately 4.9×10⁷ or more Tmem, approximately 5.0×10⁷ or more Tmem, approximately 5.1×10⁷ or more Tmem, approximately 5.2×10⁷ or more Tmem, approximately 5.3×10⁷ or more Tmem, approximately 5.4×10⁷ or more Tmem, approximately 5.5×10⁷ or more Tmem, approximately 5.6×10⁷ or more Tmem, approximately 5.7×10⁷ or more Tmem, approximately 5.8×10⁷ or more Tmem, approximately 5.9×10⁷ or more Tmem, approximately 6.0×10⁷ or more Tmem, approximately 6.1×10⁷ or more Tmem, approximately 6.2×10⁷ or more Tmem, approximately 6.3×10⁷ or more Tmem, approximately 6.4×10⁷ or more Tmem, approximately 6.5×10⁷ or more Tmem, approximately 6.6×10⁷ or more Tmem, approximately 6.7×10⁷ or more Tmem, approximately 6.8×10⁷ or more Tmem, approximately 6.9×10⁷ or more Tmem, approximately 7.0×10⁷ or more Tmem, approximately 7.1×10⁷ or more Tmem, approximately 7.2×10⁷ or more Tmem, approximately 7.3×10⁷ or more Tmem, approximately 7.4×10⁷ or more Tmem, approximately 7.5×10⁷ or more Tmem, approximately 7.6×10⁷ or more Tmem, approximately 7.7×10⁷ or more Tmem, approximately 7.8×10⁷ or more Tmem, approximately 7.9×10⁷ or more Tmem, approximately 8.0×10⁷ or more Tmem, approximately 8.1×10⁷ or more Tmem, approximately 8.2×10⁷ or more Tmem, approximately 8.3×10⁷ or more Tmem, approximately 8.4×10⁷ or more Tmem, approximately 8.5×10⁷ or more Tmem, approximately 8.6×10⁷ or more Tmem, approximately 8.7×10⁷ or more Tmem, approximately 8.8×10⁷ or more Tmem, approximately 8.9×10⁷ or more Tmem, approximately 9.0×10⁷ or more Tmem, approximately 9.1×10⁷ or more Tmem, approximately 9.2×10⁷ or more Tmem, approximately 9.3×10⁷ or more Tmem, approximately 9.4×10⁷ or more Tmem, approximately 9.5×10⁷ or more Tmem, approximately 9.6×10⁷ or more Tmem, approximately 9.7×10⁷ or more Tmem, approximately 9.8×10⁷ or more Tmem, approximately 9.9×10⁷ or more Tmem, approximately 1.0×10⁸ or more Tmem, approximately 1.1×10⁸ or more Tmem, approximately 1.2×10⁸ or more Tmem, approximately 1.3×10⁸ or more Tmem, approximately 1.4×10⁸ or more Tmem, approximately 1.5×10⁸ or more Tmem, approximately 1.6×10⁸ or more Tmem, approximately 1.7×10⁸ or more Tmem, approximately 1.8×10⁸ or more Tmem, approximately 1.9×10⁸ or more Tmem, approximately 2.0×10⁸ or more Tmem, approximately 2.1×10⁸ or more Tmem, approximately 2.2×10⁸ or more Tmem, approximately 2.3×10⁸ or more Tmem, approximately 2.4×10⁸ or more Tmem, approximately 2.5×10⁸ or more Tmem, approximately 2.6×10⁸ or more Tmem, approximately 2.7×10⁸ or more Tmem, approximately 2.8×10⁸ or more Tmem, approximately 2.9×10⁸ or more Tmem, approximately 3.0×10⁸ or more Tmem, approximately 3.1×10⁸ or more Tmem, approximately 3.2×10⁸ or more Tmem, approximately 3.3×10⁸ or more Tmem, approximately 3.4×10⁸ or more Tmem, approximately 3.5×10⁸ or more Tmem, approximately 3.6×10⁸ or more Tmem, approximately 3.7×10⁸ or more Tmem, approximately 3.8×10⁸ or more Tmem, approximately 3.9×10⁸ or more Tmem, approximately 4.0×10⁸ or more Tmem, approximately 4.1×10⁸ or more Tmem, approximately 4.2×10⁸ or more Tmem, approximately 4.3×10⁸ or more Tmem, approximately 4.4×10⁸ or more Tmem, approximately 4.5×10⁸ or more Tmem, approximately 4.6×10⁸ or more Tmem, approximately 4.7×10⁸ or more Tmem, approximately 4.8×10⁸ or more Tmem, approximately 4.9×10⁸ or more Tmem, approximately 5.0×10⁸ or more Tmem, approximately 5.1×10⁸ or more Tmem, approximately 5.2×10⁸ or more Tmem, approximately 5.3×10⁸ or more Tmem, approximately 5.4×10⁸ or more Tmem, approximately 5.5×10⁸ or more Tmem, approximately 5.6×10⁸ or more Tmem, approximately 5.7×10⁸ or more Tmem, approximately 5.8×10⁸ or more Tmem, approximately 5.9×10⁸ or more Tmem, approximately 6.0×10⁸ or more Tmem, approximately 6.1×10⁸ or more Tmem, approximately 6.2×10⁸ or more Tmem, approximately 6.3×10⁸ or more Tmem, approximately 6.4×10⁸ or more Tmem, approximately 6.5×10⁸ or more Tmem, approximately 6.6×10⁸ or more Tmem, approximately 6.7×10⁸ or more Tmem, approximately 6.8×10⁸ or more Tmem, approximately 6.9×10⁸ or more Tmem, approximately 7.0×10⁸ or more Tmem, approximately 7.1×10⁸ or more Tmem, approximately 7.2×10⁸ or more Tmem, approximately 7.3×10⁸ or more Tmem, approximately 7.4×10⁸ or more Tmem, approximately 7.5×10⁸ or more Tmem, approximately 7.6×10⁸ or more Tmem, approximately 7.7×10⁸ or more Tmem, approximately 7.8×10⁸ or more Tmem, approximately 7.9×10⁸ or more Tmem, approximately 8.0×10⁸ or more Tmem, approximately 8.1×10⁸ or more Tmem, approximately 8.2×10⁸ or more Tmem, approximately 8.3×10⁸ or more Tmem, approximately 8.4×10⁸ or more Tmem, approximately 8.5×10⁸ or more Tmem, approximately 8.6×10⁸ or more Tmem, approximately 8.7×10⁸ or more Tmem, approximately 8.8×10⁸ or more Tmem, approximately 8.9×10⁸ or more Tmem, approximately 9.0×10⁸ or more Tmem, approximately 9.1×10⁸ or more Tmem, approximately 9.2×10⁸ or more Tmem, approximately 9.3×10⁸ or more Tmem, approximately 9.4×10⁸ or more Tmem, approximately 9.5×10⁸ or more Tmem, approximately 9.6×10⁸ or more Tmem, approximately 9.7×10⁸ or more Tmem, approximately 9.8×10⁸ or more Tmem, approximately 9.9×10⁸ or more Tmem, approximately 1.0×10⁹ or more Tmem, approximately 1.1×10⁹ or more Tmem, approximately 1.2×10⁹ or more Tmem, approximately 1.3×10⁹ or more Tmem, approximately 1.4×10⁹ or more Tmem, approximately 1.5×10⁹ or more Tmem, approximately 1.6×10⁹ or more Tmem, approximately 1.7×10⁹ or more Tmem, approximately 1.8×10⁹ or more Tmem, approximately 1.9×10⁹ or more Tmem, approximately 2.0×10⁹ or more Tmem, approximately 2.1×10⁹ or more Tmem, approximately 2.2×10⁹ or more Tmem, approximately 2.3×10⁹ or more Tmem, approximately 2.4×10⁹ or more Tmem, approximately 2.5×10⁹ or more Tmem, approximately 2.6×10⁹ or more Tmem, approximately 2.7×10⁹ or more Tmem, approximately 2.8×10⁹ or more Tmem, approximately 2.9×10⁹ or more Tmem, approximately 3.0×10⁹ or more Tmem, approximately 3.1×10⁹ or more Tmem, approximately 3.2×10⁹ or more Tmem, approximately 3.3×10⁹ or more Tmem, approximately 3.4×10⁹ or more Tmem, approximately 3.5×10⁹ or more Tmem, approximately 3.6×10⁹ or more Tmem, approximately 3.7×10⁹ or more Tmem, approximately 3.8×10⁹ or more Tmem, approximately 3.9×10⁹ or more Tmem, approximately 4.0×10⁹ or more Tmem, approximately 4.1×10⁹ or more Tmem, approximately 4.2×10⁹ or more Tmem, approximately 4.3×10⁹ or more Tmem, approximately 4.4×10⁹ or more Tmem, approximately 4.5×10⁹ or more Tmem, approximately 4.6×10⁹ or more Tmem, approximately 4.7×10⁹ or more Tmem, approximately 4.8×10⁹ or more Tmem, approximately 4.9×10⁹ or more Tmem, approximately 5.0×10⁹ or more Tmem, approximately 5.1×10⁹ or more Tmem, approximately 5.2×10⁹ or more Tmem, approximately 5.3×10⁹ or more Tmem, approximately 5.4×10⁹ or more Tmem, approximately 5.5×10⁹ or more Tmem, approximately 5.6×10⁹ or more Tmem, approximately 5.7×10⁹ or more Tmem, approximately 5.8×10⁹ or more Tmem, approximately 5.9×10⁹ or more Tmem, approximately 6.0×10⁹ or more Tmem, approximately 6.1×10⁹ or more Tmem, approximately 6.2×10⁹ or more Tmem, approximately 6.3×10⁹ or more Tmem, approximately 6.4×10⁹ or more Tmem, approximately 6.5×10⁹ or more Tmem, approximately 6.6×10⁹ or more Tmem, approximately 6.7×10⁹ or more Tmem, approximately 6.8×10⁹ or more Tmem, approximately 6.9×10⁹ or more Tmem, approximately 7.0×10⁹ or more Tmem, approximately 7.1×10⁹ or more Tmem, approximately 7.2×10⁹ or more Tmem, approximately 7.3×10⁹ or more Tmem, approximately 7.4×10⁹ or more Tmem, approximately 7.5×10⁹ or more Tmem, approximately 7.6×10⁹ or more Tmem, approximately 7.7×10⁹ or more Tmem, approximately 7.8×10⁹ or more Tmem, approximately 7.9×10⁹ or more Tmem, approximately 8.0×10⁹ or more Tmem, approximately 8.1×10⁹ or more Tmem, approximately 8.2×10⁹ or more Tmem, approximately 8.3×10⁹ or more Tmem, approximately 8.4×10⁹ or more Tmem, approximately 8.5×10⁹ or more Tmem, approximately 8.6×10⁹ or more Tmem, approximately 8.7×10⁹ or more Tmem, approximately 8.8×10⁹ or more Tmem, approximately 8.9×10⁹ or more Tmem, approximately 9.0×10⁹ or more Tmem, approximately 9.1×10⁹ or more Tmem, approximately 9.2×10⁹ or more Tmem, approximately 9.3×10⁹ or more Tmem, approximately 9.4×10⁹ or more Tmem, approximately 9.5×10⁹ or more Tmem, approximately 9.6×10⁹ or more Tmem, approximately 9.7×10⁹ or more Tmem, approximately 9.8×10⁹ or more Tmem, approximately 9.9×10⁹ or more Tmem, approximately 1.0×10¹⁰ or more Tmem, approximately 1.1×10¹⁰ or more Tmem, approximately 1.2×10¹⁰ or more Tmem, approximately 1.3×10¹⁰ or more Tmem, approximately 1.4×10¹⁰ or more Tmem, approximately 1.5×10¹⁰ or more Tmem, approximately 1.6×10¹⁰ or more Tmem, approximately 1.7×10¹⁰ or more Tmem, approximately 1.8×10¹⁰ or more Tmem, approximately 1.9×10¹⁰ or more Tmem, approximately 2.0×10¹⁰ or more Tmem, approximately 2.1×10¹⁰ or more Tmem, approximately 2.2×10¹⁰ or more Tmem, approximately 2.3×10¹⁰ or more Tmem, approximately 2.4×10¹⁰ or more Tmem, approximately 2.5×10¹⁰ or more Tmem, approximately 2.6×10¹⁰ or more Tmem, approximately 2.7×10¹⁰ or more Tmem, approximately 2.8×10¹⁰ or more Tmem, approximately 2.9×10¹⁰ or more Tmem, approximately 3.0×10¹⁰ or more Tmem, approximately 3.1×10¹⁰ or more Tmem, approximately 3.2×10¹⁰ or more Tmem, approximately 3.3×10¹⁰ or more Tmem, approximately 3.4×10¹⁰ or more Tmem, approximately 3.5×10¹⁰ or more Tmem, approximately 3.6×10¹⁰ or more Tmem, approximately 3.7×10¹⁰ or more Tmem, approximately 3.8×10¹⁰ or more Tmem, approximately 3.9×10¹⁰ or more Tmem, approximately 4.0×10¹⁰ or more Tmem, approximately 4.1×10¹⁰ or more Tmem, approximately 4.2×10¹⁰ or more Tmem, approximately 4.3×10¹⁰ or more Tmem, approximately 4.4×10¹⁰ or more Tmem, approximately 4.5×10¹⁰ or more Tmem, approximately 4.6×10¹⁰ or more Tmem, approximately 4.7×10¹⁰ or more Tmem, approximately 4.8×110¹⁰ or more Tmem, approximately 4.9×10¹⁰ or more Tmem, or approximately 5.0×10¹⁰ or more Tmem (e.g., where Tmem are CD3+CD45RA− CD45RO+).

A population of cells of the present disclosure comprising Tmem, or comprising at least one dose of Tmem, can have a defined level of purity for Tmem cells. For example, a population of Tmem of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tmem cells as a percentage of total cells (e.g., where Tmem are CD3+CD45RA− CD45RO+).

A population of cells of the present disclosure comprising Tmem, or comprising at least one dose of Tmem, can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tmem as a percentage of total cells. (e.g., where Tmem are CD3+CD45RA− CD45RO+).

A population of cells of the present disclosure comprising Tmem, or comprising at least one dose of Tmem, can have a defined level of contaminating non-Tmem cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tmem cells per kg of recipient subject's actual body weight or ideal body weight are present in a population of Tmem of the disclosure (e.g., where the non-Tmem cells are CD45RO−).

In some embodiments, a population of cells of the present disclosure comprising Tmem, or comprising at least one dose of Tmem, comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tmem cells (e.g., where the non-Tmem cells are CD45RO−).

In some embodiments, a population of cells of the present disclosure comprising Tmem, or comprising at least one dose of Tmem, can comprise a population of T central memory cells (T_{CM}), a population of T effector memory cells (T_{EM}), a population of T stem central memory cells (T_{SCM}), or any combination thereof.

As used herein, “T central memory cell” or “T_{CM}” refers to one or more antigen experienced T cells that express the phenotypic markers CD4 or CD8, CD62L, CD45RO, CCR7, IL-2Rβ, CD28, CD127, and CD95 and do not express or have low expression of CD45RA as compared to naïve Tcon cells. Central memory T cells can differentiate into T_{EM} cells following antigen re-challenge.

In some embodiments, the population of cells of the present disclosure comprising Tmem, or comprising at least one dose of Tmem, can comprise a population of T central memory cells (T_{CM}), or at least one dose of T central memory cells (T_{CM}), comprising from approximately 1×10⁴ to approximately 1×10⁹ T_{CM} per kilogram of actual body weight or ideal body weight of a human subject receiving a treatment or cellular therapy product of the present disclosure. In some embodiments, the population of cells of the present disclosure comprising Tmem, or comprising at least one dose of Tmem, can comprise a population of T central memory cells (T_{CM}), or at least one dose of T central memory cells (T_{CM}), comprising a total dose of approximately 1.0×10⁵ to approximately 5.0×10¹⁰ T_{CM}.

A population of T_{CM}, or a dose of T_{CM}, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 1×10⁸, at least about 2×10⁸, at least about 3×10⁸, at least about 4×10⁷, at least about 5×10⁸, at least about 6×10⁸, at least about 7×10⁸, at least about 8×10⁸, at least about 9×10⁸, at least about 1×10⁹, or more T_{CM} per kilogram of actual body weight or ideal body weight of a human subject receiving a treatment or cellular therapy product of the present disclosure. In some embodiments, the T_{CM} are CD4⁺CD45RO⁺ or CD8⁺CD45RO⁺. In some embodiments, the T_{CM} are CD45RA⁻, CD62L⁺, CCR7⁺, or any combination thereof.

In some embodiments, a population of T_{CM}, or a dose of T_{CM}, can comprise approximately 1.0×10⁵ or more T_{CM}, approximately 2.0×10⁵ or more T_{CM}, approximately 3.0×10⁵ or more T_{CM}, approximately 4.0×10⁵ or more T_{CM}, approximately 5.0×10⁵ or more T_{CM}, approximately 6.0×10⁵ or more T_{CM}, approximately 7.0×10⁵ or more T_{CM}, approximately 8.0×10⁵ or more T_{CM}, approximately 9.0×10⁵ or more T_{CM}, approximately 1.0×10⁶ or more T_{CM}, approximately 2.0×10⁶ or more T_{CM}, approximately 3.0×10⁶ or more T_{CM}, approximately 4.0×10⁶ or more T_{CM}, approximately 5.0×10⁶ or more T_{CM}, approximately 6.0×10⁶ or more T_{CM}, approximately 7.0×10⁶ or more T_{CM}, approximately 8.0×10⁶ or more T_{CM}, approximately 9.0×10⁶ or more T_{CM}, approximately 1.0×10⁷ or more T_{CM}, approximately 2.0×10⁷ or more T_{CM}, approximately 3.0×10⁷ or more T_{CM}, approximately 4.0×10⁷ or more T_{CM}, approximately 5.0×10⁷ or more T_{CM}, approximately 6.0×10⁷ or more T_{CM}, approximately 7.0×10⁷ or more T_{CM}, approximately 8.0×10⁷ or more T_{CM}, approximately 9.0×10⁷ or more T_{CM}, approximately 1.0×10⁸ or more T_{CM}, approximately 2.0×10⁸ or more T_{CM}, approximately 3.0×10⁸ or more T_{CM}, approximately 4.0×10⁸ or more T_{CM}, approximately 5.0×10⁸ or more T_{CM}, approximately 6.0×10⁸ or more T_{CM}, approximately 7.0×10⁸ or more T_{CM}, approximately 8.0×10⁸ or more T_{CM}, approximately 9.0×10⁸ or more T_{CM}, approximately 1.0×10⁹ or more T_{CM}, approximately 2.0×10⁹ or more T_{CM}, approximately 3.0×10⁹ or more T_{CM}, approximately 4.0×10⁹ or more T_{CM}, approximately 5.0×10⁹ or more T_{CM}, approximately 6.0×10⁹ or more T_{CM}, approximately 7.0×10⁹ or more T_{CM}, approximately 8.0×10⁹ or more T_{CM}, approximately 9.0×10⁹ or more T_{CM}, approximately 1.0×10¹⁰ or more T_{CM}, approximately 2.0×10¹⁰ or more T_{CM}, approximately 3.0×10¹⁰ or more T_{CM}, approximately 4.0×10¹⁰ or more T_{CM}, or approximately 5.0×10¹⁰ or more T_{CM}. In some embodiments, the T_{CM} are CD4⁺CD45RO⁺ or CD8⁺CD45RO⁺. In some embodiments, the T_{CM} are CD45RA⁻, CD62L⁺, CCR7⁺, or any combination thereof.

As used herein, “T effector memory cell” or “T_{EM}” refers to one or more antigen experienced T cells that express the phenotypic markers CD4 or CD8, CD45RO, CD127, IL-2Rβ, and CD95, and do not express or have low expression of CD45RA, CD62L, CCR7, and CD28. T effector memory cells are terminally differentiated and acquire effector function after re-stimulation by antigen.

In some embodiments, the population of cells of the present disclosure comprising Tmem, or comprising at least one dose of Tmem, can comprise a population of T effector memory cells (T_{EM}), or at least one dose of T effector memory cells (T_{EM}), comprising from approximately 1×10⁴ to approximately 1×10⁹ T_{E} per kilogram of actual body weight or ideal body weight of a human subject receiving a treatment or cellular therapy product of the present disclosure. In some embodiments, the population of cells of the present disclosure comprising Tmem, or comprising at least one dose of Tmem, can comprise a population of T effector memory cells (T_{EM}), or at least one dose of T effector memory cells (T_{EM}), comprising a total dose of approximately 1.0×10⁵ to approximately 5.0×10¹⁰ T_{EM}.

A population of T_{EM}, or a dose of T_{EM}, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 1×10⁸, at least about 2×10⁸, at least about 3×10⁸, at least about 4×10⁷, at least about 5×10⁸, at least about 6×10⁸, at least about 7×10⁸, at least about 8×10⁸, at least about 9×10⁸, at least about 1×10⁹, or more T_{EM} per kilogram of actual body weight or ideal body weight of a human subject receiving a treatment or cellular therapy product of the present disclosure. In some embodiments, the T_{EM} are CD4⁺, CD45RO⁺, CD45RA⁻, CD62L⁻, CCR7⁻, or any combination thereof.

In some embodiments, a population of T_{EM}, or a dose of T_{EM}, can comprise approximately 1.0×10⁵ or more T_{EM}, approximately 2.0×10⁵ or more T_{EM}, approximately 3.0×10⁵ or more T_{EM}, approximately 4.0×10⁵ or more T_{EM}, approximately 5.0×10⁵ or more T_{EM}, approximately 6.0×10⁵ or more T_{EM}, approximately 7.0×10⁵ or more T_{EM}, approximately 8.0×10⁵ or more T_{EM}, approximately 9.0×10⁵ or more T_{EM}, approximately 1.0×10⁶ or more T_{EM}, approximately 2.0×10⁶ or more T_{EM}, approximately 3.0×10⁶ or more T_{EM}, approximately 4.0×10⁶ or more T_{EM}, approximately 5.0×10⁶ or more T_{EM}, approximately 6.0×10⁶ or more T_{EM}, approximately 7.0×10⁶ or more T_{EM}, approximately 8.0×10⁶ or more T_{EM}, approximately 9.0×10⁶ or more T_{EM}, approximately 1.0×10⁷ or more T_{EM}, approximately 2.0×10⁷ or more T_{EM}, approximately 3.0×10⁷ or more T_{EM}, approximately 4.0×10⁷ or more T_{EM}, approximately 5.0×10⁷ or more T_{EM}, approximately 6.0×10⁷ or more T_{EM}, approximately 7.0×10⁷ or more T_{EM}, approximately 8.0×10⁷ or more T_{EM}, approximately 9.0×10⁷ or more T_{EM}, approximately 1.0×10⁸ or more T_{EM}, approximately 2.0×10⁸ or more T_{EM}, approximately 3.0×10⁸ or more T_{EM}, approximately 4.0×10⁸ or more T_{EM}, approximately 5.0×10⁸ or more T_{EM}, approximately 6.0×10⁸ or more T_{EM}, approximately 7.0×10⁸ or more T_{EM}, approximately 8.0×10⁸ or more T_{EM}, approximately 9.0×10⁸ or more T_{EM}, approximately 1.0×10⁹ or more T_{EM}, approximately 2.0×10⁹ or more T_{EM}, approximately 3.0×10⁹ or more T_{EM}, approximately 4.0×10⁹ or more T_{EM}, approximately 5.0×10⁹ or more T_{EM}, approximately 6.0×10⁹ or more T_{EM}, approximately 7.0×10⁹ or more T_{EM}, approximately 8.0×10⁹ or more T_{EM}, approximately 9.0×10⁹ or more T_{EM}, approximately 1.0×10¹⁰ or more T_{EM}, approximately 2.0×10¹⁰ or more T_{EM}, approximately 3.0×10¹⁰ or more T_{EM}, approximately 4.0×10¹⁰ or more T_{EM}, or approximately 5.0×10¹⁰ or more T_{EM}. In some embodiments, the T_{EM} are CD4⁺, CD45RO⁺, CD45RA⁻, CD62L⁻, CCR7⁻, or any combination thereof.

As used herein, “T stem central memory cell” or “T_{SCM}” refers to one or more antigen experienced T cells that express the phenotypic markers CD4 or CD8, CD45RA, CD62L, CD95, IL-2Rβ, CCR7, CXCR3, CD122, and LFA-1. T_{SCM} cells possess memory T cell capability of rapid acquisition of effector function following antigen re-challenge, but have enhanced stem cell-like qualities such as long-term persistence compared to T_{CM} cells. T_{SCM} cells can generate central memory, effector memory, and effector T cell subsets.

In some embodiments, the population of cells of the present disclosure comprising Tmem, or comprising at least one dose of Tmem, can comprise a population of T stem central memory cells (T_{SCM}), or at least one dose of T stem central memory cells (T_{SCM}), comprising from approximately 1×10⁴ to approximately 1×10⁹ T_{E} per kilogram of actual body weight or ideal body weight of a human subject receiving a treatment or cellular therapy product of the present disclosure. In some embodiments, the population of cells of the present disclosure comprising Tmem, or comprising at least one dose of Tmem, can comprise a population of T stem central memory cells (T_{SCM}), or at least one dose of T stem central memory cells (T_{SCM}), comprising a total dose of approximately 1.0×10⁵ to approximately 5.0×10¹⁰ T_{SCM}.

A population of T_{SCM}, or a dose of T_{SCM}, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 1×10⁸, at least about 2×10⁸, at least about 3×10⁸, at least about 4×10⁷, at least about 5×10⁸, at least about 6×10⁸, at least about 7×10⁸, at least about 8×10⁸, at least about 9×10⁸, at least about 1×10⁹, or more T_{SCM} per kilogram of actual body weight or ideal body weight of a human subject receiving a treatment or cellular therapy product of the present disclosure. In some embodiments, the T_{SCM} are CD4⁺CD45RA⁺ or CD8⁺CD45RA⁺. In some embodiments, the T_{SCM} are CD95⁺, CD122⁺, CXCR3⁺, LFA-1+, or any combination thereof.

In some embodiments, a population of T_{SCM}, or a dose of T_{SCM}, can comprise approximately 1.0×10⁵ or more T_{SCM}, approximately 2.0×10⁵ or more T_{SCM}, approximately 3.0×10⁵ or more T_{SCM}, approximately 4.0×10⁵ or more T_{SCM}, approximately 5.0×10⁵ or more T_{SCM}, approximately 6.0×10⁵ or more T_{SCM}, approximately 7.0×10⁵ or more T_{SCM}, approximately 8.0×10⁵ or more T_{SCM}, approximately 9.0×10⁵ or more T_{SCM}, approximately 1.0×10⁶ or more T_{SCM}, approximately 2.0×10⁶ or more T_{SCM}, approximately 3.0×10⁶ or more T_{SCM}, approximately 4.0×10⁶ or more T_{SCM}, approximately 5.0×10⁶ or more T_{SCM}, approximately 6.0×10⁶ or more T_{SCM}, approximately 7.0×10⁶ or more T_{SCM}, approximately 8.0×10⁶ or more T_{SCM}, approximately 9.0×10⁶ or more T_{SCM}, approximately 1.0×10⁷ or more T_{SCM}, approximately 2.0×10⁷ or more T_{SCM}, approximately 3.0×10⁷ or more T_{SCM}, approximately 4.0×10⁷ or more T_{SCM}, approximately 5.0×10⁷ or more T_{SCM}, approximately 6.0×10⁷ or more T_{SCM}, approximately 7.0×10⁷ or more T_{SCM}, approximately 8.0×10⁷ or more T_{SCM}, approximately 9.0×10⁷ or more T_{SCM}, approximately 1.0×10⁸ or more T_{SCM}, approximately 2.0×10⁸ or more T_{SCM}, approximately 3.0×10⁸ or more T_{SCM}, approximately 4.0×10⁸ or more T_{SCM}, approximately 5.0×10⁸ or more T_{SCM}, approximately 6.0×10⁸ or more T_{SCM}, approximately 7.0×10⁸ or more T_{SCM}, approximately 8.0×10⁸ or more T_{SCM}, approximately 9.0×10⁸ or more T_{SCM}, approximately 1.0×10⁹ or more T_{SCM}, approximately 2.0×10⁹ or more T_{SCM}, approximately 3.0×10⁹ or more T_{SCM}, approximately 4.0×10⁹ or more T_{SCM}, approximately 5.0×10⁹ or more T_{SCM}, approximately 6.0×10⁹ or more T_{SCM}, approximately 7.0×10⁹ or more T_{SCM}, approximately 8.0×10⁹ or more T_{SCM}, approximately 9.0×10⁹ or more T_{SCM}, approximately 1.0×10¹⁰ or more T_{SCM}, approximately 2.0×10¹⁰ or more T_{SCM}, approximately 3.0×10¹⁰ or more T_{SCM}, approximately 4.0×10¹⁰ or more T_{SCM}, or approximately 5.0×10¹⁰ or more T_{SCM}. In some embodiments, the T_{SCM} are CD4⁺CD45RA⁺ or CD8⁺CD45RA⁺. In some embodiments, the T_{SCM} are CD95⁺, CD122⁺, CXCR3+, LFA-1+, or any combination thereof.

### Naïve Conventional T Cells (Tcon)

In some embodiments, a population of cells of the present disclosure can be depleted of naïve conventional αβ-T cells or naïve conventional CD3⁺CD25⁻CD45RA⁺ T cells (Tcon). In some embodiments, the population of cells depleted of Tcon comprises less than about 5×10⁵ Tcon per kilogram of actual body weight or ideal body weight of a human subject receiving a treatment or cellular therapy product of the present disclosure. In some embodiments, the population of cells depleted of Tcon comprises less than about 5×10⁵ Tcon per kilogram of IBW of the human subject.

In some embodiments, a population of cells depleted of naïve conventional αβ-T cells or naïve conventional CD3⁺CD25⁻CD45RA⁺ T cells (Tcon) can comprise less than approximately 5.0×10⁵, less than approximately 4.9×10⁵, less than approximately 4.8×10⁵, less than approximately 4.7×10⁵, less than approximately 4.6×10⁵, less than approximately 4.5×10⁵, less than approximately 4.4×10⁵, less than approximately 4.3×10⁵, less than approximately 4.2×10⁵, less than approximately 4.1×10⁵, less than approximately 4.0×10⁵, less than approximately 3.9×10⁵, less than approximately 3.8×10⁵, less than approximately 3.7×10⁵, less than approximately 3.6×10⁵, less than approximately 3.5×10⁵, less than approximately 3.4×10⁵, less than approximately 3.3×10⁵, less than approximately 3.2×10⁵, less than approximately 3.1×10⁵, less than approximately 3.0×10⁵, less than approximately 2.9×10⁵, less than approximately 2.8×10⁵, less than approximately 2.7×10⁵, less than approximately 2.6×10⁵, less than approximately 2.5×10⁵, less than approximately 2.4×10⁵, less than approximately 2.3×10⁵, less than approximately 2.2×10⁵, less than approximately 2.1×10⁵, less than approximately 2.0×10⁵, less than approximately 1.9×10⁵, less than approximately 1.8×10⁵, less than approximately 1.7×10⁵, less than approximately 1.6×10⁵, less than approximately 1.5×10⁵, less than approximately 1.4×10⁵, less than approximately 1.3×10⁵, less than approximately 1.2×10⁵, less than approximately 1.1×10⁵, less than approximately 1.0×10⁵, less than approximately 9.9×10⁴, less than approximately 9.8×10⁴, less than approximately 9.7×10⁴, less than approximately 9.6×10⁴, less than approximately 9.5×10⁴, less than approximately 9.4×10⁴, less than approximately 9.3×10⁴, less than approximately 9.2×10⁴, less than approximately 9.1×10⁴, less than approximately 9.0×10⁴, less than approximately 8.9×10⁴, less than approximately 8.8×10⁴, less than approximately 8.7×10⁴, less than approximately 8.6×10⁴, less than approximately 8.5×10⁴, less than approximately 8.4×10⁴, less than approximately 8.3×10⁴, less than approximately 8.2×10⁴, less than approximately 8.1×10⁴, less than approximately 8.0×10⁴, less than approximately 7.9×10⁴, less than approximately 7.8×10⁴, less than approximately 7.7×10⁴, less than approximately 7.6×10⁴, less than approximately 7.5×10⁴, less than approximately 7.4×10⁴, less than approximately 7.3×10⁴, less than approximately 7.2×10⁴, less than approximately 7.1×10⁴, less than approximately 7.0×10⁴, less than approximately 6.9×10⁴, less than approximately 6.8×10⁴, less than approximately 6.7×10⁴, less than approximately 6.6×10⁴, less than approximately 6.5×10⁴, less than approximately 6.4×10⁴, less than approximately 6.3×10⁴, less than approximately 6.2×10⁴, less than approximately 6.1×10⁴, less than approximately 6.0×10⁴, less than approximately 5.9×10⁴, less than approximately 5.8×10⁴, less than approximately 5.7×10⁴, less than approximately 5.6×10⁴, less than approximately 5.5×10⁴, less than approximately 5.4×10⁴, less than approximately 5.3×10⁴, less than approximately 5.2×10⁴, less than approximately 5.1×10⁴, less than approximately 5.0×10⁴, less than approximately 4.9×10⁴, less than approximately 4.8×10⁴, less than approximately 4.7×10⁴, less than approximately 4.6×10⁴, less than approximately 4.5×10⁴, less than approximately 4.4×10⁴, less than approximately 4.3×10⁴, less than approximately 4.2×10⁴, less than approximately 4.1×10⁴, less than approximately 4.0×10⁴, less than approximately 3.9×10⁴, less than approximately 3.8×10⁴, less than approximately 3.7×10⁴, less than approximately 3.6×10⁴, less than approximately 3.5×10⁴, less than approximately 3.4×10⁴, less than approximately 3.3×10⁴, less than approximately 3.2×10⁴, less than approximately 3.1×10⁴, less than approximately 3.0×10⁴, less than approximately 2.9×10⁴, less than approximately 2.8×10⁴, less than approximately 2.7×10⁴, less than approximately 2.6×10⁴, less than approximately 2.5×10⁴, less than approximately 2.4×10⁴, less than approximately 2.3×10⁴, less than approximately 2.2×10⁴, less than approximately 2.1×10⁴, less than approximately 2.0×10⁴, less than approximately 1.9×10⁴, less than approximately 1.8×10⁴, less than approximately 1.7×10⁴, less than approximately 1.6×10⁴, less than approximately 1.5×10⁴, less than approximately 1.4×10⁴, less than approximately 1.3×10⁴, less than approximately 1.2×10⁴, less than approximately 1.1×10⁴, less than approximately 1.0×10⁴, less than approximately 9.9×10³, less than approximately 9.8×10³, less than approximately 9.7×10³, less than approximately 9.6×10³, less than approximately 9.5×10³, less than approximately 9.4×10³, less than approximately 9.3×10³, less than approximately 9.2×10³, less than approximately 9.1×10³, less than approximately 9.0×10³, less than approximately 8.9×10³, less than approximately 8.8×10³, less than approximately 8.7×10³, less than approximately 8.6×10³, less than approximately 8.5×10³, less than approximately 8.4×10³, less than approximately 8.3×10³, less than approximately 8.2×10³, less than approximately 8.1×10³, less than approximately 8.0×10³, less than approximately 7.9×10³, less than approximately 7.8×10³, less than approximately 7.7×10³, less than approximately 7.6×10³, less than approximately 7.5×10³, less than approximately 7.4×10³, less than approximately 7.3×10³, less than approximately 7.2×10³, less than approximately 7.1×10³, less than approximately 7.0×10³, less than approximately 6.9×10³, less than approximately 6.8×10³, less than approximately 6.7×10³, less than approximately 6.6×10³, less than approximately 6.5×10³, less than approximately 6.4×10³, less than approximately 6.3×10³, less than approximately 6.2×10³, less than approximately 6.1×10³, less than approximately 6.0×10³, less than approximately 5.9×10³, less than approximately 5.8×10³, less than approximately 5.7×10³, less than approximately 5.6×10³, less than approximately 5.5×10³, less than approximately 5.4×10³, less than approximately 5.3×10³, less than approximately 5.2×10³, less than approximately 5.1×10³, less than approximately 5.0×10³, less than approximately 4.9×10³, less than approximately 4.8×10³, less than approximately 4.7×10³, less than approximately 4.6×10³, less than approximately 4.5×10³, less than approximately 4.4×10³, less than approximately 4.3×10³, less than approximately 4.2×10³, less than approximately 4.1×10³, less than approximately 4.0×10³, less than approximately 3.9×10³, less than approximately 3.8×10³, less than approximately 3.7×10³, less than approximately 3.6×10³, less than approximately 3.5×10³, less than approximately 3.4×10³, less than approximately 3.3×10³, less than approximately 3.2×10³, less than approximately 3.1×10³, less than approximately 3.0×10³, less than approximately 2.9×10³, less than approximately 2.8×10³, less than approximately 2.7×10³, less than approximately 2.6×10³, less than approximately 2.5×10³, less than approximately 2.4×10³, less than approximately 2.3×10³, less than approximately 2.2×10³, less than approximately 2.1×10³, less than approximately 2.0×10³, less than approximately 1.9×10³, less than approximately 1.8×10³, less than approximately 1.7×10³, less than approximately 1.6×10³, less than approximately 1.5×10³, less than approximately 1.4×10³, less than approximately 1.3×10³, less than approximately 1.2×10³, less than approximately 1.1×10³, or less than approximately 1.0×10³ Tcon per kg of the human subject's actual body weight or (adjusted) ideal body weight actual body weight or (adjusted) ideal body weight.

In some embodiments, a population of cells depleted of naïve conventional αβ-T cells or naïve conventional CD3⁺CD25⁻CD45RA T cells (Tcon) can comprise about 0 to about 100,000 Tcon per kg of the human subject's actual body weight or (adjusted) ideal body weight actual body weight or (adjusted) ideal body weight. In some embodiments, a population of cells depleted of naïve conventional αβ-T cells or naïve conventional CD3⁺CD25⁻CD45RA⁺ T cells (Tcon) can comprise about 0 to about 5,000, about 0 to about 10,000, about 0 to about 20,000, about 0 to about 30,000, about 0 to about 40,000, about 0 to about 50,000, about 0 to about 60,000, about 0 to about 70,000, about 0 to about 80,000, about 0 to about 90,000, about 0 to about 100,000, about 5,000 to about 10,000, about 5,000 to about 20,000, about 5,000 to about 30,000, about 5,000 to about 40,000, about 5,000 to about 50,000, about 5,000 to about 60,000, about 5,000 to about 70,000, about 5,000 to about 80,000, about 5,000 to about 90,000, about 5,000 to about 100,000, about 10,000 to about 20,000, about 10,000 to about 30,000, about 10,000 to about 40,000, about 10,000 to about 50,000, about 10,000 to about 60,000, about 10,000 to about 70,000, about 10,000 to about 80,000, about 10,000 to about 90,000, about 10,000 to about 100,000, about 20,000 to about 30,000, about 20,000 to about 40,000, about 20,000 to about 45,000, about 20,000 to about 50,000, about 20,000 to about 55,000, about 20,000 to about 60,000, about 20,000 to about 70,000, about 20,000 to about 75,000, about 20,000 to about 80,000, about 20,000 to about 90,000, about 20,000 to about 100,000, about 30,000 to about 40,000, about 30,000 to about 45,000, about 30,000 to about 50,000, about 30,000 to about 55,000, about 30,000 to about 60,000, about 30,000 to about 70,000, about 30,000 to about 75,000, about 30,000 to about 80,000, about 30,000 to about 90,000, about 30,000 to about 100,000, about 40,000 to about 45,000, about 40,000 to about 50,000, about 40,000 to about 55,000, about 40,000 to about 60,000, about 40,000 to about 70,000, about 40,000 to about 75,000, about 40,000 to about 80,000, about 40,000 to about 90,000, about 40,000 to about 100,000, about 50,000 to about 60,000, about 50,000 to about 65,000 about 50,000 to about 70,000, about 50,000 to about 75,000 about 50,000 to about 80,000, about 50,000 to about 90,000, about 50,000 to about 100,000, about 60,000 to about 70,000, 60,000 to about 75,000 about 60,000 to about 80,000, about 60,000 to about 90,000, about 60,000 to about 100,000, about 70,000 to about 80,000, about 70,000 to about 75,000 about 70,000 to about 90,000, about 70,000 to about 100,000, about 75,000 to about 80,000, about 75,000 to about 90,000, about 75,000 to about 100,000, about 80,000 to about 90,000, about 80,000 to about 100,000, or about 90,000 to about 100,000 Tcon per kg of per kg of the human subject's actual body weight or (adjusted) ideal body weight actual body weight or (adjusted) ideal body weight.

In some embodiments, a population of cells depleted of naïve conventional αβ-T cells or naïve conventional CD3⁺CD25⁻CD45RA⁺ T cells (Tcon) can comprise about 0, about 2500, about 5,000, about 10,000, about 15,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, or about 100,000 Tcon per kg of per kg of the human subject's actual body weight or (adjusted) ideal body weight actual body weight or (adjusted) ideal body weight.

In some embodiments, a population of cells depleted of naïve conventional αβ-T cells or naïve conventional CD3⁺CD25⁻CD45RA⁺ T cells (Tcon) can comprise at least about 0, at least about 5,000, at least about 10,000, at least about 15,000, at least about 20,000, at least about 25,000, at least about 30,000, at least about 35,000, at least about 40,000, at least about 45,000, at least about 50,000, at least about 55,000, at least about 60,000, at least about 65,000, at least about 70,000, at least about 75,000, at least about 80,000, at least about 85,000, or at least about 90,000 Tcon per kg of per kg of the human subject's actual body weight or (adjusted) ideal body weight actual body weight or (adjusted) ideal body weight.

In some embodiments, a population of cells depleted of naïve conventional αβ-T cells or naïve conventional CD3⁺CD25⁻CD45RA⁺ T cells (Tcon) can comprise at most about 5,000, at most about 10,000, at most about 15,000, at most about 20,000, at most about 25,000, at most about 30,000, at most about 35,000, at most about 40,000, at most about 45,000, at most about 50,000, at most about 55,000, at most about 60,000, at most about 65,000, at most about 70,000, at most about 75,000, at most about 80,000, at most about 85,000, at most about 90,000, at most about 95,000, or at most about 100,000 Tcon per kg of per kg of the human subject's actual body weight or (adjusted) ideal body weight actual body weight or (adjusted) ideal body weight.

In some embodiments, a population of cells depleted of naïve conventional αβ-T cells or naïve conventional CD3⁺CD25⁻CD45RA T cells (Tcon) can comprise less than approximately 6.0×10⁷, less than approximately 5.9×10⁷, less than approximately 5.8×10⁷, less than approximately 5.7×10⁷, less than approximately 5.6×10⁷, less than approximately 5.5×10⁷, less than approximately 5.4×10⁷, less than approximately 5.3×10⁷, less than approximately 5.2×10⁷, less than approximately 5.1×10⁷, less than approximately 5.0×10⁷, less than approximately 4.9×10⁷, less than approximately 4.8×10⁷, less than approximately 4.7×10⁷, less than approximately 4.6×10⁷, less than approximately 4.5×10⁷, less than approximately 4.4×10⁷, less than approximately 4.3×10⁷, less than approximately 4.2×10⁷, less than approximately 4.1×10⁷, less than approximately 4.0×10⁷, less than approximately 3.9×10⁷, less than approximately 3.8×10⁷, less than approximately 3.7×10⁷, less than approximately 3.6×10⁷, less than approximately 3.5×10⁷, less than approximately 3.4×10⁷, less than approximately 3.3×10⁷, less than approximately 3.2×10⁷, less than approximately 3.1×10⁷, less than approximately 3.0×10⁷, less than approximately 2.9×10⁷, less than approximately 2.8×10⁷, less than approximately 2.7×10⁷, less than approximately 2.6×10⁷, less than approximately 2.5×10⁷, less than approximately 2.4×10⁷, less than approximately 2.3×10⁷, less than approximately 2.2×10⁷, less than approximately 2.1×10⁷, less than approximately 2.0×10⁷, less than approximately 1.9×10⁷, less than approximately 1.8×10⁷, less than approximately 1.7×10⁷, less than approximately 1.6×10⁷, less than approximately 1.5×10⁷, less than approximately 1.4×10⁷, less than approximately 1.3×10⁷, less than approximately 1.2×10⁷, less than approximately 1.1×10⁷, less than approximately 1.0×10⁷, less than approximately 9.9×10⁶, less than approximately 9.8×10⁶, less than approximately 9.7×10⁶, less than approximately 9.6×10⁶, less than approximately 9.5×10⁶, less than approximately 9.4×10⁶, less than approximately 9.3×10⁶, less than approximately 9.2×10⁶, less than approximately 9.1×10⁶, less than approximately 9.0×10⁶, less than approximately 8.9×10⁶, less than approximately 8.8×10⁶, less than approximately 8.7×10⁶, less than approximately 8.6×10⁶, less than approximately 8.5×10⁶, less than approximately 8.4×10⁶, less than approximately 8.3×10⁶, less than approximately 8.2×10⁶, less than approximately 8.1×10⁶, less than approximately 8.0×10⁶, less than approximately 7.9×10⁶, less than approximately 7.8×10⁶, less than approximately 7.7×10⁶, less than approximately 7.6×10⁶, less than approximately 7.5×10⁶, less than approximately 7.4×10⁶, less than approximately 7.3×10⁶, less than approximately 7.2×10⁶, less than approximately 7.1×10⁶, less than approximately 7.0×10⁶, less than approximately 6.9×10⁶, less than approximately 6.8×10⁶, less than approximately 6.7×10⁶, less than approximately 6.6×10⁶, less than approximately 6.5×10⁶, less than approximately 6.4×10⁶, less than approximately 6.3×10⁶, less than approximately 6.2×10⁶, less than approximately 6.1×10⁶, less than approximately 6.0×10⁶, less than approximately 5.9×10⁶, less than approximately 5.8×10⁶, less than approximately 5.7×10⁶, less than approximately 5.6×10⁶, less than approximately 5.5×10⁶, less than approximately 5.4×10⁶, less than approximately 5.3×10⁶, less than approximately 5.2×10⁶, less than approximately 5.1×10⁶, less than approximately 5.0×10⁶, less than approximately 4.9×10⁶, less than approximately 4.8×10⁶, less than approximately 4.7×10⁶, less than approximately 4.6×10⁶, less than approximately 4.5×10⁶, less than approximately 4.4×10⁶, less than approximately 4.3×10⁶, less than approximately 4.2×10⁶, less than approximately 4.1×10⁶, less than approximately 4.0×10⁶, less than approximately 3.9×10⁶, less than approximately 3.8×10⁶, less than approximately 3.7×10⁶, less than approximately 3.6×10⁶, less than approximately 3.5×10⁶, less than approximately 3.4×10⁶, less than approximately 3.3×10⁶, less than approximately 3.2×10⁶, less than approximately 3.1×10⁶, less than approximately 3.0×10⁶, less than approximately 2.9×10⁶, less than approximately 2.8×10⁶, less than approximately 2.7×10⁶, less than approximately 2.6×10⁶, less than approximately 2.5×10⁶, less than approximately 2.4×10⁶, less than approximately 2.3×10⁶, less than approximately 2.2×10⁶, less than approximately 2.1×10⁶, less than approximately 2.0×10⁶, less than approximately 1.9×10⁶, less than approximately 1.8×10⁶, less than approximately 1.7×10⁶, less than approximately 1.6×10⁶, less than approximately 1.5×10⁶, less than approximately 1.4×10⁶, less than approximately 1.3×10⁶, less than approximately 1.2×10⁶, less than approximately 1.1×10⁶, or less than approximately 1.0×10⁶ Tcon.

In some embodiments, the number of naïve conventional αβ-T cells or naïve conventional CD3⁺CD25⁻CD45RA⁺ T cells (Tcon) in a cell population of the present disclosure (e.g., a population of isolated CD45⁺ cells) or composition of the present disclosure (e.g., a cellular therapy product) can be less than about 2%, 5%, 7%, 10%, 12%, 15%, 17%, 20%, 25%, 27%, 30%, 32% or 35% of the number of HSPC. In some embodiments, the number of Tcon can be about 2%, 5%, 7%, 1%, 12%, 15%, 17%, 20%, 25%, 27%, 30%, 32% or 35% of the number of HSPC. In some embodiments, the number of Tcon can be about 2% to about 7%, about 2% to about 10%, about 2% to about 15%, about 2% to about 20%, about 2% to about 25%, about 2% to about 30%, about 2% to about 35%, about 7% to about 10%, about 7% to about 15%, about 7% to about 20%, about 7% to about 25%, about 7% to about 30%, about 7% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35% of the number of HSPC.

In some embodiments, the number of naïve conventional αβ-T cells or naïve conventional CD3⁺CD25⁻CD45RA⁺ T cells (Tcon) in a cell population of the present disclosure (e.g., a population of isolated CD45⁺ cells) or composition of the present disclosure (e.g., a cellular therapy product) can be less than about 0.1%, 1%, 2%, 5%, 7%, 10%, 12%, 15%, 17%, 20%, 22% or 25% of the number of Tmem. In some embodiments, the number of Tcon can be about 0.1% to about 2%, about 0.1% to about 5%, about 0.1% to about 10%, about 0.1% to about 15%, about 0.1% to about 20%, about 2% to about 5%, about 2% to about 10%, about 2% to about 15%, about 2% to about 20%, about 5% to about 10%, about 5% to about 15%, about 5% to about 20%, about 10% to about 15%, about 10% to about 20%, or about 15% to about 20% of the number of Tmem.

In some embodiments, the number of naïve conventional αβ-T cells or naïve conventional CD3⁺CD25⁻CD45RA⁺ T cells (Tcon) in a cell population of the present disclosure (e.g., a population of isolated CD45⁺ cells) or composition of the present disclosure (e.g., a cellular therapy product) can be less than about 0.05%, 0.5%, 1%, 2%, 5%, 7%, 10%, 12%, 15%, 17%, 20%, 22%, 25% or 30% of the number of Treg. In some embodiments, the number of Tcon can be about 0.05% to about 1%, about 0.05% to about 5%, about 0.05% to about 10%, about 0.05% to about 15%, about 0.05% to about 20%, about 0.05% to about 30%, about 1% to about 5%, about 1% to about 10%, about 1% to about 15%, about 1% to about 20%, about 1% to about 30%, about 5% to about 10%, about 5% to about 15%, about 5% to about 20%, about 5% to about 30%, about 10% to about 15%, about 10% to about 20%, about 10% to about 30%, about 15% to about 20%, about 15% to about 30%, or about 20% to about 30% of the number of Treg.

In some embodiments, the number of naïve conventional αβ-T cells or naïve conventional CD3⁺CD25⁻CD45RA⁺ T cells (Tcon) in a cell population of the present disclosure (e.g., a population of isolated CD45⁺ cells) or composition of the present disclosure (e.g., a cellular therapy product) can be less than about 1%, 10%, 20%, 50%, 70%, 80% or 90% of the number of iNKT. In some embodiments, the number of Tcon can be about 1% to about 10%, about 1% to about 20%, about 1% to about 50%, about 1% to about 70%, about 1% to about 80%, about 1% to about 90%, about 10% to about 20%, about 10% to about 50%, about 10% to about 70%, about 10% to about 80%, about 10% to about 90%, about 20% to about 50%, about 20% to about 70%, about 20% to about 80%, about 20% to about 90%, about 50% to about 70%, about 50% to about 80%, about 50% to about 90%, about 70% to about 80%, about 70% to about 90%, or about 80% to about 90% of the number of iNKT.

### Cell Ratios

In certain embodiments, a population of cells of the present disclosure (e.g., a population of isolated CD45⁺ cells) can comprise a ratio of HSPC to another cell type. In some embodiments, the ratio of HSPC to Tmem comprises a range from 500:1 to 1:1,000, 400:1 to 1:1,000, 300:1 to 1:1,000, 200:1 to 1:1,000, 100:1 to 1:1,000, 50:1 to 1:1,000, 10:1 to 1:1,000, 5:1 to 1:1,000, 4:1 to 1:1,000, 3:1 to 1:1,000, 2:1 to 1:1,000, 1:1 to 1:1,000, 500:1 to 1:900, 500:1 to 1:800, 500:1 to 1:700, 500:1 to 1:600, 500:1 to 1:500, 500:1 to 1:400, 500:1 to 1:300, 500:1 to 1:200, 500:1 to 1:100, 500:1 to 1:50, 500:1 to 1:20, 500:1 to 1:10, 500:1 to 1:9, 500:1 to 1:8, 500:1 to 1:7, 500:1 to 1:6, 500:1 to 1:5, 500:1 to 1:4, 500:1 to 1:3, 500:1 to 1:2, 500:1 to 1:1, 400:1 to 1:900, 300:1 to 1:800, 200:1 to 1:700, 100:1 to 1:600, 50:1 to 1:500, 10:1 to 1:400, 5:1 to 1:300, 4:1 to 1:200, 3:1 to 1:100, 2:1 to 1:50, or 1:1 to 1:20. In some embodiments, the ratio of HSPC to Tmem comprises a range from 10:1 to 1:200, 100:1 to 1:2,000, or 1,000:1 to 1:20,000. The ratio of HSPC to Treg can comprise a range from 20:1 to 1:3, 100:1 to 1:30, or 200:1 to 1:300. The ratio of HSPC to naïve Treg can comprise a range from 1:500 to 100:1, 1:400 to 100:1, 1:300 to 100:1, 1:200 to 100:1, 1:100 to 100:1, 1:50 to 100:1, 1:20 to 100:1, 1:10 to 100:1, 1:5 to 100:1, 1:1 to 100:1, 1:200 to 50:1, 1:200 to 20:1, 1:200 to 10:1, 1:200 to 5:1, 1:100 to 1:1, 40:1 to 1:3, 200:1 to 1:15, or 400:1 to 1:150. The ratio of HSPC to Memory Treg can comprise a range from 1:500 to 10,000:1, 1:400 to 10,000:1, 1:300 to 10,000:1, 1:200 to 10,000:1, 1:100 to 10,000:1, 1:50 to 10,000:1, 1:20 to 10,000:1, 1:10 to 10,000:1, 1:5 to 10,000:1, 1:1 to 10,000:1, 1:500 to 5,000:1, 1:500 to 1,000:1, 1:500 to 900:1, 1:500 to 800:1, 1:500 to 700:1, 1:500 to 600:1, 1:500 to 500:1, 1:500 to 400:1, 1:500 to 300:1, 1:500 to 200:1, 1:500 to 100:1, 1:500 to 50:1, 1:500 to 20:1, 1:500 to 10:1, 1:500 to 5:1, or 1:500 to 1:1. The ratio of HSPC to iNKT can comprise a range from 1:2 to 1,000,000:1, 1:2 to 500,000:1, 1:1 to 500,000:1, 100:1 to 1,000,000:1, 100:1 to 500,000:1, 100:1 to 100,000:1, 500:1 to 1,000,000:1, 500:1 to 500,000:1, 500:1 to 100,000:1, 1,000:1 to 100,000:1, 1,000:1 to 1,000,000:1, 1,000:1 to 500,000:1, 1,000:1 to 100,000:1, 10,000:1 to 1:2, 100,000:1-1:20, or 1,000,000:1-1:200.

In certain embodiments, a population of cells of the present disclosure (e.g., a population of isolated CD45⁺ cells) can comprise a ratio of naïve conventional αβ-T cells, or naïve conventional CD3⁺CD25⁻CD45RA⁺ T cells, (Tcon) to a therapeutic cell type. The ratio of Tcon to HSPC can be less than 1:3, less than 1:50, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:3,000, less than 1:4,000, less than 1:5,000, less than 1:6,000, less than 1:7,000, less than 1:8,000, less than 1:9,000, less than 1:10,000, less than 1:50,000, less than 1:100,000, less than 1:200,000, less than 1:300,000, less than 1:400,000, less than 1:500,000, less than 1:600,000, less than 1:700,000, less than 1:800,000, less than 1:900,000, less than 1:1,000,000. The ratio of Tcon to Tmem can be less than 1:30, less than 1:200, less than 1:300, less than 1:400, less than 1:500, less than 1:600, less than 1:700, less than 1:800, less than 1:900, less than 1:1000, less than 1:5000, less than 1:10000, less than 1:15000, less than 1:20000, less than 1:25000, less than 1:30000, less than 1:35000, less than 1:40000, less than 1:45000, or less than 1:50000. The ratio of Tcon to Treg can be less than 1:1, less than 1:2, less than 1:3, less than 1:4, less than 1:5, less than 1:6, less than 1:7, less than 1:8, less than 1:9, 1:10, less than 1:15, less than 1:20, less than 1:30, less than 1:200, less than 1:300, less than 1:400, less than 1:500, less than 1:600, less than 1:700, less than 1:800, less than 1:900, less than 1:1000, less than 1:5000, less than 1:10000, less than 1:15000, less than 1:20000, less than 1:25000, less than 1:30000, less than 1:35000, less than 1:40000, less than 1:45000, or less than 1:50000. The ratio of Tcon to naïve Treg cells can be less than 1:1, less than 1:2, less than 1:3, less than 1:4, less than 1:5, less than 1:6, less than 1:7, less than 1:8, less than 1:9, 1:10, less than 1:15, less than 1:20, less than 1:30, less than 1:200, less than 1:300, less than 1:400, less than 1:500, less than 1:600, less than 1:700, less than 1:800, less than 1:900, less than 1:1000, less than 1:5000, less than 1:10000, less than 1:15000, less than 1:20000, less than 1:25000, less than 1:30000, less than 1:35000, less than 1:40000, less than 1:45000, or less than 1:50000. The ratio of Tcon to memory Treg cells can be less than 1:1, less than 1:2, less than 1:3, less than 1:4, less than 1:5, less than 1:6, less than 1:7, less than 1:8, less than 1:9, less than 1:10, less than 1:15, less than 1:20, less than 1:30, less than 1:200, less than 1:300, less than 1:400, less than 1:500, less than 1:600, less than 1:700, less than 1:800, less than 1:900, less than 1:1000, less than 1:5000, less than 1:10000, less than 1:15000, less than 1:20000, less than 1:25000, less than 1:30000, less than 1:35000, less than 1:40000, less than 1:45000, or less than 1:50000. The ratio of Tcon to iNKT can be less than 100:1, less than 1:1, less than 1:2, less than 1:3, less than 1:4, less than 1:5, less than 1:6, less than 1:7, less than 1:8, less than 1:9, less than 1:10, less than 1:15, less than 1:20, less than 1:30, less than 1:200, less than 1:300, less than 1:400, less than 1:500, less than 1:600, less than 1:700, less than 1:800, less than 1:900, less than 1:1000, less than 1:5000, less than 1:10000, less than 1:15000, less than 1:20000, less than 1:25000, less than 1:30000, less than 1:35000, less than 1:40000, less than 1:45000, less than 1:50000.

In some embodiments, a population of cells of the present disclosure (e.g., a population of isolated CD45⁺ cells) can comprise a ratio of Tmem to Treg that is not dependent on the concentration of starting cells (e.g., substantially modified from the starting concentrations). This can provide an advantage because the concentration of Tmem can be controlled (e.g., dose escalated) independent of the concentration of Treg. The ratio of Tmem to Treg can be from 30:1 to 1:1, 25:1 to 1:1, 20:1 to 1:1, 15:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, 2:1 to 1:1. In certain embodiments, the ratio of Tmem to Treg can be from 1:1 to 200:1, 1:10 to 2000:1, or 1:100 to 20,000:1. The ratio of Tmem to naïve Treg cells can be from 5:1 to 1:10, 3:1 to 1:10, 3:1 to 1:10, 2:1 to 1:10, or 1:1 to 1:10. The ratio of Tmem to memory Treg cells can be from 27:1 to 0.9:1, 30:1 to 1:10, 25:1 to 1:10, 20:1 to 1:10, 15:1 to 1:10, 10:1 to 1:10, 30:1 to 1:9, 30:1 to 1:8, 30:1 to 1:7, 30:1 to 1:6, 30:1 to 1:5, 30:1 to 1:4, 30:1 to 1:3, 30:1 to 1:2, or 30:1 to 1:1.

In some embodiments, a population of cells of the present disclosure (e.g., a population of isolated CD45⁺ cells) can comprise a ratio of Treg to iNKT that can be from 20,000:1 to 1:5, 200,000:1 to 1:50, or 2,000,000:1 to 1:500. In some embodiments, a population of cells of the present disclosure (e.g., a population of isolated CD45⁺ cells) can comprise a ratio of iNKT to Tmem can be from 2:1 to 1:100,000, 5:1 to 1:1,000,000, or 10:1 to 1:10,000,000.

### Administration of Cell Populations

Provided herein is a pharmaceutical treatment, pharmaceutical composition, cellular therapy, and/or cellular therapy product to be administered to a subject (e.g., a human subject) in need thereof. In some embodiments, the treatment, composition, therapy, or product may comprise a population of cells (e.g., a population of isolated CD45⁺ cells) comprising an enriched population of hematopoietic stem/progenitor cells (HSPC), memory T cells (Tmem), and regulatory T cells (Treg), wherein the population of cells is depleted of naïve conventional αβ-T cells or naïve conventional CD3⁺CD25⁻CD45RA⁺ T cells (Tcon). In some embodiments, the treatment, composition, therapy, or product may further include a solution comprising one or more doses of a graft vs host disease (GVHD) prophylactic agent (e.g., tacrolimus).

Another aspect provided herein relates to methods of treating a subject (e.g., a human subject) having or suspected of having a hematologic malignancy (e.g., a leukemia, lymphoma, myelodysplastic syndrome, or myelofibrosis) or an autoimmune disorder (e.g., multiple sclerosis). In some embodiments, a method of the present disclosure can comprise administering to a subject (e.g., a human subject) a solution comprising an enriched population of hematopoietic stem/progenitor cells (HSPC), memory T cells (Tmem), and regulatory T cells (Treg), wherein the population of cells is depleted of naïve conventional αβ-T cells or naïve conventional CD3⁺CD25⁻CD45RA⁺ T cells (Tcon). In other embodiments, a method of the present disclosure can comprise administering to a subject (e.g., a human subject) a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim}regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). In some embodiments, the methods of the present disclosure further comprise administering a solution comprising one or more doses of a graft vs host disease (GVHD) prophylactic agent (e.g., tacrolimus). In this aspect, the solution provided herein is as defined according to any herein disclosed pharmaceutical treatment, pharmaceutical composition, cellular therapy, and/or cellular therapy product.

Provided herein are methods for enhanced allogeneic hematopoietic stem cell transplantation, comprising administering to a subject solutions that comprise populations of cells. In some embodiments, the populations of cells comprise an enriched population of hematopoietic stem/progenitor cells (HSPC), memory T cells (Tmem), and regulatory T cells (Treg), wherein the population of cells is depleted of naïve conventional αβ-T cells or naïve conventional CD3⁺CD25⁻CD45RA⁺ T cells (Tcon). In some embodiments, the populations of cells are administered at the same time or at similar times, or at different times to the human subject. In some embodiments, the populations of cells are administered in one or more doses comprising the cell populations. In some embodiments, the populations of cells are administered in 2 doses comprising the cell populations. In some embodiments, the populations of cells are administered in 3 doses comprising the cell populations.

In some embodiments, after administration of an initial dose of the populations of cells, subsequent doses can be administered as soon as possible on the same day of the administration of the initial dose, or on the following day of the administration of the initial dose. In some embodiments, subsequent doses of the same cell populations can be administered to the subject within 24 hours of the initial dose. In some embodiments, subsequent doses of the same cell population can be administered to the subject at least about 2, 4, 6, 8, 10, 12, 14, 16, 18, 20, 22, or 24 hours after the initial population of cells.

### GVHD Prophylactic Agents

In some embodiments, subjects administered a composition of the present disclosure (e.g., a cellular therapy product or population of isolate CD45⁺ cells) may also be administered one or more GVHD prophylactic agents. In some embodiments, subjects administered a composition of the present disclosure (e.g., a cellular therapy product or population of isolated CD45⁺ cells) may also be administered a single GVHD prophylactic agent. In some embodiments, subjects administered, e.g., a single GVHD prophylactic agent exhibit a low incidence of ≥grade 1 aGVHD, for example, a lower incidence of ≥grade 1 aGVHD than subjects that are administered an alternate composition. In some embodiments, subjects administered a composition of the disclosure (e.g., a cellular therapy product or population of isolated CD45⁺ cells) without a GVHD prophylactic agent exhibit a low incidence of ≥grade 1 aGVHD, for example, a lower incidence of ≥grade 1 aGVHD than subjects that are administered an alternate composition.

A GVHD prophylactic agent of the present disclosure can be a calcineurin inhibitor, such as tacrolimus, cyclosporine, voclosporin, etc., or another agent which acts on the same targets as tacrolimus or comprises an active fragment of tacrolimus. In some embodiments, a GVHD prophylactic agent of the present disclosure can be an mTOR inhibitor, such as sirolimus, everolimus, zotarolimus, etc.

In some embodiments, a low dose of a GVHD prophylactic agent is tacrolimus with a target trough level (e.g., trough blood level) of approximately 5 ng/mL to approximately 10 ng/mL. In some embodiments, a low dose of a GVHD prophylactic agent is tacrolimus with a target trough level of about 4 ng/mL to about 6 ng/mL. In some embodiments, a low dose of a GVHD prophylactic agent is tacrolimus with a target trough level (e.g., target trough blood level) of approximately 1 ng/mL or more, approximately 2 ng/mL or more, approximately 3 ng/mL or more, approximately 4 ng/mL or more, approximately 5 ng/mL or more, approximately 6 ng/mL or more, approximately 7 ng/mL or more, approximately 8 ng/mL or more, approximately 9 ng/mL or more, approximately 10 ng/mL or more, approximately 11 ng/mL or more, approximately 12 ng/mL or more, approximately 13 ng/mL or more, approximately 14 ng/mL or more, approximately 15 ng/mL or more, approximately 16 ng/mL or more, approximately 17 ng/mL or more, approximately 18 ng/mL or more, approximately 19 ng/mL or more, or approximately 20 ng/mL or more.

In some embodiments, a GVHD prophylactic agent of the present disclosure is tacrolimus, and the tacrolimus is provided at a dose that ranges from approximately 0.01 mg per kilogram of body weight of a subject receiving the agent to approximately 0.50 mg per kilogram of body weight of a subject receiving the agent twice per day. In some embodiments, the tacrolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.

A single GVHD prophylactic agent can be sirolimus. In some embodiments, a low dose of a GVHD prophylactic agent is sirolimus with a target trough level (e.g., trough blood level) of approximately 3 ng/mL to approximately 8 ng/mL. In some embodiments, a low dose of a GVHD prophylactic agent is sirolimus with a target trough level of approximately 4 ng/mL to approximately 8 ng/mL. In some embodiments, a low dose of a GVHD prophylactic agent is sirolimus with a target trough level (e.g., target trough blood level) of approximately 1 ng/mL or more, approximately 2 ng/mL or more, approximately 3 ng/mL or more, approximately 4 ng/mL or more, approximately 5 ng/mL or more, approximately 6 ng/mL or more, approximately 7 ng/mL or more, approximately 8 ng/mL or more, approximately 9 ng/mL or more, approximately 10 ng/mL or more, approximately 11 ng/mL or more, approximately 12 ng/mL or more, approximately 13 ng/mL or more, approximately 14 ng/mL or more, approximately 15 ng/mL or more, approximately 16 ng/mL or more, approximately 17 ng/mL or more, approximately 18 ng/mL or more, approximately 19 ng/mL or more, or approximately 20 ng/mL or more.

In some embodiments, a GVHD prophylactic agent of the present disclosure is sirolimus, and the sirolimus is provided at a dose that ranges from approximately 0.01 mg per kilogram of body weight of a subject receiving the agent to approximately 0.50 mg per kilogram of body weight of a subject receiving the agent twice per day. In some embodiments, the sirolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.

Methods for alloHCT of the disclosure can utilize tacrolimus. Combining tacrolimus with one or more cell populations in an alloHCT regimen as disclosed herein can result in surprising improvements in clinical outcomes. Methods for alloHCT of the disclosure without utilizing tacrolimus, as GVHD prophylaxis, can result in surprising improvements in clinical outcomes.

An aspect of the present disclosure provides a pharmaceutical treatment or cellular therapy product to be administered to a human subject in need thereof. In some embodiments, the pharmaceutical treatment comprises CD34+ cells (e.g., HSPC), Treg, iNKT, and Tmem, and wherein the population of cells is depleted of naïve conventional αβ-T cells. The pharmaceutical treatment can further comprise a solution comprising one or more doses of a graft vs host disease (GVHD) prophylactic agent, such as tacrolimus. In some embodiments, the cellular therapy product comprising a population of isolated CD45⁺ cells, wherein the population of isolated CD45⁺ cells comprises a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the cellular therapy product has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. The cellular therapy product can further comprise a solution comprising one or more doses of a graft vs host disease (GVHD) prophylactic agent, such as tacrolimus.

In some embodiments, the tacrolimus is administered or dosed in an amount to maintain or that maintains a target blood level of at least about 3 ng/ml for at least about 20 days after administering the population of cells comprising HSPC, in an amount to maintain a target blood level of about 4 ng/ml or more for at least about 40 days after administering the population of cells comprising HSPC, and/or in an amount that maintains a target blood level of about 4 ng/ml or more for at least about 40 days after administering the population of cells comprising HSPC. In some cases, the tacrolimus is administered in an amount that maintains a target blood level of at most about 10 ng/ml for at least 30 days after administering the population of cells comprising HSPC. In some embodiments, the tacrolimus is administered for at least about 60 days after administering the population of cells comprising HSPC, for at least about 90 days after administering the population of cells comprising HSPC, for at most about 150 days after administering the population of cells comprising HSPC, for at most about 120 days after administering the population of cells comprising HSPC.

In some embodiments, the tacrolimus is administered or dosed in amount to maintain or that maintains a target blood level of approximately 1 ng/mL to approximately 10 ng/mL in the human subject receiving the product for approximately 5 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 105 days or more, approximately 110 days or more, approximately 115 days or more, approximately 120 days or more, approximately 125 days or more, approximately 130 days or more, approximately 135 days or more, approximately 140 days or more, approximately 145 days or more, or approximately 150 days or more after administration of the population of isolated CD45⁺ cells.

In some embodiments, the tacrolimus is administered for at least about 60 days after administering the population of isolated CD45⁺ cells, for at least about 90 days after administering the population of isolated CD45⁺ cells, for at most about 150 days after administering the population of isolated CD45⁺ cells, or for at most about 120 days after administering the population of isolated CD45⁺ cells.

In some embodiments, the tacrolimus is formulated for oral administration or for intravenous administration.

In embodiments, a herein-disclosed method further comprises administering to the patient over a period of time up to approximately 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced. In various embodiments, the threshold level is above approximately 4 ng of tacrolimus per ml of patient blood or the threshold level is above approximately 5 ng of tacrolimus per ml of patient blood. In some embodiments, the tacrolimus GHVDPA is administered to maintain a concentration of tacrolimus in the patients' blood below an upper threshold level during the period of time. In some cases, the upper threshold level is below approximately 10 ng of tacrolimus per ml of patient blood.

Tacrolimus is a macrolide that can exhibit immunosuppressive activity in vivo, and can prevent or reduce the activation of T-lymphocytes in response to antigenic or mitogenic stimulation. Treatment with tacrolimus as a single-agent prophylactic can result in, for example, decreased cytokine production and decreased T cell signal transduction. Tacrolimus can bind to the FKBP-12 protein and form a complex with calcium-dependent proteins, thereby inhibiting calcineurin phosphatase activity. This prevents or reduces the dephosphorylation and translocation of nuclear factor of activated T-cells (NFAT), a nuclear component thought to initiate gene transcription for the expression of lymphokines. Tacrolimus also inhibits the transcription for genes which encode IL-3, IL-4, IL-5, GM-CSF, and TNF, all of which are involved in the early stages of T-cell activation. Tacrolimus can therefore be used to reduce the risk of GVHD in alloHCT recipient subjects. However, methods disclosed herein—that utilize tacrolimus as a single-agent prophylactic—achieve superior clinical outcomes to those observed in other alloHCT methods that utilize tacrolimus GVHD prophylactic. In some embodiments, methods disclosed herein-do not utilize tacrolimus as a single-agent prophylactic-achieve superior clinical outcomes to those observed in other alloHCT methods that utilize tacrolimus GVHD prophylactic. For example, in some embodiments alloHCT methods of the disclosure that utilize tacrolimus achieve superior relapse-free survival compared to a standard of care regimen that comprises more potent GVHD prophylaxis with methotrexate plus tacrolimus, and even compared to an alloHCT method that utilizes a drug with a similar target and mechanism of action (sirolimus). Thus, although tacrolimus can be referred to as a GVHD prophylactic herein, it can also contribute to additional therapeutic effects beyond or not directly related to GVHD prophylaxis. Therefore, as used herein, the term “tacrolimus as a single-agent GVHD prophylactic” also “includes tacrolimus as a single-agent prophylactic for additional therapeutics effects.” In other words, the term “tacrolimus as a single-agent prophylactic” and “tacrolimus as a single-agent GVHD prophylactic” are synonyms.

In some embodiments, tacrolimus as a single-agent prophylactic is administered to a subject orally. Absorption of tacrolimus from the gastrointestinal tract after oral administration can be incomplete and variable. The absolute bioavailability of tacrolimus in healthy subjects after oral administration can be 18±5%. The rate and extent of absorption can vary based on whether tacrolimus is given with food. In some embodiments, tacrolimus is administered parenterally, for example, intravenously or subcutaneously. In some embodiments, tacrolimus is administered by a topical, intramuscular, intradermal, intraperitoneal, intraspinal, or epidural route. Tacrolimus can be administered as an extended release formulation. In some embodiments, tacrolimus is used as a free base. In some embodiments, tacrolimus is used as a pharmaceutically-acceptable salt.

In some embodiments, methods of the disclosure can allow low doses of tacrolimus to be used. In some embodiments, a low dose of tacrolimus can improve donor T cell chimerism in a subject. In some embodiments, a low dose of tacrolimus can improve alloHCT engraftment as disclosed herein. In some embodiments, a low dose of tacrolimus can reduce the incidence or relative risk of adverse effects that can be associated with high doses of tacrolimus, such as blurred vision, liver and kidney toxicity, seizures, tremors, hypertension, hypomagnesemia, diabetes mellitus, hyperkalemia, itching, insomnia, and confusion.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the population of isolated CD45⁺ cells, as disclosed herein. In some embodiments, the tacrolimus is initially administered to the human subject at a dose that ranges from approximately 0.01 mg/kg human subject's actual or ideal body weight/day to approximately 0.50 mg/kg human subject's actual or ideal body weight/day or the tacrolimus is initially administered from approximately 12 hours to approximately 24 hours after the administering of the population of isolated CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering of the population of isolated CD45⁺ cells, as disclosed herein.

A circulating level of tacrolimus can be monitored, and doses adjusted accordingly to achieve a target concentration. For example, a whole blood concentration of tacrolimus can be monitored, and doses adjusted and administered to achieve a target trough level. A target trough level of tacrolimus can be, for example, less than about 25 ng/mL, less than about 24 ng/mL, less than about 23 ng/mL, less than about 22 ng/mL, less than about 21 ng/mL, less than about 20 ng/mL, less than about 19 ng/mL, less than about 18 ng/mL, less than about 17 ng/mL, less than about 16 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a target trough level (e.g., trough blood level) of tacrolimus is about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a target trough level of (e.g., trough blood level) tacrolimus is about 6 ng/mL to about 10 ng/mL. In some embodiments, a target trough level of tacrolimus is about 6 ng/mL to about 9 ng/mL. In some embodiments, a target trough level of tacrolimus is about 6 ng/mL to about 8 ng/mL. In some embodiments, a target trough level of tacrolimus is about 5 ng/mL to about 10 ng/mL. In some embodiments, a target trough level of tacrolimus is about 5 ng/mL to about 9 ng/mL. In some embodiments, a target trough level of tacrolimus is about 5 ng/mL to about 8 ng/mL. In some embodiments, a target trough level of tacrolimus is about 4 ng/mL to about 10 ng/mL. In some embodiments, a target trough level of tacrolimus is about 4 ng/mL to about 9 ng/mL. In some embodiments, a target trough level of tacrolimus is about 4 ng/mL to about 8 ng/mL.

In some embodiments, a dose of tacrolimus as a single-agent prophylactic or a target trough level (e.g., trough blood level) of tacrolimus can be adjusted based on a clinical parameter disclosed herein. For example, in some cases, a dose or a target trough level of tacrolimus can be reduced if a subject exhibits lower donor T cell chimerism than desired, e.g., a percent of peripheral blood donor-derived CD3+ cells that is less than 95%, less than 90%, less than 85%, less than 80%, less than 75%, less than 70%, less than 65%, less than 60%, less than 55%, less than 50%, or less than 45% when evaluated after a suitable amount of time after administration of a cell population of the disclosure, for example, at about 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, 180 days, or 1 year after administration of a cell population of the disclosure. In some embodiments, a target trough level of tacrolimus can be increased if a subject exhibits signs of GVHD as disclosed herein.

In some embodiments, a subject achieves at least about 80% chimerism at about day 30. In some embodiments, a subject achieves at least about 80% chimerism at about day 30 and has a target trough level of tacrolimus of between about 6.5 ng/mL and about 9 ng/mL.

Administration of tacrolimus to a subject can commence before or after administration of a cell population disclosed herein (e.g., a population of isolated CD45⁺ cells). In some embodiments, administration of tacrolimus to a subject commences before administration of a cell population disclosed herein. In some embodiments, administration of tacrolimus to a subject commences after administration of a cell population disclosed herein. In some embodiments, administration of tacrolimus to a subject commences at about the same time as administration of a cell population disclosed herein.

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days before administration of a cell population disclosed herein (e.g., a population of isolated CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days before administration of a cell population disclosed herein (e.g., a population of isolated CD45⁺ cells). In some embodiments, administration of tacrolimus to a subject commences 1 day, 2 days, 3 days, 4 days, 5 days, 6 days, 7 days, 10 days, 14 days, or 20 days before administration of a cell population disclosed herein (e.g., a population of isolated CD45⁺ cells). In some embodiments, administration of tacrolimus to a subject commences 1 day before administration of a population of isolated CD45⁺ cells.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering a population of cells of the present disclosure (e.g., a population of isolated CD45⁺ cells).

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days after administration of a cell population disclosed herein (e.g., a population of isolated CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days after administration of a cell population disclosed herein (e.g., a population of isolated CD45⁺ cells). In some embodiments, administration of tacrolimus to a subject commences 1 day, 2 days, 3 days, 4 days, 5 days, 6 days, 7 days, 10 days, 14 days, or 20 days after administration of a cell population disclosed herein (e.g., a population of isolated CD45⁺ cells). In some embodiments, administration of tacrolimus to a subject commences 1 day after administration of a population of isolated CD45⁺ cells s. In some embodiments, administration of tacrolimus to a subject commences the same day as a cell population of the disclosure is administered.

Tacrolimus can be administered to a subject for any amount of time after administration of a cell population of the disclosure (e.g., a population of isolated CD45⁺ cells). In some embodiments, tacrolimus is administered to a subject for the first 7 days, 14 days, first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years after administration of a cell population of the disclosure (e.g., a population of isolated CD45⁺ cells).

In some embodiments, tacrolimus is administered to a subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years after administration of a cell population of the disclosure (e.g., a population of isolated CD45⁺ cells).

In some embodiments, a dose of the tacrolimus is tapered starting at about 90 days after the first dose is administered to the human subject. In some embodiments, a dose of the tacrolimus is tapered starting at about 70 days after the first dose is administered to the human subject. In some embodiments, a dose of the tacrolimus is tapered starting at about 60 days after the first dose is administered to the human subject. In some embodiments, a dose of the tacrolimus is tapered starting at about 50 days after the first dose is administered to the human subject, or a dose of the tacrolimus is tapered starting at about 45 days after the first dose is administered to the human subject. In some embodiments, tacrolimus is tapered at approximately 20% of the dose per month. In some embodiments, completion of a corticosteroid taper should be considered before tapering of tacrolimus.

In some embodiments, a subject achieves at least about 80% chimerism at about day 30. In some embodiments, a subject achieves at least about 80% chimerism at about day 30 and has a target trough level is of about 6.5 ng/mL and about 9 ng/mL.

### Subjects

Provided herein are compositions (e.g., cellular therapeutic products) for administration to a recipient subject having a cancer or autoimmune disorder, and methods of administering the same. The compositions and methods can be useful for treating or reducing cancer or treating an autoimmune disorder in the subject.

In some embodiments, a subject is at least 3, at least 4, at least 5, at least 6, at least 7 at least 8 at least 9 at least 10, at least 11, or at least 12 months of age. In some embodiments, a subject is at least 1, least 2, least 3, least 4, least 5, least 6, at least 7, at least 8, at least 9, at least 10, at least 11, at least 12, least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 years of age. In some embodiments, a subject is approximately 18 years of age or older. In some embodiments, a subject is approximately 16 years of age or older. In some embodiments, a subject is approximately 13 years of age or older. In some embodiments, a subject is approximately 6 years of age or older. In some embodiments, a subject is approximately 3 years of age or older. In some embodiments, a subject is approximately 1 year of age or older. In some embodiments, a subject is approximately 9 months of age or older. In some embodiments, a subject is approximately 6 months of age or older. In some embodiments, a subject is approximately 3 months of age or older.

In some embodiments, a subject is at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, or at most 80 years of age. In some embodiments, a subject is at most 65 years of age. In some embodiments, a subject is at most 70 years of age. In some embodiments, a subject is at most 75 years of age.

In some embodiments, a subject is from between approximately 3 months to approximately 18 years of age. In some embodiments, a subject is from approximately 18 years to approximately 65 years of age. In some embodiments, a subject is from approximately 18 years to approximately 75 years of age. In some embodiments, a subject is from approximately 66 years to approximately 75 years of age.

In some embodiments, the subject has received from one to five previous lines of therapy prior to administration of a composition, treatment, and/or therapy of the present disclosure. In some embodiments, the subject has received five previous lines of therapy prior to administration of a composition, treatment, and/or therapy of the present disclosure. In some embodiments, the subject has received four previous lines of therapy prior to administration of a composition, treatment, and/or therapy of the present disclosure. In some embodiments, the subject has received three previous lines of therapy prior to administration of a composition, treatment, and/or therapy of the present disclosure. In some embodiments, the subject has received two previous lines of therapy prior to administration of a composition, treatment, and/or therapy of the present disclosure. In some embodiments, the subject has received one previous line of therapy prior to administration of a composition, treatment, and/or therapy of the present disclosure. In some embodiments, the subject is or has become refractory to one or more of the previous lines of therapy prior to administration of a composition, treatment, and/or therapy of the present disclosure. In some embodiments, the subject is or has become refractory to two or more of the previous lines of therapy prior to administration of a composition, treatment, and/or therapy of the present disclosure. In some embodiments, the subject is or has become refractory to three or more of the previous lines of therapy prior to administration of a composition, treatment, and/or therapy of the present disclosure. In some embodiments, the subject is or has become refractory to four or more of the previous lines of therapy prior to administration of a composition, treatment, and/or therapy of the present disclosure. In some embodiments, the subject is or has become refractory to all previous lines of therapy prior to administration of a composition, treatment, and/or therapy of the present disclosure.

### Conditions

Another aspect provides a method of treating a human subject having (e.g., diagnosed with) or suspected of having a hematologic malignancy. In one embodiment, the method comprises administering to the human subject a composition or treatment of the present disclosure (e.g., a cellular therapy product or cell population of the present disclosure). In one embodiment, the method comprises administering to the human subject a solution comprising a cell population that comprise CD34+ cells (e.g., HSPC), Treg, and Tmem, and is depleted of naïve conventional αβ-T cells or naïve conventional CD3⁺CD25⁻CD45RA⁺ T cells (Tcon), and optionally, a solution comprising one or more doses of at least one GVHD prophylactic agent of the present disclosure (e.g., tacrolimus). In one embodiment, the method comprises administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon).

In some embodiments, a hematologic malignancy that may be treated by a method of the present disclosure includes, without limitation, leukemia, acute leukemia, acute leukemia in complete remission, active acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, high-risk or very high-risk myelodysplastic syndrome, myeloproliferative syndrome, myelofibrosis, blastic plasmacytoid dendritic cell neoplasm (BPDCN), and any combinations thereof. In some embodiments, the human subject has been diagnosed with a histopathologically confirmed hematologic malignancy of the present disclosure. In some embodiments, the hematologic malignancy risk category (e.g., low risk, intermediate risk, high risk, very high risk) may be determined as per the Center for International Blood & Marrow Transplant Research (CIBMTR) Disease Risk Index (DRI).

In some embodiments, a myelodysplastic syndrome (MDS) of the present disclosure is one that is indicated for allogeneic hematopoietic cell transplant (alloHCT) per the 2017 International Expert Panel recommendations (de Witte 2017) and/or is a therapy-related/secondary MDS as defined by the World Health Organization classification of myeloid malignancies (WHO 2017).

In some embodiments, the acute leukemia is in complete remission with incomplete hematologic recovery (CRi). In any of the preceding embodiments, minimal residual disease may or may not be present in the human subject. In some embodiments, the human subject has approximately 20% or less, approximately 19% or less, approximately 18% or less, approximately 17% or less, approximately 16% or less, approximately 15% or less, approximately 14% or less, approximately 13% or less, approximately 12% or less, approximately 11% or less, approximately 10% or less, approximately 9% or less, approximately 8% or less, approximately 7% or less, approximately 6% or less, or approximately 5% or less blast burden in their bone marrow. In some embodiments, there is an absence of circulating blasts and/or blasts with Auer rods in the human subject. In some embodiments, there is an absence of extramedullary disease in the human subject. In some embodiments, the human subject has an absolute neutrophil count (ANC) that is approximately 1.0×10⁹/L or higher. In some embodiments, the human subject has a platelet count that is approximately 1.0×10¹¹/L or higher. In some embodiments, the human subject is red blood cell-transfusion-independent. In some embodiments, the human subject has residual neutropenia. In some embodiments, the human subject has an absolute neutrophil count (ANC) that is approximately 1.0×10⁹/L or lower. In some embodiments, the human subject has residual thrombocytopenia. In some embodiments, the human subject has a platelet count that is approximately 1.0×10¹¹/L or lower. In some embodiments, the acute leukemia is categorized as intermediate-risk to high-risk acute leukemia or very high-risk leukemia. In some embodiments, the acute leukemia is active acute leukemia. In some embodiments, the human subject has approximately 10% or less leukemic blast infiltration of bone marrow. The acute leukemia may be acute myeloid leukemia (AML), acute lymphoid leukemia (ALL), and/or mixed phenotype acute leukemia (MPAL). In some embodiments, the ALL is B cell ALL. In some embodiments, the high risk AML comprises a complex karyotype with 3 or more clonal chromosomal abnormalities. For example, 3 or more clonal chromosomal abnormalities may include monosomal karyotype-5, 5q-, -7 or 7q-, t(11q23, t(9;11), inv(3), t(3,3)t(6;9)t(9;22), normal karyotype with a fins-like tyrosine kinase 3 (FLT3)-ITD mutation, and any combination thereof. In some embodiments, the CML is in blast phase, is in second chronic phase, is in chronic phase, is accelerated, has a history of blast crisis, and/or is resistant to intolerant of one or more first-generation or second-generation tyrosine kinase inhibitors (TKIs). In some embodiments, the therapy-related myelodysplastic syndrome and/or secondary myelodysplastic syndrome is in complete remission and is categorized as intermediate-risk to high-risk.

In some embodiments, the human subject has or is suspected of having chronic myelogenous leukemia (CML). In some embodiments, In some embodiments, the human subject is in myeloid blast crises and/or lymphoid blast crisis. In some embodiments, the blast crisis is in complete remission. In some embodiments, the blast crisis is in complete remission with incomplete hematologic recovery. In some embodiments, the CML is in accelerated phase. In some embodiments, the CML is in chronic phase. In some embodiments, the chronic phase CML is resistant to or intolerant of one or more first-generation or second-generation tyrosine kinase inhibitors (TKIs).

In some embodiments, the human subject has or is suspected of having high-risk or very high-risk myelodysplastic syndrome. In some embodiments, the human subject has approximately 10% or less blast burden in their bone marrow.

In some embodiments, the human subject has or is suspected of having myelofibrosis. In some embodiments, the myelofibrosis is eligible for standard myeloablative allogeneic hematopoietic stem cell transplant (alloHSCT). In some embodiments, the myelofibrosis is intermediate-2-risk myelofibrosis or high-risk myelofibrosis. In some embodiments, the myelofibrosis is intermediate-1-risk myelofibrosis associated with high symptom burden, low platelet counts, and/or complex cytogenetics.

Another aspect provides a method of treating a human subject having (e.g., diagnosed with) or suspected of having an autoimmune disorder. In one embodiment, the method comprises administering to the human subject a composition or treatment of the present disclosure (e.g., a cellular therapy product or cell population of the present disclosure). In one embodiment, the method comprises administering to the human subject a solution comprising a cell population of the present disclosure that comprise CD34+ cells (e.g., HSPC), Treg, and Tmem, and is depleted of naïve conventional αβ-T cells, and optionally, a solution comprising one or more doses of at least one GVHD prophylactic agent of the present disclosure (e.g., tacrolimus). In one embodiment, the method comprises administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon). In one embodiment, the autoimmune disorder is multiple sclerosis. In some embodiments, the multiple sclerosis is primary progressive multiple sclerosis (PPMS). In some embodiments, the multiple sclerosis is chronic progressive multiple sclerosis. In some embodiments, the multiple sclerosis is secondary progressive multiple sclerosis. In some embodiments, the multiple sclerosis is relapsing-remitting multiple sclerosis. In some embodiments, the multiple sclerosis is acute relapsing multiple sclerosis.

In some embodiments, a cellular therapy product of the present disclosure increases GVHD-free survival. In some embodiments, a cellular therapy product of the present disclosure increases event-free survival (EFS). In some embodiments, a human subject administered the cell therapy product does not experience loss of neurological function, does not experience relapse, does not develop 2 or more new and/or larger lesions, and/or does not experience death. In some embodiments, a cellular therapy product of the present disclosure increases overall survival. In some embodiments, a cellular therapy product of the present disclosure decreases treatment related mortality (TRM). In some embodiments, a cellular therapy product of the present disclosure decreases or stabilizes disease activity. In some embodiments, a cellular therapy product of the present disclosure decreases incidence and/or severity of acute GVHD. In some embodiments, a cellular therapy product of the present disclosure decreases wherein incidence, severity, timing, or any combination thereof of Grade II to Grade IV acute GVHD. In some embodiments, a cellular therapy product of the present disclosure decreases incidence and/or severity of chronic GVHD. In some embodiments, a cellular therapy product of the present disclosure decreases incidence, severity, timing, or any combination thereof of moderate to severe chronic GVHD. In some embodiments, a cellular therapy product of the present disclosure decreases incidence of secondary graft failure. In some embodiments, a cellular therapy product of the present disclosure decreases incidence of Grade 3 or higher infectious disease complications. In some embodiments, a cellular therapy product of the present disclosure modulates or otherwise decreases one or more multiple sclerosis biomarkers (e.g., oligoclonal bands protein level and/or neurofilament protein level in cerebrospinal fluid (CSF)). In some embodiments a cellular therapy product of the present disclosure improves quality of life. In some embodiments a cellular therapy product of the present disclosure improves one or more radiographic markers of PPMS.

In some embodiments, for acute myeloid leukemia and mixed phenotype acute leukemia, a complete response/complete remission (CR) may be according to FDA draft guidance for industry (“Acute Myeloid Leukemia: Developing Drugs and Biological Products for Treatment (August 2020)”. In some embodiments, a CRi may comprise meeting all CR criteria except for residual neutropenia or thrombocytopenia. In some embodiments, for acute lymphoblastic leukemia (ALL), a CR is defined per the Center for International Blood and Marrow Transplant Research (CIBMTR 2021). In some embodiments, a CRi may comprise meeting all CR criteria except for residual neutropenia or thrombocytopenia.

A further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPC) to be administered to the patient; the population of HSPC comprising HSPC and a liquid suspending the HSPC; providing a population of regulatory T cells (Treg) to be administered to the patient, the population of Treg comprising Treg and a liquid suspending the Treg; providing a population of memory T cells (Tmem) to be administered to the patient, the population of Tmem comprising Tmem and a liquid suspending the Tmem; and wherein the population of cells is depleted of naïve conventional αβ-T cells or naïve conventional CD3⁺CD25⁻CD45RA⁺ T cells (Tcon), and after administration of the cell populations, the patient has a reduced risk and/or severity of the adverse event as compared to hematologic malignancy patients who did not receive the cell populations provided herein.

Another aspect provides a method of transplanting cell populations into a human patient as a part of a treatment regimen for a hematologic malignancy. The method comprises administering to the patient a population of hematopoietic stem and progenitor cells (HSPC; the population of HSPC comprising HSPC and a liquid suspending the HSPC; administering to the patient a population of regulatory T cells (Treg) to be administered to the patient, the population of Treg comprising Treg and a liquid suspending the Treg; administering to the patient a population of memory T cells (Tmem) to be administered to the patient, the population of Tmem comprising Tmem and a liquid suspending the Tmem; and administering to the patient a population of cells that is depleted of naïve conventional αβ-T cells or naïve conventional CD3⁺CD25⁻CD45RA⁺ T cells (Tcon) and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

The methods of the disclosure can be used for treating a subject (e.g., a human subject) with a cancer. In some embodiments, the subject has been treated for cancer, e.g., by treatment with a chemotherapeutic drug or with radiation. The methods of the disclosure can be useful for treating a hematologic malignancy, for example, leukemia or lymphoma. Examples of hematologic malignancies that can be treated by the methods of the disclosure include, but are not limited to, acute lymphocytic leukemia (ALL), acute myelogenous leukemia (AML), chronic myelogenous leukemia (CML), multiple myeloma, and lymphomas such as Hodgkin and non-Hodgkin lymphomas. A cancer can be a solid tumor. In some embodiments, the cancer is a primary or metastatic tumor.

The types of cancer that can be treated using the methods of the present disclosure include but are not limited to leukemia, lymphoma, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, nonmelanoma skin cancers, stomach cancer, testicular cancer; thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, and Waldenstrom's macroglobulinemia.

Patients with high-risk hematologic malignancies are rarely cured with standard chemotherapy. High-risk malignancies include, for example, leukemia or lymphoma that has progressed beyond first remission, or leukemia or lymphoma with refractory relapse.

In some embodiments, the human subject or patient has previously been or is concurrently treated for the hematologic malignancy.

A subject that receives a composition of the disclosure can have, for example, acute myeloid leukemia, acute lymphoid leukemia, mixed phenotype leukemia, myelofibrosis, high-risk myelodysplastic syndrome, very high-risk myelodysplastic syndrome, myelofibrosis (MF) that is eligible for transplant per National Comprehensive Cancer Network Guidelines, intermediate-2- or high-risk MF according to the IPSS, DIPSS or DIPSS-plus scoring systems, intermediate-1-risk MF associated with high-risk features such as high symptoms burden, low platelet counts, or complex cytogenetics, primary myelofibrosis, myelofibrosis evolved from another myeloproliferative neoplasm, myelodysplastic syndrome, non-Hodgkin lymphoma, a non-malignant indication for allogeneic hematopoietic stem cell transplantation (alloHCT),

In some embodiments, a subject has acute myeloid leukemia. In some embodiments, a subject has acute lymphoid leukemia. In some embodiments, a subject has mixed phenotype leukemia. In some embodiments, a subject has high-risk myelodysplastic syndrome. In some embodiments, a subject has very high-risk myelodysplastic syndrome. In some embodiments, a subject has myelofibrosis (MF) that is eligible for transplant per National Comprehensive Cancer Network Guidelines. In some embodiments, a subject has intermediate-2- or high-risk myelofibrosis according to the IPSS, DIPSS or DIPSS-plus scoring systems. In some embodiments, a subject has intermediate-1-risk myelofibrosis associated with high-risk features such as high symptoms burden, low platelet counts, or complex cytogenetics. In some embodiments, a subject has primary myelofibrosis. In some embodiments, a subject has myelofibrosis. In some embodiments, a subject has myelofibrosis evolved from another myeloproliferative neoplasm. In some embodiments, a subject has myelodysplastic syndrome. In some embodiments, a subject has non-Hodgkin lymphoma. In some embodiments, a subject has a non-malignant indication for alloHCT.

A subject can be in complete remission (CR). A subject can be in complete remission with incomplete hematologic recovery (CRi), e.g., without the presence of known minimal residual disease. A subject can have minimal residual disease. A subject can have no evidence of minimal residual disease. A subject can have active disease. A subject can have a leukemia (e.g., acute myeloid, acute lymphoid, or mixed phenotype) that is not in morphologic CR with bone marrow infiltration by leukemic blasts of 510%. A subject can have a leukemia (e.g., acute myeloid, acute lymphoid, or mixed phenotype) that is in morphologic CR with evidence of minimal residual positivity by either multiparameter flow cytometric analysis or by a nucleic acid-based technique.

Complete remission (CR) for acute myeloid, lymphoid or mixed phenotype leukemia can be indicated by meeting all of the following criteria: (i) Bone marrow blasts <5%; (ii) Absence of circulating blasts and blasts with Auer rods; (ii) Absence of extramedullary disease 4. ANC≥1.0×10⁹/L (1,000/μL); (iii) Platelet count ≥100×10⁹/L (100,000/μL); and (iv) Independence of red cell transfusions. Complete Response with Incomplete Hematologic Recovery (CRi) can be indicated by meeting all the CR criteria except for residual neutropenia (<1.0×10⁹/L) or thrombocytopenia (<100×10⁹/L).

In some embodiments, the methods of the present disclosure may further include administering one or more additional therapeutic agents. Examples, of such therapeutic agents include, without limitation, an inhibitor of an immunosuppressive enzymes, e.g., narginase II and indoleamine 2,3-dioxygenase 1 (IDO1); a promflammatory cytokine, e.g., IL-2 or IL-15; a blocker of an anti-inflammatory cytokine, e.g., an antibody that binds to and/or blocks M-CSF, IL-4, or TGF-beta; a bispecific antibody, e.g., that targets a protein or proteins relating to Natural Killer (NK) function, such as an NKG2D-based or CD16-based bispecific antibody; a check-point inhibitor, e.g., a CTLA4 inhibitor, a PD 1 inhibitor, a PD-L 1 inhibitor, a CD47/SIRP-alpha inhibitor, a LAG-3 inhibitor, a TIM-3 inhibitor, a B7-H3 inhibitor, a B7-H4 inhibitor, an A2aR inhibitor, a CD73 inhibitor, an NKG2A inhibitor, a PVRIG/PVRL2 inhibitor, a CEACAM1 inhibitor, a CEACAM 5/6 inhibitor, a FAK inhibitor, a CCL2/CCR2 inhibitor, a LIF inhibitor, a CSF-1 inhibitor, an IL-8 inhibitor, a SEMA4D inhibitor, an Ang-2 inhibitor, a CLEVER-1 inhibitor, an Axl inhibitor, a phsphatidylserine inhibitor, and/or a TIGIT inhibitor; a JAK/STAT inhibitor, e.g., a Jak2 inhibitor; a FLT3 inhibitor; and one or more CD45⁺ cells, e.g., one or more T cells, NK cells, CTLs, Tregs, or NKT cells, comprising one or more chimeric receptors. In some embodiments, the chimeric receptor comprises one or more extracellular antigen-binding domains that bind to one or more cancer-associated antigens. Any suitable cancer-associated antigen known in the art may be targeted. In some embodiments, the chimeric receptor is a chimeric antigen receptor or a T cell receptor.

### Sensitivities

In some embodiments, a subject does not have a known allergy or hypersensitivity to, or intolerance of, tacrolimus. In some embodiments, a subject does not have a known allergy or hypersensitivity to, or intolerance of, sirolimus.

In some embodiments, subjects are not sensitive to iron dextran (e.g., subjects with sensitivity to iron dextran are not eligible to receive a composition of the disclosure. In some cases, this may be because of the magnetic beads used in some embodiments to isolate, deplete, and/or purify cell types). In some embodiments, subjects are not sensitive to products derived from cyanine dyes (e.g., subjects with sensitivity to products derived from cyanine dyes are not eligible to receive a composition of the disclosure).

In some embodiments, subjects are not sensitive to proteins products derived from murine sources (e.g., subjects with sensitivity to proteins products derived from murine sources are not eligible to receive a composition of the disclosure).

In some embodiments, subjects are not sensitive to proteins products derived from bovine sources (e.g., subjects with sensitivity to proteins products derived from bovine sources are not eligible to receive a composition of the disclosure).

In some embodiments, subjects are not sensitive to proteins products derived from algal sources (e.g., subjects with sensitivity to proteins products derived from algal sources are not eligible to receive a composition of the disclosure).

In some embodiments, subjects are not sensitive to proteins products derived from Streptomyces avidinii (e.g., subjects with sensitivity to proteins products derived from Streptomyces avidinii are not eligible to receive a composition of the disclosure).

### Organ Function and Biomarkers

A subject can have an estimated glomerular filtration rate (eGFR)>30 mL/minute. A subject can have an estimated glomerular filtration rate (eGFR)>40 mL/minute. A subject can have an eGCF of >50 mL/minute. A subject can have an estimated glomerular filtration rate (eGFR)>60 mL/minute.

A subject can have a cardiac ejection fraction at rest ≥45%, or shortening fraction of ≥27% by echocardiogram or radionuclide scan (MUGA).

A subject can have a diffusing capacity of the lung for carbon monoxide (DLCO) (adjusted for hemoglobin) of ≥50%.

A subject can have a negative serum or urine beta-HCG test, e.g., in females of childbearing potential within 3 weeks of registration.

A subject can have total bilirubin <2 times upper limit of normal (ULN).

A subject can have Gilbert's syndrome, wherein hemolysis has been excluded.

A subject can have an ALT reading within 3 times upper limit of normal (ULN). A subject can have an AST reading within 3 times upper limit of normal (ULN).

### Additional Therapies and Other Subject Characteristics

In some embodiments, a subject has not received a prior alloHCT. In some embodiments, a subject is not a candidate for autologous transplant. In some embodiments, a subject is not receiving corticosteroids or other immunosuppressive therapy. In some embodiments, a subject is receiving topical corticosteroids or oral systemic corticosteroid doses less than or equal to 10 mg/day. In some embodiments, a subject does not receive donor lymphocyte infusion (DLI). In some embodiments, a subject does not receive a T cell depleting pharmaceutical, e.g., post-transplant cyclophosphamide (Cy), peri-transplant anti-thymocyte globulin (ATG), or alemtuzumab. In some embodiments, a subject that has previously been exposed to a T cell-depleting agent has a 5 half-life washout of the agent prior to planned transplant day 0 (day of infusion of the Treg and HSPC components of the graft).

In some embodiments, a subject is not positive for anti-donor HLA antibodies against a mismatched allele in the selected donor as determined by either: (a) A positive crossmatch test of any titer; or (b) The presence of anti-donor HLA antibody to any HLA locus. In some embodiments, the subject has a Karnofsky performance score ≥70%. In some embodiments, a subject does not have a hematopoietic cell transplantation-specific Comorbidity Index (HCT-CI) of >4. In some embodiments, a subject does not have an uncontrolled bacterial, viral, or fungal infection. In some embodiments, a subject is not taking antimicrobial therapy and with progression or no clinical improvement in infection. In some embodiments, a subject is not seropositive for HIV-1 or -2, HTLV-1 or -2, Hepatitis B sAg, or Hepatitis C antibody. In some embodiments, a subject does not have an uncontrolled autoimmune disease that requires active immunosuppressive treatment. In some embodiments, a subject does has not had concurrent malignancies or active disease within 1 year, for example, excluding non-melanoma skin cancers that have been curatively resected. In some embodiments, a subject does not exhibit psychosocial circumstances that preclude the patient being able to go through transplant or participate responsibly in follow up care. In some embodiments, a subject is not pregnant or breastfeeding. In some embodiments, a subject does not have a serious medical condition or abnormality in clinical laboratory tests that, in the medical professional's judgment, precludes the subject's safety upon receipt of a composition of the disclosure. In some embodiments, a subject is eligible for myeloablative alloHCT.

In some embodiments, a subject receives a prophylactic agent to reduce the risk of bacterial, fungal, and/or viral infection, e.g., during the pen-transplant period.

In some embodiments, a subject receives a supportive therapy for HCT-related toxicity. In some embodiments, a subject does not receive a supportive therapy for HCT-related toxicity. In some embodiments, a subject receives a growth factor. In some embodiments, a subject does not receive a growth factor. In some embodiments, a subject receives intravenous immunoglobulin. In some embodiments, a subject does not receive intravenous immunoglobulin. In some embodiments, a subject receives an analgesic. In some embodiments, a subject does not receive an analgesic. In some embodiments, a subject receives an anti-emetic. In some embodiments, a subject does not receive an anti-emetic. In some embodiments, a subject receives electrolyte replacement. In some embodiments, a subject does not receive electrolyte replacement. In some embodiments, a subject receives a tyrosine kinase inhibitor (e.g., a FLT3 inhibitor). In some embodiments, a subject does not receive a tyrosine kinase inhibitor (e.g., a FLT3 inhibitor). In some embodiments, a subject receives prednisone or an equivalent thereof, e.g., at a dose of ≤10 mg/day. In some embodiments, a subject does not receive prednisone or an equivalent thereof. In some embodiments, a subject receives corticosteroid treatment to manage GVHD. In some embodiments, a subject does not receive corticosteroid treatment to manage GVHD. In some embodiments, a subject receives high-dose corticosteroid treatment to manage GVHD. In some embodiments, a subject does not receive high-dose corticosteroid treatment to manage GVHD. In some embodiments, a subject receives corticosteroid treatment to manage, for example, adrenal insufficiency, hypersensitivity reactions, or other non-cancer-related symptoms including premedication for known hypersensitivity reactions to contrast for scans. In some embodiments, a subject does not receive corticosteroid treatment. In some embodiments, a subject receives an immunosuppressive medication. In some embodiments, a subject does not receive an immunosuppressive medication. In some embodiments, a subject receives a donor lymphocyte infusion. In some embodiments, a subject does not receive a donor lymphocyte infusion.

### Conditioning Regimen

Conditioning regimens can be used as part of an alloHCT regimen of the disclosure. Chemotherapy and/or irradiation given soon before a transplant is called a conditioning regimen. Conditioning regimens can help eradicate a patient's disease prior to the infusion of HSPC, suppress immune reactions, and allow a donor HSPC to reconstitute the vacant hematopoietic compartment that results from the conditioning regimen. In some embodiments of the methods of the disclosure, a subject can be treated with myeloablative conditioning prior to infusion of cell populations described herein. In some embodiments of the methods of the disclosure, a subject can be treated with myeloreductive conditioning prior to infusion of cell populations described herein. In some embodiments of the methods of the disclosure, a subject can be treated with a reduced intensity myeloablative conditioning prior to infusion of cell populations described herein. In some embodiments of the methods of the disclosure, a subject can be treated with non-myeloablative conditioning prior to administering a cell population or cell populations described herein.

As used herein, the term conditioning regimen and the like applies to myeloablative conditioning, myeloreductive conditioning, reduced intensity myeloablative therapy/conditioning, and/or non-myeloablative conditioning. As used herein, the term myeloablative therapy/conditioning also includes myeloreductive conditioning and reduced intensity myeloablative conditioning.

In aspects and embodiments, a treatment and/or method further comprises a conditioning regimen, wherein the conditioning regimen is administered before administration of a cell population of the present disclosure (e.g., a population of isolated CD45⁺ cells) that comprises CD34+ cells (e.g., HSPC), Treg, and Tmem, where the cell population is depleted of naïve conventional αβ-T cells or naïve conventional CD3⁺CD25⁻CD45RA⁺ T cells (Tcon). In some embodiments, the conditioning regimen is a myeloablative conditioning regimen. In some cases, the conditioning regimen comprises at least three conditioning reagents, wherein at least one conditioning reagent is thiotepa. In various embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments, the conditioning regimen comprises one or more doses of busulfan, fludarabine, and thiotepa. In embodiments, the one or more doses comprises from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.

In various embodiments, the method further comprises administering a myeloablative conditioning regimen to the patient prior to the administration of any cell population, the conditioning regimen comprising administration of at least one conditioning agent to the subject.

In some embodiments, the patient does not receive any irradiation as part of the myeloablative conditioning regimen.

In embodiments, the at least one conditioning agent is administered from about two to about ten days prior to the administration of any of the cell populations. In some cases, the at least one conditioning agent is administered about five days prior to the administration of any of the cell populations.

In various embodiments, the human subject has undergone myeloablative conditioning regimen before administration of any cell populations and an adverse event is associated with the myeloablative conditioning.

In some embodiments, the at least one conditioning agent comprises thiotepa. In some cases, a dose of thiotepa administered to the patient is in a range of from about 5 to about 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and about 150 mg per meter² body surface area respectively.

In some embodiments, the subject has been conditioned with radiation, chemotherapy, recombinant proteins, antibodies, or toxin-conjugated antibodies, or any combination thereof prior to administering a cell population or cell populations described herein. In some embodiments, the subject is conditioned for cellular graft therapy by first treating the subject with myeloablative therapy. Exemplary myeloablative therapies include chemotherapy or radiotherapy. Myleoablative therapies are thought to provide therapeutic benefit by debulking a tumor and/or reducing the number of cancer cells. Myeloablative regimens eradicate a sufficient number of HSCs that the patient would otherwise increase the chances of a patient developing GVHD. When HSPC are subsequently administered to the myeloablated subject, the donor cells can further attack the cancer and/or and reconstitute the blood and the immune system of the subject.

In some embodiments, the myeloablative therapy comprises administration of thiotepa (TTP), busulfan, cyclophosphamide, Total Body Irradiation (TBI), fludarabine, etoposide, melphalan, anti-thymocyte globulin (ATG), or any combination thereof. In some embodiments, the myeloablative therapy comprises administration an anti-cKIT antibody. In some embodiments, the myeloablative therapy comprises administration an antibody drug conjugate. The antibody drug conjugate can be, for example, anti-CD45-saporin or anti-cKit-saporin therapeutic antibodies. In some embodiments, the myeloablative therapy is a reduced intensity conditioning therapy. Exemplary conditioning regimens are described in Table 11 and Table 12.

A conditioning regimen of this disclosure may comprise one or more doses of busulfan. A conditioning regimen of this disclosure may comprise fludarabine. A conditioning regimen of this disclosure may comprise one or more doses of Cyclophosphamide. A conditioning regimen of this disclosure may comprise one or more doses of Melphalan. A conditioning regimen of this disclosure may comprise one or more doses of Etoposide.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

The conditioning regimen described herein may comprise administration of an alkylating agent such as thiotepa (TTP). A conditioning regimen of this disclosure comprising TTP may comprise at least one more conditioning reagent. The conditioning reagents administered to a subject in addition to TTP may comprise one or more reagents selected from busulfan, dimethyl myleran, prednisone, methyl prednisolone, azathioprine, cyclophosphamide, cyclosparine, monoclonal antibodies against T cells, antilymphocyte globulin and anti-thymocyte globulin, fludarabine, etoposide, radiation, total body irradiation (TBI). Aspects and embodiments include any combination of TTP with the one or more conditioning reagents. In some embodiments, a subject is administered a conditioning regimen comprising thiotepa, busulfan, and fludarabine. In some embodiments, a subject is administered a conditioning regimen comprising thiotepa, fludarabine, and TBI (e.g., HFTBI).

The conditioning regimen described herein may comprise administration of an alkylating agent such as TTP. In some cases, a conditioning regimen of the disclosure may comprise TTP administration on more than one day. A conditioning regimen of the disclosure may comprise administering 2 mg/kg to 14 mg/kg TTP to a subject. A conditioning regimen of this disclosure may comprise administering at least 3 mg/kg TTP to a subject. A conditioning regimen of this disclosure may comprise administering at most 14 mg/kg TTP to a subject. A conditioning regimen of this disclosure may comprise administering 2 mg/kg to 5 mg/kg, 2 mg/kg to 6 mg/kg, 2 mg/kg to 8 mg/kg, 2 mg/kg to 10 mg/kg, 2 mg/kg to 12 mg/kg, 2 mg/kg to 14 mg/kg, 5 mg/kg to 6 mg/kg, 5 mg/kg to 8 mg/kg, 5 mg/kg to 10 mg/kg, 5 mg/kg to 12 mg/kg, 5 mg/kg to 14 mg/kg, 6 mg/kg to 8 mg/kg, 6 mg/kg to 10 mg/kg, 6 mg/kg to 12 mg/kg, 6 mg/kg to 14 mg/kg, 8 mg/kg to 10 mg/kg, 8 mg/kg to 12 mg/kg, 8 mg/kg to 14 mg/kg, 10 mg/kg to 12 mg/kg, 10 mg/kg to 14 mg/kg, or 12 mg/kg to 14 mg/kg TTP to a subject. A conditioning regimen of this disclosure may comprise administering 2 mg/kg, 3 mg/kg, 4 mg/kg, 5 mg/kg, 6 mg/kg, 8 mg/kg, 10 mg/kg, 12 mg/kg, or 14 mg/kg TTP to a subject. A conditioning regimen of this disclosure may comprise administering at most 2 mg/kg, 3 mg/kg, 4 mg/kg, 5 mg/kg, 6 mg/kg, 8 mg/kg, 10 mg/kg or 12 mg/kg TTP to a subject. A conditioning regimen of this disclosure may comprise administering at least 2 mg/kg, 3 mg/kg, 4 mg/kg, 5 mg/kg, 6 mg/kg, 8 mg/kg, 10 mg/kg, 12 mg/kg, or 14 mg/kg TTP to a subject.

As used herein, a recited dose, e.g., #mg/kg, may be relative to a subject's actual body weight (in kg) relative to the subject's ideal body weight (in kg). Or the recited dose may be relative to a subject's adjusted body weight (ABW) if the subject's actual body weight is greater than 120% of the ideal body weight (IBW).

A subject administered one or more cell populations described herein may be administered one or more doses of TTP prior to the cell transplant. A subject receiving one or more cell populations described herein may be administered 1, 2, 3, 4, 5, 6, 7, 8, 9 or 10 doses of TTP prior to the cell transplant. In some cases, each dose of TTP has the same concentration. In some cases, one or more doses of TTP have different concentrations. A subject may be administered 1 mg/kg to 10 mg/kg TTP in a single dose. A subject may be administered at least 1 mg/kg TTP in a single dose. A subject may be administered at most 10 mg/kg TTP in a single dose. A subject may be administered 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 1 mg/kg to 10 mg/kg, 2 mg/kg to 3 mg/kg, 2 mg/kg to 4 mg/kg, 2 mg/kg to 5 mg/kg, 2 mg/kg to 6 mg/kg, 2 mg/kg to 7 mg/kg, 2 mg/kg to 8 mg/kg, 2 mg/kg to 9 mg/kg, 2 mg/kg to 10 mg/kg, 3 mg/kg to 4 mg/kg, 3 mg/kg to 5 mg/kg, 3 mg/kg to 6 mg/kg, 3 mg/kg to 7 mg/kg, 3 mg/kg to 8 mg/kg, 3 mg/kg to 9 mg/kg, 3 mg/kg to 10 mg/kg, 4 mg/kg to 5 mg/kg, 4 mg/kg to 6 mg/kg, 4 mg/kg to 7 mg/kg, 4 mg/kg to 8 mg/kg, 4 mg/kg to 9 mg/kg, 4 mg/kg to 10 mg/kg, 5 mg/kg to 6 mg/kg, 5 mg/kg to 7 mg/kg, 5 mg/kg to 8 mg/kg, 5 mg/kg to 9 mg/kg, 5 mg/kg to 10 mg/kg, 6 mg/kg to 7 mg/kg, 6 mg/kg to 8 mg/kg, 6 mg/kg to 9 mg/kg, 6 mg/kg to 10 mg/kg, 7 mg/kg to 8 mg/kg, 7 mg/kg to 9 mg/kg, 7 mg/kg to 10 mg/kg, 8 mg/kg to 9 mg/kg, 8 mg/kg to 10 mg/kg, or 9 mg/kg to 10 mg/kg TTP in a single dose. A subject may be administered 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at most 1 mg/kg, 2 mg/kg, 3 mg/kg, 4 mg/kg, 5 mg/kg, 6 mg/kg, 7 mg/kg, 8 mg/kg, 9 mg/kg or 10 mg/kg TTP in a single dose. A subject may be administered at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

A conditioning regimen of this disclosure may comprise one or more doses of busulfan. One or more doses of busulfan may be administered to a subject before the administration of one or more doses of another conditioning reagent such as TTP. One or more doses of busulfan may be administered to a subject after the administration of one or more doses of another conditioning reagent such as TTP. One or more doses of busulfan may be administered to a subject along with the administration of one or more doses of another conditioning reagent such as TTP.

A conditioning regimen of this disclosure may comprise administering about 6 mg/kg to about 12 mg/kg busulfan to a subject. A conditioning regimen of this disclosure may comprise administering at least about 6 mg/kg busulfan to a subject. A conditioning regimen of this disclosure may comprise administering at most about 12 mg/kg busulfan to a subject. A conditioning regimen of this disclosure may comprise administering about 6 mg/kg to about 7 mg/kg, about 6 mg/kg to about 8 mg/kg, about 6 mg/kg to about 9 mg/kg, about 6 mg/kg to about 10 mg/kg, about 6 mg/kg to about 11 mg/kg, about 6 mg/kg to about 12 mg/kg, about 7 mg/kg to about 8 mg/kg, about 7 mg/kg to about 9 mg/kg, about 7 mg/kg to about 10 mg/kg, about 7 mg/kg to about 11 mg/kg, about 7 mg/kg to about 12 mg/kg, about 8 mg/kg to about 9 mg/kg, about 8 mg/kg to about 10 mg/kg, about 8 mg/kg to about 11 mg/kg, about 8 mg/kg to about 12 mg/kg, about 9 mg/kg to about 10 mg/kg, about 9 mg/kg to about 11 mg/kg, about 9 mg/kg to about 12 mg/kg, about 10 mg/kg to about 11 mg/kg, about 10 mg/kg to about 12 mg/kg, or about 11 mg/kg to about 12 mg/kg busulfan to a subject. A conditioning regimen of this disclosure may comprise administering 6 mg/kg, 7 mg/kg, 8 mg/kg, 9 mg/kg, 10 mg/kg, 11 mg/kg, or 12 mg/kg busulfan to a subject. A conditioning regimen of this disclosure may comprise administering at least 6 mg/kg, 7 mg/kg, 8 mg/kg, 9 mg/kg, 10 mg/kg or 11 mg/kg busulfan to a subject. A conditioning regimen of this disclosure may comprise administering at most 7 mg/kg, 8 mg/kg, 9 mg/kg, 10 mg/kg, 11 mg/kg, or 12 mg/kg busulfan to a subject.

A subject receiving one or more cell populations described herein may be administered one or more doses of busulfan prior to the cell transplant. A subject receiving one or more cell components described herein may be administered 1, 2, 3, 4, 5, 6, 7, 8, 9 or 10 doses of busulfan prior to the cell transplant. In some cases, each dose of busulfan has the same concentration. In some cases, one or more doses of busulfan have different concentrations. A subject may be administered 1 mg/kg to 10 mg/kg busulfan in a single dose. A subject may be administered at least 1 mg/kg busulfan in a single dose. A subject may be administered at least 2 mg/kg busulfan in a single dose. A subject may be administered at least 3 mg/kg busulfan in a single dose.

A conditioning regimen of this disclosure may comprise one or more doses of fludarabine. One or more doses of fludarabine may be administered to a subject before the administration of one or more doses of another conditioning reagent such as TTP. One or more doses of fludarabine may be administered to a subject after the administration of one or more doses of another conditioning reagent such as TTP. One or more doses of fludarabine may be administered to a subject along with the administration of one or more doses of another conditioning reagent such as TTP.

A conditioning regimen of this disclosure may comprise administering 20 mg/m² to 180 mg/m² fludarabine to a subject based on the surface area of the subject. A conditioning regimen of this disclosure may comprise administering at least 20 mg/m² fludarabine to a subject. A conditioning regimen of this disclosure may comprise administering at most 180 mg/m² fludarabine to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² fludarabine to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² fludarabine to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² fludarabine to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² fludarabine to a subject.

A subject receiving one or more cell components described herein may be administered one or more doses of fludarabine prior to the cell transplant. A subject receiving one or more cell components described herein may be administered 1, 2, 3, 4, 5, 6, 7, 8, 9 or 10 doses of fludarabine prior to the cell transplant. In some cases, each dose of fludarabine has the same concentration. In some cases, one or more doses of fludarabine have different concentrations. A subject may be administered 20 to 60 mg/m² dose of fludarabine in a single dose. A subject may be administered at least 30 mg/m² fludarabine in a single dose. A subject may be administered at least 40 mg/m² fludarabine in a single dose. A subject may be administered at least 50 mg/m² fludarabine in a single dose.

A conditioning regimen of this disclosure may comprise administering 20 mg/m² to 180 mg/m² melphalan to a subject based on the surface area of the subject. A conditioning regimen of this disclosure may comprise administering at least 20 mg/m² melphalan to a subject. A conditioning regimen of this disclosure may comprise administering at most 180 mg/m² melphalan to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² melphalan to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² melphalan to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² melphalan to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² melphalan to a subject.

A subject receiving one or more cell components described herein may be administered one or more doses of melphalan prior to the cell transplant. A subject receiving one or more cell components described herein may be administered 1, 2, 3, 4, 5, 6, 7, 8, 9 or 10 doses of melphalan prior to the cell transplant. In some cases, each dose of melphalan has the same concentration. In some cases, one or more doses of melphalan have different concentrations.

A conditioning regimen of this disclosure may comprise administering 100 mg/kg to 140 mg/kg cyclophosphamide. A conditioning regimen of this disclosure may comprise administering at least 100 mg/kg cyclophosphamide. A conditioning regimen of this disclosure may comprise administering at most 140 mg/kg cyclophosphamide. A conditioning regimen of this disclosure may comprise administering 100 mg/kg to 110 mg/kg, 100 mg/kg to 120 mg/kg, 100 mg/kg to 130 mg/kg, 100 mg/kg to 140 mg/kg, 110 mg/kg to 120 mg/kg, 110 mg/kg to 130 mg/kg, 110 mg/kg to 140 mg/kg, 120 mg/kg to 130 mg/kg, 120 mg/kg to 140 mg/kg, or 130 mg/kg to 140 mg/kg cyclophosphamide. A conditioning regimen of this disclosure may comprise administering about 100 mg/kg, 110 mg/kg, 120 mg/kg, 130 mg/kg, or 140 mg/kg cyclophosphamide. A conditioning regimen of this disclosure may comprise administering at least 100 mg/kg, 110 mg/kg, 120 mg/kg or 130 mg/kg cyclophosphamide. A conditioning regimen of this disclosure may comprise administering at most 110 mg/kg, 120 mg/kg, 130 mg/kg, or 140 mg/kg cyclophosphamide.

A subject receiving one or more cell components described herein may be administered one or more doses of cyclophosphamide prior to the cell transplant. A subject receiving one or more cell components described herein may be administered 1, 2, 3, 4, 5, 6, 7, 8, 9 or 10 doses of cyclophosphamide prior to the cell transplant. In some cases, each dose of cyclophosphamide has the same concentration. In some cases, one or more doses of cyclophosphamide have different concentrations.

A conditioning regimen of this disclosure may comprise administering 40 mg/kg to 80 mg/kg etoposide. A conditioning regimen of this disclosure may comprise administering at least 40 mg/kg etoposide. A conditioning regimen of this disclosure may comprise administering at most 80 mg/kg etoposide. A conditioning regimen of this disclosure may comprise administering 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg etoposide. A conditioning regimen of this disclosure may comprise administering about 40 mg/kg, 50 mg/kg, 60 mg/kg, 70 mg/kg, or 80 mg/kg etoposide. A conditioning regimen of this disclosure may comprise administering at least 40 mg/kg, 50 mg/kg, 60 mg/kg or 70 mg/kg etoposide. A conditioning regimen of this disclosure may comprise administering at most 50 mg/kg, 60 mg/kg, 70 mg/kg, or 80 mg/kg etoposide.

A subject receiving one or more cell components described herein may be administered one or more doses of etoposide prior to the cell transplant. A subject receiving one or more cell components described herein may be administered 1, 2, 3, 4, 5, 6, 7, 8, 9 or 10 doses of etoposide prior to the cell transplant. In some cases, each dose of etoposide has the same concentration. In some cases, one or more doses of etoposide have different concentrations.

A conditioning regimen of this disclosure comprising TTP may comprise one or more doses of total body irradiation (TBI) such as hyperfractionated TBI (HFTBI). One or more doses of HFTBI may be administered to a subject before the administration of one or more doses of another conditioning reagent such as TTP. One or more doses of HFTBI may be administered to a subject after the administration of one or more doses of another conditioning reagent such as TTP. One or more doses of HFTBI may be administered to a subject along with the administration of one or more doses of another conditioning reagent such as TTP.

A conditioning regimen of this disclosure may comprise administering 500 cGy to 1,600 cGy TBI to a subject. A conditioning regimen of this disclosure may comprise administering 800 cGy to 1,500 cGy TBI to a subject. A conditioning regimen of this disclosure may comprise administering at least 500 cGy TBI to a subject. A conditioning regimen of this disclosure may comprise administering at least 800 cGy TBI to a subject. A conditioning regimen of this disclosure may comprise administering at most 1,500 cGy TBI to a subject. A conditioning regimen of this disclosure may comprise administering at most 1,600 cGy TBI to a subject. A conditioning regimen of this disclosure may comprise administering 800 cGy to 900 cGy, 800 cGy to 1,000 cGy, 800 cGy to 1,100 cGy, 800 cGy to 1,200 cGy, 800 cGy to 1,300 cGy, 800 cGy to 1,375 cGy, 800 cGy to 1,400 cGy, 800 cGy to 1,500 cGy, 900 cGy to 1,000 cGy, 900 cGy to 1,100 cGy, 900 cGy to 1,200 cGy, 900 cGy to 1,300 cGy, 900 cGy to 1,375 cGy, 900 cGy to 1,400 cGy, 900 cGy to 1,500 cGy, 1,000 cGy to 1,100 cGy, 1,000 cGy to 1,200 cGy, 1,000 cGy to 1,300 cGy, 1,000 cGy to 1,375 cGy, 1,000 cGy to 1,400 cGy, 1,000 cGy to 1,500 cGy, 1,100 cGy to 1,200 cGy, 1,100 cGy to 1,300 cGy, 1,100 cGy to 1,375 cGy, 1,100 cGy to 1,400 cGy, 1,100 cGy to 1,500 cGy, 1,200 cGy to 1,300 cGy, 1,200 cGy to 1,375 cGy, 1,200 cGy to 1,400 cGy, 1,200 cGy to 1,500 cGy, 1,300 cGy to 1,375 cGy, 1,300 cGy to 1,400 cGy, 1,300 cGy to 1,500 cGy, 1,375 cGy to 1,400 cGy, 1,375 cGy to 1,500 cGy, or 1,400 cGy to 1,500 cGy TBI to a subject. A conditioning regimen of this disclosure may comprise administering about 500 cGy, 600 cGy, 700 cGy, 800 cGy, 900 cGy, 1,000 cGy, 1,025 cGy, 1,050 cGy, 1,075 cGy, 1,100 cGy, 1,125 cGy, 1,150 cGy, 1,175 cGy, 1,200 cGy, 1,225 cGy, 1,250 cGy, 1,275 cGy, 1,300 cGy, 1,325 cGy, 1,350 cGy, 1,375 cGy, 1,400 cGy, 1,425 cGy, 1,450 cGy, 1,475 cGy, 1,500 cGy, 1,525 cGy, 1,550 cGy, 1,575 cGy, or 1,600 cGy TBI to a subject. A conditioning regimen of this disclosure may comprise administering at least 500 cGy, 600 cGy, 700 cGy, 800 cGy, 900 cGy, 1,000 cGy, 1,025 cGy, 1,050 cGy, 1,075 cGy, 1,100 cGy, 1,125 cGy, 1,150 cGy, 1,175 cGy, 1,200 cGy, 1,225 cGy, 1,250 cGy, 1,275 cGy, 1,300 cGy, 1,325 cGy, 1,350 cGy, 1,375 cGy, 1,400 cGy, 1,425 cGy, 1,450 cGy, 1,475 cGy, 1,500 cGy, 1,525 cGy, 1,550 cGy, 1,575 cGy, or 1,600 cGy TBI to a subject. A conditioning regimen of this disclosure may comprise administering at most 500 cGy, 600 cGy, 700 cGy, 800 cGy, 900 cGy, 1,000 cGy, 1,025 cGy, 1,050 cGy, 1,075 cGy, 1,100 cGy, 1,125 cGy, 1,150 cGy, 1,175 cGy, 1,200 cGy, 1,225 cGy, 1,250 cGy, 1,275 cGy, 1,300 cGy, 1,325 cGy, 1,350 cGy, 1,375 cGy, 1,400 cGy, 1,425 cGy, 1,450 cGy, 1,475 cGy, 1,500 cGy, 1,525 cGy, 1,550 cGy, 1,575 cGy, or 1,600 cGy TBI to a subject.

A subject receiving one or more cell components described herein may be administered one or more doses of TBI, e.g., fractionated TBI, prior to the cell transplant. A subject receiving one or more cell components described herein may be administered 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more doses of, e.g., fractionated, total body irradiation (TBI) prior to the cell transplant. In some cases, each dose of TBI (e.g., fractionated TBI) has the same concentration. In some cases, one or more doses of TBI (e.g., fractionated TBI) have different concentrations. A subject may be administered a 75 to 150 cGys of fractionated TBI in a single dose. A subject may be administered at least 75 cGys fractionated TBI in a single dose. A subject may be administered at least 100 cGys fractionated TBI in a single dose. A subject may be administered at least 125 cGys fractionated TBI in a single dose.

A subject may receive the one or more cell populations described herein 1 day after completing a conditioning regimen. A subject may receive the one or more cell components described herein 2, 3, 4, 5, 6, 7, 8, 9, 10 days after completing a conditioning regimen. A subject may receive the one or more cell components described herein 1 day after receiving a final dose of a conditioning reagent such as TTP. A subject may receive the one or more cell components described herein 2, 3, 4, 5, 6, 7, 8, 9 or 10 days after receiving a final dose of a conditioning reagent such as TTP. A subject may receive the one or more cell components described herein 1 day after receiving a first dose of a conditioning reagent such as TTP. A subject may receive the one or more cell components described herein 2, 3, 4, 5, 6, 7, 8, 9 or 10 days after receiving a first dose of a conditioning reagent such as TTP.

### Graft Versus Host Disease (GVHD)

Graft versus host disease (GVHD) is a significant cause of morbidity and mortality in hematopoietic stem cell transplantation (HCT) recipients. Aspects and embodiments herein provide compositions and methods that reduce the incidence of GVHD, reduce the severity of GVHD, reduce the relative risk of GVHD, prevent GVHD, or a combination thereof in HCT recipients.

Graft-versus-host disease (GVHD) is an inflammatory disease that can occur in the allogeneic transplant setting. GVHD involves donor cells (graft) attacking recipient cells (host). GVHD can be life-threatening and can involve, for example, the skin, the intestines, and/or the liver. The morbidity and mortality associated GVHD can be a major factor limiting the success of HCT. GVHD can occur despite use of a HLA-matched sibling donor, and the use of various GVHD prophylactic/immunosuppressive agents, for example, use of two or more of tacrolimus, sirolimus, cyclosporine, methotrexate, mycophenolate, mycophenolate mofetil (MMF), anti-thymocyte globulin and corticosteroids.

### GVHD Classification and Grading

GVHD can be classified into acute GVHD (aGVHD) and chronic GVHD (cGVHD). In some embodiments, GVHD that occurs within the first 100 days post-transplant can be referred to as aGVHD, and GVHD after the first 100 days can be referred to as chronic GVHD (cGVHD). cGVHD is a major source of late treatment-related complications, and can be life-threatening. In addition to inflammation, cGVHD can lead to the development of fibrosis, which can result in functional disability.

Yet another aspect provides any herein-disclosed a pharmaceutical treatment in which a risk and/or severity of an adverse event associated with the pharmaceutical treatment is reduced as compared to a similar pharmaceutical treatment in which a human subject receives alloHCT but does not receive cell populations enriched in Treg or is any herein-disclosed method in which a risk and/or severity of an adverse event associated with the method is reduced as compared to a similar method in which a human subject receives alloHCT but does not receive cell populations enriched in Treg.

In various embodiments, the adverse event is acute GVHD (aGVHD). In some embodiments, the adverse event is stage two or greater aGVHD. In embodiments, the adverse event is chronic GVHD (cGVHD). In various embodiments, the human subject has no cGVHD about one year after being administered the cell populations. In some embodiments, the adverse event is moderate to severe cGVHD. In embodiments, the adverse event is acute graft vs host disease (aGVHD), e.g., stage two or greater aGVHD. In some cases, the patient has no stage two or higher aGVHD about 180 days after being administered the cell populations. In various embodiments, the adverse event is chronic graft vs host disease (cGVHD). In some cases, the patient has no cGVHD about one year after being administered the cell populations. some embodiments, the adverse event is moderate to severe cGVHD. In some cases, the patient does not have moderate to severe cGVHD about one year after being administered the cell populations.

In embodiments, a patient does not develop GVHD within about 30 days of administration of a cell population of the present disclosure (e.g., a population of isolated CD45⁺ cells), does not develop GVHD within about 100 days of administration of a cell population of the present disclosure (e.g., a population of isolated CD45+ cells), does not develop GVHD within about 180 days of administration of a cell population of the present disclosure (e.g., a population of isolated CD45+ cells), and/or does not develop GVHD within about one year of administration of a cell population of the present disclosure (e.g., a population of isolated CD45+ cells).

Acute GVHD (aGVHD) and chronic GVHiD (cGVHD) can be graded using a system that first evaluates GVHD stages for the skin, liver, and gut, and then combines scoring from the organ staging to determine an overall GVHD grade. An example of a GVHD staging criteria that can be used for individual organs are provided in TABLE 1.

TABLE 1

| Organ | Stage | Description |
|---|---|---|
| Skin | 0 | No active (erythematous) GVHD rash |
| | 1 | Maculo-papular eruption over <25% of body area |
| | 2 | Maculo-papular eruption over >25 to 50% of body area |
| | 3 | Generalized erythroderma/maculo-papular eruption over >50% of body area |
| | 4 | Generalized erythroderma/maculo-papular eruption over >50% of body area |
| | | plus bullous formation and desquamation over >5% of body area |
| Liver | 0 | Bilirubin <2 mg/dL |
| | 1 | Bilirubin 2.0 to 3.0 mg/dL; |
| | | serum glutamic oxaloacetic transaminase (SGOT) SGOT 150 to 750IU |
| | 2 | Bilirubin 3.1 to 6.0 mg/dL |
| | 3 | Bilirubin 6.1 to 15.0 mg/dL |
| | 4 | Bilirubin >15.0 mg/dL |
| Gut | 0 | <500 mL/day or <3 episodes/day |
| | 1 | Diarrhea >30 mL/kg or >500 mL/day |
| | 2 | Diarrhea >60 mL/kg or >1000 mL/day |
| | 3 | Diarrhea >90 mL/kg or >1500 mL/day |
| | 4 | Diarrhea >90 mL/kg or >2000 mL/day; or severe abdominal pain with or |
| | | without ileus |

TABLE 2 provides one set of criteria for assessing overall GVHD grade.

TABLE 2

| | | | | | ECOG |
|---|---|---|---|---|---|
| Grade | Skin | Liver | | Gut | performance |
| 1 | 1 to 2 | 0 | | 0 | 0 |
| 2 | 1 to 3 | 1 | and/or | 1 | 0-1 |
| 3 | 2 to 3 | 2 to 4 | and/or | 2 to 3 | 2-3 |
| 4 | 2 to 4 | 2 to 4 | and/or | 2 to 3 | 3-4 |

aGVHD grade can also be determined based on most severe target organ involvement as defined in the MAGIC standardization criteria described by Harris et al., “International, multicenter standardization of acute graft-versus-host disease clinical data collection: a report from the Mount Sinai Acute GVHD International Consortium.” Biology of Blood and Marrow Transplantation 22.1 (2016): 4-10. For example, aGVHD organ staging can be evaluated according to TABLE 3, and overall aGVHD grade can be assessed as follows:
Grade 0: No Stage 1-4 of any organ.
Grade I: Stage 1-2 skin without liver, upper GI, or lower GI involvement.
Grade II: Stage 3 rash and/or Stage 1 liver and/or Stage 1 upper GI and/or Stage 1 lower GI.
Grade III: Stage 2-3 liver and/or Stage 2-3 lower GI, with Stage 0-3 skin and/or Stage 0-1 upper GI. Grade IV: Stage 4 skin, liver, or lower GI involvement, with Stage 0-1 upper GI.

TABLE 3

| | Skin (Active | Liver | | | |
|---|---|---|---|---|---|
| Stage | Erythema Only) | (Bilirubin) | | Upper GI | Lower GI (stool output/day) |
| 0 | No active | <2 | mg/dL | No or intermittent | Adult: <500 mL/day |
| | (erythematous) | | | nausea, vomiting or | or <3 episodes/day |
| | GvHD rash | | | anorexia | Child: <10 mL/kg/day |
| | | | | | or <4 episodes/day |
| 1 | Maculopapular | 2-3 | mg/dL | Persistent nausea, | Adult: 500-999 mL/day or 3-4 |
| | rash <25% BSA | | | vomiting or | episodes/day |
| | | | | anorexia | Child: 10-19.9 mL/kg/day or 5-7 |
| | | | | | episodes/day |
| 2 | Maculopapular rash | 3.1-6 | mg/dL | | Adult: 1000-1500 mL/day or 57 |
| | 25-50% BSA | | | | episodes/day |
| | | | | | Child: 20-30 mL/kg/day or 7-10 |
| | | | | | episodes/day |
| 3 | Maculopapular | 6.1-15 | mg/dL | | Adult: >1500 mL/day |
| | rash >50% BSA | | | | or >7 episodes/day |
| | | | | | Child: >30 mL/kg/day |
| | | | | | or >10 episodes/day |
| 4 | Generalized | >15 | mg/dL | | Severe abdominal pain with or |
| | erythroderma | | | | without ileus or grossly bloody |
| | (>50% BSA) plus | | | | stool (regardless of stool volume) |
| | bullous formation | | | | |
| | and desquamation >5% | | | | |
| | BSA | | | | |

In some embodiments, GVHD stage and GVHD grade are synonyms.

cGVHD can also be assessed by the method described by Jagasia et al., “National Institutes of Health consensus development project on criteria for clinical trials in chronic graft-versus-host disease: I. The 2014 diagnosis and staging working group report.” Biology of Blood and Marrow Transplantation 21.3 (2015): 389-401. To establish a diagnosis of cGVHD, these criteria can require, for example, at least one diagnostic manifestation of chronic GVHD or at least one distinctive manifestation plus a pertinent biopsy, laboratory, or other test (e.g., PFTs, Schirmer's test), evaluation by a specialist (ophthalmologist, gynecologist) or radiographic imaging showing chronic GVHD in the same or another organ.

Organ systems can be scored as described in Jagasia et al., and Mild cGVHD can be present when one or two organs are involved with no more than score 1, plus a lung score of zero; moderate cGVHD can be present when three or more organs are involved with no more than score 1, or when at least one non-lung organ has a score of 2, or when the lungs have a score of 1; and severe cGVHD can be present when at least one organ has a score of 4, or the lungs have a score of 2 or 3.

TABLE 4 provides diagnostic and distinctive manifestations of cGVHD. infection, drug effect, malignancy, or other causes are excluded for distinctive manifestations. Bronchiolitis obliterans syndrome can be diagnostic for lung chronic GVHD only if distinctive sign or symptom present in another organ. Diagnosis of chronic GVHD based on myositis or polymyositis can require a biopsy.

TABLE 4

| Organ or | Diagnostic (Sufficient to Establish | Distinctive¹ (Seen in chronic GVHD, but |
|---|---|---|
| Site | the Diagnosis of chronic GVHD) | Insufficient Alone to Establish a Diagnosis) |
| Skin | Poikiloderma Lichen planus-like | Depigmentation Papulosquamous |
| | features | lesions |
| | Sclerotic features Morphea-like | |
| | features | |
| | Lichen sclerosus-like features | |
| Nails | | Dystrophy |
| | | Longitudinal ridging, splitting or brittle features |
| | | Onycholysis |
| | | Pterygium unguis |
| | | Nail loss (usually symmetric, affects most nails) |
| Scalp and | | New onset of scarring or nonscarring scalp |
| body hair | | alopecia (after recovery from chemoradiotherapy) |
| | | Loss of body hair |
| Mouth | Lichen planus-like changes | Xerostomia Mucoceles Mucosal |
| | | atrophy Ulcers |
| | | Pseudomembranes |
| Eyes | | New onset dry, gritty, or painful eyes |
| | | Cicatricial conjunctivitis |
| | | Keratoconjunctivitis sicca |
| | | Confluent areas of punctate keratopathy |
| Genitalia | Lichen planus-like features Lichen | Erosions Fissures |
| Females | sclerosus-like features | |
| Males | Vaginal scarring or clitoral/labial | Ulcers |
| | agglutination | |
| | Phimosis or urethral/meatus scarring or | |
| | stenosis | |
| GI Tract | Esophageal web Strictures or stenosis | |
| | in the upper to | |
| | mid third of the esophagus | |
| Lung | Bronchiolitis obliterans diagnosed | Air trapping and bronchiectasis on chest CT |
| | with lung biopsy | |
| | Bronchiolitis obliterans syndrome² | |
| Muscles, | Joint stiffness or contractures | Myositis or polymyositis³ |
| fascia, joints | secondary to fasciitis or sclerosis | |

In some embodiments, GVHD severity can be graded using the Glucksberg grade (I-IV) or the International Bone Marrow Transplant Registry (IBMTR) grading system (A-D). The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade.

### GVHD Prophylaxis and Treatment

Immunosuppressive agents can be used to reduce the likelihood of GVHD (GVHD prophylactic agents), or to treat GVHD once it occurs (GVHD therapeutic agents). However, in some cases, the use of GVHD prophylactic agents, GVHD therapeutic agents, or both can be insufficient to effectively prevent or treat GVHD. For example, the incidence of GVHD in graft recipients can be high despite use of use of tacrolimus, sirolimus, cyclosporine, methotrexate, mycophenolate, anti-thymocyte globulin, corticosteroids, or a combination thereof (e.g., two or more of the agents).

Administering multiple GVHD prophylactic and/or therapeutic agents, high doses of GVHD prophylactic and/or therapeutic agents, or both can fail to effectively treat GVHD in many alloHCT settings or can result in increased susceptibility to infection and decreased graft versus tumor therapeutic effects.

Non-limiting examples of GVHD prophylactic and/or GVHD therapeutic agents that can be used include calcineurin inhibitors (e.g., tacrolimus, cyclosporine A), sirolimus, monoclonal antibodies, methotrexate, mycophenolate, anti-thymocyte globulin, corticosteroids, azathioprine, and mycophenolate mofetil. Monoclonal antibodies useful as immunosuppressive agents include, for example, antagonist antibodies, (e.g., antibodies that antagonize IL-2R such as basiliximab and daclizumab), and antibodies that deplete an immune cell population by antibody dependent cellular cytotoxicity.

Compositions and methods described herein may comprise administering one or more GVHD prophylactic agents to an HCT recipient. GVHD prophylaxis in such cases should be considered different from GVHD treatment such that the GVHD prophylactic agent will be administered to the HCT recipient before an incidence of GVHD is assessed. In some cases, an HCT recipient may be administered one or more GVHD prophylactic agents but not a GVHD therapeutic agent. In some cases, a HCT recipient does not require treatment for GVHD and/or does not receive treatment for GVHD.

Compositions and methods disclosed herein can reduce the incidence of GVHD, reduce the severity of GVHD, reduce the relative risk of GVHD, prevent GVHD, or a combination thereof in HCT recipients. In some embodiments, such benefits are achieved despite administering no GVHD prophylactic agents as disclosed herein. In some embodiments, such benefits are achieved despite administering a reduced number of GVHD prophylactic agents (e.g., a single GVHD prophylactic agents), a low dose of GVHD prophylactic agent(s), or a combination thereof as disclosed herein. In some embodiments, 1 GVHD prophylactic agent is administered to a subject. In some embodiments, no more than GVHD prophylactic agent is administered to a subject. In some embodiments, 2 GVHD prophylactic agents are administered to a subject. In some embodiments, no more than 2 GVHD prophylactic agents are administered to a subject. In some embodiments, no GVHD prophylactic agents are administered to a subject.

In some embodiments, one or more GVHD prophylactic agents may be administered to a HCT recipient for a duration of 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 18 months, 24 months, 36 months post-transplant of one or more cell populations. One or more GVHD prophylactic agents may be administered to an HCT recipient starting the day of transplant of cell populations. For instance, a GVHD prophylactic regimen may begin the day an HSPC cell population and/or a Treg cell population is administered to the recipient.

In some embodiments, tacrolimus is not administered to a subject. In some embodiments, sirolimus is not administered to a subject. In some embodiments, cyclosporine is not administered to a subject. In some embodiments, methotrexate is not administered to a subject. In some embodiments, mycophenolate is not administered to a subject. In some embodiments, anti-thymocyte globulin is not administered to a subject. In some embodiments, corticosteroids are not administered to a subject.

In some embodiments, the GVHD prophylactic agent is tacrolimus.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from about 12 to about 24 hours after administration of the cells. In some cases, the tacrolimus GHVDPA is administered for a period of time up to about 90 days, is administered for a period of time up to about 60 days. In some embodiments, the tacrolimus GHVDPA is initially administered to the patient at about 0.03 mg/kg patient's actual or ideal body weight/day. In some cases, a dose of the tacrolimus GVHDPA administered to the patient is tapered starting at about 90 days after a first dose is administered to the patient or is tapered starting at about 45 days after a first dose is administered to the patient.

Aspects and embodiments herein provide a method of transplanting cell populations into a human patient as a part of a treatment regimen for a hematologic malignancy. The method comprises administering to the patient a population of hematopoietic stem and progenitor cells (HSPC; the population of HSPC comprising HSPC and a liquid suspending the HSPC; administering to the patient a population of regulatory T cells (Treg) to be administered to the patient, the population of Treg comprising Treg and a liquid suspending the Treg; administering to the patient a population of memory T cells (Tmem) to be administered to the patient, the population of Tmem comprising Tmem and a liquid suspending the Tmem; and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In cases where aGVHD occurs, responsiveness of aGVHD to GVHD therapeutic agents (e.g., corticosteroids) can be assessed by the criteria of TABLE 5.

TABLE 5

| | |
|---|---|
| Complete response | Complete resolution of acute GvHD symptoms in all organs, without |
| (CR) | secondary GvHD therapy |
| Partial response (PR) | Improvement in GvHD stage in all initial GvHD target organs without |
| | complete resolution and without worsening in any other GvHD target |
| | organs, without secondary GvHD therapy |
| Very good partial | Improvement in GvHD in all initial GvHD target organs, with maximum |
| response (VGPR) | Stage I involvement in one or more organs (except upper gastrointestinal |
| | tract), without secondary GvHD therapy |
| No response (NR) | Same grade of GvHD or progression of GvHD in any organ or death, or the |
| | addition of secondary GvHD therapy |
| Progression | Worsening GvHD in at least 1 organ with or without amelioration in any |
| | organ |

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a very good partial response to GVHD therapeutic agents.

In cases where cGVHD occurs, responsiveness of cGVHD to GVHD therapeutic agents (e.g., corticosteroids) can be assessed by the criteria of TABLE 6. Abbreviations used: ALT, alanine transaminase; FEV1, forced expiratory volume in the first second; OMRS, Oral Mucosa Rating Scale; PFTs, pulmonary function tests; P-ROM, photographic range of motion; ULN, upper limit of normal.

TABLE 6

| Organ | Complete Response | Partial Response | Progression |
|---|---|---|---|
| Skin | NIH Skin Score 0 after | Decrease in NIH Skin | Increase in NIH Skin Score by 1 |
| | previous involvement | Score by 1 or more points | or more points, except 0 to 1 |
| Eyes | NIH Eye Score 0 after | Decrease in NIH Eye Score | Increase in NIH Eye Score by 1 |
| | previous involvement | by 1 or more points | or more points, except 0 to 1 |
| Mouth | NIH Modified OMRS 0 | Decrease in NIH Modified | Increase in NIH Modified |
| | after previous involvement | OMRS of 2 or more points | OMRS of 2 or more points |
| Esophagus | NIH Esophagus Score 0 | Decrease in NIH | Increase in NIH Esophagus |
| | after previous involvement | Esophagus Score by 1 or | Score by 1 or more points, |
| | | more points | except 0 to 1 |
| Upper GI | NIH Upper GI Score 0 after | Decrease in NIH Upper GI | Increase in NIH Upper GI |
| | previous involvement | Score by 1 or more points | Score by 1 or more points, |
| | | | except 0 to 1 |
| Lower GI | NIH Lower GI Score 0 after | Decrease in NIH Lower GI | Increase in NIH Lower GI |
| | previous involvement | Score by 1 or more points | Score by 1 or more points, |
| | | | except from 0 to 1 |
| Liver | Normal ALT, alkaline | Decrease by 50% | Increase by 2 × ULN |
| | phosphatase, and Total | | |
| | bilirubin after previous | | |
| | elevation of 1 or more | | |
| Lungs | Normal % FEV1 after | Increase by 10% | Decrease by 10% predicted |
| | previous involvement | predicted absolute value | absolute value of % FEV1 |
| | If PFTs not available, | of % FEV1 | If PFTs not available, |
| | NIH Lung Symptom | If PFTs not available, | increase in NIH Lung - |
| | Score 0 after previous | decrease in NIH Lung | Symptom Score by 1 or |
| | involvement | Symptom Score by 1 or | more points, except 0 to 1 |
| | | more points | |
| Joints and | Both NIH Joint and Fascia | Decrease in NIH Joint and | Increase in NIH Joint and Fascia |
| fascia | Score 0 and P-ROM score 25 | Fascia Score by 1 or more | Score by 1 or more points or |
| | after previous involvement by | points or increase in P-ROM | decrease in P-ROM score by 1 |
| | at least 1 measure | score by 1 point for any site | point for any site |
| Global | Clinician overall severity | Clinician overall severity | Clinician overall severity score |
| | score 0 | score decreases by 2 or more | increases by 2 or more points on |
| | | points on a 0-10 scale | a 0-10 scale |

In some embodiments, in cases where cGVHD occurs in subjects that receive a composition of the disclosure, at least about 40%, at least about 500%, at least about 550%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where cGVHD occurs in subjects that receive a composition of the disclosure, at least about 40%, at least about 50%, at least about 550%, at least about 60%, at least about 65%, at least about 70%, at least about 75%, at least about 80%, at least about 85%, at least about 90%, or at least about 9500 of cases exhibit a partial response to GVHD therapeutic agents.

### Acute GVHD Incidence

Subjects administered a composition of the disclosure (e.g., a cell population as described herein) may exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 5000, less than about 5500 less than about 60%, less than about 65%, less than about 70%, less than about 750%, less than about 800, or less than about 85% of subjects that are administered a composition of the disclosure develop ≥grade 1 aGVHD, for example, within 30 days post-transplant, 100 days post-transplant, or within another suitable amount of time post-transplant as disclosed herein.

Subjects administered a composition of the disclosure (e.g., a population of isolated CD45⁺ cells) may exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 2 aGVHD. In some embodiments, less than about 15% of subjects that are administered a composition of the disclosure develop ≥grade 2 aGVHD. In some embodiments, less than about 10% of subjects that are administered a composition of the disclosure develop ≥grade 2 aGVHD. In some embodiments, less than about 8% of subjects that are administered a composition of the disclosure develop ≥grade 2 aGVHD. In some embodiments, less than about 7% of subjects that are administered a composition of the disclosure develop ≥grade 2 aGVHD.

Subjects administered a composition of the disclosure (e.g., a cell population as described herein) may exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 3 aGVHD. In some embodiments, less than about 15% of subjects that are administered a composition of the disclosure develop ≥grade 3 aGVHD. In some embodiments, less than about 10% of subjects that are administered a composition of the disclosure develop ≥grade 3 aGVHD. In some embodiments, less than about 5% of subjects that are administered a composition of the disclosure develop ≥grade 3 aGVHD. In some embodiments, less than about 3% of subjects that are administered a composition of the disclosure develop ≥grade 3 aGVHD. In some embodiments, less than about 2% of subjects that are administered a composition of the disclosure develop ≥grade 3 aGVHD. In some embodiments, less than about 1% of subjects that are administered a composition of the disclosure develop ≥grade 3 aGVHD.

Subjects administered a composition of the disclosure (e.g., a cell population as described herein) may exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure develop ≥grade 4 aGVHD. In some embodiments, less than about 5% of subjects that are administered a composition of the disclosure develop ≥grade 4 aGVHD. In some embodiments, less than about 3% of subjects that are administered a composition of the disclosure develop ≥grade 4 aGVHD. In some embodiments, less than about 2% of subjects that are administered a composition of the disclosure develop ≥grade 4 aGVHD. In some embodiments, less than about 1% of subjects that are administered a composition of the disclosure develop ≥grade 4 aGVHD.

The incidence of aGVHD can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days post-transplant.

The incidence of aGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population of isolated CD45⁺ cells) have a decreased incidence, severity, timing, or any combination thereof of Grade I to Grade IV acute GVHD (aGVHD), relative to a corresponding human subject that has been administered a standard myeloablative allogeneic hematopoietic stem cell transplant (alloHSCT). In some embodiments, the subject is free of Grade I to Grade IV aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, subjects administered a composition of the disclosure (e.g., a cell population of isolated CD45⁺ cells) have increased aGVHD survival, relative to a corresponding human subject that has been administered a standard myeloablative allogeneic hematopoietic stem cell transplant (alloHSCT). In some embodiments, the subject experiences aGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, subjects administered a composition of the disclosure (e.g., a cell population of isolated CD45⁺ cells) have decreased incidence of steroid-refractory aGVHD, relative to a corresponding human subject that has been administered a standard myeloablative allogeneic hematopoietic stem cell transplant (alloHSCT). In some embodiments, the subject is free of steroid-refractory aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

### No GVHD Prophylaxis

Subjects administered a composition of the disclosure (e.g., a cell population as described herein) in the absence of GVHD prophylactic agents may exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) in the absence of GVHD prophylactic agents may exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 aGVHD, for example, within 30 days post-transplant, 100 days post-transplant, or within another suitable amount of time post-transplant as disclosed herein.

In some embodiments, less than about 50% of subjects that are administered a composition of the disclosure in the absence of GVHD prophylactic agents develop ≥grade 2 aGVHD. In some embodiments, less than about 40% of subjects that are administered a composition of the disclosure in the absence of GVHD prophylactic agents develop ≥grade 2 aGVHD. In some embodiments, less than about 30% of subjects that are administered a composition of the disclosure in the absence of GVHD prophylactic agents develop ≥grade 2 aGVHD. In some embodiments, less than about 25% of subjects that are administered a composition of the disclosure in the absence of GVHD prophylactic agents develop ≥grade 2 aGVHD. In some embodiments, less than about 20% of subjects that are administered a composition of the disclosure in the absence of GVHD prophylactic agents develop ≥grade 2 aGVHD. In some embodiments, less than about 10% of subjects that are administered a composition of the disclosure in the absence of GVHD prophylactic agents develop ≥grade 2 aGVHD.

Subjects administered a composition of the disclosure (e.g., a cell population as described herein) in the absence of GVHD prophylactic agents may exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 aGVHD, for example, within 30 days post-transplant, 100 days post-transplant, or within another suitable amount of time post-transplant as disclosed herein.

In some embodiments, less than about 40% of subjects that are administered a composition of the disclosure in the absence of GVHD prophylactic agents develop ≥grade 3 aGVHD. In some embodiments, less than about 30% of subjects that are administered a composition of the disclosure in the absence of GVHD prophylactic agents develop ≥grade 3 aGVHD. In some embodiments, less than about 20% of subjects that are administered a composition of the disclosure in the absence of GVHD prophylactic agents develop ≥grade 3 aGVHD. In some embodiments, less than about 10% of subjects that are administered a composition of the disclosure in the absence of GVHD prophylactic agents develop ≥grade 3 aGVHD. In some embodiments, less than about 5% of subjects that are administered a composition of the disclosure in the absence of GVHD prophylactic agents develop ≥grade 3 aGVHD. In some embodiments, less than about 3% of subjects that are administered a composition of the disclosure in the absence of GVHD prophylactic agents develop ≥grade 3 aGVHD. In some embodiments, less than about 2% of subjects that are administered a composition of the disclosure in the absence of GVHD prophylactic agents develop ≥grade 3 aGVHD. In some embodiments, less than about 1% of subjects that are administered a composition of the disclosure in the absence of GVHD prophylactic agents develop ≥grade 3 aGVHD.

Subjects administered a composition of the disclosure (e.g., a cell population as described herein) in the absence of GVHD prophylactic agents may exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure in the absence of GVHD prophylactic agents develop ≥grade 4 aGVHD, for example, within 30 days post-transplant, 100 days post-transplant, or within another suitable amount of time post-transplant as disclosed herein.

In some embodiments, less than about 20% of subjects that are administered a composition of the disclosure in the absence of GVHD prophylactic agents develop ≥grade 4 aGVHD. In some embodiments, less than about 15% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD. In some embodiments, less than about 5% of subjects that are administered a composition of the disclosure in the absence of GVHD prophylactic agents develop ≥grade 4 aGVHD. In some embodiments, less than about 3% of subjects that are administered a composition of the disclosure in the absence of GVHD prophylactic agents develop ≥grade 4 aGVHD. In some embodiments, less than about 2% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD.

The absence of GVHD prophylactic agents can refer to cases where no GVHD prophylactic agents are administered to the subject for the first 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 105 days, 110 days, 112 days, 115 days, 119 days, or 120 days post-transplant.

### Single Agent GVHD Prophylaxis

Subjects administered a composition of the disclosure (e.g., a cell population as described herein) and no more than one GVHD prophylactic agent (for example, a single GVHD prophylactic agent) may exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) may exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 2 aGVHD. In some embodiments, less than about 15% of subjects that are administered a composition of the disclosure and no more than one GVHD prophylactic agent (for example, a single GVHD prophylactic agent) develop ≥grade 2 aGVHD. In some embodiments, less than about 10% of subjects that are administered a composition of the disclosure and no more than one GVHD prophylactic agent (for example, a single GVHD prophylactic agent) develop ≥grade 2 aGVHD. In some embodiments, less than about 8% of subjects that are administered a composition of the disclosure and no more than one GVHD prophylactic agent (for example, a single GVHD prophylactic agent) develop ≥grade 2 aGVHD. In some embodiments, less than about 7% of subjects that are administered a composition of the disclosure and no more than one GVHD prophylactic agent (for example, a single GVHD prophylactic agent) develop ≥grade 2 aGVHD.

Subjects administered a composition of the disclosure (e.g., a cell population as described herein) and no more than one GVHD prophylactic agent (for example, a single GVHD prophylactic agent) may exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 3 aGVHD. In some embodiments, less than about 15% of subjects that are administered a composition of the disclosure and no more than one GVHD prophylactic agent (for example, a single GVHD prophylactic agent) develop ≥grade 3 aGVHD. In some embodiments, less than about 10% of subjects that are administered a composition of the disclosure and no more than one GVHD prophylactic agent (for example, a single GVHD prophylactic agent) develop ≥grade 3 aGVHD. In some embodiments, less than about 5% of subjects that are administered a composition of the disclosure and no more than one GVHD prophylactic agent (for example, a single GVHD prophylactic agent) develop ≥grade 3 aGVHD. In some embodiments, less than about 3% of subjects that are administered a composition of the disclosure and no more than one GVHD prophylactic agent (for example, a single GVHD prophylactic agent) develop ≥grade 3 aGVHD. In some embodiments, less than about 2% of subjects that are administered a composition of the disclosure and no more than one GVHD prophylactic agent (for example, a single GVHD prophylactic agent) develop ≥grade 3 aGVHD. In some embodiments, less than about 1% of subjects that are administered a composition of the disclosure and no more than one GVHD prophylactic agent (for example, a single GVHD prophylactic agent) develop ≥grade 3 aGVHD.

Subjects administered a composition of the disclosure (e.g., a cell population as described herein) and no more than one GVHD prophylactic agent (for example, a single GVHD prophylactic agent) may exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and no more than one GVHD prophylactic agent (for example, a single GVHD prophylactic agent) develop ≥grade 4 aGVHD. In some embodiments, less than about 5% of subjects that are administered a composition of the disclosure and no more than one GVHD prophylactic agent (for example, a single GVHD prophylactic agent) develop ≥grade 4 aGVHD. In some embodiments, less than about 3% of subjects that are administered a composition of the disclosure and no more than one GVHD prophylactic agent (for example, a single GVHD prophylactic agent) develop ≥grade 4 aGVHD. In some embodiments, less than about 2% of subjects that are administered a composition of the disclosure and no more than one GVHD prophylactic agent (for example, a single GVHD prophylactic agent) develop ≥grade 4 aGVHD. In some embodiments, less than about 1% of subjects that are administered a composition of the disclosure and no more than one GVHD prophylactic agent (for example, a single GVHD prophylactic agent) develop ≥grade 4 aGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) may exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 2 aGVHD. In some embodiments, less than about 15% of subjects that are administered a composition of the disclosure and a low dose of a GVHD prophylactic agent (for example, a single GVHD prophylactic agent at a low dose) develop ≥grade 2 aGVHD. In some embodiments, less than about 10% of subjects that are administered a composition of the disclosure and a low dose of a GVHD prophylactic agent (for example, a single GVHD prophylactic agent at a low dose) develop ≥grade 2 aGVHD. In some embodiments, less than about 8% of subjects that are administered a composition of the disclosure and a low dose of a GVHD prophylactic agent (for example, a single GVHD prophylactic agent at a low dose) develop ≥grade 2 aGVHD. In some embodiments, less than about 7% of subjects that are administered a composition of the disclosure and a low dose of a GVHD prophylactic agent (for example, a single GVHD prophylactic agent at a low dose) develop ≥grade 2 aGVHD.

Subjects administered a composition of the disclosure (e.g., a cell population as described herein) and a low dose of a GVHD prophylactic agent (for example, a single GVHD prophylactic agent at a low dose) may exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 3 aGVHD. In some embodiments, less than about 15% of subjects that are administered a composition of the disclosure and a low dose of a GVHD prophylactic agent (for example, a single GVHD prophylactic agent at a low dose) develop ≥grade 3 aGVHD. In some embodiments, less than about 10% of subjects that are administered a composition of the disclosure and a low dose of a GVHD prophylactic agent (for example, a single GVHD prophylactic agent at a low dose) develop ≥grade 3 aGVHD. In some embodiments, less than about 5% of subjects that are administered a composition of the disclosure and a low dose of a GVHD prophylactic agent (for example, a single GVHD prophylactic agent at a low dose) develop ≥grade 3 aGVHD. In some embodiments, less than about 3% of subjects that are administered a composition of the disclosure and a low dose of a GVHD prophylactic agent (for example, a single GVHD prophylactic agent at a low dose) develop ≥grade 3 aGVHD. In some embodiments, less than about 2% of subjects that are administered a composition of the disclosure and a low dose of a GVHD prophylactic agent (for example, a single GVHD prophylactic agent at a low dose) develop ≥grade 3 aGVHD. In some embodiments, less than about 1% of subjects that are administered a composition of the disclosure and a low dose of a GVHD prophylactic agent (for example, a single GVHD prophylactic agent at a low dose) develop ≥grade 3 aGVHD.

Subjects administered a composition of the disclosure (e.g., a cell population as described herein) and a low dose of a GVHD prophylactic agent (for example, a single GVHD prophylactic agent at a low dose) may exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and a low dose of a GVHD prophylactic agent (for example, a single GVHD prophylactic agent at a low dose) develop ≥grade 4 aGVHD. In some embodiments, less than about 5% of subjects that are administered a composition of the disclosure and a low dose of a GVHD prophylactic agent (for example, a single GVHD prophylactic agent at a low dose) develop ≥grade 4 aGVHD. In some embodiments, less than about 3% of subjects that are administered a composition of the disclosure and a low dose of a GVHD prophylactic agent (for example, a single GVHD prophylactic agent at a low dose) develop ≥grade 4 aGVHD. In some embodiments, less than about 2% of subjects that are administered a composition of the disclosure and a low dose of a GVHD prophylactic agent (for example, a single GVHD prophylactic agent at a low dose) develop ≥grade 4 aGVHD. In some embodiments, less than about 1% of subjects that are administered a composition of the disclosure and a low dose of a GVHD prophylactic agent (for example, a single GVHD prophylactic agent at a low dose) develop ≥grade 4 aGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Chronic GVHD Incidence

Subjects administered a composition of the disclosure (e.g., a cell population as described herein) may exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) may exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 2 cGVHD. In some embodiments, less than about 40% of subjects that are administered a composition of the disclosure develop ≥grade 2 cGVHD. In some embodiments, less than about 30% of subjects that are administered a composition of the disclosure develop ≥grade 2 cGVHD. In some embodiments, less than about 20% of subjects that are administered a composition of the disclosure develop ≥grade 2 cGVHD. In some embodiments, less than about 10% of subjects that are administered a composition of the disclosure develop ≥grade 2 cGVHD. In some embodiments, less than about 5% of subjects that are administered a composition of the disclosure develop ≥grade 2 cGVHD.

Subjects administered a composition of the disclosure (e.g., a cell population as described herein) may exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 3 cGVHD. In some embodiments, less than about 40% of subjects that are administered a composition of the disclosure develop ≥grade 3 cGVHD. In some embodiments, less than about 30% of subjects that are administered a composition of the disclosure develop ≥grade 3 cGVHD. In some embodiments, less than about 20% of subjects that are administered a composition of the disclosure develop ≥grade 3 cGVHD. In some embodiments, less than about 15% of subjects that are administered a composition of the disclosure develop ≥grade 3 cGVHD. In some embodiments, less than about 10% of subjects that are administered a composition of the disclosure develop ≥grade 3 cGVHD. In some embodiments, less than about 5% of subjects that are administered a composition of the disclosure develop ≥grade 3 cGVHD. In some embodiments, less than about 3% of subjects that are administered a composition of the disclosure develop ≥grade 3 cGVHD. In some embodiments, less than about 2% of subjects that are administered a composition of the disclosure develop ≥grade 3 cGVHD. In some embodiments, less than about 1% of subjects that are administered a composition of the disclosure develop ≥grade 3 cGVHD.

Subjects administered a composition of the disclosure (e.g., a cell population as described herein) may exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure develop ≥grade 4 cGVHD. In some embodiments, less than about 30% of subjects that are administered a composition of the disclosure develop ≥grade 4 cGVHD. In some embodiments, less than about 20% of subjects that are administered a composition of the disclosure develop ≥grade 4 cGVHD. In some embodiments, less than about 10% of subjects that are administered a composition of the disclosure develop ≥grade 4 cGVHD. In some embodiments, less than about 5% of subjects that are administered a composition of the disclosure develop ≥grade 4 cGVHD. In some embodiments, less than about 3% of subjects that are administered a composition of the disclosure develop ≥grade 4 cGVHD. In some embodiments, less than about 2% of subjects that are administered a composition of the disclosure develop ≥grade 4 cGVHD. In some embodiments, less than about 1% of subjects that are administered a composition of the disclosure develop ≥grade 4 cGVHD.

Subjects administered a composition of the disclosure (e.g., a cell population as described herein) may exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop mild to severe cGVHD, for example, within 365 days post-transplant, two years post-transplant, or within another suitable amount of time post-transplant as disclosed herein.

Subjects administered a composition of the disclosure (e.g., a cell population as described herein) may exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure develop moderate to severe cGVHD. In some embodiments, less than about 40% of subjects that are administered a composition of the disclosure develop moderate to severe cGVHD. In some embodiments, less than about 30% of subjects that are administered a composition of the disclosure develop moderate to severe cGVHD. In some embodiments, less than about 20% of subjects that are administered a composition of the disclosure develop moderate to severe cGVHD. In some embodiments, less than about 10% of subjects that are administered a composition of the disclosure develop moderate to severe cGVHD. In some embodiments, less than about 5% of subjects that are administered a composition of the disclosure develop moderate to severe cGVHD.

Subjects administered a composition of the disclosure (e.g., a cell population as described herein) may exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop severe cGVHD, for example, within 365 days post-transplant, two years post-transplant, or within another suitable amount of time post-transplant as disclosed herein.

In some embodiments, less than about 50% of subjects that are administered a composition of the disclosure develop severe cGVHD. In some embodiments, less than about 40% of subjects that are administered a composition of the disclosure develop severe cGVHD. In some embodiments, less than about 30% of subjects that are administered a composition of the disclosure develop severe cGVHD. In some embodiments, less than about 20% of subjects that are administered a composition of the disclosure develop severe cGVHD. In some embodiments, less than about 15% of subjects that are administered a composition of the disclosure develop severe cGVHD. In some embodiments, less than about 10% of subjects that are administered a composition of the disclosure develop severe cGVHD. In some embodiments, less than about 5% of subjects that are administered a composition of the disclosure develop severe cGVHD. In some embodiments, less than about 3% of subjects that are administered a composition of the disclosure develop severe cGVHD. In some embodiments, less than about 2% of subjects that are administered a composition of the disclosure develop severe cGVHD. In some embodiments, less than about 1% of subjects that are administered a composition of the disclosure develop severe cGVHD.

The incidence of cGVHD can be assessed after a suitable amount of time elapses post-transplant, for example, about 150 days, about 200 days, about 365 days, about 1.5 years, about 2 years, about 2.5 years, about 3 years, about 3.5 years, about 4 years, about 4.5 years, or about 5 years post-transplant.

The incidence of cGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population as described herein) have a decreased incidence, severity, timing, or any combination thereof of Grade I to Grade IV chronic GVHD (cGVHD) or severe cGVHD, relative to a corresponding human subject that has been administered a standard myeloablative allogeneic hematopoietic stem cell transplant (alloHSCT). In some embodiments, the subject is free of Grade I to Grade IV cGVHD or severe cGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, subjects administered a composition of the disclosure (e.g., a cell population as described herein) have increased cGVHD survival, relative to a corresponding human subject that has been administered a standard myeloablative allogeneic hematopoietic stem cell transplant (alloHSCT). In some embodiments, the subject experiences cGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

### No GVHD Prophylaxis

Subjects administered a composition of the disclosure (e.g., a cell population as described herein) in the absence of GVHD prophylactic agents may exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents may exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 2 cGVHD. In some embodiments, less than about 40% of subjects that are administered a composition of the disclosure in the absence of GVHD prophylactic agents develop ≥grade 2 cGVHD. In some embodiments, less than about 30% of subjects that are administered a composition of the disclosure in the absence of GVHD prophylactic agents develop ≥grade 2 cGVHD. In some embodiments, less than about 25% of subjects that are administered a composition of the disclosure in the absence of GVHD prophylactic agents develop ≥grade 2 cGVHD. In some embodiments, less than about 20% of subjects that are administered a composition of the disclosure in the absence of GVHD prophylactic agents develop ≥grade 2 cGVHD. In some embodiments, less than about 10% of subjects that are administered a composition of the disclosure in the absence of GVHD prophylactic agents develop ≥grade 2 cGVHD. In some embodiments, less than about 5% of subjects that are administered a composition of the disclosure in the absence of GVHD prophylactic agents develop ≥grade 2 cGVHD.

Subjects administered a composition of the disclosure (e.g., a cell population as described herein) in the absence of GVHD prophylactic agents may exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 3 cGVHD. In some embodiments, less than about 40% of subjects that are administered a composition of the disclosure in the absence of GVHD prophylactic agents develop ≥grade 3 cGVHD. In some embodiments, less than about 30% of subjects that are administered a composition of the disclosure in the absence of GVHD prophylactic agents develop ≥grade 3 cGVHD. In some embodiments, less than about 20% of subjects that are administered a composition of the disclosure in the absence of GVHD prophylactic agents develop ≥grade 3 cGVHD. In some embodiments, less than about 10% of subjects that are administered a composition of the disclosure in the absence of GVHD prophylactic agents develop ≥grade 3 cGVHD. In some embodiments, less than about 5% of subjects that are administered a composition of the disclosure in the absence of GVHD prophylactic agents develop ≥grade 3 cGVHD. In some embodiments, less than about 3% of subjects that are administered a composition of the disclosure in the absence of GVHD prophylactic agents develop ≥grade 3 cGVHD. In some embodiments, less than about 2% of subjects that are administered a composition of the disclosure in the absence of GVHD prophylactic agents develop ≥grade 3 cGVHD. In some embodiments, less than about 1% of subjects that are administered a composition of the disclosure in the absence of GVHD prophylactic agents develop ≥grade 3 cGVHD.

Subjects administered a composition of the disclosure (e.g., a cell population as described herein) in the absence of GVHD prophylactic agents may exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, or less than about 50% of subjects that are administered a composition of the disclosure in the absence of GVHD prophylactic agents develop ≥grade 4 cGVHD, for example, within 365 days post-transplant, two years post-transplant, or within another suitable amount of time post-transplant as disclosed herein.

In some embodiments, less than about 30% of subjects that are administered a composition of the disclosure in the absence of GVHD prophylactic agents develop ≥grade 4 cGVHD. In some embodiments, less than about 20% of subjects that are administered a composition of the disclosure in the absence of GVHD prophylactic agents develop ≥grade 4 cGVHD. In some embodiments, less than about 15% of subjects that are administered a composition of the disclosure in the absence of GVHD prophylactic agents develop ≥grade 4 cGVHD. In some embodiments, less than about 10% of subjects that are administered a composition of the disclosure in the absence of GVHD prophylactic agents develop ≥grade 4 cGVHD. In some embodiments, less than about 5% of subjects that are administered a composition of the disclosure in the absence of GVHD prophylactic agents develop ≥grade 4 cGVHD. In some embodiments, less than about 3% of subjects that are administered a composition of the disclosure in the absence of GVHD prophylactic agents develop ≥grade 4 cGVHD. In some embodiments, less than about 2% of subjects that are administered a composition of the disclosure in the absence of GVHD prophylactic agents develop ≥grade 4 cGVHD. In some embodiments, less than about 1% of subjects that are administered a composition of the disclosure in the absence of GVHD prophylactic agents develop ≥grade 4 cGVHD.

Subjects administered a composition of the disclosure (e.g., a cell population as described herein) in the absence of GVHD prophylactic agents may exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop mild to severe cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents may exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop moderate to severe cGVHD. In some embodiments, less than about 40% of subjects that are administered a composition of the disclosure in the absence of GVHD prophylactic agents develop moderate to severe cGVHD. In some embodiments, less than about 30% of subjects that are administered a composition of the disclosure in the absence of GVHD prophylactic agents develop moderate to severe cGVHD. In some embodiments, less than about 25% of subjects that are administered a composition of the disclosure in the absence of GVHD prophylactic agents develop moderate to severe cGVHD. In some embodiments, less than about 20% of subjects that are administered a composition of the disclosure in the absence of GVHD prophylactic agents develop moderate to severe cGVHD. In some embodiments, less than about 10% of subjects that are administered a composition of the disclosure in the absence of GVHD prophylactic agents develop moderate to severe cGVHD. In some embodiments, less than about 5% of subjects that are administered a composition of the disclosure in the absence of GVHD prophylactic agents develop moderate to severe cGVHD.

Subjects administered a composition of the disclosure (e.g., a cell population as described herein) in the absence of GVHD prophylactic agents may exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop severe cGVHD. In some embodiments, less than about 40% of subjects that are administered a composition of the disclosure in the absence of GVHD prophylactic agents develop severe cGVHD. In some embodiments, less than about 30% of subjects that are administered a composition of the disclosure in the absence of GVHD prophylactic agents develop severe cGVHD. In some embodiments, less than about 20% of subjects that are administered a composition of the disclosure in the absence of GVHD prophylactic agents develop severe cGVHD. In some embodiments, less than about 10% of subjects that are administered a composition of the disclosure in the absence of GVHD prophylactic agents develop severe cGVHD. In some embodiments, less than about 5% of subjects that are administered a composition of the disclosure in the absence of GVHD prophylactic agents develop severe cGVHD. In some embodiments, less than about 3% of subjects that are administered a composition of the disclosure in the absence of GVHD prophylactic agents develop severe cGVHD. In some embodiments, less than about 2% of subjects that are administered a composition of the disclosure in the absence of GVHD prophylactic agents develop severe cGVHD. In some embodiments, less than about 1% of subjects that are administered a composition of the disclosure in the absence of GVHD prophylactic agents develop severe cGVHD.

The absence of GVHD prophylactic agents can refer to cases where no GVHD prophylactic agents are administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant.

### Single Agent GVHD Prophylaxis

Subjects administered a composition of the disclosure (e.g., a cell population as described herein) and no more than one GVHD prophylactic agent (for example, a single GVHD prophylactic agent) may exhibit a low incidence of ≥grade 1 cGVHD, for example, a lower incidence of ≥grade 1 cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) may exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 2 cGVHD. In some embodiments, less than about 40% of subjects that are administered a composition of the disclosure and no more than one GVHD prophylactic agent (for example, a single GVHD prophylactic agent) develop ≥grade 2 cGVHD. In some embodiments, less than about 30% of subjects that are administered a composition of the disclosure and no more than one GVHD prophylactic agent (for example, a single GVHD prophylactic agent) develop ≥grade 2 cGVHD. In some embodiments, less than about 20% of subjects that are administered a composition of the disclosure and no more than one GVHD prophylactic agent (for example, a single GVHD prophylactic agent) develop ≥grade 2 cGVHD. In some embodiments, less than about 10% of subjects that are administered a composition of the disclosure and no more than one GVHD prophylactic agent (for example, a single GVHD prophylactic agent) develop ≥grade 2 cGVHD. In some embodiments, less than about 5% of subjects that are administered a composition of the disclosure and no more than one GVHD prophylactic agent (for example, a single GVHD prophylactic agent) develop ≥grade 2 cGVHD.

Subjects administered a composition of the disclosure (e.g., a cell population as described herein) and no more than one GVHD prophylactic agent (for example, a single GVHD prophylactic agent) may exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 3 cGVHD. In some embodiments, less than about 40% of subjects that are administered a composition of the disclosure and no more than one GVHD prophylactic agent (for example, a single GVHD prophylactic agent) develop ≥grade 3 cGVHD. In some embodiments, less than about 30% of subjects that are administered a composition of the disclosure and no more than one GVHD prophylactic agent (for example, a single GVHD prophylactic agent) develop ≥grade 3 cGVHD. In some embodiments, less than about 20% of subjects that are administered a composition of the disclosure and no more than one GVHD prophylactic agent (for example, a single GVHD prophylactic agent) develop ≥grade 3 cGVHD. In some embodiments, less than about 15% of subjects that are administered a composition of the disclosure and no more than one GVHD prophylactic agent (for example, a single GVHD prophylactic agent) develop ≥grade 3 cGVHD. In some embodiments, less than about 10% of subjects that are administered a composition of the disclosure and no more than one GVHD prophylactic agent (for example, a single GVHD prophylactic agent) develop ≥grade 3 cGVHD. In some embodiments, less than about 5% of subjects that are administered a composition of the disclosure and no more than one GVHD prophylactic agent (for example, a single GVHD prophylactic agent) develop ≥grade 3 cGVHD. In some embodiments, less than about 3% of subjects that are administered a composition of the disclosure and no more than one GVHD prophylactic agent (for example, a single GVHD prophylactic agent) develop ≥grade 3 cGVHD. In some embodiments, less than about 2% of subjects that are administered a composition of the disclosure and no more than one GVHD prophylactic agent (for example, a single GVHD prophylactic agent) develop ≥grade 3 cGVHD. In some embodiments, less than about 1% of subjects that are administered a composition of the disclosure and no more than one GVHD prophylactic agent (for example, a single GVHD prophylactic agent) develop ≥grade 3 cGVHD.

Subjects administered a composition of the disclosure (e.g., a cell population as described herein) and no more than one GVHD prophylactic agent (for example, a single GVHD prophylactic agent) may exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and no more than one GVHD prophylactic agent (for example, a single GVHD prophylactic agent) develop ≥grade 4 cGVHD. In some embodiments, less than about 30% of subjects that are administered a composition of the disclosure and no more than one GVHD prophylactic agent (for example, a single GVHD prophylactic agent) develop ≥grade 4 cGVHD. In some embodiments, less than about 20% of subjects that are administered a composition of the disclosure and no more than one GVHD prophylactic agent (for example, a single GVHD prophylactic agent) develop ≥grade 4 cGVHD. In some embodiments, less than about 10% of subjects that are administered a composition of the disclosure and no more than one GVHD prophylactic agent (for example, a single GVHD prophylactic agent) develop ≥grade 4 cGVHD. In some embodiments, less than about 5% of subjects that are administered a composition of the disclosure and no more than one GVHD prophylactic agent (for example, a single GVHD prophylactic agent) develop ≥grade 4 cGVHD. In some embodiments, less than about 3% of subjects that are administered a composition of the disclosure and no more than one GVHD prophylactic agent (for example, a single GVHD prophylactic agent) develop ≥grade 4 cGVHD. In some embodiments, less than about 2% of subjects that are administered a composition of the disclosure and no more than one GVHD prophylactic agent (for example, a single GVHD prophylactic agent) develop ≥grade 4 cGVHD. In some embodiments, less than about 1% of subjects that are administered a composition of the disclosure and no more than one GVHD prophylactic agent (for example, a single GVHD prophylactic agent) develop ≥grade 4 cGVHD.

Subjects administered a composition of the disclosure (e.g., a cell population as described herein) and no more than one GVHD prophylactic agent (for example, a single GVHD prophylactic agent) may exhibit a low incidence of mild to severe cGVHD, for example, a lower incidence of mild to severe cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) may exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop moderate to severe cGVHD. In some embodiments, less than about 40% of subjects that are administered a composition of the disclosure and no more than one GVHD prophylactic agent (for example, a single GVHD prophylactic agent) develop moderate to severe cGVHD. In some embodiments, less than about 30% of subjects that are administered a composition of the disclosure and no more than one GVHD prophylactic agent (for example, a single GVHD prophylactic agent) develop moderate to severe cGVHD. In some embodiments, less than about 20% of subjects that are administered a composition of the disclosure and no more than one GVHD prophylactic agent (for example, a single GVHD prophylactic agent) develop moderate to severe cGVHD. In some embodiments, less than about 10% of subjects that are administered a composition of the disclosure and no more than one GVHD prophylactic agent (for example, a single GVHD prophylactic agent) develop moderate to severe cGVHD. In some embodiments, less than about 5% of subjects that are administered a composition of the disclosure and no more than one GVHD prophylactic agent (for example, a single GVHD prophylactic agent) develop moderate to severe cGVHD.

Subjects administered a composition of the disclosure (e.g., a cell population as described herein) and no more than one GVHD prophylactic agent (for example, a single GVHD prophylactic agent) may exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop severe cGVHD. In some embodiments, less than about 40% of subjects that are administered a composition of the disclosure and no more than one GVHD prophylactic agent (for example, a single GVHD prophylactic agent) develop severe cGVHD. In some embodiments, less than about 30% of subjects that are administered a composition of the disclosure and no more than one GVHD prophylactic agent (for example, a single GVHD prophylactic agent) develop severe cGVHD. In some embodiments, less than about 20% of subjects that are administered a composition of the disclosure and no more than one GVHD prophylactic agent (for example, a single GVHD prophylactic agent) develop severe cGVHD. In some embodiments, less than about 15% of subjects that are administered a composition of the disclosure and no more than one GVHD prophylactic agent (for example, a single GVHD prophylactic agent) develop severe cGVHD. In some embodiments, less than about 10% of subjects that are administered a composition of the disclosure and no more than one GVHD prophylactic agent (for example, a single GVHD prophylactic agent) develop severe cGVHD. In some embodiments, less than about 5% of subjects that are administered a composition of the disclosure and no more than one GVHD prophylactic agent (for example, a single GVHD prophylactic agent) develop severe cGVHD. In some embodiments, less than about 3% of subjects that are administered a composition of the disclosure and no more than one GVHD prophylactic agent (for example, a single GVHD prophylactic agent) develop severe cGVHD. In some embodiments, less than about 2% of subjects that are administered a composition of the disclosure and no more than one GVHD prophylactic agent (for example, a single GVHD prophylactic agent) develop severe cGVHD. In some embodiments, less than about 1% of subjects that are administered a composition of the disclosure and no more than one GVHD prophylactic agent (for example, a single GVHD prophylactic agent) develop severe cGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) may exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) may exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 2 cGVHD. In some embodiments, less than about 40% of subjects that are administered a composition of the disclosure and a low dose of a GVHD prophylactic agent (for example, a single GVHD prophylactic agent at a low dose) develop ≥grade 2 cGVHD. In some embodiments, less than about 30% of subjects that are administered a composition of the disclosure and a low dose of a GVHD prophylactic agent (for example, a single GVHD prophylactic agent at a low dose) develop ≥grade 2 cGVHD. In some embodiments, less than about 20% of subjects that are administered a composition of the disclosure and a low dose of a GVHD prophylactic agent (for example, a single GVHD prophylactic agent at a low dose) develop ≥grade 2 cGVHD. In some embodiments, less than about 15% of subjects that are administered a composition of the disclosure and a low dose of a GVHD prophylactic agent (for example, a single GVHD prophylactic agent at a low dose) develop ≥grade 2 cGVHD. In some embodiments, less than about 10% of subjects that are administered a composition of the disclosure and a low dose of a GVHD prophylactic agent (for example, a single GVHD prophylactic agent at a low dose) develop ≥grade 2 cGVHD. In some embodiments, less than about 5% of subjects that are administered a composition of the disclosure and a low dose of a GVHD prophylactic agent (for example, a single GVHD prophylactic agent at a low dose) develop ≥grade 2 cGVHD.

Subjects administered a composition of the disclosure (e.g., a cell population as described herein) and a low dose of a GVHD prophylactic agent (for example, a single GVHD prophylactic agent at a low dose) may exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 3 cGVHD. In some embodiments, less than about 40% of subjects that are administered a composition of the disclosure and a low dose of a GVHD prophylactic agent (for example, a single GVHD prophylactic agent at a low dose) develop ≥grade 3 cGVHD. In some embodiments, less than about 30% of subjects that are administered a composition of the disclosure and a low dose of a GVHD prophylactic agent (for example, a single GVHD prophylactic agent at a low dose) develop ≥grade 3 cGVHD. In some embodiments, less than about 20% of subjects that are administered a composition of the disclosure and a low dose of a GVHD prophylactic agent (for example, a single GVHD prophylactic agent at a low dose) develop ≥grade 3 cGVHD. In some embodiments, less than about 15% of subjects that are administered a composition of the disclosure and a low dose of a GVHD prophylactic agent (for example, a single GVHD prophylactic agent at a low dose) develop ≥grade 3 cGVHD. In some embodiments, less than about 10% of subjects that are administered a composition of the disclosure and a low dose of a GVHD prophylactic agent (for example, a single GVHD prophylactic agent at a low dose) develop ≥grade 3 cGVHD. In some embodiments, less than about 5% of subjects that are administered a composition of the disclosure and a low dose of a GVHD prophylactic agent (for example, a single GVHD prophylactic agent at a low dose) develop ≥grade 3 cGVHD. In some embodiments, less than about 3% of subjects that are administered a composition of the disclosure and a low dose of a GVHD prophylactic agent (for example, a single GVHD prophylactic agent at a low dose) develop ≥grade 3 cGVHD. In some embodiments, less than about 2% of subjects that are administered a composition of the disclosure and a low dose of a GVHD prophylactic agent (for example, a single GVHD prophylactic agent at a low dose) develop ≥grade 3 cGVHD. In some embodiments, less than about 1% of subjects that are administered a composition of the disclosure and a low dose of a GVHD prophylactic agent (for example, a single GVHD prophylactic agent at a low dose) develop ≥grade 3 cGVHD.

Subjects administered a composition of the disclosure (e.g., a cell population as described herein) and a low dose of a GVHD prophylactic agent (for example, a single GVHD prophylactic agent at a low dose) may exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and a low dose of a GVHD prophylactic agent (for example, a single GVHD prophylactic agent at a low dose) develop ≥grade 4 cGVHD. In some embodiments, less than about 30% of subjects that are administered a composition of the disclosure and a low dose of a GVHD prophylactic agent (for example, a single GVHD prophylactic agent at a low dose) develop ≥grade 4 cGVHD. In some embodiments, less than about 20% of subjects that are administered a composition of the disclosure and a low dose of a GVHD prophylactic agent (for example, a single GVHD prophylactic agent at a low dose) develop ≥grade 4 cGVHD. In some embodiments, less than about 10% of subjects that are administered a composition of the disclosure and a low dose of a GVHD prophylactic agent (for example, a single GVHD prophylactic agent at a low dose) develop ≥grade 4 cGVHD. In some embodiments, less than about 5% of subjects that are administered a composition of the disclosure and a low dose of a GVHD prophylactic agent (for example, a single GVHD prophylactic agent at a low dose) develop ≥grade 4 cGVHD. In some embodiments, less than about 3% of subjects that are administered a composition of the disclosure and a low dose of a GVHD prophylactic agent (for example, a single GVHD prophylactic agent at a low dose) develop ≥grade 4 cGVHD. In some embodiments, less than about 2% of subjects that are administered a composition of the disclosure and a low dose of a GVHD prophylactic agent (for example, a single GVHD prophylactic agent at a low dose) develop ≥grade 4 cGVHD. In some embodiments, less than about 1% of subjects that are administered a composition of the disclosure and a low dose of a GVHD prophylactic agent (for example, a single GVHD prophylactic agent at a low dose) develop ≥grade 4 cGVHD.

Subjects administered a composition of the disclosure (e.g., a cell population as described herein) and a low dose of a GVHD prophylactic agent (for example, a single GVHD prophylactic agent at a low dose) may exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) may exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop moderate to severe cGVHD. In some embodiments, less than about 40% of subjects that are administered a composition of the disclosure and a low dose of a GVHD prophylactic agent (for example, a single GVHD prophylactic agent at a low dose) develop moderate to severe cGVHD. In some embodiments, less than about 30% of subjects that are administered a composition of the disclosure and a low dose of a GVHD prophylactic agent (for example, a single GVHD prophylactic agent at a low dose) develop moderate to severe cGVHD. In some embodiments, less than about 20% of subjects that are administered a composition of the disclosure and a low dose of a GVHD prophylactic agent (for example, a single GVHD prophylactic agent at a low dose) develop moderate to severe cGVHD. In some embodiments, less than about 15% of subjects that are administered a composition of the disclosure and a low dose of a GVHD prophylactic agent (for example, a single GVHD prophylactic agent at a low dose) develop moderate to severe cGVHD. In some embodiments, less than about 10% of subjects that are administered a composition of the disclosure and a low dose of a GVHD prophylactic agent (for example, a single GVHD prophylactic agent at a low dose) develop moderate to severe cGVHD. In some embodiments, less than about 5% of subjects that are administered a composition of the disclosure and a low dose of a GVHD prophylactic agent (for example, a single GVHD prophylactic agent at a low dose) develop moderate to severe cGVHD.

Subjects administered a composition of the disclosure (e.g., a cell population as described herein) and a low dose of a GVHD prophylactic agent (for example, a single GVHD prophylactic agent at a low dose) may exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop severe cGVHD. In some embodiments, less than about 40% of subjects that are administered a composition of the disclosure and a low dose of a GVHD prophylactic agent (for example, a single GVHD prophylactic agent at a low dose) develop severe cGVHD. In some embodiments, less than about 30% of subjects that are administered a composition of the disclosure and a low dose of a GVHD prophylactic agent (for example, a single GVHD prophylactic agent at a low dose) develop severe cGVHD. In some embodiments, less than about 20% of subjects that are administered a composition of the disclosure and a low dose of a GVHD prophylactic agent (for example, a single GVHD prophylactic agent at a low dose) develop severe cGVHD. In some embodiments, less than about 15% of subjects that are administered a composition of the disclosure and a low dose of a GVHD prophylactic agent (for example, a single GVHD prophylactic agent at a low dose) develop severe cGVHD. In some embodiments, less than about 10% of subjects that are administered a composition of the disclosure and a low dose of a GVHD prophylactic agent (for example, a single GVHD prophylactic agent at a low dose) develop severe cGVHD. In some embodiments, less than about 5% of subjects that are administered a composition of the disclosure and a low dose of a GVHD prophylactic agent (for example, a single GVHD prophylactic agent at a low dose) develop severe cGVHD. In some embodiments, less than about 3% of subjects that are administered a composition of the disclosure and a low dose of a GVHD prophylactic agent (for example, a single GVHD prophylactic agent at a low dose) develop severe cGVHD. In some embodiments, less than about 2% of subjects that are administered a composition of the disclosure and a low dose of a GVHD prophylactic agent (for example, a single GVHD prophylactic agent at a low dose) develop severe cGVHD. In some embodiments, less than about 1% of subjects that are administered a composition of the disclosure and a low dose of a GVHD prophylactic agent (for example, a single GVHD prophylactic agent at a low dose) develop severe cGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Organ-specific GVHD Stage Incidence

Subjects administered a composition of the disclosure (e.g., a cell population as described herein) may exhibit a low incidence of ≥stage 1 GVHD signs for the skin, liver, gut, or a combination thereof, for example, a lower incidence compared to subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure exhibit ≥stage 1 GVHD signs for the skin, liver, gut, or a combination thereof.

Subjects administered a composition of the disclosure (e.g., a cell population as described herein) may exhibit a low incidence of ≥stage 2 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 1 GVHD signs for the skin, liver, gut, or a combination thereof.

Subjects administered a composition of the disclosure (e.g., a cell population as described herein) may exhibit a low incidence of ≥stage 3 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 3 GVHD signs for the skin, liver, gut, or a combination thereof.

Subjects administered a composition of the disclosure (e.g., a cell population as described herein) may exhibit a low incidence of ≥stage 4 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 4 GVHD signs for the skin, liver, gut, or a combination thereof.

The incidence of the organ-specific GVHD signs can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days, about 150 days, about 200 days, about 365 days, about 1.5 years, about 2 years, about 2.5 years, about 3 years, about 3.5 years, about 4 years, about 4.5 years, or about 5 years post-transplant.

The incidence of the organ-specific GVHD signs can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### Other Clinical Outcomes

### Survival, Hospitalization, and Relapse

In some embodiments, patient treated or provided with compositions, cellular therapy products, pharmaceutical treatments, cell populations, solutions, formulations, kits, and/or methods has a reduced risk of at least one of malignancy relapse, infection or renal failure.

Subjects treated or provided with compositions, cellular therapy products, pharmaceutical treatments, cell populations, solutions, formulations, kits, and/or methods of the disclosure (e.g., a cell population comprising a population of cells described herein) may exhibit a high overall survival rate, for example, a higher overall survival rate compared to subjects that are administered an alternate composition. In some embodiments, at least about 40%, at least about 50%, at least about 55%, at least about 60%, at least about 65%, at least about 70%, at least about 75%, at least about 80%, at least about 85%, at least about 90%, or at least about 95% of subjects that are administered a composition of the disclosure are alive after about 1 year, about 1.5 years, about 2 years, about 2.5 years, about 3 years, about 3.5 years, about 4 years, about 4.5 years, about 5 years, about 7 years, about 10 years, about 15 years, about 20 years, about 30 years post-transplant.

Subjects treated or provided with compositions, cellular therapy products, pharmaceutical treatments, cell populations, solutions, formulations, kits, and/or methods of the disclosure (e.g., a cell population comprising a population of cells described herein) may exhibit a low treatment-associated mortality rate, for example, a lower treatment-associated mortality rate compared to subjects that are administered an alternate composition. In some embodiments, a treatment-associated mortality rate of a population of subjects administered a composition of the disclosure is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 25%, less than about 30%, less than about 35%, less than about 40%, or less than about 50% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments a treatment-associated mortality rate of a population of subjects administered a composition of the disclosure is less than about 5% when evaluated at 1-year post-transplant. In some embodiments a treatment-associated mortality rate of a population of subjects administered a composition of the disclosure is less than about 1% when evaluated at 1-year post-transplant.

Subjects treated or provided with compositions, cellular therapy products, pharmaceutical treatments, cell populations, solutions, formulations, kits, and/or methods of the disclosure (e.g., a cell population comprising a population of cells described herein) may exhibit GVHD free and relapse free survival (GRFS) rate, for example, a higher GRFS rate compared to subjects that are administered an alternate composition. In some embodiments, GRFS can refer to survival without relapse or Grade ≥3 aGVHD or extensive (e.g., severe) cGVHD. In some embodiments, GRFS can refer to survival without relapse or Grade ≥2 aGVHD or extensive (e.g., moderate to severe) cGVHD. In some embodiments, GRFS can refer to survival with no GVHD symptoms. In some embodiments, a GRFS rate of a population of subjects administered a composition of the disclosure is at least about 20%, at least about 30%, at least about 40%, at least about 50%, at least about 55%, at least about 60%, at least about 65%, at least about 70%, at least about 75%, at least about 80%, at least about 85%, at least about 90%, or at least about 95% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments, a GRFS rate of a population of subjects administered a composition of the disclosure is at least about 60% when evaluated at 1-year post-transplant. In some embodiments, a GRFS rate of a population of subjects administered a composition of the disclosure is at least about 75% when evaluated at 1-year post-transplant. In some embodiments, a GRFS rate of a population of subjects administered a composition of the disclosure is more than 75% when evaluated at 3 years post-transplant. In some embodiments, a GRFS rate of a population of subjects administered a composition of the disclosure is more than 75% when evaluated at 5 years post-transplant. In some embodiments, a GRFS rate of a population of subjects administered a composition of the disclosure is more than 75% when evaluated at 7 years post-transplant. In some embodiments, a GRFS rate of a population of subjects administered a composition of the disclosure is more than 75% when evaluated at 10 years post-transplant.

Subjects treated or provided with compositions, cellular therapy products, pharmaceutical treatments, cell populations, solutions, formulations, kits, and/or methods of the disclosure (e.g., a cell population comprising a population of cells described herein) may exhibit a short time to discharge from hospital, for example, a shorter time to discharge from hospital compared to subjects that are administered an alternate composition. In some embodiments, the average time to discharge after day 0 of a transplantation regimen is less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, or less than about 8 days. In some embodiments, the average time to discharge after day 0 of a transplantation regimen is less than about 17 days. In some embodiments, the average time to discharge after day 0 of a transplantation regimen is less than about 18 days. In embodiments, the human subject treated or provided with compositions, pharmaceutical treatments, cell populations, solutions, formulations, kits, and/or methods as disclosed herein have no relapse of their malignancy about one year after being administered the pharmaceutical dosing regimen.

Subjects treated or provided with compositions, cellular therapy products, pharmaceutical treatments, cell populations, solutions, formulations, kits, and/or methods of the disclosure (e.g., a cell population comprising a population of cells described herein) may exhibit a low relapse rate, for example, a lower relapse rate compared to subjects that are administered an alternate composition. In some embodiments, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that are administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 35% of subjects that are administered a composition of the disclosure relapse within 1-year post-transplant. In some embodiments, less than about 25% of subjects that are administered a composition of the disclosure relapse within 1-year post-transplant.

Subjects that do not have active disease when treated or provided with compositions, cellular therapy products, pharmaceutical treatments, cell populations, solutions, formulations, kits, and/or methods of the disclosure (e.g., a cell population as described herein) may exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that do not have active disease when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient treated or provided with compositions, cellular therapy products, pharmaceutical treatments, cell populations, solutions, formulations, kits, and/or methods has no relapse of their malignancy about one year after being administered the cell populations.

Subjects that are in complete remission when administered a composition of the disclosure (e.g., a cell population as described herein) may exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, or less than about 40% of subjects that are in complete remission when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient has no GHVD or relapse of their malignancy one year after being administered the cell populations.

In some embodiments, subjects administered a composition of the disclosure (e.g., a cell population as described herein) the subject experiences GVHD and relapse-free survival (GFRS) for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, GFRS can refer to survival free of death from any cause, relapse, Grade 3-4 aGVHD (e.g., graded per MAGIC), and moderate to severe cGVHD (e.g., graded per NIH consensus criteria). In some embodiments, relapse for acute leukemia can refer to ≥5% blasts in the bone marrow or peripheral blood, reappearance of pre-transplant cytogenetic abnormality, and/or new evidence or redevelopment of extramedullary disease. In some embodiments, relapse for MDS can refer to satisfying criteria for evolution into acute leukemia, reappearance of pre-transplant morphologic abnormalities, detected in bone marrow specimens, and/or reappearance of pre-transplant cytogenetic abnormality in at least one metaphase on each of two separate consecutive examinations at least one month apart, regardless of the number of metaphases analyzed.

In some embodiments, relapse for CML can refer to the emergence of BCR-ABL positivity and/or cytogenetic relapse that requires the institution of secondary therapy for CML. In some embodiments, this can include the use of donor lymphocyte infusion, tyrosine kinase inhibitors (TKIs), or chemotherapy in response to molecular and/or cytogenetic progression.

In some embodiments, subjects administered a composition of the disclosure (e.g., a cell population as described herein) experience increased relapse-free survival, relative to a corresponding human subject that has been administered a standard myeloablative allogeneic hematopoietic stem cell transplant (alloHSCT). In some embodiments, the subject experiences relapse-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, the subject experiences overall survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, the subject does not experience non-relapse mortality for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, subjects administered a composition of the disclosure (e.g., a cell population as described herein) have decreased incidence of Grade 3 or higher infections, relative to a corresponding human subject that has been administered a standard myeloablative allogeneic hematopoietic stem cell transplant (alloHSCT). In some embodiments, the subject is free of Grade 3 or higher infections for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure. In some embodiments, the Grade 3 or higher infection is graded using the BMT-CTN MOP V4.0 grading scale.

In some embodiments, subjects administered a composition of the disclosure (e.g., a cell population as described herein) have a decreased incidence of re-hospitalization, relative to a corresponding human subject that has been administered a standard myeloablative allogeneic hematopoietic stem cell transplant (alloHSCT). In some embodiments, the subject does not require re-hospitalization for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

Serious infections can occur at alloHCT settings partially due to procedures such as conditioning and administering GVHD prophylactic agents. Opportunistic infections can be severe and lethal. Several infectious disease guidelines specifically for patients with chronic GVHD may be found in the guidelines for preventing opportunistic infections developed by the American Society of Blood and Marrow Transplantation, Centers for Disease Control, and Infectious Disease Society of America, published as a supplement to Biology of Blood and Marrow Transplantation (6a:659-734, 2000) and posted on the CDC website at www.cdc.gov. Infections can be graded based on the Technical Manual of Procedures by the Blood and Marrow Transplant Clinical Trials Network (BMT CTN MOP).

### Engraftment and Immune Reconstitution

### Graft Failure:

Subjects that are administered a composition of the disclosure (e.g., a cell population as described herein) may exhibit a low rate of primary graft failure, for example, a lower rate of primary graft failure compared to subjects that are administered an alternate composition. Primary graft failure can be a failure to achieve an absolute neutrophil count of >500 cells/μL after Day 30 post-transplant. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit primary graft failure. In some embodiments, less than about 1% of subjects administered a composition of the disclosure exhibit primary graft failure. In some embodiments, less than about 3% of subjects administered a composition of the disclosure exhibit primary graft failure.

Subjects that are administered a composition of the disclosure (e.g., a cell population as described herein) may exhibit a low rate of secondary graft failure, for example, a lower rate of secondary graft failure compared to subjects that are administered an alternate composition. Secondary graft failure can be a sustained loss of hematopoiesis after engraftment. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit secondary graft failure when evaluated a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 1% of subjects administered a composition of the disclosure exhibit secondary graft failure within 1-year post-transplant. In some embodiments, less than about 5% of subjects administered a composition of the disclosure exhibit secondary graft failure within 1-year post-transplant.

In some embodiments, subjects administered a composition of the disclosure (e.g., a cell population as described herein) have a decreased wherein incidence of primary graft failure or secondary graft failure, relative to a corresponding human subject that has been administered a standard myeloablative allogeneic hematopoietic stem cell transplant (alloHSCT). In some embodiments, the subject is free of primary graft failure or secondary graft failure for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

### Neutrophil Engraftment:

Subjects that are administered a composition of the disclosure (e.g., a cell population as described herein) may exhibit fast neutrophil engraftment, for example, faster neutrophil engraftment compared to subjects that are administered an alternate composition. Neutrophil engraftment can be indicated by a sustained neutrophil count of >500 cells/μL in the peripheral blood of the recipient. In some embodiments, a population of subjects administered a composition of the disclosure achieve neutrophil engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve neutrophil engraftment by a median of 13 days post-transplant. In some embodiments, a population of subjects administered a composition of the disclosure achieve neutrophil engraftment by a median of 12 days post-transplant. In some embodiments, a population of subjects administered a composition of the disclosure achieve neutrophil engraftment by a median of 11 days post-transplant.

In some embodiments, subjects administered a composition of the disclosure (e.g., a cell population as described herein) have increased incidence, timing, or both incidence and timing of neutrophil engraftment, relative to a corresponding human subject that has been administered a standard myeloablative allogeneic hematopoietic stem cell transplant (alloHSCT). In some embodiments, neutrophil engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, or approximately 90 days or more after administration of the composition of the disclosure.

### Platelet Engraftment:

Subjects that are administered a composition of the disclosure (e.g., a cell population comprising a population of cells described herein) may exhibit fast platelet engraftment, for example, faster platelet engraftment compared to subjects that are administered an alternate composition. Platelet engraftment can be indicated by a platelet count >20,000/mm³ for 3 consecutive days without platelet transfusion in the peripheral blood of the recipient. In some embodiments, a population of subjects administered a composition of the disclosure achieve platelet engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve platelet engraftment by a median of 13 days post-transplant. In some embodiments, a population of subjects administered a composition of the disclosure achieve platelet engraftment by a median of 12 days post-transplant. In some embodiments, a population of subjects administered a composition of the disclosure achieve platelet engraftment by a median of 11 days post-transplant.

In some embodiments, subjects administered a composition of the disclosure (e.g., a cell population as described herein) have increased incidence, timing, or both incidence and timing of platelet engraftment, relative to a corresponding human subject that has been administered a standard myeloablative allogeneic hematopoietic stem cell transplant (alloHSCT). In some embodiments, platelet engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, or approximately 90 days or more after administration of the composition of the disclosure.

### T-Cell Engraftment:

Subjects that are administered a composition of the disclosure (e.g., a cell population as described herein) exhibit a high proportion of circulating Tregs, for example, a higher proportion of circulating Tregs compared to subjects that are administered an alternate composition. In some embodiments, an average of at least about 5%, at least about 7.5%, at least about 10%, at least about 12.5%, at least about 15%, at least about 16%, at least about 17%, at least about 18%, at least about 19%, at least about 20%, at least about 21%, at least about 22%, at least about 23%, at least about 24%, or at least about 25% of circulating CD4⁺ T cells are Tregs when subjects are evaluated a suitable amount of time post-transplant, for example, at approximately 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, or 180 days post-transplant.

Subjects that are administered a composition of the disclosure (e.g., a cell population as described herein) exhibit a normal CD4:CD8 T cell ratio, for example, a higher CD4:CD8 T cell ratio compared to subjects that are administered an alternate composition or normal healthy control subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥0.8, ≥0.9, ≥1, ≥1.1, ≥1.2, ≥1.3, ≥1.4, or ≥1.5 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than approximately 50%, more than approximately 60%, more than approximately 70%, more than approximately 80%, or more than approximately 90% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### B-Cell Engraftment:

Subjects that are administered a composition of the disclosure (e.g., a cell population as described herein) may exhibit a high concentration of circulating B cells at an early timepoint after transplant, for example, a higher concentration of circulating B cells compared to subjects that are administered an alternate composition. Achieving a high concentration of circulating B cells at an early timepoint after transplant can be important for immunocompetence, and can allow vaccines to elicit protective immune responses at an earlier timepoint post-transplant.

In some embodiments, more than about 50 B cells per L, more than about 60 B cells per L, more than about 70 B cells per L, more than about 80 B cells per L, more than about 90 B cells per L, more than about 100 B cells per L, more than about 110 B cells per L, or more than about 120 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

Subjects that are administered a composition of the disclosure (e.g., a cell population as described herein) may exhibit a high proportion of mature B cells at an early timepoint after transplant, for example, a higher proportion of circulating B cells that are mature B cells (e.g., IgD⁺ and/or CD27⁺) compared to subjects that are administered an alternate composition. Achieving a high proportion of mature B cells at an early timepoint after transplant can be important for immunocompetence, and can allow vaccines to elicit protective immune responses at an earlier timepoint post-transplant.

In some embodiments, at least about 50%, at least about 60%, at least about 70%, at least about 80%, or at least about 90% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 20%, at least about 25%, at least about 30%, at least about 35%, at least about 40%, or at least about 45% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

Clinical outcomes disclosed herein can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

### Kits

Another aspect provides a kit, unit dose, article of manufacture, or container that comprises a composition of the present disclosure (e.g., a cellular therapy product or pharmaceutical treatment). In various embodiments, the kit further comprises a GVHD prophylactic agent of the present disclosure. In some embodiments, the kit further comprises instructions for use of the kit in performing any herein-disclosed method.

Another aspect provides a kit, unit dose, article of manufacture, or container that comprises a population of isolated CD45⁺ cells, wherein the population of isolated CD45⁺ cells comprises a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{di}m regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. In some embodiments, the kit further comprises instructions for use of the kit in performing any herein-disclosed method.

Another aspect provides a kit or article of manufacture that comprises a container comprising a population of isolated CD45⁺ cells, wherein the population of isolated CD45⁺ cells comprises a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC); b) a dose of approximately 1.0×10⁵ to approximately 1.0×10⁸ CD45RA⁻ memory T cells (Tmem) per kilogram of body weight of the human subject receiving the product or a total dose of approximately 5.0×10⁵ to approximately 1.5×10¹⁰ CD45RA⁻ memory T cells (Tmem); and c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg), wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and wherein the population of isolated CD45⁺ cells is formulated with an excipient at a neutral pH. In some embodiments, the kit further comprises instructions for use of the kit in performing any herein-disclosed method.

Another aspect provides a kit that comprises a container comprising a solution comprising a population of CD34+ cells, wherein the population is enriched for regulatory T cells (Treg) and for memory T cells (Tmem), and wherein the population is depleted of naïve conventional αβ-T cells or naïve conventional CD3⁺CD25⁻CD45RA⁺ T cells (Tcon). In this aspect, the solution comprising a population of CD34⁺ cells, wherein the population is enriched for regulatory T cells (Treg) and memory T cells (Tmem), and wherein the population is depleted of naïve conventional αβ-T cells or naïve conventional CD3⁺CD25⁻CD45RA⁺ T cells (Tcon) is as defined according to any herein disclosed pharmaceutical treatment or method. In various embodiments, the kit further comprises a container comprising a GVHD prophylactic agent of the present disclosure. In some embodiments, the kit further comprises instructions for performing any herein-disclosed method.

A further aspect provides a kit comprising: (a) one or more reagents to sort CD34⁺ cells from a mobilized peripheral blood composition; (b) one or more reagents to sort regulatory T cells (Treg) from the mobilized peripheral blood composition; (c) one or more reagents to sort memory T cells (Tmem); and (d) one or more reagents to deplete naïve conventional αβ-T cells or naïve conventional CD3⁺CD25⁻CD45RA⁺ T cells (Tcon). In some embodiments the one or more reagents used to the Tmem also deplete the Tcon. In some embodiments, the kit further comprises instructions for making any of the compositions of the present disclosure.

Various embodiments provide a kit comprising one or more reagents for sorting HSPC, for instance, a kit may comprise reagents to enrich a CD34⁺ cell population from a mobilized peripheral blood donation.

Some embodiments provides a kit comprising one or more reagents for sorting Treg, for instance, a kit may comprise reagents to enrich a Treg cell population (using markers as described elsewhere herein) from a mobilized peripheral blood donation.

Some embodiments provide a kit comprising one or more reagents for sorting Tmem, for instance, a kit may comprise reagents to enrich a Tmem cell population (using markers as described elsewhere herein) from a mobilized peripheral blood donation.

Some embodiments provide a kit comprising one or more reagents for depleting Tcon, for instance, a kit may comprise reagents to deplete a cell population of Tcon (using markers as described elsewhere herein) from a mobilized peripheral blood donation.

Embodiments provide a kit comprising one or more conditioning reagents for a conditioning regimen, for instance, a kit may comprise reagents for myeloablation or myeloreduction of a recipient.

Some embodiment provides a kit comprising one or more GVHD prophylactic agents.

In any herein-disclosed method, the method further comprises providing instructions for use (IFU), the IFU including instructions for administering the cell populations to the patient. In some cases, the IFU also include instructions for administering one or more pharmaceutical agents or compositions to the patient.

### ENUMERATED EMBODIMENTS

The following enumerated embodiments describe further non-limiting aspects of the present disclosure.
Embodiment 1. A cellular therapy product comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg),
   wherein the cellular therapy product has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 2. The cellular therapy product of embodiment 1, wherein the dose of HSPC comprises a dose of approximately 1.0×10⁵ or more HSPC per kilogram of body weight of the human subject receiving the product, approximately 2.0×10⁵ or more HSPC per kilogram of body weight of the human subject receiving the product, approximately 3.0×10⁵ or more HSPC per kilogram of body weight of the human subject receiving the product, approximately 4.0×10⁵ or more HSPC per kilogram of body weight of the human subject receiving the product, approximately 5.0×10⁵ or more HSPC per kilogram of body weight of the human subject receiving the product, approximately 6.0×10⁵ or more HSPC per kilogram of body weight of the human subject receiving the product, approximately 7.0×10⁵ or more HSPC per kilogram of body weight of the human subject receiving the product, approximately 8.0×10⁵ or more HSPC per kilogram of body weight of the human subject receiving the product, approximately 9.0×10⁵ or more HSPC per kilogram of body weight of the human subject receiving the product, approximately 1.0×10⁶ or more HSPC per kilogram of body weight of the human subject receiving the product, approximately 1.5×10⁶ or more HSPC per kilogram of body weight of the human subject receiving the product, approximately 2.0×10⁶ or more HSPC per kilogram of body weight of the human subject receiving the product, approximately 2.5×10⁶ or more HSPC per kilogram of body weight of the human subject receiving the product, approximately 3.0×10⁶ or more HSPC per kilogram of body weight of the human subject receiving the product, approximately 3.5×10⁶ or more HSPC per kilogram of body weight of the human subject receiving the product, approximately 4.0×10⁶ or more HSPC per kilogram of body weight of the human subject receiving the product, approximately 4.5×10⁶ or more HSPC per kilogram of body weight of the human subject receiving the product, approximately 5.0×10⁶ or more HSPC per kilogram of body weight of the human subject receiving the product, approximately 5.5×10⁶ or more HSPC per kilogram of body weight of the human subject receiving the product, approximately 6.0×10⁶ or more HSPC per kilogram of body weight of the human subject receiving the product, approximately 6.5×10⁶ or more HSPC per kilogram of body weight of the human subject receiving the product, approximately 7.0×10⁶ or more HSPC per kilogram of body weight of the human subject receiving the product, approximately 7.5×10⁶ or more HSPC per kilogram of body weight of the human subject receiving the product, approximately 8.0×10⁶ or more HSPC per kilogram of body weight of the human subject receiving the product, approximately 8.5×10⁶ or more HSPC per kilogram of body weight of the human subject receiving the product, approximately 9.0×10⁶ or more HSPC per kilogram of body weight of the human subject receiving the product, approximately 9.5×10⁶ or more HSPC per kilogram of body weight of the human subject receiving the product, approximately 1.0×10⁷ or more HSPC per kilogram of body weight of the human subject receiving the product, approximately 1.5×10⁷ or more HSPC per kilogram of body weight of the human subject receiving the product, approximately 2.0×10⁷ or more HSPC per kilogram of body weight of the human subject receiving the product, approximately 2.5×10⁷ or more HSPC per kilogram of body weight of the human subject receiving the product, approximately 3.0×10⁷ or more HSPC per kilogram of body weight of the human subject receiving the product, approximately 3.5×10⁷ or more HSPC per kilogram of body weight of the human subject receiving the product, approximately 4.0×10⁷ or more HSPC per kilogram of body weight of the human subject receiving the product, approximately 4.5×10⁷ or more HSPC per kilogram of body weight of the human subject receiving the product, approximately 5.0×10⁷ or more HSPC per kilogram of body weight of the human subject receiving the product, approximately 5.5×10⁷ or more HSPC per kilogram of body weight of the human subject receiving the product, approximately 6.0×10⁷ or more HSPC per kilogram of body weight of the human subject receiving the product, approximately 6.5×10⁷ or more HSPC per kilogram of body weight of the human subject receiving the product, approximately 7.0×10⁷ or more HSPC per kilogram of body weight of the human subject receiving the product, approximately 7.5×10⁷ or more HSPC per kilogram of body weight of the human subject receiving the product, approximately 8.0×10⁷ or more HSPC per kilogram of body weight of the human subject receiving the product, approximately 8.5×10⁷ or more HSPC per kilogram of body weight of the human subject receiving the product, approximately 9.0×10⁷ or more HSPC per kilogram of body weight of the human subject receiving the product, approximately 9.5×10⁷ or more HSPC per kilogram of body weight of the human subject receiving the product, or approximately 1.0×10⁸ or more HSPC per kilogram of body weight of the human subject receiving the product.
Embodiment 3. The cellular therapy product of embodiment 1 or embodiment 2, wherein the dose of HSPC comprises a total dose of approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC.
Embodiment 4. The cellular therapy product of any one of embodiments 1-3, wherein the dose of Tmem comprises a dose of approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product.
Embodiment 5. The cellular therapy product of any one of embodiments 1-4, wherein the dose of Tmem comprises a total dose of approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem.
Embodiment 6. The cellular therapy product of any one of embodiments 1-5, wherein the dose of fresh Treg comprises a dose of approximately 1.0×10⁵ or more fresh Treg per kilogram of body weight of the human subject receiving the product, approximately 2.0×10⁵ or more fresh Treg per kilogram of body weight of the human subject receiving the product, approximately 3.0×10⁵ or more fresh Treg per kilogram of body weight of the human subject receiving the product, approximately 4.0×10⁵ or more fresh Treg per kilogram of body weight of the human subject receiving the product, approximately 5.0×105 or more fresh Treg per kilogram of body weight of the human subject receiving the product, approximately 6.0×10⁵ or more fresh Treg per kilogram of body weight of the human subject receiving the product, approximately 7.0×10⁵ or more fresh Treg per kilogram of body weight of the human subject receiving the product, approximately 8.0×10⁵ or more fresh Treg per kilogram of body weight of the human subject receiving the product, approximately 9.0×10⁵ or more fresh Treg per kilogram of body weight of the human subject receiving the product, approximately 1.0×10⁶ or more fresh Treg per kilogram of body weight of the human subject receiving the product, approximately 1.5×10⁶ or more fresh Treg per kilogram of body weight of the human subject receiving the product, approximately 2.0×10⁶ or more fresh Treg per kilogram of body weight of the human subject receiving the product, approximately 2.5×10⁶ or more fresh Treg per kilogram of body weight of the human subject receiving the product, approximately 3.0×10⁶ or more fresh Treg per kilogram of body weight of the human subject receiving the product, approximately 3.5×10⁶ or more fresh Treg per kilogram of body weight of the human subject receiving the product, approximately 4.0×10⁶ or more fresh Treg per kilogram of body weight of the human subject receiving the product, approximately 4.5×10⁶ or more fresh Treg per kilogram of body weight of the human subject receiving the product, approximately 5.0×10⁶ or more fresh Treg per kilogram of body weight of the human subject receiving the product, approximately 5.5×10⁶ or more fresh Treg per kilogram of body weight of the human subject receiving the product, approximately 6.0×10⁶ or more fresh Treg per kilogram of body weight of the human subject receiving the product, approximately 6.5×10⁶ or more fresh Treg per kilogram of body weight of the human subject receiving the product, approximately 7.0×10⁶ or more fresh Treg per kilogram of body weight of the human subject receiving the product, approximately 7.5×10⁶ or more fresh Treg per kilogram of body weight of the human subject receiving the product, approximately 8.0×10⁶ or more fresh Treg per kilogram of body weight of the human subject receiving the product, approximately 8.5×10⁶ or more fresh Treg per kilogram of body weight of the human subject receiving the product, approximately 9.0×10⁶ or more fresh Treg per kilogram of body weight of the human subject receiving the product, approximately 9.5×10⁶ or more fresh Treg per kilogram of body weight of the human subject receiving the product, approximately 1.0×10⁷ or more fresh Treg per kilogram of body weight of the human subject receiving the product, approximately 1.5×10⁷ or more fresh Treg per kilogram of body weight of the human subject receiving the product, or approximately 2.0×10⁷ or more fresh Treg per kilogram of body weight of the human subject receiving the product.
Embodiment 7. The cellular therapy product of any one of embodiments 1-6, wherein the dose of fresh Treg comprises a total dose of approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg.
Embodiment 8. The cellular therapy product of any one of embodiments 1-7, wherein the cellular therapy product comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product.
Embodiment 9. The cellular therapy product of any one of embodiments 1-8, wherein the cellular therapy product comprises less than approximately 6.0×10⁷ total Tcon, less than approximately 5.5×10⁷ total Tcon, less than approximately 5.0×10⁷ total Tcon, less than approximately 4.5×10⁷ total Tcon, less than approximately 4.0×10⁷ total Tcon, less than approximately 3.5×10⁷ total Tcon, less than approximately 3.0×10⁷ total Tcon, less than approximately 2.5×10⁷ total Tcon, less than approximately 2.0×10⁷ total Tcon, less than approximately 1.5×10⁷ total Tcon, less than approximately 1.0×10⁷ total Tcon, less than approximately 9.5×10⁶ total Tcon, less than approximately 9.0×10⁶ total Tcon, less than approximately 8.5×10⁶ total Tcon, less than approximately 8.0×10⁶ total Tcon, less than approximately 7.5×10⁶ total Tcon, less than approximately 7.0×10⁶ total Tcon, less than approximately 6.5×10⁶ total Tcon, less than approximately 6.0×10⁶ total Tcon, less than approximately 5.5×10⁶ total Tcon, less than approximately 5.0×10⁶ total Tcon, less than approximately 4.5×10⁶ total Tcon, less than approximately 4.0×10⁶ total Tcon, less than approximately 3.5×10⁶ total Tcon, less than approximately 3.0×10⁶ total Tcon, less than approximately 2.5×10⁶ total Tcon, less than approximately 2.0×10⁶ total Tcon, less than approximately 1.5×10⁶ total Tcon, or less than approximately 1.0×10⁶ total Tcon.
Embodiment 10. The cellular therapy product of any one of embodiments 1-9, wherein the population of isolated CD45+ cells is formulated at a volume that ranges from approximately 5 mL to approximately 1 L.
Embodiment 11. The cellular therapy product of embodiment 10, wherein the population of isolated CD45+ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L.
Embodiment 12. The cellular therapy product of any one of embodiments 1-11, wherein the neutral pH ranges from approximately 6.8 to approximately 7.6.
Embodiment 13. The cellular therapy product of any one of embodiments 1-12, wherein the excipient comprises a transport buffer.
Embodiment 14. The cellular therapy product of any one of embodiments 1-13, wherein the transport buffer comprises approximately 120 to approximately 160 mEq sodium.
Embodiment 15. The multi-component cellular therapy product of embodiment 13 or embodiment 14, wherein the transport buffer comprises approximately 270 to approximately 320 mOsmol/L total.
Embodiment 16. The cellular therapy product of any one of embodiments 13-15, wherein the transport buffer is selected from the group consisting of: phosphate-buffered saline (PBS), human serum, PlasmaLyte, Normosol-R, and any combination thereof.
Embodiment 17. The cellular therapy product of any one of embodiments 13-16, wherein the transport buffer further comprises approximately 0.1% volume by volume to approximately 10% volume by volume of a human carrier protein.
Embodiment 18. The cellular therapy product of embodiment 17, wherein the human carrier protein is selected from the group consisting of: human serum albumin (HSA), intravenous immune globulin (IVIG), AB serum, and any combination thereof.
Embodiment 19. The cellular therapy product of any one of embodiments 1-18, wherein the population of isolated CD45+ cells is formulated in a single dose transfer bag.
Embodiment 20. The cellular therapy product of embodiment 19, wherein the single dose transfer bag is a polyvinyl chloride (PVC) transfer bag or an ethylene vinyl acetate (EVA) transfer bag.
Embodiment 21. The cellular therapy product of any one of embodiments 1-20, wherein the population of isolated CD45+ cells is enriched for HSPC, Tmem, and Treg.
Embodiment 22. The cellular therapy product of any one of embodiments 1-21, wherein the population of isolated CD45+ cells comprises a ratio of Tcon to HSPC that is less than 1:3, less than 1:5, less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, less than 1:50,000, less than 1:55,000, less than 1:60,000, less than 1:65,000, less than 1:70,000, less than 1:75,000, less than 1:80,000, less than 1:85,000, less than 1:90,000, less than 1:95,000, less than 1:100,000, less than 1:200,000, less than 1:300,000, less than 1:400,000, less than 1:500,000, less than 1:600,000, less than 1:700,000, less than 1:800,000, less than 1:900,000, or less than 1:1,000,000.
Embodiment 23. The cellular therapy product of any one of embodiments 1-22, wherein the population of isolated CD45+ cells comprises a ratio of Tcon to Tmem this is less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000.
Embodiment 24. The cellular therapy product of any one of embodiments 1-23, wherein the population of isolated CD45+ cells comprises a ratio of Tcon to Treg that is less than 1:1, less than 1:2, less than 1:3, less than 1:4, less than 1:5, less than 1:6, less than 1:7, less than 1:8, less than 1:9, less than 1:10, less than 1:11, less than 1:12, less than 1:13, less than 1:14, less than 1:15, less than 1:16, less than 1:17, less than 1:18, less than 1:19, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000.
Embodiment 25. The cellular therapy product of any one of embodiments 1-24, wherein the population of isolated CD45+ cells comprises a ratio of HSPC to Tmem that is approximately 500:1 to approximately 1:1,000.
Embodiment 26. The cellular therapy product of any one of embodiments 1-25, wherein the population of isolated CD45+ cells comprises a ratio of HSPC to Treg that is from approximately 100:1 to approximately 1:30.
Embodiment 27. The cellular therapy product of any one of embodiments 1-26, wherein the population of isolated CD45+ cells comprises a ratio of Tmem to Treg that is from approximately 2000:1 to approximately 1:10.
Embodiment 28. The cellular therapy product of any one of embodiments 1-27, wherein the population of isolated CD45+ cells further comprises an enriched population of TCR Vα24Jα18+CD127+ invariant Natural Killer T cells (iNKT).
Embodiment 29. The cellular therapy product of embodiment 28, wherein the population of isolated CD45+ cells comprises a dose of approximately 5.0×10² to approximately 2.0×10⁶ iNKT per kilogram of body weight of the human subject receiving the product or a total dose of approximately 2.0×10³ to approximately 4.0×10⁸ iNKT.
Embodiment 30. The cellular therapy product of any one of embodiment 28 or embodiment 29, wherein the dose of iNKT comprises a dose of approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product.
Embodiment 31. The cellular therapy product of any one of embodiments 28-30, wherein the dose of iNKT comprises a total dose of approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT.
Embodiment 32. The cellular therapy product of any one of embodiments 28-31, wherein the population of isolated CD45+ cells comprises a ratio of HSPC to iNKT that is from approximately 1:2 to approximately 500,000:1.
Embodiment 33. The cellular therapy product of any one of embodiments 28-32, wherein the population of isolated CD45+ cells comprises a ratio of iNKT to Tcon is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100.
Embodiment 34. The cellular therapy product of any one of embodiments 28-33, wherein the population of isolated CD45+ cells comprises a ratio of iNKT to Tmem that is from approximately 5:1 to approximately 1:1,000,000.
Embodiment 35. The cellular therapy product of any one of embodiments 1-34, wherein the HSPC are cKIT+, CD133+, CD90+, CD38−, CD45RA−, Lin−, CD19−, TCRα−, or any combination thereof.
Embodiment 36. The cellular therapy product of any one of embodiments 1-35, wherein the Tmem are CD3+CD45RO+.
Embodiment 37. The cellular therapy product of any one of embodiments 1-36, wherein the Tmem comprise a population of T central memory cells (TCM), a population of T effector memory cells (TEM), a population of T stem central memory cells (TSCM), or any combination thereof.
Embodiment 38. The cellular therapy product of embodiment 37, wherein the TCM are CD4+CD45RO+ or CD8+CD45RO+.
Embodiment 39. The cellular therapy product of embodiment 37 or embodiment 38, wherein the TCM are CD45RA−, CD62L+, CCR7+, or any combination thereof.
Embodiment 40. The cellular therapy product of any one of embodiments 37-39, wherein the TEM are CD4+, CD45RO+, CD45RA−, CD62L−, CCR7−, or any combination thereof.
Embodiment 41. The cellular therapy product of any one of embodiments 37-40, wherein the TSCM are CD4+CD45RA+ or CD8+CD45RA+.
Embodiment 42. The cellular therapy product of any one of embodiments 37-41, wherein the TSCM are CD95+, CD122+, CXCR3+, LFA-1+, or any combination thereof.
Embodiment 43. The cellular therapy product of any one of embodiments 1-42, wherein the Treg are FoxP3+.
Embodiment 44. The cellular therapy product of any one of embodiments 1-43, wherein the Treg comprise a population of naïve Treg cells, a population of memory Treg cells, or a population of naïve Treg cells and memory Treg cells.
Embodiment 45. The cellular therapy product of embodiment 44, wherein the naïve Treg cells are CD4+CD25+CD127dimFoxP3+CD45RA+CD45RO−.
Embodiment 46. The cellular therapy product of embodiment 44 or embodiment 45, wherein the dose of Treg comprises a dose of approximately 2.0×10⁵ to approximately 5.0×10⁸ naïve Treg cells per kilogram of body weight of the human subject receiving the product, or a total dose of approximately 8.0×10⁵ to approximately 1.0×10¹¹ naïve Treg cells.
Embodiment 47. The cellular therapy product of any one of embodiments 44-46, wherein the population of isolated CD45+ cells comprises a ratio of HSPC to naïve Treg that is from approximately 1:500 to approximately 100:1.
Embodiment 48. The cellular therapy product of any one of embodiments 44-47, wherein the population of isolated CD45+ cells comprises a ratio of Tmem to naïve Treg that is from approximately 3:1 to approximately 1:10.
Embodiment 49. The cellular therapy product of any one of embodiments 44-48, wherein the population of isolated CD45+ cells comprises a ratio of Tcon to naïve Treg that is less than 1:1, less than 1:2, less than 1:3, less than 1:4, less than 1:5, less than 1:6, less than 1:7, less than 1:8, less than 1:9, or less than 1:10.
Embodiment 50. The cellular therapy product of any one of embodiments 44-49, wherein the memory Treg cells are CD4+CD25+CD127dimFoxP3+CD45RA-CD45RO+.
Embodiment 51. The cellular therapy product of any one of embodiments 44-50, wherein the dose of Treg comprises a dose of approximately 5.0×10⁴ to approximately 5.0×10⁸ memory Treg cells per kilogram of body weight of the human subject receiving the product, or a total dose of approximately 2.0×10⁵ to approximately 1.0×10¹¹ memory Treg cells.
Embodiment 52. The cellular therapy product of any one of embodiments 44-51, wherein the population of isolated CD45+ cells comprises a ratio of HSPC to memory Treg that is from approximately 1:500 to approximately 10,000:1.
Embodiment 53. The cellular therapy product of any one of embodiments 44-52, wherein the population of isolated CD45+ cells comprises a ratio of Tmem to memory Treg that is from approximately 27:1 to approximately 9:10.
Embodiment 54. The cellular therapy product of any one of embodiments 44-53, wherein the population of isolated CD45+ cells comprises a ratio of Tcon to memory Treg is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100.
Embodiment 55. The cellular therapy product of any one of embodiments 1-54, wherein the Tcon are TCRα+, TCRβ+, or TCRα+TCRβ+.
Embodiment 56. The cellular therapy product of any one of embodiments 1-55, wherein the Tcon are CD25−, CD127+, or CD25−CD127+.
Embodiment 57. The cellular therapy product of embodiment 56, wherein the Tcon are TCRα+TCRD+CD45RA+CD45RO−CD25−CD95−IL-2Rβ−CD127+.
Embodiment 58. The cellular therapy product of any one of embodiments 28-57, wherein the iNKT are CD1d-tet+, 6B11+, or CD1d-tet+6B11+.
Embodiment 59. The cellular therapy product of any one of embodiments 1-58, wherein the product further comprises a pharmaceutical composition comprising one or more doses of a graft vs host disease (GVHD) prophylactic agent.
Embodiment 60. The cellular therapy product of embodiment 59, wherein the GVHD prophylactic agent is tacrolimus.
Embodiment 61. The cellular therapy product of embodiment 60, wherein one or more doses of tacrolimus are provided to maintain a trough blood level of approximately 5 ng/mL to approximately 10 ng/mL in the human subject receiving the product.
Embodiment 62. The cellular therapy product of embodiment 60 or embodiment 61, wherein the pharmaceutical composition comprises tacrolimus at a dose that ranges from approximately 0.01 mg per kilogram of body weight of the human subject receiving the product to approximately 0.50 mg per kilogram of body weight of the human subject receiving the product twice per day.
Embodiment 63. The cellular therapy product of embodiment 61 or embodiment 62, wherein the tacrolimus is dosed in an amount to maintain or that maintains a target blood level of approximately 1 ng/mL to approximately 10 ng/mL in the human subject receiving the product for approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the population of isolated CD45+ cells.
Embodiment 64. The cellular therapy product of any one of embodiments 1-63, wherein the population of isolated CD45+ cells comprises from less than approximately 5 EU to less than approximately 0.5 EU of endotoxins per mL of formulation.
Embodiment 65. The cellular therapy product of any one of embodiments 1-64, wherein the HSPC, the Tmem, the Treg, or any combination thereof are obtained from an allogeneic HLA-matched donor, relative to the human subject receiving the product.
Embodiment 66. The cellular therapy product of any one of embodiments 1-65, wherein the population of isolated CD45+ cells is from an allogeneic HLA-matched donor, relative to the human subject receiving the product.
Embodiment 67. The cellular therapy product of any one of embodiments 1-66, wherein the HSPC, the Tmem, the Treg, or any combination thereof are obtained from an allogeneic donor having at least one HLA mismatch, relative to the human subject receiving the product.
Embodiment 68. The cellular therapy product of any one of embodiments 1-67, wherein the population of isolated CD45+ cells is from an allogeneic donor having at least one HLA mismatch, relative to the human subject receiving the product.
Embodiment 69. The cellular therapy product of embodiment 67 or embodiment 68, wherein the at least one HLA mismatch is at an allele selected from the group consisting of: HLA-A, HLA-B, HLA-C, HLA-DRB1, and any combination thereof.
Embodiment 70. The cellular therapy product of any one of embodiments 67-69, wherein the cells having at least one HLA mismatch are from a donor that is 6/8 HLA-mismatched, relative to the human subject receiving the product, or is 7/8 HLA-mismatched, relative to the human subject receiving the product.
Embodiment 71. The cellular therapy product of embodiment 70, wherein the cells having at least one HLA mismatch are from a donor that is 7/8 HLA-mismatched, relative to the human subject receiving the product.
Embodiment 72. The cellular therapy product of embodiment 71, wherein the donor that is 7/8 HLA-mismatched, relative to the human subject receiving the product, has a mismatch in HLA-A.
Embodiment 73. The cellular therapy product of embodiment 71, wherein the donor that is 7/8 HLA-mismatched, relative to the human subject receiving the product, has a mismatch in HLA-B.
Embodiment 74. The cellular therapy product of embodiment 71, wherein the donor that is 7/8 HLA-mismatched, relative to the human subject receiving the product, has a mismatch in HLA-C.
Embodiment 75. The cellular therapy product of embodiment 71, wherein the donor that is 7/8 HLA-mismatched, relative to the human subject receiving the product, has a mismatch in HLA-DRB1.
Embodiment 76. The cellular therapy product of any one of embodiments 67-75, wherein the donor that has the at least one HLA mismatch, relative to the human subject receiving the product, has a mismatched HLA allele as a result of the donor being homozygous for the HLA allele while the human subject receiving the product is heterogeneous for the HLA allele.
Embodiment 77. The cellular therapy product of any one of embodiments 67-75, wherein the donor that has at least one HLA mismatch, relative to the human subject receiving the product, has a mismatched HLA allele as a result of the donor being heterozygous for the HLA allele while the human subject receiving the product is homozygous for the HLA allele.
Embodiment 78. The cellular therapy product of any one of embodiments 67-75, wherein the donor that has at least one HLA mismatch, relative to the human subject receiving the product, has a mismatched HLA allele as a result of both the donor and the human subject receiving the product being heterozygous for the HLA allele.
Embodiment 79. The cellular therapy product of any one of embodiments 1-78, wherein the HSPC, the Tmem, the Treg, or any combination thereof are obtained from an allogeneic haploidentical donor, relative to the human subject receiving the product.
Embodiment 80. The cellular therapy product of any one of embodiments 1-79, wherein the population of isolated CD45+ cells is from an allogeneic haploidentical donor, relative to the human subject receiving the product.
Embodiment 81. The cellular therapy product of any one of embodiments 65-80, wherein the allogeneic donor is related to the human subject receiving the product.
Embodiment 82. The cellular therapy product of any one of embodiments 65-80, wherein the allogeneic donor is unrelated to the human subject receiving the product.
Embodiment 83. The cellular therapy product of any one of embodiments 1-82, wherein the body weight of the human subject receiving the product is actual body weight.
Embodiment 84. The cellular therapy product of any one of embodiments 1-82, wherein the body weight of the human subject receiving the product is ideal body weight.
Embodiment 85. The cellular therapy product of any one of embodiments 1-84 for use in a method of treating a human subject having or suspected of having a hematologic malignancy.
Embodiment 86. The cellular therapy product of embodiment 85, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).
Embodiment 87. The cellular therapy product of any one of embodiments 1-84 for use in a method of treating a human subject having or suspected of having an autoimmune disorder.
Embodiment 88. The cellular therapy product of embodiment 87, wherein the autoimmune disorder is multiple sclerosis.
Embodiment 89. A method of treating a human subject having or suspected of having acute leukemia in complete remission, the method comprising administering to the human subject a cellular therapy product comprising a population of isolated CD45+ cells, wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 90. A method of treating a human subject having or suspected of having acute leukemia in complete remission, the method comprising administering to the human subject the cellular therapy product of any one of embodiments 1-84.
Embodiment 91. The method of embodiment 89 or embodiment 90, wherein the human subject has approximately 5% or less blast burden in their bone marrow.
Embodiment 92. The method of any one of embodiments 89-91, wherein there is an absence of circulating blasts and/or blasts with Auer rods in the human subject.
Embodiment 93. The method of any one of embodiments 89-92, wherein there is an absence of extramedullary disease in the human subject.
Embodiment 94. The method of any one of embodiments 89-93, wherein the human subject has an absolute neutrophil count (ANC) that is approximately 1.0×10⁹/L or higher.
Embodiment 95. The method of any one of embodiments 89-94, wherein the human subject has a platelet count that is approximately 1.0×10¹¹/L or higher.
Embodiment 96. The method of any one of embodiments 89-95, wherein the human subject is red blood cell-transfusion-independent.
Embodiment 97. The method of any one of embodiments 89-96, wherein the acute leukemia is in complete remission with incomplete hematologic recovery.
Embodiment 98. The method of embodiment 97, wherein the human subject has residual neutropenia.
Embodiment 99. The method of embodiment 98, wherein the human subject has an absolute neutrophil count (ANC) that is approximately 1.0×10⁹/L or lower.
Embodiment 100. The method of any one of embodiments 89-99, wherein the human subject has residual thrombocytopenia.
Embodiment 101. The method of embodiment 100, wherein the human subject has a platelet count that is approximately 1.0×10¹¹/L or lower.
Embodiment 102. A method of treating a human subject having or suspected of having active acute leukemia, the method comprising administering to the human subject a cellular therapy product comprising a population of isolated CD45+ cells,
wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 103. A method of treating a human subject having or suspected of having active acute leukemia, the method comprising administering to the human subject the cellular therapy product of any one of embodiments 1-84.
Embodiment 104. The method of embodiment 102 or embodiment 103, wherein the human subject has approximately 10% or less leukemic blast infiltration of bone marrow.
Embodiment 105. The method of any one of embodiments 90-105, wherein the acute leukemia is acute myeloid leukemia (AML), acute lymphoid leukemia (ALL), and/or mixed phenotype acute leukemia (MPAL).
Embodiment 106. A method of treating a human subject having or suspected of having chronic myelogenous leukemia (CML), the method comprising administering to the human subject a cellular therapy product comprising a population of isolated CD45+ cells,
wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naive conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 107. A method of treating a human subject having or suspected of having chronic myelogenous leukemia (CML), the method comprising administering to the human subject the cellular therapy product of any one of embodiments 1-84.
Embodiment 108. The method of embodiment 106 or embodiment 107, wherein the human subject is in myeloid blast crises and/or lymphoid blast crisis.
Embodiment 109. The method of embodiment 108, wherein the blast crisis is in complete remission.
Embodiment 110. The method of embodiment 108, wherein the blast crisis is in complete remission with incomplete hematologic recovery.
Embodiment 111. The method of any one of embodiments 106-110, wherein the CML is in accelerated phase.
Embodiment 112. The method of any one of embodiments 106-110, wherein the CML is in chronic phase.
Embodiment 113. The method of embodiment 112, wherein the chronic phase CML is resistant to or intolerant of one or more first-generation or second-generation tyrosine kinase inhibitors (TKIs).
Embodiment 114. A method of treating a human subject having or suspected of having high-risk or very high-risk myelodysplastic syndrome (MDS), the method comprising administering to the human subject a cellular therapy product comprising a population of isolated CD45+ cells, wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 115. A method of treating a human subject having or suspected of having high-risk or very high-risk myelodysplastic syndrome (MDS), the method comprising administering to the human subject the cellular therapy product of any one of embodiments 1-84.
Embodiment 116. The method of embodiment 114 or embodiment 115, wherein the human subject has approximately 10% or less blast burden in their bone marrow.
Embodiment 117. A method of treating a human subject having or suspected of having myelofibrosis, the method comprising administering to the human subject a cellular therapy product comprising a population of isolated CD45+ cells, wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 118. A method of treating a human subject having or suspected of having myelofibrosis, the method comprising administering to the human subject the cellular therapy product of any one of embodiments 1-84.
Embodiment 119. The method of embodiment 117 or embodiment 118, wherein the myelofibrosis is eligible for standard myeloablative allogeneic hematopoietic stem cell transplant (alloHSCT).
Embodiment 120. The method of any one of embodiments 117-119, wherein the myelofibrosis is intermediate-2-risk myelofibrosis or high-risk myelofibrosis.
Embodiment 121. The method of any one of embodiments 117-120, wherein the myelofibrosis is intermediate-1-risk myelofibrosis associated with high symptom burden, low platelet counts, and/or complex cytogenetics.
Embodiment 122. A method of treating a human subject having or suspected of having a hematologic malignancy, the method comprising administering to the human subject a cellular therapy product comprising a population of isolated CD45+ cells, wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 123. The method of embodiment 122, wherein the cellular therapy product comprises the cellular therapy product of any one of embodiments 1-84.
Embodiment 124. The method of embodiment 122 or embodiment 123, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).
Embodiment 125. The method of any one of embodiments 89-124, wherein the method further comprises administering one or more doses of a graft vs host disease (GVHD) prophylactic agent.
Embodiment 126. The method of embodiment 125, wherein the GVHS prophylactic agent is tacrolimus.
Embodiment 127. The method of embodiment 126, wherein the tacrolimus is provided in an amount sufficient to maintain a trough blood level of approximately 5 ng/mL to approximately 10 ng/mL in the human subject.
Embodiment 128. The method of embodiment 126 or embodiment 127, wherein the tacrolimus is provided at a dose that ranges from approximately 0.01 mg per kilogram of body weight of the human subject to 0.50 mg per kilogram of body weight of the human subject twice per day.
Embodiment 129. The method of any one of embodiments 126-128, wherein the tacrolimus is dosed in an amount to maintain or that maintains a target blood level of approximately 1 ng/mL to approximately 10 ng/mL in the human subject for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45+ cells.
Embodiment 130. The method of any one of embodiments 126-129, wherein the tacrolimus is initially administered from approximately 12 hours to approximately 24 hours after administration of the cellular therapy product.
Embodiment 131. The method of any one of embodiments 126-130, wherein administration of the tacrolimus is tapered starting at approximately 90 days after initial administration of the tacrolimus.
Embodiment 132. The method of any one of embodiments 89-131, wherein the administering comprises infusing into the human subject the population of isolated CD45+ cells.
Embodiment 133. The method of any one of embodiments 89-132, wherein the population of isolated CD45+ cells comprises from approximately 1.0×10⁵ to approximately 1.0×10⁸ HSPC per kilogram of body weight of the human subject, or from approximately 5.0×10⁵ to approximately 1.5×10¹⁰ HSPC.
Embodiment 134. The method of any one of embodiments 89-133, wherein the population of isolated CD45+ cells comprises approximately 1.0×10⁵ or more HSPC per kilogram of body weight of the human subject, approximately 2.0×10⁵ or more HSPC per kilogram of body weight of the human subject, approximately 3.0×10⁵ or more HSPC per kilogram of body weight of the human subject, approximately 4.0×10⁵ or more HSPC per kilogram of body weight of the human subject, approximately 5.0×10⁵ or more HSPC per kilogram of body weight of the human subject, approximately 6.0×10⁵ or more HSPC per kilogram of body weight of the human subject, approximately 7.0×10⁵ or more HSPC per kilogram of body weight of the human subject, approximately 8.0×10⁵ or more HSPC per kilogram of body weight of the human subject, approximately 9.0×10⁵ or more HSPC per kilogram of body weight of the human subject, approximately 1.0×10⁶ or more HSPC per kilogram of body weight of the human subject, approximately 1.5×10⁶ or more HSPC per kilogram of body weight of the human subject, approximately 2.0×10⁶ or more HSPC per kilogram of body weight of the human subject, approximately 2.5×10⁶ or more HSPC per kilogram of body weight of the human subject, approximately 3.0×10⁶ or more HSPC per kilogram of body weight of the human subject, approximately 3.5×10⁶ or more HSPC per kilogram of body weight of the human subject, approximately 4.0×10⁶ or more HSPC per kilogram of body weight of the human subject, approximately 4.5×10⁶ or more HSPC per kilogram of body weight of the human subject, approximately 5.0×10⁶ or more HSPC per kilogram of body weight of the human subject, approximately 5.5×10⁶ or more HSPC per kilogram of body weight of the human subject, approximately 6.0×10⁶ or more HSPC per kilogram of body weight of the human subject, approximately 6.5×10⁶ or more HSPC per kilogram of body weight of the human subject, approximately 7.0×10⁶ or more HSPC per kilogram of body weight of the human subject, approximately 7.5×10⁶ or more HSPC per kilogram of body weight of the human subject, approximately 8.0×10⁶ or more HSPC per kilogram of body weight of the human subject, approximately 8.5×10⁶ or more HSPC per kilogram of body weight of the human subject, approximately 9.0×10⁶ or more HSPC per kilogram of body weight of the human subject, approximately 9.5×10⁶ or more HSPC per kilogram of body weight of the human subject, approximately 1.0×10⁷ or more HSPC per kilogram of body weight of the human subject, approximately 1.5×10⁷ or more HSPC per kilogram of body weight of the human subject, approximately 2.0×10⁷ or more HSPC per kilogram of body weight of the human subject, approximately 2.5×10⁷ or more HSPC per kilogram of body weight of the human subject, approximately 3.0×10⁷ or more HSPC per kilogram of body weight of the human subject, approximately 3.5×10⁷ or more HSPC per kilogram of body weight of the human subject, approximately 4.0×10⁷ or more HSPC per kilogram of body weight of the human subject, approximately 4.5×10⁷ or more HSPC per kilogram of body weight of the human subject, approximately 5.0×10⁷ or more HSPC per kilogram of body weight of the human subject, approximately 5.5×10⁷ or more HSPC per kilogram of body weight of the human subject, approximately 6.0×10⁷ or more HSPC per kilogram of body weight of the human subject, approximately 6.5×10⁷ or more HSPC per kilogram of body weight of the human subject, approximately 7.0×10⁷ or more HSPC per kilogram of body weight of the human subject, approximately 7.5×10⁷ or more HSPC per kilogram of body weight of the human subject, approximately 8.0×10⁷ or more HSPC per kilogram of body weight of the human subject, approximately 8.5×10⁷ or more HSPC per kilogram of body weight of the human subject, approximately 9.0×10⁷ or more HSPC per kilogram of body weight of the human subject, approximately 9.5×10⁷ or more HSPC per kilogram of body weight of the human subject, or approximately 1.0×10⁸ or more HSPC per kilogram of body weight of the human subject.
Embodiment 135. The method of any one of embodiments 89-134, wherein the population of isolated CD45+ cells comprises approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC.
Embodiment 136. The method of any one of embodiments 89-135, wherein the population of isolated CD45+ cells comprises from approximately 1.0×10⁵ to approximately 1.0×10⁸ Tmem per kilogram of body weight of the human subject receiving the product from approximately 5.0×10⁵ to approximately 1.5×10¹⁰ Tmem.
Embodiment 137. The method of any one of embodiments 89-136, wherein the population of isolated CD45+ cells comprises approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product.
Embodiment 138. The method of any one of embodiments 89-137, wherein the population of isolated CD45+ cells comprises approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem.
Embodiment 139. The method of any one of embodiments 89-138, wherein the population of isolated CD45+ cells comprises from approximately 1.0×10⁵ to approximately 2.0×10⁷ fresh Treg per kilogram of body weight of the human subject or from approximately 5.0×10⁵ to approximately 3.0×10⁹ fresh Treg.
Embodiment 140. The method of any one of embodiments 89-139, wherein the population of isolated CD45+ cells comprises approximately 1.0×10⁵ or more fresh Treg per kilogram of body weight of the human subject, approximately 2.0×10⁵ or more fresh Treg per kilogram of body weight of the human subject, approximately 3.0×10⁵ or more fresh Treg per kilogram of body weight of the human subject, approximately 4.0×10⁵ or more fresh Treg per kilogram of body weight of the human subject, approximately 5.0×10⁵ or more fresh Treg per kilogram of body weight of the human subject, approximately 6.0×10⁵ or more fresh Treg per kilogram of body weight of the human subject, approximately 7.0×10⁵ or more fresh Treg per kilogram of body weight of the human subject, approximately 8.0×10⁵ or more fresh Treg per kilogram of body weight of the human subject, approximately 9.0×10⁵ or more fresh Treg per kilogram of body weight of the human subject, approximately 1.0×10⁶ or more fresh Treg per kilogram of body weight of the human subject, approximately 1.5×10⁶ or more fresh Treg per kilogram of body weight of the human subject, approximately 2.0×10⁶ or more fresh Treg per kilogram of body weight of the human subject, approximately 2.5×10⁶ or more fresh Treg per kilogram of body weight of the human subject, approximately 3.0×10⁶ or more fresh Treg per kilogram of body weight of the human subject, approximately 3.5×10⁶ or more fresh Treg per kilogram of body weight of the human subject, approximately 4.0×10⁶ or more fresh Treg per kilogram of body weight of the human subject, approximately 4.5×10⁶ or more fresh Treg per kilogram of body weight of the human subject, approximately 5.0×10⁶ or more fresh Treg per kilogram of body weight of the human subject, approximately 5.5×10⁶ or more fresh Treg per kilogram of body weight of the human subject, approximately 6.0×10⁶ or more fresh Treg per kilogram of body weight of the human subject, approximately 6.5×10⁶ or more fresh Treg per kilogram of body weight of the human subject, approximately 7.0×10⁶ or more fresh Treg per kilogram of body weight of the human subject, approximately 7.5×10⁶ or more fresh Treg per kilogram of body weight of the human subject, approximately 8.0×10⁶ or more fresh Treg per kilogram of body weight of the human subject, approximately 8.5×10⁶ or more fresh Treg per kilogram of body weight of the human subject, approximately 9.0×10⁶ or more fresh Treg per kilogram of body weight of the human subject, approximately 9.5×10⁶ or more fresh Treg per kilogram of body weight of the human subject, approximately 1.0×10⁷ or more fresh Treg per kilogram of body weight of the human subject, approximately 1.5×10⁷ or more fresh Treg per kilogram of body weight of the human subject, or approximately 2.0×10⁷ or more fresh Treg per kilogram of body weight of the human subject.
Embodiment 141. The method of any one of embodiments 89-140, wherein the population of isolated CD45+ cells comprises approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg.
Embodiment 142. The method of any one of embodiments 89-141, wherein the population of isolated CD45+ cells comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product.
Embodiment 143. The method of any one of embodiments 89-142, wherein the population of isolated CD45+ cells comprises less than approximately 6.0×10⁷ Tcon, less than approximately 5.5×10⁷ Tcon, less than approximately 5.0×10⁷ Tcon, less than approximately 4.5×10⁷ Tcon, less than approximately 4.0×10⁷ Tcon, less than approximately 3.5×10⁷ Tcon, less than approximately 3.0×10⁷ Tcon, less than approximately 2.5×10⁷ Tcon, less than approximately 2.0×10⁷ Tcon, less than approximately 1.5×10⁷ Tcon, less than approximately 1.0×10⁷ Tcon, less than approximately 9.5×10⁶ Tcon, less than approximately 9.0×10⁶ Tcon, less than approximately 8.5×10⁶ Tcon, less than approximately 8.0×10⁶ Tcon, less than approximately 7.5×10⁶ Tcon, less than approximately 7.0×10⁶ Tcon, less than approximately 6.5×10⁶ Tcon, less than approximately 6.0×10⁶ Tcon, less than approximately 5.5×10⁶ Tcon, less than approximately 5.0×10⁶ Tcon, less than approximately 4.5×10⁶ Tcon, less than approximately 4.0×10⁶ Tcon, less than approximately 3.5×10⁶ Tcon, less than approximately 3.0×10⁶ Tcon, less than approximately 2.5×10⁶ Tcon, less than approximately 2.0×10⁶ Tcon, less than approximately 1.5×10⁶ Tcon, or less than approximately 1.0×10⁶ Tcon.
Embodiment 144. The method of any one of embodiments 89-143, wherein the population of isolated CD45+ cells further comprises an enriched population of TCR Vα24Jα18+CD127+ invariant Natural Killer T cells (iNKT).
Embodiment 145. The method of embodiment 144, wherein the population of isolated CD45+ cells comprises from approximately 5.0×10² to approximately 2.0×10⁶ iNKT per kilogram of body weight of the human subject receiving the product or from approximately 2.0×10³ to approximately 4.0×10⁸ iNKT.
Embodiment 146. The method of embodiment 144 or embodiment 145, wherein the population of isolated CD45+ cells comprises approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product.
Embodiment 147. The method of any one of embodiments 144-146, wherein the population of isolated CD45+ cells comprises approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT.
Embodiment 148. The method of any one of embodiments 89-147, wherein the population of isolated CD45+ cells is from an allogeneic donor having at least one HLA mismatch relative to the human subject.
Embodiment 149. The method of any one of embodiments 89-148, wherein the HSPC are cKIT+, CD133+, CD90+, CD38−, CD45RA−, Lin−, CD19−, TCRα-, or any combination thereof.
Embodiment 150. The method of any one of embodiments 89-149, wherein the Tmem are CD3+CD45RO+.
Embodiment 151. The method of any one of embodiments 89-150, wherein the Tmem comprise a population of T central memory cells (TCM), a population of T effector memory cells (TEM), a population of T stem central memory cells (TSCM), or any combination thereof.
Embodiment 152. The method embodiment 151, wherein the TCM are CD4+CD45RO+ or CD8+CD45RO+.
Embodiment 153. The method embodiment 151 or embodiment 152, wherein the TCM are CD45RA−, CD62L+, CCR7+, or any combination thereof.
Embodiment 154. The method of embodiment 151-153, wherein the TEM are CD4+, CD45RO+, CD45RA−, CD62L−, CCR7−, or any combination thereof.
Embodiment 155. The method of embodiment 151-154, wherein the TSCM are CD4+CD45RA+ or CD8+CD45RA+.
Embodiment 156. The method of embodiment 151-155, wherein the TSCM are CD95+, CD122+, CXCR3+, LFA-1+, or any combination thereof.
Embodiment 157. The method of any one of embodiments 89-156, wherein the Treg are FOXP3+.
Embodiment 158. The method of any one of embodiments 89-157, wherein the Treg comprise a population of naïve Treg cells, a population of memory Treg cells, or a population of naïve Treg cells and memory Treg cells.
Embodiment 159. The method of embodiment 158, wherein the naïve Treg cells are CD4+CD25+CD127dimFoxP3+CD45RA+CD45RO−.
Embodiment 160. The method of embodiment 158 or embodiment 159, wherein the Treg comprise from approximately 2.0×10⁵ to approximately 5.0×10⁸ naïve Treg cells per kilogram of body weight of the human subject receiving the product, or from approximately 8.0×10⁵ to approximately 1.0×10¹¹ naïve Treg cells.
Embodiment 161. The method of embodiment 158-160, wherein the memory Treg cells are CD4+CD25+CD127dimFoxP3+CD45RA-CD45RO+.
Embodiment 162. The method of embodiment 158-161, wherein the Treg comprise from approximately 5.0×10⁴ to approximately 5.0×10⁸ memory Treg cells per kilogram of body weight of the human subject receiving the product, or from approximately 2.0×10⁵ to approximately 1.0×10¹¹ memory Treg cells.
Embodiment 163. The method of any one of embodiments 89-162, wherein the Tcon are TCRα+, TCRD+, or TCRα+TCRD+.
Embodiment 164. The method of any one of embodiments 89-163, wherein the Tcon are CD25⁻, CD127+, or CD25⁻CD127+.
Embodiment 165. The method of any one of embodiments 89-164, wherein the Tcon are TCRα+TCRD+CD45RA+CD45RO−CD25−CD95−IL-2Rβ-CD127+.
Embodiment 166. The method of embodiment 144-147, wherein the iNKT are CD1d-tet+, 6B11+, or CD1d-tet+6B11+.
Embodiment 167. The method of any one of embodiments 89-166, wherein the population of isolated CD45+ cells comprises from less than approximately 5 EU to less than approximately 0.5 EU of endotoxins per mL of formulation.
Embodiment 168. The method of any one of embodiments 89-167, wherein the HSPC, the Tmem, the Treg, or any combination thereof are obtained from an allogeneic HLA-matched donor relative to the human subject receiving the product.
Embodiment 169. The method of any one of embodiments 89-168, wherein the population of isolated CD45+ cells is from an allogeneic HLA-matched donor relative to the human subject receiving the product.
Embodiment 170. The method of any one of embodiments 89-167, wherein the HSPC, the Tmem, the Treg, or any combination thereof are obtained from an allogeneic donor having at least one HLA mismatch relative to the human subject receiving the product.
Embodiment 171. The method of any one of embodiments 89-167 or embodiment 170, wherein the population of isolated CD45+ cells is from an allogeneic donor having at least one HLA mismatch relative to the human subject receiving the product.
Embodiment 172. The method of embodiment 170 or embodiment 171, wherein the at least one HLA mismatch is at an allele selected from the group consisting of: HLA-A, HLA-B, HLA-C, HLA-DRB1, and any combination thereof.
Embodiment 173. The method of any one of embodiments 170-172, wherein the cells having at least one HLA mismatch are from a donor that is 6/8 HLA-mismatched relative to the human subject receiving the product or is 7/8 HLA-mismatched relative to the human subject receiving the product.
Embodiment 174. The method of embodiment 173, wherein the cells having at least one HLA mismatch are from a donor that is 7/8 HLA-mismatched relative to the human subject receiving the product.
Embodiment 175. The method of embodiment 174, wherein the donor that is 7/8 HLA-mismatched relative to the human subject receiving the product has a mismatch in HLA-A.
Embodiment 176. The method of embodiment 174, wherein the donor that is 7/8 HLA-mismatched relative to the human subject receiving the product has a mismatch in HLA-B.
Embodiment 177. The method of embodiment 174, wherein the donor that is 7/8 HLA-mismatched relative to the human subject receiving the product has a mismatch in HLA-C.
Embodiment 178. The method of embodiment 174, wherein the donor that is 7/8 HLA-mismatched relative to the human subject receiving the product has a mismatch in HLA-DRB1.
Embodiment 179. The method of any one of embodiments 170-178, wherein the donor that has the at least one HLA mismatch relative to the human subject receiving the product has a mismatched HLA allele as a result of the donor being homozygous for the HLA allele while the human subject receiving the product is heterogeneous for the HLA allele.
Embodiment 180. The method of any one of embodiments 170-178, wherein the donor that has at least one HLA mismatch relative to the human subject receiving the product has a mismatched HLA allele as a result of the donor being heterozygous for the HLA allele while the human subject receiving the product is homozygous for the HLA allele.
Embodiment 181. The method of any one of embodiments 170-178, wherein the donor that has at least one HLA mismatch relative to the human subject receiving the product has a mismatched HLA allele as a result of both the donor and the human subject receiving the product being heterozygous for the HLA allele.
Embodiment 182. The method of any one of embodiments 89-167, wherein the HSPC, the Tmem, the Treg, or any combination thereof are obtained from an allogeneic haploidentical donor relative to the human subject receiving the product.
Embodiment 183. The method of any one of embodiments 89-167 or embodiment 182, wherein the population of isolated CD45+ cells is from an allogeneic haploidentical donor relative to the human subject receiving the product.
Embodiment 184. The method of any one of embodiments 167-183, wherein the allogeneic donor is related to the human subject receiving the product.
Embodiment 185. The method of any one of embodiments 167-183, wherein the allogeneic donor is unrelated to the human subject receiving the product.
Embodiment 186. The method of any one of embodiments 89-185, wherein the method further comprises collecting one or more mobilized peripheral blood donations from the donor.
Embodiment 187. The method of embodiment 186, wherein the peripheral blood donations are mobilized by granulocyte colony-stimulating factor (G-CSF), granulocyte macrophage colony-stimulating factor (GM-CSF), plerixafor, or any combination thereof.
Embodiment 188. The method of embodiment 186 or embodiment 187, wherein at least one of the mobilized peripheral blood donations is processed and sorted using one or more immune-separation particles (ISPs) to enrich HSPC, Tmem, and Treg.
Embodiment 189. The method of embodiment 188, wherein the one or more ISPs comprise affinity reagents.
Embodiment 190. The method of embodiment 189, wherein the affinity reagents are immuno-magnetic separation particles.
Embodiment 191. The method of embodiment 190, wherein the immuno-magnetic separation particles are antibodies each conjugated to an iron-containing particle.
Embodiment 192. The method of any one of embodiments 189-191, wherein the affinity reagents comprise a plurality of CD34-reagents that binds to CD34 on an HSPC.
Embodiment 193. The method of any one of embodiments 188-192, wherein an average number of ISP's per HSPC in the population of isolated CD45+ cells is equal to or less than approximately 20,000.
Embodiment 194. The method of any one of embodiments 188-193, wherein an average number of ISP's per HSPC in the population of isolated CD45+ cells is from approximately 1000 to approximately 20,000.
Embodiment 195. The method of any one of embodiments 189-194, wherein the affinity reagents comprise a plurality of CD45RA-reagents that binds to one or more CD45RA receptors on a Tmem.
Embodiment 196. The method of any one of embodiments 189-195, wherein the affinity reagents comprise a plurality of CD25-reagents that binds to one or more CD25 receptors on a Treg.
Embodiment 197. The method of embodiment 196, wherein an average number of ISPs per Treg in the population of isolated CD45+ cells is equal or less than approximately 4000.
Embodiment 198. The method of embodiment 196 or embodiment 197, wherein an average number of ISPs per Treg in the population of isolated CD45+ cells is from approximately 1500 to approximately 2500.
Embodiment 199. The method of any one of embodiments 89-198, wherein the method further comprises administering a conditioning regimen prior to administration of the cellular therapy product. Embodiment 200. The method of embodiment 199, wherein the conditioning regimen is administered from approximately two days to approximately ten days before administration of the multi-component cellular therapy.
Embodiment 201. The method of embodiment 199 or embodiment 200, wherein the conditioning regimen is a total body irradiation-based (TBI-based) regimen or a total lymphatic irradiation-based (TLI-based) regimen.
Embodiment 202. The method of embodiment 201, wherein the TBI-based regimen comprises fractionated total body irradiation.
Embodiment 203. The method of embodiment 202, wherein the fractionated total body irradiation comprises 2 or more, 3 or more, 4 or more, 5 or more, 6 or more, 7 or more, 8 or more, 9 or more, 10 or more, 11 or more, 12 or more, 13 or more, 14 or more, or 15 or more doses.
Embodiment 204. The method of embodiment 201 or embodiment 202, wherein the fractionated total body irradiation comprises a total dose that ranges from approximately 500 to approximately 1600 cGy.
Embodiment 205. The method of any one of embodiments 201-204, wherein the TBI-based regimen further comprises one or more conditioning reagents.
Embodiment 206. The method of embodiment 205, wherein the one or more conditioning reagents are selected from the group consisting of: cyclophosphamide, etoposide, fludarabine, thiotepa, anti-thymocyte globulin (ATG), and any combination thereof.
Embodiment 207. The method of embodiment 199 or embodiment 200, wherein the conditioning regimen is a myeloablative conditioning regimen.
Embodiment 208. The method of embodiment 207, wherein the myeloablative conditioning regimen comprises one or more conditioning reagents.
Embodiment 209. The method of embodiment 208, wherein the one or more conditioning reagents are selected from the group consisting of: thiotepa, busulfan, melphalan, fludarabine, cyclophosphamide, anti-thymocyte globulin (ATG), and any combination thereof.
Embodiment 210. The method of embodiment 207, wherein the myeloablative conditioning regimen comprises three or more conditioning reagents, wherein at least one conditioning reagent comprises thiotepa.
Embodiment 211. The method of any one of embodiments 207-210, wherein the myeloablative conditioning regimen comprises one or more doses of busulfan, fludarabine, and thiotepa.
Embodiment 212. The method of embodiment 211, wherein the one or more doses of busulfan, fludarabine, and thiotepa comprise from approximately 5 to approximately 12 mg of thiotepa per kilogram of body weight of the human subject, from approximately 7 to approximately 11 mg of busulfan per kilogram of body weight of the human subject, and from approximately 100 to approximately 200 mg of fludarabine per meter2 body surface area respectively.
Embodiment 213. The method of any one of embodiments 89-212, wherein overall survival rate 12 months or more after administration of the cellular therapy product ranges from approximately 75% to approximately 100%.
Embodiment 214. The method of any one of embodiments 89-213, wherein overall survival of the human subject increases after administration of the cellular therapy product, relative to a corresponding human subject that has been administered a standard myeloablative allogeneic hematopoietic stem cell transplant (alloHSCT).
Embodiment 215. The method of any one of embodiments 89-214, wherein GVHD and relapse-free survival (GFRS) rate 12 months or more after administration of the cellular therapy product ranges from approximately 65% to approximately 100%.
Embodiment 216. The method of any one of embodiments 89-215, wherein GVHD and relapse-free survival (GFRS) of the human subject increases after administration of the cellular therapy product, relative to a corresponding human subject that has been administered a standard myeloablative allogeneic hematopoietic stem cell transplant (alloHSCT).
Embodiment 217. The method of any one of embodiments 89-216, wherein relapse-free survival rate 12 months or more after administration of the cellular therapy product ranges from approximately 70% to approximately 100%.
Embodiment 218. The method of any one of embodiments 89-217, wherein relapse-free survival of the human subject increases after administration of the cellular therapy product, relative to a corresponding human subject that has been administered a standard myeloablative allogeneic hematopoietic stem cell transplant (alloHSCT).
Embodiment 219. The method of any one of embodiments 89-218, wherein incidence of acute GVHD 100 or more days after administration of the cellular therapy product ranges from approximately 9% to approximately 31%.
Embodiment 220. The method of any one of embodiments 89-219, wherein incidence of chronic GVHD 100 or more days after administration of the cellular therapy product ranges from approximately 1% to approximately 10%.
Embodiment 221. The method of any one of embodiments 89-220, wherein incidence of post-transplant lymphoproliferative disorder (PTLD) 100 or more days after administration of the cellular therapy product ranges from approximately 1% to approximately 10%.
Embodiment 222. The method of any one of embodiments 89-221, wherein incidence of Grade 2 or higher infections 100 or more days after administration of the cellular therapy product ranges from approximately 23% to approximately 31%.
Embodiment 223. A method of treating a human subject having or suspected of having multiple sclerosis, the method comprising administering to the human subject a cellular therapy product comprising a population of isolated CD45+ cells, wherein the population of isolated CD45+ cells comprises an enriched population of CD34+ hematopoietic stem and progenitor cells (HSPC), an enriched population of CD45RA− memory T cells (Tmem), and an enriched population of fresh CD4+CD25+CD127dim regulatory T cells (Treg), and wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 224. A method of treating a human subject having or suspected of having multiple sclerosis, the method comprising administering to the human subject the cellular therapy product of any one of embodiments 1-84.
Embodiment 225. The method of embodiment 223 or embodiment 224, wherein the multiple sclerosis is primary progressive multiple sclerosis (PPMS).
Embodiment 226. The method of embodiment 225, wherein the PPMS has continued to progress.
Embodiment 227. The method of embodiment 226, wherein the PPMS has worsened by one or more points on the expanded disability status scale (EDSS).
Embodiment 228. The method of embodiment 223 or embodiment 224, wherein the multiple sclerosis is chronic progressive multiple sclerosis.
Embodiment 229. The method of embodiment 223 or embodiment 224, wherein the multiple sclerosis is secondary progressive multiple sclerosis.
Embodiment 230. The method of embodiment 223 or embodiment 224, wherein the multiple sclerosis is relapsing-remitting multiple sclerosis.
Embodiment 231. The method of embodiment 223 or embodiment 224, wherein the multiple sclerosis is acute relapsing multiple sclerosis.
Embodiment 232. The method of any one of embodiments 224-231, wherein acute GVHD-free survival of the human subject increases after administration of the cellular therapy product, relative to a corresponding human subject that has been administered a standard myeloablative allogeneic hematopoietic stem cell transplant (alloHSCT).
Embodiment 233. The method of any one of embodiments 224-232, wherein the human subject experiences acute GVHD-free survival 180 days or more after administration of the cellular therapy product.
Embodiment 234. The method of embodiment 232 or embodiment 233, wherein the human subject has not developed Grade 3 or higher acute GVHD.
Embodiment 235. The method of any one of embodiments 224-234, wherein event-free survival (EFS) of the human subject increases after administration of the cellular therapy product, relative to a corresponding human subject that has been administered a standard myeloablative allogeneic hematopoietic stem cell transplant (alloHSCT).
Embodiment 236. The method of any one of embodiments 224-235, wherein the human subject experiences EFS 6 months, 9 months, 12 months, 15 months, 18 months, 21 months, 24 months, 27 months, 30 months, 33 months, 36 months, 39 months, 42 months, 45 months, 48 months, 51 months, 54 months, 57 months, 60 months, or longer after administration of the cellular therapy product. Embodiment 237. The method of embodiment 235 or embodiment 236, wherein the human subject does not experience sustained loss of neurological function, does not experience relapse, does not develop 2 or more new and/or larger lesions, and/or does not experience death.
Embodiment 238. The method of embodiment 237, wherein loss of neurological functions is measured by Expanded Disability Status Scale (EDSS).
Embodiment 239. The method of embodiment 238, wherein an EDSS score of greater than 1 point indicates loss of neurological function.
Embodiment 240. The method of any one of embodiments 237-239, wherein the lesions are active.
Embodiment 241. The method of any one of embodiments 237-240, wherein the lesions are identified by magnetic resonance imaging (MRI).
Embodiment 242. The method of any one of embodiments 237-241, wherein the lesions are T1 lesions, T2 lesions, gadolinium-enhancing lesions, or any combination thereof, on brain MRI.
Embodiment 243. The method of any one of embodiments 237-242, wherein change in legion size is measured by changes in legion volume.
Embodiment 244. The method of any one of embodiments 89-243, wherein overall survival increases after administration of the cellular therapy product, relative to a corresponding human subject that has been administered a standard myeloablative allogeneic hematopoietic stem cell transplant (alloHSCT).
Embodiment 245. The method of any one of embodiments 89-244, wherein incidence of treatment related mortality (TRM) decreases after administration of the cellular therapy product, relative to a corresponding human subject that has been administered a standard myeloablative allogeneic hematopoietic stem cell transplant (alloHSCT).
Embodiment 246. The method of any one of embodiments 89-245, wherein disease activity is stable or decreases after administration of the cellular therapy product, relative to a corresponding human subject that has been administered a standard myeloablative allogeneic hematopoietic stem cell transplant (alloHSCT).
Embodiment 247. The method of any one of embodiments 89-246, wherein incidence of acute GVHD decreases after administration of the cellular therapy product, relative to a corresponding human subject that has been administered a standard myeloablative allogeneic hematopoietic stem cell transplant (alloHSCT).
Embodiment 248. The method of any one of embodiments 89-247, wherein severity of acute GVHD decreases or remains low after administration of the cellular therapy product, relative to a corresponding human subject that has been administered a standard myeloablative allogeneic hematopoietic stem cell transplant (alloHSCT).
Embodiment 249. The method of any one of embodiments 89-248, wherein incidence, severity, timing, or any combination thereof of Grade II to Grade IV acute GVHD decreases after administration of the cellular therapy product, relative to a corresponding human subject that has been administered a standard myeloablative allogeneic hematopoietic stem cell transplant (alloHSCT).
Embodiment 250. The method of any one of embodiments 89-249, wherein administration of the cellular therapy product prevents or reduces incidence of acute GVHD for 6 months, 9 months, 12 months, 15 months, 18 months, 21 months, 24 months, or longer.
Embodiment 251. The method of any one of embodiments 89-250, wherein incidence of chronic GVHD decreases after administration of the cellular therapy product, relative to a corresponding human subject that has been administered a standard myeloablative allogeneic hematopoietic stem cell transplant (alloHSCT).
Embodiment 252. The method of any one of embodiments 89-251, wherein incidence, severity, timing, or any combination thereof of moderate to severe chronic GVHD decreases after administration of the cellular therapy product, relative to a corresponding human subject that has been administered a standard myeloablative allogeneic hematopoietic stem cell transplant (alloHSCT) Embodiment 253. The method of any one of embodiments 89-252, wherein administration of the cellular therapy product prevents or reduces incidence of chronic GVHD for 6 months, 9 months, 12 months, 15 months, 18 months, 21 months, 24 months, or longer.
Embodiment 254. The method of any one of embodiments 89-253, wherein incidence of secondary graft failure decreases after administration of the cellular therapy product, relative to a corresponding human subject that has been administered a standard myeloablative allogeneic hematopoietic stem cell transplant (alloHSCT).
Embodiment 255. The method of any one of embodiments 89-254, wherein incidence of Grade 3 or higher infectious disease complications decreases after administration of the cellular therapy product, relative to a corresponding human subject that has been administered a standard myeloablative allogeneic hematopoietic stem cell transplant (alloHSCT).
Embodiment 256. The method of any one of embodiments 224-255, wherein administration of the cellular therapy product modulates one or more multiple sclerosis biomarkers.
Embodiment 257. The method of embodiment 256, wherein the biomarker is oligoclonal bands protein level in level in cerebrospinal fluid (CSF) Embodiment 258. The method of embodiment 257, wherein administration of the cellular therapy product decreases the oligoclonal bands protein level in the CSF of the human subject.
Embodiment 259. The method of embodiment 256, wherein the biomarker is neurofilament protein level in cerebrospinal fluid (CSF).
Embodiment 260. The method of embodiment 259, wherein administration of the cellular therapy product decreases or stabilized the neurofilament protein level in the CSF of the human subject.
Embodiment 261. The method of any one of embodiments 224-260, wherein administration of the cellular therapy product improves quality of life of the human subject.
Embodiment 262. The method of embodiment 261, wherein the quality of life is measured by Multiple Sclerosis Quality of Life-54 (MSQOL-54), Short From-36 (SF36), Multiple Sclerosis Impact Scale-29 (MSIS-29), Patient Determined Disease Steps (PDDS), or any combination thereof.
Embodiment 263. The method of any one of embodiments 224-262, wherein administration of the cellular therapy product improves one or more radiographic markers of primary progressive multiple sclerosis (PPMS).
Embodiment 264. The method of embodiment 263, wherein the one or more markers of PPMS comprise number of legions on brain MRI, legion volume, cortical gray matter volume, thalamic volume, whole brain volume, or any combination thereof.
Embodiment 265. The method of embodiment 264, wherein the legions are T1 lesions, T2 lesions, gadolinium-enhancing lesions, or any combination thereof.
Embodiment 266. The method of any one of embodiments 89-265, wherein the human subject is approximately 3 months of age or older.
Embodiment 267. The method of any one of embodiments 89-266, wherein the human subject is from between approximately 3 months to approximately 18 years of age.
Embodiment 268. The method of any one of embodiments 89-232, wherein the human subject is approximately 12 years of age or older.
Embodiment 269. The method of any one of embodiments 89-266 or embodiment 268, wherein the human subject is between approximately 12 years to approximately 65 years of age.
Embodiment 270. The method of any one of embodiments 89-266 or embodiment 268, wherein the human subject is between approximately 18 years to approximately 65 years of age.
Embodiment 271. The method of any one of embodiments 89-266 or embodiment 268, wherein the human subject is between approximately 12 years to approximately 75 years of age.
Embodiment 272. The method of embodiment 271, wherein the human subject is from approximately 65 years of age to approximately 75 years of age.
Embodiment 273. The method of any one of embodiments 89-266 or embodiment 268, wherein the human subject is from between approximately 3 months to approximately 75 years of age.
Embodiment 274. The method of any one of embodiments 89-273, wherein the human subject has received from one to five previous lines of therapy.
Embodiment 275. A cellular therapy kit comprising the cellular therapy product of any one of embodiments 1-88.
Embodiment 276. A cellular therapy kit comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA⁻ memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg),
   wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 277. A cellular therapy kit comprising a container comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 278. The cellular therapy kit of any one of embodiments 275-277, wherein the kit further comprises written instructions for using the cellular therapy for treating a hematologic malignancy in a human subject.
Embodiment 279. The cellular therapy kit of embodiment 278, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).
Embodiment 280. The cellular therapy kit of any one of embodiments 275-279, wherein the kit further comprises written instructions for using the cellular therapy for treating multiple sclerosis in a human subject.
Embodiment 281. A unit dose comprising the cellular therapy product of any one of embodiments 1-88.
Embodiment 282. A unit dose of comprising a population of isolated CD45+ cells, wherein the population of isolated CD45+ cells comprises:
a) approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA⁻ memory T cells (Tmem); and/or
c) approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), and
   wherein the population of isolated CD45+ cells has been depleted of naïve conventional CD3+CD25⁻CD45RA+ T cells (Tcon) to comprise less than approximately 5.0×10⁵ naïve conventional CD3+CD25⁻CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 283. The unit dose of embodiment 282, wherein the population of isolated CD45+ cells further comprises one or more excipients.
Embodiment 284. A unit dose comprising:
a) approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC), and one or more excipients;
b) approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem), and one or more excipients; and
c) approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), and one or more excipients, wherein the unit dose has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).
Embodiment 285. An article of manufacture comprising the cellular therapy product of any one of embodiments 1-88.
Embodiment 286. An article of manufacture comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 287. An article of manufacture comprising a container comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg),
wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 288. The article of manufacture of any one of embodiments 285-287, further comprising instructions for administering the article of manufacture to a human subject treat a hematologic malignancy.
Embodiment 289. The article of manufacture of embodiment 288, wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).
Embodiment 290. The article of manufacture of any one of embodiments 285-289, further comprising instructions for administering the article of manufacture to a human subject treat multiple sclerosis.
Embodiment 291. A container comprising the cellular therapy product of any one of embodiments 1-88.
Embodiment 292. A container comprising a population of isolated CD45+ cells, wherein the population of isolated CD45+ cells comprises:
a) a dose of approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC);
b) a dose of approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA⁻ memory T cells (Tmem); and
c) a dose of approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), wherein the population of isolated CD45+ cells has been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon), and wherein the population of isolated CD45+ cells is formulated with an excipient at a neutral pH.
Embodiment 293. A container comprising:
a) approximately 1.0×10⁵ to approximately 1.0×10⁸ CD34+ hematopoietic stem and progenitor cells (HSPC) per kilogram of body weight of a human subject receiving the product or a total dose of approximately 5.0×10⁵ to approximately 1.5×10¹⁰ CD34+ hematopoietic stem and progenitor cells (HSPC), and one or more excipients;
b) approximately 1.0×10⁵ to approximately 1.0×10⁸ CD45RA− memory T cells (Tmem) per kilogram of body weight of the human subject receiving the product or a total dose of approximately 5.0×10⁵ to approximately 1.5×10¹⁰ CD45RA− memory T cells (Tmem), and one or more excipients; and
c) approximately 1.0×10⁵ to approximately 2.0×10⁷ fresh CD4+CD25+CD127dim regulatory T cells (Treg) per kilogram of body weight of the human subject receiving the product or a total dose of approximately 5.0×10⁵ to approximately 3.0×10⁹ isolated fresh +CD4+CD25+CD127dim regulatory T cells (Treg), and one or more excipients, wherein the HSPC, Tmem, and/or Treg have been depleted of naïve conventional CD3+CD25−CD45RA+ T cells (Tcon) to comprise less than approximately 5.0×10⁵ naïve conventional CD3+CD25−CD45RA+ T cells (Tcon) per kilogram of body weight of the human subject receiving the product, less than approximately 6.0×10⁷ total naïve conventional CD3+CD25−CD45RA+ T cells (Tcon), or less than approximately 1.0×10⁶ total naïve conventional CD3+CD25−CD45RA+ T cells (Tcon).

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Clinical Study

### Study Design

A multicenter, open-label dose escalation and expansion Phase 1 study was conducted in adult subjects undergoing myeloablative allogeneic hematopoietic cell transplantation (MA-alloHCT).

Primary endpoints of the study include the incidence of dose-limiting toxicities (DLTs) and the incidence of primary graft failure.

Secondary endpoints for all arms include neutrophil engraftment, platelet engraftment, incidence of secondary graft failure, incidence and severity of treatment-emergent adverse events (TEAEs), incidence and severity of acute GVHD, for example, grade 3-4 acute GVHD, incidence and severity of steroid-refractory acute GVHD, for example, grade 3-4 steroid-refractory acute GVHD, incidence and severity of chronic GVHD, incidence of post-transplant lymphoproliferative disorder (PTLD), non-relapse mortality (NRM), disease relapse, disease-free survival (DFS), GVHD and relapse free survival (GRFS), and overall survival.

### Dose Escalation

For each arm, dose escalation followed a 3+3 dose escalation approach for the Tmem component, and proceeded until a single maximum tolerated dose (MTD) was determined. Table 7A illustrates the subject cohorts for dose escalation in Arm A and Arm C. Table 7B illustrates the subject cohorts for dose escalation in Arm B. Doses of all cell types were based on bodyweight of the recipient.

TABLE 7A
Dose escalation cohorts for Arm A and Arm C.

| Cohort | Tmem | CD34+ HSPC | Treg |
|---|---|---|---|
| 1 | 3 × 10⁶/kg | Min: 2.0 × | Min: 0.5 × |
| 2 | 10 × 10⁶/kg | 10⁶/kg | 10⁶/kg |
| 3 | 30 × 10⁶/kg | Target: 10 × | Target: 3.5 × |
| 4 | 100 × 10⁶/kg | 10⁶/kg | 10⁶/kg |

TABLE 7B
Dose escalation cohorts for Arm B.

| Cohort | Tmem | CD34+ HSPC | Treg |
|---|---|---|---|
| 3 | 10 × 10⁶/kg | Min: 5.0 × | Min: 0.5 × |
| 4 | 30 × 10⁶/kg | 10⁶/kg | 10⁶/kg |
| 5 | 100 × 10⁶/kg | Target: 10.0 × | Target: 3.5 × |
| | | 10⁶/kg | 10⁶/kg |

The first subject in each cohort of each arm was observed for the entire DLT Evaluation Period before enrollment of the remaining subjects in the cohort (i.e., the Day 0 dates of the first and second patients in each escalation cohort should be separated by 28 days). Each cohort initially enrolled 3 subjects. After all 3 patients in a particular cohort were evaluated for toxicity for the entire DLT Evaluation Period, then:

If no patients receiving the cellular therapy product of the present disclosure encountered a DLT, escalation continued to the next higher dose level.

If 1 of 3 patients receiving the cellular therapy product of the present disclosure encountered a DLT, an additional 3 patients were enrolled at that dose level. After all 6 patients in that particular cohort were evaluated for toxicity for the entire DLT Evaluation Period, if no more than 1 of 6 patients receiving the cellular therapy product of the present disclosure encountered a DLT, escalation continued to the next higher dose level.

If ≥2 patients receiving the cellular therapy product of the present disclosure encountered a DLT, further accrual at that dose level or higher ceased, and the prior dose level was declared the MTD.

If no cohorts ceased due to DLT, the highest dose level completed was declared the MTD.

Enrollment to Arm C.1 cohorts was only initiated following the clearance of the Arm A.1 cohort with the corresponding Tmem dose.

### Recipient Population

Subjects were eligible to receive a composition of the disclosure if they met all the following Inclusion criteria:
Age ≥12 and ≤65 years at the time of enrollment

Patients with the following histopathologically-confirmed diseases were eligible:

Acute myeloid, lymphoid or mixed phenotype leukemia in complete remission (CR) or CR with incomplete hematologic recovery (CRi) as defined by:
CR: all of the following: Bone marrow blasts <5%; Absence of circulating blasts and blasts with Auer rods; Absence of extramedullary disease; ANC≥1.0×10⁹/L (1,000/μL); Platelet count ≥100×10⁹/L (100,000/μL); Independence of red cell transfusions
CRi: All CR criteria except for residual neutropenia (<1.0×10⁹/L) or thrombocytopenia (<100×10⁹/L)
Acute myeloid, lymphoid or mixed phenotype leukemia not in morphologic CR with bone marrow infiltration by leukemic blasts of ≤10%. Patients with active leukemia as described in this criteria may only be consented following approval by the medical monitor.

Chronic myeloid leukemia (CML) that is: 1) In myeloid or lymphoid blast crisis or accelerated phase provided that patients are in CR or CRi with regard to the blast crisis. 2) Chronic phase CML resistant to or intolerant of multiple- first and second-generation-tyrosine kinase inhibitors.

High or very high risk myelodysplastic syndrome (MDS) including both of the following: 1) Revised International Prognostic Scoring System (IPSS-R) score >4.5. 2) Bone marrow blasts <10% at screening

Myelofibrosis (MF) that is eligible for transplant per National Comprehensive Cancer Network Guidelines. Specifically, patients should be diagnosed with MF that is either: 1) intermediate-2- or high-risk according to the IPSS, DIPSS or DIPSS-plus scoring systems, or 2) intermediate-1-risk disease associated with high risk features such as high symptoms burden, low platelet counts, or complex cytogenetics; per NCCN guidelines and/or Investigator judgement.

Planning to undergo myeloablative allogeneic hematopoietic cell transplantation (MA-alloHCT) including a suitable myeloablative conditioning regimen.

Matched to a related or unrelated donor as follows: (all HLA match were typed using DNA-based high resolution methods).

Arm A: Either one of: i) Matched related donor who is 8/8 allele matched at HLA-A, -B, -C, and -DRB1 (MRD), or ii) Matched unrelated donor who is 8/8 allele matched at HLA-A, -B, -C, and -DRB1 (MUD) or who is 7/8 allele matched at HLA-A, -B, -C, or -DRB1 (MMUD)

Arm B: haploidentical related donor who is a ≥4/8 but <7/8 allele match at HLA-A, -B, -C, and -DRB1, with at most one mismatch per locus.

Arm C: MRD or MUD who is an 8/8 allele match for HLA-A, -B, -C, and -DRB1.

Estimated glomerular filtration rate (eGFR)>50 mL/minute; or >30 mL/minute for patients enrolled onto Arm C.

Cardiac ejection fraction at rest ≥45% or shortening fraction of ≥27% by echocardiogram or radionuclide scan (MUGA).

Diffusing capacity of the lung for carbon monoxide (DLCO) (adjusted for hemoglobin) ≥50%

Total bilirubin <1.5 times upper limit of normal (ULN) (<3 times if attributed to Gilbert's syndrome) and ALT/AST<3 times ULN.

Recipients in screening screen negative for SARS-CoV-2 RNA using a PCR-based test prior to enrollment as follows: a) The SARS-CoV-2 testing screening test should be timed such that the results are available prior to the start of the recipient's conditioning regimen. Efforts should be made to minimize the window of time between test result availability and the start of conditioning. b) Recipients in screening who test positive for SARS-CoV-2 were ineligible but may be considered eligible for future trial participation provided that they are cleared for transplantation per the most current ASTCT guidelines or as per institutional guidelines. c) An exception may be made with Medical Monitor approval on a case-by-case basis while weighing the risks of delaying or altering therapy for the underlying disease.

Subjects were ineligible to receive the composition of the disclosure if they met any of the following exclusion criteria:
Prior allogeneic HCT

Currently receiving corticosteroids or other immunosuppressive therapy. Topical corticosteroids or oral systemic corticosteroid doses less than or equal to 10 mg/day were allowed.
Planned donor lymphocyte infusion (DLI)

Planned pharmaceutical in vivo or ex vivo T cell depletion, e.g., post-transplant cyclophosphamide (Cy) or alemtuzumab. Anti-thymocyte globulin was allowed as described in the Allowed MAC Regimens section.

Positive anti-donor HLA antibodies against a mismatched allele in the selected donor determined by either: a) a positive crossmatch test of any titer (by complement-dependent cytotoxicity or flow cytometric testing), or b) the presence of anti-donor HLA antibody to any of the following HLA loci: HLA-A, -B, -C, -DRB1, -DQB1, -DQA1, -DPB1, or -DPA1, with mean fluorescence intensity (MFI)>1000 by solid phase immunoassay.
Karnofsky or Lansky performance score <70%
Hematopoietic cell transplantation-specific Comorbidity Index (HCT-CI)>4 (for adult patients)

Uncontrolled bacterial, viral or fungal infections (currently taking antimicrobial therapy and with progression or no clinical improvement) at time of enrollment.
Seropositive for HIV-1 or -2, HTLV-1 or -2

Known allergy or hypersensitivity to, or intolerance of, tacrolimus.

Documented allergy or hypersensitivity to iron dextran or bovine, murine, algal or Streptomyces avidinii proteins

Any uncontrolled autoimmune disease requiring active immunosuppressive treatment.

Concurrent malignancies or active disease within 1 year, except nonmelanoma skin cancers that have been curatively resected.
History of idiopathic or secondary myelofibrosis
Women who are pregnant or breastfeeding

Women of childbearing potential (WOCBP) or men who have sexual contact with WOCBP unwilling to use effective forms of birth control or abstinence for one year after transplantation.

Any serious medical condition or abnormality in clinical laboratory tests that, in the Investigator's or Medical Monitor's judgment, precludes the Recipient's safe participation in and completion of the study, or which could affect compliance with the protocol or interpretation of results.

Table 8A and Table 8B show the demographics, primary disease, disease status, conditioning regiment, and median follow up range for a representative subset of the subjects. Abbreviations: AML, acute myeloid leukemia; ALL, acute lymphocytic leukemia; CML, chronic myeloid leukemia MDS, myelodysplastic syndrome; MF, myelofibrosis; CR MRD-, complete remission minimal residual disease negative; CR MRD+, complete remission minimal residual disease positive; Cy, cyclophosphamide; VP-16, etoposide; Bu, busulfan; flu, fludarabine; TBI, total body irradiation; Thio, thiotepa; TFT, TBI/flu/thiotepa; BFT, busulfan/flu/thiotepa. MRD, 8/8 matched related donor; MSD, 8/8 matched sibling donor; MUD, 8/8 matched unrelated donor; MMUD, 7/8 mismatched unrelated donor; Haplo, haploidentical;

TABLE 8A
Representative Subset of Subjects.

| | N = 68 |
|---|---|
| Arms | |
| Arm A: MRD | 20 |
| Arm A: MUD | 6 |
| Arm A: MMUD | 15 |
| Arm B: Haplo | 23 |
| Arm C: MRD/MUD, no GVHD | 4 |
| Median age (range) | 45 vears |
| | (24-65) |
| M:F | 41:27 |
| Race | |
| White | 27 |
| Latino | 17 |
| African American | 8 |
| Asian | 11 |
| Other | 5 |
| Disease | |
| AML | 37 |
| ALL | 20 |
| MF | 1 |
| CML | 6 |
| MDS | 4 |
| Conditioning regimen | |
| Bu/Cy | 5 |
| TBI/VP or Flu or Cy or Cy/Thio | 15 |
| TFT | 16 |
| BFT | 28 |
| Bu/Flu | 4 |
| Median follow up (range) | 222 days |
| | (10-1020) |

TABLE 8B
Representative Subset of Subjects Separated by Different Arms of the Study.

| | ARMS | | | |
|---|---|---|---|---|
| | Arm A + C | | Arm B | |
| | Total NO. of Subjects | | | |
| | 23 (with >30 | 23 (22 Tmem | 22 (dose | 21(with >30 |
| | days follow up) | dose >~10 × 10⁶) | expansion) | days follow up) |
| Median age (range) | 44 years | 43 years | 44 years | 44 years |
| | (26-65) | (21-63) | (21-63) | (21-63) |
| M:F | 143 | 143 | 1m3 | 125 |
| Race | | | | |
| White | 12 | 7 | 6 | 5 |
| Latino | 6 | 6 | 6 | 5 |
| African American | 2 | 4 | 4 | 4 |
| Asian | 3 | 4 | 4 | 4 |
| Other | — | 2 | 2 | 2 |
| Disease | | | | |
| AML | 10 | 12 | 12 | 11 |
| ALL | 7 | 8 | 8 | 8 |
| CML | 4 | 2 | 2 | 2 |
| MDS | 1 | 1 | — | — |
| MF | 1 | | | |
| Disease Status | | | | |
| CR MRD− | 22 | 20 | 19 | 19 |
| CR MRD+ | 1 | 3 | 3 | 2 |
| Conditioning regimen | | | | |
| Bu/Cy or Bu/Flu | 2 | 1 | — | — |
| TBI/VP-16 or Flu or Cy or | 11 | 2 | 2 | 2 |
| Cy/Thio | | | | |
| TFT | 1 | 11 | 11 | 11 |
| BFT | 9 | 9 | 9 | 8 |
| Median follow up (range) | 300 days | 262 days | 297 (10- | 333 (39- |
| | (30-1011) | (10-1019) | 1019) | 1019) |

### Recipient Assessment

Subjects enrolled in the study were clinically assessed prior to and after the administration of the pharmaceutical treatments provided herein, which comprises a cell population comprising CD34+ HSPC, which is enriched in Treg, iNKT and Tmem, and is depleted of naïve conventional αβ-T cells. TABLE 9 (A-C) is a Schedule of Activity of the recipients, which shows the types of clinical assessments performed, and the schedule of these assessments.

TABLE 9A
Recipient Schedule of Activity

| | Screening¹ | ATG¹⁹ | MAC¹ | HCT | | | Follow-Up | | |
|---|---|---|---|---|---|---|---|---|---|
| | Day (vs. first HCT infusion) | | | | | | | | |
| | | | | | | | +7 | +14 | +21 |
| | −40 | −10 | −10 | | | | Window | | |
| | to −10 | to −8 | to −2 | −1 | 0 | +1 | ±1 d | ±2 d | ±2 d |
| Informed Consent | X | | | | | | | | |
| Inclusion/Exclusion Criteria | X | | | | | | | | |
| Demographics | X | | | | | | | | |
| Medical and Disease History | X | | | | | | | | |
| Prior Therapies | X | | | | | | | | |
| Physical Examination | X | | | | X | X | X | X | X |
| Vital Signs³ | X | | | | X⁴ | X | X | | |
| Karnofsky or Lansky Score | X | | | | X | | | | |
| HCT-CI index | X | | | | | | | | |
| CBC with differential⁵ | X | | | | At least QD until neutrophil engraftment, then 2x/ | | | | |
| | | | | | wk to D + 56, then Q2W to D + 100 | | | | |
| Clinical Chemistry⁵ | X | | | | At least weekly to D + 56, then Q2W to D + 100 | | | | |
| Coagulation⁵ | X | | | | | | | | |
| Fenitin | X | | | | | | | | |
| C-reactive protein | X | | | | | | | | |
| Serum Ig levels | X | | | | | | | | |
| (IgA, IgG, IgM) | | | | | | | | | |
| T, B, and NK cell counts¹⁸ | | | | | | | | | |
| Recipient Infectious | X | | | | As clinically indicated | | | | |
| Disease Markers⁵ | | | | | | | | | |
| PCR-based testing for | X | | | | | | | | |
| SARS-CoV-2¹⁶ | | | | | | | | | |
| Urinanalysis⁵ | | | | | | | | | |

| | Follow-Up | | | | | | | | |
|---|---|---|---|---|---|---|---|---|---|
| | Day (vs. first HCT infusion) | | | | | | | | |
| | +28 | +35 | +42 | +49 | +56 | +100 | +180 | +365 | |
| | Window | | | | | | | | |
| | ±2 d | ±3 d | ±3 d | ±3 d | ±3 d | ±7 d | ±14 d | ±21 d | LTFU² |
| Informed Consent | | | | | | | | | |
| Inclusion/Exclusion Criteria | | | | | | | | | |
| Demographics | | | | | | | | | |
| Medical and Disease History | | | | | | | | | |
| Prior Therapies | | | | | | | | | |
| Physical Examination | X | X | X | X | X | X | X | X | |
| Vital Signs³ | X | X | X | X | X | X | X | X | |
| Karnofsky or Lansky Score | X | | | | X | X | X | X | |
| HCT-CI index | | | | | | | | | |
| CBC with differential⁵ | At least QD until neutrophil engraftment, then 2x/ | | | | | | | | |
| | wk to D + 56, then Q2W to D + 100 | | | | | | | | |
| Clinical Chemistry⁵ | At least weekly to D + 56, then Q2W to D + 100 | | | | | | | | |
| Coagulation⁵ | | | | | | | | | |
| Fenitin | | | | | | | | | |
| C-reactive protein | | | | | | | | | |
| Serum Ig levels | X | | | | X | X | X | X | |
| (IgA, IgG, IgM) | | | | | | | | | |
| T, B, and NK cell counts¹⁸ | X | | | | X | X | X | X | |
| Recipient Infectious | As clinically indicated | | | | | | | | |
| Disease Markers⁵ | | | | | | | | | |
| PCR-based testing for | | | | | | | | | |
| SARS-CoV-2¹⁶ | | | | | | | | | |
| Urinanalysis⁵ | | | | | | | | | |

TABLE 9B
Recipient Schedule of Activity

| | Screening¹ | ATG¹⁹ | MAC¹ | HCT | | | Follow-Up | |
|---|---|---|---|---|---|---|---|---|
| | Day (vs. first HCT infusion) | | | | | | | |
| | | | | | | | +7 | +14 |
| | −40 | −10 | −10 | | | | Window | |
| | to −10 | to −8 | to −2 | −1 | 0 | +1 | ±1 d | ±2 d |
| Serum or urine β-HCG | X | | | | | | | |
| (if WOCBP⁶) | | | | | | | | |
| Electrocardiogram, 12-lead⁷ | X | | | | | | | |
| DLCO Pulmonary Function | X | | | | | | | |
| Test⁸ | | | | | | | | |
| LVEF⁹ | X | | | | | | | |
| ABO and Rh Typing¹⁵ | X | | | | | | | |
| HLA Typing and anti-donor | X | | | | | | | |
| HLA antibody testing⁵, ¹⁵ | | | | | | | | |
| Disease Evaluation¹⁰ | X | | | | | | | |
| includes central | | | | | | | | |
| laboratory analysis | | | | | | | | |
| Lineage-specific Donor | X | | | | | | | |
| Chimerism | | | | | | | | |
| Survival Status | | | | | | | | |
| Conditioning Regimen¹¹ | | | X | | | | | |
| Anti-thymocyte globulin¹⁷ | | X | | | | | | |
| Orca-Q Prime (formerly | | | | | X | | | |
| OrcaGraft Prime) | | | | | | | | |
| Orca-Q Supplement (formerly | | | | | X¹² | | | |
| OrcaGraft Supplement) | | | | | | | | |
| GVHD Prophylaxis | | | | Arms A and B only: Beginning on Day -1 | | | | |
| EBV/CMV NAT | | | | | | | Weekly from Day +7 through Day +56 | |
| GVHD Assessment | | | | | | | X | X |
| Adverse Events | | | | | | | | |
| Concomitant Therapies | | | | | | | | |
| Central Laboratory Tests | | | | | | | | |

| | Follow-Up | | | | | | | | | |
|---|---|---|---|---|---|---|---|---|---|---|
| | Day (vs. first HCT infusion) | | | | | | | | | |
| | +21 | +28 | +35 | +42 | +49 | +56 | +100 | +180 | +365 | |
| | Window | | | | | | | | | |
| | ±2 d | ±2 d | ±3 d | ±3 d | ±3 d | ±3 d | ±7 d | ±14 d | ±21 d | LTFU² |
| Serum or urine β-HCG | | | | | | | | | | |
| (if WOCBP⁶) | | | | | | | | | | |
| Electrocardiogram, 12-lead⁷ | | | | | | | | | | |
| DLCO Pulmonary Function | | | | | | | | | | |
| Test⁸ | | | | | | | | | | |
| LVEF⁹ | | | | | | | | | | |
| ABO and Rh Typing¹⁵ | | | | | | | | | | |
| HLA Typing and anti-donor | | | | | | | | | | |
| HLA antibody testing⁵, ¹⁵ | | | | | | | | | | |
| Disease Evaluation¹⁰ | | | | | | | X | | X | X |
| includes central | | | | | | | | | | |
| laboratory analysis | | | | | | | | | | |
| Lineage-specific Donor | | X | | | | X | X | X | X | |
| Chimerism | | | | | | | | | | |
| Survival Status | | X | | | | X | X | X | X | X |
| Conditioning Regimen¹¹ | | | | | | | | | | |
| Anti-thymocyte globulin¹⁷ | | | | | | | | | | |
| Orca-Q Prime (formerly | | | | | | | | | | |
| OrcaGraft Prime) | | | | | | | | | | |
| Orca-Q Supplement (formerly | | | | | | | | | | |
| OrcaGraft Supplement) | | | | | | | | | | |
| GVHD Prophylaxis | Arms A and B only: Beginning on Day -1 | | | | | | | | | |
| EBV/CMV NAT | Weekly from Day +7 through Day +56 | | | | | | X | X | X | |
| GVHD Assessment | X | X | X | X | X | X | X | X | X | |
| Adverse Events | | | | | | | | | | |
| Concomitant Therapies | | | | | | | | | | |
| Central Laboratory Tests | | | | | | | | | | |

TABLE 9C
Recipient Schedule of Activity

| | Screening¹ | ATG¹⁹ | MAC¹ | HCT | | | Follow-Up | | | | | | | | | | | |
|---|---|---|---|---|---|---|---|---|---|---|---|---|---|---|---|---|---|---|
| | Day (vs. first HCT infusion) | | | | | | | | | | | | | | | | | |
| | | | | | | | +7 | +14 | +21 | +28 | +35 | +42 | +49 | +56 | +100 | +180 | +365 | |
| | −40 | −10 | −10 | | | | Window | | | | | | | | | | | |
| | to −10 | to −8 | to −2 | −1 | 0 | +1 | ±1 d | ±2 d | ±2 d | ±2 d | ±3 d | ±3 d | ±3 d | ±3 d | ±7 d | ±14 d | ±21 d | LTFU² |
| Blood for | X | | | X | | | | X | | X | | | | X | X | X | X | |
| biomarker | | | | | | | | | | | | | | | | | | |
| analysis¹³ | | | | | | | | | | | | | | | | | | |
| Blood for | | | | | Once at onset of GVHD prior to treatment | | | | | | | | | | | | | |
| GVHD | | | | | | | | | | | | | | | | | | |
| biomarkers¹⁴ | | | | | | | | | | | | | | | | | | |

1. Myeloablative conditioning is described in the Treatment Section. The specific regimen, including duration, was determined for each recipient per Investigator's choice and local institutional standards. As a result, the specific days of administration varied from those shown in the schedule. The screening window extended from Day −40 through the start of the conditioning regiment. Therefore, depending on the conditioning regimen chosen, the screening window may not have ended and the MAC window may not have begun exactly on Day −10 as depicted above.
2. Long-term follow-up occured every 6 months until death, loss to follow-up, withdrawal of consent, or study termination.
3. Vital signs included temperature, pulse, supine blood pressure, respiratory rate, pulse oximetry, and weight
4. Vital signs, weight excluded, were monitored every 30±5 min for 4 hours after the start of cell infusions
5. Specific tests common to the field and are known to ones that are skilled in the art.
6. Woman of childbearing potential, as defined herein.
7. Electrocardiogram (ECG), 12-lead, was performed supine or sitting, at rest.
8. Diffusing capacity of the lung for carbon monoxide (DLCO), adjusted for hemoglobin, was performed <42 days prior to initiation of conditioning
9. Left ventricular ejection fraction (LVEF) was estimated by echocardiogram or multigated acquisition (MUGA) scan, performed up to <42 days prior to initiation of conditioning
10. Disease Evaluation included at least a bone marrow aspirate/biopsy as well as other tests, such as cytogenetic, as clinically indicated. A part of the sample was collected for central laboratory MRD assessment and biomarker analysis. Included collection of data from tests obtained incidentally during usual standard care at non-protocol-specified timepoints. In LTFU, formal disease assessment was performed according to standard care, and dates of disease recurrence or progression, subsequent anticancer therapies, dates and responses, were recorded where applicable and available. Central laboratory MRD assessment was not performed during LTFU. See the laboratory manual for instructions for the collection of central labs.
11. Allowed conditioning regimens are described in Treatment Section.
12. Cellular therapy product supplement was administered in subjects for whom the primary cellular therapy product of the present disclosure did not provide sufficient target numbers of HSPC and or Tmem, as soon as available after administration of the primary product, e.g., on Day 0 or Day +1.
13. Included, but is not limited to, percentage and absolute count of by flow cytometry of CD4+ and CD8+ T cells, Treg, iNKT, NK cells, B cells, monocytes, and granulocytes as well as quantification of T cell receptor excision circles (TREC)
14. GVHD biomarkers included a blood sample archived for possible future analysis for biomarkers of GVHD, such as ST2 or Reg3a. If a patient developed both aGVHD and cGVHD, a sample was drawn at the onset of both aGVHD and cGVHD; i.e., if a patient with a history of aGVHD subsequently developed cGVHD, an additional biomarker sample was drawn.
15. HLA typing procedures and ABO/Rh testing performed prior to the screening period were used for screening.
16. See Inclusion criteria #9.
17. Patients with haploidentical or single-HLA allele mismatched (7/8) donors who underwentconditioning with the BFT or TBI/Flu regimens also received ATG as described in the Treatment Section.
18. T, B, and NK cell counts were performed on the peripheral blood and included assessment of B cells, NK cells, CD4+ T cells, and CD8+ T cells.
When deemed clinically appropriate by the Investigator, deviations from protocol-specified follow-up clinic visits were allowed to minimize the participant's risk of exposure to SARS-CoV-2. This may have included conversion of in-person visits to alternative visits (e.g., phone contacts or video visits).

### Donor Population

Suitable HLA-matched, 1 antigen/allele mismatched, or haploidentical donors were identified and included based on the following inclusion criteria:
Age ≥16 and ≤75 years at time of enrollment

Match to the patient as follows:
Arm A: Either one of:
   a. Matched related donor who is an 8/8 match for HLA-A, -B, -C, and -DRB1, all typed using DNA-based high resolution methods; or
   b. Matched unrelated donor who is a 7 or 8/8 match at HLA-A, -B, -C, and -DRB1, all typed using DNA-based high resolution methods.
Arm B: haploidentical related donor who is a ≥4/8 but <7/8 match at HLA-A, -B, -C, and -DRB1 (typed using DNA-based high resolution methods), with at most one mismatch per locus.
Arm C: Matched related or unrelated donor who is an 8/8 match for HLA-A, -B, -C, and -DRB1, all typed using DNA-based high resolution methods.

Willing to donate PBSC for up to two consecutive days.

Donors meeting any of the following exclusion criteria were not eligible:
Evidence of active infection
Seropositive for HIV-1 or -2, HTLV-1 or -2
Positive for active or chronic Hepatitis B (HBV) based on serology [HBV surface antigen (HBsAg), total anti-Hepatitis B core antibody (HBcAb, IgG and IgM), and Hepatitis B surface antibody (HBsAb)] or by nucleic acid testing (NAT) testing. Donors with ambiguous HBV serology results should be tested for HBV by NAT. Error!Hyperlink reference not valid.
Positive for anti-hepatitis C (HCV) antibody or HCV NAT.

Potential for Zika virus infection as defined as any of the following:
a. Medical diagnosis of Zika virus infection in the past 6 months
b. Residence in, or travel to, an area with active Zika virus transmission within the past 6 months.
c. Sex within the past 6 months with a person who is known to have either of the risk factors listed above (donor exclusion criterion 5.a or 5.b)

Donors determined to be ineligible based on the results of Zika virus screening may have been determined to be eligible if: The donor had no signs or symptoms consistent with active Zika virus infection and either of the following were true:
a. The donor was a first-degree or second-degree blood relative of the recipient
b. Urgent medical need, meaning no comparable human cell product was available and the recipient was likely to suffer death or serious morbidity without the human cell product, as attested by the Investigator
Aberrant CD45RA isoform expression by central laboratory testing
Women who were pregnant or breastfeeding

### Generation of Cell Components

Mobilization therapy included a 4-day regimen of granulocyte colony stimulating factor (G-CSF). For example, filgrastim subcutaneous injection, 10 g/kg of donor weight once daily for four days, starting from about 6 to 3 days prior to the recipient's conditioning regimen, and was determined on a case to case basis.

Large volume apheresis was performed. Two days of large volume apheresis may have been generally necessary to achieve sufficient HSPC for Recipient engraftment and/or sufficient Tmem for the assigned dose cohort. Per institutional standards, plerixafor (e.g., 0.24 mg/kg SC, once) was considered prior to the second apheresis if recommended by the Investigator to achieve the HSPC dose target.

TABLE 10 shows the Schedule of Activity of donors, including time points for screening, mobilization and apheresis.

TABLE 10
Schedule of Activity of Donors

| | Screenin | Mobilization | Apheresis | |
|---|---|---|---|---|
| Study Day (vs. recipient | −31 to −6 | −6 to −3 | −2 or −1 | −1 to 01 |
| conditioning) | | | | |
| Window² | | ±2 days | ±2 days | ±2 days |
| Informed consent | x⁸ | | | |
| Inclusion/Exclusion | X | | | |
| Criteria | | | | |
| Vital Signs⁴ | X | | X | X |
| CBC with differential⁴ | X | Daily x4 or | X | X |
| | | per | | |
| | | institutional | | |
| CD45 isoform testing per | X | | | |
| central | | | | |
| G-CSF mobilization | | Daily x4 | X | X |
| CD34 count (flow | | | X | |
| cytometry)⁷ | | | | |
| Apheresis | | | X | X |
| Blood for biomarker | X | | X | |
| analysis | | | | |
| Stool for biomarker | X | | | |
| analysis⁸ | | | | |

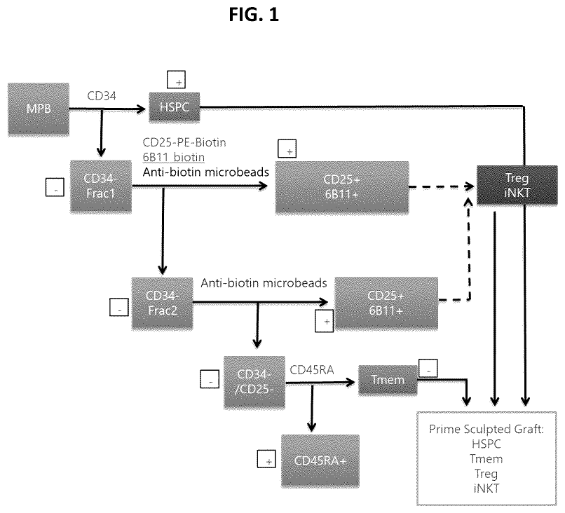

FIG. 1 illustrates a schematic of graft production and administration.

### Treatments

Provided in this section are examples of administration of a cellular therapy product of the present disclosure provided herein. In some embodiments, the composition of cells comprises one or more infusions. In some embodiments, the composition of cells comprises two infusions (e.g., primary product and product supplement). In some embodiments, the composition of cells was administered with concomitant therapy comprising myeloablative conditioning regiment.

### A. Administration of Cells

Dosing of the cellular therapy product of the present disclosure was based on a recipient's actual body weight rounded to the nearest tenth of a kilogram, assessed during screening. Dosing may have been adjusted to a dose based on adjusted ideal body weight (AIBW) if the patient's actual weight was greater than 120% of the ideal body weight (IBW). IBW was determined using the method of Devine (Devine 1974). AIBW (in kg) is equal to [(actual weight−IBW)×0.40]+IBW.

All recipients had appropriate long-term central venous access placed prior to initiation of the conditioning regimen. Premedication with acetaminophen and/or diphenhydramine was administered prior to administration of the cellular therapy product of the present disclosure, unless contraindicated, the cellular therapy product of the present disclosure was administered intravenously (IV) through a central venous catheter on study Day 0. If necessary to achieve target numbers of HSPC and/or Tmem, the product supplement was administered as soon as available and feasible after the cellular therapy product of the present disclosure on Day 0 or Day +1.

### B. Concomitant Therapy

All concomitant medications, blood products, procedures, radiotherapy and/or other treatments with therapeutic intent administered to recipients was recorded from the beginning of the conditioning regimen through the safety reporting period. Any concomitant therapy given for a study protocol-related adverse event was recorded from the time of informed consent. Any treatment for adverse events of special interest (AESI), disease recurrence/relapse, GVHD of any type, or PTLD was recorded at all times.

### Myeloablative Conditioning (MAC) Regimens

Prior to receiving the cellular therapy product of the present disclosure, subjects received one of the arm-specific myeloablative conditioning (MAC) regimens. Examples of MAC regiments for each arm are provided below in Table 11 and Table 12 Anti-Thymocyte Globulin (ATG) was allowed only as described herein. Doses and schedules varied slightly based on institutional practice.

TABLE 11
Arms A and C: Allowed Myeloablative Conditioning Regimens for
Recipients with 8/8 HLA-matched Related or Unrelated Donors

Busulfan/Fludarabine/Thiotepa (TBF)
Busulfan (9.6 mg/kg IV)
Fludarabine (150 mg/m²)
Thiotepa (10 mg/kg)
Total Body Irradiation/Fludarabine/Thiotepa (TBI/Flu/Thiotepa, TFT)
Fractionated TBI (1375 cGy with lung shielding; 600 cGy boost to
overlying ribs; male patients receive an additional 400 cGy testicular
boost in a single fraction)
Fludarabine (125 mg/m²)
Thiotepa (10 mg/kg)
Total Body Irradiation/Fludarabine (TBI/Flu)
Fractionated TBI (1350 cGy with lung shielding)
Fludarabine (160 mg/m2)

Patients with a Mismatched Unrelated Donor (7/8 HLA matched) or Haploidentical Related Donor (≥4/8 HLA matched) enrolled onto Arm A or Arm B received either one of the following regiments.
a. A modified Busulfan/Fludarabine/Thiotepa (BFT) regimen as described by Sanz et al. (Sanz 2010), with or without ultralow dose anti-thymoglobulin (ATG) modified from Djamali et al. (Djamali 2000) therapy described below. If BFT was used without ATG, the BFT regimen was accompanied by either
   timed-sequential dosing of busulfan (Popat 2018), or
   200-400 cGy Total Body Irradiation (TBI)
b. the TBI/Fludarabine/Thiotepa regimen described by Jakubowski et al. (Jakubowski 2007), or
c. a modified TBI/Fludarabine (TBI/Flu) described by Kanda et al. (Kanda, 2011), with or without ultralow dose anti-thymoglobulin (ATG) modified from Djamali et al. (Djamali 2000) therapy described below.

ATG: To reduce the incidence of graft failure when using the BFT or TBI/Flu regimens with alternative donors, ATG was administered. Specifically, Thymoglobulin® (Sanofi) was administered from 1.5 mg/kg to 7.5 mg/kg or dosed based on individualized PK-population methods based on the patient's weight and absolute lymphocyte count (Admiraal 2022). The ATG dose may have been divided over up to three days (Days −10 to −8) with no more than 2.5 mg/kg given per day. Premedication with corticosteroids, acetaminophen, and/or diphenhydramine prior to ATG administration was allowed per institutional practice.

Timed-sequential busulfan: This approach is an alternative to use of ultra-low dose ATG with BFT. As described by Popat et al (Popat 2018), on days −13 and −12, patients received 80 mg/m² busulfan intravenously (IV) daily in an outpatient clinic. Busulfan PK analysis was completed after the first dose on day −13, based on which all patients needed adjustments for day −6 and day −5 doses of busulfan. Target AUC 16000 μmolₘᵢₙ. Additional busulfan doses were administered during inpatient treatment from day −6 through day −3.

TABLE 12
Arm B: Allowed Myeloablative Conditioning Regimen

Busulfan/Fludarabine/Thiotepa (BFT + ATG)
Busulfan (9.6 mg/kg IV)
Fludarabine (150 mg/m²)
Thiotepa (10 mg/kg)
ATG administered D −8-D −10
Busulfan/Fludarabine/Thiotepa (BFT + 200-400 cGy TBI)
Busulfan (9.6 mg/kg IV)
Fludarabine (150 mg/m²)
Thiotepa (10 mg/kg)
200-400 cGy TBI
Note: ATG is not required with the BFT + TBI regimen
Total Body Irradiation/Fludarabine/Thiotepa (TBI/Flu/Thiotepa, TFT)
Fractionated TBI (1375 cGy with lung shielding; 600 cGy boost to
overlying ribs; male patients receive an additional 400 cGy testicular
boost in a single fraction)
Fludarabine (125 mg/m²)
Thiotepa (10 mg/kg)
Note: ATG is not required with the TFT regimen
Total Body Irradiation/Fludarabine (TBI/Flu + ATG)
Fractionated TBI (1350 cGy with lung shielding)
Fludarabine (160 mg/m²)
ATG administered D −8-D −10

### Single-Agent GVHD Prophylaxis

Recipients assigned to all three Arms received single-agent GVHD prophylaxis beginning on Day −1, consisting of tacrolimus. Post-transplant cyclophosphamide was not permitted.

Tacrolimus dose and tapering: For patients in Arms A and B, tacrolimus was initiated at 0.03 mg/kg/day IV, with a target trough blood level of 5-10 ng/mL. Per os (PO) administration was permissible when the patient was able to tolerate food. Recipients who demonstrated no evidence of Grade ≥II acute GVHD prior to Day +60 were able to taper the prophylaxis regimen at approximately 2000 of the dose per month. Initiating a GVHD prophylaxis regimen prior to Day +60 occurred in some cases. For recipients who developed Grade ≥II acute GVHD prior to Day +60, treatment of acute GVHD took precedence. For instance, completion of a corticosteroid taper was considered before tapering of tacrolimus.

### Antimicrobial Prophylaxis

All patients received prophylaxis against bacterial, fungal, and viral infections during the peri-transplant period according to institutional practices.

### Other Therapies

Additional therapies were allowed to be added to the treatment regimen for HCT-related symptom control or toxicity control, such as growth factors, intravenous immunoglobulin, blood product transfusions, analgesics, anti-emetics, electrolyte replacement and hydration. Other allowed therapy included vitamins, nutritional supplements, drugs to prevent cancer relapse (e.g., FLT3 or other tyrosine kinase inhibitors), corticosteroids, and anti-infective agents.

### Outcome Assessments

Subjects enrolled in the study were clinically assessed prior to, during, and after the administration of the cell components provided herein. Recipient assessment were done following TABLE 9 (A-C) Schedule of Activity. The following paragraphs provide data and explanations related to clinical outcomes from the clinical studies described herein. As an introductory matter in the explanation of the data, it is to be recognized that graft failure can be a complication of HCT, and can be associated with significant mortality.

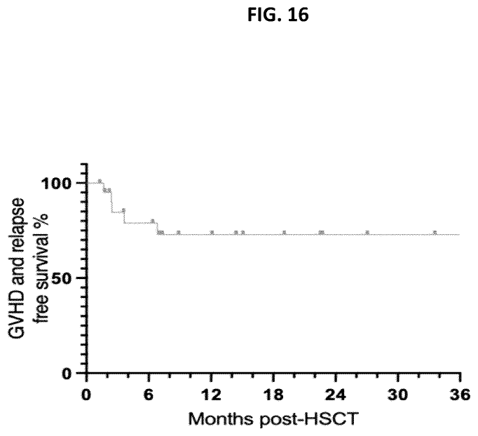

The drug product that comprises the cell populations comprising CD34+ HSPC enriched with Treg and depleted of naïve conventional αβ-T cells was successfully manufactured and delivered to all subjects with a vein-to-vein time of less than 72 hours. Data from all arms are explained in detail below. For example, in Arm B, a total of 21 patients (11 AMIL, 8 ALL, 2 CML in blast crisis) with >30 days of follow up were included in this analysis. All were 4/8 HLA match at HLA-A, -B, C, and DRB1. Median age of subjects was 44 years (range 21-63) and median follow-up was 333 days (range 39-1020). Disease risk index (DRI) score was available in 20 showing 2 very high, 3 high, 13 intermediate, and 2 low risk categories. The M:F ratio was 12:9. Patients received either TBI (n=13) or non-TBI (n=8) based MAC regimens. 2 patients had Gr 1 CRS (CTCAE v5). Median neutrophil and platelet engraftment times were 13 days (range 8-25) and 16 days (range 12-25), respectively. 1 secondary engraftment failure was observed. The estimated incidence of BMT CTN MOP3 infections was 27% at 1 year. The estimated incidence of gr 2-4 aGVHD at 6 months was 14% which compares favorably with literature reports of 21% to 63% for gr 2-4 aGVHD in patients undergoing haplo transplant with PTCy-based prophylaxis.[1-5] Likewise, gr 3-4 aGVHD was rare with the cellular therapy product of the present disclosure with only 1 case of gr 3 aGVHD and no gr 4 aGVHD. Of the 16 patients who had at least 3 months follow up, only 1 developed mild cGVHD. No patients had moderate-to-severe cGVHD, a notable improvement compared to historic rates of 24% to 31% for moderate-to-severe cGVHD with PTCy, despite the fact that patients receiving the cellular therapy product of the present disclosure receive only single agent GVHD prophylaxis.[1-4]2 patients experienced disease relapse and there was a total of 5 deaths (2 due to relapse, 1 Aspergillus pulmonary infection, 1 respiratory failure associated with renal failure, and 1 COVID infection). GVHD-and-relapse free survival (GRFS) was 71% at 1 year with the cellular therapy product of the present disclosure (FIG. 16) which compares favorably to a GRFS of 46% reported by Sanz in the context of MAC haplo alloHCT with PTCy.

Initial data demonstrates good clinical outcomes with single agent tacrolimus and no PTCy or MMF. Patients treated with haplo alloHCT and the cellular therapy product of the present disclosure experienced a low adverse event profile, low incidence and severity of both aGVHD and cGVHD, and improved GRFS rates, offering a potential new treatment option for patients undergoing haplo alloHCT.

### Engraftment

Neutrophil engraftment through Day +28: Neutrophil engraftment was defined as achieving an absolute neutrophil count (ANC)≥500/mm3 for 3 consecutive days, by Day +28. The first of the three days was designated the day of engraftment. If ANC never dropped below 500/mm3, Day +1 was assigned as the day of engraftment.

Platelet engraftment through Day +50: Platelet engraftment was defined as achieving a platelet count >20,000/mm3 for 3 consecutive days without platelet transfusion in the preceding 7 days, by Day +50. The first of the three days was designated the day of engraftment. If platelet count never dropped below 20,000/mm3, Day +1 was assigned as the day of engraftment.

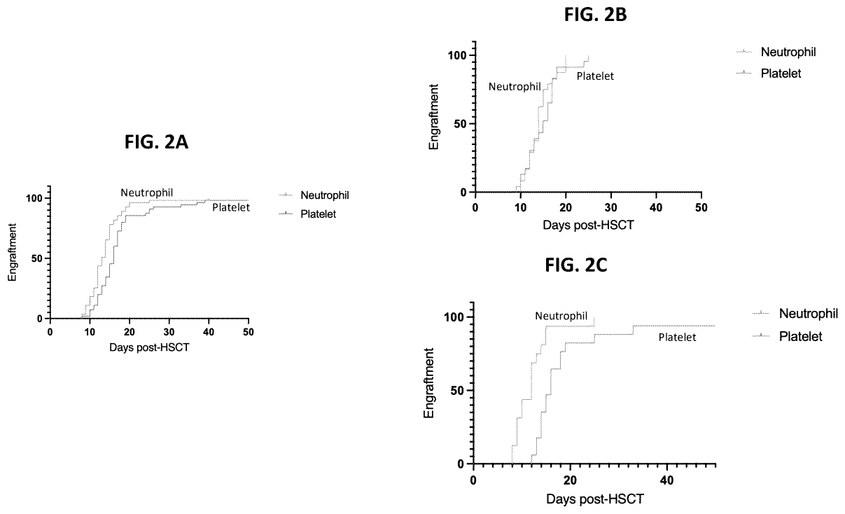

FIG. 2A illustrates neutrophil and platelet engraftment in all cohorts of the study. Median days of neutrophil engraftment in this cohort is 13 days (4 to 40 day range). Median days of platelet engraftment in this cohort is 16 days (11 to 79 day range). FIG. 2B illustrates neutrophil and platelet engraftment in the MRD/MUD cohort of subjects in Arm A of the study. Median days of neutrophil engraftment in this cohort is 14 days (9 to 20 day range). Median days of platelet engraftment in this cohort is 15 days (10 to 25 day range). FIG. 2C illustrates neutrophil and platelet engraftment in Arm B of the study. Median days of neutrophil engraftment in this cohort is 13 days (8 to 25 day range). Median days of platelet engraftment in this cohort is 16 days (12 to 25 day range). As illustrated in FIGS. 2A-2C, the pharmaceutical treatment used in this study achieved faster neutrophil and platelet engraftment in Arm B (e.g., haploidentical related donor group).

### GVHD Evaluation, Cancer Relapse, and Survival

Acute GVHD (aGVHD) is a significant driver of morbidity and mortality associated with alloHCT, reducing the severity and incidence of aGVHD has the potential to greatly benefit graft recipients. Acute GVHD was staged and graded per MAGIC Standardization criteria. Chronic GVHD was diagnosed, staged and graded per the International NIH Chronic GVHD Diagnosis and Staging Consensus Working Group criteria. Clinically significant manifestations of both acute and chronic GVHD were treated first by local, topical, and/or systemic corticosteroids (e.g., prednisone). Any patients who were refractory or resistant to, dependent upon, or intolerant of corticosteroids, per BMT-NIH-CIBMTR Task Force definition, were to be considered for second line therapy. Treatment-emergent adverse events (TEAEs): TEAEs were categorized by System Organ Class and preferred term using MedDRA version 21.0 and were graded according to the CTCAE version 5.0.

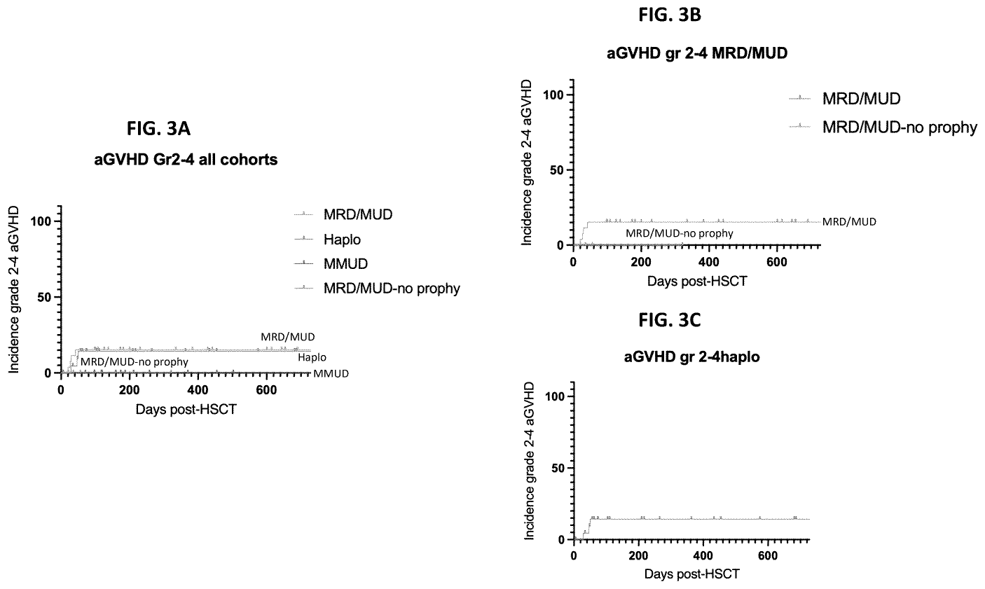

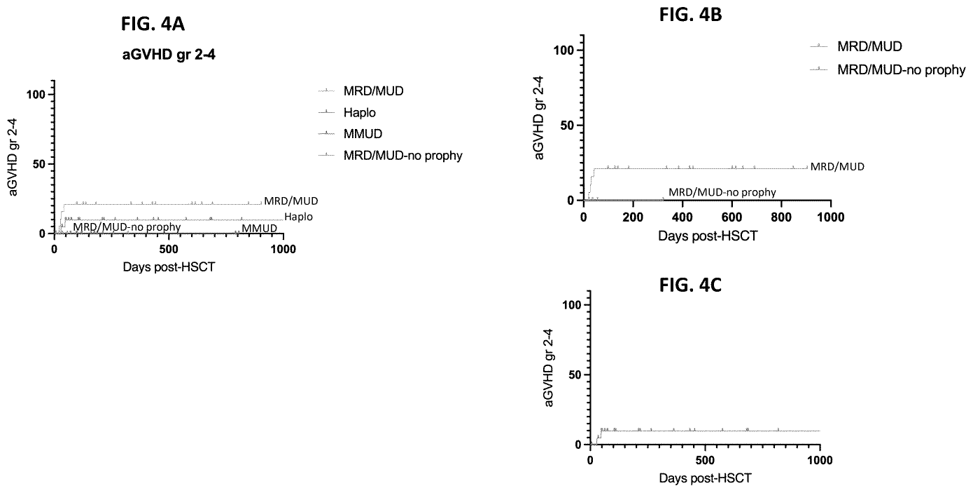

Subjects were considered evaluable for aGVHD if they developed aGVHD symptoms before day +100 (100 days post-transplant), or were beyond day +100 without exhibiting aGVHD symptoms. FIGS. 3A-3C depict the incidence of grade 2-4 aGVHD in different cohorts of the study. FIG. 3A depicts the incidence of grade 2-4 aGVHD in all cohorts (e.g., MRD/MUD, and MMUD in ArmA; Haplo in ArmB; and no-GVHD prophylaxis in ArmC), and FIG. 3B and FIG. 3C illustrate the said incidence in all recipients in ArmA/C and ArmB respectively. Recipients that received no-GVHD prophylaxis showed the lowest incidence of grade 2-4 aGVHD. FIGS. 4A-4C depict the incidence of grade 2-4 aGVHD in recipients received over 1×10⁶ Tmem per kg of recipient subject's actual body weight or (adjusted) ideal body weight in different cohorts of the study. FIG. 4A depicts said incidence in recipients that received over 1×10⁶ Tmem per kg of ABW or (adjusted) IBW in all cohorts, and FIG. 4B and FIG. 4C depict the said incidence in said recipients in Arm A/C and Arm B respectively. Recipients that received no-GVHD prophylaxis showed the lowest incidence of grade 2-4 aGVHD.

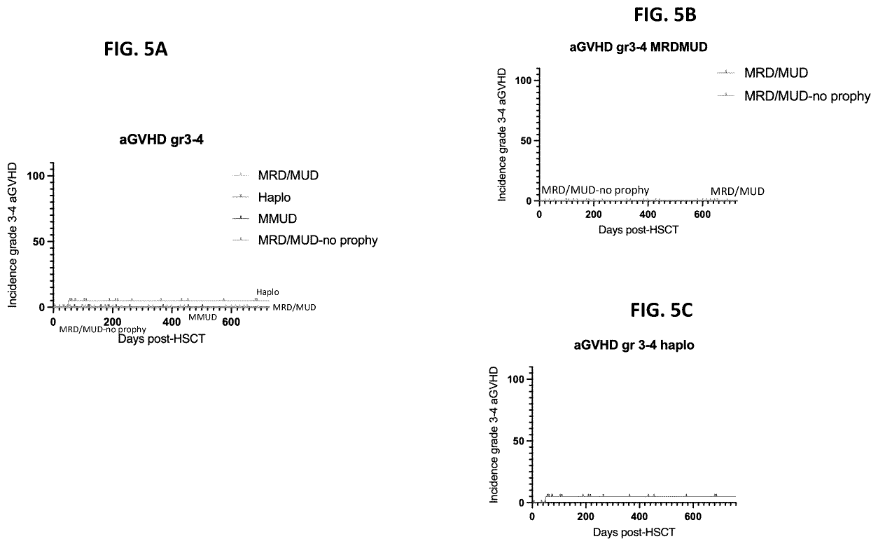

Severe (Grade 3-4) aGVHD is a major contributor to non-relapse mortality post-alloHCT and can be observed in 10-20% of patients following an HLA-matched, related donor transplants. FIGS. 5A-5C depict the incidence of grade 3-4 aGVHD in different cohorts of the study. FIG. 5A depicts the incidence of grade 3-4 aGVHD in all cohorts, and FIG. 5B and FIG. 5C illustrate the said incidence in all recipients in ArmA/C and ArmB respectively. These results suggest a composition of the disclosure can achieve low incidence and severity of aGVHD in recipients, even in the absence of GVHD prophylactics or with reduced GVHD prophylaxis.

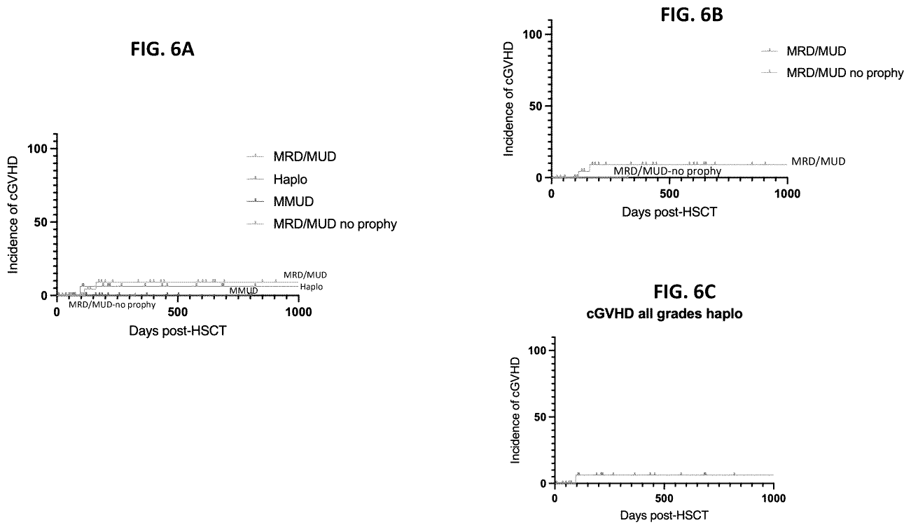

As chronic GVHD (cGVHD) is associated with significant morbidity and with decreased survival, reducing the severity or incidence of cGVHD has the potential to greatly benefit graft recipients. FIGS. 6A-6C depict the incidence of chronic GVHD of all grades in different cohorts of the study. FIG. 6A depicts the incidence of chronic GVHD of all grades in all cohorts, and FIG. 6B and FIG. 6C illustrate the said incidence in all recipients in ArmA/C and ArmB respectively. All cGVHD observed were mild cases. As cGVHD is associated with decreased overall survival and significant long-term morbidity, these results suggest that a composition of the disclosure can improve long-term survival and quality of life in recipient subjects.

Subjects were monitored for survival and relapse.

Incidence of disease relapse: For acute leukemias, relapse was defined as any of the following (MRD+alone was insufficient): (i) ≥5% blasts in the bone marrow or peripheral blood; or (ii) Reappearance of pre-transplant cytogenetic abnormality; or (iii) new evidence or redevelopment of extramedullary disease. For MDS, relapse was defined as any of the following: (i) satisfying criteria for evolution into acute leukemia; (ii) reappearance of pre-transplant morphologic abnormalities, detected in bone marrow specimens; or, (iii) reappearance of pre-transplant cytogenetic abnormality in at least one metaphase on each of two separate consecutive examinations at least one month apart, regardless of the number of metaphases analyzed.

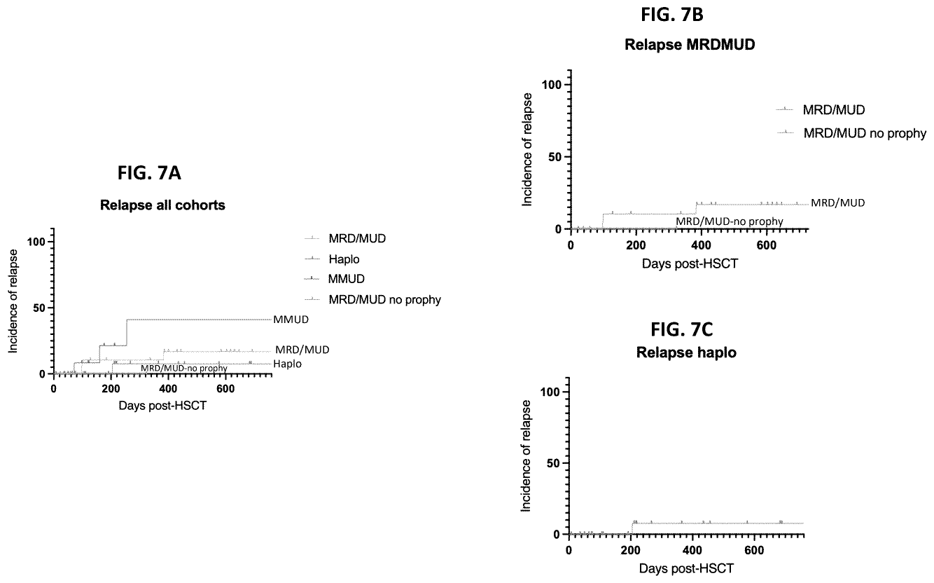

FIGS. 7A-7C depict the incidence of disease relapse in different cohorts of the study. FIG. 7A depicts the incidence of disease relapse in all cohorts, and FIG. 7B and FIG. 7C illustrate the said incidence in all recipients in ArmA/C and ArmB respectively.

Incidence of non-relapse mortality (NRM). NRM was defined as death without evidence of disease recurrence. Disease relapse/progression was considered a competing event.

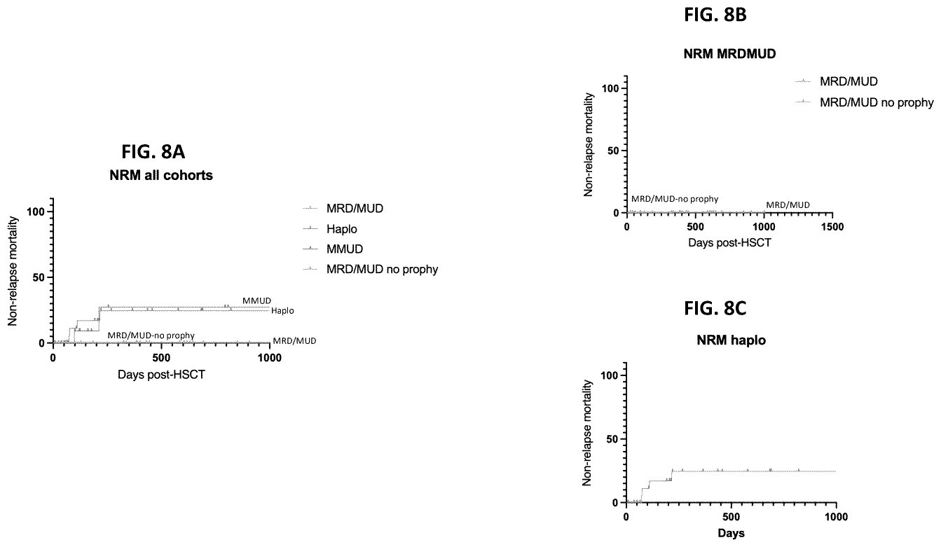

FIGS. 8A-8C depict the incidence of NRM in different cohorts of the study. FIG. 8A depicts the incidence of NRM in all cohorts, and FIG. 8B and FIG. 8C illustrate the incidence of NRM in all recipients in ArmA/C and ArmB respectively.

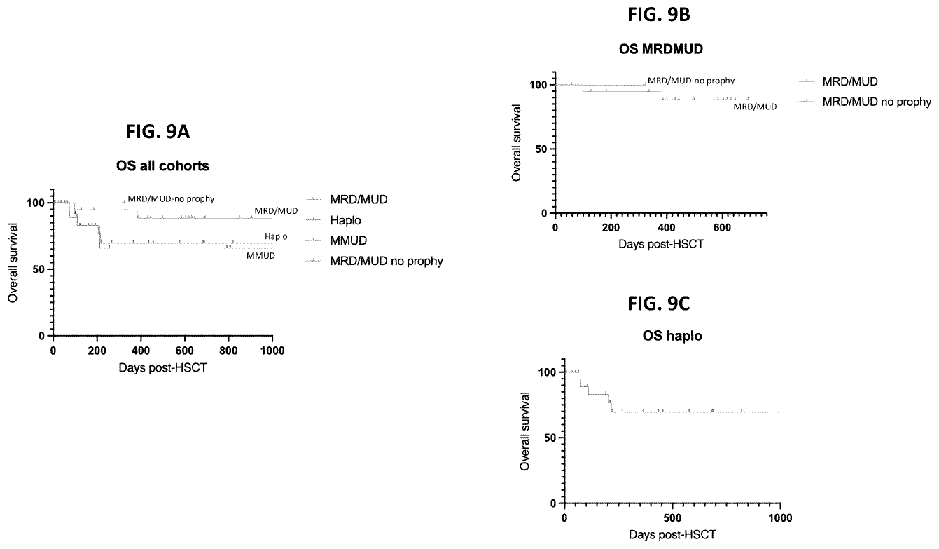

Overall survival (OS) was defined as the time from the date of transplant to the date of death from any cause or, for surviving patients, to the date of last follow-up. FIGS. 9A-9C depict the OS in different cohorts of the study. FIG. 9A depicts the OS in all cohorts, and FIG. 9B and FIG. 9C illustrate the OS in all recipients in ArmA/C and ArmB respectively.

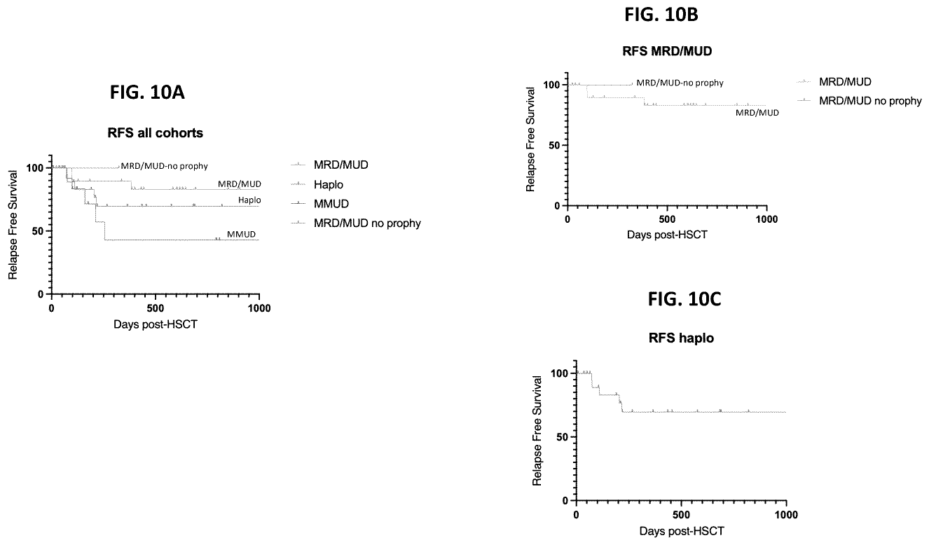

Relapse-free survival (RFS) is a readout that can refer to survival rate without disease relapse. FIGS. 10A-10C depict the RFS in different cohorts of the study. FIG. 10A depicts the RFS in all cohorts, and FIG. 10B and FIG. 10C illustrate the RFS in all recipients in ArmA/C and ArmB respectively.

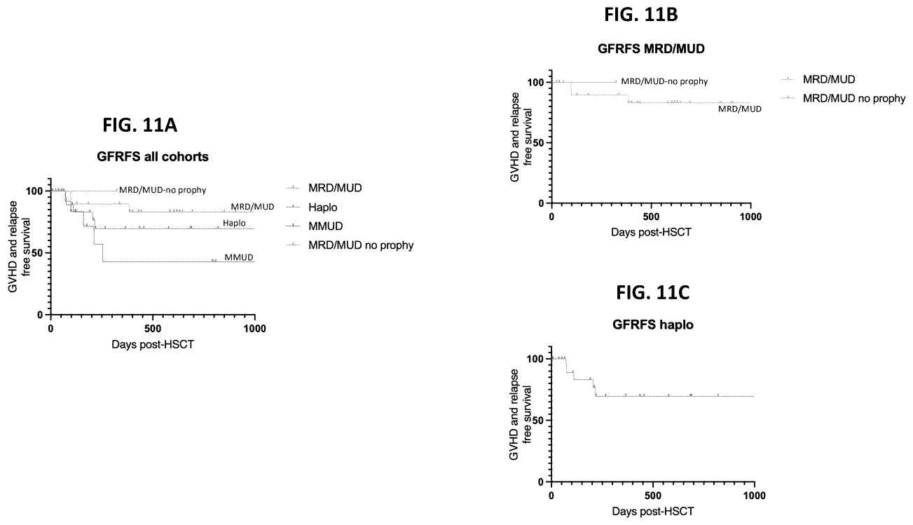

GVHD and relapse-free survival (GRFS) is a composite readout that can refer to survival without relapse or Grade ≥3 acute or extensive chronic GVHD. FIGS. 11A-11C depict the GRFS in different cohorts of the study. FIG. 11A depicts the GRFS in all cohorts, and FIG. 11B and FIG. 11C illustrate the GRFS in all recipients in ArmA/C and ArmB respectively.

In summary, recipient subjects were evaluated and clinical outcomes comprising graft failure, aGVHD, cGVHD, cancer relapse, and survival were analyzed at the time of reporting. TABLE. 13 is a summary of the data collected from subjects that received over 1×10⁶ Tmem per kg of recipient subject's actual body weight or (adjusted) ideal body weight.

TABLE 13

| | | Median followup | | | | | |
|---|---|---|---|---|---|---|---|
| | N | days | GF | aGVHD | cGVHD | Relapse | Death |
| ARM A: | 19 | 379 (98-1011) | 1 | 5 (2 gr 1, | 2 (2 mild) | 3 | 2 (1 disease prog, 1 e. coli |
| MRD/MUD | | | | 3 gr 2) | | | sepsis with relapse) |
| ARM A: | 12 | 332 (71-1069) | 0 | 3 (2 gr 1, | 0 | 4 | 3 (1 relapse, 1 MOF, |
| MMUD | | | | 1 gr 2) | | | 1 COVID) |
| ARM B: | 22 | 297 (10-1020) | 1 | 4 (1 gr 1, | 1 (1 mild) | 1 | 5 (1 relapse, 1 shock, 1 |
| Haplo | | | | 2 gr 2, | | | Aspergillus, 1 resp failure, |
| | | | | 1 gr 3) | | | 1 COIVD) |
| ARM C; | 4 | 126 (30-325) | 0 | 0 | 0 | 0 | 0 |
| MRD/MUD no | | | | | | | |
| GVHD prophy | | | | | | | |

### Infection

Serious infections can occur at alloHCT settings partially due to procedures such as conditioning and administering GVHD prophylactic agents. Opportunistic infections can be severe and lethal. All recipient subjects received prophylaxis against bacterial, fungal, and viral infections during the peri-transplant period according to institutional practices. Infections can be graded based on the Technical Manual of Procedures by the Blood and Marrow Transplant Clinical Trials Network (BMT CTN MOP).

The incidence of infection was monitored and infections were treated based on clinical guidelines. Tmem have been implicated in the promotion of graft-versus-infection and graft-versus-leukemia effects, and the cell populations comprising Tmem administered to the recipient subjects in the study can lower the rate of infection.

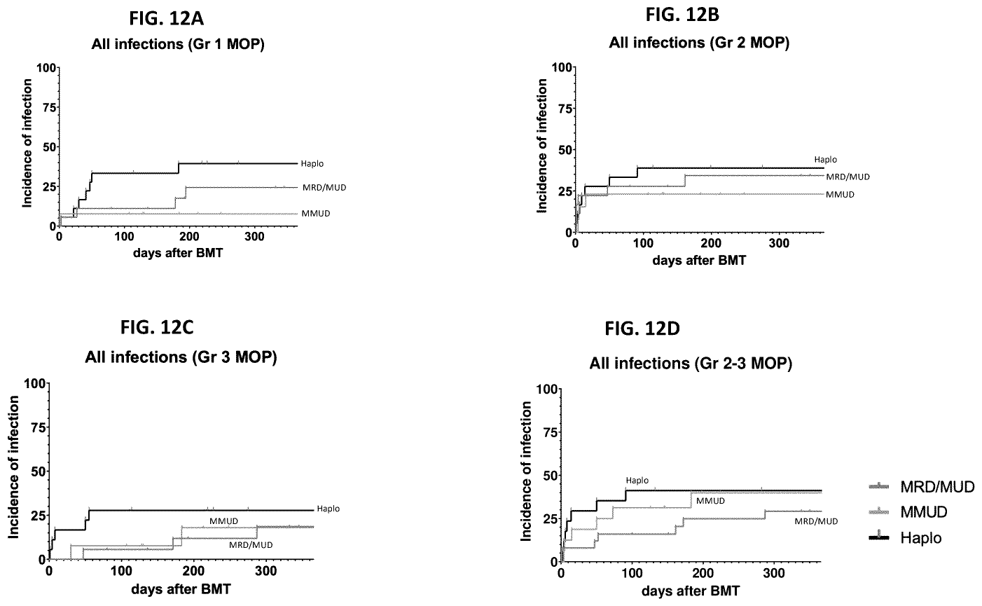

FIGS. 12A-12D depict the incidence of all infections in various arms of the study at about a year after the transplant. FIG. 12A, FIG. 12B, FIG. 12C, and FIG. 12D depict the incidence of BMT CTN MOP Grade 1, Grade 2, Grade 3, and Grade 2-3 infections respectively.

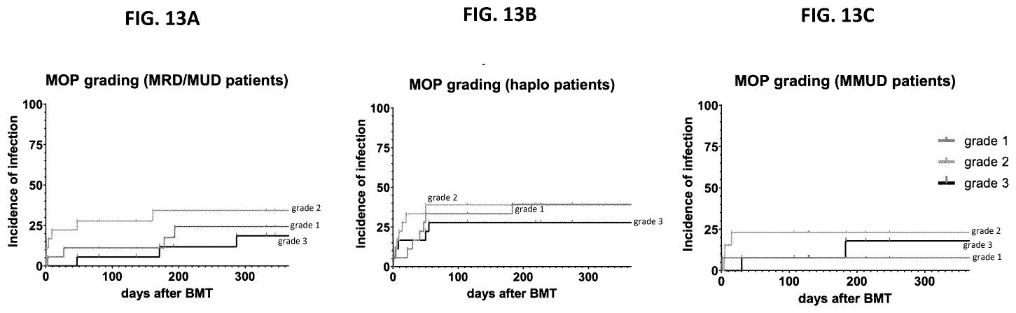

FIGS. 13A-13C depict the incidence of all infections in various cohorts of the study at about a year after the transplant. FIG. 13A, FIG. 13B, and FIG. 13C depict the incidence of all grades of infections in the MRD/MUD cohort, the haploidentical cohort (ArmB), and the MMUD cohort respectively.

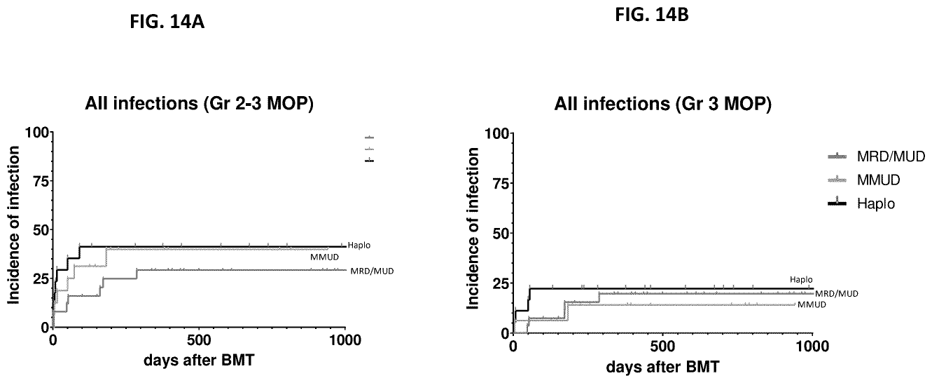

FIGS. 14A-14B depict the incidence of infections in various arms of the study at about 1000 days after the transplant. FIG. 14A and FIG. 14B depict the incidence of BMT CTN MOP Grade 3 and Grade 2-3 infections respectively.

TABLE 14 summarizes the rate of viral, bacterial and fungal infections in the recipient subjects.

TABLE 14

| | | | MRD/ | | |
|---|---|---|---|---|---|
| | | | MUD | MMUD | Haplo |
| Viral | Herpesviruses | VZV | 4% | | |
| | | EBV | 4% | | 5% |
| | | CMV | 7% | 6% | 20% |
| | | HHV6 | | | 5% |
| | Coronavirus | COVID-19 | | 19% | 10% |
| | Rhinovirus | Rhinovirus | 7% | | |
| | Polyomavirus | JC/BK | 4% | | 5% |
| Bacterial | Confirmed | C. diff | 7% | | 20% |
| | etiology | Staph/Strep | 7% | 6% | 5% |
| | | GNR | 11% | 12% | 15% |
| | Unconfirmed | Pneumonia | | 6% | 10% |
| | etiology | Sepsis | 15% | 6% | 5% |
| | | Skin | 11% | 6% | 5% |
| | | infection | | | |
| Fungal | Superficial | Candidiasis | 7% | 12% | 15% |
| | mycosis | Tinea cruris | | | 5% |
| | Systemic | Aspergillus | | | 5% |
| | mycosis | | | | |

### Example 2: Comparison with Results in the Literature

Clinical assessment outcomes of this study were compared with outcomes from previous studies in the field, and the pharmaceutical treatment, method, and regiment described herein achieved superior results.

Compared to the study published in Slade, M., et al., (Epidemiology of infections following haploidentical peripheral blood hematopoietic cell transplantation. Transpl Infect Dis, 2017. 19(1), which is incorporated herein by reference in its entirety), which analyzed haploidentical recipients who received a different cell population than that described herein and treated with the single GVHD prophylactic agent PTCy, the present study achieved superior clinical outcomes in a similar subject population, such as a lower CMV infection rate. TABLE 15 illustrates the rate of infection in ArmB of the present study and in Slade et al 2017. The CMV infection rate (20%) in Arm B of the present study is significantly lower than that of other studies related to HSPC transplant.

TABLE 15

| | | | Cellular | |
|---|---|---|---|---|
| | | | Therapy | Slade et al |
| | | | Product -Haplo | 2017[1] |
| Viral | Herpesviruses | VZV | 0 | 1% |
| | | EBV | 5% | 0 |
| | | CMV | 20% | 54.8% |
| | | HHV6 | 5% | 0 |
| | | HSV1/2 | 0 | 6.7% |
| | Coronavirus | COVID-19 | 10% | 0 |
| | Respiratory | Influenza, | 0 | 31.7% |
| | viruses | RSV, | | |
| | | Parainfluenza | | |
| | Polyomavirus | JC/BK | 5% | 19.2% |
| Bacterial | Confirmed | C. diff | 20% | 12.5% |
| | etiology | Staph/Strep | 5% | 24% |
| | | GNR | 15% | 28.8% |
| | Unconfirmed | Pneumonia | 10% | N/A |
| | etiology | Sepsis | 5% | N/A |
| | | Skin infection | 5% | N/A |
| Fungal | Superficial | Candidiasis | 15% | 1.9% |
| | mycosis | Tinea cruris | 5% | N/A |
| | Systemic | Aspergillus | 5% | 1.9% |
| | mycosis | | | |

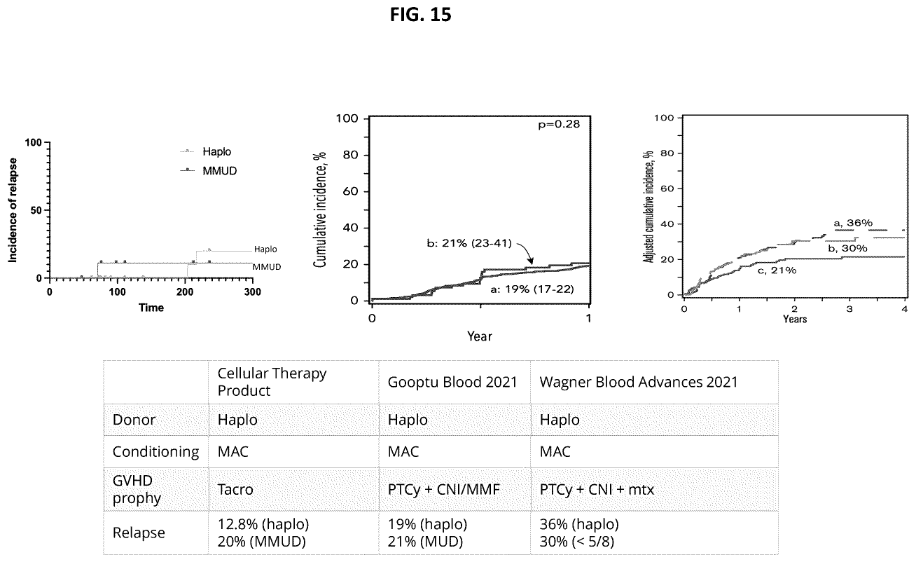

Incidence of relapse in some cohorts of the present study were comparable or lower than published literature. FIG. 15 compares the incidence of relapse and study design of the present study to that of Gooptu (Blood 2021) and Wagner (Blood Advances 2021). As depicted in FIG. 15, Arm B (haplo) of the present study achieved a relapse rate of 12.8%, lower than that of Gooptu (18%) and that of Wagner (36%). As depicted in FIG. 15, the MMUD cohort of the present study achieved a relapse rate of 20%, lower than that of Gooptu (21%) and that of Wagner (30%). TABLE 16 compares clinical outcomes from the present study with that of other studies, such as Gooptu (Blood 2021).

TABLE 16

| | | Grade | Grade | | | | | | |
|---|---|---|---|---|---|---|---|---|---|
| | | 2-4 | 3-4 | | | | | | |
| | GF | aGVHD | aGVHD | cGVHD | NRM | Relapse | RFS | GRFS | OS |
| Orca-Q (present | 4.5% | 14% | 4.5% | 4.5% | 14% | 9% | 68% | 68% | 68% |
| study) | | | | | | | | | |
| Bashey 2016 n = 116 | 2.6% | 41% at | 17% at | 31% mod-sev | 17% 2 yr | 29% 2 yr | 54% 2 | | 57% 2 |
| single center data | | 6 mos | 6 mos | 2 yr 25% in | | | yr | | yrs |
| evaluation | | | | PB mod-sev | | | | | |
| Gooptu, 2021 | 4% | 33% | 10% | 33% 1 yr | 15% 1 yr | 19% 1 yr | 66% 1 | | 75% 1 |
| PB + BM (n = 825) | | | | | | | yr | | yr |
| CIBMTR data- | | | | | | | | | |
| observational | | | | | | | | | |
| Wagner 2021 PB + | | 21-30% | 6% | 22-48% 3 yr | 6-14% 3 yr | 35% 3 yr | 59-51% | | 71-60% |
| BM n = 375 CIBMTR | | | | | | | 3 yr | | 3yr |
| study | | | | | | | | | |
| Baker 2016 n = 54 | 7.4% | 63% 2 yr | 13% | 32% 2 yr | TRM-24% 1 yr | | | | 45% 2 |
| single center | | | 2 yr | 24% mod-sev | | | | | yr |
| | | | | 2 yrs | | | | | |
| Sanz 2020, n = 789 | 6% | 26% | 9% | 30% 2 yr | | 23% | 54% | 46% | 61% |
| EBMT database | | | | | | | | | |

### Example 3: Additional Clinical Results

The protocol, materials, and methods described in Example 1 were used to obtain the results described herein in Example 3.

As of 24 Mar. 2023, 81 participants have been treated with a cellular therapy product of the present disclosure in a single phase 1 study. The study is a multicenter open-label, phase 1 dose escalation and expansion study to assess the safety and tolerability of the cellular therapy product in adults undergoing myeloablative conditioning-allogeneic hematopoietic cell transplantation (MAC-alloHCT), with or without GVHD prophylaxis. Participants undergoing MAC-alloHCT for the treatment of acute myeloid, lymphoid, or undifferentiated leukemia; chronic myeloid leukemia, high- or very high-risk MDS; or myelofibrosis were enrolled into 1 of the 3 study arms as described in Example 1.

Upon enrollment of both an appropriate donor and recipient, the cellular therapy product of the present disclosure was engineered from the donor's mobilized peripheral blood. The recipient underwent a myeloablative conditioning (MAC) regimen followed by intravenous administration of the cellular therapy products, which included up to 2 product infusions (a primary product and a supplement product, the latter as needed to provide sufficient HPSCs and/or Tₘₑₘ), accompanied by either single-agent GVHD prophylaxis with tacrolimus (arms A and B) or no GVHD prophylaxis (arm C). Safety and efficacy outcomes were assessed for 1 year following administration of the cellular therapy product.

A total of 84 participants were enrolled in the study as of the data cutoff date of 24 Mar. 2023. A cellular therapy product of the present disclosure could not be produced for 3 participants due to poor donor mobilization. In 2 cases, poor donor mobilization was noted early in the process, and the apheresis product was not shipped for manufacturing. In 1 other case (a participant in arm B), the apheresis product was shipped for manufacturing prior to the recognition of poor donor mobilization. After limited processing, the cellular material derived from the apheresis product was returned to the participant's transplant center for infusion. This participant did not receive the cellular therapy product but was included in the safety analysis set for all data described herein.

Of the 82 participants in the safety analysis set, 19 participants (23%) completed the study, 22 participants (27%) withdrew from the study, and 41 participants (50%) were ongoing as of the data cutoff date. Five participants (6%) died before completing the study, 5 participants (6%) withdrew due to adverse events (AEs), and 2 participants (2%) withdrew consent. Ten participants (12%) withdrew for other reasons: 6 participants due to disease relapse followed by initiation of salvage therapy for relapse, 3 participants to start other anticancer therapy, and 1 participant did not have product manufactured due to poor donor mobilization.

The median age of participants was 43 years (range: 21 to 65 years), 61% of participants were male, and 57% of participants were white. Seventeen percent of participants reported their race as “other,” 13% of participants were black or African American, and 12% of participants were Asian.

The primary disease was AML for 50 participants (61%), ALL for 23 participants (28%), and MDS for 9 participants (11%). Of the 49 participants with AML, 37 (76%) were in their first complete remission (CR), and 9 (18%) were in their second CR at the time of transplantation. Of the 23 participants with ALL, 17 (74%) were in their first CR and 6 (26%) were in their second CR at the time of transplantation.

### Overall Survival

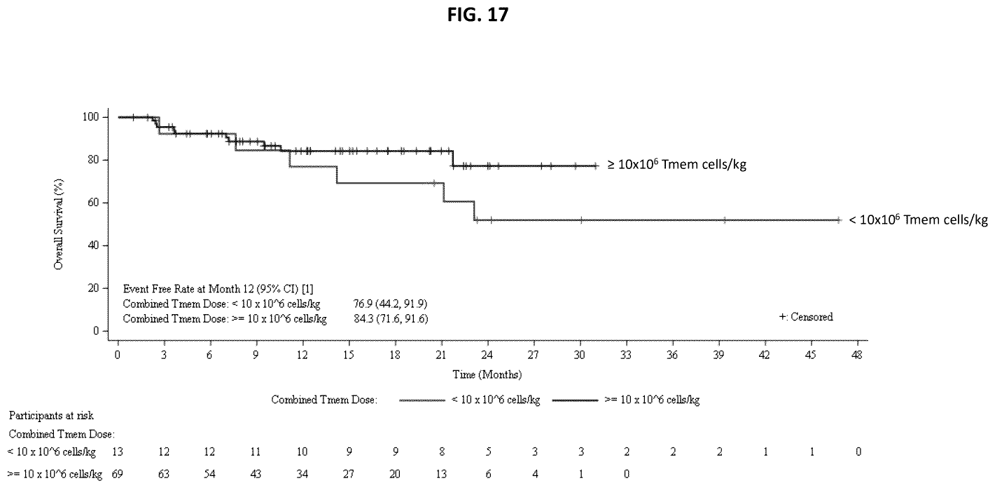

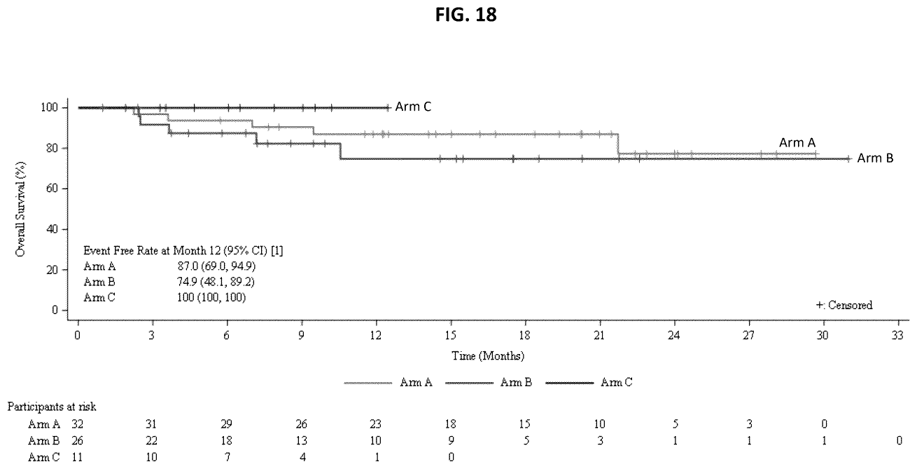

FIG. 17 shows Kaplan-Meier (KM) plots of overall survival (OS) by combined Tₘₑₘ dose (<10×10⁶ cells/kg versus 10×10⁶ cells/kg) and FIG. 18 shows KM plants for each study arm for participants who received a combined Tₘₑₘ dose of 10×10⁶ cells/kg. In the figures, “CI” refers to confidence interval, “KM” refers to Kaplan-Meier, “OS” refers to overall survival, and “Tmem” refers to memory T cells. OS is depicted as the time from first study treatment to death due to any cause. Participants alive or lost to follow-up were censored at the last date known to be alive or last follow-up date, whichever was later. Point estimates at specific months were based on the KM method. The 95% confidence intervals (95% Cis) for the event-free probability at specific months were computed using Greenwood's formula.

As shown in FIG. 17, the KM estimate for OS at 12 months was 77% (95% confidence interval [CI]: 44%, 92%) for participants who received a combined Tmem dose of <10×10⁶ cells/kg (n=13) and 84% (95% CI: 72%, 92%) for participants who received a combined Tₘₑₘ dose of 10×10⁶ cells/kg (n=69).

Among the 69 participants who received a combined Tmem dose of 10×10⁶ cells/kg, the KM estimate for OS at 12 months was 87% (95% CI: 69%, 95%) for arm A, 75% (95% CI: 48%, 89%) for arm B, and 100% (95% CI: 100%, 100%) for arm C (see FIG. 18). The KM estimate of median OS in months was not reached for all 3 arms.

### GVHD-Free and Relapse-Free Survival (GRFS)

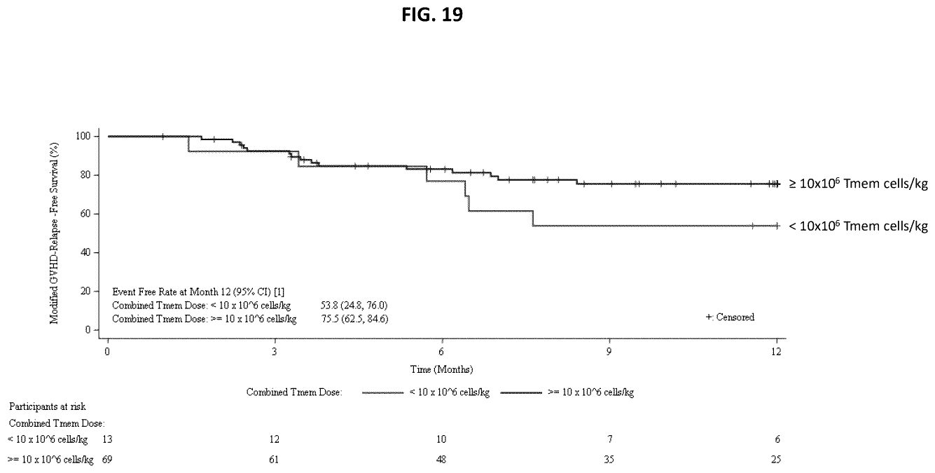

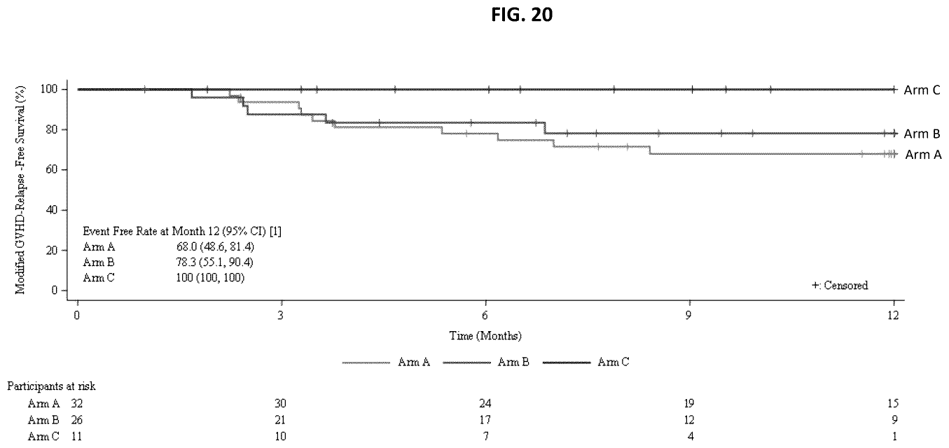

FIG. 19 shows KM plots of GVHD-free and relapse-free survival (GRFS) by combined Tₘₑₘ dose (<10×10⁶ cells/kg versus 10×10⁶ cells/kg) and FIG. 20 shows KM plots of GRFS by study arm for participants who received a combined Tₘₑₘ dose of 10×10⁶ cells/kg. In the figures, “aGVHD” refers to acute GVHD, “cGVHD” refers to chronic GVHD, “CI” refers to confidence interval, “GRFS” refers to GVHD-free and relapse free survival, “GVHD” refers to graft-versus-host disease, “KM” refers to Kaplan-Meier, and “Tmem” refers to memory T cells. Modified GRFS is depicted as the time from the date of transplant to death, grade 3 or 4 aGVHD/moderate or severe cGVHD, or relapse, whichever comes first. Participants who were alive and free from disease relapse or grade 3 or 4 aGVHD/moderate or severe cGVHD were censored at the date last follow-up date or last disease assessment, whichever was later. If the event or censoring date was after the end of follow-up, GRFS was censored at the end of follow-up. Point estimates at specific months were based on the KM method. The 95% CIs for the event-free probability at specific months were computed using Greenwood's formula.

As shown in FIG. 19, the KM estimate for GRFS at 12 months was 54% (95% CI: 25%, 76%) for participants who received a combined Tₘₑₘ dose of <10×10⁶ cells/kg (n=13) and 76% (95% CI: 63%, 85%) for participants who received a combined Tₘₑₘ dose of 10×10⁶ cells/kg (n=69).

Among the 69 participants who received a combined Tₘₑₘ dose of 10×10⁶ cells/kg, the KM estimate for GRFS at 12 months was 68% (95% CI: 49%, 81%) for arm A, 78% (95% CI: 55%, 90%) for arm B, and 100% (95% CI: 100%, 100%) for arm C (see FIG. 20). The KM estimate of median GRFS was not reached for all 3 arms.

### Relapse-Free Survival (RFS)

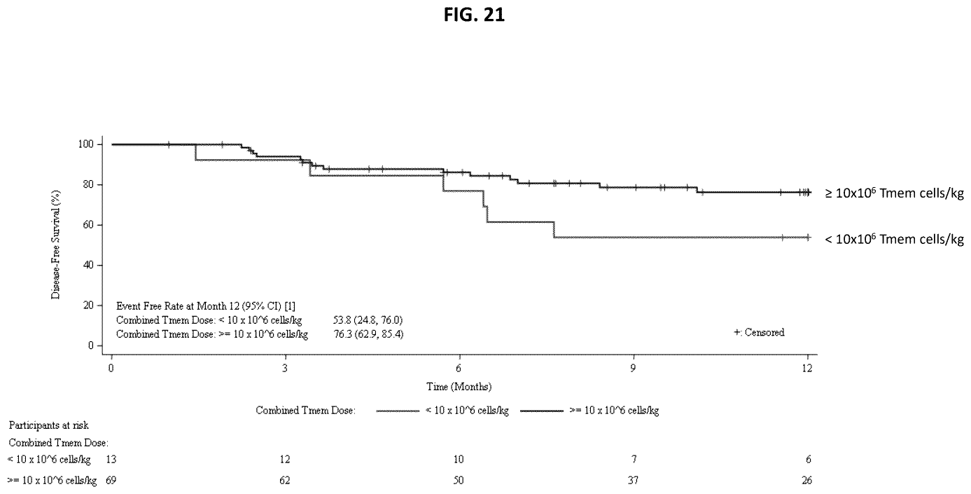

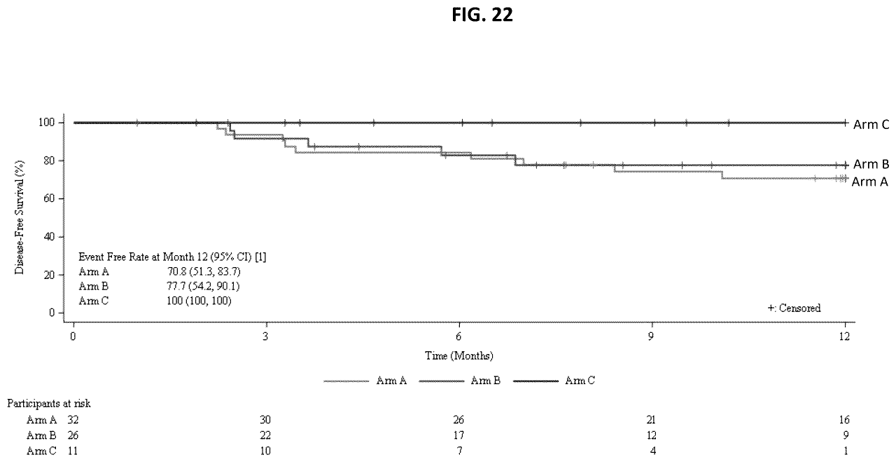

FIG. 21 shows KM plots of relapse-free survival (RFS) by combined Tₘₑₘ dose (<10×10⁶ cells/kg versus 10×10⁶ cells/kg) and FIG. 22 shows KM plots for RFS by study arm for participants who received a combined Tₘₑₘ dose of 10×10⁶ cells/kg. In the figures, “CI” refers to confidence interval, “RFS” refers to disease-free survival, “KM” refers to Kaplan-Meier, and “Tₘₑₘ” refers to memory T cells. DFS is depicted as the time from the date of transplant to death or relapse, whichever comes first. Participants who were alive and free from disease relapse were censored at their last follow-up visit or last disease assessment, whichever was later. If the event or censoring date was after the end of follow-up, DFS was censored at the end of follow-up. Point estimates at specific months were based on the KM method. The 95% CIs for the event-free probability at specific months were computed using Greenwood's formula.

As shown in FIG. 21, the KM estimate for RFS at 12 months was 54% (95% CI: 25%, 76%) for participants who received a combined Tₘₑₘ dose of <10×10⁶ cells/kg (n=13) and 76% (95% CI: 63%, 85%) for participants who received a combined Tₘₑₘ dose of 10×10⁶ cells/kg (n=69).

Among the 69 participants who received a combined Tₘₑₘ dose of 10×10⁶ cells/kg, the KM estimate for RFS at 12 months was 71% (95% CI: 51%, 84%) for arm A, 78% (95% CI: 54%, 90%) for arm B, and 100% (95% CI: 100%, 100%) for arm C (see FIG. 22).

### Clinical Safety

Eighty-one participants received a cellular therapy product of the present disclosure, and 1 additional participant who received cellular material was included in the safety analysis set. Per protocol, participants were followed for safety for 12 months, after which participants were followed in long-term follow-up for OS. The median safety follow-up time was 12.0 (range: 1.0 to 12) months, and the median follow-up time for OS was 12.5 (range: 1.0 to 46.8) months.

The study was designed with dose-escalation and dose-expansion stages based on the Tₘₑₘ component dose. The dose escalation schema is as described herein. In arm A, the target Tₘₑₘ dose was escalated from 3×10⁶ Tₘₑₘ/kg to 100×10⁶ Tₘₑₘ/kg across 4 dose cohorts. In arm B, the target Tₘₑₘ dose was initially 1×10⁶ Tₘₑₘ/kg; however, the first participant treated at this dose experienced graft failure. After this first participant in arm B, the Tₘₑₘ dose level was increased to a target level of 10×10⁶ Tₘₑₘ/kg and then escalated to 100×10⁶ Tₘₑₘ/kg across 3 dose cohorts. No dose-limiting toxicities were observed in participants enrolled on either arm A or arm B. No relationship between Tₘₑₘ dose and risk of acute GVHD (aGVHD) or chronic GVHD (cGVHD) has been observed. Arm C target dose levels were based on safety findings from arm A as described in the study protocol. Enrollment is open on Study OGFT001-001 in arms B and C, with a target Tₘₑₘ dose of 100×10⁶ Tₘₑₘ/kg.

### Adverse Events (AEs)

A summary of treatment-emergent AEs (TEAEs) is provided below in TABLE 17. All AEs herein were treatment emergent and are described as AEs.

Overall, 63 participants (77%) had at least 1 AE, and 42 participants (51%) had an AE that was grade ≥3. Of those participants who had an AE, 33 participants (40%) had an AE that was deemed by the investigator to be related to the cellular therapy, and 17 participants (21%) had a therapy-related AE that was grade ≥3. Twenty-five participants (31%) had a serious AE (SAE), and 9 participants (11%) had an SAE that was related to the cellular therapy. Two participants had AEs that led to study withdrawal, and three participants had AEs leading to death.

In TABLE 17, “AE” refers to adverse event, “CTCAE” refers to Common Terminology Criteria for Adverse Events, “MedDRA” refers to Medical Dictionary for Regulatory Activities, “MMUD” refers to mismatched (7 of 8 alleles) unrelated donor, “MRD” refers to matched related donor, “MUD” refers to matched unrelated donor, and “TEAE” refers to treatment-emergent adverse event. TEAE is depicted as an AE that starts on or after therapy administration through day +100. Any TEAE with missing relationship to study treatment was included as related, any TEAE with missing seriousness was included as serious, and any TEAE with missing grade was included as grade 3 or higher.

TABLE 17

| | Arm A | | | | | |
|---|---|---|---|---|---|---|
| | MRD/MUD | MMUD | Total | Arm B | Arm C | Total |
| | (N = 26) | (N = 17) | (N = 43) | (N = 28) | (N = 11) | (N = 82) |
| | n (%) | n (%) | n (%) | n (%) | n (%) | n (%) |
| Participants with any TEAE | 25 (96.2) | 14 (82.4) | 39 (90.7) | 19 (67.9) | 5 (45.5) | 63 (76.8) |
| Participants with any grade ≥3 TEAE | 13 (50.0) | 11 (64.7) | 24 (55.8) | 16 (57.1) | 2 (18.2) | 42 (51.2) |
| Participants with any therapy-related | 10 (38.5) | 8 (47.1) | 18 (41.9) | 13 (46.4) | 2 (18.2) | 33 (40.2) |
| TEAE | | | | | | |
| Participants with any grade ≥3 | 5 (19.2) | 5 (29.4) | 10 (23.3) | 6 (21.4) | 1 (9.1) | 17 (20.7) |
| therapy-related TEAE | | | | | | |
| Participants with any serious TEAE | 6 (23.1) | 6 (35.3) | 12 (27.9) | 11 (39.3) | 2 (18.2) | 25 (30.5) |
| Participants with any therapy-related | 2 (7.7) | 2 (11.8) | 4 (9.3) | 5 (17.9) | 0 | 9 (11.0) |
| serious TEAE | | | | | | |
| Participants with any TEAE leading to | 0 | 0 | 0 | 0 | 0 | 0 |
| treatment withdrawn | | | | | | |
| Participants with any therapy-related | 0 | 0 | 0 | 0 | 0 | 0 |
| TEAE leading to treatment withdrawn | | | | | | |
| Participants with any TEAE leading to | 0 | 1 (5.9) | 1 (2.3) | 1 (3.6) | 0 | 2 (2.4) |
| study discontinuation | | | | | | |
| Participants with any therapy-related | 0 | 1 (5.9) | 1 (2.3) | 0 | 0 | 1 (1.2) |
| TEAE leading to study | | | | | | |
| discontinuation | | | | | | |
| Participants with any TEAE leading to | 0 | 1 (5.9) | 1 (2.3) | 2 (7.1) | 0 | 3 (3.7) |
| death | | | | | | |
| Participants with any therapy-related | 0 | 0 | 0 | 1 (3.6) | 0 | 1 (1.2) |
| TEAE leading to death | | | | | | |

Overall, the most common AEs by system organ class (SOC) were gastrointestinal disorders (49 participants, 60%), general disorders and administration site conditions (42 participants, 51%), and blood and lymphatic system disorders (41 participants, 50%) (see TABLE 18). AEs occurring in ≥10% of participants overall by preferred term are listed by preferred term frequency and worst grade in TABLE 18.

By preferred term, the most common AEs were diarrhea (29 participants, 35%), febrile neutropenia (26 participants, 32%), and thrombocytopenia (20 participants, 24%).

In TABLE 18, “AE” refers to adverse event, “CTCAE” refers to Common Terminology Criteria for Adverse Events, “MedDRA” refers to Medical Dictionary for Regulatory Activities, “MMUD” refers to mismatched (7 of 8 alleles) unrelated donor, “MRD” refers to matched related donor, “MUD” refers to matched unrelated donor, and “TEAE” refers to treatment-emergent adverse event. TEAE is depicted as an AE that starts on or after therapy administration through day +100. TEAEs with missing grade were also included; the missing grade is presented as “Unknown”.

TABLE 18

| | Arm A | | | | | |
|---|---|---|---|---|---|---|
| | MRD/MUD | MMUD | Total | Arm B | Arm C | Total |
| | (N = 26) | (N = 17) | (N = 43) | (N = 28) | (N = 11) | (N = 82) |
| Preferred Term | n (%) | n (%) | n (%) | n (%) | n (%) | n (%) |
| Participants with any TEAE | 25 (96.2) | 14 (82.4) | 39 (90.7) | 19 (67.9) | 5 (45.5) | 63 (76.8) |
| Grade 1 | 0 | 0 | 0 | 2 (7.1) | 1 (9.1) | 3 (3.7) |
| Grade 2 | 12 (46.2) | 3 (17.6) | 15 (34.9) | 1 (3.6) | 2 (18.2) | 18 (22.0) |
| Grade 3 | 7 (26.9) | 7 (41.2) | 14 (32.6) | 6 (21.4) | 0 | 20 (24.4) |
| Grade 4 | 6 (23.1) | 3 (17.6) | 9 (20.9) | 7 (25.0) | 2 (18.2) | 18 (22.0) |
| Grade 5 | 0 | 1 (5.9) | 1 (2.3) | 2 (7.1) | 0 | 3 (3.7) |
| Unknown | 0 | 0 | 0 | 1 (3.6) | 0 | 1 (1.2) |
| Diarrhoea | 11 (42.3) | 7 (41.2) | 18 (41.9) | 9 (32.1) | 2 (18.2) | 29 (35.4) |
| Grade 1 | 7 (26.9) | 3 (17.6) | 10 (23.3) | 5 (17.9) | 1 (9.1) | 16 (19.5) |
| Grade 2 | 3 (11.5) | 2 (11.8) | 5 (11.6) | 4 (14.3) | 0 | 9 (11.0) |
| Grade 3 | 1 (3.8) | 1 (5.9) | 2 (4.7) | 0 | 1 (9.1) | 3 (3.7) |
| Unknown | 0 | 1 (5.9) | 1 (2.3) | 0 | 0 | 1 (1.2) |
| Febrile neutropenia | 11 (42.3) | 3 (17.6) | 14 (32.6) | 10 (35.7) | 2 (18.2) | 26 (31.7) |
| Grade 1 | 2 (7.7) | 0 | 2 (4.7) | 4 (14.3) | 0 | 6 (7.3) |
| Grade 2 | 3 (11.5) | 0 | 3 (7.0) | 2 (7.1) | 1 (9.1) | 6 (7.3) |
| Grade 3 | 6 (23.1) | 3 (17.6) | 9 (20.9) | 4 (14.3) | 1 (9.1) | 14 (17.1) |
| Thrombocytopenia | 6 (23.1) | 4 (23.5) | 10 (23.3) | 8 (28.6) | 2 (18.2) | 20 (24.4) |
| Grade 1 | 0 | 2 (11.8) | 2 (4.7) | 0 | 0 | 2 (2.4) |
| Grade 2 | 1 (3.8) | 1 (5.9) | 2 (4.7) | 0 | 0 | 2 (2.4) |
| Grade 3 | 0 | 0 | 0 | 2 (7.1) | 0 | 2 (2.4) |
| Grade 4 | 5 (19.2) | 1 (5.9) | 6 (14.0) | 6 (21.4) | 2 (18.2) | 14 (17.1) |
| Anaemia | 7 (26.9) | 2 (11.8) | 9 (20.9) | 8 (28.6) | 2 (18.2) | 19 (23.2) |
| Grade 1 | 1 (3.8) | 1 (5.9) | 2 (4.7) | 2 (7.1) | 0 | 4 (4.9) |
| Grade 2 | 0 | 0 | 0 | 2 (7.1) | 1 (9.1) | 3 (3.7) |
| Grade 3 | 6 (23.1) | 1 (5.9) | 7 (16.3) | 4 (14.3) | 1 (9.1) | 12 (14.6) |
| Nausea | 10 (38.5) | 5 (29.4) | 15 (34.9) | 4 (14.3) | 0 | 19 (23.2) |
| Grade 1 | 8 (30.8) | 2 (11.8) | 10 (23.3) | 2 (7.1) | 0 | 12 (14.6) |
| Grade 2 | 2 (7.7) | 3 (17.6) | 5 (11.6) | 2 (7.1) | 0 | 7 (8.5) |
| Headache | 9 (34.6) | 4 (23.5) | 13 (30.2) | 4 (14.3) | 1 (9.1) | 18 (22.0) |
| Grade 1 | 8 (30.8) | 2 (11.8) | 10 (23.3) | 4 (14.3) | 1 (9.1) | 15 (18.3) |
| Grade 2 | 1 (3.8) | 2 (11.8) | 3 (7.0) | 0 | 0 | 3 (3.7) |
| Mucosal inflammation | 4 (15.4) | 6 (35.3) | 10 (23.3) | 8 (28.6) | 0 | 18 (22.0) |
| Grade 1 | 0 | 1 (5.9) | 1 (2.3) | 3 (10.7) | 0 | 4 (4.9) |
| Grade 2 | 2 (7.7) | 4 (23.5) | 6 (14.0) | 3 (10.7) | 0 | 9 (11.0) |
| Grade 3 | 2 (7.7) | 1 (5.9) | 3 (7.0) | 1 (3.6) | 0 | 4 (4.9) |
| Grade 4 | 0 | 0 | 0 | 1 (3.6) | 0 | 1 (1.2) |
| Acute graft versus host | 8 (30.8) | 4 (23.5) | 12 (27.9) | 4 (14.3) | 1 (9.1) | 17 (20.7) |
| disease | | | | | | |
| Grade 1 | 3 (11.5) | 2 (11.8) | 5 (11.6) | 2 (7.1) | 1 (9.1) | 8 (9.8) |
| Grade 2 | 5 (19.2) | 2 (11.8) | 7 (16.3) | 1 (3.6) | 0 | 8 (9.8) |
| Grade 3 | 0 | 0 | 0 | 1 (3.6) | 0 | 1 (1.2) |
| Stomatitis | 10 (38.5) | 2 (11.8) | 12 (27.9) | 3 (10.7) | 2 (18.2) | 17 (20.7) |
| Grade 1 | 2 (7.7) | 0 | 2 (4.7) | 1 (3.6) | 0 | 3 (3.7) |
| Grade 2 | 6 (23.1) | 1 (5.9) | 7 (16.3) | 1 (3.6) | 2 (18.2) | 10 (12.2) |
| Grade 3 | 2 (7.7) | 1 (5.9) | 3 (7.0) | 1 (3.6) | 0 | 4 (4.9) |
| Hypomagnesaemia | 6 (23.1) | 4 (23.5) | 10 (23.3) | 4 (14.3) | 0 | 14 (17.1) |
| Grade 1 | 3 (11.5) | 2 (11.8) | 5 (11.6) | 2 (7.1) | 0 | 7 (8.5) |
| Grade 2 | 3 (11.5) | 2 (11.8) | 5 (11.6) | 2 (7.1) | 0 | 7 (8.5) |
| Abdominal pain | 6 (23.1) | 3 (17.6) | 9 (20.9) | 4 (14.3) | 0 | 13 (15.9) |
| Grade 1 | 4 (15.4) | 2 (11.8) | 6 (14.0) | 1 (3.6) | 0 | 7 (8.5) |
| Grade 2 | 2 (7.7) | 1 (5.9) | 3 (7.0) | 3 (10.7) | 0 | 6 (7.3) |
| Fatigue | 4 (15.4) | 2 (11.8) | 6 (14.0) | 5 (17.9) | 2 (18.2) | 13 (15.9) |
| Grade 1 | 2 (7.7) | 1 (5.9) | 3 (7.0) | 2 (7.1) | 1 (9.1) | 6 (7.3) |
| Grade 2 | 2 (7.7) | 1 (5.9) | 3 (7.0) | 2 (7.1) | 1 (9.1) | 6 (7.3) |
| Grade 3 | 0 | 0 | 0 | 1 (3.6) | 0 | 1 (1.2) |
| Pyrexia | 5 (19.2) | 2 (11.8) | 7 (16.3) | 5 (17.9) | 1 (9.1) | 13 (15.9) |
| Grade 1 | 5 (19.2) | 2 (11.8) | 7 (16.3) | 3 (10.7) | 1 (9.1) | 11 (13.4) |
| Grade 2 | 0 | 0 | 0 | 1 (3.6) | 0 | 1 (1.2) |
| Grade 3 | 0 | 0 | 0 | 1 (3.6) | 0 | 1 (1.2) |
| Vomiting | 7 (26.9) | 3 (17.6) | 10 (23.3) | 3 (10.7) | 0 | 13 (15.9) |
| Grade 1 | 6 (23.1) | 2 (11.8) | 8 (18.6) | 1 (3.6) | 0 | 9 (11.0) |
| Grade 2 | 1 (3.8) | 1 (5.9) | 2 (4.7) | 2 (7.1) | 0 | 4 (4.9) |
| Leukopenia | 4 (15.4) | 1 (5.9) | 5 (11.6) | 6 (21.4) | 1 (9.1) | 12 (14.6) |
| Grade 1 | 0 | 0 | 0 | 1 (3.6) | 0 | 1 (1.2) |
| Grade 2 | 0 | 0 | 0 | 0 | 0 | 0 |
| Grade 3 | 1 (3.8) | 1 (5.9) | 2 (4.7) | 0 | 0 | 2 (2.4) |
| Grade 4 | 3 (11.5) | 0 | 3 (7.0) | 5 (17.9) | 1 (9.1) | 9 (11.0) |
| Rash | 7 (26.9) | 2 (11.8) | 9 (20.9) | 2 (7.1) | 1 (9.1) | 12 (14.6) |
| Grade 1 | 6 (23.1) | 0 | 6 (14.0) | 2 (7.1) | 0 | 8 (9.8) |
| Grade 2 | 1 (3.8) | 1 (5.9) | 2 (4.7) | 0 | 0 | 2 (2.4) |
| Grade 3 | 0 | 1 (5.9) | 1 (2.3) | 0 | 1 (9.1) | 2 (2.4) |
| Decreased appetite | 6 (23.1) | 0 | 6 (14.0) | 5 (17.9) | 0 | 11 (13.4) |
| Grade 1 | 4 (15.4) | 0 | 4 (9.3) | 4 (14.3) | 0 | 8 (9.8) |
| Grade 2 | 1 (3.8) | 0 | 1 (2.3) | 1 (3.6) | 0 | 2 (2.4) |
| Grade 3 | 1 (3.8) | 0 | 1 (2.3) | 0 | 0 | 1 (1.2) |
| Pruritus | 7 (26.9) | 2 (11.8) | 9 (20.9) | 0 | 1 (9.1) | 10 (12.2) |
| Grade 1 | 7 (26.9) | 2 (11.8) | 9 (20.9) | 0 | 1 (9.1) | 10 (12.2) |
| Insomnia | 6 (23.1) | 0 | 6 (14.0) | 3 (10.7) | 0 | 9 (11.0) |
| Grade 1 | 6 (23.1) | 0 | 6 (14.0) | 2 (7.1) | 0 | 8 (9.8) |
| Grade 2 | 0 | 0 | 0 | 1 (3.6) | 0 | 1 (1.2) |

Grade ≥3 AEs occurring in 5 participants by SOC are listed by SOC, preferred term, worst grade, and study arm in TABLE 19. As shown in TABLE 19, the most common grade ≥3 AEs by SOC were blood and lymphatic system disorders (32 participants, 39%), infections and infestations (11 participants, 13%), and gastrointestinal disorders (10 participants, 12%). By preferred term, the most common grade ≥3 AEs were thrombocytopenia (16 participants, 20%), febrile neutropenia (14 participants, 17%), and anemia (12 participants, 15%) (see TABLE 19).

The most common grade ≥3 AEs deemed by the investigator to be related to the therapy by SOC were blood and lymphatic disorders (5 participants, 6%); injury, poisoning, and procedural complications (5 participants, 6%), and infections and infestations (4 participants, 5%) (see TABLE 19).

In TABLE 19, “AE” refers to adverse event, “CTCAE” refers to Common Terminology Criteria for Adverse Events, “MedDRA” refers to Medical Dictionary for Regulatory Activities, “MMUD” refers to mismatched (7 of 8 alleles) unrelated donor, “MRD” refers to matched related donor, “MUD” refers to matched unrelated donor, and “TEAE” refers to treatment-emergent adverse event. TEAE is depicted as an AE that starts on or after therapy administration through day +100. Participants may have had >1 AE per SOC and/or preferred term. At each level of summarization, a participant was counted only once under the most severe event. TEAEs with missing grade were also included; the missing grade is presented as “Unknown”.

TABLE 19

| | Arm A | | | | | |
|---|---|---|---|---|---|---|
| | MRD/MUD | MMUD | Total | Arm B | Arm C | Total |
| System Organ Class | (N = 26) | (N = 17) | (N = 43) | (N = 28) | (N = 11) | (N = 82) |
| Preferred Term | n (%) | n (%) | n (%) | n (%) | n (%) | n (%) |
| Participants with any | 13 (50.0) | 11 (64.7) | 24 (55.8) | 16 (57.1) | 2 (18.2) | 42 (51.2) |
| grade ≥3 TEAE | | | | | | |
| Grade 3 | 7 (26.9) | 7 (41.2) | 14 (32.6) | 6 (21.4) | 0 | 20 (24.4) |
| Grade 4 | 6 (23.1) | 3 (17.6) | 9 (20.9) | 7 (25.0) | 2 (18.2) | 18 (22.0) |
| Grade 5 | 0 | 1 (5.9) | 1 (2.3) | 2 (7.1) | 0 | 3 (3.7) |
| Unknown | 0 | 0 | 0 | 1 (3.6) | 0 | 1 (1.2) |
| Blood and lymphatic | 10 (38.5) | 7 (41.2) | 17 (39.5) | 13 (46.4) | 2 (18.2) | 32 (39.0) |
| system disorders | | | | | | |
| Grade 3 | 4 (15.4) | 6 (35.3) | 10 (23.3) | 7 (25.0) | 0 | 17 (20.7) |
| Grade 4 | 6 (23.1) | 1 (5.9) | 7 (16.3) | 6 (21.4) | 2 (18.2) | 15 (18.3) |
| Thrombocytopenia | 5 (19.2) | 1 (5.9) | 6 (14.0) | 8 (28.6) | 2 (18.2) | 16 (19.5) |
| Grade 3 | 0 | 0 | 0 | 2 (7.1) | 0 | 2 (2.4) |
| Grade 4 | 5 (19.2) | 1 (5.9) | 6 (14.0) | 6 (21.4) | 2 (18.2) | 14 (17.1) |
| Febrile neutropenia | 6 (23.1) | 3 (17.6) | 9 (20.9) | 4 (14.3) | 1 (9.1) | 14 (17.1) |
| Grade 3 | 6 (23.1) | 3 (17.6) | 9 (20.9) | 4 (14.3) | 1 (9.1) | 14 (17.1) |
| Anaemia | 6 (23.1) | 1 (5.9) | 7 (16.3) | 4 (14.3) | 1 (9.1) | 12 (14.6) |
| Grade 3 | 6 (23.1) | 1 (5.9) | 7 (16.3) | 4 (14.3) | 1 (9.1) | 12 (14.6) |
| Leukopenia | 4 (15.4) | 1 (5.9) | 5 (11.6) | 5 (17.9) | 1 (9.1) | 11 (13.4) |
| Grade 3 | 1 (3.8) | 1 (5.9) | 2 (4.7) | 0 | 0 | 2 (2.4) |
| Grade 4 | 3 (11.5) | 0 | 3 (7.0) | 5 (17.9) | 1 (9.1) | 9 (11.0) |
| Neutropenia | 2 (7.7) | 0 | 2 (4.7) | 2 (7.1) | 1 (9.1) | 5 (6.1) |
| Grade 4 | 2 (7.7) | 0 | 2 (4.7) | 2 (7.1) | 1 (9.1) | 5 (6.1) |
| Lymphopenia | 2 (7.7) | 0 | 2 (4.7) | 1 (3.6) | 1 (9.1) | 4 (4.9) |
| Grade 3 | 0 | 0 | 0 | 0 | 1 (9.1) | 1 (1.2) |
| Grade 4 | 2 (7.7) | 0 | 2 (4.7) | 1 (3.6) | 0 | 3 (3.7) |
| Haemolytic anaemia | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Leukocytosis | 0 | 0 | 0 | 1 (3.6) | 0 | 1 (1.2) |
| Grade 3 | 0 | 0 | 0 | 1 (3.6) | 0 | 1 (1.2) |
| Infections and infestations | 4 (15.4) | 2 (11.8) | 6 (14.0) | 5 (17.9) | 0 | 11 (13.4) |
| Grade 3 | 4 (15.4) | 2 (11.8) | 6 (14.0) | 3 (10.7) | 0 | 9 (11.0) |
| Grade 4 | 0 | 0 | 0 | 1 (3.6) | 0 | 1 (1.2) |
| Grade 5 | 0 | 0 | 0 | 1 (3.6) | 0 | 1 (1.2) |
| Sepsis | 1 (3.8) | 1 (5.9) | 2 (4.7) | 1 (3.6) | 0 | 3 (3.7) |
| Grade 3 | 1 (3.8) | 1 (5.9) | 2 (4.7) | 1 (3.6) | 0 | 3 (3.7) |
| Clostridium difficile infection | 1 (3.8) | 0 | 1 (2.3) | 1 (3.6) | 0 | 2 (2.4) |
| Grade 3 | 1 (3.8) | 0 | 1 (2.3) | 1 (3.6) | 0 | 2 (2.4) |
| Bronchopulmonary aspergillosis | 0 | 0 | 0 | 1 (3.6) | 0 | 1 (1.2) |
| Grade 5 | 0 | 0 | 0 | 1 (3.6) | 0 | 1 (1.2) |
| Cytomegalovirus infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Cytomegalovirus viraemia | 0 | 0 | 0 | 1 (3.6) | 0 | 1 (1.2) |
| Grade 3 | 0 | 0 | 0 | 1 (3.6) | 0 | 1 (1.2) |
| Encephalitis | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Escherichia bacteraemia | 0 | 0 | 0 | 1 (3.6) | 0 | 1 (1.2) |
| Grade 3 | 0 | 0 | 0 | 1 (3.6) | 0 | 1 (1.2) |
| Klebsiella sepsis | 0 | 0 | 0 | 1 (3.6) | 0 | 1 (1.2) |
| Grade 4 | 0 | 0 | 0 | 1 (3.6) | 0 | 1 (1.2) |
| Pseudomonas infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Serratia sepsis | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Staphylococcal infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Staphylococcal sepsis | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Gastrointestinal disorders | 3 (11.5) | 4 (23.5) | 7 (16.3) | 2 (7.1) | 1 (9.1) | 10 (12.2) |
| Grade 3 | 3 (11.5) | 3 (17.6) | 6 (14.0) | 2 (7.1) | 1 (9.1) | 9 (11.0) |
| Unknown | 0 | 1 (5.9) | 1 (2.3) | 0 | 0 | 1 (1.2) |
| Diarrhoea | 1 (3.8) | 2 (11.8) | 3 (7.0) | 0 | 1 (9.1) | 4 (4.9) |
| Grade 3 | 1 (3.8) | 1 (5.9) | 2 (4.7) | 0 | 1 (9.1) | 3 (3.7) |
| Unknown | 0 | 1 (5.9) | 1 (2.3) | 0 | 0 | 1 (1.2) |
| Stomatitis | 2 (7.7) | 1 (5.9) | 3 (7.0) | 1 (3.6) | 0 | 4 (4.9) |
| Grade 3 | 2 (7.7) | 1 (5.9) | 3 (7.0) | 1 (3.6) | 0 | 4 (4.9) |
| Colitis | 0 | 0 | 0 | 1 (3.6) | 0 | 1 (1.2) |
| Grade 3 | 0 | 0 | 0 | 1 (3.6) | 0 | 1 (1.2) |
| Oral pain | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| General disorders and | 2 (7.7) | 2 (11.8) | 4 (9.3) | 4 (14.3) | 0 | 8 (9.8) |
| administration site conditions | | | | | | |
| Grade 3 | 2 (7.7) | 1 (5.9) | 3 (7.0) | 3 (10.7) | 0 | 6 (7.3) |
| Grade 4 | 0 | 0 | 0 | 1 (3.6) | 0 | 1 (1.2) |
| Grade 5 | 0 | 1 (5.9) | 1 (2.3) | 0 | 0 | 1 (1.2) |
| Mucosal inflammation | 2 (7.7) | 1 (5.9) | 3 (7.0) | 2 (7.1) | 0 | 5 (6.1) |
| Grade 3 | 2 (7.7) | 1 (5.9) | 3 (7.0) | 1 (3.6) | 0 | 4 (4.9) |
| Grade 4 | 0 | 0 | 0 | 1 (3.6) | 0 | 1 (1.2) |
| Fatigue | 0 | 0 | 0 | 1 (3.6) | 0 | 1 (1.2) |
| Grade 3 | 0 | 0 | 0 | 1 (3.6) | 0 | 1 (1.2) |
| Multiple organ | 0 | 1 (5.9) | 1 (2.3) | 0 | 0 | 1 (1.2) |
| dysfunction syndrome | 0 | 1 (5.9) | 1 (2.3) | 0 | 0 | 1 (1.2) |
| Grade 5 | | | | | | |
| Oedema peripheral | 0 | 0 | 0 | 1 (3.6) | 0 | 1 (1.2) |
| Grade 3 | 0 | 0 | 0 | 1 (3.6) | 0 | 1 (1.2) |
| Pyrexia | 0 | 0 | 0 | 1 (3.6) | 0 | 1 (1.2) |
| Grade 3 | 0 | 0 | 0 | 1 (3.6) | 0 | 1 (1.2) |
| Respiratory, thoracic and | 0 | 3 (17.6) | 3 (7.0) | 4 (14.3) | 0 | 7 (8.5) |
| mediastinal disorders | | | | | | |
| Grade 3 | 0 | 2 (11.8) | 2 (4.7) | 2 (7.1) | 0 | 4 (4.9) |
| Grade 4 | 0 | 1 (5.9) | 1 (2.3) | 0 | 0 | 1 (1.2) |
| Grade 5 | 0 | 0 | 0 | 1 (3.6) | 0 | 1 (1.2) |
| Unknown | 0 | 0 | 0 | 1 (3.6) | 0 | 1 (1.2) |
| Dyspnoea | 0 | 1 (5.9) | 1 (2.3) | 1 (3.6) | 0 | 2 (2.4) |
| Grade 3 | 0 | 1 (5.9) | 1 (2.3) | 1 (3.6) | 0 | 2 (2.4) |
| Acute respiratory failure | 0 | 0 | 0 | 1 (3.6) | 0 | 1 (1.2) |
| Grade 5 | 0 | 0 | 0 | 1 (3.6) | 0 | 1 (1.2) |
| Pulmonary alveolar haemorrhage | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Pulmonary embolism | 0 | 0 | 0 | 1 (3.6) | 0 | 1 (1.2) |
| Grade 3 | 0 | 0 | 0 | 1 (3.6) | 0 | 1 (1.2) |
| Respiratory failure | 0 | 1 (5.9) | 1 (2.3) | 0 | 0 | 1 (1.2) |
| Grade 4 | 0 | 1 (5.9) | 1 (2.3) | 0 | 0 | 1 (1.2) |
| Tonsillolith | 0 | 0 | 0 | 1 (3.6) | 0 | 1 (1.2) |
| Unknown | 0 | 0 | 0 | 1 (3.6) | 0 | 1 (1.2) |
| Injury, poisoning and | 1 (3.8) | 2 (11.8) | 3 (7.0) | 3 (10.7) | 0 | 6 (7.3) |
| procedural complications | | | | | | |
| Grade 3 | 1 (3.8) | 0 | 1 (2.3) | 0 | 0 | 1 (1.2) |
| Grade 4 | 0 | 2 (11.8) | 2 (4.7) | 3 (10.7) | 0 | 5 (6.1) |
| Transplant failure | 1 (3.8) | 1 (5.9) | 2 (4.7) | 3 (10.7) | 0 | 5 (6.1) |
| Grade 3 | 1 (3.8) | 0 | 1 (2.3) | 0 | 0 | 1 (1.2) |
| Grade 4 | 0 | 1 (5.9) | 1 (2.3) | 3 (10.7) | 0 | 4 (4.9) |
| Transfusion-related | 0 | 1 (5.9) | 1 (2.3) | 0 | 0 | 1 (1.2) |
| circulatory overload | | | | | | |
| Grade 4 | 0 | 1 (5.9) | 1 (2.3) | 0 | 0 | 1 (1.2) |

Overall, 16 participants died: 8 participants due to disease progression, 7 participants due to AEs, and 1 participant due to “other” reasons. Of the 7 deaths due to AEs, 3 participants had AEs leading to death that were treatment emergent (i.e., occurred between therapy administration and day +100): 1 participant in arm A with multiple organ dysfunction syndrome on day +32 that led to death on day +68, 1 participant in arm B with acute respiratory failure on day +56 that led to death on day +74, and 1 participant in arm B with transplant failure on day +43 and bronchopulmonary aspergillosis on day +52 that led to death on day +76. The grade 4 AE of transplant failure also led to the participant's withdrawal from study and was the only AE leading to death that was deemed related to the cellular therapy; the ensuing grade 5 bronchopulmonary aspergillosis was not related to the cellular therapy. The grade 5 AE of acute respiratory failure was the only other AE that led to withdrawal from study. Deaths due to AEs that were not treatment emergent were attributed to sepsis on day +228 in 1 participant in arm A (died on day +232), coronavirus infection on day +184 in 1 participant in arm A (died on day +213), hemorrhagic shock on day +210 in 1 participant in arm B (died on day +218), and viral pneumonia (COVID-19) and hypoxia on day −4 in 1 participant in arm B (died on day +111). The 1 participant in arm A with a cause of death listed as “other” completed the study and died on day +661 due to “significant decline at home with malnutrition.” Other Serious Adverse Events (SAEs)

SAEs are listed by preferred term frequency and worst grade in TABLE 20. The most common SAEs were transplant failure (5 participants, 6%); aGVHD (3 participants, 4%); and acute kidney injury, febrile neutropenia, pyrexia, sepsis, and thrombocytopenia (2 participants [2%] each) (see TABLE 20). As shown in TABLE 20, all other SAEs occurred in 1 participant each.

In TABLE 20, “AE” refers to adverse event, “CTCAE” refers to Common Terminology Criteria for Adverse Events, “MedDRA” refers to Medical Dictionary for Regulatory Activities, “MMUD” refers to mismatched (7 of 8 alleles) unrelated donor, “MRD” refers to matched related donor, “MUD” refers to matched unrelated donor, and “TEAE” refers to treatment-emergent adverse event. TEAE is depicted as an AE that starts on or after therapy administration through day +100. TEAEs with missing seriousness were also included. TEAEs with missing grade were also included; the missing grade is presented as “Unknown”.

TABLE 20

| | Arm A | | | | | |
|---|---|---|---|---|---|---|
| | MRD/MUD | MMUD | Total | Arm B | Arm C | Total |
| | (N = 26) | (N = 17) | (N = 43) | (N = 28) | (N = 11) | (N = 82) |
| Preferred Term | n (%) | n (%) | n (%) | n (%) | n (%) | n (%) |
| Participants with any serious TEAE | 6 (23.1) | 6 (35.3) | 12 (27.9) | 11 (39.3) | 2 (18.2) | 25 (30.5) |
| Grade 1 | 0 | 0 | 0 | 1 (3.6) | 1 (9.1) | 2 (2.4) |
| Grade 2 | 2 (7.7) | 0 | 2 (4.7) | 0 | 1 (9.1) | 3 (3.7) |
| Grade 3 | 4 (15.4) | 2 (11.8) | 6 (14.0) | 4 (14.3) | 0 | 10 (12.2) |
| Grade 4 | 0 | 3 (17.6) | 3 (7.0) | 3 (10.7) | 0 | 6 (7.3) |
| Grade 5 | 0 | 1 (5.9) | 1 (2.3) | 2 (7.1) | 0 | 3 (3.7) |
| Unknown | 0 | 0 | 0 | 1 (3.6) | 0 | 1 (1.2) |
| Transplant failure | 1 (3.8) | 1 (5.9) | 2 (4.7) | 3 (10.7) | 0 | 5 (6.1) |
| Grade 3 | 1 (3.8) | 0 | 1 (2.3) | 0 | 0 | 1 (1.2) |
| Grade 4 | 0 | 1 (5.9) | 1 (2.3) | 3 (10.7) | 0 | 4 (4.9) |
| Acute graft versus host disease | 2 (7.7) | 0 | 2 (4.7) | 1 (3.6) | 0 | 3 (3.7) |
| Grade 2 | 2 (7.7) | 0 | 2 (4.7) | 0 | 0 | 2 (2.4) |
| Grade 3 | 0 | 0 | 0 | 1 (3.6) | 0 | 1 (1.2) |
| Acute kidney injury | 0 | 0 | 0 | 2 (7.1) | 0 | 2 (2.4) |
| Grade 3 | 0 | 0 | 0 | 1 (3.6) | 0 | 1 (1.2) |
| Grade 4 | 0 | 0 | 0 | 1 (3.6) | 0 | 1 (1.2) |
| Febrile neutropenia | 1 (3.8) | 1 (5.9) | 2 (4.7) | 0 | 0 | 2 (2.4) |
| Grade 3 | 1 (3.8) | 1 (5.9) | 2 (4.7) | 0 | 0 | 2 (2.4) |
| Pyrexia | 0 | 0 | 0 | 1 (3.6) | 1 (9.1) | 2 (2.4) |
| Grade 1 | 0 | 0 | 0 | 1 (3.6) | 1 (9.1) | 2 (2.4) |
| Sepsis | 1 (3.8) | 0 | 1 (2.3) | 1 (3.6) | 0 | 2 (2.4) |
| Grade 3 | 1 (3.8) | 0 | 1 (2.3) | 1 (3.6) | 0 | 2 (2.4) |
| Thrombocytopenia | 0 | 1 (5.9) | 1 (2.3) | 1 (3.6) | 0 | 2 (2.4) |
| Grade 3 | 0 | 0 | 0 | 1 (3.6) | 0 | 1 (1.2) |
| Grade 4 | 0 | 1 (5.9) | 1 (2.3) | 0 | 0 | 1 (1.2) |
| Acute respiratory failure | 0 | 0 | 0 | 1 (3.6) | 0 | 1 (1.2) |
| Grade 5 | 0 | 0 | 0 | 1 (3.6) | 0 | 1 (1.2) |
| Bacterial infection | 0 | 0 | 0 | 0 | 1 (9.1) | 1 (1.2) |
| Grade 2 | 0 | 0 | 0 | 0 | 1 (9.1) | 1 (1.2) |
| Bronchopulmonary aspergillosis | 0 | 0 | 0 | 1 (3.6) | 0 | 1 (1.2) |
| Grade 5 | 0 | 0 | 0 | 1 (3.6) | 0 | 1 (1.2) |
| Clostridium difficile infection | 0 | 0 | 0 | 1 (3.6) | 0 | 1 (1.2) |
| Grade 3 | 0 | 0 | 0 | 1 (3.6) | 0 | 1 (1.2) |
| Cytomegalovirus infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Cytomegalovirus viraemia | 0 | 0 | 0 | 1 (3.6) | 0 | 1 (1.2) |
| Grade 3 | 0 | 0 | 0 | 1 (3.6) | 0 | 1 (1.2) |
| Dizziness | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Dyspnoea | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Encephalitis | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Escherichia bacteraemia | 0 | 0 | 0 | 1 (3.6) | 0 | 1 (1.2) |
| Grade 3 | 0 | 0 | 0 | 1 (3.6) | 0 | 1 (1.2) |
| Haemolytic anaemia | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Klebsiella sepsis | 0 | 0 | 0 | 1 (3.6) | 0 | 1 (1.2) |
| Grade 4 | 0 | 0 | 0 | 1 (3.6) | 0 | 1 (1.2) |
| Leukaemia recurrent | 0 | 1 (5.9) | 1 (2.3) | 0 | 0 | 1 (1.2) |
| Grade 4 | 0 | 1 (5.9) | 1 (2.3) | 0 | 0 | 1 (1.2) |
| Multiple organ dysfunction syndrome | 0 | 1 (5.9) | 1 (2.3) | 0 | 0 | 1 (1.2) |
| Grade 5 | 0 | 1 (5.9) | 1 (2.3) | 0 | 0 | 1 (1.2) |
| Oedema peripheral | 0 | 0 | 0 | 1 (3.6) | 0 | 1 (1.2) |
| Grade 3 | 0 | 0 | 0 | 1 (3.6) | 0 | 1 (1.2) |
| Oral pain | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Post transplant lymphoproliferative | 0 | 0 | 0 | 1 (3.6) | 0 | 1 (1.2) |
| disorder | | | | | | |
| Unknown | 0 | 0 | 0 | 1 (3.6) | 0 | 1 (1.2) |
| Pseudomonas infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Pulmonary alveolar haemorrhage | 0 | 1 (5.9) | 1 (2.3) | 0 | 0 | 1 (1.2) |
| Grade 3 | 0 | 1 (5.9) | 1 (2.3) | 0 | 0 | 1 (1.2) |
| Pulmonary embolism | 0 | 0 | 0 | 1 (3.6) | 0 | 1 (1.2) |
| Grade 3 | 0 | 0 | 0 | 1 (3.6) | 0 | 1 (1.2) |
| Serratia sepsis | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Staphylococcal infection | 1 (3.8) | 0 | 1 (2.3) | 0 | 0 | 1 (1.2) |
| Grade 3 | 1 (3.8) | 0 | 1 (2.3) | 0 | 0 | 1 (1.2) |
| Transfusion-related circulatory | 0 | 1 (5.9) | 1 (2.3) | 0 | 0 | 1 (1.2) |
| overload | | | | | | |
| Grade 4 | 0 | 1 (5.9) | 1 (2.3) | 0 | 0 | 1 (1.2) |

Two participants had AEs that were treatment emergent (i.e., occurred between therapy administration and day +100) and led to withdrawal from study. One participant in arm A had a grade 4 SAE of transplant failure (secondary graft failure) on day +54 that was reported as related to the cellular therapy. One participant in arm B died of a grade 5 AE of acute respiratory failure that was considered not related to the cellular therapy. Additionally, 2 participants had AEs that were not treatment emergent but led to withdrawal from study: 1 participant in arm A had an SAE of coronavirus infection on day +184 that led to the participant's death on day +213, and 1 participant in arm B had a grade 4 SAE of hypoxia on day −4. Neither of these events were considered related to the cellular therapy.

AEs of special interest for the cellular therapy include aGVHD, cGVHD, posttransplant lymphoproliferative disorder (PTLD), and grade ³2 infections. AEs of special interest are summarized in TABLE 21.

In TABLE 21, “AESI” refers to adverse event of special interest, “aGVHD” refers to acute graft-versus-host disease, “cGVHD” refers to chronic graft-versus-host disease, “MedDRA” refers to Medical Dictionary for Regulatory Activities, “MMUD” refers to mismatched (7 of 8 alleles) unrelated donor, “MRD” refers to matched related donor, “MUD” refers to matched unrelated donor, and “PTLD” refers to post-transplant lymphoproliferative disorder. All AEs included were TEAEs that started on or after therapy administration through day +100. Any TEAE with missing relationship to study treatment was included as related, any TEAE with missing seriousness was included as serious, and any TEAE with missing grade was included as grade ≥3.

TABLE 21

| | Arm A | | | | | |
|---|---|---|---|---|---|---|
| | MRD/MUD | MMUD | Total | Arm B | Arm C | Total |
| | (N = 26) | (N = 17) | (N = 43) | (N = 28) | (N = 11) | (N = 82) |
| | n (%) | n (%) | n (%) | n (%) | n (%) | n (%) |
| Participants with any AESI | 14 (53.8) | 5 (29.4) | 19 (44.2) | 10 (35.7) | 3 (27.3) | 32 (39.0) |
| aGVHD | 8 (30.8) | 4 (23.5) | 12 (27.9) | 5 (17.9) | 1 (9.1) | 18 (22.0) |
| cGVHD | 0 | 0 | 0 | 1 (3.6) | 0 | 1 (1.2) |
| PTLD | 0 | 0 | 0 | 1 (3.6) | 0 | 1 (1.2) |
| Grade ≥2 infections | 8 (30.8) | 4 (23.5) | 12 (27.9) | 8 (28.6) | 2 (18.2) | 22 (26.8) |
| Participants with any Grade ≥3 AESI | 4 (15.4) | 2 (11.8) | 6 (14.0) | 6 (21.4) | 0 | 12 (14.6) |
| Participants with any therapy-related | 6 (23.1) | 3 (17.6) | 9 (20.9) | 9 (32.1) | 1 (9.1) | 19 (23.2) |
| AESI | | | | | | |
| Participants with any Grade ≥3 | 2 (7.7) | 0 | 2 (4.7) | 3 (10.7) | 0 | 5 (6.1) |
| therapy-related AESI | | | | | | |
| Participants with any serious AESI | 6 (23.1) | 1 (5.9) | 7 (16.3) | 6 (21.4) | 1 (9.1) | 14 (17.1) |
| Participants with any therapy-related | 2 (7.7) | 0 | 2 (4.7) | 3 (10.7) | 0 | 5 (6.1) |
| serious AESI | | | | | | |

Acute GVHD (aGVHD)

GVHD is a known complication of HCT. All staging and grading of aGVHD was performed according to the Mount Sinai Acute GVHD International Consortium (MAGIC) Standardization (Harris et al. 2016).

Overall, 18 participants (22%) had aGVHD, mostly grade 1 or 2. One participant in arm B had grade 3 aGVHD of the liver that began on day +51 and resolved on day +71; no participant had grade 4 or 5 aGVHD (see TABLE 21). Three of the 18 participants had serious aGVHD. As of the data cutoff date, aGVHD had resolved in 16 participants and was ongoing in 2 participants. The median time to onset of aGVHD was 31.5 days (range: 19 to 75 days), and the median time to resolution for those whose aGVHD resolved was 23 days (range: 4 to 411 days).

Chronic GVHD (cGVHD)

GVHD is a known complication of HCT. cGVHD was graded per the International National Institutes of Health (NIH) Chronic GVHD Diagnosis and Staging Consensus Working Group Criteria (Jagasia et al. 2015).

One participant in arm B had grade 1, nonserious, oral cGVHD that began on day +97 and was resolved on day +191 (see TABLE 21).

### Post-Transplant Lymphoproliferative Disorder (PTLD)

PTLD is defined as a lymphoma that occurs after HCT or solid organ transplantation. It is a known complication of HCT.

As of the data cutoff date (24 Mar. 2023), 1 participant in arm B had serious, grade 3 polyclonal PTLD that was treatment emergent (began on day +74) and was reported to be resolved (see TABLE 21). One participant in arm A had nonserious Epstein-Barr virus (EBV)-positive PTLD that was not treatment emergent (began on day +173) and was not resolved as of the data cutoff date.

### Grade ≥2 Infections

AlloHCT serves to reconstitute the immune system with healthy cells following MAC depletion of diseased B cells. Opportunistic infections are therefore a known complication of MAC-alloHCT.

Twenty-two participants (27%) had grade ≥2 infections, and these events were serious in 11 participants (13%) (see TABLE 21). Eleven participants (13%) had grade 2 infections, and 9 participants (11%) had grade 3 infections. One participant in arm B had grade ≥4 Klebsiella sepsis, and 1 participant in arm B had grade 5 bronchopulmonary aspergillosis. Grade ≥2 infections had resolved in 18 participants and were ongoing as of the data cutoff date or the date of death in 4 participants. The median time to onset of grade ≥2 infections was 14.5 days (range: 2 to 92 days), and the median time to resolution for those whose infections resolved was 12 days (range: 2 to 385 days).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### Example 4: Additional Arm B Clinical Results

The protocol, materials, and methods described in Example 1 were used to obtain the results described herein in Example 4.

### Background

Allogeneic stem cell transplantation (SCT) can be curative for several high-risk hematologic malignancies, but access was previously limited to patients with a fully matched donor. The introduction of post-transplant cyclophosphamide (PTCy) GvHD prophylaxis has increased the use of haploidentical donors (haplo) for patients who lack an HLA-matched donor. However, clinical outcomes associated with PTCy-based regimens remain challenging due to increased relapse rates (particularly in the setting of reduced intensity conditioning) and the adverse event profile including higher incidence of mucositis, cytokine release syndrome, delayed engraftment and T cell reconstitution, infections, early cardiotoxicity events and death from organ failure (Dulery 2021, Abboud 2021, Nagler 2022, Hoover 2022, Bolaios-Meade 2023). Additionally, the incidence of chronic GvHD after haplo SCT with PTCy is 24-33% at 1 year in various historical cohorts.

The precision engineered cellular therapy product of the present disclosure represents an alternative strategy which may be administered with single agent prophylaxis and does not require PTCy.

### Results

Adult patients with high-risk hematologic malignancies eligible for myeloablative conditioning stem cell transplant (MAC SCT) were enrolled on Arm B (the haplo donor dose expansion arm) of the multicenter phase 1 study of described in Example 1. Haplo was defined as ≥4/8 but <7/8 matched related donor at HLA-A, -B, -C, and -DRB1 typed using DNA-based high-resolution. Patients received MAC conditioning and single agent GVHD prophylaxis with tacrolimus (starting on day −1 and taper day +60).

A total of 34 patients (21 AML, 10 ALL, 1 high/very-high risk MDS, 2 CML) were included. Median age was 42 (range, 21-63) years, 74% were male. Median follow-up was 362 (range, 73-1384) days. Sixteen patients received TBI-based MAC; 18 received busulfan-based MAC (Table). All patients engrafted with median time of 12.0 (range, 8-25) and 15.5 (range, 8-79) days for neutrophil and platelet engraftment, respectively. Two patients had secondary graft failure. Grade 2+aGvHD through Day 180+ occurred in 15% of patients. Grade 3-4 acute GvHD (aGvHD) was rare with only 1 event of grade 3 aGvHD and no grade 4 aGvHD. No patients had moderate-to-severe cGvHD. Estimated incidence of CTCAE grade 2 and ≥grade 3 infections were 9% and 15% at 1 year, respectively. 5 patients died (2 from relapsed disease) during the study period. NRM was 9% at 1 year.

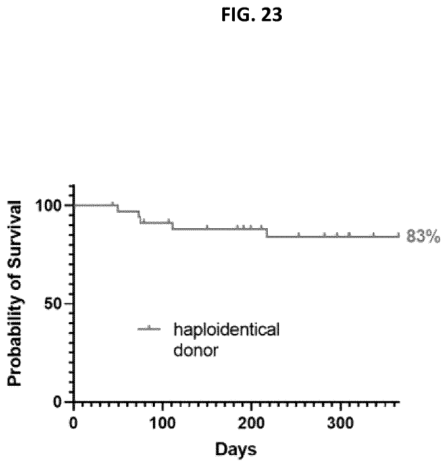

As shown in FIG. 23, the GvHD-and-relapse free survival (GRFS) at 1 year was 83%.

These findings indicate positive safety and efficacy outcomes using the cellular therapy product of the present disclosure for haplo-SCT despite the use of MAC with only single-agent tacrolimus, and without PTCy. With median follow-up of approximately 1 year, no patients experienced moderate or severe cGvHD, the low AE profile in the haplo setting remains favorable, and 1 year GRFS of 83% is very encouraging.

### Example 5: Multiple Sclerosis Clinical Study

### Background

Multiple sclerosis (MS) is an inflammatory disease of the central nervous system that primarily affects young adults, and has an autoimmune pathogenesis. It is the third leading cause of significant disability (after trauma and arthritis) in the 20- to 50-year age range. Since it affects people in their early working careers, it likely accounts for more disability, medical costs and lost income than any other disease of young adults in North America (Auty A, et al. Can J Neurol Sci. 1998; 25:23-30; Whetten-Goldstein K, et al. Mult Scler J. 1998; 4(5):419-425).

There are four types of MS, based on clinical presentation (Auty A et al. Can J Neurol Sci. 1998; 25:23-30): Relapsing/Remitting MS (RRMS), Primary Progressive MS (PPMS), Secondary Progressive MS (SPMS), and Progressive Relapsing MS (PRMS). In RRMS, symptoms occur in a series of attacks characterized by deterioration over a few days of neurological function. Over weeks or months, symptoms resulting from an attack may improve, though recovery may be only partial. Disability may develop by the accumulation of damage despite partial recoveries. About 80% to 90% of patients begin with relapsing/remitting disease. Other patients have symptoms that slowly worsen over months in the absence of acute attacks. This form of MS is termed progressive and is divided into two subtypes (Lublin F D, et al., Neurology. 1996; 46(4):907). Patients who have progressive MS from the onset have PPMS. Patients with PPMS, by definition, have never had even a single acute attack. Patients who begin with relapsing/remitting disease and then convert to the progressive form have SPMS. Attacks are always present at the onset of SPMS and may or may not continue during the progressive phase of the disease. In PRMS, patients start out with a slowly progressive course, punctuated at a later date by acute attacks. The McDonald criteria for the diagnosis of MS recognize PPMS and has formalized the diagnostic requirements for this form of the disease (McDonald W I, et al. Ann Neurol. 2001; 50(1):121-127).

In its more severe forms, severe disability can result from neurological injury, and life expectancy may be shortened. PPMS patients present with loss of neurological function but without relapses. There had been no effective therapy for PPMS but in a randomized clinical trial, it was demonstrated that ocrelizumab slowed progression of disease (Montalban X, et al. N Engl J Med. 2017; 376(3):209-220). Although significant, the improvement in outcome was modest and better treatments are necessary.

Studies of experimental allergic encephalomyelitis (EAE), a murine model for MS, have indicated that disease control can be obtained by conditioning followed by hematopoietic cell transplantation (HCT) using allogeneic, syngeneic, and autologous marrow. Unfortunately, the therapeutic utility of alloHCT remains limited by many severe and potentially lethal complications, including graft-versus-host disease (GVHD) and opportunistic infections (Sureda A, et al. Bone Marrow Transplant. 2015; 50(8):1037-1056).

Accordingly, there remains an unmet need for MS treatments that improve severity and outcome and that do not lead to sever GVHD and opportunistic infections. Given this need, a trial is designed for the use of a cellular therapy product of the present disclosure to treat patients with treatment-resistant PPMS.

### Study Design

This study is a prospective, open-label, single-arm phase 2 clinical trial evaluating alloHCT with a cellular therapy product of the present disclosure for the treatment of primary progressive MS. Eligible participants are primary progressive MS (PPMS) and have an eligible HLA-matched donor; a description of each participant's MS history is recorded. Eligible donors are a genotypically HLA-identical sibling or an unrelated donor who is HLA-matched by standard molecular methods. Later in the course of the study, eligibility may be opened to those participants who only have an HLA-haploidentical donor. Participants receive a MAC regimen consisting of busulfan, fludarabine and thiotepa. The graft source is granulocyte colony-stimulating factor (G-CSF)-mobilized peripheral blood mononuclear cells, which is used to produce a graft of the cellular therapy product of the present disclosure for each participant. GVHD prophylaxis consists of tacrolimus.

Participants are studied for safety and efficacy utilizing clinical assessments, laboratory testing, serial MRI of the brain and spinal cord and assessment of CSF after transplant for oligoclonal bands and neurofilaments. CSF and blood samples is collected and archived for future studies and evaluation of immune reconstitution at predefined intervals. Well-validated scoring systems is used at baseline and at intervals after transplant, specifically for activity of PPMS, quality of life, and MRI evaluations of the brain and spinal cord.

The study initially enrolls 10 participants. The active treatment period is considered up to 3 months after transplant (day 0). Evaluations of PPMS status and safety endpoints are at 1 month, 3 months, 9 months, and 12 months after transplantation. Participants are expected to be in the study for 12 months. Total study duration is expected to be 60 months (5 years) from transplant of first participant to the 2-year evaluation of the last participant who was transplanted. The primary endpoint analysis is done after all participants have completed evaluations at 1 year after transplant. A follow-up study is performed separately on all participants after transplant to assess the long-term outcome (equal to or greater than 5 years) of participants on this clinical trial.

### Donor Inclusion Criteria

1. Donors are HLA-identical sibling or HLA-matched unrelated donor. Unrelated donors are required to be matched by high resolution allele level typing for HLA-A, B, C, DRB1 and DQB1 (10/10).
2. Donors have the ability to understand and the willingness to sign a written informed consent document for stem cell harvest.
3. Donors are willing to donate PBSC for up to 2 consecutive days.
4. Donors are able to donate within North America or Hawaii at a site that employs a Spectra Optia Apheresis System for post mobilization apheresis. Donation outside of the United States may be participant to approval by relevant national or local ethics review boards.
5. Donors meet federal eligibility criteria for donors of viable, leukocyte-rich cells or tissues as defined by 21 Code of Federal Regulations (CFR) § 1271 2018 and all relevant FDA Guidance for Industry (Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; Donor Screening Recommendations to Reduce the Risk of Transmission of Zika Virus by Human Cells, Tissues, and Cellular and Tissue-Based Products, 2018).
   1. Donors determined to be ineligible based on the results of required testing and/or screening may nonetheless be included if either of the following apply, as per 21 CFR § 1271.65 2018:
   a. The donor is a first-degree or second-degree blood relative of the recipient.
   b. Urgent medical need, meaning no comparable human cell product is available and the recipient is likely to suffer death or serious morbidity without the human cell product, as attested by the investigator.
6. Donors meet any other criteria for donation as specified by standard NMDP guidelines (NMDP donors) or institutional standards (non-NMDP donors).

### Donor Exclusion Criteria

1. Identical twin
2. Pregnant or lactating females
3. Seropositivity for HIV-1 or -2 or HTLV-1 or -2
4. Current serious systemic illness including uncontrolled infections
5. Positive for anti-hepatitis C (HCV) antibody or HCV NAT
6. Malignancy within 10 years prior to donation of marrow, excluding adequately treated squamous cell skin cancer and basal cell carcinoma. Treatment has been completed (with the exception of hormonal therapy for breast cancer) with cure/remission status verified for at least 10 years at time of marrow harvest.
7. History of or symptoms consistent with multiple sclerosis or a serious autoimmune disorder.
8. History of a serious disease or disorder that could be adoptively transferred by infusion of donor hematopoietic cells.
9. Failure to meet institutional criteria for donation as described in the NMDP guidelines. Standard Practice Guideline
10. Neurological status. Donors who are HLA-identical siblings are non-concordant for multiple sclerosis on the neurological examination. MRI of the brain and spinal cord in addition to CSF examination is performed on the sibling donor within the 6 months before alloHCT and be negative for evidence of multiple sclerosis.
11. Aberrant CD45RA isoform expression by central laboratory testing
12. Women who are pregnant or breastfeeding

### Recipient Inclusion Criteria

1. Participants aged ≥18 and ≤65 years with primary progressive multiple sclerosis
2. A diagnosis of PPMS by 2017 McDonald criteria and 2013 clinical course revision (Lublin F D, et al. Neurology. 2014; 83(3):278)
3. CSF with elevated IgG index or 2 or more oligoclonal bands
4. EDSS between 2.0 and 5.5 inclusive
5. Based on review of the clinical records, there is a deterioration in the EDSS of 1 or more points over the previous 4 years.
6. Eligibility criteria confirmed by the Eligibility Review Group
7. Recipients who have been treated with ocrelizumab undergo a 90-day washout period prior to their planned day 0.
8. Recipients are willing to undergo mobilized autologous peripheral blood stem cell collection to create a cryopreserved rescue product prior to alloHCT.

### Recipient Exclusion Criteria

1. History of Progressive Multifocal Leukoencephalopathy
2. Organ dysfunction or disease that would jeopardize survival after hematopoietic cell transplantation, including but not limited to the following:
   a. Renal insufficiency as defined by an estimated GFR<60 mL/minute
   b. Cardiac dysfunction as defined by symptomatic coronary artery disease, congestive heart failure, valvular heart disease, cardiomyopathy, uncontrolled arrhythmia(s), or left ventricular ejection fraction <50%. Participants with a history of these conditions may enroll if they are demonstrated to have optimal cardiac function (as defined by echocardiography or multi-gated acquisition scan)
   c. Pulmonary dysfunction that poses a risk of mortality after transplant, defined as pre-transplant pulmonary function testing demonstrating a FEV1<70% expected and/or a DLCOadj<70% expected.
   d. Necroinflammatory or fibrotic liver disease with evidence of liver dysfunction, including but not limited to jaundice, hepatic encephalopathy, or portal hypertension
   e. Marrow dysfunction that poses a risk of peri-transplant mortality, defined as an absolute neutrophil count or lymphocyte count below the lower limit of normal, or a platelet count below 50,000/mm³.
   f. Poorly controlled hypertension despite appropriate therapy, defined as a diastolic blood pressure greater than 90 mm Hg while on therapy.
   g. Poorly controlled diabetes mellitus, defined as HgbA1c≥7.5% despite therapy or recurrent hypoglycemia while on therapy.
   h. Extreme protein-calorie malnutrition defined by Body Mass Index <18 and unintentional weight loss (3 kg in the last month or 6 kg in the last 6 months.)
3. History of smoking either tobacco or other herbal products in the last 3 months.
4. HIV seropositive.
5. HBV serology results indicating chronic HBV infection per https://www.cdc.gov/hepatitis/hbv/interpretationOfHepBSerologicResults.htm, unless HBV PCR negative. HBV seropositive participants should be on antiviral therapy after transplant.
6. HCV seropositive, unless PCR negative and having undergone ‘curative’ antiviral therapy.
7. Participant has active uncontrolled infection
8. Participant has demonstrated lack of compliance with prior medical care
9. Participants with known active malignancy
10. Participants whose life expectancy is severely limited by illness other than multiple sclerosis
11. Females who are pregnant or breast feeding.
12. Medical or psychiatric conditions that compromise ability to give informed consent or to comply with treatment protocol
13. Inability to undergo an MRI scan
14. Currently receiving treatment with investigational agents

### Donor Procedures

After scheduling, donors begin mobilization therapy with granulocyte colony stimulating factor (G-CSF, filgrastim), 10 mg/kg SC daily until completion of apheresis, as per local institutional standards, or as approved by the medical monitor.

Large volume apheresis is performed in accordance with Sponsor-recommended parameters, described in the separate Study and Administration Manual. Two days of large volume apheresis is generally necessary to achieve sufficient HSPCs for Recipient engraftment and/or sufficient Tₘₑₘ for the assigned dose cohort. Per institutional standards, plerixafor (e.g., 0.24 mg/kg SC, once) may be considered prior to the second apheresis if recommended by the Investigator to achieve the HSPC dose target.

Donors should not undergo more than 2 apheresis collections in the context of this protocol, regardless of cell yields.

### Investigational Product

The cellular therapy product of the present disclosure that is the investigational product under study in this protocol, is a cellular therapy product including up to 2 infusions. Information describing the preparation, administration, and storage of the cellular therapy product of the present disclosure is described in Example 1 above and throughout the present disclosure.

Dosing of the cellular therapy product of the present disclosure is based on Recipient actual body weight rounded to the nearest tenth of a kilogram, assessed during screening. Dosing may be adjusted to a dose based on adjusted ideal body weight (AIBW) if the participant's actual weight is greater than 120% of the ideal body weight (IBW). IBW is determined using the method of Devine.¹⁰¹ AIBW [in kg] is equal to [(actual weight−IBW)×0.40]+IBW.

All recipients have appropriate long-term central venous access placed, per institutional standard practice, prior to initiation of the conditioning regimen. Premedication with acetaminophen or paracetamol (e.g., 500-1000 mg) and diphenhydramine (e.g., 25-50 mg) should be administered prior to administration of the cellular therapy product of the present disclosure, unless contraindicated. Acetaminophen may be omitted if required by institutional policies.

The cellular therapy product of the present disclosure is administered intravenously (IV) through a central venous catheter on study day 0. If necessary to achieve target numbers of HSPC and/or Tₘₑₘ, a second dose of the cellular therapy product of the present disclosure is administered as soon as available and feasible on day 0 or day +1.

Participants meet all of the following criteria on day 0 before receiving the cellular therapy product of the present disclosure: Karnofsky performance score ≥30%, and no evidence of uncontrolled bacterial, viral or fungal infections (currently taking antimicrobial therapy and with progression or no clinical improvement).

### Concomitant Therapy

All concomitant medications, blood products, procedures, radiotherapy, or other treatments with therapeutic intent administered to recipients are recorded from the beginning of the conditioning regimen through the safety reporting period. Any concomitant therapy given for a study protocol-related adverse event (AE) should be recorded from the time of informed consent. Any treatment for AEs of special interest (AESI), disease recurrence/relapse, GVHD of any type, or PTLD are recorded all times.

Participants may not receive other investigational therapies, immunosuppressive medications, radiotherapy, or systemic antineoplastic therapy during the study except as explicitly allowed per protocol. Treatment of PTLD (section 0) should be discussed with the Medical Monitor, Sponsor and/or designee. Planned donor lymphocyte infusions (DLIs) are not permitted. Unplanned DLIs may not be administered without prior consultation with the medical monitor.

### Required Therapies

TABLE 1
Schedule of Medications From Day −6 Through Day +5

| Day | Treatment |
|---|---|
| −7 | thiotepa 5 mg/kg IV |
| −6 | thiotepa 5 mg/kg IV |
| −5 | busulfan 3.2 mg/kg IV as a single dose^{a} + fludarabine 30 mg/m² |
| | IV |
| −4 | busulfan 3.2 mg/kg IV as a single dose^{a} + fludarabine 30 mg/m² |
| | IV |
| −3 | busulfan 3.2 mg/kg IV as a single dose^{a} + fludarabine 30 mg/m² |
| | IV |
| −2 | fludarabine 30 mg/m2 IV |
| −1 | initiate tacrolimus + fludarabine 30 mg/m² IV |
| 0 | Cellular therapy product of the present disclosure infusion |
| +1 | Second dosed of cellular therapy product Infusion if not given on |
| | day 0 |

^{a}Alternatively, busulfan may be dosed to maintain an average daily AUC of 4,800 to 6,000 μM-min (19.7 to 24.6 mg*H/L) per institutional practice.

### Fludarabine

Fludarabine 30 mg/m²/day is administered over 30 to 60 minutes IV infusion on days −5 through −1. Fludarabine is dosed according to the recipient's calculated body surface area (BSA) based on the actual body weight, unless the actual body weight is greater than 120% of the ideal body weight. If the actual body weight is greater than 120% of ideal body weight, use the adjusted body weight. BSA units are (meter)², or m².

### Graft Infusion

The cellular therapy product of the present disclosure is administered on day 0 as described. A second dose of the cellular therapy product of the present disclosure (if applicable) may be administered immediately after the first dose, if available, on day 0. If the second dose arrives after the first dose, it may be administered on day +1.

### Tacrolimus

Tacrolimus is given at a dose of 1 mg IV daily from day −1 until day +180. It is recommended that the tacrolimus is initiated using the IV formulation, and then converted to a PO BID dosing schedule once a therapeutic level is achieved and the participant is tolerating oral medications. Serum levels of tacrolimus is measured around day +2 and then should be checked at least weekly (or twice weekly as clinically indicated) thereafter and the dose adjusted accordingly to maintain a level of 5 to 10 ng/mL to approximately day +180.

Tacrolimus is tapered starting at approximately day +180 with 20% dose reduction each month until discontinued. Tacrolimus may be continued if active GVHD is present. If there is nausea and vomiting at any time during tacrolimus treatment, the drug should be administered intravenously, and dose adjusted to the levels described above. Cyclosporine (target concentration 200-400 ng/mL) may be substituted for tacrolimus if the participant is intolerant of tacrolimus.

In the absence of GVHD or treatment for GVHD, tacrolimus dose is to be tapered 20% starting on approximately day +180 and then 20% dose reduction every month until discontinued. The referring physician, who receives explicit instructions and guidelines for detecting and managing GVHD, may manage this tacrolimus taper.

Blood pressure, renal function (assessed with serum creatinine, BUN), electrolytes and magnesium should be followed at least three times per week during the first month, twice weekly until day +100, then once per week until tacrolimus is tapered off (or according to institutional practice), unless clinical circumstances suggest the need for more frequent evaluations.

Monitoring and Dose Adjustments—Whole blood “trough” levels (i.e., 11-12 hours from the prior dose) of tacrolimus is evaluated weekly until day +180. Dose reductions should be made if tacrolimus toxicity (such as renal insufficiency) is present, or levels exceed 10 ng/ml in the absence of toxicity on more than 2 occasions. If toxicity or high levels are present, more frequent monitoring is initiated as clinically indicated. Dose reductions for high levels with or without toxicity should be conservative, e.g., 25%, to avoid inadequate immune suppression.

Drugs that may affect tacrolimus levels: dilantin, phenobarbital, rifampicin (may lower tacrolimus levels); and glucocorticoids, fluconazole, ketoconazole, itraconazole, voriconazole, psoaconazole cimetidine (may increase tacrolimus levels). Discontinuation of fluconazole will likely lower tacrolimus levels.

### Transfusion Support

Platelet and packed red cell transfusions are given per institutional standard of practice. Platelet counts are maintained >10,000 after transplant to prevent bleeding from the gastrointestinal tract.

### Antiovulatory Treatment

Menstruating females may be started on an anti-ovulatory agent before the initiation of the conditioning regimen, according to institutional standard of practice.

### Nutritional Support

Participants receive nutritional support according to institutional Standard Practice Guidelines including total parenteral nutrition through a central venous catheter if unable to maintain body weight by oral caloric intake.

### Permitted Concomitant Therapy

### Infection Prophylaxis

All prophylactic antibiotics may be changed or discontinued according to clinical circumstances (such as participant allergy) as determined by attending physician(s). If there are discrepancies between the recommendations below and standard of practice (reflecting current recommendations), the institutional Standard Practice Guidelines is followed.

### Antibacterial Prophylaxis

If not already on broad-spectrum antibiotics (started on day 0), once neutropenia (neutrophils <0.5×10⁹/L) occurs, prophylactic broad-spectrum oral antibiotics is started. Levofloxacin is the standard antibiotic for prophylactic use in adults over 18 years of age in many transplant centers. Other broad-spectrum antibiotics may be used based on institutional standard of practice.

### Antifungal Prophylaxis

In general, the institutional guidelines at the treatment center are followed. Recommended: Fluconazole 400 mg PO is administered daily from start of conditioning therapy until day +75 after transplant. Begin voriconazole (or other equally effective azole) if there is a pretransplant history of invasive aspergillosis or evidence of colonization with Aspergillus species or a Galactomannan (or similar diagnostic) test suggesting aspergillus infection. After transplant, if participants develop GVHD and require treatment with prednisone or methylprednisolone >1 mg/kg, stop fluconazole and start voriconazole or posaconazole. If a participant is unable to take oral agents, then start voriconazole intravenously at the therapeutic dose.

### Antiviral Prophylaxis for Prevention of HSV and VZV Infections

Antiviral prophylaxis is administered according to institutional practice. It is recommended that for participants who weigh >40 kg, valacyclovir (500 mg PO BID) or equivalent are administered from the start of conditioning therapy to day +365 after transplant for participants who are serologically positive for VZV. For participants weighing <40 kg, the dose of valacyclovir is 250 mg PO BID. Antiviral therapy is given to day +100 for participants who are serologically negative for VZV but serologically positive for HSV. Acyclovir 250 mg/m² q 12 hr IV is substituted in participants unable to tolerate oral medication. Acyclovir/valacyclovir doses is adjusted according to renal function.

### Anti-Pneumocystis Prophylaxis

For adults, trimethoprim-sulfamethoxazole DS tablet is given according to institutional standard of practice, starting after engraftment, and continuing for 1 year after transplant. Participants intolerant or allergic to trimethoprim/sulfamethoxazole are treated with dapsone, pentamidine, or atovaquone according to local institutional standard of practice. Participants are treated beyond 1 year if chronic GVHD develops requiring treatment.

### Cytomegalovirus

All cytomegalovirus (CMV)-seropositive recipients should receive letermovir prophylaxis beginning between day 0 and day +28; therapy should continue through day +100. Letermovir may be omitted for CMV-seropositive recipients only if contraindicated (e.g., known hypersensitivity to letermovir) with medical monitor approval.

Participants who are CMV seronegative and have CMV seronegative donors are administered CMV-negative screened blood products or leukodepleted blood products. The participants are monitored with a pretransplant test and then monitored weekly for CMV reactivation using CMV antigenemia or CMV DNA assays until day +60 after transplant. For participants who have CMV diagnosed before day +60, participants continue to be monitored until 1 year after transplant.

Participants who are CMV seropositive or who have seropositive donors are monitored weekly for CMV reactivation using CMV antigenemia or CMV DNA assays after HCT beginning on about day 0 and continuing until discharge or day +100. CMV monitoring occurs at least every other week during follow-up visits until 6 months post-HCT. For those participants that have reactivated CMV before day 100, or for those who are taking prednisone for chronic GVHD after day 100, weekly CMV monitoring continues to 1 year after transplant according to institutional practice. Letermovir may be administered according to institutional guidelines. CMV prophylaxis, treatment and monitoring is according to institutional guidelines.

Participants treated for CMV reactivation should have weekly CMV testing for 3 weeks prior to reinitiating every other week monitoring.

### Epstein Barr Virus (EBV)

Baseline studies (pretransplant):
Serology for antibodies, including antibody titers, to EBV viral capsid antigen and/or nuclear antigen.
EBV PCR amplification to evaluate viral load in the peripheral blood.

Posttransplant surveillance monitoring beginning at day +14 after transplant, through day +100 after transplant:
EBV PCR weekly or according to institutional standard of practice
EBV PCR, perform twice per week, if there is evidence of rising copy number

Posttransplant surveillance monitoring from day +101 to 6 months after transplant:
EBV PCR, perform every other week
If the test is positive, then perform EBV PCR twice per week.

Participants who develop a viral load of >1000 copies per mL plasma receive further individualized monitoring and/or therapy according to institutional guidelines.

### Additional Viral Monitoring

Additional viral monitoring may be conducted according to institutional guidelines. For example, monitoring for HHV6 reactivation or adenovirus infections may be performed based on clinical indications. Monitoring for coronavirus infections (including SARS-CoV2 and others) before or after transplant is conducted according to institutional guidelines.

### Treatment of GVHD

First-line therapy is 1 or 2 mg/kg of prednisone or methylprednisolone. For participants who do not respond to treatment, second-line therapy includes additional immunosuppressive therapy as clinically indicated. Ruxolitinib has been FDA-approved for treatment of steroid-refractory aGVHD.

Management of cGVHD is based on the recommendations of physicians experienced in the care of transplant survivors with cGVHD. Ibrutinib, belumosudil and ruxolitinib have been FDA-approved for treatment of treatment-refractory cGVHD.

### Management of Graft Rejection

It is expected that participants will not have recovery of autologous hematopoiesis after graft rejection. Participants are monitored and treated appropriately to prevent opportunistic infections during this recovery period. If participants experience protracted neutropenia caused by graft rejection, this is reviewed by the study team, and a decision is made about a rescue transplant using the recipient's cryopreserved mobilized autologous PBSCs or a second alloHCT.

### Withdrawal From Trial

Participants are considered to have completed the study if they have undergone transplantation and have completed 2 years of follow-up.

### AEs and SAEs

### Definitions of AE and SAE

AE—Any untoward medical occurrence in a participant or clinical investigation participant administered a pharmaceutical product, medical treatment or procedure and which does not necessarily have to have a causal relationship with this treatment. An AE can therefore be any unfavorable and unintended sign (including an abnormal laboratory finding, for example), symptom, or disease temporally associated with the use of a medicinal product, medical treatment or procedure whether or not considered related to the medicinal/investigational product.
Life-threatening AE—Any AE that places the participant or participant, in the view of the investigator, at immediate risk of death from the reaction.
Unexpected AE—An AE is “unexpected” when its nature (specificity), severity, or frequency are not consistent with (a) the known or foreseeable risk of AEs associated with the research procedures described in the protocol-related documents, such as the IRB-approved research protocol, informed consent document and other relevant sources of information such as product labeling and package inserts; and are also not consistent with (b) the characteristics of the participant population being studied including the expected natural progression of any underlying disease, disorder or condition any predisposing risk fact or profile for the AE.

A listing of expected AEs for the participant population are known.
SAE—An untoward medical occurrence that results in any of the following outcomes:
   Death
   Life-threatening AE (real risk of dying)
   Inpatient hospitalization or prolongation of existing hospitalization. Hospitalizations commonly associated with transplants including, but not limited, to febrile neutropenia, fevers, infections, and gastrointestinal toxicities are not recorded as SAEs as the majority of participants receiving transplants are hospitalized.
   A persistent or significant disability/incapacity or substantial disruption of the ability to conduct normal life functions
   A congenital anomaly/birth defect
   Requires intervention to prevent permanent impairment of damage
Attribution—The following are definitions for determining whether an AE is related to a medical product, treatment or procedure:
An AE is “related or possibly related to the research procedures” if, in the opinion of the principal investigator, it was more likely than not caused by the research procedures.
AEs that are solely caused by an underlying disease, disorder or condition of the participant or by other circumstances unrelated to either the research or any underlying disease, disorder or condition of the participant are not “related or possibly related.”
If there is any question whether or not an AE is related or possibly related, the AE should be reported.

### Identifying AEs and SAEs

The investigator should attempt to establish a diagnosis of the event on the basis of signs, symptoms and/or other clinical information. In such cases, the diagnosis should be documented as an AE and/or SAE and not described as the individual signs or symptoms. The following information should be recorded:
Description of the AE using concise medical terminology
Description as to whether or not the AE is serious
The start date (date of AE onset)
The maximum severity (grade) of the AE
A description of the potential relatedness of the AE to study drug or a study procedure

Evaluation for cGVHD is performed at month 3 (day +80 to +100) and month 12 (day +365±20 days) per the SoA. cGVHD should be graded per the 2015 NIH Consensus guidelines (Jagasia M H, et al. Biol Blood Marrow Transplant J Am Soc Blood Marrow Transplant. 2015; 21(3):389-401).

### AE Severity

All AEs are graded in severity according to the modified (for HSCT) NCI Common Toxicity Criteria for Adverse Events (CTCAE) Version 5.0 dated Nov. 27, 2017. The scale in its entirety can be found at: ctep.cancer.gov/protocolDevelopment.

When CTCAE Version 5.0 criteria cannot be used, the AE should be graded as defined below:
Grade 1: The AE is transient and easily tolerated by the participant (mild).
Grade 2: The AE causes the participant discomfort and interrupts the participant's usual activities (moderate).
Grade 3 or 4: The AE causes considerable interference with the participant's usual activities and may be incapacitating or life threatening (severe).
Grade 5: The AE resulted in death of the participant (severe).

An event is defined as serious when it meets at least 1 of the predefined outcomes as described in the definition of an SAE, NOT when it is rated as severe.

### Assessment of Causality

Association or relatedness to the study agent is assessed by the investigator as follows:
Definite: The event follows a reasonable temporal sequence from exposure to the investigational agent, has been previously described in association with the investigational agent and cannot reasonably be attributed to other factors such as the participant's clinical state, other therapeutic interventions or concomitant medications; AND the event disappears or improves with withdrawal of the investigational agent and/or re-appears on re-exposure (e.g., in the event of an infusion reaction).
Probable: The event follows a reasonable temporal sequence from exposure to the investigational agent and has been previously described in association with the investigational agent OR cannot reasonably be attributed to other factors such as the participant's clinical state, other therapeutic interventions or concomitant medications.
Possible: The event follows a reasonable temporal sequence from exposure to the investigational agent but could be attributable to other factors such as the participant's clinical state, other therapeutic interventions, or concomitant medications.
Unlikely: Toxicity is doubtfully related to the investigational agent(s). The event may be attributable to other factors such as the participant's clinical state, other therapeutic interventions or concomitant medications.
Unrelated: The event is clearly related to other factors such as the participant's clinical state, other therapeutic interventions, or concomitant medications.

For general AE assessment, an AE is considered related if it is assessed as definitely, probably, or possibly related; unrelated if it is assessed as unlikely related or unrelated.

### AEs of Special Interest

AESIs for this study include:
Any grade ≥2 aGVHD
Moderate-to-severe cGVHD (graded per 2015 NIH consensus criteria; Jagasia M H, et al. Biol Blood Marrow Transplant J Am Soc Blood Marrow Transplant. 2015; 21(3):389-401)
PTLD
Grade ≥2 infections
Primary and secondary graft failure

### GVHD

GVHD is a known complication of HCT and may be primarily diagnosed and managed according to local institutional standards. However, the following protocol-related guidelines apply.

### Staging and Grading of GVHD

aGVHD is graded per MAGIC criteria (Harris A C, et al. Biol Blood Marrow Transplant J Am Soc Blood Marrow Transplant. 2016; 22(1):4-10).

cGVHD is graded per 2015 NIH consensus criteria (Jagasia M H, et al. Biol Blood Marrow Transplant J Am Soc Blood Marrow Transplant. 2015; 21(3):389-401).

### PTLD

PTLD is defined as a lymphoma that occurs after HCT or solid organ transplantation. It is a known complication of HCT and may be generally diagnosed and treated according to local institutional standards. However, the following protocol-related guidelines apply.

Participants suspected of PTLD (e.g., fever of unknown origin >39° C. with lymphadenopathy and/or hepatosplenomegaly) should undergo appropriate imaging studies (e.g., computed tomography [CT] and/or positron emission tomography [PET] scanning of at least the chest and abdomen) and testing for EBV viremia. Biopsies should be sought and include studies of routine morphology, immunophenotyping, and EBV (e.g., EBER in situ hybridization) testing.

The diagnosis of PTLD requires a biopsy consistent with the 2017 World Health Organization (WHO) classification of PTLD (nondestructive [plasmacytic hyperplasia, infectious mononucleosis—like, and florid follicular hyperplasia], polymorphic, monomorphic or Hodgkin lymphoma-like), along with lymphoma type-appropriate staging procedures such as computed tomography (CT) with or without F-fluorodeoxyglucose positron emission tomography (FDG PET) (Swerdlow S H. WHO Classification of Tumours of Haematopoietic and Lymphoid Tissues. Vol 2. Revised 4th Edition. IARC Press; 2017:453-462; Swerdlow S H, et al. Blood. 2016; 127(20):2375-2390; Dierickx D, et al. N Engl J Med. 2018; 378(6):549-562).

### Objectives and Endpoints

### Primary Objective and Associated Endpoint

| Primary Objective | Primary Endpoint |
|---|---|
| To evaluate the safety of alloHCT with | Severe aGVHD-free survival (SAGFS) at 180 days after |
| cellular therapy product of the present | infusion of the cellular therapy product of the present |
| disclosure as treatment for PPMS | disclosure, defined as alive without a history of grade ≥3 |
| | aGVHD (graded per MAGIC criteria (Harris A C, et al. |
| | Biol Blood Marrow Transplant J Am Soc Blood Marrow |
| | Transplant. 2016; 22(1): 4-10) |

Abbreviations: alloHCT, allogeneic hematopoietic cell transplantation; aGVHD, acute graft-versus-host disease; MAGIC, Mount Sinai Acute GVHD International Consortium; PPMS, primary progressive multiple sclerosis.

### Secondary Objectives and Associated Endpoints

| Secondary Objectives | Secondary Endpoints |
|---|---|
| To evaluate the treatment effect on | EFS at 9 and 12 months after infusion of the cellular therapy |
| PPMS activity and severity | product of the present disclosure, defined as alive and free of |
| | PPMS (neurologically stable). An event is defined as sustained |
| | loss of neurological function as measured by EDSS (>1 point), |
| | relapse, 2 or more new or larger (active) lesions on the MRI, or |
| | death. |
| | Assessment of loss of neurological function as measured by |
| | EDSS (>1 point) or relapse every 3 months after infusion of the |
| | cellular therapy product of the present disclosure through month |
| | 18, then at 24 months |
| | OS at 1 and 2 years from the time of treatment assignment. Death |
| | due to any cause is the event for this endpoint. Participants alive |
| | at the time of last contact are censored. |
| | -TRM, defined as death occurring at any time after start of |
| | alloHCT that is definitely or probably resulting from study |
| | treatment and not associated with other causes. Cause of death is |
| | determined after reviewing clinical and autopsy data. |
| | Disease activity- MRI scans of the brain and spinal cord are done |
| | at baseline, at 1 and 3 months after infusion of the cellular therapy |
| | product of the present disclosure, and then annually at 1 and 2 |
| | years after infusion of the cellular therapy product of the present |
| | disclosure. Additional MRIs are done as clinically indicated. |
| | Incidence of disease-modifying drugs for PPMS initiated after |
| | infusion of the cellular therapy product of the present disclosure. |
| | Participants are not expected to receive additional disease- |
| | modifying therapy for PPMS in the absence of disease activity, as |
| | defined above. A decision to initiate disease-modifying therapy |
| | other than that specified in the protocol after alloHCT is |
| | considered treatment failure if participants have otherwise met the |
| | definition of treatment failure based on loss of neurological |
| | dysfunction or disease activity. In general, this would include the |
| | administration of any therapy (drugs, biologics, or any other |
| | treatments) clearly given as immunomodulatory therapy for |
| | underlying PPMS. If these agents are used and there is no disease |
| | activity or loss of neurological function, then the participants are |
| | censored for further follow-up. Use of immunosuppressive agents |
| | for the treatment of GVHD or other transplant-related condition |
| | are not considered for this endpoint. |
| To evaluate the safety of alloHCT | Regimen-related toxicity - AEs are defined by the modified |
| as determined by regimen -related | CTCAE version 5.0. AEs are tabulated for each participant. |
| toxicities, infectious | Incidence and severity of GVHD. The grading of aGVHD and |
| complications, acute and chronic | cGVHD follows previously published guidelines but also includes |
| GVHD, treatment-related | capture of symptoms and characterization of alternative causes. |
| mortality, overall mortality, graft | The highest level of organ abnormalities, the etiologies |
| failure, and time to engraftment | contributing to the abnormalities, and biopsy results pertaining to |
| | GVHD are identified. All diagnostic biopsies of these organs are |
| | reviewed by pathologists experienced in the diagnosis of GVHD. |
| | Incidence of primary and secondary graft failure. The definitions |
| | of primary and secondary graft failure are based on the EBMT |
| | Handbook's definitions of these events(Carreras E, et al. The |
| | EBMT Handbook: Hematopoietic Stem Cell Transplantation and |
| | Cellular Therapies. Published online 2019). Recovery of host |
| | hematopoiesis after rejection is also assessed. |
| | Development of infectious complications. The incidence of |
| | definite and probable viral, fungal, and bacterial infections are |
| | tabulated for each participant. |

Abbreviations: AE, adverse event; aGVHD, acute GVHD; alloHCT, allogeneic hematopoietic cell transplantation; cGVHD, chronic GVHD; CTCAE, Common Terminology Criteria for Adverse Events; EBMT, European Society for Blood and Marrow Transplantation; EDSS, Expanded Disability Status Scale; EFS, event-free survival; GVHD, graft-versus-host disease; MRI, magnetic resonance imaging; OS, overall survival; PPMS, primary progressive multiple sclerosis; SAE, serious adverse event; TRM, treatment related- mortality.

### Exploratory Objectives and Associated Endpoints

| Exploratory Objective(s) | Exploratory Endpoints |
|---|---|
| To evaluate the effect of alloHCT | Clearance of CSF oligoclonal bands at 2 years after alloHCT |
| on biomarkers of MS including | |
| oligoclonal bands and | |
| neurofilaments in the CSF | |
| To evaluate the effect of alloHCT | QOL as measured using previously validated questionnaires for |
| on QOL in participants with | adults: the MSQOL-54, SF36, MSIS-29, and PDDS |
| PPMS | |
| To evaluate the effect of | The cumulative number of new or enlarging T2 lesions on brain MRI |
| alloHCT on well-established | from randomization (day 0) to the follow-up evaluations |
| defined radiographic changes | The cumulative number of new gadolinium-enhancing lesions on |
| associated with PPMS. | brain MRI from randomization (day 0) to the follow-up evaluations |
| | T2 lesion volume change from randomization (day 0) to the follow-up |
| | evaluations |
| | T1 lesion volume change from randomization (day 0) to the follow-up |
| | evaluations |
| | Cortical gray matter volume change from randomization (day 0) to the |
| | follow-up evaluations |
| | Thalamic volume change from randomization (day 0) to the follow-up |
| | evaluations |
| | Whole brain volume change from randomization (day 0) to the follow- |
| | up evaluations |

Abbreviations: alloHCT, allogeneic hematopoietic cell transplantation; CSF, cerebrospinal fluid; MRI, magnetic resonance imaging; MS, multiple sclerosis; PDDS, Patient Determined Disease Steps; PPMS, primary progressive multiple sclerosis; QOL, quality of life.

### Interpretations

All references, patents and patent applications disclosed herein are incorporated by reference with respect to the subject matter for which each is cited, which in some cases may encompass the entirety of the document. The entire contents of WO2018170335; U.S. Pat. Nos. 10,300,090; and 10,857,183 are incorporated herein by reference.

The terminology used herein is for the purpose of describing particular cases only and is not intended to be limiting.

The indefinite articles “a” “an,” and “the” as used herein in the specification and in the claims, unless clearly indicated to the contrary, should be understood to mean “at least one.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

By “one or more” or “at least one” is meant at least one, e.g., one, two, three, four, five, six, seven, eight, nine, ten or more.

The term “similar” is understood to be resembling up to and including identical. Therefore two (or more) items may be identical, substantially identical, comprise equivalent components, comprise substitutable components, comprise analogous components, comprise comparable components, comprise complementary components, comprise related components, comprise like components, and/or the differences between the two (or more items) are insubstantial and/or result in a composition having equivalent properties, identical properties, substantially identical properties, and the like.

The terms “patient” and “subject” are synonyms. As used herein, the term “patient weight” may be either the patient's “actual weight” or the patient's “ideal weight”.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “with,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any aspect or embodiment described herein can be combined with any other aspect or embodiment as disclosed herein.

1. A cellular therapy product comprising a population of isolated CD45⁺ cells, wherein the population of isolated CD45⁺ cells comprises:
a) a dose of approximately 1.0×10⁵ to approximately 1.0×10⁸ CD34⁺ hematopoietic stem and progenitor cells (HSPC) per kilogram of body weight of a human subject receiving the product or a total dose of approximately 5.0×10⁵ to approximately 1.5×10¹⁰ CD34⁺ hematopoietic stem and progenitor cells (HSPC);
b) a dose of approximately 1.0×10⁵ to approximately 1.0×10⁸ CD45RA⁻ memory T cells (Tₘₑₘ) per kilogram of body weight of the human subject receiving the product or a total dose of approximately 5.0×10⁵ to approximately 1.5×10¹⁰ CD45RA⁻ memory T cells (Tₘₑₘ); and
c) a dose of approximately 1.0×10⁵ to approximately 2.0×10⁷ fresh CD4⁺CD25⁺CD127^{dim} regulatory T cells (Treg) per kilogram of body weight of the human subject receiving the product or a total dose of approximately 5.0×10⁵ to approximately 3.0×10⁹ isolated fresh ⁺CD4⁺CD25⁺CD127^{dim} regulatory T cells (Treg),
wherein the cellular therapy product has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), and
wherein the population of isolated CD45⁺ cells is formulated with an excipient at a neutral pH.

2. The cellular therapy product of claim 1, wherein:
a) the dose of HSPC comprises a dose of approximately 1.0×10⁵ or more HSPC per kilogram of body weight of the human subject receiving the product, approximately 2.0×10⁵ or more HSPC per kilogram of body weight of the human subject receiving the product, approximately 3.0×10⁵ or more HSPC per kilogram of body weight of the human subject receiving the product, approximately 4.0×10⁵ or more HSPC per kilogram of body weight of the human subject receiving the product, approximately 5.0×10⁵ or more HSPC per kilogram of body weight of the human subject receiving the product, approximately 6.0×10⁵ or more HSPC per kilogram of body weight of the human subject receiving the product, approximately 7.0×10⁵ or more HSPC per kilogram of body weight of the human subject receiving the product, approximately 8.0×10⁵ or more HSPC per kilogram of body weight of the human subject receiving the product, approximately 9.0×10⁵ or more HSPC per kilogram of body weight of the human subject receiving the product, approximately 1.0×10⁶ or more HSPC per kilogram of body weight of the human subject receiving the product, approximately 1.5×10⁶ or more HSPC per kilogram of body weight of the human subject receiving the product, approximately 2.0×10⁶ or more HSPC per kilogram of body weight of the human subject receiving the product, approximately 2.5×10⁶ or more HSPC per kilogram of body weight of the human subject receiving the product, approximately 3.0×10⁶ or more HSPC per kilogram of body weight of the human subject receiving the product, approximately 3.5×10⁶ or more HSPC per kilogram of body weight of the human subject receiving the product, approximately 4.0×10⁶ or more HSPC per kilogram of body weight of the human subject receiving the product, approximately 4.5×10⁶ or more HSPC per kilogram of body weight of the human subject receiving the product, approximately 5.0×10⁶ or more HSPC per kilogram of body weight of the human subject receiving the product, approximately 5.5×10⁶ or more HSPC per kilogram of body weight of the human subject receiving the product, approximately 6.0×10⁶ or more HSPC per kilogram of body weight of the human subject receiving the product, approximately 6.5×10⁶ or more HSPC per kilogram of body weight of the human subject receiving the product, approximately 7.0×10⁶ or more HSPC per kilogram of body weight of the human subject receiving the product, approximately 7.5×10⁶ or more HSPC per kilogram of body weight of the human subject receiving the product, approximately 8.0×10⁶ or more HSPC per kilogram of body weight of the human subject receiving the product, approximately 8.5×10⁶ or more HSPC per kilogram of body weight of the human subject receiving the product, approximately 9.0×10⁶ or more HSPC per kilogram of body weight of the human subject receiving the product, approximately 9.5×10⁶ or more HSPC per kilogram of body weight of the human subject receiving the product, approximately 1.0×10⁷ or more HSPC per kilogram of body weight of the human subject receiving the product, approximately 1.5×10⁷ or more HSPC per kilogram of body weight of the human subject receiving the product, approximately 2.0×10⁷ or more HSPC per kilogram of body weight of the human subject receiving the product, approximately 2.5×10⁷ or more HSPC per kilogram of body weight of the human subject receiving the product, approximately 3.0×10⁷ or more HSPC per kilogram of body weight of the human subject receiving the product, approximately 3.5×10⁷ or more HSPC per kilogram of body weight of the human subject receiving the product, approximately 4.0×10⁷ or more HSPC per kilogram of body weight of the human subject receiving the product, approximately 4.5×10⁷ or more HSPC per kilogram of body weight of the human subject receiving the product, approximately 5.0×10⁷ or more HSPC per kilogram of body weight of the human subject receiving the product, approximately 5.5×10⁷ or more HSPC per kilogram of body weight of the human subject receiving the product, approximately 6.0×10⁷ or more HSPC per kilogram of body weight of the human subject receiving the product, approximately 6.5×10⁷ or more HSPC per kilogram of body weight of the human subject receiving the product, approximately 7.0×10⁷ or more HSPC per kilogram of body weight of the human subject receiving the product, approximately 7.5×10⁷ or more HSPC per kilogram of body weight of the human subject receiving the product, approximately 8.0×10⁷ or more HSPC per kilogram of body weight of the human subject receiving the product, approximately 8.5×10⁷ or more HSPC per kilogram of body weight of the human subject receiving the product, approximately 9.0×10⁷ or more HSPC per kilogram of body weight of the human subject receiving the product, approximately 9.5×10⁷ or more HSPC per kilogram of body weight of the human subject receiving the product, or approximately 1.0×10⁸ or more HSPC per kilogram of body weight of the human subject receiving the product; and/or
b) the dose of HSPC comprises a total dose of approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC; and/or
c) the dose of Tmem comprises a dose of approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product; and/or
d) the dose of Tmem comprises a total dose of approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem; and/or
e) the dose of fresh Treg comprises a dose of approximately 1.0×10⁵ or more fresh Treg per kilogram of body weight of the human subject receiving the product, approximately 2.0×10⁵ or more fresh Treg per kilogram of body weight of the human subject receiving the product, approximately 3.0×10⁵ or more fresh Treg per kilogram of body weight of the human subject receiving the product, approximately 4.0×10⁵ or more fresh Treg per kilogram of body weight of the human subject receiving the product, approximately 5.0×10⁵ or more fresh Treg per kilogram of body weight of the human subject receiving the product, approximately 6.0×10⁵ or more fresh Treg per kilogram of body weight of the human subject receiving the product, approximately 7.0×10⁵ or more fresh Treg per kilogram of body weight of the human subject receiving the product, approximately 8.0×10⁵ or more fresh Treg per kilogram of body weight of the human subject receiving the product, approximately 9.0×10⁵ or more fresh Treg per kilogram of body weight of the human subject receiving the product, approximately 1.0×10⁶ or more fresh Treg per kilogram of body weight of the human subject receiving the product, approximately 1.5×10⁶ or more fresh Treg per kilogram of body weight of the human subject receiving the product, approximately 2.0×10⁶ or more fresh Treg per kilogram of body weight of the human subject receiving the product, approximately 2.5×10⁶ or more fresh Treg per kilogram of body weight of the human subject receiving the product, approximately 3.0×10⁶ or more fresh Treg per kilogram of body weight of the human subject receiving the product, approximately 3.5×10⁶ or more fresh Treg per kilogram of body weight of the human subject receiving the product, approximately 4.0×10⁶ or more fresh Treg per kilogram of body weight of the human subject receiving the product, approximately 4.5×10⁶ or more fresh Treg per kilogram of body weight of the human subject receiving the product, approximately 5.0×10⁶ or more fresh Treg per kilogram of body weight of the human subject receiving the product, approximately 5.5×10⁶ or more fresh Treg per kilogram of body weight of the human subject receiving the product, approximately 6.0×10⁶ or more fresh Treg per kilogram of body weight of the human subject receiving the product, approximately 6.5×10⁶ or more fresh Treg per kilogram of body weight of the human subject receiving the product, approximately 7.0×10⁶ or more fresh Treg per kilogram of body weight of the human subject receiving the product, approximately 7.5×10⁶ or more fresh Treg per kilogram of body weight of the human subject receiving the product, approximately 8.0×10⁶ or more fresh Treg per kilogram of body weight of the human subject receiving the product, approximately 8.5×10⁶ or more fresh Treg per kilogram of body weight of the human subject receiving the product, approximately 9.0×10⁶ or more fresh Treg per kilogram of body weight of the human subject receiving the product, approximately 9.5×10⁶ or more fresh Treg per kilogram of body weight of the human subject receiving the product, approximately 1.0×10⁷ or more fresh Treg per kilogram of body weight of the human subject receiving the product, approximately 1.5×10⁷ or more fresh Treg per kilogram of body weight of the human subject receiving the product, or approximately 2.0×10⁷ or more fresh Treg per kilogram of body weight of the human subject receiving the product; and/or
f) the dose of fresh Treg comprises a total dose of approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg; and/or
g) the cellular therapy product comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product; and/or
h) the cellular therapy product comprises less than approximately 6.0×10⁷ total Tcon, less than approximately 5.5×10⁷ total Tcon, less than approximately 5.0×10⁷ total Tcon, less than approximately 4.5×10⁷ total Tcon, less than approximately 4.0×10⁷ total Tcon, less than approximately 3.5×10⁷ total Tcon, less than approximately 3.0×10⁷ total Tcon, less than approximately 2.5×10⁷ total Tcon, less than approximately 2.0×10⁷ total Tcon, less than approximately 1.5×10⁷ total Tcon, less than approximately 1.0×10⁷ total Tcon, less than approximately 9.5×10⁶ total Tcon, less than approximately 9.0×10⁶ total Tcon, less than approximately 8.5×10⁶ total Tcon, less than approximately 8.0×10⁶ total Tcon, less than approximately 7.5×10⁶ total Tcon, less than approximately 7.0×10⁶ total Tcon, less than approximately 6.5×10⁶ total Tcon, less than approximately 6.0×10⁶ total Tcon, less than approximately 5.5×10⁶ total Tcon, less than approximately 5.0×10⁶ total Tcon, less than approximately 4.5×10⁶ total Tcon, less than approximately 4.0×10⁶ total Tcon, less than approximately 3.5×10⁶ total Tcon, less than approximately 3.0×10⁶ total Tcon, less than approximately 2.5×10⁶ total Tcon, less than approximately 2.0×10⁶ total Tcon, less than approximately 1.5×10⁶ total Tcon, or less than approximately 1.0×10⁶ total Tcon.

3. The cellular therapy product of claim 1, wherein:
a) the population of isolated CD45⁺ cells is formulated at a volume that ranges from approximately 5 mL to approximately 1 L; and/or
b) the population of isolated CD45⁺ cells is formulated at a volume of approximately 5 mL, approximately 10 mL, approximately 15 mL, approximately 20 mL, approximately 25 mL, approximately 30 mL, approximately 35 mL, approximately 40 mL, approximately 45 mL, approximately 50 mL, approximately 55 mL, approximately 60 mL, approximately 65 mL, approximately 70 mL, approximately 75 mL, approximately 80 mL, approximately 85 mL, approximately 90 mL, approximately 95 mL, approximately 100 mL, approximately 125 mL, approximately 150 mL, approximately 175 mL, approximately 200 mL, approximately 225 mL, approximately 250 mL, approximately 275 mL, approximately 300 mL, approximately 325 mL, approximately 350 mL, approximately 375 mL, approximately 400 mL, approximately 425 mL, approximately 450 mL, approximately 475 mL, approximately 500 mL, approximately 525 mL, approximately 550 mL, approximately 575 mL, approximately 600 mL, approximately 625 mL, approximately 650 mL, approximately 675 mL, approximately 700 mL, approximately 725 mL, approximately 750 mL, approximately 775 mL, approximately 800 mL, approximately 825 mL, approximately 850 mL, approximately 875 mL, approximately 900 mL, approximately 925 mL, approximately 950 mL, approximately 975 mL, or approximately 1 L; and/or
c) the neutral pH ranges from approximately 6.8 to approximately 7.6; and/or
d) the excipient comprises a transport buffer, optionally wherein the transport buffer comprises approximately 120 to approximately 160 mEq sodium and/or the transport buffer comprises approximately 270 to approximately 320 mOsmol/L total, optionally wherein the transport buffer is selected from the group consisting of: phosphate-buffered saline (PBS), human serum, PlasmaLyte, Normosol-R, and any combination thereof, optionally wherein the transport buffer further comprises approximately 0.1% volume by volume to approximately 10% volume by volume of a human carrier protein, optionally wherein the human carrier protein is selected from the group consisting of: human serum albumin (HSA), intravenous immune globulin (IVIG), AB serum, and any combination thereof; and/or
e) the population of isolated CD45⁺ cells is formulated in a single dose transfer bag, optionally wherein the single dose transfer bag is a polyvinyl chloride (PVC) transfer bag or an ethylene vinyl acetate (EVA) transfer bag; and/or
f) the population of isolated CD45⁺ cells is enriched for HSPC, Tmem, and Treg; and/or
g) the population of isolated CD45⁺ cells comprises a ratio of Tcon to HSPC that is less than 1:3, less than 1:5, less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, less than 1:50,000, less than 1:55,000, less than 1:60,000, less than 1:65,000, less than 1:70,000, less than 1:75,000, less than 1:80,000, less than 1:85,000, less than 1:90,000, less than 1:95,000, less than 1:100,000, less than 1:200,000, less than 1:300,000, less than 1:400,000, less than 1:500,000, less than 1:600,000, less than 1:700,000, less than 1:800,000, less than 1:900,000, or less than 1:1,000,000; and/or
h) the population of isolated CD45⁺ cells comprises a ratio of Tcon to Tmem this is less than 1:10, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000; and/or
i) the population of isolated CD45⁺ cells comprises a ratio of Tcon to Treg that is less than 1:1, less than 1:2, less than 1:3, less than 1:4, less than 1:5, less than 1:6, less than 1:7, less than 1:8, less than 1:9, less than 1:10, less than 1:11, less than 1:12, less than 1:13, less than 1:14, less than 1:15, less than 1:16, less than 1:17, less than 1:18, less than 1:19, less than 1:20, less than 1:30, less than 1:40, less than 1:50, less than 1:60, less than 1:70, less than 1:80, less than 1:90, less than 1:100, less than 1:200, less than 1:300, less than 1:400, less than 1:500, less than 1:600, less than 1:700, less than 1:800, less than 1:900, less than 1:1,000, less than 1:1,500, less than 1:2,000, less than 1:2,500, less than 1:3,000, less than 1:3,500, less than 1:4,000, less than 1:4,500, less than 1:5,000, less than 1:5,500, less than 1:6,000, less than 1:6,500, less than 1:7,000, less than 1:7,500, less than 1:8,000, less than 1:8,500, less than 1:9,000, less than 1:9,500, less than 1:10,000, less than 1:15,000, less than 1:20,000, less than 1:25,000, less than 1:30,000, less than 1:35,000, less than 1:40,000, less than 1:45,000, or less than 1:50,000; and/or
j) the population of isolated CD45⁺ cells comprises a ratio of HSPC to Tₘₑₘ that is approximately 500:1 to approximately 1:1,000; and/or
k) the population of isolated CD45⁺ cells comprises a ratio of HSPC to Treg that is from approximately 100:1 to approximately 1:30; and/or
1) the population of isolated CD45⁺ cells comprises a ratio of Tₘₑₘ to Treg that is from approximately 2000:1 to approximately 1:10; and/or
m) the population of isolated CD45⁺ cells further comprises an enriched population of TCR Vα24Jα18⁺CD127⁺ invariant Natural Killer T cells (iNKT), optionally wherein the population of isolated CD45⁺ cells comprises a dose of approximately 5.0×10² to approximately 2.0×10⁶ iNKT per kilogram of body weight of the human subject receiving the product or a total dose of approximately 2.0×10³ to approximately 4.0×10⁸ iNKT, optionally wherein the dose of iNKT comprises a dose of approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product, optionally wherein the dose of iNKT comprises a total dose of approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT, optionally wherein the population of isolated CD45⁺ cells comprises a ratio of HSPC to iNKT that is from approximately 1:2 to approximately 500,000:1, optionally wherein the population of isolated CD45⁺ cells comprises a ratio of iNKT to Tcon is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100, optionally wherein the population of isolated CD45⁺ cells comprises a ratio of iNKT to Tmem that is from approximately 5:1 to approximately 1:1,000,000, optionally wherein the iNKT are CD1d-tet⁺, 6B11⁺, or CD1d-tet⁺6B11⁺; and/or
n) the HSPC are cKIT⁺, CD133⁺, CD90⁺, CD38⁻, CD45RA⁻, Lin⁻, CD19⁻, TCRα⁻, or any combination thereof; and/or
o) the Tmem are CD3⁺CD45RO⁺ and/or
p) the Tmem comprise a population of T central memory cells (T_{CM}), a population of T effector memory cells (T_{EM}), a population of T stem central memory cells (T_{SCM}), or any combination thereof, optionally wherein the T_{CM} are CD4⁺CD45RO⁺ or CD8⁺CD45RO⁺ and/or the T_{CM} are CD45RA−, CD62L⁺, CCR7⁺, or any combination thereof, optionally wherein the T_{EM} are CD4+, CD45RO⁺, CD45RA−, CD62L⁻, CCR7, or any combination thereof, optionally wherein the T_{SCM} are CD4⁺CD45RA+ or CD8⁺CD45RA⁺ and/or the T_{SCM} are CD95⁺, CD122⁺, CXCR3⁺, LFA-1⁺, or any combination thereof; and/or
q) the Treg are FoxP3+; and/or
r) the Treg comprise a population of naïve Treg cells, a population of memory Treg cells, or a population of naïve Treg cells and memory Treg cells, optionally wherein the naïve Treg cells are CD4⁺CD25⁺CD127^{dim}FoxP3⁺CD45RA⁺CD45RO⁻, optionally wherein the dose of Treg comprises a dose of approximately 2.0×10⁵ to approximately 5.0×10⁸ naïve Treg cells per kilogram of body weight of the human subject receiving the product, or a total dose of approximately 8.0×10⁵ to approximately 1.0×10¹¹ naïve Treg cells; optionally wherein the population of isolated CD45⁺ cells comprises a ratio of HSPC to naïve Treg that is from approximately 1:500 to approximately 100:1, optionally wherein the population of isolated CD45⁺ cells comprises a ratio of Tmem to naïve Treg that is from approximately 3:1 to approximately 1:10, optionally wherein the population of isolated CD45⁺ cells comprises a ratio of Tcon to naïve Treg that is less than 1:1, less than 1:2, less than 1:3, less than 1:4, less than 1:5, less than 1:6, less than 1:7, less than 1:8, less than 1:9, or less than 1:10, optionally wherein the memory Treg cells are CD4⁺CD25⁺CD127^{dim}FoxP3⁺CD45RA-CD45RO⁺, optionally wherein the dose of Treg comprises a dose of approximately 5.0×10⁴ to approximately 5.0×10⁸ memory Treg cells per kilogram of body weight of the human subject receiving the product, or a total dose of approximately 2.0×10⁵ to approximately 1.0×10¹¹ memory Treg cells, optionally wherein the population of isolated CD45⁺ cells comprises a ratio of HSPC to memory Treg that is from approximately 1:500 to approximately 10,000:1, optionally wherein the population of isolated CD45⁺ cells comprises a ratio of Tₘₑₘ to memory Treg that is from approximately 27:1 to approximately 9:10, optionally wherein the population of isolated CD45⁺ cells comprises a ratio of Tcon to memory Treg is less than 1:1, less than 1:2, less than 1:3, less than 1:4, less than 1:5, less than 1:6, less than 1:7, less than 1:8, less than 1:9, less than 1:10, less than 1:20, less than 1:30, less than 1:40, less than 1:50, less than 1:60, less than 1:70, less than 1:80, less than 1:90, or less than 1:100; and/or
s) the Tcon are TCRα⁺, TCRβ⁺, or TCRα⁺TCRβ⁺ and/or the Tcon are CD25⁻, CD127⁺, or CD25⁻CD127⁺ and/or the Tcon are TCRα⁺TCRβ⁺CD45RA⁺CD45RO⁻CD25⁻CD95⁻IL-2Rβ⁻CD127⁺; and/or
t) the population of isolated CD45⁺ cells comprises from less than approximately 5 EU to less than approximately 0.5 EU of endotoxins per mL of formulation; and/or
u) the body weight of the human subject receiving the product is actual body weight or the body weight of the human subject receiving the product is ideal body weight.

4. The cellular therapy product of claim 1, wherein the product further comprises a pharmaceutical composition comprising one or more doses of a graft vs host disease (GVHD) prophylactic agent,
optionally wherein the GVHD prophylactic agent is tacrolimus,
optionally wherein one or more doses of tacrolimus are provided to maintain a trough blood level of approximately 5 ng/mL to approximately 10 ng/mL in the human subject receiving the product,
optionally wherein the pharmaceutical composition comprises tacrolimus at a dose that ranges from approximately 0.01 mg per kilogram of body weight of the human subject receiving the product to approximately 0.50 mg per kilogram of body weight of the human subject receiving the product twice per day,
optionally wherein the tacrolimus is dosed in an amount to maintain or that maintains a target blood level of approximately 1 ng/mL to approximately 10 ng/mL in the human subject receiving the product for approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the population of isolated CD45⁺ cells.

5. The cellular therapy product of claim 1, wherein:
a) the HSPC, the Tmem, the Treg, or any combination thereof are obtained from an allogeneic HLA-matched donor, relative to the human subject receiving the product and/or the population of isolated CD45⁺ cells is from an allogeneic HLA-matched donor, relative to the human subject receiving the product; or
b) wherein the HSPC, the Tmem, the Treg, or any combination thereof are obtained from an allogeneic donor having at least one HLA mismatch, relative to the human subject receiving the product and/or the population of isolated CD45⁺ cells is from an allogeneic donor having at least one HLA mismatch, relative to the human subject receiving the product, optionally wherein the at least one HLA mismatch is at an allele selected from the group consisting of: HLA-A, HLA-B, HLA-C, HLA-DRB1, and any combination thereof, optionally wherein the cells having at least one HLA mismatch are from a donor that is 6/8 HLA-mismatched, relative to the human subject receiving the product, or is 7/8 HLA-mismatched, relative to the human subject receiving the product, optionally wherein the cells having at least one HLA mismatch are from a donor that is 7/8 HLA-mismatched, relative to the human subject receiving the product, optionally wherein the donor that is 7/8 HLA-mismatched, relative to the human subject receiving the product, has a mismatch in HLA-A, optionally wherein the donor that is 7/8 HLA-mismatched, relative to the human subject receiving the product, has a mismatch in HLA-B, optionally wherein the donor that is 7/8 HLA-mismatched, relative to the human subject receiving the product, has a mismatch in HLA-C, optionally wherein the donor that is 7/8 HLA-mismatched, relative to the human subject receiving the product, has a mismatch in HLA-DRB1, optionally wherein the donor that has the at least one HLA mismatch, relative to the human subject receiving the product, has a mismatched HLA allele as a result of the donor being homozygous for the HLA allele while the human subject receiving the product is heterogeneous for the HLA allele, optionally wherein the donor that has at least one HLA mismatch, relative to the human subject receiving the product, has a mismatched HLA allele as a result of the donor being heterozygous for the HLA allele while the human subject receiving the product is homozygous for the HLA allele, optionally wherein the donor that has at least one HLA mismatch, relative to the human subject receiving the product, has a mismatched HLA allele as a result of both the donor and the human subject receiving the product being heterozygous for the HLA allele; or
c) the HSPC, the Tmem, the Treg, or any combination thereof are obtained from an allogeneic haploidentical donor, relative to the human subject receiving the product or the population of isolated CD45⁺ cells is from an allogeneic haploidentical donor, relative to the human subject receiving the product,
optionally wherein the allogeneic donor is related to the human subject receiving the product or the allogeneic donor is unrelated to the human subject receiving the product.

6. The cellular therapy product of claim 1, for use in:
a) a method of treating a human subject having or suspected of having a hematologic malignancy,
optionally wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN), optionally wherein the acute leukemia is in complete remission, optionally wherein the acute leukemia is active acute leukemia; and/or
b) a method of treating a human subject having or suspected of having an autoimmune disorder, optionally wherein the autoimmune disorder is multiple sclerosis, optionally wherein the multiple sclerosis is primary progressive multiple sclerosis (PPMS).

7. A method of treating a human subject:
a) having or suspected of having acute leukemia in complete remission, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon),
optionally wherein the human subject has approximately 5% or less blast burden in their bone marrow, optionally wherein there is an absence of circulating blasts and/or blasts with Auer rods in the human subject, optionally wherein there is an absence of extramedullary disease in the human subject, optionally wherein the human subject has an absolute neutrophil count (ANC) that is approximately 1.0×10⁹/L or higher, optionally wherein the human subject has a platelet count that is approximately 1.0×10¹¹/L or higher, optionally wherein the human subject is red blood cell-transfusion-independent, optionally wherein the acute leukemia is in complete remission with incomplete hematologic recovery, optionally wherein the human subject has residual neutropenia, optionally wherein the human subject has an absolute neutrophil count (ANC) that is approximately 1.0×10⁹/L or lower, optionally wherein the human subject has residual thrombocytopenia, optionally wherein the human subject has a platelet count that is approximately 1.0×10¹¹/L or lower; or
b) having or suspected of having active acute leukemia, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tₘₑₘ), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), optionally wherein the human subject has approximately 10% or less leukemic blast infiltration of bone marrow, optionally wherein the acute leukemia is acute myeloid leukemia (AML), acute lymphoid leukemia (ALL), and/or mixed phenotype acute leukemia (MPAL); or
c) having or suspected of having chronic myelogenous leukemia (CML), the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tₘₑₘ), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), optionally wherein the human subject is in myeloid blast crises and/or lymphoid blast crisis, optionally wherein the blast crisis is in complete remission, optionally wherein the blast crisis is in complete remission with incomplete hematologic recovery, optionally wherein the CML is in accelerated phase or the CML is in chronic phase, optionally wherein the chronic phase CML is resistant to or intolerant of one or more first-generation or second-generation tyrosine kinase inhibitors (TKIs); or
d) having or suspected of having high-risk or very high-risk myelodysplastic syndrome (MDS), the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), optionally the human subject has approximately 10% or less blast burden in their bone marrow; or
e) having or suspected of having myelofibrosis, the method comprising administering to the human subject a cellular therapy product comprising a population of isolated CD45⁺ cells, wherein the population of isolated CD45⁺ cells comprises an enriched population of CD34⁺ hematopoietic stem and progenitor cells (HSPC), an enriched population of CD45RA⁻ memory T cells (Tmem), and an enriched population of fresh CD4⁺CD25⁺CD127^{dim} regulatory T cells (Treg), and wherein the population of isolated CD45⁺ cells has been depleted of naïve conventional CD3⁺CD25⁻CD45RA⁺ T cells (Tcon) to comprise less than approximately 5.0×10⁵ naïve conventional CD3⁺CD25⁻CD45RA⁺ T cells (Tcon) per kilogram of body weight of the human subject receiving the product, less than approximately 6.0×10⁷ total naïve conventional CD3⁺CD25⁻CD45RA⁺ T cells (Tcon), or less than approximately 1.0×10⁶ total naïve conventional CD3⁺CD25⁻CD45RA⁺ T cells (Tcon), optionally wherein the myelofibrosis is eligible for standard myeloablative allogeneic hematopoietic stem cell transplant (alloHSCT), optionally wherein the myelofibrosis is intermediate-2-risk myelofibrosis or high-risk myelofibrosis, optionally wherein the myelofibrosis is intermediate-1-risk myelofibrosis associated with high symptom burden, low platelet counts, and/or complex cytogenetics.

8. The method of claim 7, wherein:
a) the method further comprises administering one or more doses of a graft vs host disease (GVHD) prophylactic agent, optionally wherein the GVHS prophylactic agent is tacrolimus, optionally wherein the tacrolimus is provided in an amount sufficient to maintain a trough blood level of approximately 5 ng/mL to approximately 10 ng/mL in the human subject, optionally wherein the tacrolimus is provided at a dose that ranges from approximately 0.01 mg per kilogram of body weight of the human subject to 0.50 mg per kilogram of body weight of the human subject twice per day, optionally wherein the tacrolimus is dosed in an amount to maintain or that maintains a target blood level of approximately 1 ng/mL to approximately 10 ng/mL in the human subject for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the third population of CD45⁺ cells, optionally wherein the tacrolimus is initially administered from approximately 12 hours to approximately 24 hours after administration of the cellular therapy product, optionally wherein administration of the tacrolimus is tapered starting at approximately 90 days after initial administration of the tacrolimus; and/or
b) the administering comprises infusing into the human subject the population of isolated CD45⁺ cells; and/or
c) the population of isolated CD45⁺ cells comprises from approximately 1.0×10⁵ to approximately 1.0×10⁸ HSPC per kilogram of body weight of the human subject, or from approximately 5.0×10⁵ to approximately 1.5×10¹⁰ HSPC; and/or
d) the population of isolated CD45⁺ cells comprises approximately 1.0×10⁵ or more HSPC per kilogram of body weight of the human subject, approximately 2.0×10⁵ or more HSPC per kilogram of body weight of the human subject, approximately 3.0×105 or more HSPC per kilogram of body weight of the human subject, approximately 4.0×10⁵ or more HSPC per kilogram of body weight of the human subject, approximately 5.0×10⁵ or more HSPC per kilogram of body weight of the human subject, approximately 6.0×10⁵ or more HSPC per kilogram of body weight of the human subject, approximately 7.0×10⁵ or more HSPC per kilogram of body weight of the human subject, approximately 8.0×10⁵ or more HSPC per kilogram of body weight of the human subject, approximately 9.0×10⁵ or more HSPC per kilogram of body weight of the human subject, approximately 1.0×10⁶ or more HSPC per kilogram of body weight of the human subject, approximately 1.5×10⁶ or more HSPC per kilogram of body weight of the human subject, approximately 2.0×10⁶ or more HSPC per kilogram of body weight of the human subject, approximately 2.5×10⁶ or more HSPC per kilogram of body weight of the human subject, approximately 3.0×10⁶ or more HSPC per kilogram of body weight of the human subject, approximately 3.5×10⁶ or more HSPC per kilogram of body weight of the human subject, approximately 4.0×10⁶ or more HSPC per kilogram of body weight of the human subject, approximately 4.5×10⁶ or more HSPC per kilogram of body weight of the human subject, approximately 5.0×10⁶ or more HSPC per kilogram of body weight of the human subject, approximately 5.5×10⁶ or more HSPC per kilogram of body weight of the human subject, approximately 6.0×10⁶ or more HSPC per kilogram of body weight of the human subject, approximately 6.5×10⁶ or more HSPC per kilogram of body weight of the human subject, approximately 7.0×10⁶ or more HSPC per kilogram of body weight of the human subject, approximately 7.5×10⁶ or more HSPC per kilogram of body weight of the human subject, approximately 8.0×10⁶ or more HSPC per kilogram of body weight of the human subject, approximately 8.5×10⁶ or more HSPC per kilogram of body weight of the human subject, approximately 9.0×10⁶ or more HSPC per kilogram of body weight of the human subject, approximately 9.5×10⁶ or more HSPC per kilogram of body weight of the human subject, approximately 1.0×10⁷ or more HSPC per kilogram of body weight of the human subject, approximately 1.5×10⁷ or more HSPC per kilogram of body weight of the human subject, approximately 2.0×10⁷ or more HSPC per kilogram of body weight of the human subject, approximately 2.5×10⁷ or more HSPC per kilogram of body weight of the human subject, approximately 3.0×10⁷ or more HSPC per kilogram of body weight of the human subject, approximately 3.5×10⁷ or more HSPC per kilogram of body weight of the human subject, approximately 4.0×10⁷ or more HSPC per kilogram of body weight of the human subject, approximately 4.5×10⁷ or more HSPC per kilogram of body weight of the human subject, approximately 5.0×10⁷ or more HSPC per kilogram of body weight of the human subject, approximately 5.5×10⁷ or more HSPC per kilogram of body weight of the human subject, approximately 6.0×10⁷ or more HSPC per kilogram of body weight of the human subject, approximately 6.5×10⁷ or more HSPC per kilogram of body weight of the human subject, approximately 7.0×10⁷ or more HSPC per kilogram of body weight of the human subject, approximately 7.5×10⁷ or more HSPC per kilogram of body weight of the human subject, approximately 8.0×10⁷ or more HSPC per kilogram of body weight of the human subject, approximately 8.5×10⁷ or more HSPC per kilogram of body weight of the human subject, approximately 9.0×10⁷ or more HSPC per kilogram of body weight of the human subject, approximately 9.5×10⁷ or more HSPC per kilogram of body weight of the human subject, or approximately 1.0×10⁸ or more HSPC per kilogram of body weight of the human subject; and/or
e) the population of isolated CD45⁺ cells comprises approximately 5.0×10⁵ or more HSPC, approximately 6.0×10⁵ or more HSPC, approximately 7.0×10⁵ or more HSPC, approximately 8.0×10⁵ or more HSPC, approximately 9.0×10⁵ or more HSPC, approximately 1.0×10⁶ or more HSPC, approximately 1.5×10⁶ or more HSPC, approximately 2.0×10⁶ or more HSPC, approximately 2.5×10⁶ or more HSPC, approximately 3.0×10⁶ or more HSPC, approximately 3.5×10⁶ or more HSPC, approximately 4.0×10⁶ or more HSPC, approximately 4.5×10⁶ or more HSPC, approximately 5.0×10⁶ or more HSPC, approximately 5.5×10⁶ or more HSPC, approximately 6.0×10⁶ or more HSPC, approximately 6.5×10⁶ or more HSPC, approximately 7.0×10⁶ or more HSPC, approximately 7.5×10⁶ or more HSPC, approximately 8.0×10⁶ or more HSPC, approximately 8.5×10⁶ or more HSPC, approximately 9.0×10⁶ or more HSPC, approximately 9.5×10⁶ or more HSPC, approximately 1.0×10⁷ or more HSPC, approximately 1.5×10⁷ or more HSPC, approximately 2.0×10⁷ or more HSPC, approximately 2.5×10⁷ or more HSPC, approximately 3.0×10⁷ or more HSPC, approximately 3.5×10⁷ or more HSPC, approximately 4.0×10⁷ or more HSPC, approximately 4.5×10⁷ or more HSPC, approximately 5.0×10⁷ or more HSPC, approximately 5.5×10⁷ or more HSPC, approximately 6.0×10⁷ or more HSPC, approximately 6.5×10⁷ or more HSPC, approximately 7.0×10⁷ or more HSPC, approximately 7.5×10⁷ or more HSPC, approximately 8.0×10⁷ or more HSPC, approximately 8.5×10⁷ or more HSPC, approximately 9.0×10⁷ or more HSPC, approximately 9.5×10⁷ or more HSPC, approximately 1.0×10⁸ or more HSPC, approximately 1.5×10⁸ or more HSPC, approximately 2.0×10⁸ or more HSPC, approximately 2.5×10⁸ or more HSPC, approximately 3.0×10⁸ or more HSPC, approximately 3.5×10⁸ or more HSPC, approximately 4.0×10⁸ or more HSPC, approximately 4.5×10⁸ or more HSPC, approximately 5.0×10⁸ or more HSPC, approximately 5.5×10⁸ or more HSPC, approximately 6.0×10⁸ or more HSPC, approximately 6.5×10⁸ or more HSPC, approximately 7.0×10⁸ or more HSPC, approximately 7.5×10⁸ or more HSPC, approximately 8.0×10⁸ or more HSPC, approximately 8.5×10⁸ or more HSPC, approximately 9.0×10⁸ or more HSPC, approximately 9.5×10⁸ or more HSPC, approximately 1.0×10⁹ or more HSPC, approximately 1.5×10⁹ or more HSPC, approximately 2.0×10⁹ or more HSPC, approximately 2.5×10⁹ or more HSPC, approximately 3.0×10⁹ or more HSPC, approximately 3.5×10⁹ or more HSPC, approximately 4.0×10⁹ or more HSPC, approximately 4.5×10⁹ or more HSPC, approximately 5.0×10⁹ or more HSPC, approximately 5.5×10⁹ or more HSPC, approximately 6.0×10⁹ or more HSPC, approximately 6.5×10⁹ or more HSPC, approximately 7.0×10⁹ or more HSPC, approximately 7.5×10⁹ or more HSPC, approximately 8.0×10⁹ or more HSPC, approximately 8.5×10⁹ or more HSPC, approximately 9.0×10⁹ or more HSPC, approximately 9.5×10⁹ or more HSPC, approximately 1.0×10¹⁰ or more HSPC, or approximately 1.5×10¹⁰ or more HSPC; and/or
f) wherein the population of isolated CD45⁺ cells comprises from approximately 1.0×10⁵ to approximately 1.0×10⁸ Tmem per kilogram of body weight of the human subject receiving the product from approximately 5.0×10⁵ to approximately 1.5×10¹⁰ Tmem; and/or
g) the population of isolated CD45⁺ cells comprises approximately 1.0×10⁵ or more Tmem per kilogram of body weight of the human subject receiving the product, approximately 2.0×10⁵ or more Tmem per kilogram of body weight of the human subject receiving the product, approximately 3.0×10⁵ or more Tmem per kilogram of body weight of the human subject receiving the product, approximately 4.0×10⁵ or more Tmem per kilogram of body weight of the human subject receiving the product, approximately 5.0×10⁵ or more Tmem per kilogram of body weight of the human subject receiving the product, approximately 6.0×10⁵ or more Tmem per kilogram of body weight of the human subject receiving the product, approximately 7.0×10⁵ or more Tmem per kilogram of body weight of the human subject receiving the product, approximately 8.0×10⁵ or more Tmem per kilogram of body weight of the human subject receiving the product, approximately 9.0×10⁵ or more Tmem per kilogram of body weight of the human subject receiving the product, approximately 1.0×10⁶ or more Tmem per kilogram of body weight of the human subject receiving the product, approximately 1.5×10⁶ or more Tmem per kilogram of body weight of the human subject receiving the product, approximately 2.0×10⁶ or more Tmem per kilogram of body weight of the human subject receiving the product, approximately 2.5×10⁶ or more Tmem per kilogram of body weight of the human subject receiving the product, approximately 3.0×10⁶ or more Tmem per kilogram of body weight of the human subject receiving the product, approximately 3.5×10⁶ or more Tmem per kilogram of body weight of the human subject receiving the product, approximately 4.0×10⁶ or more Tmem per kilogram of body weight of the human subject receiving the product, approximately 4.5×10⁶ or more Tmem per kilogram of body weight of the human subject receiving the product, approximately 5.0×10⁶ or more Tmem per kilogram of body weight of the human subject receiving the product, approximately 5.5×10⁶ or more Tmem per kilogram of body weight of the human subject receiving the product, approximately 6.0×10⁶ or more Tmem per kilogram of body weight of the human subject receiving the product, approximately 6.5×10⁶ or more Tmem per kilogram of body weight of the human subject receiving the product, approximately 7.0×10⁶ or more Tmem per kilogram of body weight of the human subject receiving the product, approximately 7.5×10⁶ or more Tmem per kilogram of body weight of the human subject receiving the product, approximately 8.0×10⁶ or more Tmem per kilogram of body weight of the human subject receiving the product, approximately 8.5×10⁶ or more Tmem per kilogram of body weight of the human subject receiving the product, approximately 9.0×10⁶ or more Tmem per kilogram of body weight of the human subject receiving the product, approximately 9.5×10⁶ or more Tmem per kilogram of body weight of the human subject receiving the product, approximately 1.0×10⁷ or more Tmem per kilogram of body weight of the human subject receiving the product, approximately 1.5×10⁷ or more Tmem per kilogram of body weight of the human subject receiving the product, approximately 2.0×10⁷ or more Tmem per kilogram of body weight of the human subject receiving the product, approximately 2.5×10⁷ or more Tmem per kilogram of body weight of the human subject receiving the product, approximately 3.0×10⁷ or more Tmem per kilogram of body weight of the human subject receiving the product, approximately 3.5×10⁷ or more Tmem per kilogram of body weight of the human subject receiving the product, approximately 4.0×10⁷ or more Tmem per kilogram of body weight of the human subject receiving the product, approximately 4.5×10⁷ or more Tmem per kilogram of body weight of the human subject receiving the product, approximately 5.0×10⁷ or more Tmem per kilogram of body weight of the human subject receiving the product, approximately 5.5×10⁷ or more Tmem per kilogram of body weight of the human subject receiving the product, approximately 6.0×10⁷ or more Tmem per kilogram of body weight of the human subject receiving the product, approximately 6.5×10⁷ or more Tmem per kilogram of body weight of the human subject receiving the product, approximately 7.0×10⁷ or more Tmem per kilogram of body weight of the human subject receiving the product, approximately 7.5×10⁷ or more Tmem per kilogram of body weight of the human subject receiving the product, approximately 8.0×10⁷ or more Tmem per kilogram of body weight of the human subject receiving the product, approximately 8.5×10⁷ or more Tmem per kilogram of body weight of the human subject receiving the product, approximately 9.0×10⁷ or more Tmem per kilogram of body weight of the human subject receiving the product, approximately 9.5×10⁷ or more Tmem per kilogram of body weight of the human subject receiving the product, or approximately 1.0×10⁸ or more Tmem per kilogram of body weight of the human subject receiving the product; and/or
h) the population of isolated CD45⁺ cells comprises approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.5×10⁶ or more Tmem, approximately 2.0×10⁶ or more Tmem, approximately 2.5×10⁶ or more Tmem, approximately 3.0×10⁶ or more Tmem, approximately 3.5×10⁶ or more Tmem, approximately 4.0×10⁶ or more Tmem, approximately 4.5×10⁶ or more Tmem, approximately 5.0×10⁶ or more Tmem, approximately 5.5×10⁶ or more Tmem, approximately 6.0×10⁶ or more Tmem, approximately 6.5×10⁶ or more Tmem, approximately 7.0×10⁶ or more Tmem, approximately 7.5×10⁶ or more Tmem, approximately 8.0×10⁶ or more Tmem, approximately 8.5×10⁶ or more Tmem, approximately 9.0×10⁶ or more Tmem, approximately 9.5×10⁶ or more Tmem, approximately 1.0×10⁷ or more Tmem, approximately 1.5×10⁷ or more Tmem, approximately 2.0×10⁷ or more Tmem, approximately 2.5×10⁷ or more Tmem, approximately 3.0×10⁷ or more Tmem, approximately 3.5×10⁷ or more Tmem, approximately 4.0×10⁷ or more Tmem, approximately 4.5×10⁷ or more Tmem, approximately 5.0×10⁷ or more Tmem, approximately 5.5×10⁷ or more Tmem, approximately 6.0×10⁷ or more Tmem, approximately 6.5×10⁷ or more Tmem, approximately 7.0×10⁷ or more Tmem, approximately 7.5×10⁷ or more Tmem, approximately 8.0×10⁷ or more Tmem, approximately 8.5×10⁷ or more Tmem, approximately 9.0×10⁷ or more Tmem, approximately 9.5×10⁷ or more Tmem, approximately 1.0×10⁸ or more Tmem, approximately 1.5×10⁸ or more Tmem, approximately 2.0×10⁸ or more Tmem, approximately 2.5×10⁸ or more Tmem, approximately 3.0×10⁸ or more Tmem, approximately 3.5×10⁸ or more Tmem, approximately 4.0×10⁸ or more Tmem, approximately 4.5×10⁸ or more Tmem, approximately 5.0×10⁸ or more Tmem, approximately 5.5×10⁸ or more Tmem, approximately 6.0×10⁸ or more Tmem, approximately 6.5×10⁸ or more Tmem, approximately 7.0×10⁸ or more Tmem, approximately 7.5×10⁸ or more Tmem, approximately 8.0×10⁸ or more Tmem, approximately 8.5×10⁸ or more Tmem, approximately 9.0×10⁸ or more Tmem, approximately 9.5×10⁸ or more Tmem, approximately 1.0×10⁹ or more Tmem, approximately 1.5×10⁹ or more Tmem, approximately 2.0×10⁹ or more Tmem, approximately 2.5×10⁹ or more Tmem, approximately 3.0×10⁹ or more Tmem, approximately 3.5×10⁹ or more Tmem, approximately 4.0×10⁹ or more Tmem, approximately 4.5×10⁹ or more Tmem, approximately 5.0×10⁹ or more Tmem, approximately 5.5×10⁹ or more Tmem, approximately 6.0×10⁹ or more Tmem, approximately 6.5×10⁹ or more Tmem, approximately 7.0×10⁹ or more Tmem, approximately 7.5×10⁹ or more Tmem, approximately 8.0×10⁹ or more Tmem, approximately 8.5×10⁹ or more Tmem, approximately 9.0×10⁹ or more Tmem, approximately 9.5×10⁹ or more Tmem, approximately 1.0×10¹⁰ or more Tmem, or approximately 1.5×10¹⁰ or more Tmem; and/or
i) the population of isolated CD45⁺ cells comprises from approximately 1.0×10⁵ to approximately 2.0×10⁷ fresh Treg per kilogram of body weight of the human subject or from approximately 5.0×10⁵ to approximately 3.0×10⁹ fresh Treg; and/or
j) the population of isolated CD45⁺ cells comprises approximately 1.0×10⁵ or more fresh Treg per kilogram of body weight of the human subject, approximately 2.0×10⁵ or more fresh Treg per kilogram of body weight of the human subject, approximately 3.0×10⁵ or more fresh Treg per kilogram of body weight of the human subject, approximately 4.0×10⁵ or more fresh Treg per kilogram of body weight of the human subject, approximately 5.0×10⁵ or more fresh Treg per kilogram of body weight of the human subject, approximately 6.0×10⁵ or more fresh Treg per kilogram of body weight of the human subject, approximately 7.0×10⁵ or more fresh Treg per kilogram of body weight of the human subject, approximately 8.0×10⁵ or more fresh Treg per kilogram of body weight of the human subject, approximately 9.0×10⁵ or more fresh Treg per kilogram of body weight of the human subject, approximately 1.0×10⁶ or more fresh Treg per kilogram of body weight of the human subject, approximately 1.5×10⁶ or more fresh Treg per kilogram of body weight of the human subject, approximately 2.0×10⁶ or more fresh Treg per kilogram of body weight of the human subject, approximately 2.5×10⁶ or more fresh Treg per kilogram of body weight of the human subject, approximately 3.0×10⁶ or more fresh Treg per kilogram of body weight of the human subject, approximately 3.5×10⁶ or more fresh Treg per kilogram of body weight of the human subject, approximately 4.0×10⁶ or more fresh Treg per kilogram of body weight of the human subject, approximately 4.5×10⁶ or more fresh Treg per kilogram of body weight of the human subject, approximately 5.0×10⁶ or more fresh Treg per kilogram of body weight of the human subject, approximately 5.5×10⁶ or more fresh Treg per kilogram of body weight of the human subject, approximately 6.0×10⁶ or more fresh Treg per kilogram of body weight of the human subject, approximately 6.5×10⁶ or more fresh Treg per kilogram of body weight of the human subject, approximately 7.0×10⁶ or more fresh Treg per kilogram of body weight of the human subject, approximately 7.5×10⁶ or more fresh Treg per kilogram of body weight of the human subject, approximately 8.0×10⁶ or more fresh Treg per kilogram of body weight of the human subject, approximately 8.5×10⁶ or more fresh Treg per kilogram of body weight of the human subject, approximately 9.0×10⁶ or more fresh Treg per kilogram of body weight of the human subject, approximately 9.5×10⁶ or more fresh Treg per kilogram of body weight of the human subject, approximately 1.0×10⁷ or more fresh Treg per kilogram of body weight of the human subject, approximately 1.5×10⁷ or more fresh Treg per kilogram of body weight of the human subject, or approximately 2.0×10⁷ or more fresh Treg per kilogram of body weight of the human subject; and/or
k) the population of isolated CD45⁺ cells comprises approximately 5.0×10⁵ or more fresh Treg, approximately 6.0×10⁵ or more fresh Treg, approximately 7.0×10⁵ or more fresh Treg, approximately 8.0×10⁵ or more fresh Treg, approximately 9.0×10⁵ or more fresh Treg, approximately 1.0×10⁶ or more fresh Treg, approximately 1.5×10⁶ or more fresh Treg, approximately 2.0×10⁶ or more fresh Treg, approximately 2.5×10⁶ or more fresh Treg, approximately 3.0×10⁶ or more fresh Treg, approximately 3.5×10⁶ or more fresh Treg, approximately 4.0×10⁶ or more fresh Treg, approximately 4.5×10⁶ or more fresh Treg, approximately 5.0×10⁶ or more fresh Treg, approximately 5.5×10⁶ or more fresh Treg, approximately 6.0×10⁶ or more fresh Treg, approximately 6.5×10⁶ or more fresh Treg, approximately 7.0×10⁶ or more fresh Treg, approximately 7.5×10⁶ or more fresh Treg, approximately 8.0×10⁶ or more fresh Treg, approximately 8.5×10⁶ or more fresh Treg, approximately 9.0×10⁶ or more fresh Treg, approximately 9.5×10⁶ or more fresh Treg, approximately 1.0×10⁷ or more fresh Treg, approximately 1.5×10⁷ or more fresh Treg, approximately 2.0×10⁷ or more fresh Treg, approximately 2.5×10⁷ or more fresh Treg, approximately 3.0×10⁷ or more fresh Treg, approximately 3.5×10⁷ or more fresh Treg, approximately 4.0×10⁷ or more fresh Treg, approximately 4.5×10⁷ or more fresh Treg, approximately 5.0×10⁷ or more fresh Treg, approximately 5.5×10⁷ or more fresh Treg, approximately 6.0×10⁷ or more fresh Treg, approximately 6.5×10⁷ or more fresh Treg, approximately 7.0×10⁷ or more fresh Treg, approximately 7.5×10⁷ or more fresh Treg, approximately 8.0×10⁷ or more fresh Treg, approximately 8.5×10⁷ or more fresh Treg, approximately 9.0×10⁷ or more fresh Treg, approximately 9.5×10⁷ or more fresh Treg, approximately 1.0×10⁸ or more fresh Treg, approximately 1.5×10⁸ or more fresh Treg, approximately 2.0×10⁸ or more fresh Treg, approximately 2.5×10⁸ or more fresh Treg, approximately 3.0×10⁸ or more fresh Treg, approximately 3.5×10⁸ or more fresh Treg, approximately 4.0×10⁸ or more fresh Treg, approximately 4.5×10⁸ or more fresh Treg, approximately 5.0×10⁸ or more fresh Treg, approximately 5.5×10⁸ or more fresh Treg, approximately 6.0×10⁸ or more fresh Treg, approximately 6.5×10⁸ or more fresh Treg, approximately 7.0×10⁸ or more fresh Treg, approximately 7.5×10⁸ or more fresh Treg, approximately 8.0×10⁸ or more fresh Treg, approximately 8.5×10⁸ or more fresh Treg, approximately 9.0×10⁸ or more fresh Treg, approximately 9.5×10⁸ or more fresh Treg, approximately 1.0×10⁹ or more fresh Treg, approximately 1.5×10⁹ or more fresh Treg, approximately 2.0×10⁹ or more fresh Treg, approximately 2.5×10⁹ or more fresh Treg, or approximately 3.0×10⁹ or more fresh Treg; and/or
l) the population of isolated CD45⁺ cells comprises less than approximately 5.0×10⁵ Tcon per kilogram of body weight of the human subject receiving the product, less than approximately 4.5×10⁵ Tcon per kilogram of body weight of the human subject receiving the product, less than approximately 4.0×10⁵ Tcon per kilogram of body weight of the human subject receiving the product, less than approximately 3.5×10⁵ Tcon per kilogram of body weight of the human subject receiving the product, less than approximately 3.0×10⁵ Tcon per kilogram of body weight of the human subject receiving the product, less than approximately 2.5×10⁵ Tcon per kilogram of body weight of the human subject receiving the product, less than approximately 2.0×10⁵ Tcon per kilogram of body weight of the human subject receiving the product, less than approximately 1.5×10⁵ Tcon per kilogram of body weight of the human subject receiving the product, less than approximately 1.0×10⁵ Tcon per kilogram of body weight of the human subject receiving the product, less than approximately 9.5×10⁴ Tcon per kilogram of body weight of the human subject receiving the product, less than approximately 9.0×10⁴ Tcon per kilogram of body weight of the human subject receiving the product, less than approximately 8.5×10⁴ Tcon per kilogram of body weight of the human subject receiving the product, less than approximately 8.0×10⁴ Tcon per kilogram of body weight of the human subject receiving the product, less than approximately 7.5×10⁴ Tcon per kilogram of body weight of the human subject receiving the product, less than approximately 7.0×10⁴ Tcon per kilogram of body weight of the human subject receiving the product, less than approximately 6.5×10⁴ Tcon per kilogram of body weight of the human subject receiving the product, less than approximately 6.0×10⁴ Tcon per kilogram of body weight of the human subject receiving the product, less than approximately 5.5×10⁴ Tcon per kilogram of body weight of the human subject receiving the product, less than approximately 5.0×10⁴ Tcon per kilogram of body weight of the human subject receiving the product, less than approximately 4.5×10⁴ Tcon per kilogram of body weight of the human subject receiving the product, less than approximately 4.0×10⁴ Tcon per kilogram of body weight of the human subject receiving the product, less than approximately 3.5×10⁴ Tcon per kilogram of body weight of the human subject receiving the product, less than approximately 3.0×10⁴ Tcon per kilogram of body weight of the human subject receiving the product, less than approximately 2.5×10⁴ Tcon per kilogram of body weight of the human subject receiving the product, less than approximately 2.0×10⁴ Tcon per kilogram of body weight of the human subject receiving the product, less than approximately 1.5×10⁴ Tcon per kilogram of body weight of the human subject receiving the product, less than approximately 1.0×10⁴ Tcon per kilogram of body weight of the human subject receiving the product, less than approximately 9.5×10³ Tcon per kilogram of body weight of the human subject receiving the product, less than approximately 9.0×10³ Tcon per kilogram of body weight of the human subject receiving the product, less than approximately 8.5×10³ Tcon per kilogram of body weight of the human subject receiving the product, less than approximately 8.0×10³ Tcon per kilogram of body weight of the human subject receiving the product, less than approximately 7.5×10³ Tcon per kilogram of body weight of the human subject receiving the product, less than approximately 7.0×10³ Tcon per kilogram of body weight of the human subject receiving the product, less than approximately 6.5×10³ Tcon per kilogram of body weight of the human subject receiving the product, less than approximately 6.0×10³ Tcon per kilogram of body weight of the human subject receiving the product, less than approximately 5.5×10³ Tcon per kilogram of body weight of the human subject receiving the product, less than approximately 5.0×10³ Tcon per kilogram of body weight of the human subject receiving the product, less than approximately 4.5×10³ Tcon per kilogram of body weight of the human subject receiving the product, less than approximately 4.0×10³ Tcon per kilogram of body weight of the human subject receiving the product, less than approximately 3.5×10³ Tcon per kilogram of body weight of the human subject receiving the product, less than approximately 3.0×10³ Tcon per kilogram of body weight of the human subject receiving the product, less than approximately 2.5×10³ Tcon per kilogram of body weight of the human subject receiving the product, less than approximately 2.0×10³ Tcon per kilogram of body weight of the human subject receiving the product, less than approximately 1.5×10³ Tcon per kilogram of body weight of the human subject receiving the product, or less than approximately 1.0×10³ Tcon per kilogram of body weight of the human subject receiving the product; and/or
m) the population of isolated CD45⁺ cells comprises less than approximately 6.0×10⁷ Tcon, less than approximately 5.5×10⁷ Tcon, less than approximately 5.0×10⁷ Tcon, less than approximately 4.5×10⁷ Tcon, less than approximately 4.0×10⁷ Tcon, less than approximately 3.5×10⁷ Tcon, less than approximately 3.0×10⁷ Tcon, less than approximately 2.5×10⁷ Tcon, less than approximately 2.0×10⁷ Tcon, less than approximately 1.5×10⁷ Tcon, less than approximately 1.0×10⁷ Tcon, less than approximately 9.5×10⁶ Tcon, less than approximately 9.0×10⁶ Tcon, less than approximately 8.5×10⁶ Tcon, less than approximately 8.0×10⁶ Tcon, less than approximately 7.5×10⁶ Tcon, less than approximately 7.0×10⁶ Tcon, less than approximately 6.5×10⁶ Tcon, less than approximately 6.0×10⁶ Tcon, less than approximately 5.5×10⁶ Tcon, less than approximately 5.0×10⁶ Tcon, less than approximately 4.5×10⁶ Tcon, less than approximately 4.0×10⁶ Tcon, less than approximately 3.5×10⁶ Tcon, less than approximately 3.0×10⁶ Tcon, less than approximately 2.5×10⁶ Tcon, less than approximately 2.0×10⁶ Tcon, less than approximately 1.5×10⁶ Tcon, or less than approximately 1.0×10⁶ Tcon; and/or
n) the population of isolated CD45⁺ cells further comprises an enriched population of TCR Vα24Jα18⁺CD127⁺ invariant Natural Killer T cells (iNKT), optionally wherein the population of isolated CD45⁺ cells comprises from approximately 5.0×10² to approximately 2.0×10⁶ iNKT per kilogram of body weight of the human subject receiving the product or from approximately 2.0×10³ to approximately 4.0×10⁸ iNKT, optionally wherein the population of isolated CD45⁺ cells comprises approximately 5.0×10² or more iNKT per kilogram of body weight of the human subject receiving the product, approximately 6.0×10² or more iNKT per kilogram of body weight of the human subject receiving the product, approximately 7.0×10² or more iNKT per kilogram of body weight of the human subject receiving the product, approximately 8.0×10² or more iNKT per kilogram of body weight of the human subject receiving the product, approximately 9.0×10² or more iNKT per kilogram of body weight of the human subject receiving the product, approximately 1.0×10³ or more iNKT per kilogram of body weight of the human subject receiving the product, approximately 2.0×10³ or more iNKT per kilogram of body weight of the human subject receiving the product, approximately 3.0×10³ or more iNKT per kilogram of body weight of the human subject receiving the product, approximately 4.0×10³ or more iNKT per kilogram of body weight of the human subject receiving the product, approximately 5.0×10³ or more iNKT per kilogram of body weight of the human subject receiving the product, approximately 6.0×10³ or more iNKT per kilogram of body weight of the human subject receiving the product, approximately 7.0×10³ or more iNKT per kilogram of body weight of the human subject receiving the product, approximately 8.0×10³ or more iNKT per kilogram of body weight of the human subject receiving the product, approximately 9.0×10³ or more iNKT per kilogram of body weight of the human subject receiving the product, approximately 1.0×10⁴ or more iNKT per kilogram of body weight of the human subject receiving the product, approximately 2.0×10⁴ or more iNKT per kilogram of body weight of the human subject receiving the product, approximately 3.0×10⁴ or more iNKT per kilogram of body weight of the human subject receiving the product, approximately 4.0×10⁴ or more iNKT per kilogram of body weight of the human subject receiving the product, approximately 5.0×10⁴ or more iNKT per kilogram of body weight of the human subject receiving the product, approximately 6.0×10⁴ or more iNKT per kilogram of body weight of the human subject receiving the product, approximately 7.0×10⁴ or more iNKT per kilogram of body weight of the human subject receiving the product, approximately 8.0×10⁴ or more iNKT per kilogram of body weight of the human subject receiving the product, approximately 9.0×10⁴ or more iNKT per kilogram of body weight of the human subject receiving the product, approximately 1.0×10⁵ or more iNKT per kilogram of body weight of the human subject receiving the product, approximately 2.0×10⁵ or more iNKT per kilogram of body weight of the human subject receiving the product, approximately 3.0×10⁵ or more iNKT per kilogram of body weight of the human subject receiving the product, approximately 4.0×10⁵ or more iNKT per kilogram of body weight of the human subject receiving the product, approximately 5.0×10⁵ or more iNKT per kilogram of body weight of the human subject receiving the product, approximately 6.0×10⁵ or more iNKT per kilogram of body weight of the human subject receiving the product, approximately 7.0×10⁵ or more iNKT per kilogram of body weight of the human subject receiving the product, approximately 8.0×10⁵ or more iNKT per kilogram of body weight of the human subject receiving the product, approximately 9.0×10⁵ or more iNKT per kilogram of body weight of the human subject receiving the product, approximately 1.0×10⁶ or more iNKT per kilogram of body weight of the human subject receiving the product, approximately 1.5×10⁶ or more iNKT per kilogram of body weight of the human subject receiving the product, or approximately 2.0×10⁶ or more iNKT per kilogram of body weight of the human subject receiving the product, optionally wherein the population of isolated CD45⁺ cells comprises approximately 2.0×10³ or more iNKT, approximately 3.0×10³ or more iNKT, approximately 4.0×10³ or more iNKT, approximately 5.0×10³ or more iNKT, approximately 6.0×10³ or more iNKT, approximately 7.0×10³ or more iNKT, approximately 8.0×10³ or more iNKT, approximately 9.0×10³ or more iNKT, approximately 1.0×10⁴ or more iNKT, approximately 2.0×10⁴ or more iNKT, approximately 3.0×10⁴ or more iNKT, approximately 4.0×10⁴ or more iNKT, approximately 5.0×10⁴ or more iNKT, approximately 6.0×10⁴ or more iNKT, approximately 7.0×10⁴ or more iNKT, approximately 8.0×10⁴ or more iNKT, approximately 9.0×10⁴ or more iNKT, approximately 1.0×10⁵ or more iNKT, approximately 2.0×10⁵ or more iNKT, approximately 3.0×10⁵ or more iNKT, approximately 4.0×10⁵ or more iNKT, approximately 5.0×10⁵ or more iNKT, approximately 6.0×10⁵ or more iNKT, approximately 7.0×10⁵ or more iNKT, approximately 8.0×10⁵ or more iNKT, approximately 9.0×10⁵ or more iNKT, approximately 1.0×10⁶ or more iNKT, approximately 1.5×10⁶ or more iNKT, approximately 2.0×10⁶ or more iNKT, approximately 2.5×10⁶ or more iNKT, approximately 3.0×10⁶ or more iNKT, approximately 3.5×10⁶ or more iNKT, approximately 4.0×10⁶ or more iNKT, approximately 4.5×10⁶ or more iNKT, approximately 5.0×10⁶ or more iNKT, approximately 5.5×10⁶ or more iNKT, approximately 6.0×10⁶ or more iNKT, approximately 6.5×10⁶ or more iNKT, approximately 7.0×10⁶ or more iNKT, approximately 7.5×10⁶ or more iNKT, approximately 8.0×10⁶ or more iNKT, approximately 8.5×10⁶ or more iNKT, approximately 9.0×10⁶ or more iNKT, approximately 9.5×10⁶ or more iNKT, approximately 1.0×10⁷ or more iNKT, approximately 1.5×10⁷ or more iNKT, approximately 2.0×10⁷ or more iNKT, approximately 2.5×10⁷ or more iNKT, approximately 3.0×10⁷ or more iNKT, approximately 3.5×10⁷ or more iNKT, approximately 4.0×10⁷ or more iNKT, approximately 4.5×10⁷ or more iNKT, approximately 5.0×10⁷ or more iNKT, approximately 5.5×10⁷ or more iNKT, approximately 6.0×10⁷ or more iNKT, approximately 6.5×10⁷ or more iNKT, approximately 7.0×10⁷ or more iNKT, approximately 7.5×10⁷ or more iNKT, approximately 8.0×10⁷ or more iNKT, approximately 8.5×10⁷ or more iNKT, approximately 9.0×10⁷ or more iNKT, approximately 9.5×10⁷ or more iNKT, approximately 1.0×10⁸ or more iNKT, approximately 1.5×10⁸ or more iNKT, approximately 2.0×10⁸ or more iNKT, approximately 2.5×10⁸ or more iNKT, approximately 3.0×10⁸ or more iNKT, approximately 3.5×10⁸ or more iNKT, or approximately 4.0×10⁸ or more iNKT, optionally wherein the iNKT are CD1d-tet+, 6B11⁺, or CD1d-tet⁺6B11⁺; and/or
o) wherein the population of isolated CD45⁺ cells is from an allogeneic donor having at least one HLA mismatch relative to the human subject; and/or
p) the HSPC are cKIT⁺, CD133⁺, CD90⁺, CD38⁻, CD45RA⁻, Lin⁻, CD19⁻, TCRα⁻, or any combination thereof, and/or
q) the Tmem are CD3⁺CD45RO⁺; and/or
r) the Tmem comprise a population of T central memory cells (T_{CM}), a population of T effector memory cells (T_{EM}), a population of T stem central memory cells (T_{SCM}), or any combination thereof, optionally wherein the T_{CM} are CD4⁺CD45RO⁺ or CD8⁺CD45RO⁺ and/or the T_{CM} are CD45RA⁻, CD62L⁺, CCR7⁺, or any combination thereof, optionally wherein the T_{EM} are CD4⁺, CD45RO⁺, CD45RA⁻, CD62L⁻, CCR7⁻, or any combination thereof, optionally wherein the T_{SCM} are CD4⁺CD45RA⁺ or CD8⁺CD45RA⁺ and/or the T_{SCM} are CD95⁺, CD122⁺, CXCR3⁺, LFA-1⁺, or any combination thereof, and/or
s) the Treg are FOXP3⁺; and/or
t) the Treg comprise a population of naïve Treg cells, a population of memory Treg cells, or a population of naïve Treg cells and memory Treg cells, optionally wherein the naïve Treg cells are CD4⁺CD25⁺CD127^{dim}FoxP3⁺CD45RA⁺CD45RO⁻, optionally wherein the Treg comprise from approximately 2.0×10⁵ to approximately 5.0×10⁸ naïve Treg cells per kilogram of body weight of the human subject receiving the product, or from approximately 8.0×10⁵ to approximately 1.0×10¹¹ naïve Treg cells, optionally wherein the memory Treg cells are CD4⁺CD25⁺CD127^{dim}FoxP3⁺CD45RA⁻CD45RO⁺, optionally wherein the Treg comprise from approximately 5.0×10⁴ to approximately 5.0×10⁸ memory Treg cells per kilogram of body weight of the human subject receiving the product, or from approximately 2.0×10⁵ to approximately 1.0×10¹¹ memory Treg cells; and/or
u) the Tcon are TCRα⁺, TCRβ⁺, or TCRα⁺TCRβ⁺ and/or the Tcon are CD25⁻, CD127⁺, or CD25⁻CD127⁺ and/or the Tcon are TCRα⁺TCRβ⁺CD45RA⁺CD45RO⁻CD25⁻CD95−IL-2Rβ⁻CD127⁺; and/or
v) the population of isolated CD45⁺ cells comprises from less than approximately 5 EU to less than approximately 0.5 EU of endotoxins per mL of formulation; and/or
w) the HSPC, the Tmem, the Treg, or any combination thereof are obtained from an allogeneic HLA-matched donor relative to the human subject receiving the product or the population of isolated CD45⁺ cells is from an allogeneic HLA-matched donor relative to the human subject receiving the product; and/or
x) the HSPC, the Tmem, the Treg, or any combination thereof are obtained from an allogeneic donor having at least one HLA mismatch relative to the human subject receiving the product or the population of isolated CD45⁺ cells is from an allogeneic donor having at least one HLA mismatch relative to the human subject receiving the product, optionally wherein the at least one HLA mismatch is at an allele selected from the group consisting of: HLA-A, HLA-B, HLA-C, HLA-DRB1, and any combination thereof, optionally wherein the cells having at least one HLA mismatch are from a donor that is 6/8 HLA-mismatched relative to the human subject receiving the product or is 7/8 HLA-mismatched relative to the human subject receiving the product, optionally wherein the cells having at least one HLA mismatch are from a donor that is 7/8 HLA-mismatched relative to the human subject receiving the product, optionally wherein the donor that is 7/8 HLA-mismatched relative to the human subject receiving the product has a mismatch in HLA-A, optionally wherein the donor that is 7/8 HLA-mismatched relative to the human subject receiving the product has a mismatch in HLA-B, optionally wherein the donor that is 7/8 HLA-mismatched relative to the human subject receiving the product has a mismatch in HLA-C, optionally wherein the donor that is 7/8 HLA-mismatched relative to the human subject receiving the product has a mismatch in HLA-DRB1, optionally wherein the donor that has the at least one HLA mismatch relative to the human subject receiving the product has a mismatched HLA allele as a result of the donor being homozygous for the HLA allele while the human subject receiving the product is heterogeneous for the HLA allele, optionally wherein the donor that has at least one HLA mismatch relative to the human subject receiving the product has a mismatched HLA allele as a result of the donor being heterozygous for the HLA allele while the human subject receiving the product is homozygous for the HLA allele, optionally wherein the donor that has at least one HLA mismatch relative to the human subject receiving the product has a mismatched HLA allele as a result of both the donor and the human subject receiving the product being heterozygous for the HLA allele; and/or
y) the HSPC, the Tmem, the Treg, or any combination thereof are obtained from an allogeneic haploidentical donor relative to the human subject receiving the product or the population of isolated CD45⁺ cells is from an allogeneic haploidentical donor relative to the human subject receiving the product; and/or
z) the allogeneic donor is related to the human subject receiving the product or the allogeneic donor is unrelated to the human subject receiving the product; and/or
aa) overall survival rate 12 months or more after administration of the cellular therapy product ranges from approximately 75% to approximately 100%; and/or
bb) overall survival of the human subject increases after administration of the cellular therapy product, relative to a corresponding human subject that has been administered a standard myeloablative allogeneic hematopoietic stem cell transplant (alloHSCT); and/or
cc) GVHD and relapse-free survival (GFRS) rate 12 months or more after administration of the cellular therapy product ranges from approximately 65% to approximately 100%; and/or
dd) GVHD and relapse-free survival (GFRS) of the human subject increases after administration of the cellular therapy product, relative to a corresponding human subject that has been administered a standard myeloablative allogeneic hematopoietic stem cell transplant (alloHSCT); and/or
ee) relapse-free survival rate 12 months or more after administration of the cellular therapy product ranges from approximately 70% to approximately 100%; and/or
ff) relapse-free survival of the human subject increases after administration of the cellular therapy product, relative to a corresponding human subject that has been administered a standard myeloablative allogeneic hematopoietic stem cell transplant (alloHSCT); and/or
gg) wherein incidence of acute GVHD 100 or more days after administration of the cellular therapy product ranges from approximately 9% to approximately 31%; and/or
hh) incidence of chronic GVHD 100 or more days after administration of the cellular therapy product ranges from approximately 1% to approximately 10%; and/or
ii) incidence of post-transplant lymphoproliferative disorder (PTLD) 100 or more days after administration of the cellular therapy product ranges from approximately 1% to approximately 10%; and/or
jj) incidence of Grade 2 or higher infections 100 or more days after administration of the cellular therapy product ranges from approximately 23% to approximately 31%.

9. The method of claim 7, wherein the method further comprises administering a conditioning regimen prior to administration of the cellular therapy product, optionally wherein the conditioning regimen is administered from approximately two days to approximately ten days before administration of the multi-component cellular therapy, optionally wherein the conditioning regimen is a total body irradiation-based (TBI-based) regimen or a total lymphatic irradiation-based (TLI-based) regimen, optionally wherein the TBI-based regimen comprises fractionated total body irradiation, optionally wherein the fractionated total body irradiation comprises 2 or more, 3 or more, 4 or more, 5 or more, 6 or more, 7 or more, 8 or more, 9 or more, 10 or more, 11 or more, 12 or more, 13 or more, 14 or more, or 15 or more doses, optionally wherein the fractionated total body irradiation comprises a total dose that ranges from approximately 500 to approximately 1600 cGy, optionally wherein the TBI-based regimen further comprises one or more conditioning reagents, optionally wherein the one or more conditioning reagents are selected from the group consisting of: cyclophosphamide, etoposide, fludarabine, thiotepa, anti-thymocyte globulin (ATG), and any combination thereof, optionally wherein the conditioning regimen is a myeloablative conditioning regimen, optionally wherein the myeloablative conditioning regimen comprises one or more conditioning reagents, optionally wherein the one or more conditioning reagents are selected from the group consisting of: thiotepa, busulfan, melphalan, fludarabine, cyclophosphamide, anti-thymocyte globulin (ATG), and any combination thereof, optionally wherein the myeloablative conditioning regimen comprises three or more conditioning reagents, wherein at least one conditioning reagent comprises thiotepa, optionally wherein the myeloablative conditioning regimen comprises one or more doses of busulfan, fludarabine, and thiotepa, optionally wherein the one or more doses of busulfan, fludarabine, and thiotepa comprise from approximately 5 to approximately 12 mg of thiotepa per kilogram of body weight of the human subject, from approximately 7 to approximately 11 mg of busulfan per kilogram of body weight of the human subject, and from approximately 100 to approximately 200 mg of fludarabine per meter² body surface area respectively.

10. The method of claim 7, wherein the method further comprises collecting one or more mobilized peripheral blood donations from the donor, optionally wherein the peripheral blood donations are mobilized by granulocyte colony-stimulating factor (G-CSF), granulocyte macrophage colony-stimulating factor (GM-CSF), plerixafor, or any combination thereof, optionally wherein at least one of the mobilized peripheral blood donations is processed and sorted using one or more immune-separation particles (ISPs) to enrich HSPC, Tmem, and Treg, optionally wherein the one or more ISPs comprise affinity reagents, optionally wherein the affinity reagents are immuno-magnetic separation particles, optionally wherein the immuno-magnetic separation particles are antibodies each conjugated to an iron-containing particle, optionally wherein the affinity reagents comprise a plurality of CD34-reagents that binds to CD34 on an HSPC, optionally wherein an average number of ISP's per HSPC in the population of isolated CD45⁺ cells is equal to or less than approximately 20,000, optionally wherein an average number of ISP's per HSPC in the population of isolated CD45⁺ cells is from approximately 1000 to approximately 20,000, optionally wherein the affinity reagents comprise a plurality of CD45RA-reagents that binds to one or more CD45RA receptors on a Tmem, optionally wherein the affinity reagents comprise a plurality of CD25-reagents that binds to one or more CD25 receptors on a Treg, optionally wherein an average number of ISPs per Treg in the population of isolated CD45⁺ cells is equal or less than approximately 4000, optionally wherein an average number of ISPs per Treg in the population of isolated CD45⁺ cells is from approximately 1500 to approximately 2500

11. The method of claim 7, wherein:
a) the human subject is approximately 3 months of age or older; and/or
b) the human subject is from between approximately 3 months to approximately 18 years of age; and/or
c) the human subject is approximately 12 years of age or older; and/or
d) the human subject is between approximately 12 years to approximately 65 years of age; and/or
e) the human subject is between approximately 18 years to approximately 65 years of age; and/or
f) the human subject is between approximately 12 years to approximately 75 years of age; and/or
g) the human subject is from approximately 65 years of age to approximately 75 years of age; and/or
h) the human subject is from between approximately 3 months to approximately 75 years of age; and/or
i) the human subject has received from one to five previous lines of therapy.

12. A cellular therapy kit comprising the cellular therapy product of claim 1, optionally wherein the kit further comprises written instructions for using the cellular therapy for treating a hematologic malignancy in a human subject, optionally wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).

13. A unit dose comprising the cellular therapy product of claim 1, optionally wherein the population of isolated CD45⁺ cells further comprises one or more excipients.

14. An article of manufacture comprising the cellular therapy product of claim 1, optionally wherein the article of manufacture further comprises instructions for administering the article of manufacture to a human subject treat a hematologic malignancy, optionally wherein the hematologic malignancy is selected from the group consisting of: leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome (MDS), myeloproliferative syndrome, myelofibrosis, and blastic plasmacytoid dendritic cell neoplasm (BPDCN).

15. A container comprising the cellular therapy product of claim 1.
